(19) United States
(12) Patent Application Publication
(10) Pub. No.: US 2026/0098049 A1
(43) Pub. Date: Apr. 9, 2026

(54) KRAS MODULATING COMPOUNDS

(71) Applicant: Gilead Sciences, Inc., Foster City, CA (US)

(72) Inventors: Eda Y. Canales, San Mateo, CA (US); Christopher T. Clark, Seattle, WA (US); Zhimin Du, Belmont, CA (US); Yassin M.A. Elbatrawi, Burlingame, CA (US); Quinn B. Greven, San Francisco, CA (US); Tezcan Guney, Foster City, CA (US); Darryl Kato, San Francisco, CA (US); Henok H. Kinfe, Foster City, CA (US); Irene N. Kiburu, San Francisco, CA (US); Scott E. Lazerwith, San Francisco, CA (US); Thomas C.J. Mies, Burlingame, CA (US); Christopher J. Swank, San Mateo, CA (US); Rhiannon Thomas-Tran, San Jose, CA (US); Guanghu Tong, Foster City, CA (US); William J. Watkins, Saratoga, CA (US); Wilian Augusto Cortopassi Coelho, San Francisco, CA (US)

(21) Appl. No.: 19/296,626

(22) Filed: Aug. 11, 2025

Related U.S. Application Data
(60) Provisional application No. 63/682,107, filed on Aug. 12, 2024.

(51) Int. Cl. C07D 519/00; A61K 31/553

(52) U.S. Cl. CPC C07D 519/00; A61K 31/553

(57) ABSTRACT

Provided herein are compounds, and pharmaceutically acceptable salts thereof, useful as KRAS inhibitors, methods of making and using the same (singly or in combination with additional agents), and pharmaceutical compositions thereof.

### CROSS-REFERENCE TO RELATED APPLICATIONS

This application claims the benefit under 35 U.S.C. § 119(e) of U.S. Provisional Application No. 63/682,107, filed Aug. 12, 2024, which is incorporated herein by reference in its entirety for all purposes.

### BACKGROUND

The KRAS protein, Kirsten Rat Sarcoma 2 Viral Oncogene Homolog (“KRAS”), is a GTPase. KRAS gene mutations have been observed in a number of conditions including, for instance, pancreatic cancer, endometrial cancer, lung adenocarcinoma, colorectal cancer, rectal carcinoma, gall bladder cancer, thyroid cancer, bile duct cancer, small cell lung cancer, and non-small cell lung cancer (NSCLC). Accordingly, there is a need for compounds, pharmaceutical compositions, and methods for inhibiting KRAS (e.g., wild type, KRAS G12C, KRAS G12D, and/or KRAS G12V) and treating associated cancers.

### SUMMARY

In one embodiment, the present disclosure provides a compound of Formula (I):

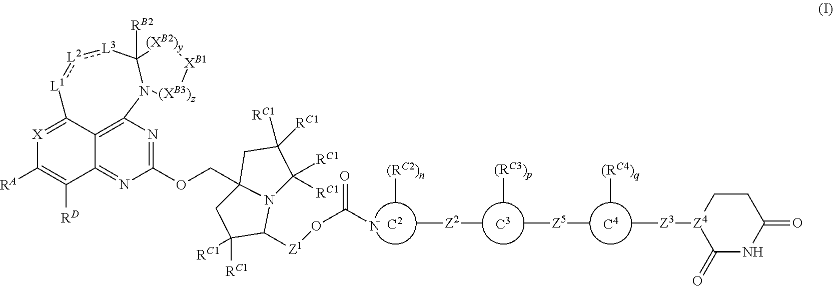

or a pharmaceutically acceptable salt thereof,
wherein
each R^{C1} is independently H, C₁-C₆ alkyl, C₂-C₆ alkoxyalkyl, C₁-C₆ hydroxyalkyl, halo, C₁-C₆ haloalkyl, C₁-C₆ haloalkoxy, —OH, —CN, C₁-C₆ cyanoalkyl, C₃-C₆ cycloalkyl, ═CH₂, ═CHF, or ═CF₂, or
alternatively, two R^{C1} attached to the same carbon form a C₃-C₆-membered cycloalkyl or 4- to 6-membered heterocyclyl, wherein the cycloalkyl and heterocyclyl is substituted by 0, 1, or 2 halo;
Z¹ is —(CR^{2f}R^{2g})ₘ—;
R^{2f} and R^{2g} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2f} and R^{2g} can combine with the carbon to which they are attached to form a C³-C⁶ cycloalkyl;
m is 1, 2, or 3;
ring C2 is 4- to 16-membered heterocyclyl having at least one ring nitrogen;
each R^{C2} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, —CN, C₁-C₆ cyanoalkyl, or C₃-C₆-cycloalkyl;
n is 0, 1, 2, 3, or 4;
Z² is C₁-C₃ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z2b}—, —C(O)—, —S(O)—, S(O)₂—, S(O)(═NR^{Z2})—, oxetanyl, or a bond; wherein the alkyl and cycloalkyl is substituted with 0, 1, 2, 3, or 4 Z^{2a}; wherein the alkenyl is substituted with 0, 1, or 2 Z^{2c};
each Z^{2a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
each Z^{2c} is independently C₁-C₆ alkyl, halo, C₁-C₆ haloalkyl, or C₃-C₆ cycloalkyl;
R^{Z2} is H, C₁-C₆ alkyl, C₁-C₆ haloalkyl, or C₃-C₆ cycloalkyl;
R^{Z2b} is H, C₁-C₆ alkyl, C₁-C₆ haloalkyl, or C₃-C₆ cycloalkyl;
ring C3 is C₃-C₈ cycloalkyl, 4- to 16-membered heterocyclyl, C₆-C₁₀ aryl, 5- to 10-membered heteroaryl, or absent;
each R^{C3} is independently C₁-C₆ alkyl, C₂-C₆ alkenyl, C₂-C₆ alkynyl, halo, C₁-C₆ haloalkyl, C₁-C₆ haloalkoxy, C₁-C₆ thioalkyl, C₁-C₆ thiohaloalkyl, —OH, oxo, —CN, C₁-C₆ alkoxy, C₂-C₆ alkoxyalkyl, C₁-C₆ hydroxyalkyl, C₁-C₆ cyanoalkyl, or C₃-C₆-cycloalkyl;
p is 0, 1, 2, 3, or 4;
Z⁵ is C₁-C₃ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z5b}—, —C(O)—, —S(O)—, S(O)₂—, S(O)(═NR^{Z5})—, or a bond; wherein the alkyl and cycloalkyl is substituted with 0, 1, 2, 3, or 4 Z^{5a};
each Z^{5a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z5} is H, C₁-C₆ alkyl, C₁-C₆ haloalkyl or C₃-C₆ cycloalkyl;
R^{Z5b} is H, C₁-C₆ alkyl, C₁-C₆ haloalkyl, or C₃-C₆ cycloalkyl;
ring C4 is 5 to 14-membered heterocyclyl, C₆-C₁₀ aryl, or 5- to 10-membered heteroaryl;
Z³ is —NH—, —C(O)NH—, or a bond, and Z⁴ is —CH—;
alternatively, Z³ is a bond and Z⁴ is N;
each R^{C4} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, —CN, C₁-C₃ cyanoalkyl, C₃-C₆ cycloalkyl, or 4- to 10-membered heterocyclyl;
q is 0, 1, 2, 3, or 4;
X is N, CH, or CR^{x};
R^{x} is halo, C₁-C₃ haloalkyl, or C₁-C₃ cyanoalkyl;
L¹ is O, CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6-membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2c})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
such that when L² is ═C(R^{2e})— then L¹ is —C(R^{1e})═ or L³ is ═C(R^{3e})—, and when L³ is ═C(R^{3e})— then L² is ═C(R^{2e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆haloalkyl, —CN, —C(O)R^{A2a}, —C(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, —S(O)₂OR^{A2a}, —N(R^{A2a})(R^{A2b}), —(C₀-C₃ alkyl)-SF₅, C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, —SR^{A3a}—N(R^{A3a})(R^{A3b}), C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ thiohaloalkyl, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} attached to two different atoms can combine to form a C₃-C₁₀ cycloalkyl or 4- to 10-membered heterocyclyl, wherein the cycloalkyl and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
R^{D} is halo;
wherein, unless otherwise indicated, each heterocyclyl has 1, 2, 3, or 4 heteroatoms selected from N, O, S, and Si; and
each heteroaryl has 1, 2, 3, or 4 heteroatoms selected from N, O, and S.

In another embodiment, the present disclosure provides a pharmaceutical composition comprising a compound of the present disclosure, and a pharmaceutically acceptable excipient.

In another embodiment, the present disclosure provides a method of inhibiting KRAS wild type, G12C, G12D, or G12V protein in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of degrading KRAS wild type, G12C, G12D, or G12V protein in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of treating cancer in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

Also disclosed herein are compounds and pharmaceutically acceptable salts thereof of sub-formulas of Formula (I).

### DETAILED DESCRIPTION

### I. General

The disclosure relates generally to methods and compounds, and pharmaceutically acceptable salts thereof, for inhibiting KRAS wild type, KRAS^{G12D}, KRAS^{G12C} and/or KRAS^{G12V}. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

A squiggly line on a chemical group as shown below, for example,

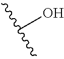 [SMILES: *O]

indicates a point of attachment, i.e., it shows the broken bond by which the group is connected to another described group.

As used herein, “a compound of the disclosure” can mean a compound of any of the Formula (I), (Ia), and/or (Ib), or a pharmaceutically acceptable salt thereof. Similarly, the phrase “a compound of Formula (number)” means a compound of that formula and pharmaceutically acceptable salts thereof.

The prefix “Cᵤ-Cᵥ” indicates that the following group has from u to v carbon atoms. For example, “C₁-C₈ alkyl” indicates that the alkyl group has from 1 to 8 carbon atoms.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadcyl, hexadecyl, heptadecyl and octadecyl. The alkyl group can be monovalent or multivalent, e.g., divalent.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. The alkenyl group can be monovalent or multivalent, e.g., divalent.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₆ alkynyl), 2 to 6 carbon atoms (i.e., C₂₋₈ alkynyl), or 2 to 4 carbon atoms (i.e., C₂₋₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl. The alkynyl group can be monovalent or multivalent, e.g., divalent.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or —OEt), isopropoxy (—O—CH(CH₃)₂), t-butoxy (—O—C(CH₃)₃ or —OtBu) and the like. Other examples of suitable alkoxy groups include, but are not limited to, sec-butoxy, tert-butoxy, pentoxy, hexoxy, and the like. The alkoxy group can be monovalent or multivalent, e.g., divalent.

“Alkoxyalkyl” refers an alkoxy group linked to an alkyl group which is linked to the remainder of the compound. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). Alkoxy and alkyl are as defined above. Examples of “alkoxyalkyl” include, but are not limited to, methoxymethyl (CH₃OCH₂—), and methoxyethyl (CH₃OCH₂CH₂). The alkoxyalkyl group can be monovalent or multivalent, e.g., divalent.

“Bridged” means a ring system in which non-adjacent atoms on a ring are connected by a divalent substituent, such as an alkylenyl or heteroalkylenyl group or a single heteroatom.

“Hydroxyalkyl” refers to a hydroxy group, —OH, linked to an alkyl group which is linked to the remainder of the compound such that the alkyl group is divalent. Hydroxyalkyl can have any suitable number of carbons, such as from 1 to 8 (C₁₋ₛ hydroxyalkyl), 1 to 6 (C₁₋₆ hydroxyalkyl), 2 to 6 (C₂₋₆ hydroxyalkyl), 2 to 4 (C₂₋₄ hydroxyalkyl), or 2 to 3 (C₂₋₃ hydroxyalkyl). Alkyl is as defined above where the alkyl is divalent.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₈ haloalkyl), 1 to 6 carbon atoms (i.e., C₁-C₆ alkyl) or 1 to 3 carbon atoms (i.e., C₁-C₃ alkyl). The alkyl groups can be substituted with 1, 2, 3, 4, 5, 6, 7, 8, 9 or more halogens. Examples of suitable haloalkyl groups include, but are not limited to, —CF₃, —CHF₂, —CFH₂, —CH₂CF₃, fluorochloromethyl, difluorochloromethyl, and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, 4, 5, 6, 7, 8, 9 or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Thioalkyl” refers to a thio group, —SH, linked to an alkyl group which is linked to the remainder of the compound such that the alkyl group is divalent. Thioalkyl can have any suitable number of carbons, such as from 1 to 8 (C₁₋₈ thioalkyl), 1 to 6 (C₁₋₆ thioalkyl), 2 to 6 (C₂₋₆ thioalkyl), 2 to 4 (C₂₋₄ thioalkyl), or 2 to 3 (C₂₋₃ thioalkyl). Alkyl is as defined above where the alkyl is divalent.

“Thiohaloalkyl” is a thioalkyl group, as defined above, in which one or more hydrogen atoms of the alkyl group is replaced with a halogen atom. The alkyl portion of a thiohaloalkyl group can have 1 to 20 carbon atoms (i.e., C₁-C₂₀ thiohaloalkyl), 1 to 12 carbon atoms (i.e., C₁-C₁₂ thiohaloalkyl), 1 to 8 carbon atoms (i.e., C₁-C₈ thiohaloalkyl), 1 to 6 carbon atoms (i.e., C₁-C₆ thiohaloalkyl) or 1 to 3 carbon atoms (i.e., C₁-C₃ thiohaloalkyl). The thiohaloalkyl groups can be substituted with 1, 2, 3, 4, 5, 6, 7, 8, 9 or more halogens. Alkyl is as defined above where the alkyl is divalent.

“Cyanoalkyl” refers to a cyano group, —CN, linked to an alkyl group which is linked to the remainder of the compound such that the alkyl group is divalent. Cyanoalkyl can have any suitable number of carbons, such as from 1 to 8 (C₁₋₈ cyanoalkyl), 1 to 6 (C₁₋₆ cyanoalkyl), 2 to 6 (C₂₋₆ cyanoalkyl), 2 to 4 (C₂₋₄ cyanoalkyl), or 2 to 3 (C₂₋₃ cyanoalkyl). Alkyl is as defined above where the alkyl is divalent.

“Cycloalkyl” refers to a saturated or partially saturated cyclic alkyl group having a single ring or multiple rings, such as 2, 3, 4 or more, wherein the multiple rings can be fused, bridged, spiro, or any combination thereof. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

The term “fused” refers to a ring system in which two or more rings in the system share a pair of adjacent ring atoms.

“Spiro” refers to at least two rings are linked together by one common atom. “Spiro” also refers to a ring substituent which is joined by two bonds at the same carbon atom. Examples of spiro groups include, but are not limited to, 1,1-diethylcyclopentane, dimethyl-dioxolane, and 4-benzyl-4-methylpiperidine, wherein the cyclopentane and piperidine, respectively, are the spiro substituents.

“Alkyl-cycloalkyl” refers to a radical having an alkyl component and a cycloalkyl component, where the alkyl component links the cycloalkyl component to the point of attachment.

The alkyl component is as defined above, except that the alkyl component is at least divalent, e.g., an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycle” or “heterocyclyl” or “heterocycloalkyl” refer to a saturated or partially saturated cyclic alkyl group, with one or more ring heteroatoms independently selected from nitrogen, oxygen, sulfur and silicon. A heterocyclyl can be a single ring or multiple rings, such as 2, 3, 4 or more, wherein the multiple rings can be fused, bridged, spiro, or any combination thereof. For instance, heterocyclyl includes, without limitation, fused rings indoline, isoindoline, 4,5,6,7-tetrahydro-2H-pyrazolo[3,4-c]pyridine, and 2,3-dihydro-1H-benzo[d]imidazole; and spiro rings 2-azaspiro[3.3]heptane, 2-azaspiro[3.5]nonane, and 7-azaspiro[3.5]nonane. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, piperazinyl, oxetanyl, dioxolanyl, azetidinyl, and morpholinyl.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, e.g., an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Alkyl-aryl” refers to a radical having an alkyl component and an aryl component, where the alkyl component links the aryl component to the point of attachment. The alkyl component is as defined above, except that the alkyl component is at least divalent, e.g., an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, e.g., an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within.

“KRAS G12C” refers to the G12C mutation of the KRAS protein, where cysteine replaces glycine at amino acid position 12.

“KRAS G12C inhibitor” refers to compounds of the present disclosure, including compounds of Formula (I). The compounds modulate or inhibit some or all of the activity of KRAS G12C.

“KRAS G12C-associated disease or disorder” refers to diseases or disorders associated with or mediated by or having a KRAS G12C mutation. Representative diseases or disorders include, but are not limited to, KRAS G12C-associated cancer.

“KRAS G12D” refers to the G12D mutation of the KRAS protein, where aspartic acid replaces glycine at amino acid position 12.

“KRAS G12D inhibitor” refers to compounds of the present disclosure, including compounds of Formula (I). The compounds modulate or inhibit some or all of the activity of KRAS G12D.

“KRAS G12D-associated disease or disorder” refers to diseases or disorders associated with or mediated by or having a KRAS G12D mutation. Representative diseases or disorders include, but are not limited to, KRAS G12D-associated cancer.

“KRAS G12V” refers to the G12V mutation of the KRAS protein, where aspartic acid replaces valine at amino acid position 12.

“KRAS G12V inhibitor” refers to compounds of the present disclosure, including compounds of Formula (I). The compounds modulate or inhibit some or all of the activity of KRAS G12V.

“KRAS G12V-associated disease or disorder” refers to diseases or disorders associated with or mediated by or having a KRAS G12V mutation. Representative diseases or disorders include, but are not limited to, KRAS G12V-associated cancer.

A “KRAS degrader” refers to any agent that is capable of binding to and inducing the degradation of KRAS protein. Generally, protein degraders are believed to induce targeted protein degradation through recruitment of the cellular ubiquitinylation and proteasomal protein degradation machinery.

“KRAS inhibitor” and “KRAS modulator” are used interchangeably to refer to a compound disclosed herein capable of inhibiting, modulating, and/or degrading a KRAS protein (mutant or wild-type) in an assay indicative of KRAS inhibition efficacy. “KRAS inhibition” or “KRAS modulation” includes modulation or promotion of degradation of a KRAS protein (mutant or wild-type).

“Oxo” refers to the group (═O) or (O).

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule.

As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), (Ia), and/or (Ib) can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond.

Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. Similarly, an “arylalkyl” group, for example, can be attached to the remainder of the molecule at either an aryl or an alkyl portion of the group. A prefix such as “Cᵤ-Cᵥ” or “(Cᵤ-Cᵥ)” indicates that the following group has from u to v carbon atoms. For example, “C₁₋₆ alkyl” and “C₁-C₆ alkyl” both indicate that the alkyl group has from 1 to 6 carbon atoms.

Unless otherwise specified, the carbon atoms of the compounds of Formula (I) are intended to have a valence of four. If, in some chemical structure representations, carbon atoms do not have a sufficient number of variables attached to produce a valence of four, the remaining carbon substituents needed to provide a valence of four should be assumed to be hydrogen.

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein. The term “therapeutically effective amount” or “effective amount” also means amounts that eliminate or reduce the subject's viral burden and/or viral reservoir.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

“Subject” refers to animals such as mammals, including, but not limited to, primates (e.g., humans), cows, sheep, goats, horses, dogs, cats, rabbits, rats, mice and the like. In some embodiments, the subject is a human.

“Disease” or “condition” refer to a state of being or health status of a patient or subject capable of being treated with a compound, pharmaceutical composition, or method provided herein. The disease may be an autoimmune, inflammatory, cancer, infectious (e.g., a viral infection), metabolic, developmental, cardiovascular, liver, intestinal, endocrine, neurological, or other disease. In some embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

“Cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma.

Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

“Leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

“Sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

“Melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

“Carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basal oid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

“Metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) is caused by (in whole or in part), or a symptom of the disease is caused by (in whole or in part) the substance or substance activity or function.

The term “adjacent carbons” as used herein refers to consecutive carbons atoms that are directly attached to each other. For example,

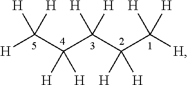

C₁ and C₂ are adjacent carbons, C₂ and C₃ are adjacent carbons, C₃ and C₄ are adjacent carbons, and C₄ and C₅ are adjacent carbons. Similarly, in

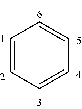

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

### III. Compounds

Disclosed herein are, among other things, compounds of Formula (I), (Ia), and/or (Ib). In some embodiments, the present disclosure provides a compound of Formula (I):

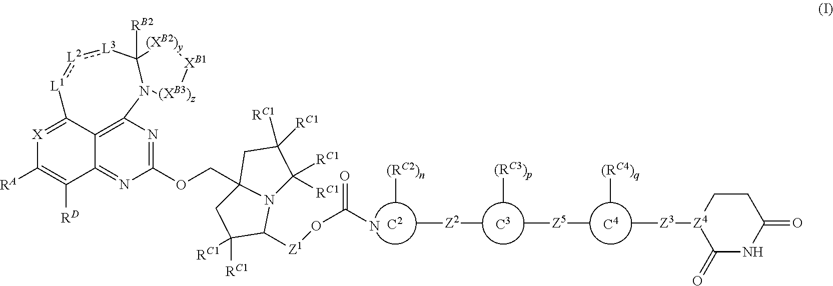

or a pharmaceutically acceptable salt thereof,
wherein
each R^{C1} is independently H, C₁-C₆ alkyl, C₂-C₆ alkoxyalkyl, C₁-C₆ hydroxyalkyl, halo, C₁-C₆ haloalkyl, C₁-C₆ haloalkoxy, —OH, —CN, C₁-C₆ cyanoalkyl, C₃-C₆ cycloalkyl, ═CH₂, ═CHF, or ═CF₂, or
alternatively, two R^{C1} attached to the same carbon form a C₃-C₆-membered cycloalkyl or 4- to 6-membered heterocyclyl, wherein the cycloalkyl and heterocyclyl is substituted by 0, 1, or 2 halo;
Z¹ is —(CR^{2f}R^{2g})ₘ—;
R^{2f} and R^{2g} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2f} and R^{2g} can combine with the carbon to which they are attached to form a C³-C⁶ cycloalkyl;
m is 1, 2, or 3;
ring C2 is 4- to 16-membered heterocyclyl;
each R^{C2} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, —CN, C₁-C₆ cyanoalkyl, or C₃-C₆-cycloalkyl;
n is 0, 1, 2, 3, or 4;
Z² is C₁-C₃ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z2b}—, —C(O)—, —S(O)—, S(O)₂—, S(O)(═NR^{Z2})—, oxetanyl, or a bond; wherein the alkyl and cycloalkyl is substituted with 0, 1, 2, 3, or 4 Z^{2a}; wherein the alkenyl is substituted with 0, 1, or 2 Z^{2c};
each Z^{2a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
each Z^{2c} is independently C₁-C₆ alkyl, halo, C₁-C₆ haloalkyl, or C₃-C₆ cycloalkyl;
R^{Z2} is H, C₁-C₆ alkyl, C₁-C₆ haloalkyl, or C₃-C₆ cycloalkyl;
R^{Z2b} is H, C₁-C₆ alkyl, C₁-C₆ haloalkyl, or C₃-C₆ cycloalkyl;
ring C3 is C₃-C₅ cycloalkyl, 4- to 16-membered heterocyclyl, C₆-C₁₀ aryl, 5- to 10-membered heteroaryl, or absent;
each R^{C3} is independently C₁-C₆ alkyl, C₂-C₆ alkenyl, C₂-C₆ alkynyl, halo, C₁-C₆ haloalkyl, C₁-C₆ haloalkoxy, C₁-C₆ thioalkyl, C₁-C₆ thiohaloalkyl, —OH, oxo, —CN, C₁-C₆ alkoxy, C₂-C₆ alkoxyalkyl, C₁-C₆ hydroxyalkyl, C₁-C₆ cyanoalkyl, or C₃-C₆-cycloalkyl;
p is 0, 1, 2, 3, or 4;
Z⁵ is C₁-C₃ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z5b}—, —C(O)—, —S(O)—, S(O)₂—, S(O)(═NR^{Z5})—, or a bond; wherein the alkyl and cycloalkyl is substituted with 0, 1, 2, 3, or 4 Z^{5a};
each Z^{5a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, —CN, C₁-C₆ cyanoalkyl, C₃-C₆ cycloalkyl, or 4- to 10-membered heterocyclyl;
R^{Z5} is H, C₁-C₆ alkyl, C₁-C₆ haloalkyl or C₃-C₆ cycloalkyl;
R^{Z5b} is H, C₁-C₆ alkyl, C₁-C₆ haloalkyl, or C₃-C₆ cycloalkyl;
ring C4 is 5 to 14-membered heterocyclyl, C₆-C₁₀ aryl, or 5- to 10-membered heteroaryl;
Z³ is —NH—, —C(O)NH—, or a bond, and Z⁴ is —CH—;
alternatively, Z³ is a bond and Z⁴ is N;
each R^{C4} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, —CN, C₁-C₃ cyanoalkyl, C₃-C₆ cycloalkyl, or 4- to 10-membered heterocyclyl;
q is 0, 1, 2, 3, or 4;
X is N, CH, or CR^{x};
R^{x} is halo, C₁-C₃ haloalkyl, or C₁-C₃ cyanoalkyl;
L¹ is O, CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6-membered heterocyclyl having 1 heteroatom that is O, wherein each cycloalkyl and heterocyclyl is substituted with 0, 1, 2, or 3 R^{1x};
R^{1e} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
such that when L² is ═C(R^{2e})— then L¹ is —C(R^{1e})═ or L³ is ═C(R^{3e})—, and when L³ is ═C(R^{3e})— then L² is ═C(R^{2e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SR^{A2a}, —S(O)₂R^{A2a}, —S(O)₂OR^{A2a}, —N(R^{A2a})(R^{A2b}), —(C₀-C₃ alkyl)-SFs, C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, —SR^{A3a}—N(R^{A3a})(R^{A3b}) C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ thiohaloalkyl, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B1});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} attached to two different atoms can combine to form a C₃-C₁₀ cycloalkyl or 4- to 10-membered heterocyclyl, wherein the cycloalkyl and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
R^{D} is halo;
wherein, unless otherwise indicated, each heterocyclyl has 1, 2, 3, or 4 heteroatoms selected from N, O, S, and Si; and
each heteroaryl has 1, 2, 3, or 4 heteroatoms selected from N, O, and S.

In some embodiments, the present disclosure provides the compound of Formula (I), or a pharmaceutically acceptable salt thereof, wherein X^{B1} is O. In some embodiments, the present disclosure provides the compound of Formula (I), or a pharmaceutically acceptable salt thereof, wherein X^{B1} is CH₂.

In some embodiments, the present disclosure provides the compound of Formula (I), or a pharmaceutically acceptable salt thereof, wherein y is 1. In some embodiments, the present disclosure provides the compound of Formula (I), or a pharmaceutically acceptable salt thereof, wherein y is 2.

In some embodiments, the present disclosure provides the compound of Formula (I), or a pharmaceutically acceptable salt thereof, wherein X^{B2} is CH₂ or CF₂.

In some embodiments, the present disclosure provides the compound of Formula (I), or a pharmaceutically acceptable salt thereof, wherein X^{B3} is CH₂ or CF₂.

In some embodiments, the present disclosure provides the compound of Formula (I), or a pharmaceutically acceptable salt thereof, wherein z is 2. In some embodiments, the present disclosure provides the compound of Formula (I), or a pharmaceutically acceptable salt thereof, wherein z is 3.

In some embodiments, the present disclosure provides the compound of Formula (I), or a pharmaceutically acceptable salt thereof, wherein X^{B1} is CH₂ or O; X^{B2} and X^{B3} are each CH₂; y is 1 or 2; and z is 2 or 3.

In some embodiments, the present disclosure provides the compound of Formula (I), or a pharmaceutically acceptable salt thereof, wherein each R^{C1} is independently H, C₁-C₆ alkyl, halo, C₁-C₆ haloalkyl, ═CH₂, ═CHF, or ═CF₂, or alternatively, two R^{C1} attached to the same carbon form cyclopropyl substituted by 0, 1, or 2 halo.

In some embodiments, the present disclosure provides the compound of Formula (I), or a pharmaceutically acceptable salt thereof, having the structure of Formula (Ia):

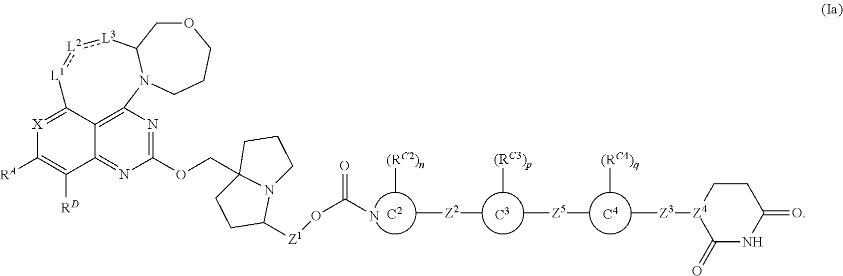

In some embodiments, the present disclosure provides the compound of Formula (I) and/or (Ia), or a pharmaceutically acceptable salt thereof, having the structure of Formula (Ib):

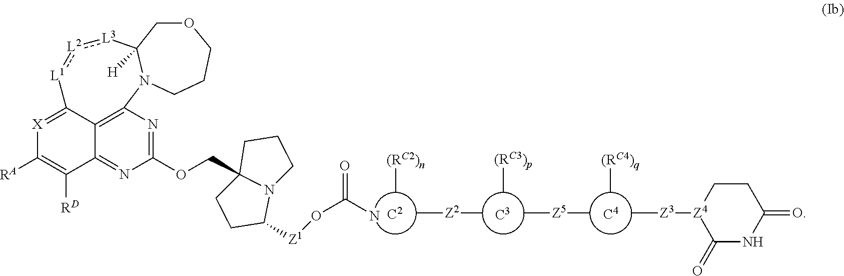

In some embodiments, the present disclosure provides the compound of Formula (I), (Ia), and/or (Ib), or a pharmaceutically acceptable salt thereof, wherein X is N. In some embodiments, the present disclosure provides the compound of Formula (I), (Ia), and/or (Ib), or a pharmaceutically acceptable salt thereof, wherein X is CH. In some embodiments, the present disclosure provides the compound of Formula (I), (Ia), and/or (Ib), or a pharmaceutically acceptable salt thereof, wherein X is C—F. In some embodiments, the present disclosure provides the compound of Formula (I), (Ia), and/or (Ib), or a pharmaceutically acceptable salt thereof, wherein X is C—Cl.

In some embodiments, the present disclosure provides the compound of Formula (I), (Ia), and/or (Ib), or a pharmaceutically acceptable salt thereof, wherein L¹ is O.

In some embodiments, the present disclosure provides the compound of Formula (I), (Ia), and/or (Ib), or a pharmaceutically acceptable salt thereof, wherein L¹ is CR^{1a}R^{1b} or —C(R^{1e})═. In some embodiments, the present disclosure provides the compound of Formula (I), (Ia), and/or (Ib), or a pharmaceutically acceptable salt thereof, wherein L¹ is O, S, or CR^{1a}R^{1b}. In some embodiments, the present disclosure provides the compound of Formula (I), (Ia), and/or (Ib), or a pharmaceutically acceptable salt thereof, wherein L¹ is CHR^{1b}.

In some embodiments, the present disclosure provides the compound of Formula (I), (Ia), and/or (Ib), or a pharmaceutically acceptable salt thereof, wherein R^{1a} and R^{1b} are each independently H, C₁-C₃ alkyl, or halo.

In some embodiments, the present disclosure provides the compound of Formula (I), (Ia), and/or (Ib), or a pharmaceutically acceptable salt thereof, wherein R^{1b} is C₁-C₃ alkyl or halo. In some embodiments, the present disclosure provides the compound of Formula (I), (Ia), and/or (Ib), or a pharmaceutically acceptable salt thereof, wherein R^{1b} is methyl. In some embodiments, the present disclosure provides the compound of Formula (I), (Ia), and/or (Ib), or a pharmaceutically acceptable salt thereof, wherein R^{1b} is H.

In some embodiments, the present disclosure provides the compound of Formula (I), (Ia), and/or (Ib), or a pharmaceutically acceptable salt thereof, wherein L¹ is CH₂, CHCH₃, CHCH₂CH₃, CHF, or CF₂.

In some embodiments, the present disclosure provides the compound of (I), (Ia), and/or (Ib), or a pharmaceutically acceptable salt thereof, wherein L¹ is CH₂.

In some embodiments, the present disclosure provides the compound of Formula (I), (Ia), and/or (Ib), or a pharmaceutically acceptable salt thereof, wherein R^{2a} and R^{2b} are each independently H, C₁-C₃ alkyl, halo, or C₁-C₆ haloalkyl.

In some embodiments, the present disclosure provides the compound of Formula (I), (Ia), and/or (Ib), or a pharmaceutically acceptable salt thereof, wherein R^{2a} is H.

In some embodiments, the present disclosure provides the compound of Formula (I), (Ia), and/or (Ib), or a pharmaceutically acceptable salt thereof, wherein L² is CHR^{2b}.

In some embodiments, the present disclosure provides the compound of Formula (I), (Ia), and/or (Ib), or a pharmaceutically acceptable salt thereof, wherein R^{2b} is H or C₁-C₃ alkyl. In some embodiments, the present disclosure provides the compound of Formula (I), (Ia), and/or (Ib), or a pharmaceutically acceptable salt thereof, wherein R^{2b} is H. In some embodiments, the present disclosure provides the compound of Formula (I), (Ia), and/or (Ib), or a pharmaceutically acceptable salt thereof, wherein R^{2b} is C₁-C₃ alkyl. In some embodiments, the present disclosure provides the compound of Formula (I), (Ia), and/or (Ib), or a pharmaceutically acceptable salt thereof, wherein R^{2b} is methyl.

In some embodiments, the present disclosure provides the compound of Formula (I), (Ia), and/or (Ib), or a pharmaceutically acceptable salt thereof, wherein L¹ is —C(R^{1e})═; and L² is ═C(R^{2e})—. In some embodiments, the present disclosure provides the compound of Formula (I), (Ia), and/or (Ib), or a pharmaceutically acceptable salt thereof, wherein L¹ is —C(CH₃)═; and L² is ═CH—.

In some embodiments, the present disclosure provides the compound of Formula (I), (Ia), and/or (Ib), or a pharmaceutically acceptable salt thereof, wherein L³ is a bond. In some embodiments, the present disclosure provides the compound of Formula (I), (Ia), and/or (Ib), or a pharmaceutically acceptable salt thereof, wherein L³ is CR^{3a}R^{3b}.

In some embodiments, the present disclosure provides the compound of Formula (I), (Ia), and/or (Ib), or a pharmaceutically acceptable salt thereof, wherein R^{3a} and R^{3b} are H.

In some embodiments, the present disclosure provides the compound of Formula (I), (Ia), and/or (Ib), or a pharmaceutically acceptable salt thereof, wherein L³ is CH₂ or CHCH₃.

In some embodiments, the present disclosure provides the compound of Formula (I), (Ia), and/or (Ib), or a pharmaceutically acceptable salt thereof, wherein L¹ is CR^{1a}R^{1b}; L² is a bond, CR^{2a}R^{2b}, O, or S; and L³ is a bond or CR^{3a}R^{3b}. In some embodiments, the present disclosure provides the compound of Formula (I), (Ia), and/or (Ib), or a pharmaceutically acceptable salt thereof, wherein R^{1a} and R^{1b} are each independently H, C₁-C₃ alkyl, or halo. In some embodiments, the present disclosure provides the compound of Formula (I), (Ia), and/or (Ib), or a pharmaceutically acceptable salt thereof, wherein L¹ is CHR^{1b}. In some embodiments, the present disclosure provides the compound of Formula (I), (Ia), and/or (Ib), or a pharmaceutically acceptable salt thereof, wherein R^{1b} is C₁-C₃ alkyl, halo, or —OH. In some embodiments, the present disclosure provides the compound of Formula (I), (Ia), and/or (Ib), or a pharmaceutically acceptable salt thereof, wherein R^{1b} is C₁-C₃ alkyl or halo. In some embodiments, the present disclosure provides the compound of Formula (I), (Ia), and/or (Ib), or a pharmaceutically acceptable salt thereof, wherein R^{1b} is methyl, F, or —OH. In some embodiments, the present disclosure provides the compound of Formula (I), (Ia), and/or (Ib), or a pharmaceutically acceptable salt thereof, wherein R^{1b} is methyl. In some embodiments, the present disclosure provides the compound of Formula (I), (Ia), and/or (Ib), or a pharmaceutically acceptable salt thereof, wherein L¹ is CH₂.

In some embodiments, the present disclosure provides the compound of Formula (I), (Ia), and/or (Ib), or a pharmaceutically acceptable salt thereof, wherein R^{A} is phenyl substituted with 0, 1, or 2 R^{A2}. In some embodiments, the present disclosure provides the compound of Formula (I), (Ia), and/or (Ib), or a pharmaceutically acceptable salt thereof, wherein R^{A} is phenyl substituted with 3 R^{A2}. In some embodiments, the present disclosure provides the compound of Formula (I), (Ia), and/or (Ib), or a pharmaceutically acceptable salt thereof, wherein R^{A} is naphthyl substituted with 0, 1, 2, 3, 4, or 5 R^{A2}. In some embodiments, the present disclosure provides the compound of Formula (I), (Ia), and/or (Ib), or a pharmaceutically acceptable salt thereof, wherein R^{A} is naphthyl substituted with 2, 3, or 4 R^{A2}. In some embodiments, the present disclosure provides the compound of Formula (I), (Ia), and/or (Ib), or a pharmaceutically acceptable salt thereof, wherein R^{A} is 5- to 14-membered heteroaryl, wherein R^{A} is substituted with 0, 1, 2, 3, 4, or 5 R^{A2}. In some embodiments, the present disclosure provides the compound of Formula (I), (Ia), and/or (Ib), or a pharmaceutically acceptable salt thereof, wherein R^{A} is 5- to 6-membered heteroaryl, wherein R^{A} is substituted with 0, 1, or 2 R^{A2}. In some embodiments, the present disclosure provides the compound of Formula (I), (Ia), and/or (Ib), or a pharmaceutically acceptable salt thereof, wherein R^{A} is pyridyl, wherein R^{A} is substituted with 0, 1, or 2 R^{A2}.

In some embodiments, the present disclosure provides the compound of Formula (I), (Ia), and/or (Ib), or a pharmaceutically acceptable salt thereof, wherein each R^{A2} is independently C₁-C₆ alkyl, —OH, C₂-C₆ alkenyl, C₂-C₆ alkynyl, halo, C₁-C₆ haloalkyl, —CN, oxo, —OR^{A2a}, —SR^{A2a}, —N(R^{A2a})(R^{A2b}), or —(C₁-C₆ alkyl)-(C₃-C₈ cycloalkyl), wherein each alkenyl is substituted with 0, 1, 2, or 3 R^{A3}; each R^{A2a} and R^{A2b} is independently H, C₁-C₆ haloalkyl, or C₃-C₈ cycloalkyl; and each R^{A3} is independently halo.

In some embodiments, the present disclosure provides the compound of Formula (I), (Ia), and/or (Ib), or a pharmaceutically acceptable salt thereof, wherein each R^{A2} is independently Me, Et, —OH, —CH═CHMe, —C(Cl)═CH₂, —CH≡CHF₂, —C≡CH, F, Cl, CF₃, —CH₂CF₃, —CN, oxo, —OCF₃, —O-cyclopropyl, —SCF₃, —NH₂, or —CH₂-cyclopropyl. In some embodiments, the present disclosure provides the compound of Formula (I), (Ia), and/or (Ib), or a pharmaceutically acceptable salt thereof, wherein each R^{A2} is independently —C≡CH, F, Cl, CF₃, —CN, or —NH₂.

In some embodiments, the present disclosure provides the compound of Formula (I), (Ia), and/or (Ib), or a pharmaceutically acceptable salt thereof, wherein each R^{A2} is independently C₁-C₆ alkyl, —OH, C₂-C₆ alkenyl, C₂-C₆ alkynyl, halo, C₁-C₆ haloalkyl, —OR^{A2a}, —SR^{A2a}, or —(C₁-C₆ alkyl)-(C₃-C₈ cycloalkyl), wherein each alkenyl is substituted with 0, 1, 2, or 3 R^{A3}; each R^{A2a} and R^{A2b} is independently H, C₁-C₆ haloalkyl, or C₃-C₈ cycloalkyl; and each R^{A3} is independently halo.

In some embodiments, each R^{A2} is independently Me, —OH, —C(Cl)═CH₂, —CH═CHF₂, —C≡CH, F, Cl, —CH₂CF₃, —OCF₃, —O-cyclopropyl, —SCF₃, or —CH₂-cyclopropyl.

In some embodiments, the present disclosure provides the compound of Formula (I), (Ia), and/or (Ib), or a pharmaceutically acceptable salt thereof, wherein R^{A} is

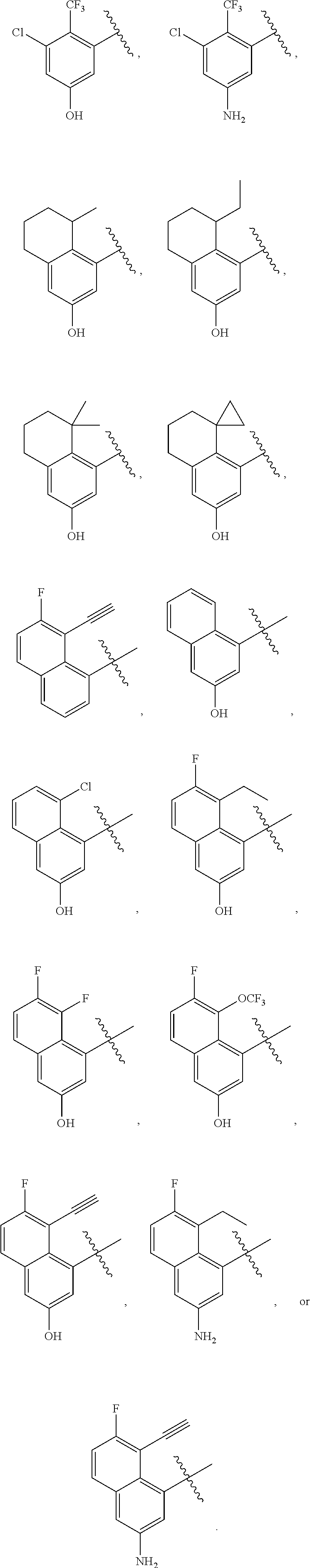

In some embodiments, the present disclosure provides the compound of Formula (I), (Ia), and/or (Ib), or a pharmaceutically acceptable salt thereof, wherein R^{A} is

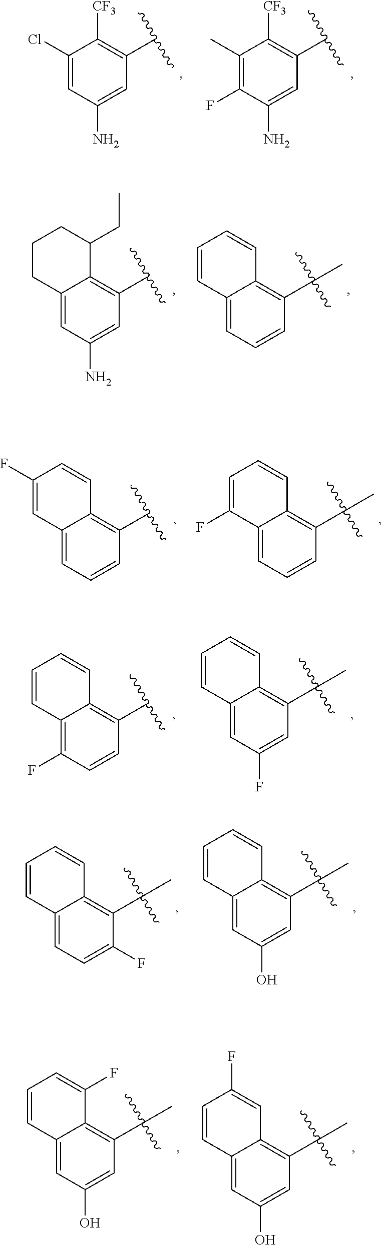

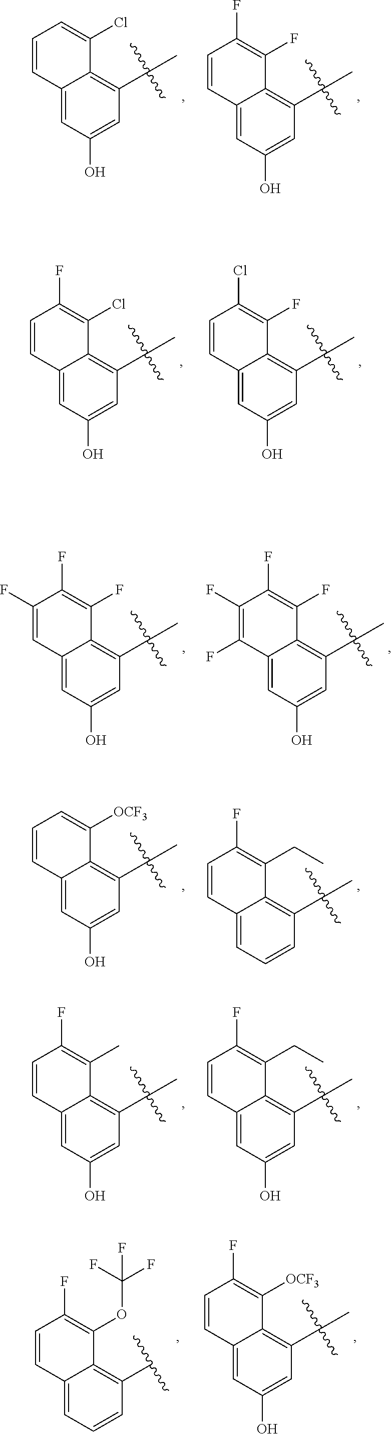

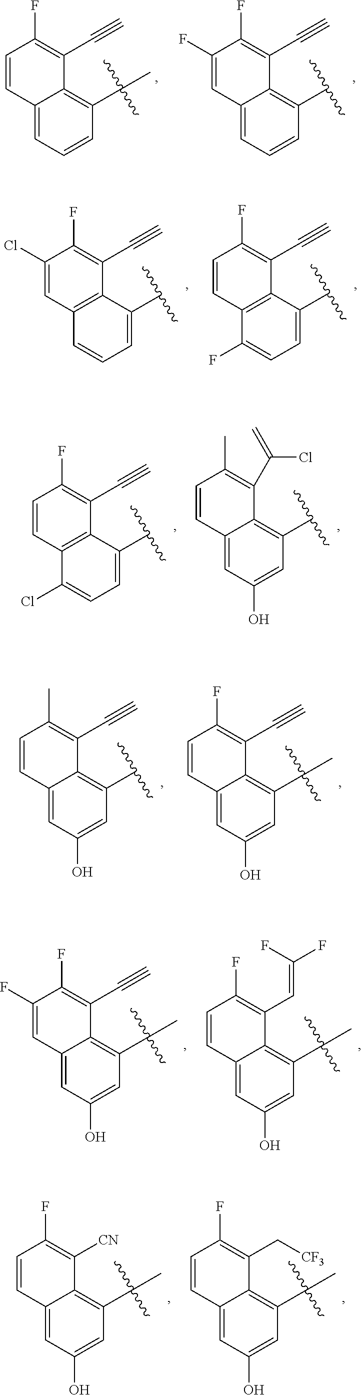

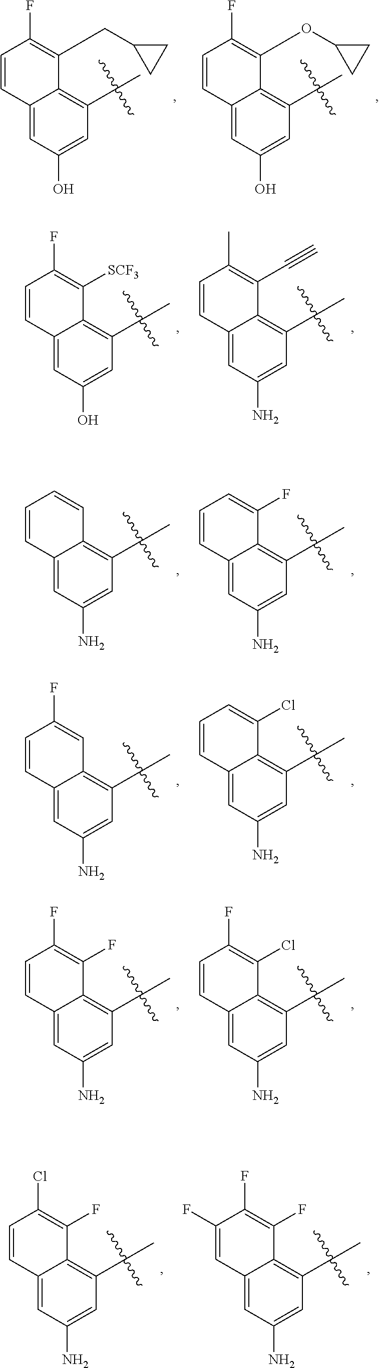

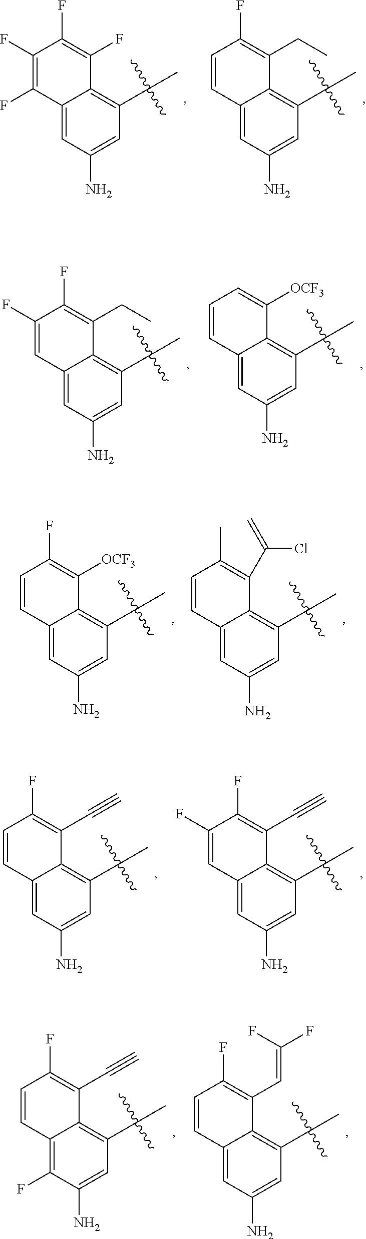

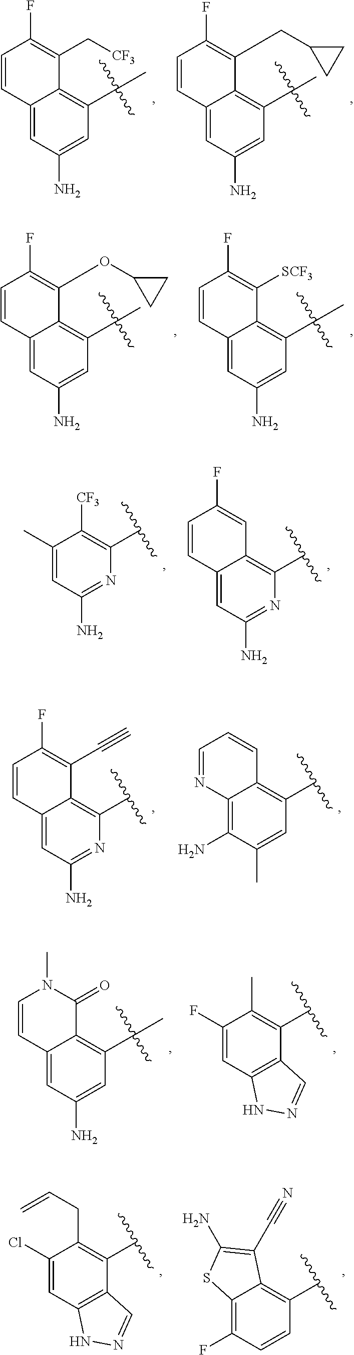

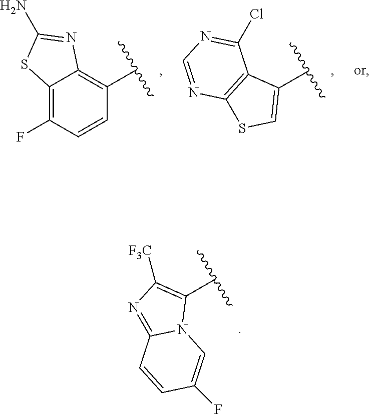 [SMILES: *c1ccc(F)c2sc(N)nc12 ; *c1csc2ncnc(Cl)c12 ; *c1c(C(F)(F)F)nc2ccc(F)cn12]

In some embodiments, the present disclosure provides the compound of Formula (I), (Ia), and/or (Ib), or a pharmaceutically acceptable salt thereof, wherein R^{A} is

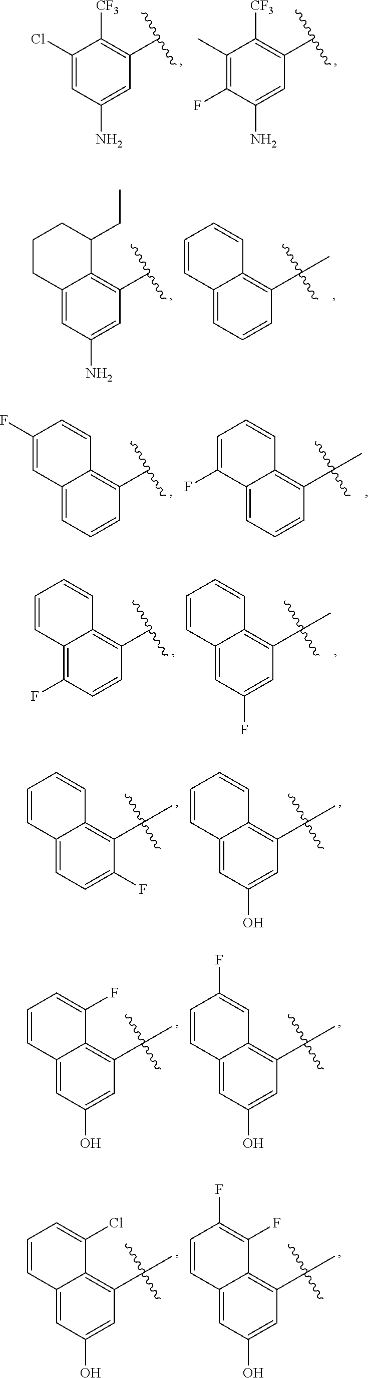

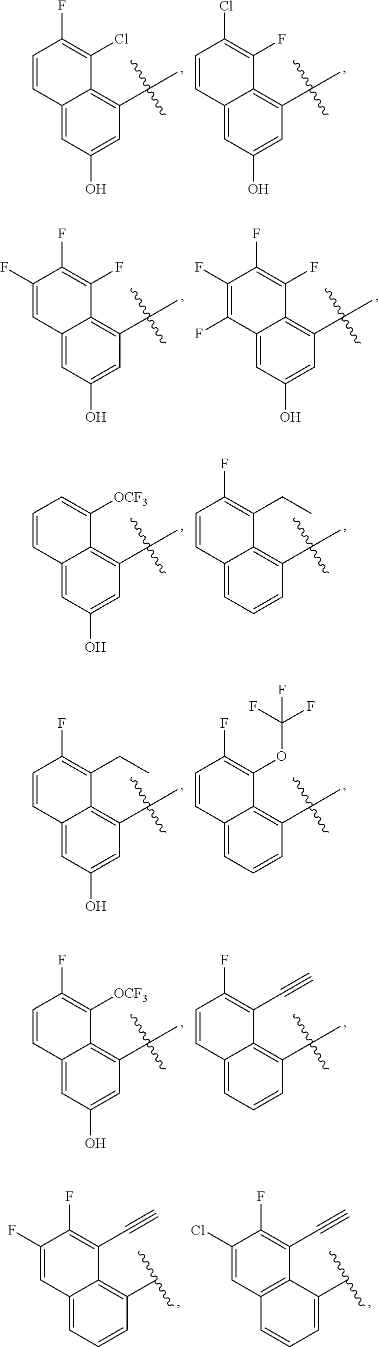

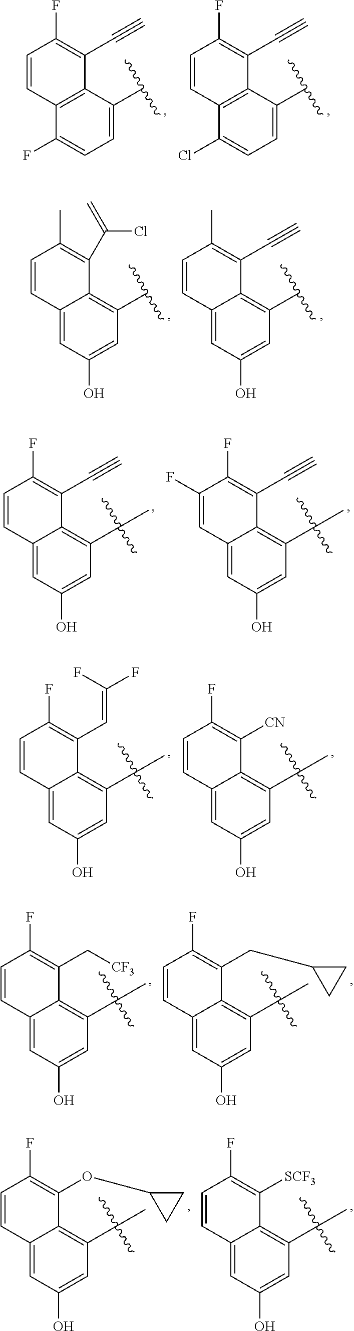

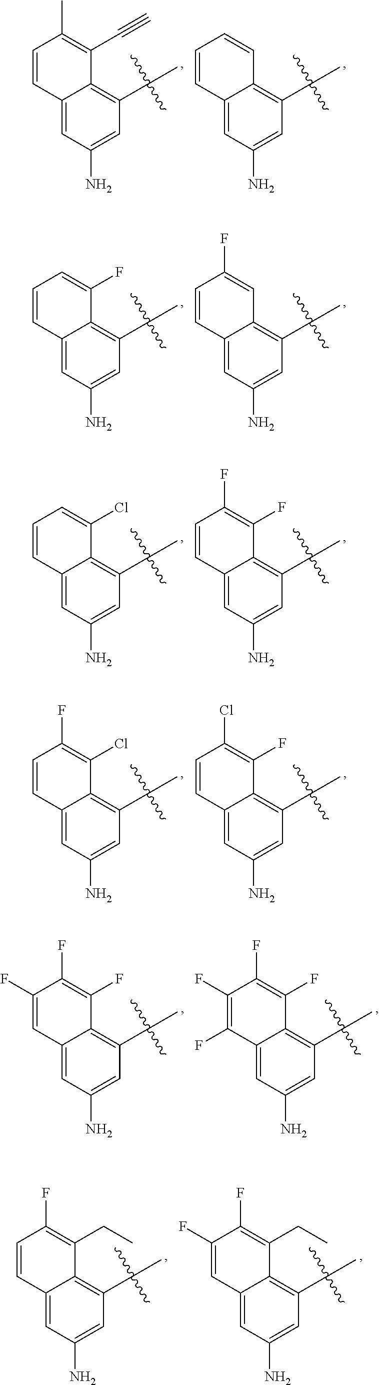

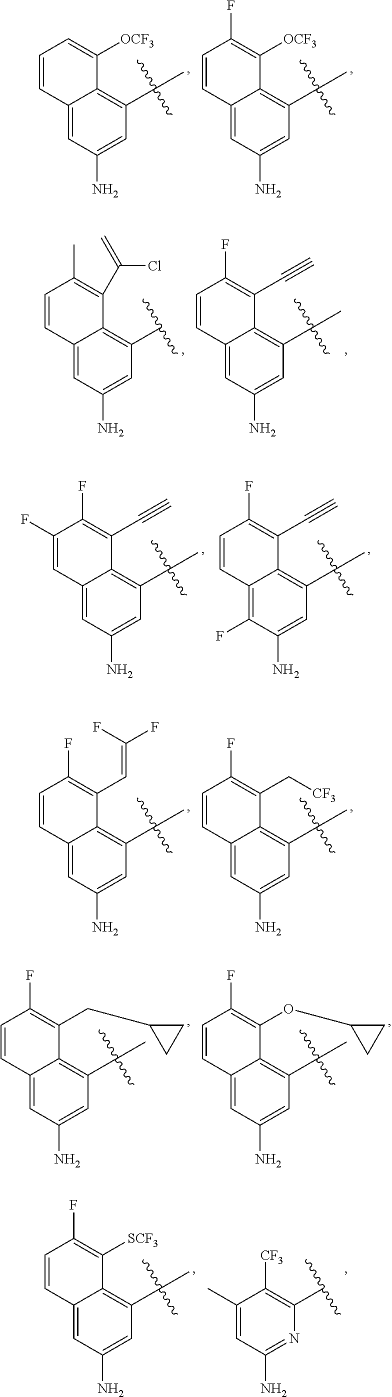

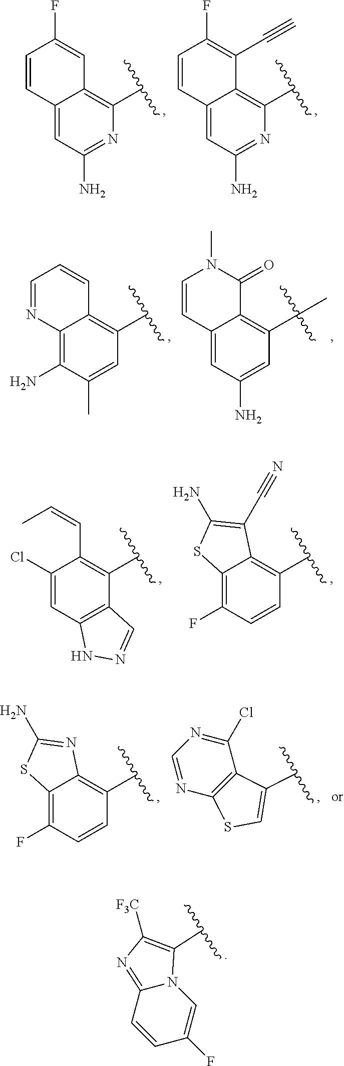

In some embodiments, the present disclosure provides the compound of Formula (I), (Ia), and/or (Ib), or a pharmaceutically acceptable salt thereof, wherein R^{A} is

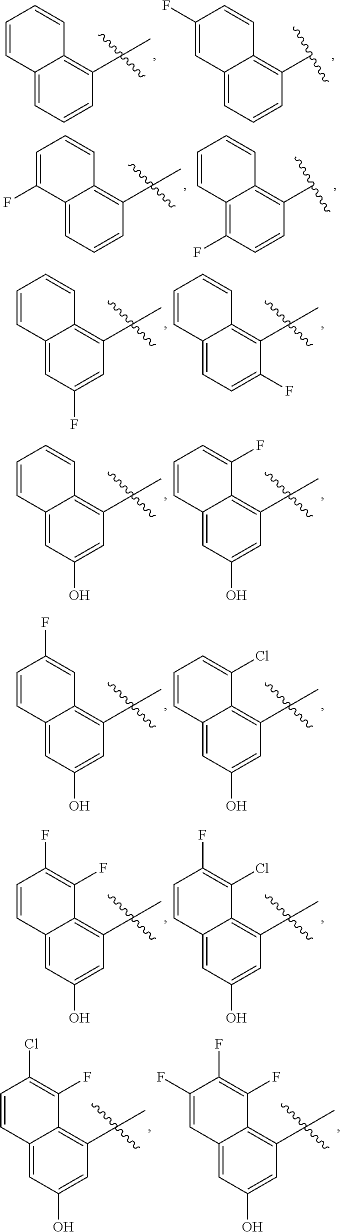

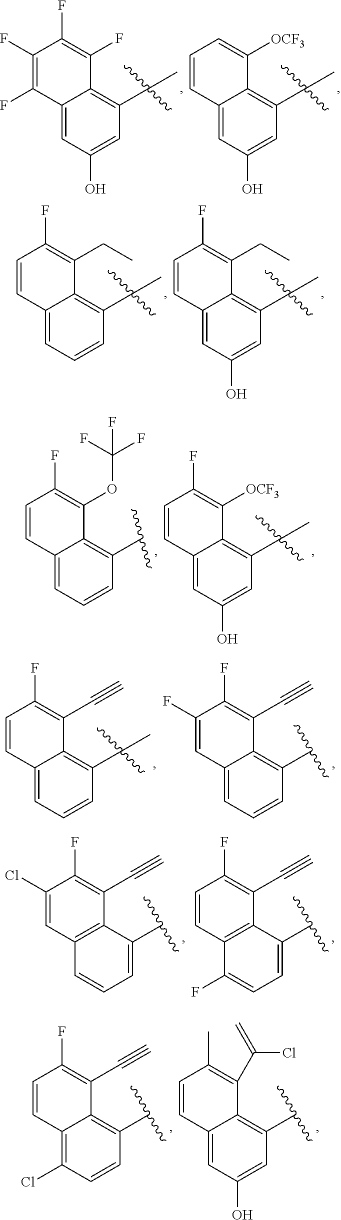

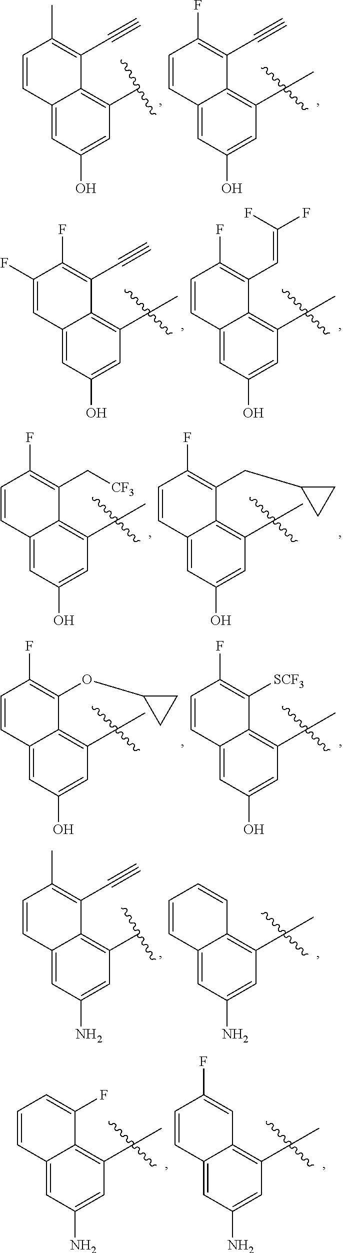

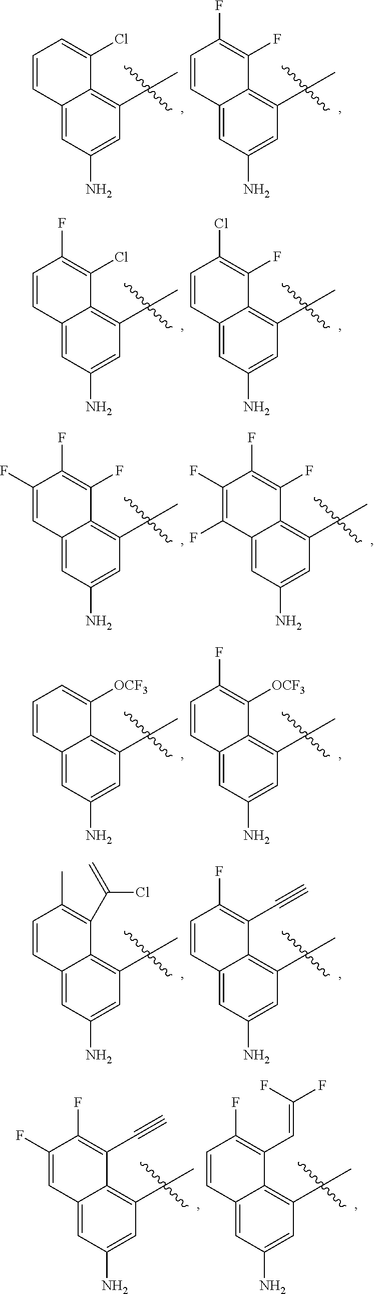

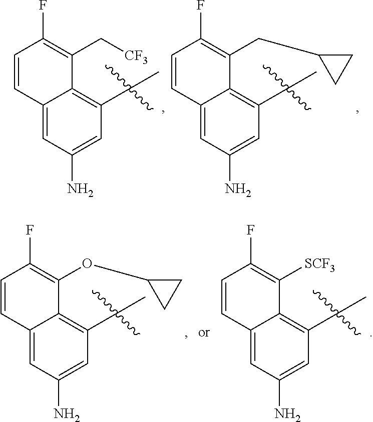

In some embodiments, the present disclosure provides the compound of Formula (I), (Ia), and/or (Ib), or a pharmaceutically acceptable salt thereof, wherein R^{A} is

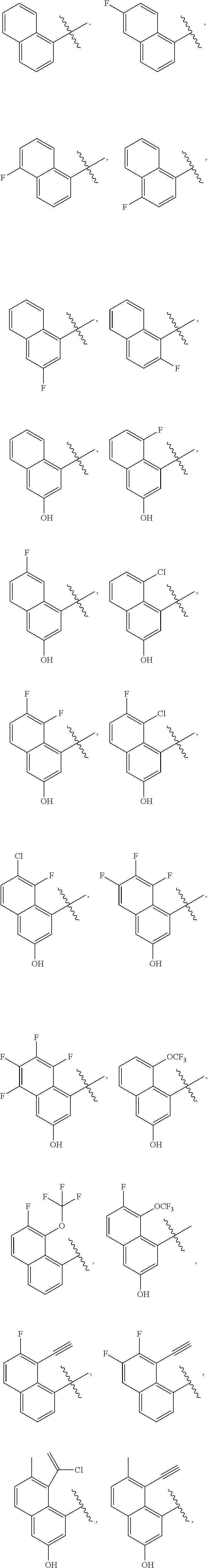

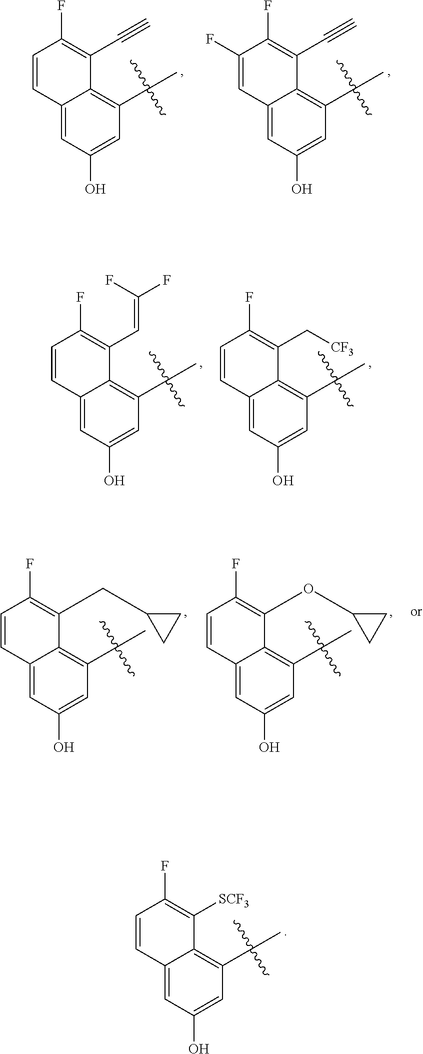

In some embodiments, the present disclosure provides the compound of Formula (I), (Ia), and/or (Ib), or a pharmaceutically acceptable salt thereof, wherein R^{A} is

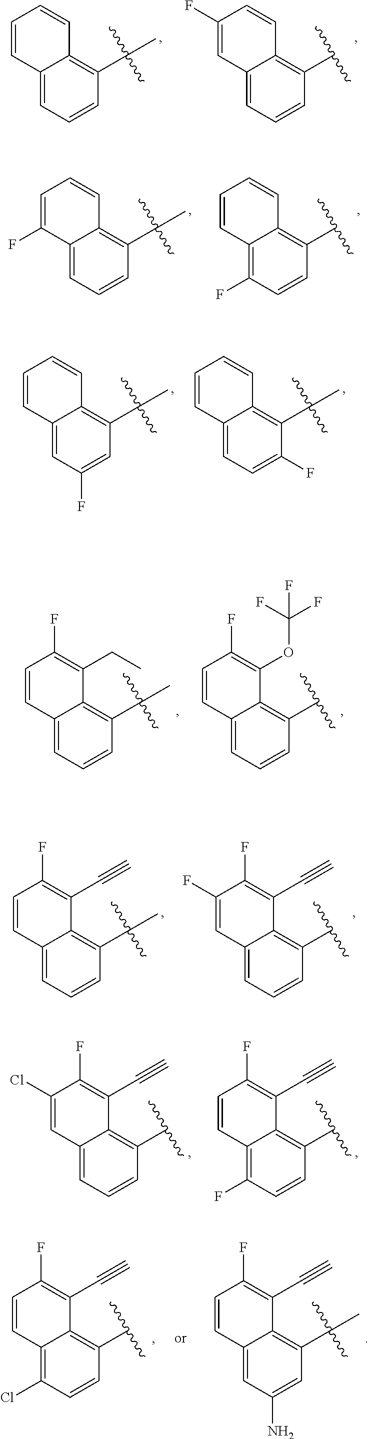

In some embodiments, the present disclosure provides the compound of Formula (I), (Ia), and/or (Ib), or a pharmaceutically acceptable salt thereof, wherein R^{A} is

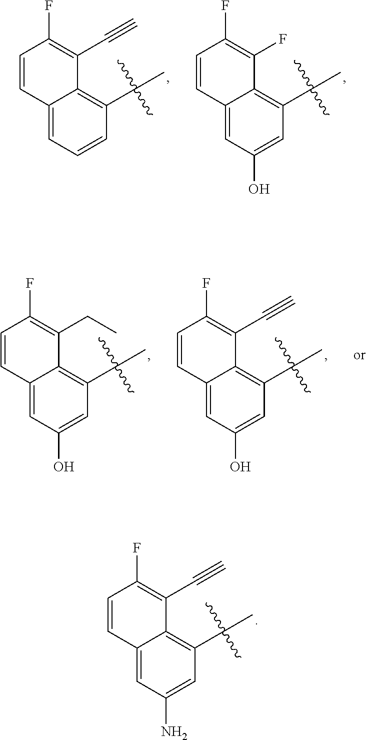 [SMILES: *c1cccc2ccc(F)c(C#C)c12 ; *c1cc(O)cc2ccc(F)c(F)c12 ; *c1cc(O)cc2ccc(F)c(CC)c12 ; *c1cc(O)cc2ccc(F)c(C#C)c12 ; *c1cc(N)cc2ccc(F)c(C#C)c12]

In some embodiments, the present disclosure provides the compound of Formula (I), (Ia), and/or (Ib), or a pharmaceutically acceptable salt thereof, wherein R^{A} is

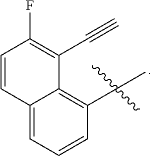 [SMILES: *c1cccc2ccc(F)c(C#C)c12]

In some embodiments, the present disclosure provides the compound of Formula (I), (Ia), and/or (Ib), or a pharmaceutically acceptable salt thereof, wherein L¹ is O or CR^{1a}R^{1b}; R^{1a} and R^{1b} are each independently H, C₁-C₃ alkyl, or halo; L² is CR^{2a}R^{2b}; R^{2a} and R^{2b} are each independently H, C₁-C₃ alkyl, C₁-C₆ haloalkyl, or C₃-C₆ cycloalkyl; L³ is a bond or CR^{3a}R^{3b}; R^{3a} and R^{3b} are each independently H or C₁-C₃ alkyl; R^{A} is naphthyl, wherein R^{A} is substituted with 0, 1, 2, 3, 4, or 5 R^{A2}; each R^{A2} is independently —OH, C₁-C₃ alkyl, C₂-C₆ alkenyl, C₂-C₆ alkynyl, C₁-C₆ alkoxy, C₁-C₆ thioalkyl, halo, C₁-C₆ haloalkyl, —OR^{A2a}, —SR^{A2a}, —N(R^{A2a})(R^{A2b}), or —(C₁-C₆ alkyl)-(C₃-C₈ cycloalkyl), wherein each alkyl, alkenyl, alkynyl, alkoxy, and haloalkyl is substituted with 0, 1, 2, or 3 R^{A3}; each R^{A2a} and R^{A2b} is independently H, C₁-C₆ haloalkyl, or C₃-C₈ cycloalkyl; and each R^{A3} is independently halo.

In some embodiments, the present disclosure provides the compound of Formula (I), (Ia), and/or (Ib), or a pharmaceutically acceptable salt thereof, wherein X is N, CH, or CR^{x}; R^{x} is halo; L¹ is O or CR^{1a}R^{1b}; R^{1a} and R^{1b} are each independently H, C₁-C₃ alkyl, or halo; L² is CR^{2a}R^{2b}; R^{2a} and R^{2b} are each independently H, C₁-C₃ alkyl, C₁-C₆ haloalkyl, or C₃-C₆ cycloalkyl; L³ is a bond or CR^{3a}R^{3b}; R^{3a} and R^{3b} are each independently H or C₁-C₃ alkyl; R¹ is H or F; R² is F; R³, R⁴, and R⁵ are each H; R^{A} is naphthyl, wherein R^{A} is substituted with 0, 1, 2, 3, 4, or 5 R^{A2}; each R^{A2} is independently —OH, C₁-C₃ alkyl, C₂-C₆ alkenyl, C₂-C₆ alkynyl, C₁-C₆ alkoxy, C₁-C₆ thioalkyl, halo, C₁-C₆ haloalkyl, —OR^{A2a}, —SR^{A2a}, —N(R^{A2a})(R^{A2b}), or —(C₁-C₆ alkyl)-(C₃-C₈ cycloalkyl), wherein each alkyl, alkenyl, alkynyl, alkoxy, and haloalkyl is substituted with 0, 1, 2, or 3 R^{A3}; each R^{A2a} and R^{A2b} is independently H, C₁-C₆ haloalkyl, or C₃-C₈ cycloalkyl; each R^{A3} is independently halo; and R^{D} is F.

In some embodiments, the present disclosure provides the compound of Formula (I), (Ia), and/or (Ib), or a pharmaceutically acceptable salt thereof, wherein X is N, CH, or C—Cl; L¹ is O, CH₂, CHCH₃, CHCH₂CH₃, CHF, or CF₂; L² is CH₂, CHCH₃, CHCH₂CH₃, CHCHF₂, or

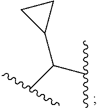 [SMILES: *C(*)C1CC1]

L³ is a bond, CH₂, or CHCH₃; R¹ is H or F; R² is F; R³, R⁴, and R⁵ are each H; R^{A} is

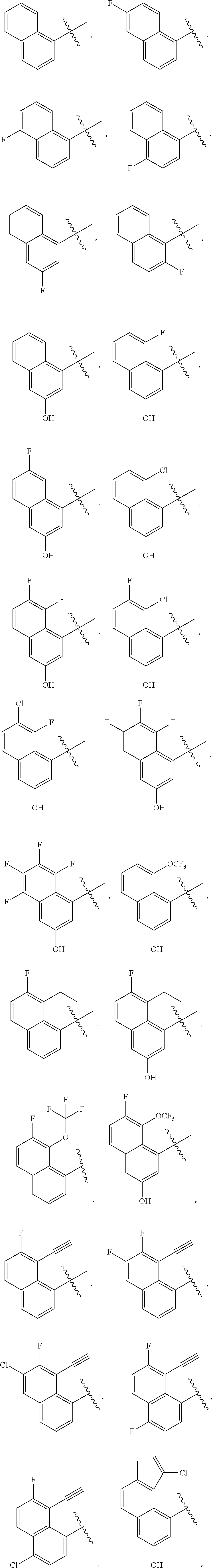

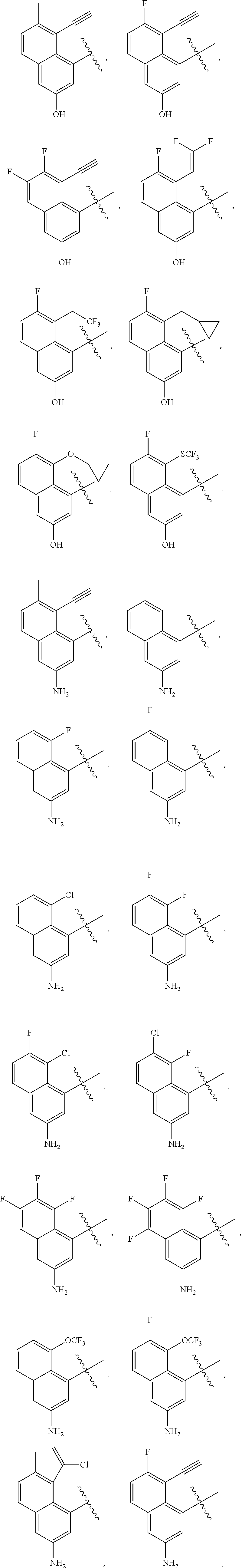

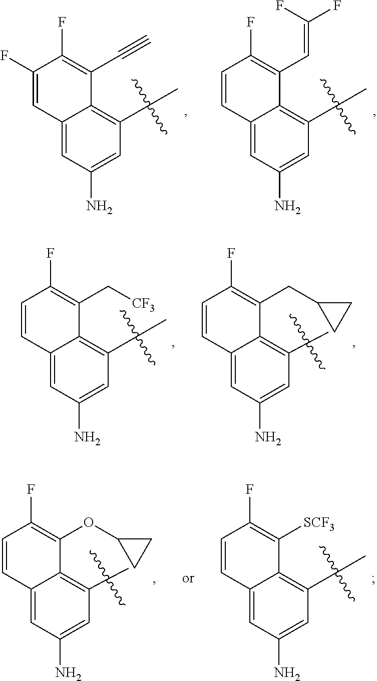

### and R^{D} is F.

In some embodiments, the present disclosure provides the compound of Formula (I), (Ia), and/or (Ib), or a pharmaceutically acceptable salt thereof, wherein R^{2f} and R^{2g} are each independently H or C₁-C₃ alkyl.

In some embodiments, the present disclosure provides the compound of Formula (I), (Ia), and/or (Ib), or a pharmaceutically acceptable salt thereof, wherein Z¹ is —CH₂—.

As described herein, the ring designated as

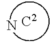

in the compound of Formula (I), (Ia), and/or (Ib), or a pharmaceutically acceptable salt thereof, is also referred to herein interchangeably as ring C2.

In some embodiments, the present disclosure provides the compound of Formula (I), (Ia), and/or (Ib), or a pharmaceutically acceptable salt thereof, wherein ring C2 is 6- to 14-membered heterocyclyl having at least one ring nitrogen, wherein the heterocyclyl has 1, 2, or 3 additional heteroatoms selected from N, O, and S; each R^{C2} is independently C₁-C₆ alkyl or —OH; and n is 0 or 1.

In some embodiments, the present disclosure provides the compound of Formula (I), (Ia), and/or (Ib), or a pharmaceutically acceptable salt thereof, wherein ring C2 is 6- to 14-membered heterocyclyl having at least one ring nitrogen, wherein the heterocyclyl has 1 or 2 additional heteroatoms selected from N and O; each R^{C2} is independently C₁-C₆ alkyl or —OH; and n is 0 or 1.

In some embodiments, the present disclosure provides the compound of Formula (I), (Ia), and/or (Ib), or a pharmaceutically acceptable salt thereof, wherein
ring C2 is

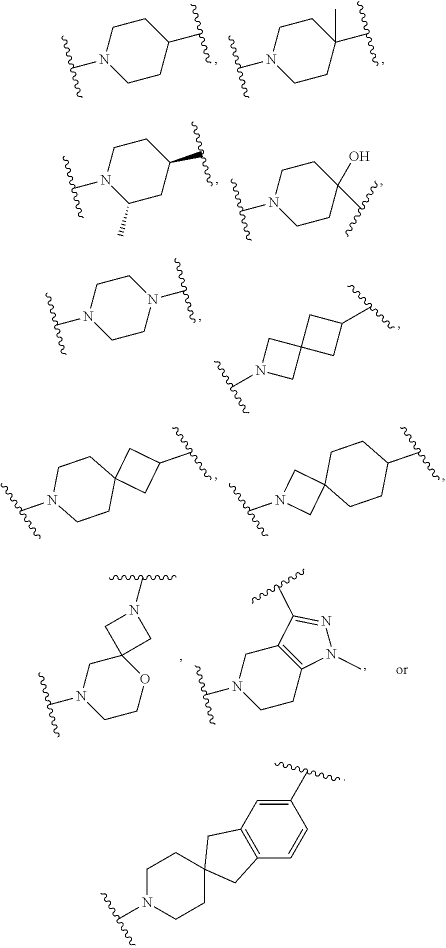

In some embodiments, the present disclosure provides the compound of Formula (I), (Ia), and/or (Ib), or a pharmaceutically acceptable salt thereof, wherein ring C2 is 6- to 14-membered heterocyclyl having at least one ring nitrogen, wherein the heterocyclyl has 1, 2, or 3 additional heteroatoms selected from N, O, and S; each R^{C2} is independently C₁-C₆ alkyl, halo, or —OH; and n is 0, 1, or 2.

In some embodiments, the present disclosure provides the compound of Formula (I), (Ia), and/or (Ib), or a pharmaceutically acceptable salt thereof, wherein ring C2 is

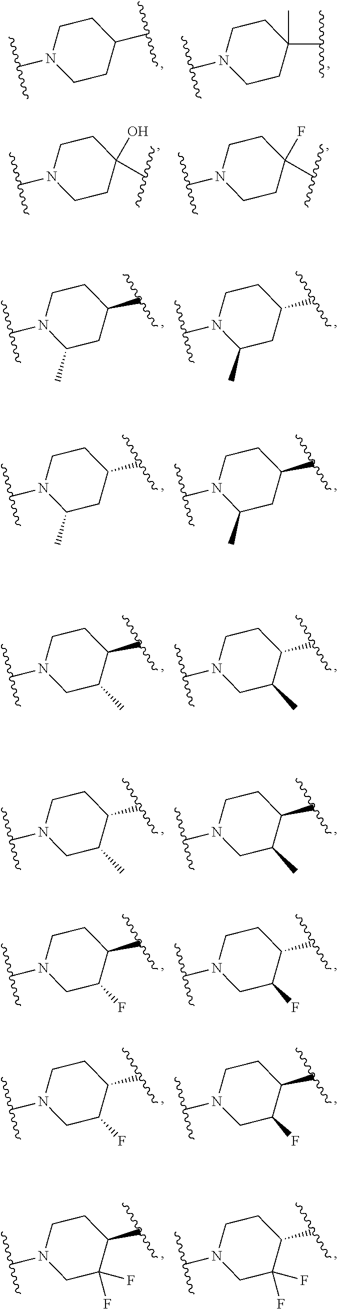

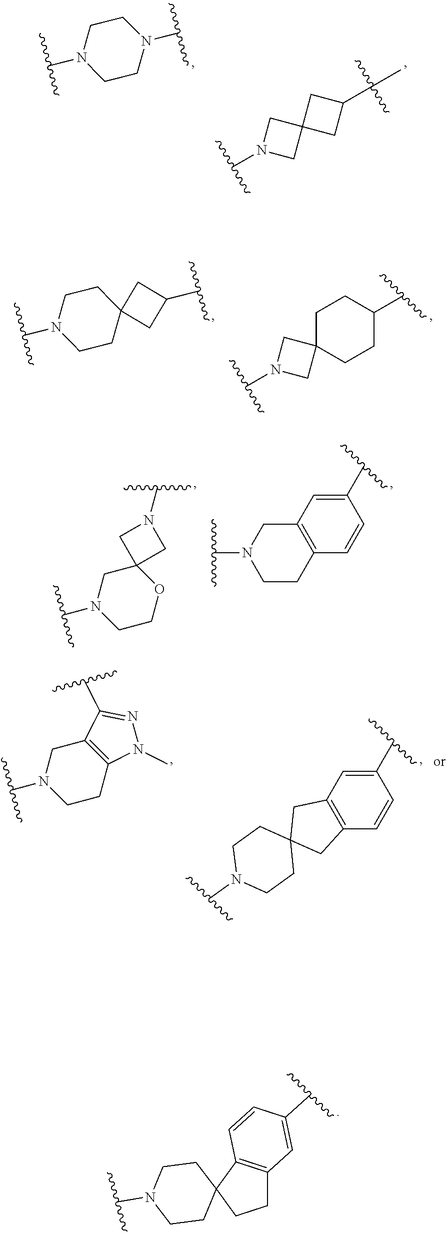

As understood in the art,
ring C2 is attached through a ring nitrogen directly to the carbonyl (—C(═O)—) group of the carbamate attached via an oxygen to Z¹.

In some embodiments, the present disclosure provides the compound of Formula (I), (Ia), and/or (Ib), or a pharmaceutically acceptable salt thereof, wherein Z² is C₁-C₃ alkyl, C₂-C₃ alkynyl, —O—C₁-C₃ alkyl, —O—C₂-C₃ alkynyl, —O—, or a bond. In some embodiments, the present disclosure provides the compound of Formula (I), (Ia), and/or (Ib), or a pharmaceutically acceptable salt thereof, wherein Z² is CH₂, —CH₂CH₂—, —C≡C—, —OCH₂—, —CH₂O—, —OCH₂—C≡C—, —O—, or a bond.

In some embodiments, the present disclosure provides the compound of Formula (I), (Ia), and/or (Ib), or a pharmaceutically acceptable salt thereof, wherein Z² is C₁-C₃ alkyl, C₁-C₃ alkyl substituted with 1 or 2 halo, C₂-C₃ alkynyl, —O—C₁-C₃ alkyl, —O—C₂-C₃ alkynyl, —O—, or a bond. In some embodiments, the present disclosure provides the compound of Formula (I), (Ia), and/or (Ib), or a pharmaceutically acceptable salt thereof, wherein Z² is CH₂, CH(CH₃), C(CH₃)₂, —CH₂CH₂—, —CH₂CH₂CH₂—, CHF, CF₂,

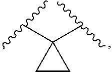 [SMILES: *C1(*)CC1]

—C≡C—, —OCH₂—, —CH₂O—, —OCH₂—C≡C—, —O—, or a bond.

As described herein, the ring designated as

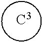

in the compound of Formula (I), (Ia), and/or (Ib), or a pharmaceutically acceptable salt thereof, is also referred to herein interchangeably as ring C3.

In some embodiments, the present disclosure provides the compound of Formula (I), (Ia), and/or (Ib), or a pharmaceutically acceptable salt thereof, wherein
ring C3 is 4- to 16-membered heterocyclyl or absent, wherein the heterocyclyl has 1, 2, 3, or 4 heteroatoms selected from N, O, and S; each R^{C3} is independently C₁-C₆ alkyl; and p is 0 or 1.

In some embodiments, the present disclosure provides the compound of Formula (I), (Ia), and/or (Ib), or a pharmaceutically acceptable salt thereof, wherein
ring C3 is 4- to 16-membered heterocyclyl or absent, wherein the heterocyclyl has 1 or 2 heteroatoms that is N; each R^{C3} is independently C₁-C₆ alkyl; and p is 0 or 1.

In some embodiments, the present disclosure provides the compound of Formula (I), (Ia), and/or (Ib), or a pharmaceutically acceptable salt thereof, wherein
ring C3 is 6- to 9-membered heterocyclyl or absent, wherein the heterocyclyl has 1 or 2 heteroatoms that is N; each R^{C3} is independently C₁-C₆ alkyl; and p is 0 or 1.

In some embodiments, the present disclosure provides the compound of Formula (I), (Ia), and/or (Ib), or a pharmaceutically acceptable salt thereof, wherein
ring C3 is:

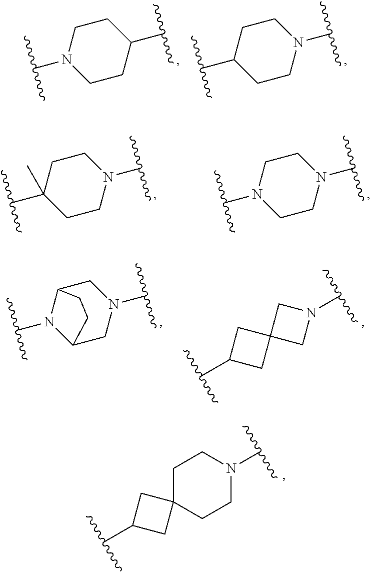

absent.

In some embodiments, the present disclosure provides the compound of Formula (I), (Ia), and/or (Ib), or a pharmaceutically acceptable salt thereof, wherein ring C3 is C₃-C₈ cycloalkyl, 4- to 16-membered heterocyclyl, 5- to 10-membered heteroaryl, or absent, wherein the heterocyclyl and heteroaryl have 1, 2, 3, or 4 heteroatoms selected from N, O, and S; each R^{C3} is independently C₁-C₆ alkyl or halo; and p is 0, 1, or 2. In some embodiments, the present disclosure provides the compound of Formula (I), (Ia), and/or (Ib), or a pharmaceutically acceptable salt thereof, wherein ring C3 is:

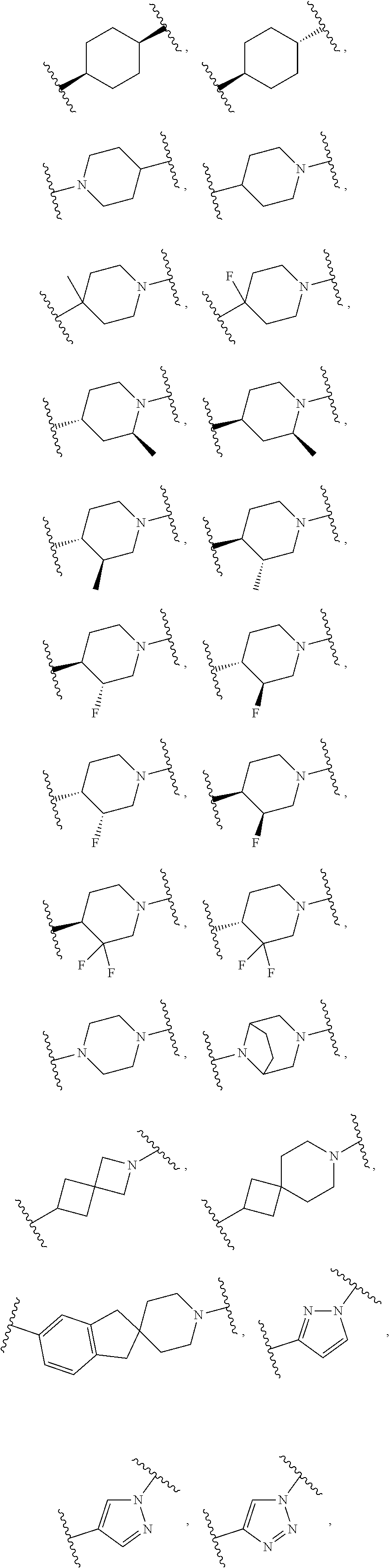

or absent.

In some embodiments, the present disclosure provides the compound of Formula (I), (Ia), and/or (Ib), or a pharmaceutically acceptable salt thereof, wherein Z⁵ is C₁-C₃ alkyl, C₂-C₃ alkynyl, —O—C₁-C₃ alkyl, —O—C₂-C₃ alkynyl, —O—, or a bond. In some embodiments, the present disclosure provides the compound of Formula (I), (Ia), and/or (Ib), or a pharmaceutically acceptable salt thereof, wherein Z⁵ is CH₂, —CH₂CH₂—, —C≡C—, —OCH₂—, —CH₂O—, —OCH₂—C≡C—, —O—, or a bond. In some embodiments, the present disclosure provides the compound of Formula (I), (Ia), and/or (Ib), or a pharmaceutically acceptable salt thereof, wherein Z⁵ is a bond. In some embodiments, the present disclosure provides the compound of Formula (I), (Ia), and/or (Ib), or a pharmaceutically acceptable salt thereof, wherein Z⁵ is CH₂.

As described herein, the ring designated as

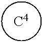

in the compound of Formula (I), (Ia), and/or (Ib), or a pharmaceutically acceptable salt thereof, is also referred to herein interchangeably as ring C4.

In some embodiments, the present disclosure provides the compound of Formula (I), (Ia), and/or (Ib), or a pharmaceutically acceptable salt thereof, wherein ring C4 is 5 to 14-membered heterocyclyl, C₆-C₁₀ aryl or 5- to 10-membered heteroaryl, wherein the heterocyclyl and the heteroaryl each has 1, 2, 3, or 4 heteroatoms selected from N, O, and S; each R^{C4} is independently C₁-C₆ alkyl, halo, or oxo; and q is 0, 1 or 2.

In some embodiments, the present disclosure provides the compound of Formula (I), (Ia), and/or (Ib), or a pharmaceutically acceptable salt thereof, wherein
ring C4 is 5 to 14-membered heterocyclyl, C₆-C₁₀ aryl or 5- to 10-membered heteroaryl, wherein the heterocyclyl and the heteroaryl each has 1 or 2 heteroatoms that is N; each R^{C4} is independently C₁-C₆ alkyl, halo, or oxo; and q is 0, 1 or 2.

In some embodiments, the present disclosure provides the compound of Formula (I), (Ia), and/or (Ib), or a pharmaceutically acceptable salt thereof, wherein
ring C4 is 9-membered heterocyclyl, phenyl, or 6- to 9-membered heteroaryl, wherein the heterocyclyl and the heteroaryl each has 1 or 2 heteroatoms that is N; each R^{C4} is independently C₁-C₆ alkyl, halo, or oxo; and q is 0, 1 or 2.

In some embodiments, the present disclosure provides the compound of Formula (I), (Ia), and/or (Ib), or a pharmaceutically acceptable salt thereof, wherein
ring C4 is

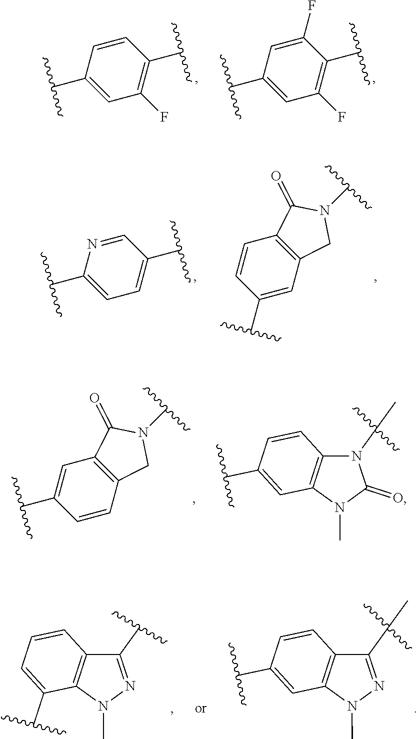

In some embodiments, the present disclosure provides the compound of Formula (I), (Ia), and/or (Ib), or a pharmaceutically acceptable salt thereof, wherein
ring C4 is

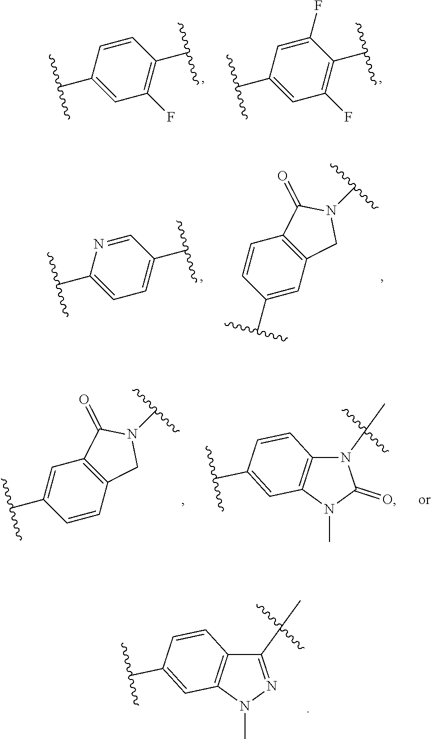

In some embodiments, the present disclosure provides the compound of Formula (I), (Ia), and/or (Ib), or a pharmaceutically acceptable salt thereof, wherein ring C4 is 5 to 14-membered heterocyclyl, C₆-C₁₀ aryl or 5- to 10-membered heteroaryl, wherein the heterocyclyl and the heteroaryl each has 1, 2, 3, or 4 heteroatoms selected from N, O, and S; each R^{C4} is independently C₁-C₆ alkyl, halo, oxo, or C₃-C₆ cycloalkyl; and q is 0, 1, 2, or 3. In some embodiments, the present disclosure provides the compound of Formula (I), (Ia), and/or (Ib), or a pharmaceutically acceptable salt thereof, wherein ring C4 is

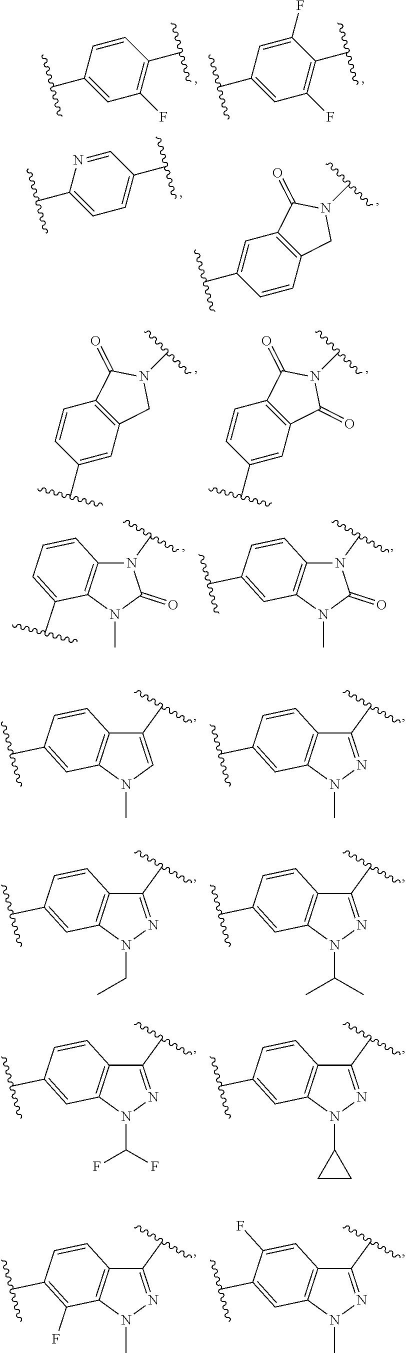

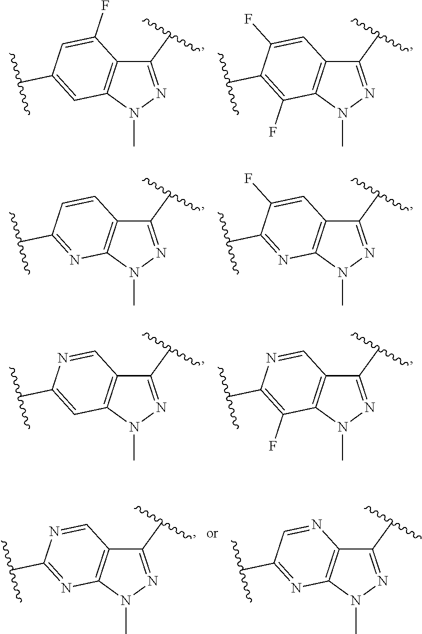

In some embodiments, the present disclosure provides the compound of Formula (I), (Ia), and/or (Ib), or a pharmaceutically acceptable salt thereof, wherein Z³ is a bond.

In some embodiments, the present disclosure provides the compound of Formula (I), (Ia), and/or (Ib), or a pharmaceutically acceptable salt thereof, wherein Z⁴ is —CH—.

In some embodiments, the present disclosure provides the compound of Formula (I), (Ia), and/or (Ib), or a pharmaceutically acceptable salt thereof, wherein Z⁴ is N.

In some embodiments, the present disclosure provides the compound of Formula (I), (Ia), and/or (Ib), or a pharmaceutically acceptable salt thereof, wherein Z¹ is —CH₂—;
ring C2 is 6- to 14-membered heterocyclyl having at least one ring nitrogen, wherein the heterocyclyl has 1, 2, or 3 additional heteroatoms selected from N, O, and S; each R^{C2} is independently C₁-C₆ alkyl or —OH; n is 0 or 1; Z² is C₁-C₃ alkyl, C₂-C₃ alkynyl, —O—C₁-C₃ alkyl, —O—C₂-C₃ alkynyl, —O—, or a bond;
ring C3 is 4- to 16-membered heterocyclyl or absent, wherein the heterocyclyl has 1 or 2 heteroatoms that is N; each R^{C3} is independently C₁-C₆ alkyl; p is 0 or 1; Z⁵ is a bond;
ring C4 is 5 to 14-membered heterocyclyl, C₆-C₁₀ aryl or 5- to 10-membered heteroaryl, wherein the heterocyclyl and the heteroaryl each has 1 or 2 heteroatoms that is N; each R^{C4} is independently C₁-C₆ alkyl, halo, or oxo; q is 0, 1 or 2; Z³ is a bond; and Z⁴ is —CH— or N.

In some embodiments, the present disclosure provides the compound of Formula (I), (Ia), and/or (Ib), or a pharmaceutically acceptable salt thereof, wherein
X is N;
L¹ is CR^{1a}R^{1b} or —C(R^{1e})═;
R^{1a} and R^{1b} are each independently H, C₁-C₃ alkyl, or halo;
R^{1e} is H or C₁-C₃ alkyl;
L² is CHR^{2b} or ═C(R^{2e})—;
R^{2b} is H, C₁-C₃ alkyl, or halo;
R^{2e} is H or C₁-C₃ alkyl;
L³ is a bond;
R^{A} is phenyl or naphthyl, wherein R^{A} is substituted with 0, 1, 2, or 3 R^{A2};
each R^{A2} is independently —OH, C₁-C₁₀ alkyl, C₂-C₆ alkynyl, C₁-C₁₀ alkoxy, halo, —N(R^{A2a})(R^{A2b}), wherein each alkyl, alkynyl, and alkoxy is substituted with 0, 1, 2, or 3 R^{A3};
each R^{A2a} and R^{A2b} is independently H or C₁-C₁₀ alkyl;
each R^{A3} is independently halo;
alternatively, two R^{A2} can combine to form a C₃-C₁₀ cycloalkyl or C₆-C₁₀ aryl on two adjacent atoms on R^{A}, wherein each cycloalkyl and aryl is substituted with 0, 1, 2, or 3 R^{A5};
each R^{A5} is independently H, C₁-C₁₀ alkyl, C₂-C₁₀ alkynyl, halo, C₁-C₆ haloalkyl, or C₃-C₈ cycloalkyl;
R^{D} is F;
Z¹ is —CH₂—;
ring C2 is 6- to 14-membered heterocyclyl having at least one ring nitrogen, wherein the heterocyclyl has 1, 2, or 3 additional heteroatoms selected from N, O, and S;
each R^{C2} is independently C₁-C₆ alkyl, halo, or —OH;
n is 0, 1, or 2;
Z² is C₁-C₃ alkyl, C₁-C₃ alkyl substituted with 1 or 2 halo, C₂-C₃ alkynyl, —O—C₁-C₃ alkyl, —O—C₂-C₃ alkynyl, —O—, or a bond;
ring C3 is C₃-C₈ cycloalkyl, 4- to 16-membered heterocyclyl, 5- to 10-membered heteroaryl, or absent, wherein the heterocyclyl and heteroaryl have 1, 2, 3, or 4 heteroatoms selected from N, O, and S;
each R^{C3} is independently C₁-C₆ alkyl or halo;
p is 0, 1, or 2;
Z⁵ is CH₂ or a bond;
ring C4 is 5 to 14-membered heterocyclyl, C₆-C₁₀ aryl or 5- to 10-membered heteroaryl, wherein the heterocyclyl and the heteroaryl each has 1, 2, 3, or 4 heteroatoms selected from N, O, and S;
each R^{C4} is independently C₁-C₆ alkyl, halo, oxo, or C₃-C₆ cycloalkyl; and
q is 0, 1, 2, or 3;
Z³ is a bond; and
Z⁴ is —CH— or N.

In some embodiments, the present disclosure provides the compound of Formula (I), (Ia), and/or (Ib), or a pharmaceutically acceptable salt thereof, wherein

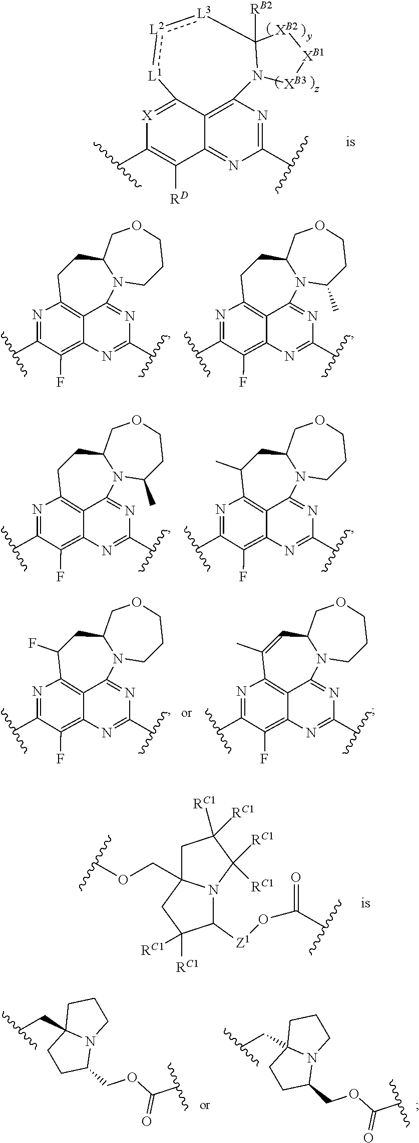

R^{A} is:

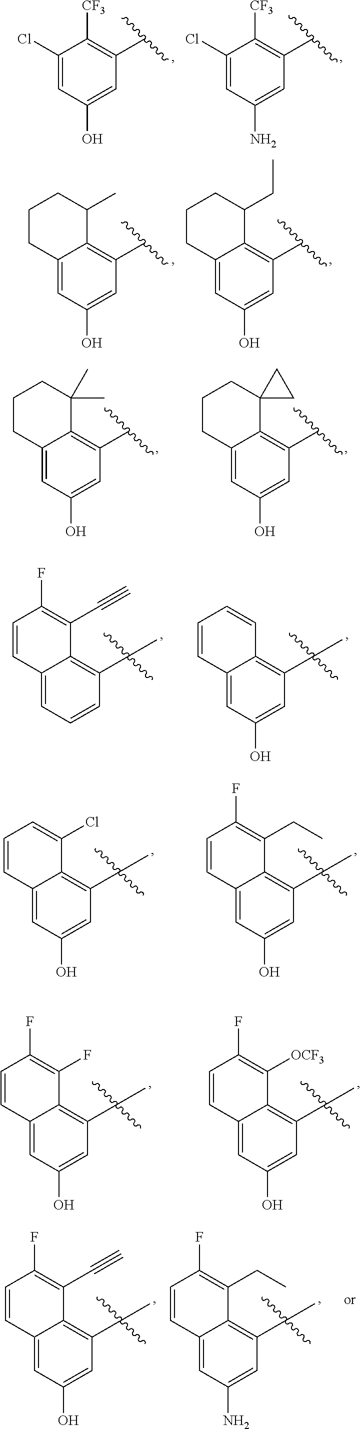

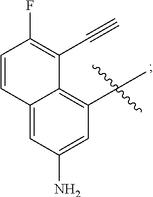 [SMILES: *c1cc(N)cc2ccc(F)c(C#C)c12]

ring C2 is

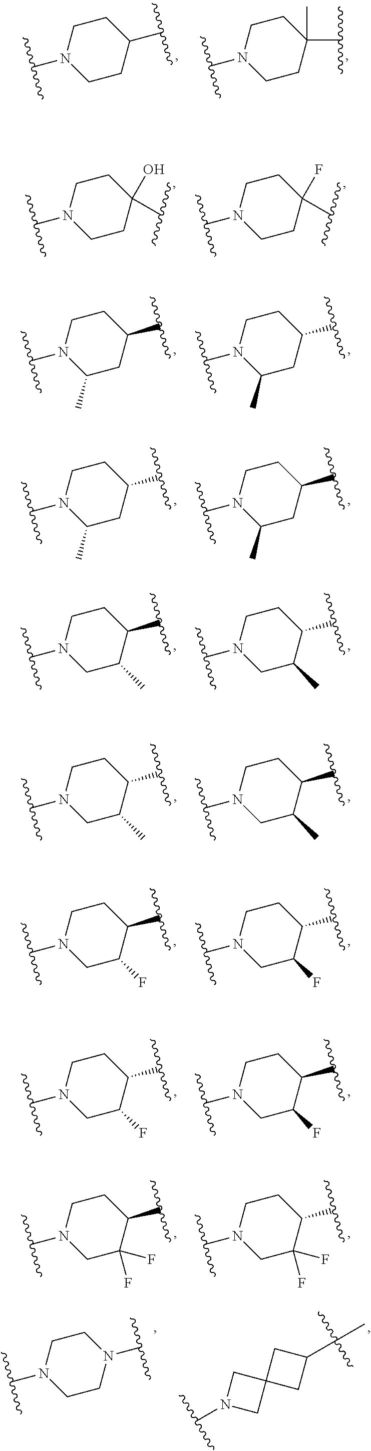

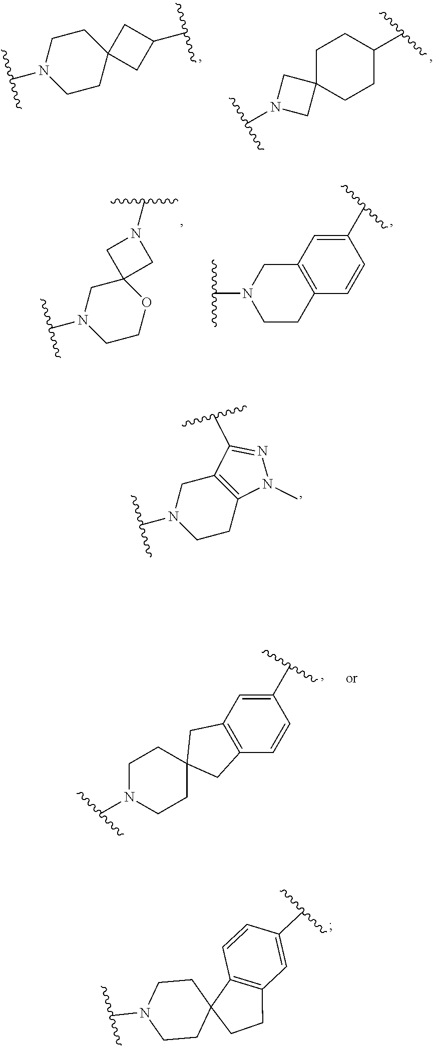

Z² is CH₂, CH(CH₃), C(CH₃)₂, —CH₂CH₂—, —CH₂CH₂CH₂—, CHF, CF₂,

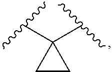 [SMILES: *C1(*)CC1]

—C≡C—, —OCH₂—, —CH₂O—, —OCH₂—C≡C—, —O—, or a bond;
ring C3 is absent or is:

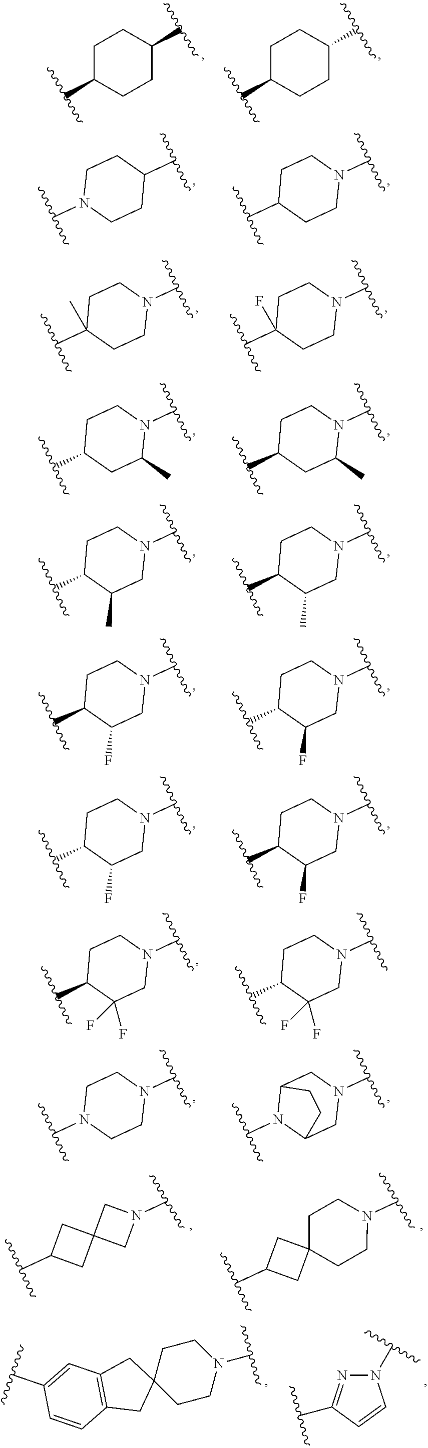

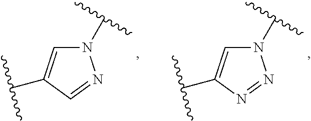 [SMILES: *c1cnn(*)c1 ; *c1cn(*)nn1]

or absent;
Z⁵ is absent or CH₂;
ring C4 is

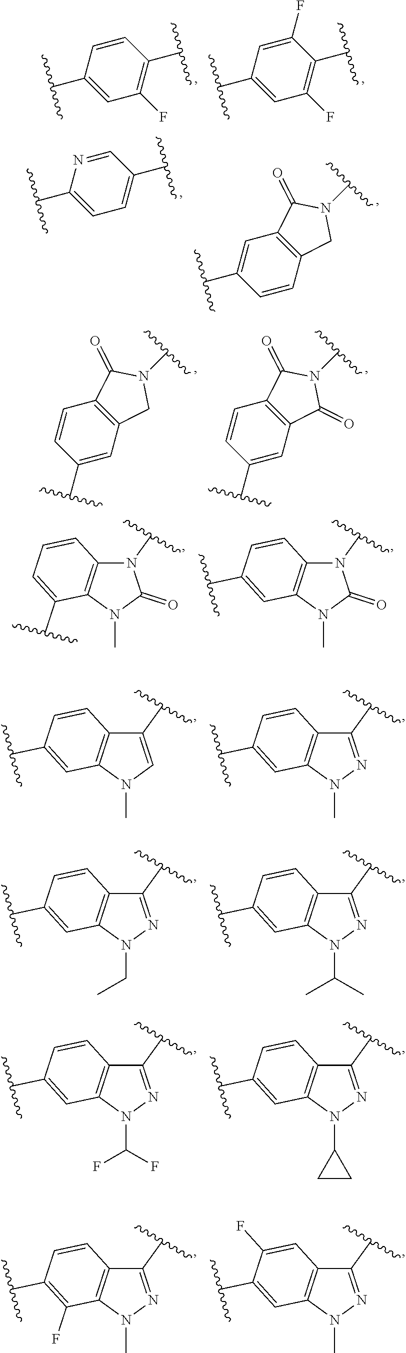

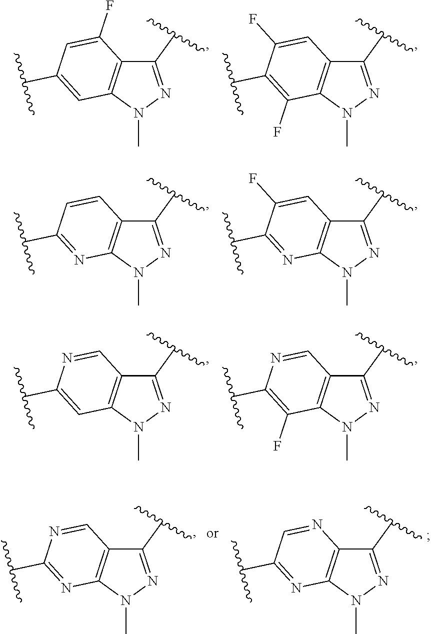

and
Z⁴ is —CH— or N.

In some embodiments, the present disclosure provides the compound of Formula (I), (Ia), and/or (Ib), or a pharmaceutically acceptable salt thereof, wherein the moiety

[chemical structure]

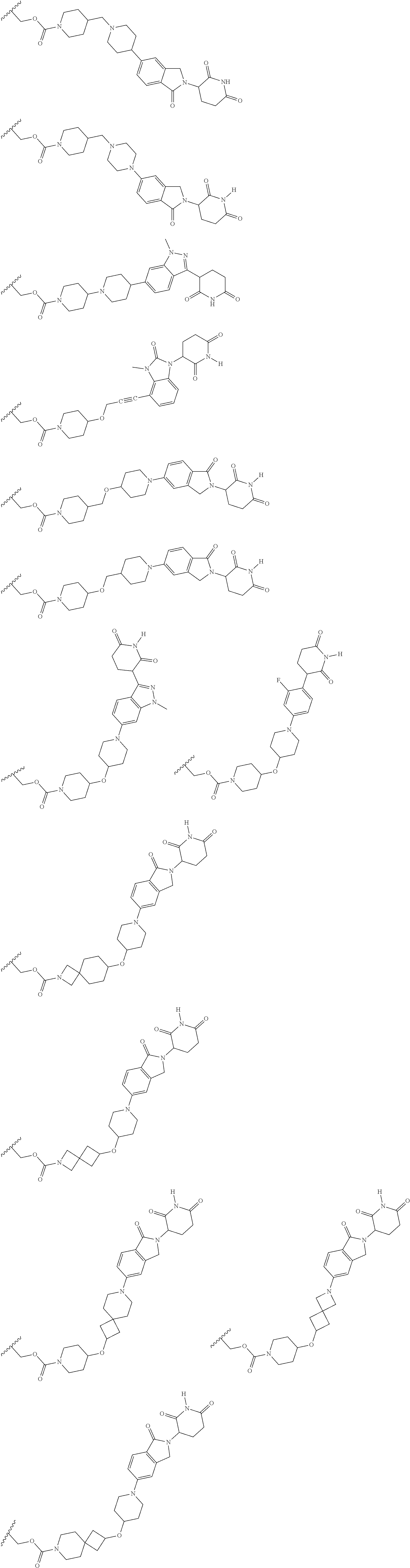

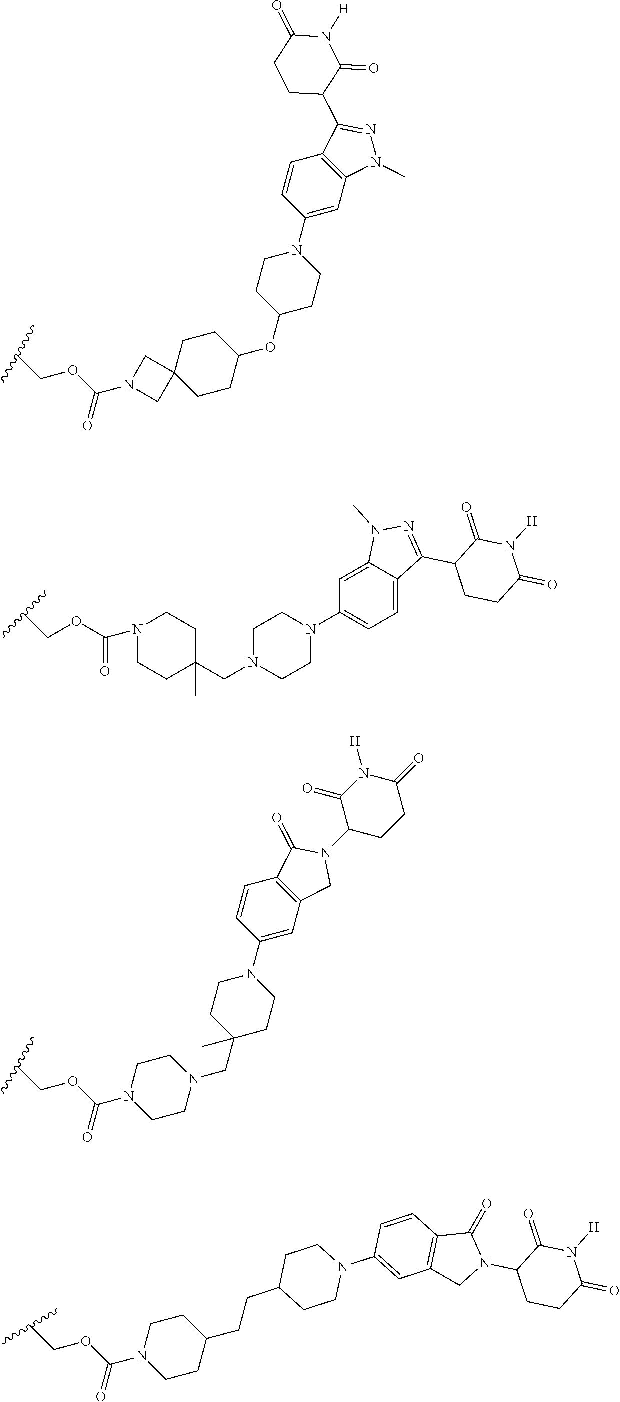 [SMILES: *COC(=O)N1CC2(CCC(OC3CCN(c4ccc5c(C6CCC(=O)NC6=O)nn(C)c5c4)CC3)CC2)C1 ; *COC(=O)N1CCC(C)(CN2CCN(c3ccc4c(C5CCC(=O)NC5=O)nn(C)c4c3)CC2)CC1 ; *COC(=O)N1CCN(CC2(C)CCN(c3ccc4c(c3)CN(C3CCC(=O)NC3=O)C4=O)CC2)CC1 ; *COC(=O)N1CCC(CCC2CCN(c3ccc4c(c3)CN(C3CCC(=O)NC3=O)C4=O)CC2)CC1]

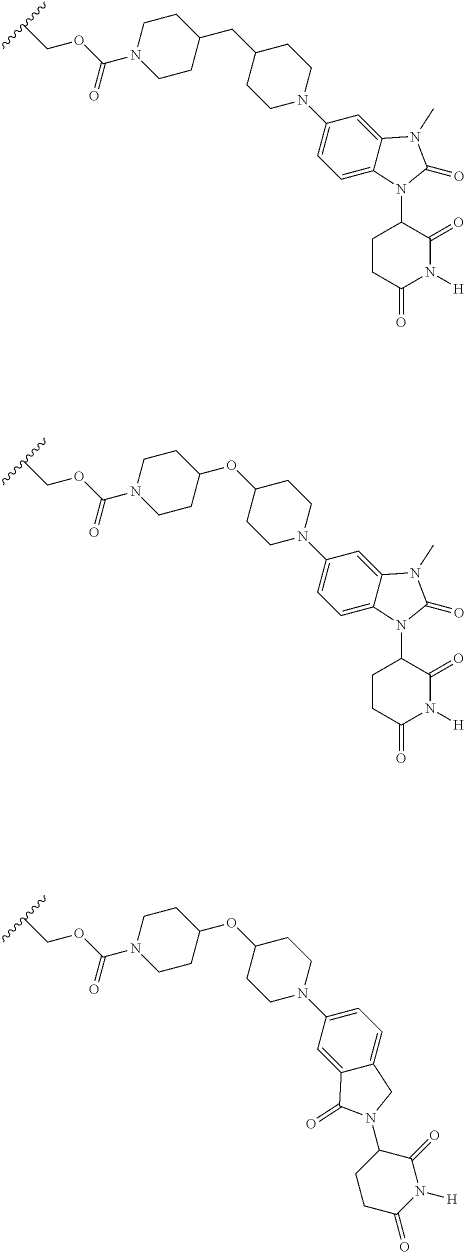 [SMILES: *COC(=O)N1CCC(CC2CCN(c3ccc4c(c3)n(C)c(=O)n4C3CCC(=O)NC3=O)CC2)CC1 ; *COC(=O)N1CCC(OC2CCN(c3ccc4c(c3)n(C)c(=O)n4C3CCC(=O)NC3=O)CC2)CC1 ; *COC(=O)N1CCC(OC2CCN(c3ccc4c(c3)C(=O)N(C3CCC(=O)NC3=O)C4)CC2)CC1]

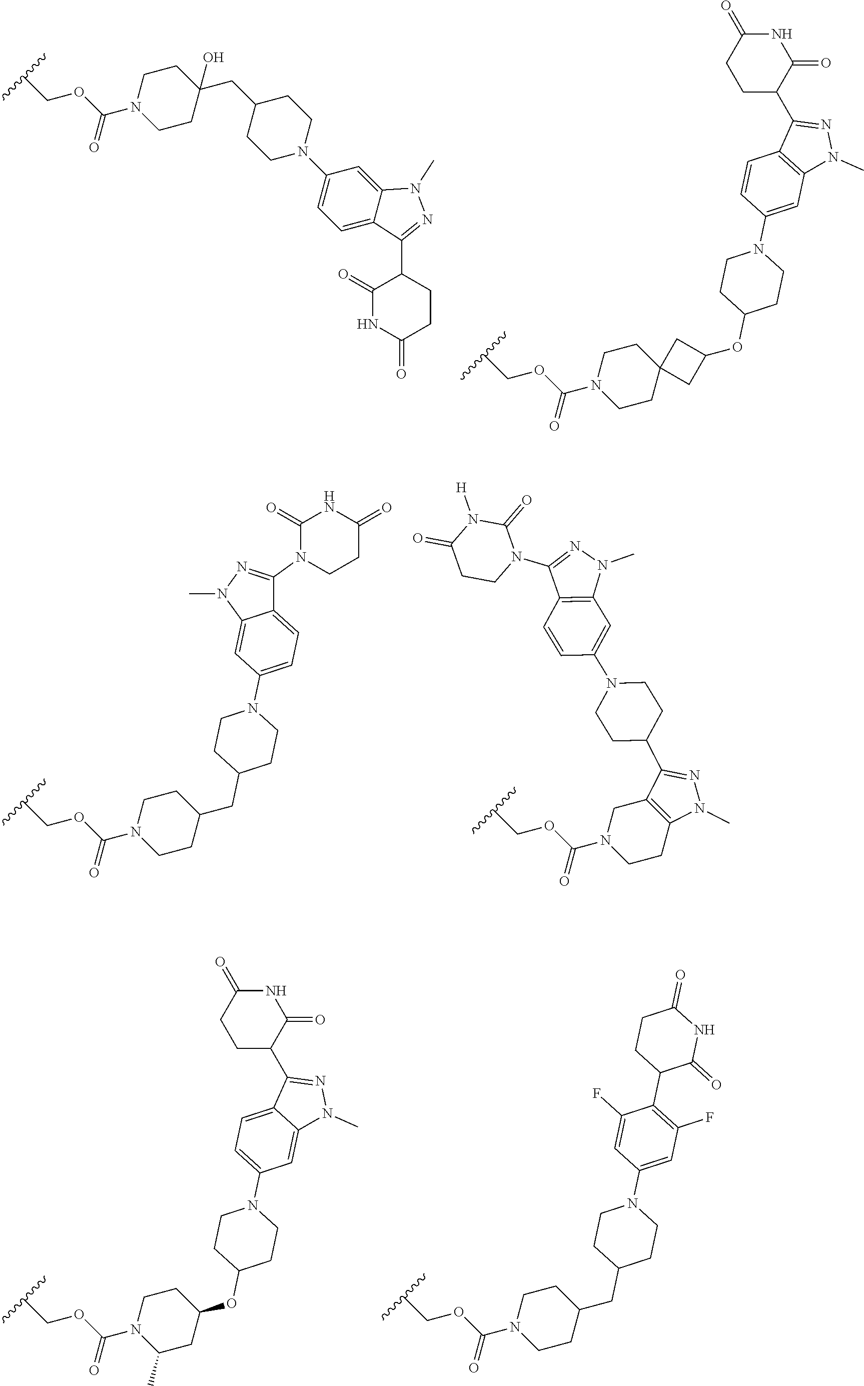

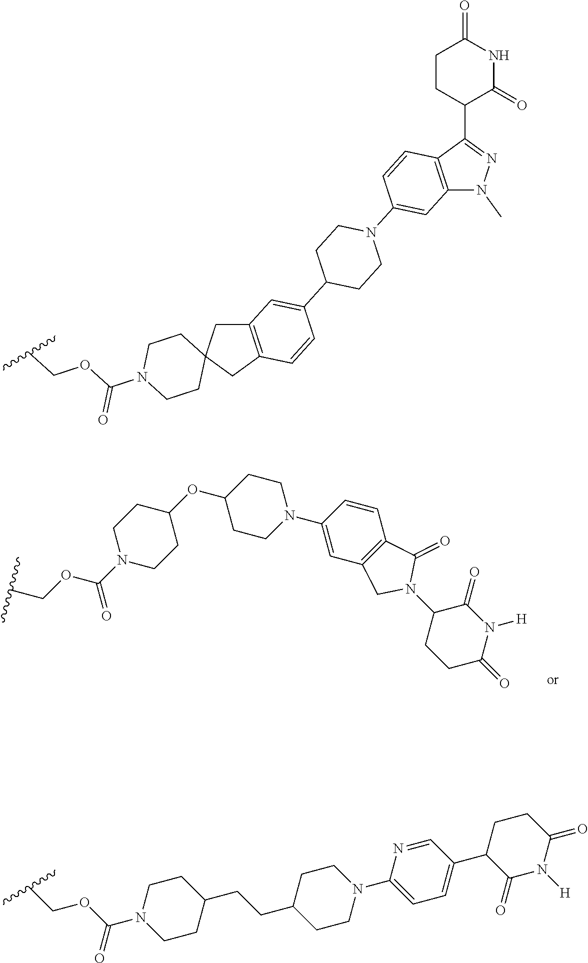 [SMILES: *COC(=O)N1CCC2(CC1)Cc1ccc(C3CCN(c4ccc5c(C6CCC(=O)NC6=O)nn(C)c5c4)CC3)cc1C2 ; *COC(=O)N1CCC(OC2CCN(c3ccc4c(c3)CN(C3CCC(=O)NC3=O)C4=O)CC2)CC1 ; *COC(=O)N1CCC(CCC2CCN(c3ccc(C4CCC(=O)NC4=O)cn3)CC2)CC1]

or wherein the moiety is:

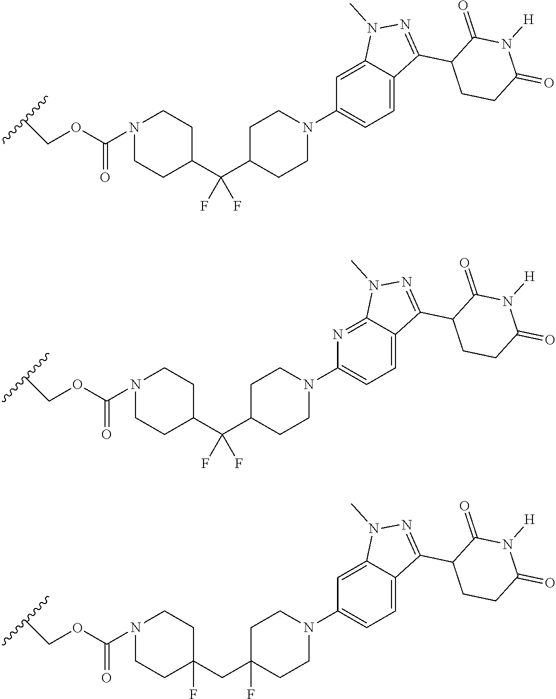 [SMILES: *COC(=O)N1CCC(C(F)(F)C2CCN(c3ccc4c(C5CCC(=O)NC5=O)nn(C)c4c3)CC2)CC1 ; *COC(=O)N1CCC(C(F)(F)C2CCN(c3ccc4c(C5CCC(=O)NC5=O)nn(C)c4n3)CC2)CC1 ; *COC(=O)N1CCC(F)(CC2(F)CCN(c3ccc4c(C5CCC(=O)NC5=O)nn(C)c4c3)CC2)CC1]

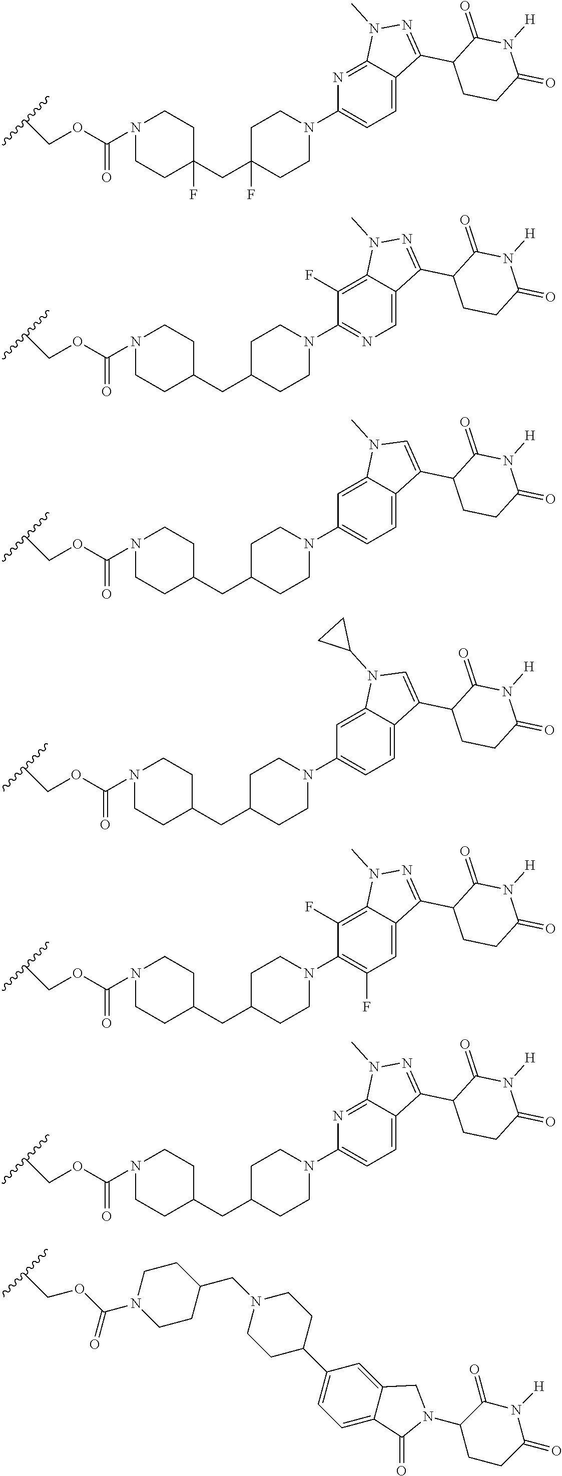

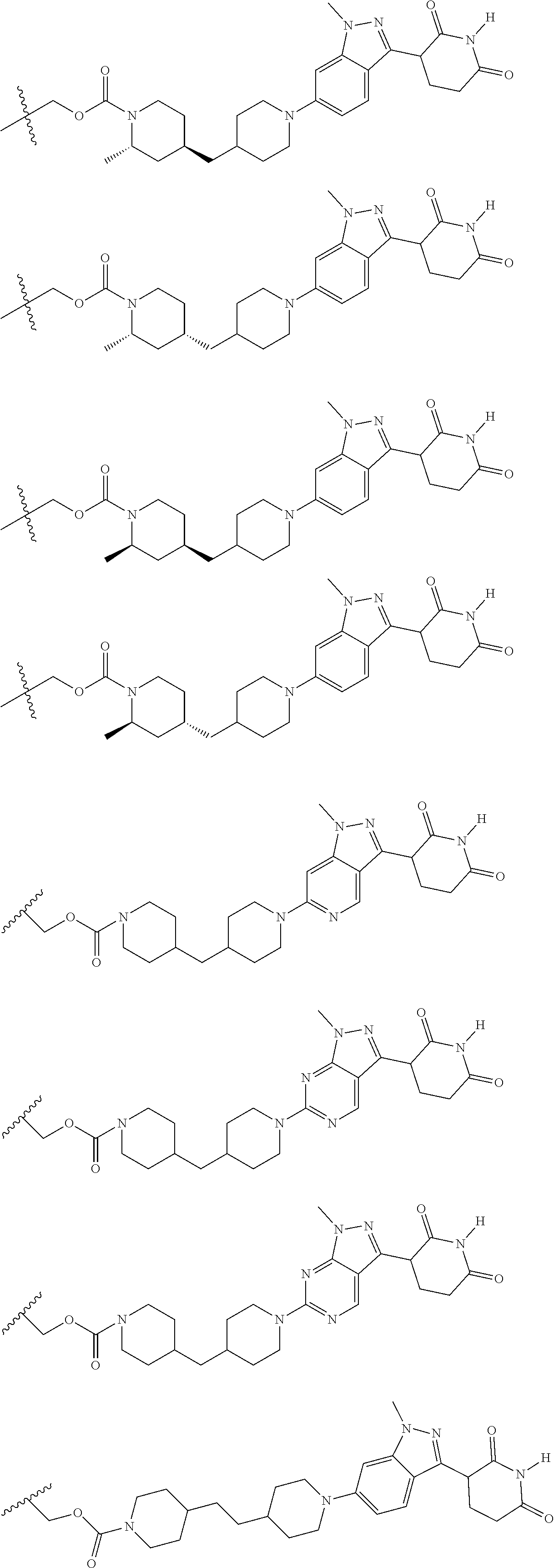

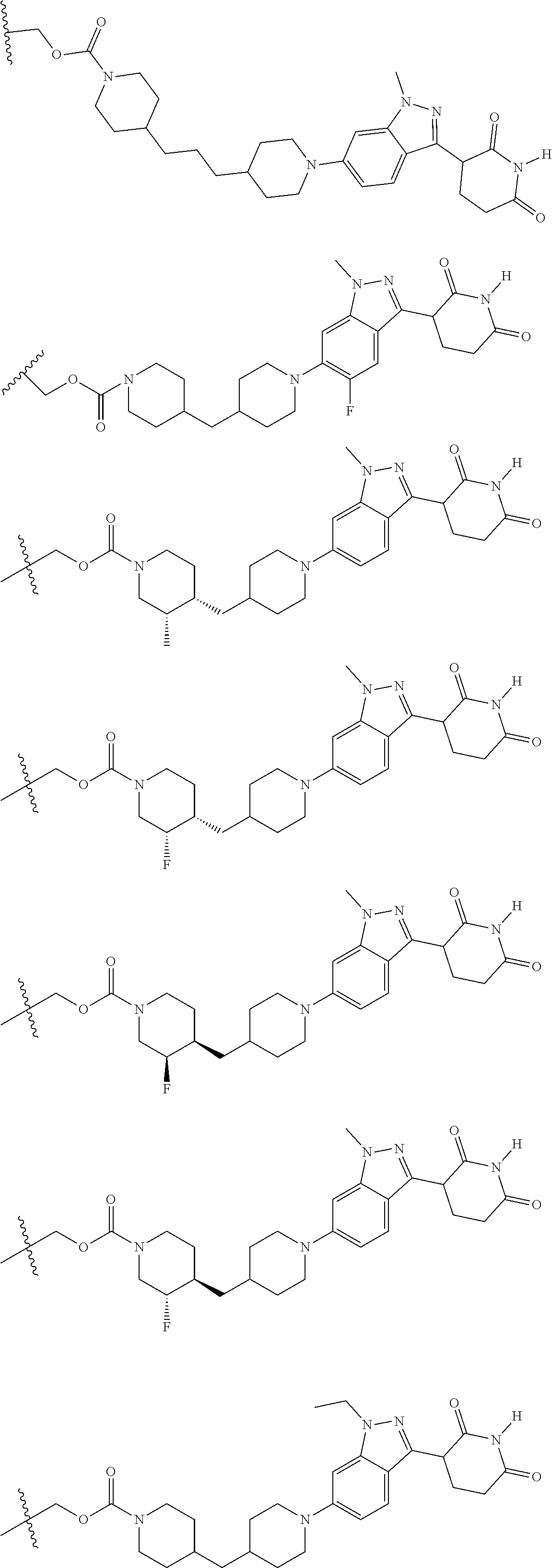

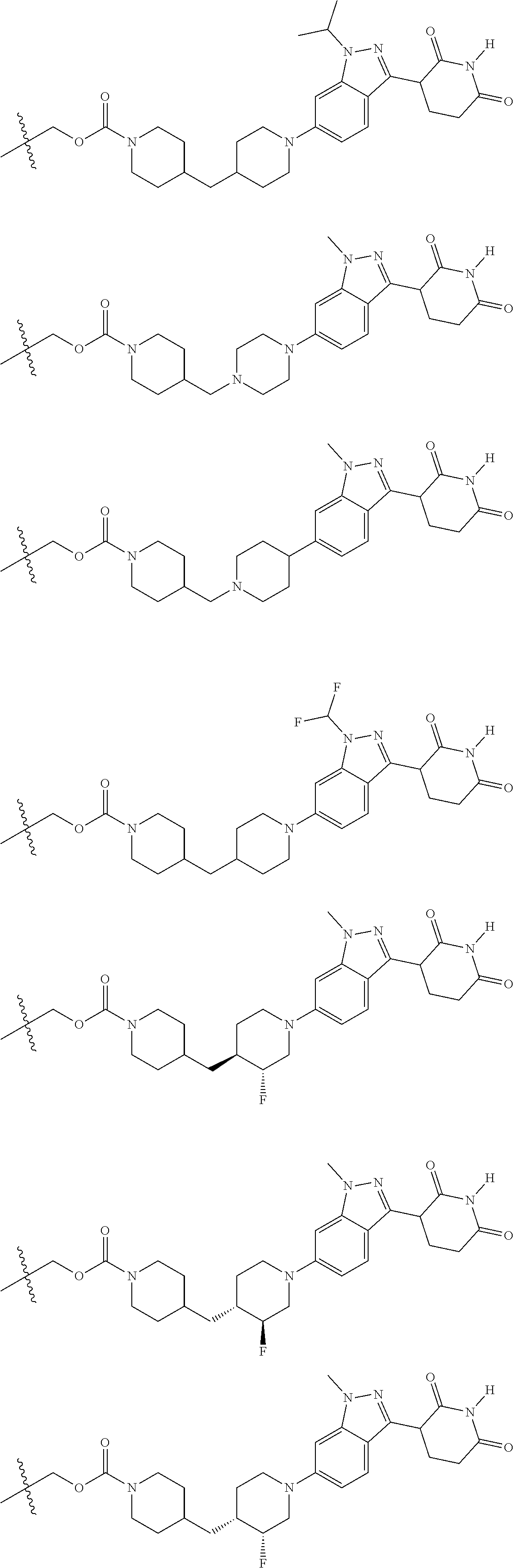

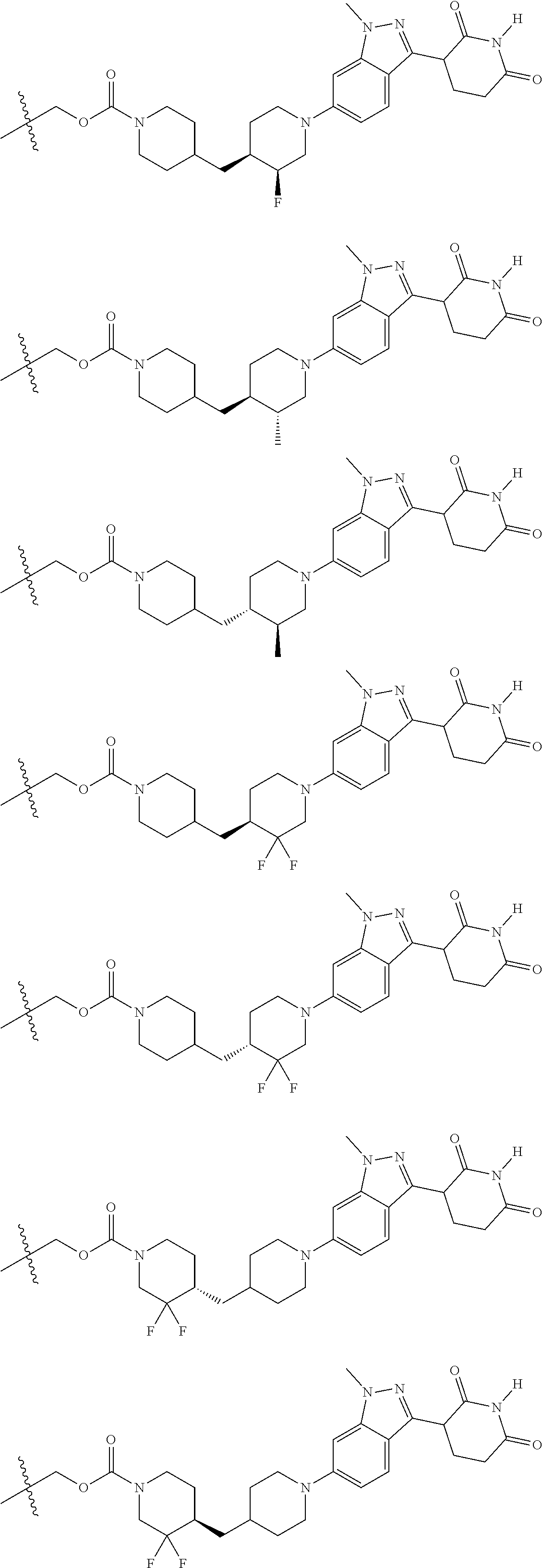

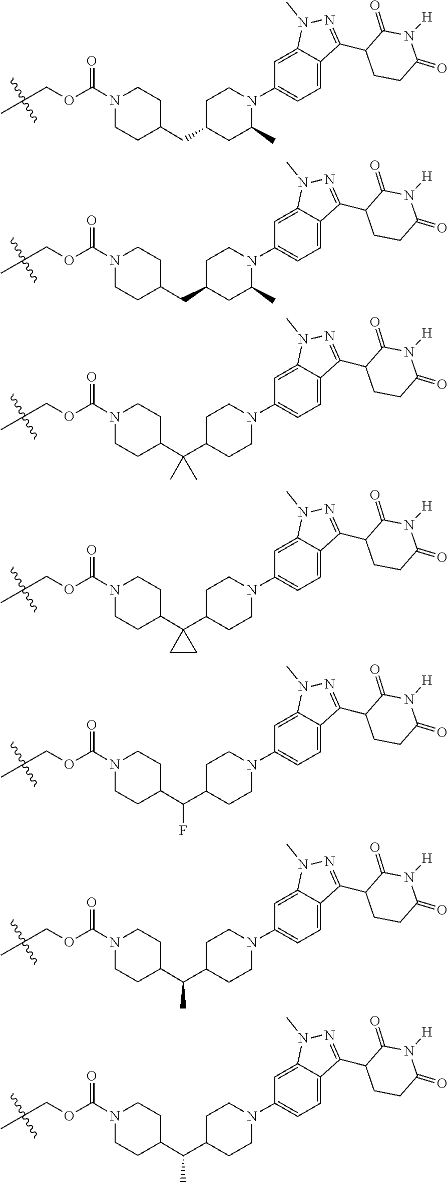

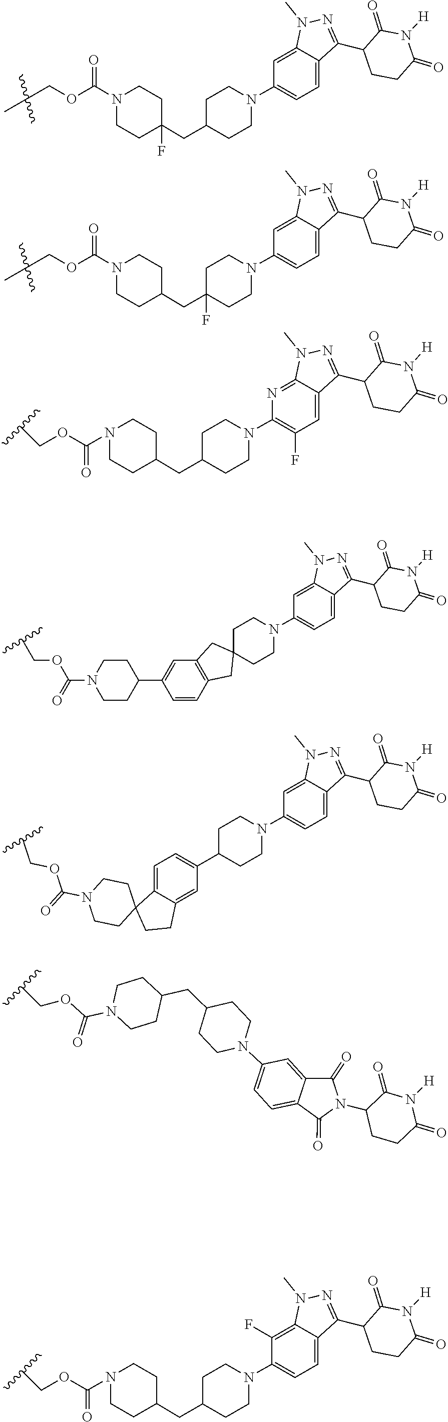

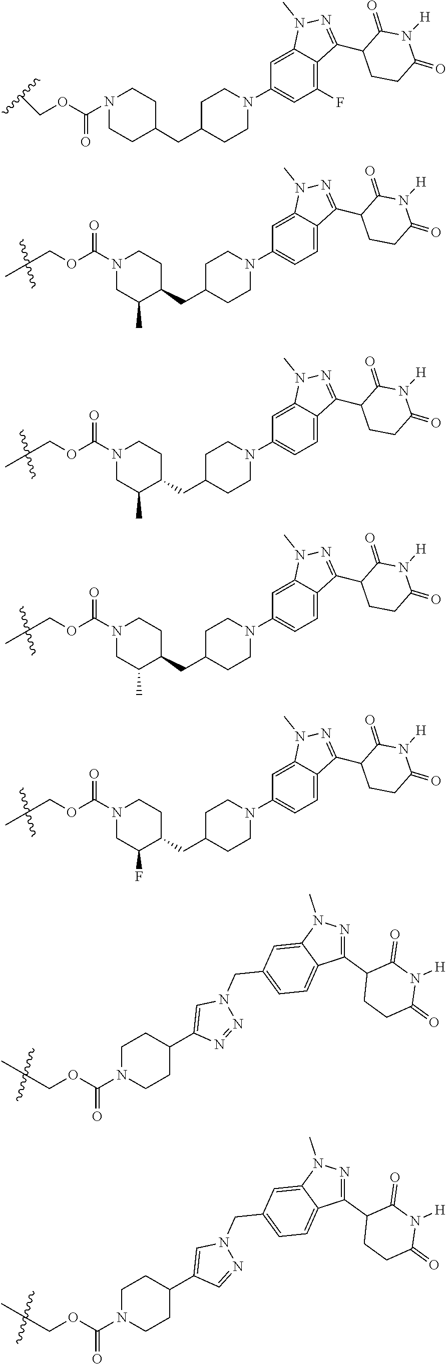

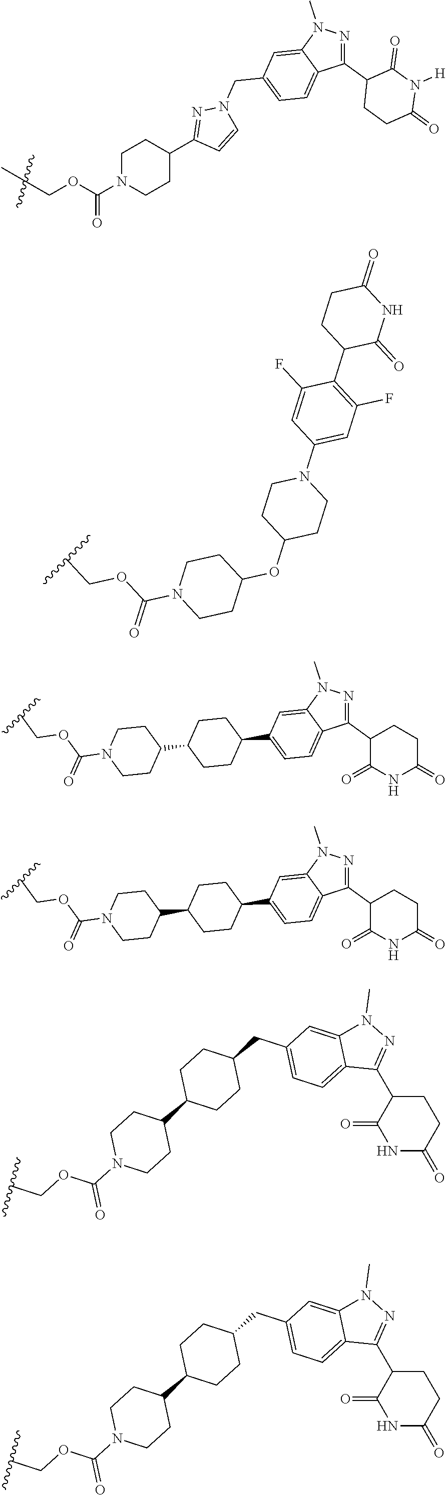 [SMILES: *COC(=O)N1CCC(c2ccn(Cc3ccc4c(C5CCC(=O)NC5=O)nn(C)c4c3)n2)CC1 ; *COC(=O)N1CCC(OC2CCN(c3cc(F)c(C4CCC(=O)NC4=O)c(F)c3)CC2)CC1 ; *COC(=O)N1CCC([C@H]2CC[C@H](c3ccc4c(C5CCC(=O)NC5=O)nn(C)c4c3)CC2)CC1 ; *COC(=O)N1CCC([C@H]2CC[C@@H](c3ccc4c(C5CCC(=O)NC5=O)nn(C)c4c3)CC2)CC1 ; *COC(=O)N1CCC([C@H]2CC[C@@H](Cc3ccc4c(C5CCC(=O)NC5=O)nn(C)c4c3)CC2)CC1 ; *COC(=O)N1CCC([C@H]2CC[C@H](Cc3ccc4c(C5CCC(=O)NC5=O)nn(C)c4c3)CC2)CC1]

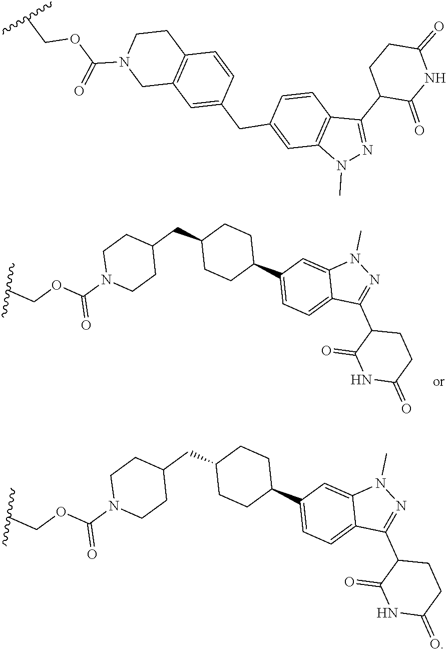

In some embodiments, the present disclosure provides the compound of Formula (I), (Ia), and/or (Ib), or a pharmaceutically acceptable salt thereof, wherein X is N, CH, or CR^{x}; R^{x} is halo; L¹ is O or CR^{1a}R^{1b}; R^{1a} and R^{1b} are each independently H, C₁-C₃ alkyl, or halo; L² is CR^{2a}R^{2b}; R^{2a} and R^{2b} are each independently H, C₁-C₃ alkyl, C₁-C₆ haloalkyl, or C₃-C₆ cycloalkyl; L³ is a bond or CR^{3a}R^{3b}; R^{3a} and R^{3b} are each independently H or C₁-C₃ alkyl; R¹ is H or F; R² is F; R³, R⁴, and R⁵ are each H; R^{A} is naphthyl, wherein R^{A} is substituted with 0, 1, 2, 3, 4, or 5 R^{A2}; each R^{A2} is independently —OH, C₁-C₃ alkyl, C₂-C₆ alkenyl, C₂-C₆ alkynyl, C₁-C₆ alkoxy, C₁-C₆ thioalkyl, halo, C₁-C₆ haloalkyl, —OR^{A2a}, —SR^{A2a}, —N(R^{A2a})(R^{A2b}), or —(C₁-C₆ alkyl)-(C₃-C₈ cycloalkyl), wherein each alkyl, alkenyl, alkynyl, alkoxy, and haloalkyl is substituted with 0, 1, 2, or 3 R^{A3}; each R^{A2a} and R^{A2b} is independently H, C₁-C₆ haloalkyl, or C₃-C₈ cycloalkyl; each R^{A3} is independently halo; R^{D} is F; Z¹ is —CH₂—;
ring C2 is 6- to 14-membered heterocyclyl having at least one ring nitrogen, wherein the heterocyclyl has 1, 2, or 3 additional heteroatoms selected from N, O, and S; each R^{C2} is independently C₁-C₆ alkyl or —OH; n is 0 or 1; Z² is C₁-C₃ alkyl, C₂-C₃ alkynyl, —O—C₁-C₃ alkyl, —O—C₂-C₃ alkynyl, —O—, or a bond;
ring C3 is 4- to 16-membered heterocyclyl or absent, wherein the heterocyclyl has 1 or 2 heteroatoms that is N; each R^{C3} is independently C₁-C₆ alkyl; p is 0 or 1; Z⁵ is a bond;
ring C4 is 5 to 14-membered heterocyclyl, C₆-C₁₀ aryl or 5- to 10-membered heteroaryl, wherein the heterocyclyl and the heteroaryl each has 1 or 2 heteroatoms that is N; each R^{C4} is independently C₁-C₆ alkyl, halo, or oxo; q is 0, 1 or 2; Z³ is a bond; and Z⁴ is —CH— or N.

In some embodiments, the present disclosure provides the compound of Formula (I), (Ia), and/or (Ib), or a pharmaceutically acceptable salt thereof, wherein the compound is disclosed in any one of the Examples disclosed herein. In some embodiments, the present disclosure provides the compound of Formula (I), (Ia), and/or (Ib), or a pharmaceutically acceptable salt thereof, wherein the compound has the structure:

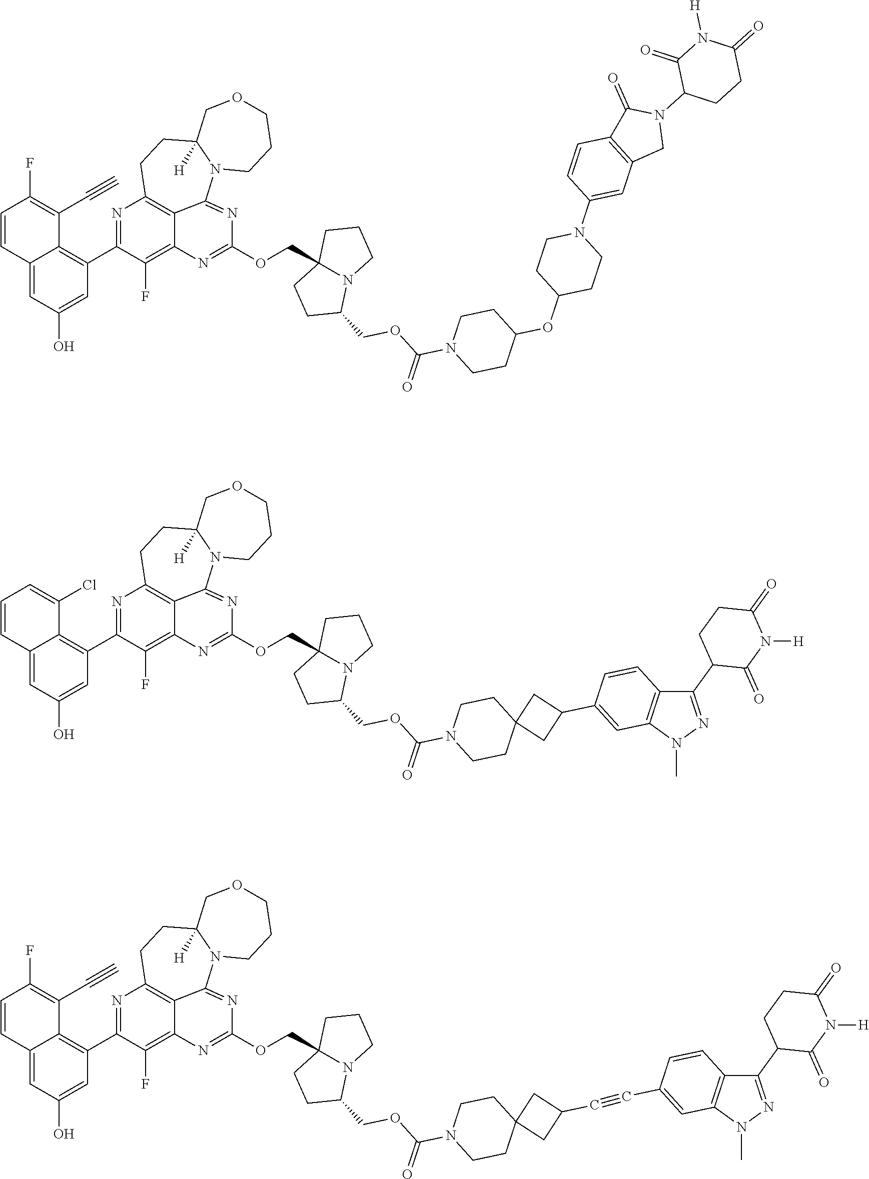

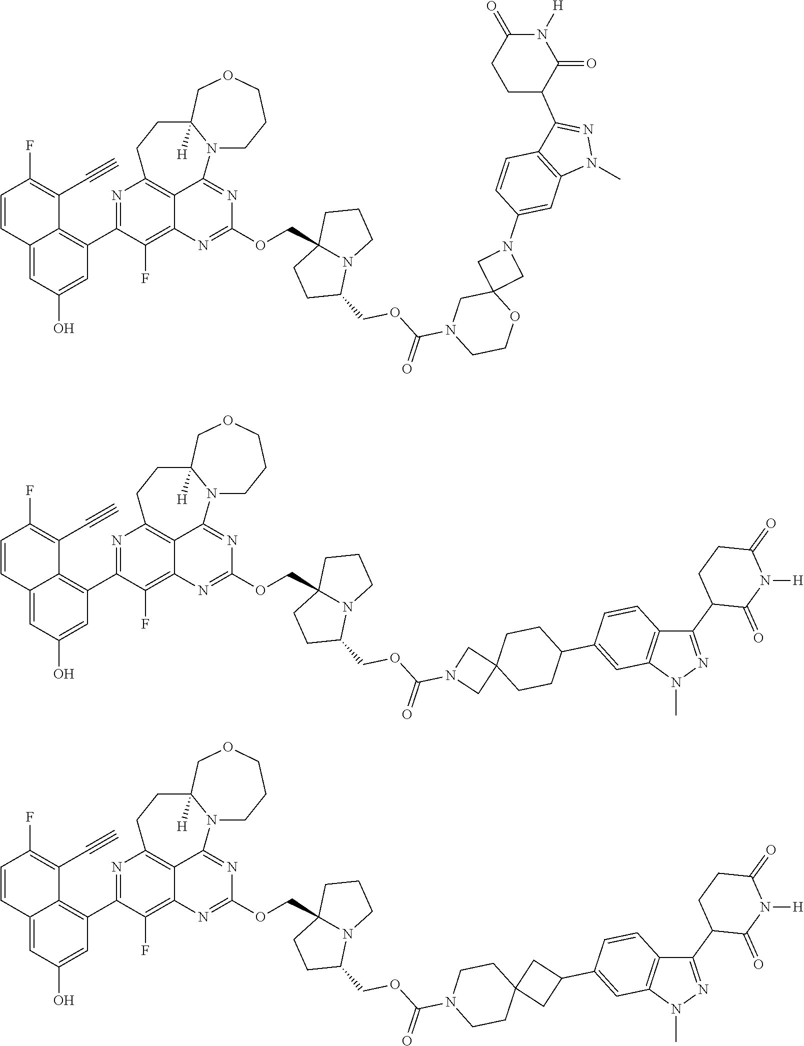

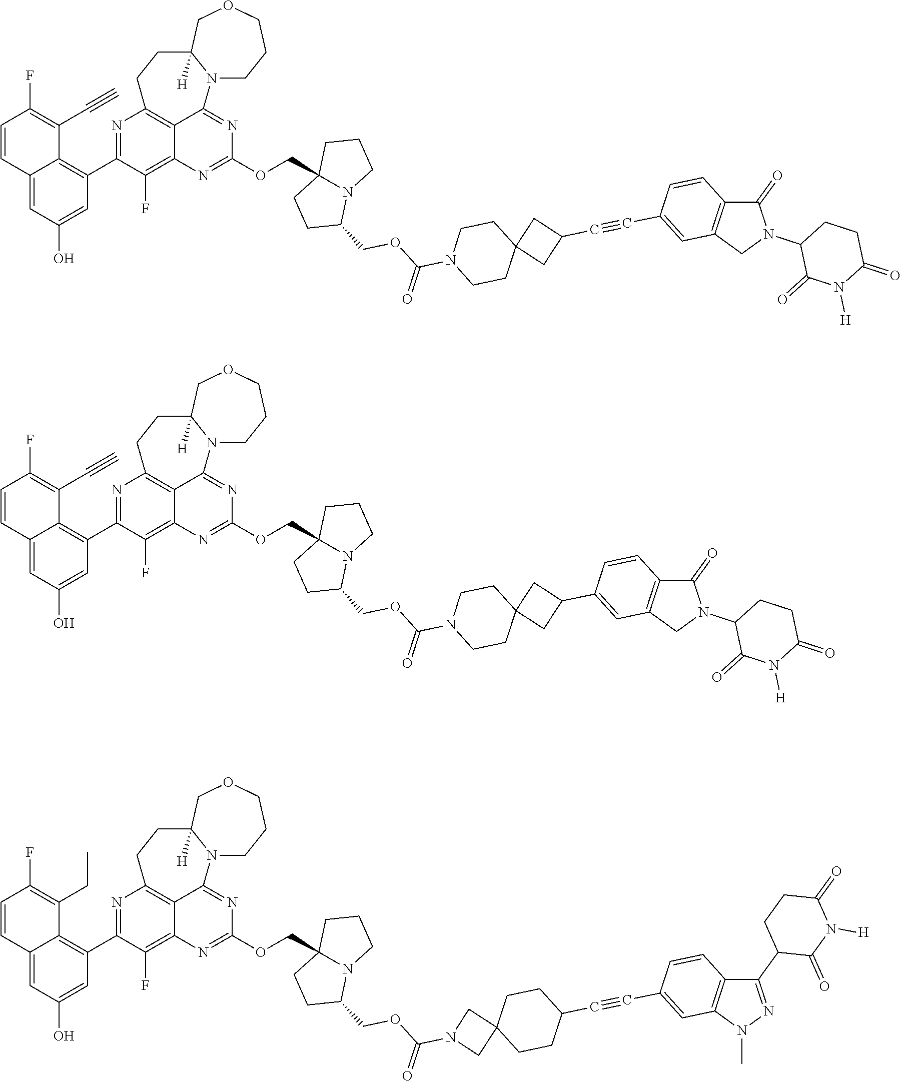

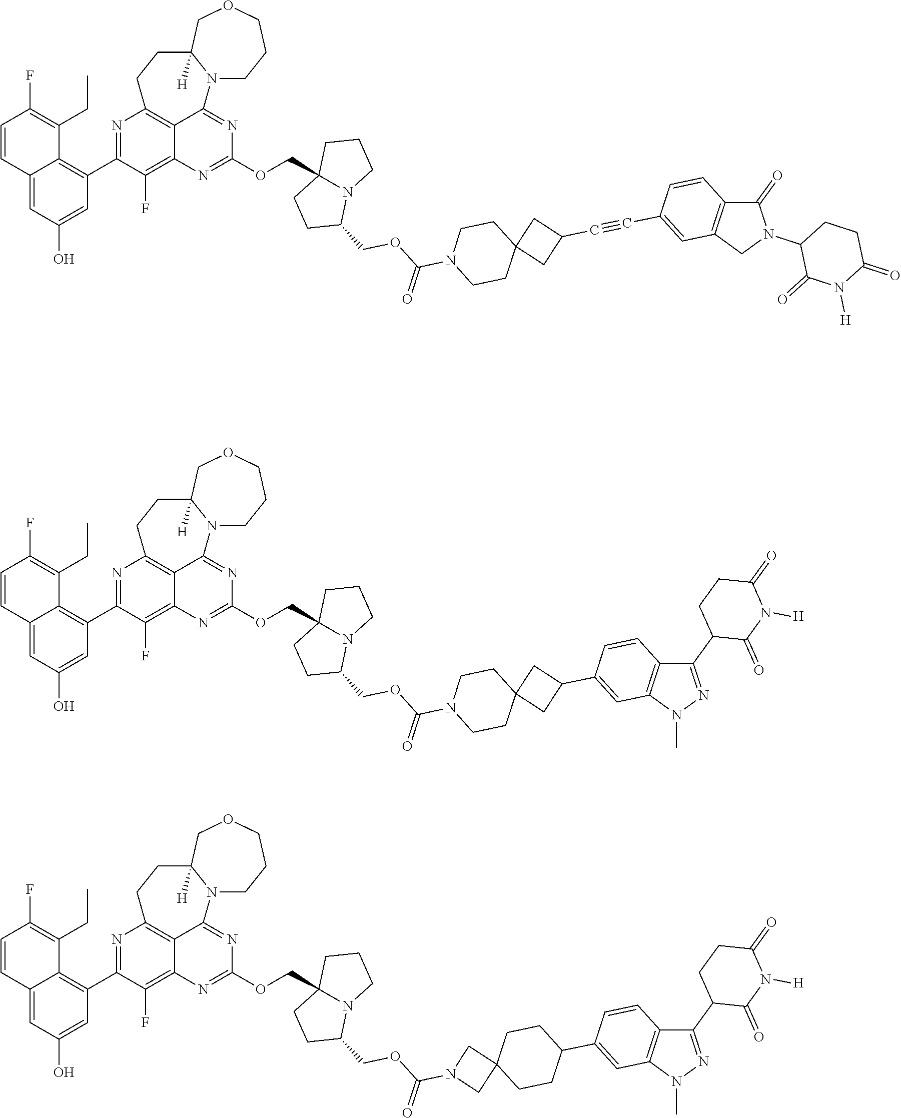

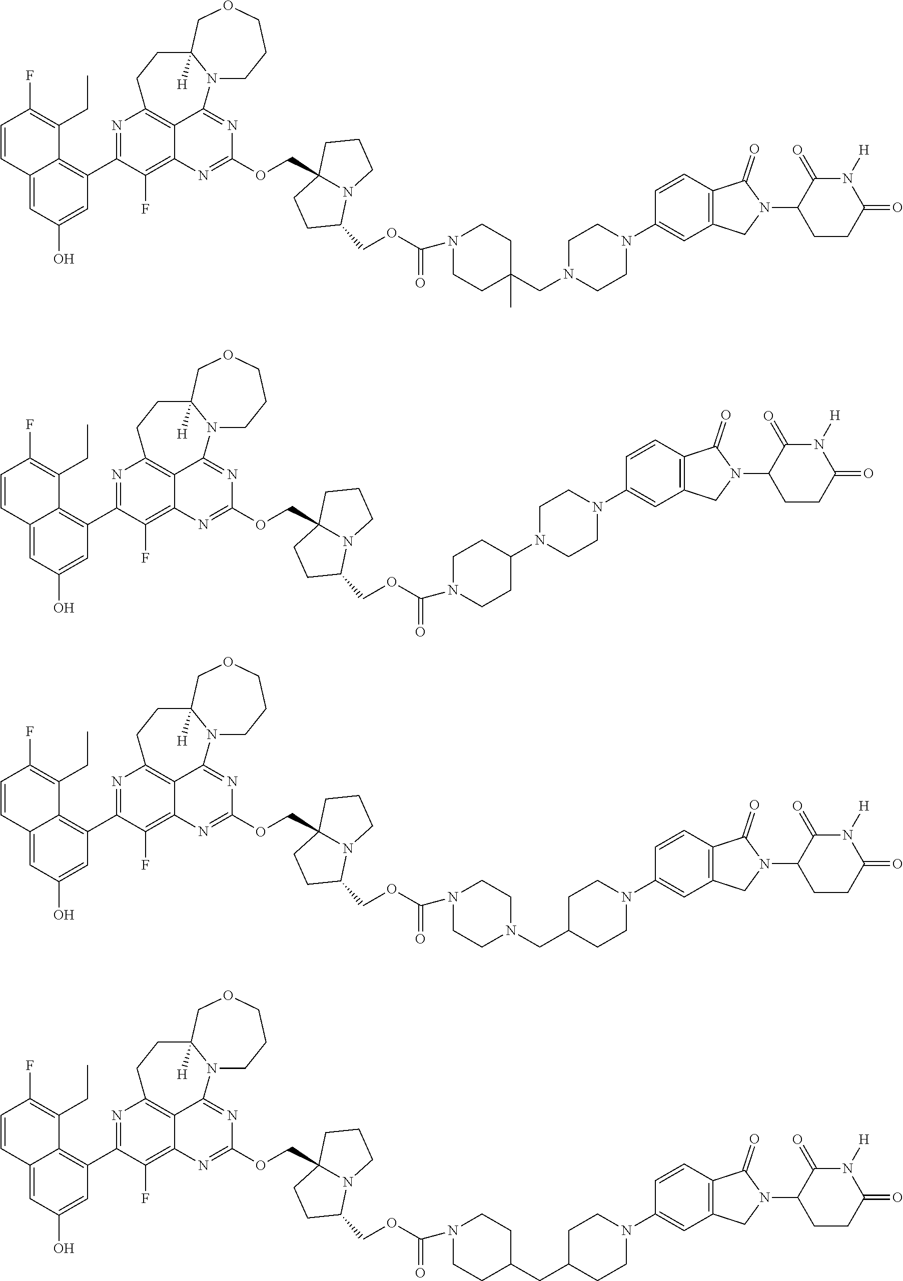

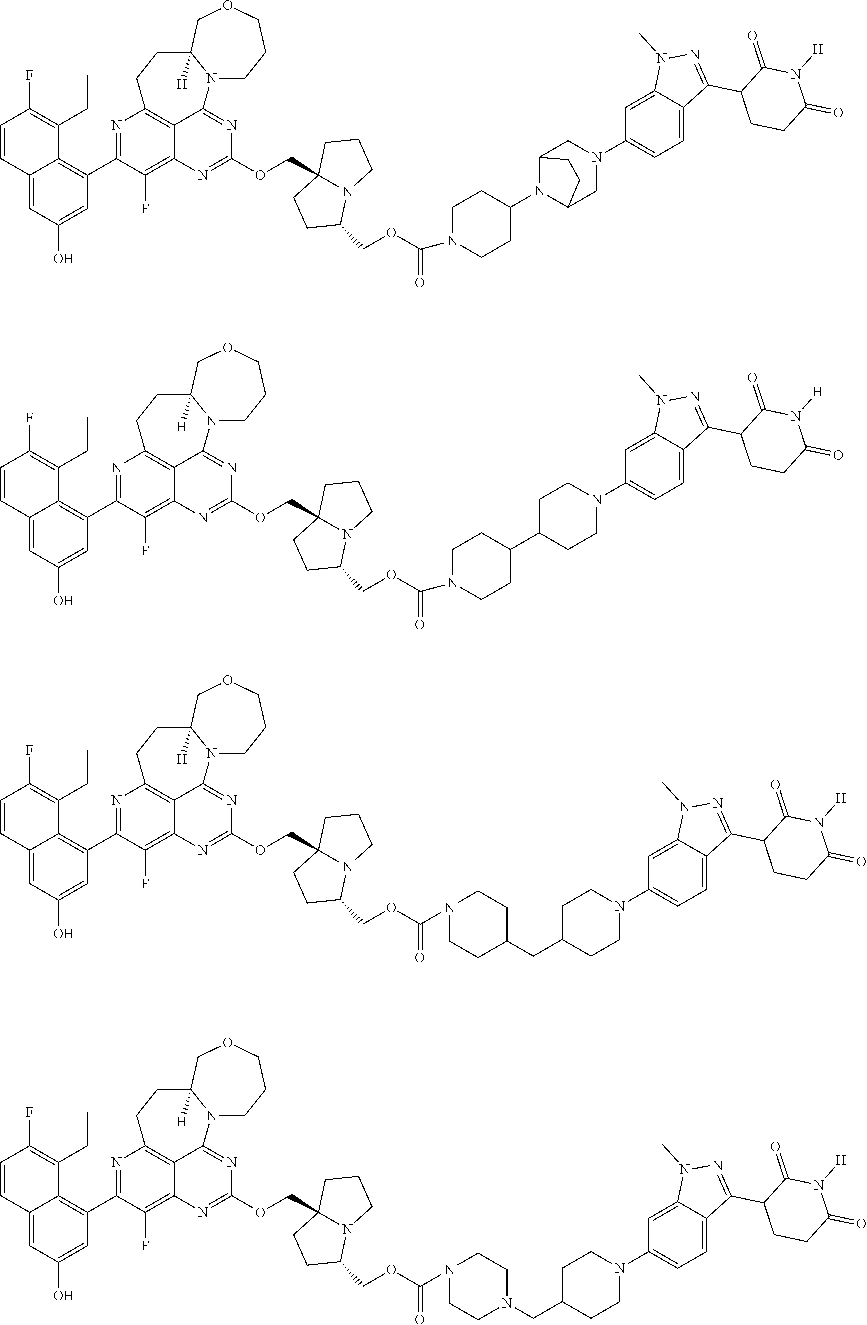

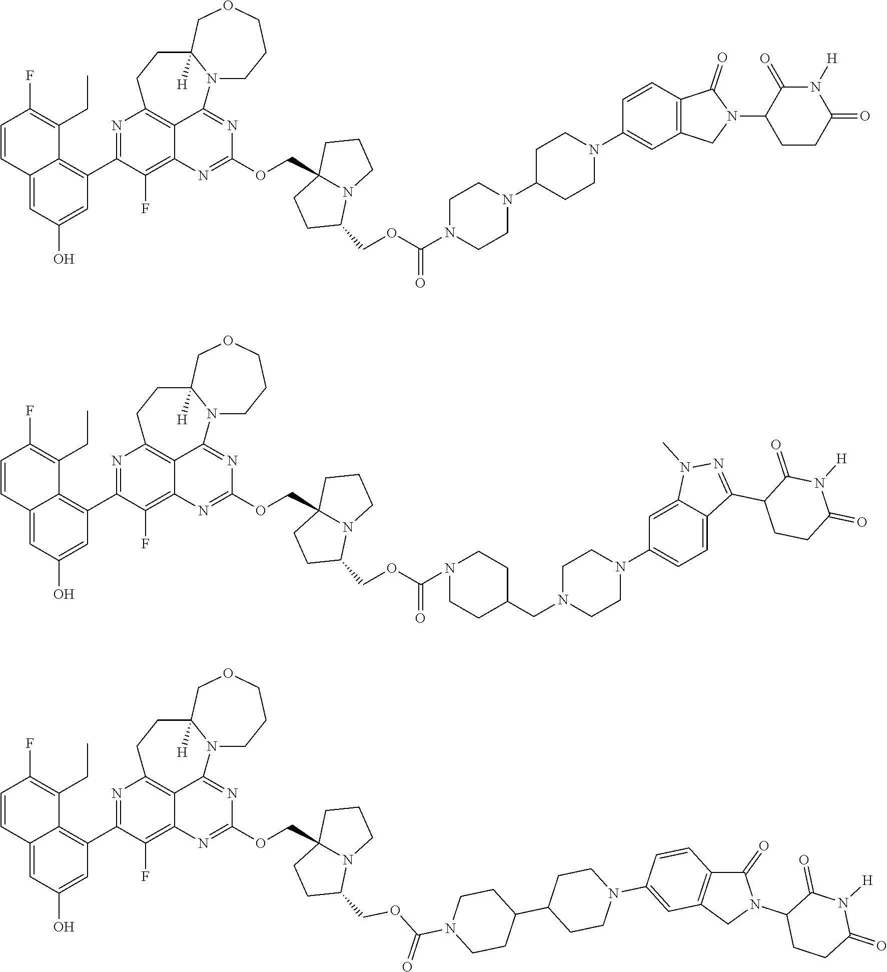

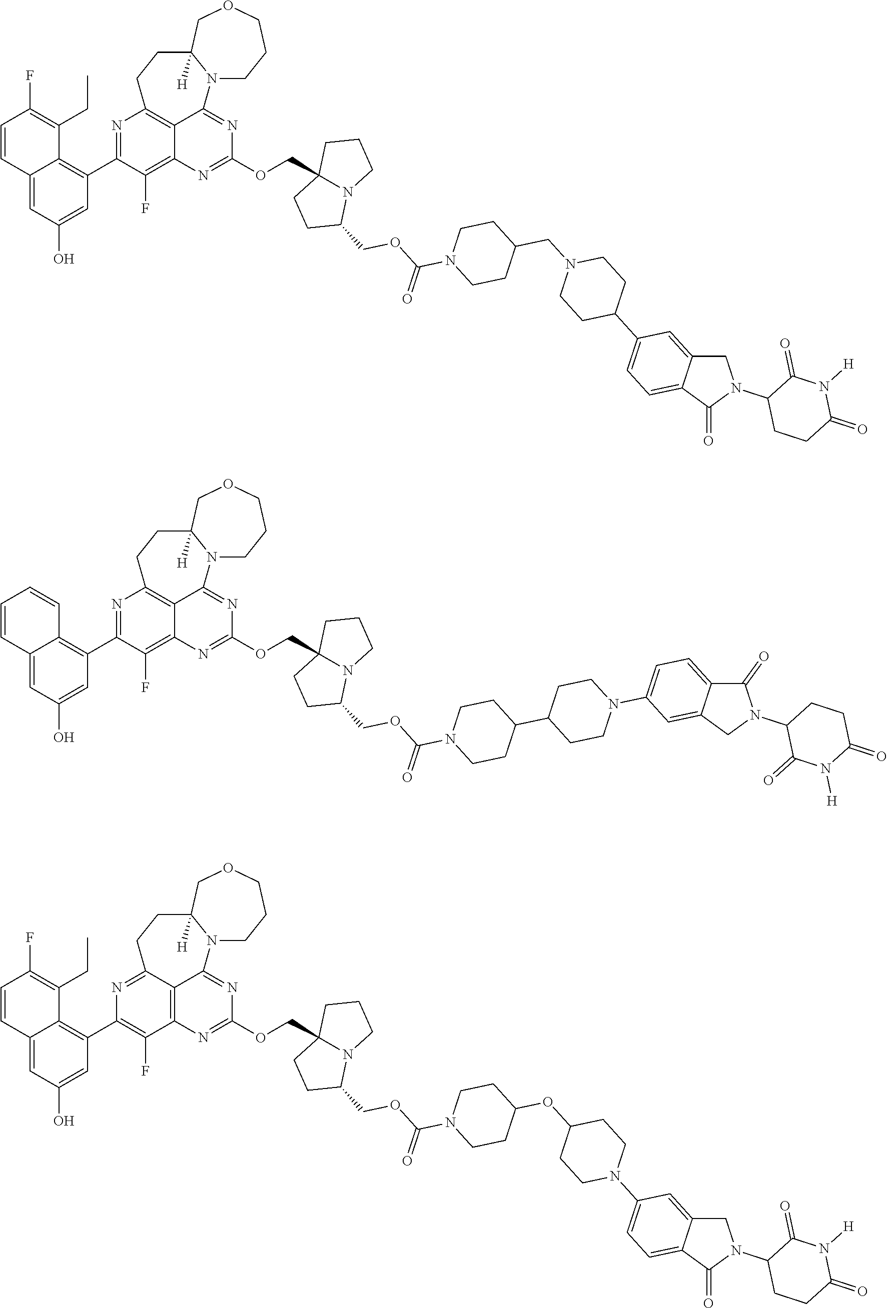

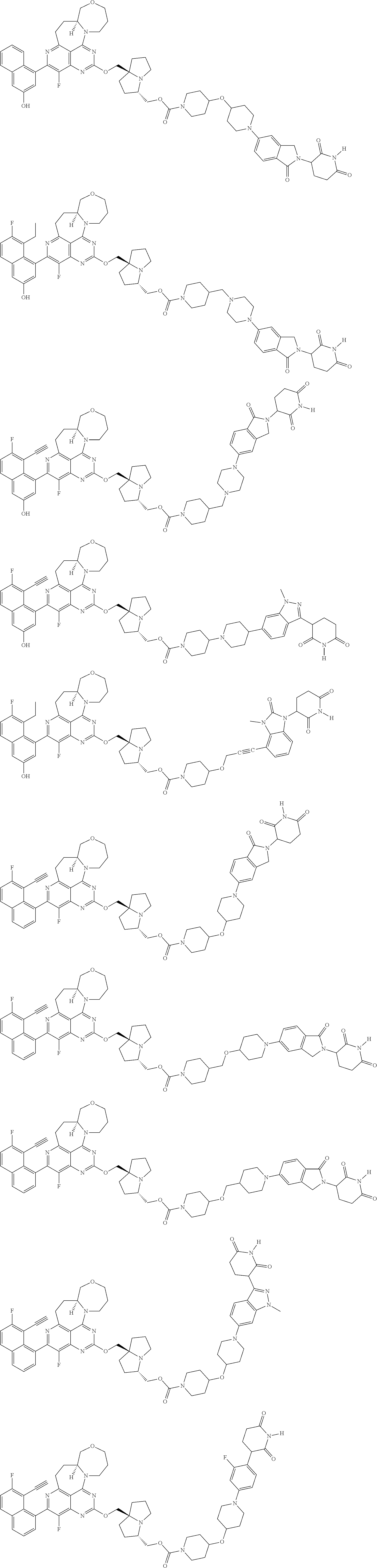

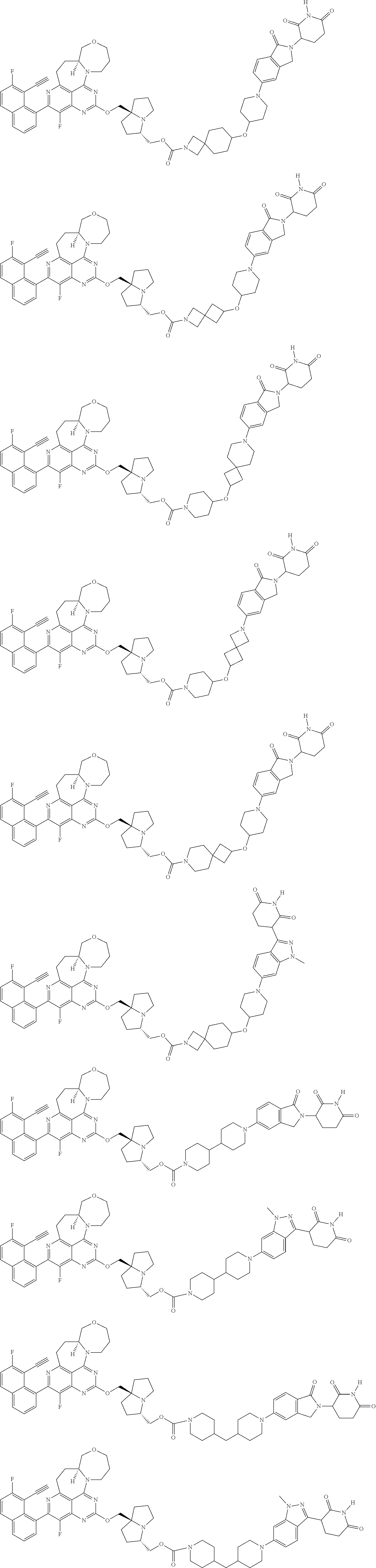

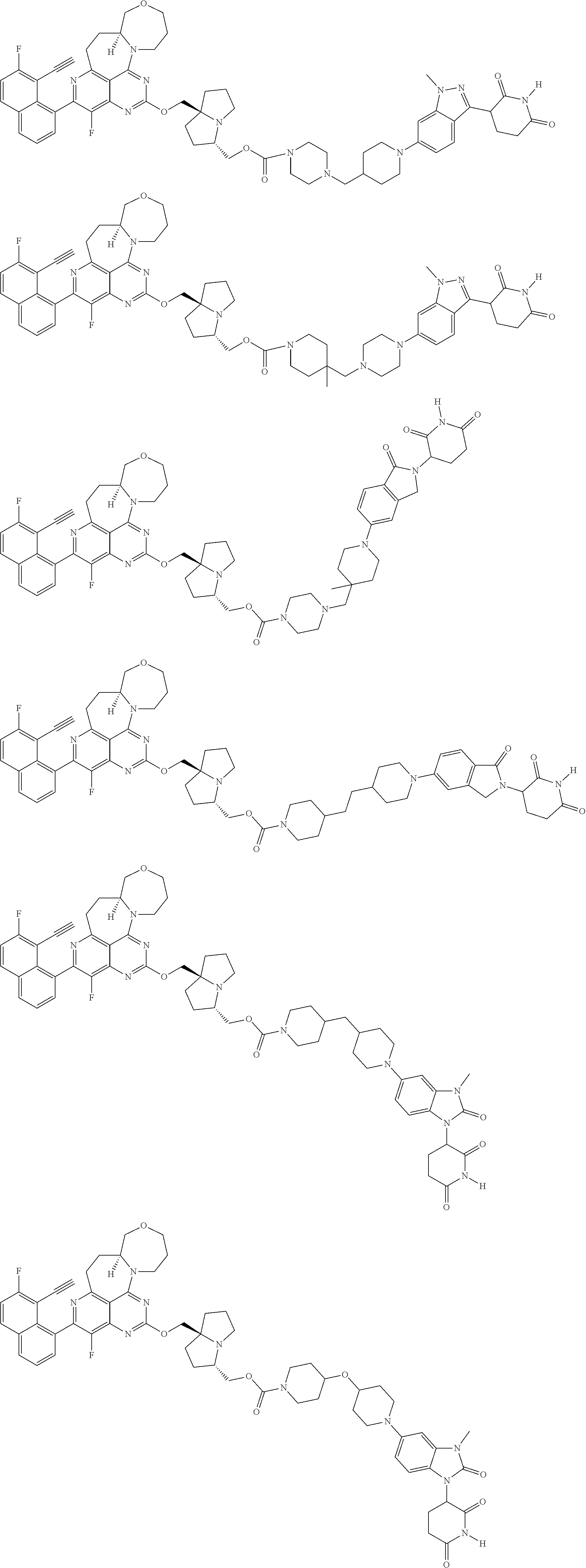

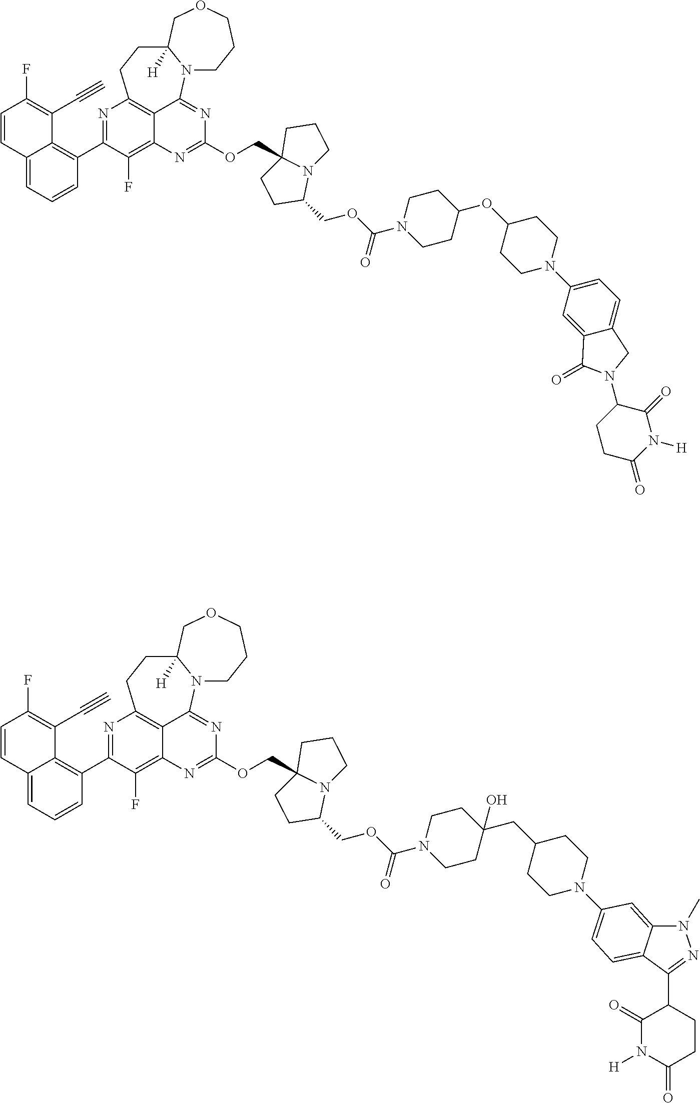

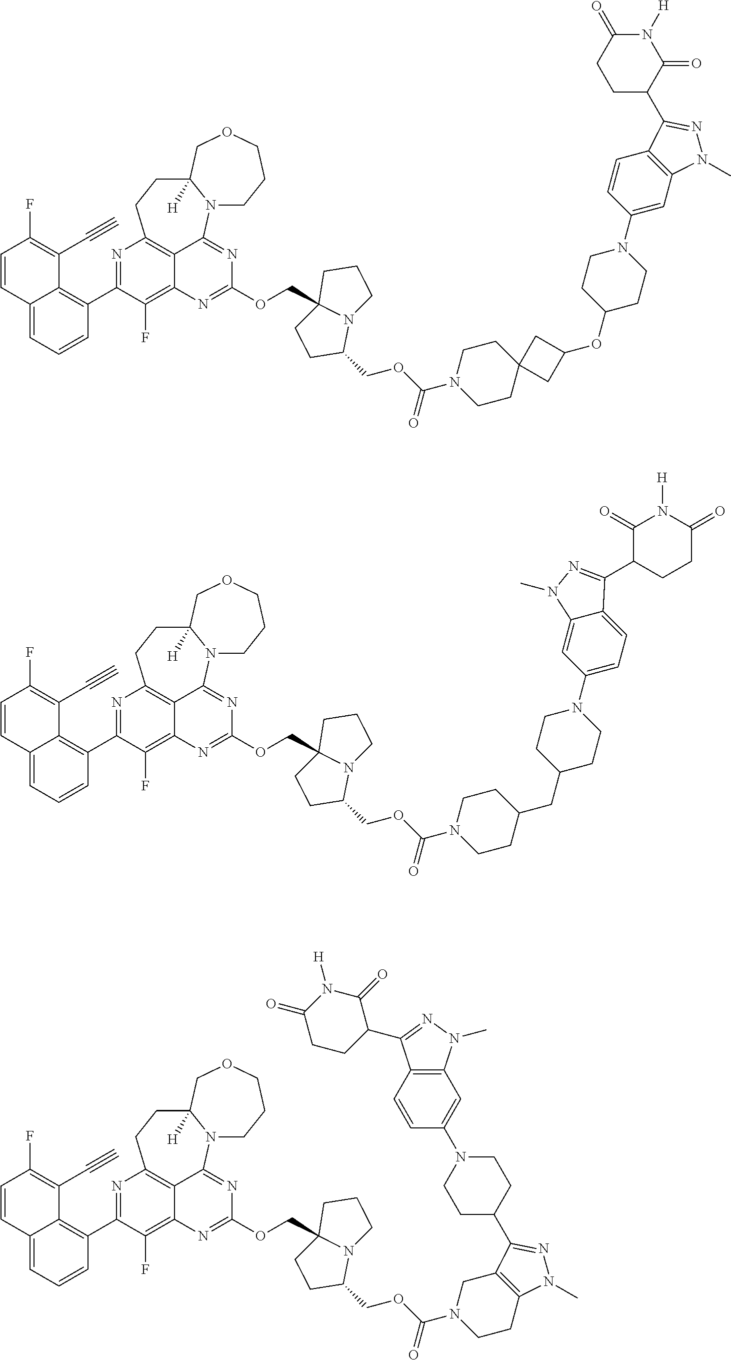

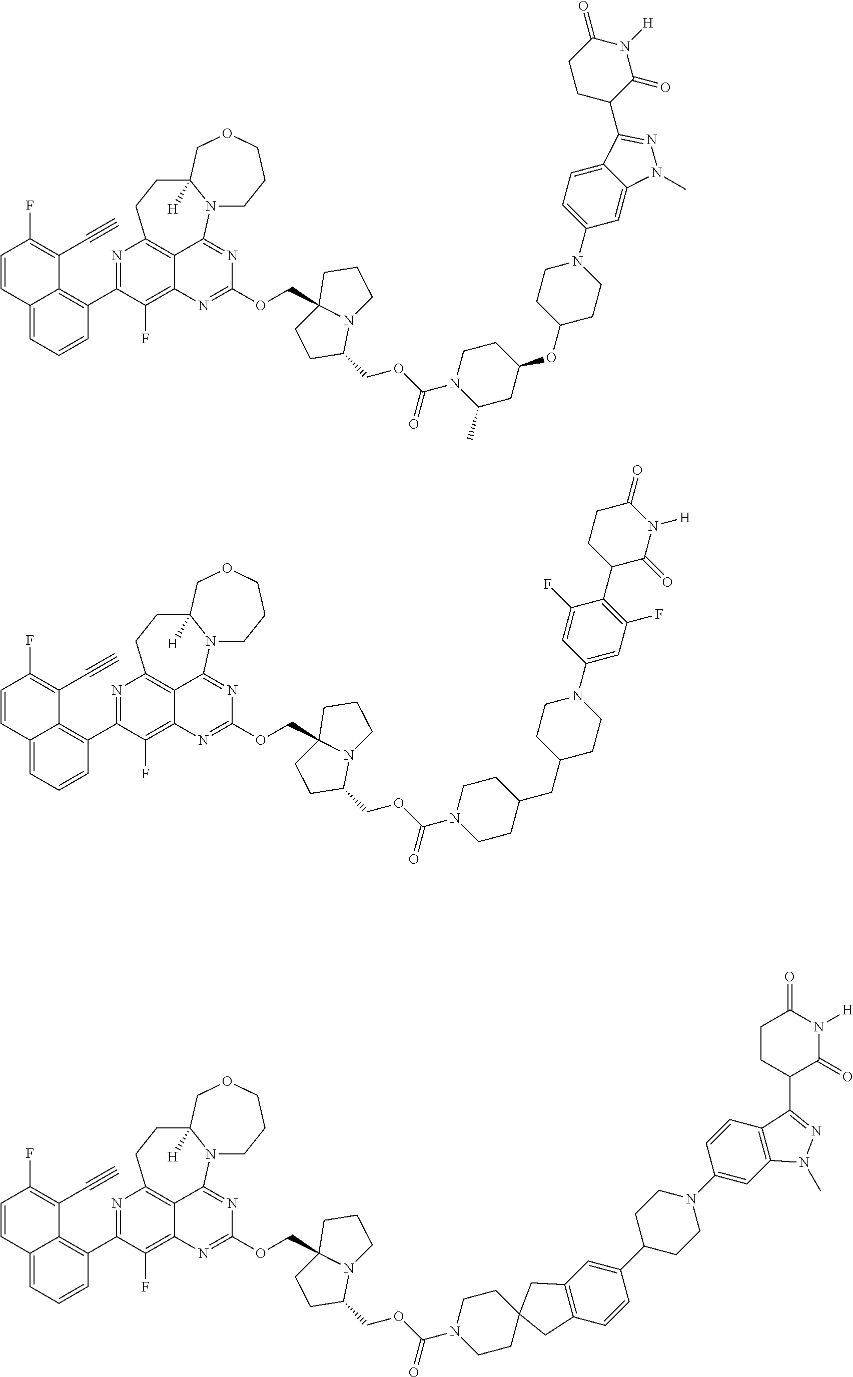

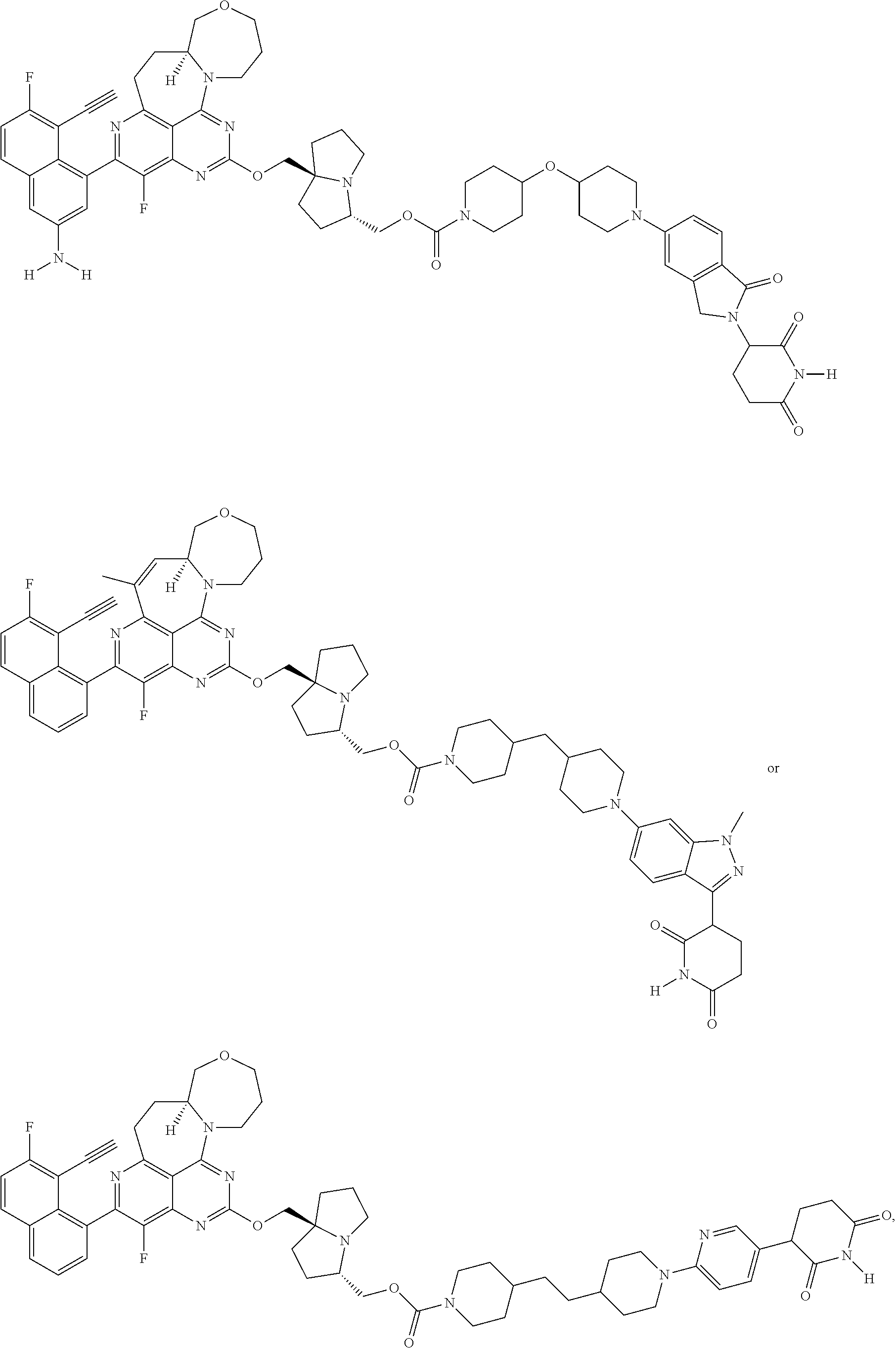

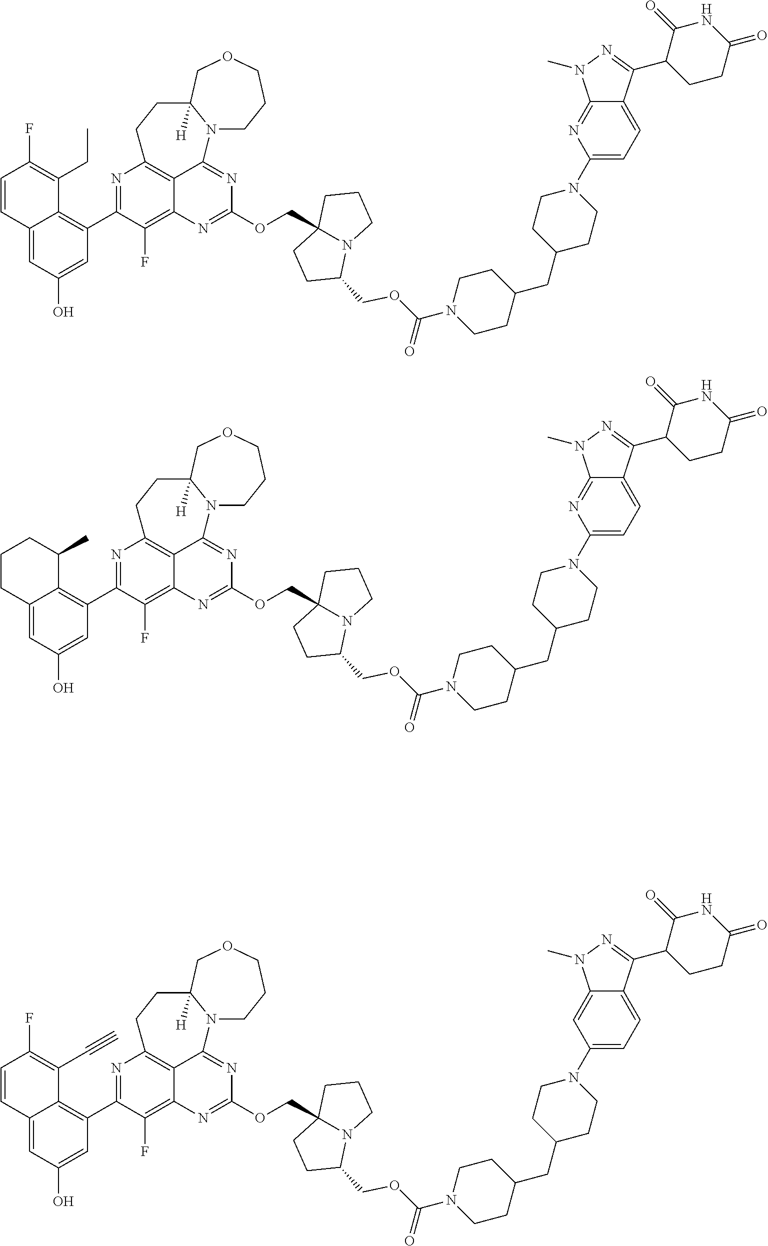

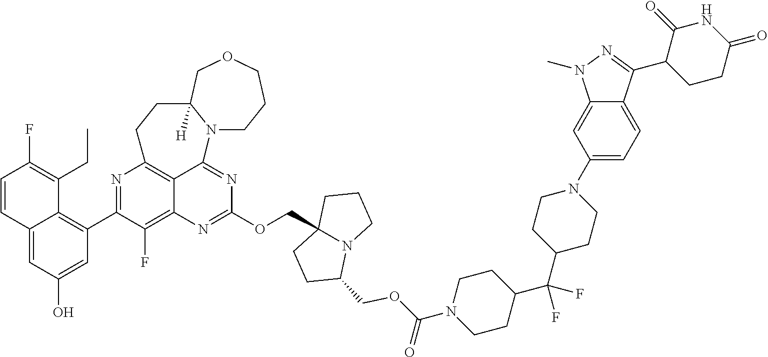

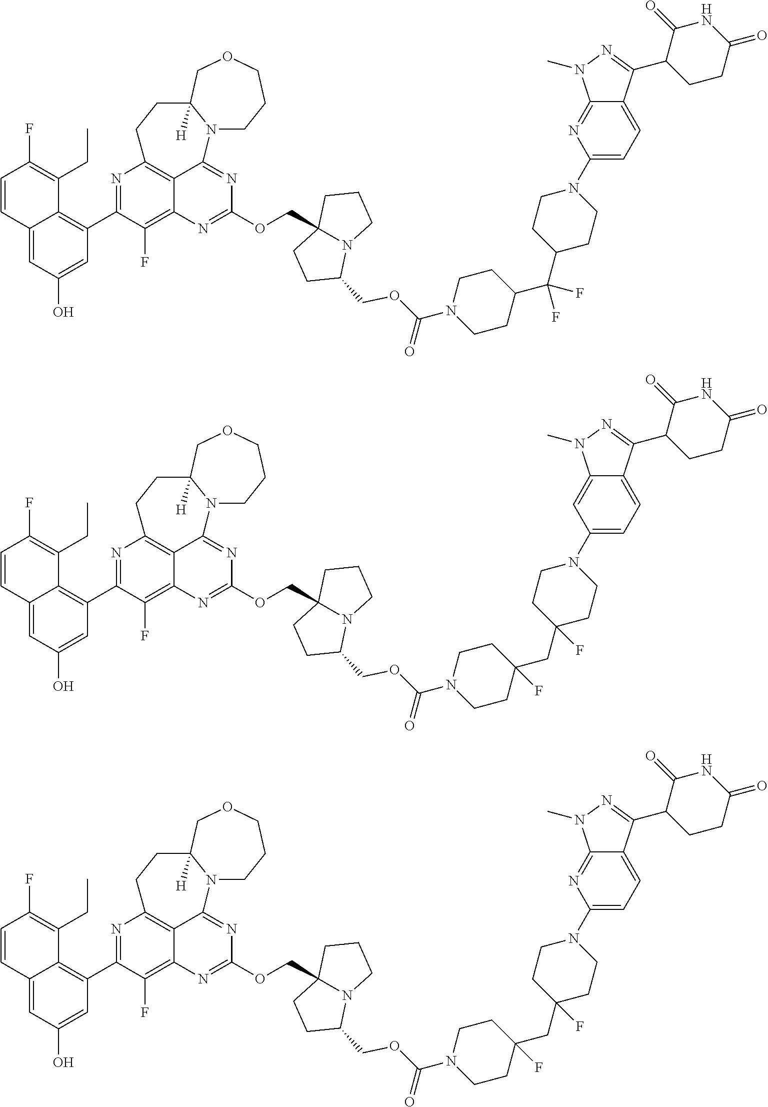

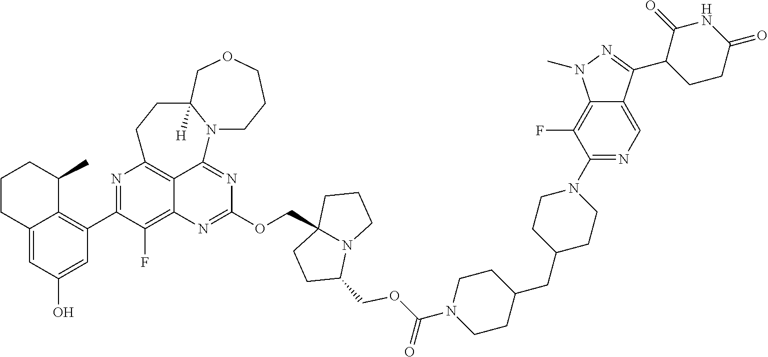

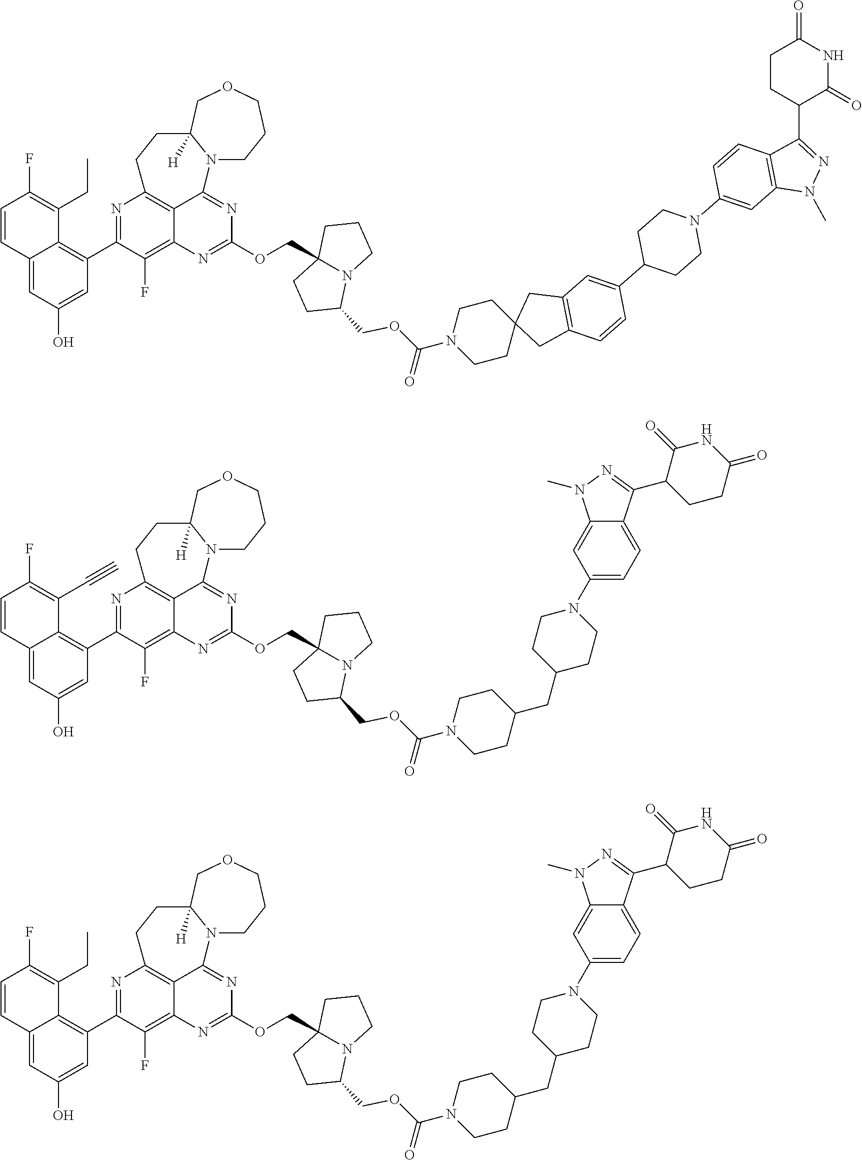

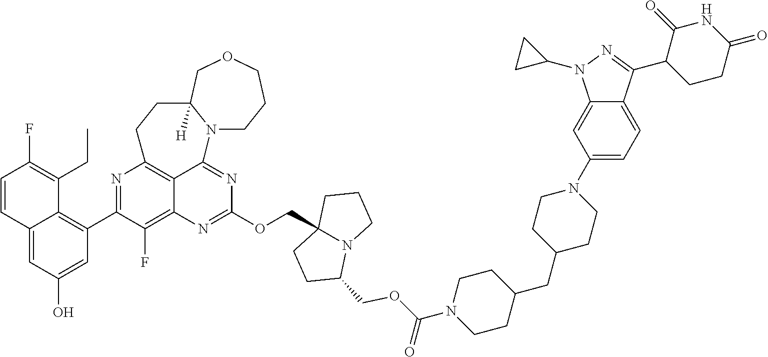

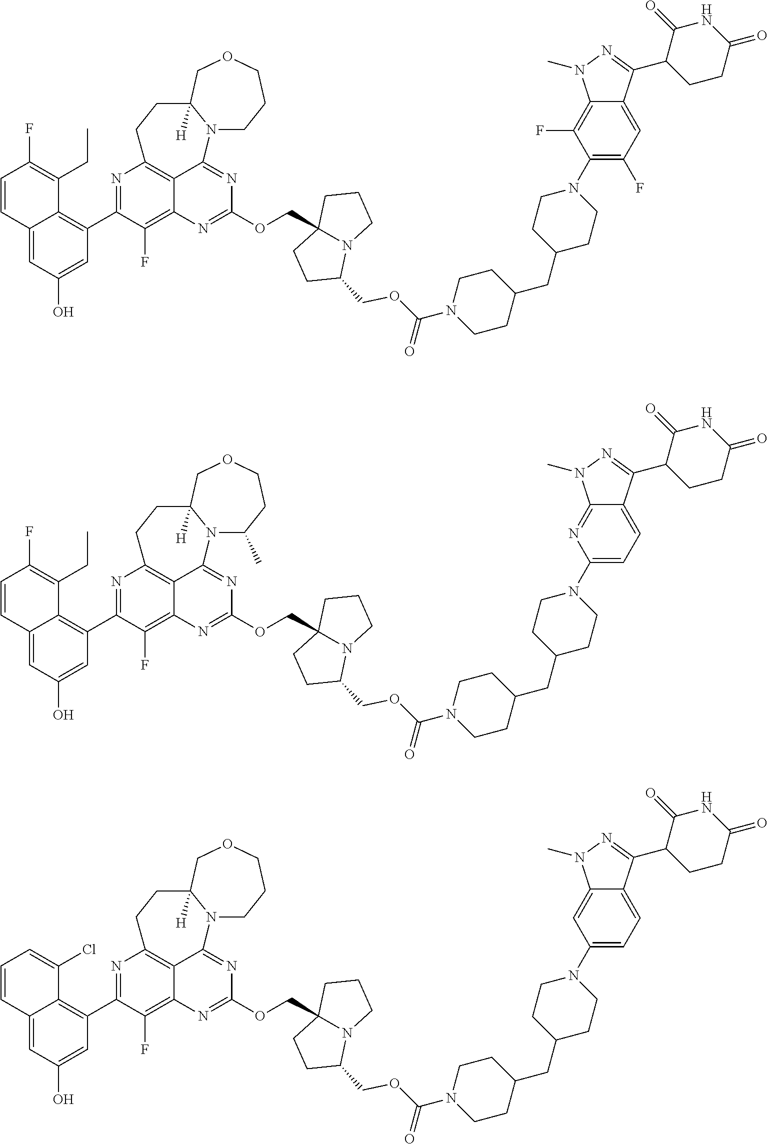

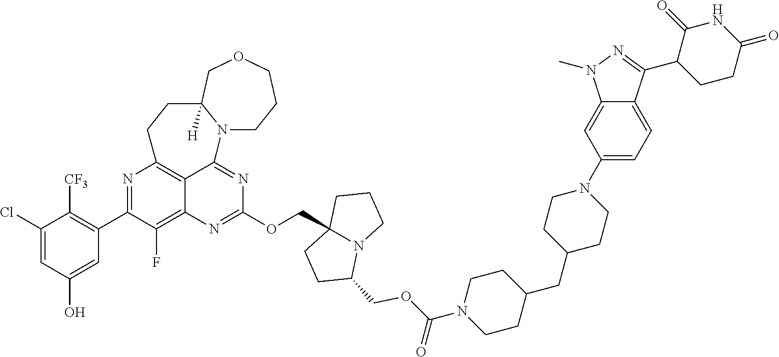

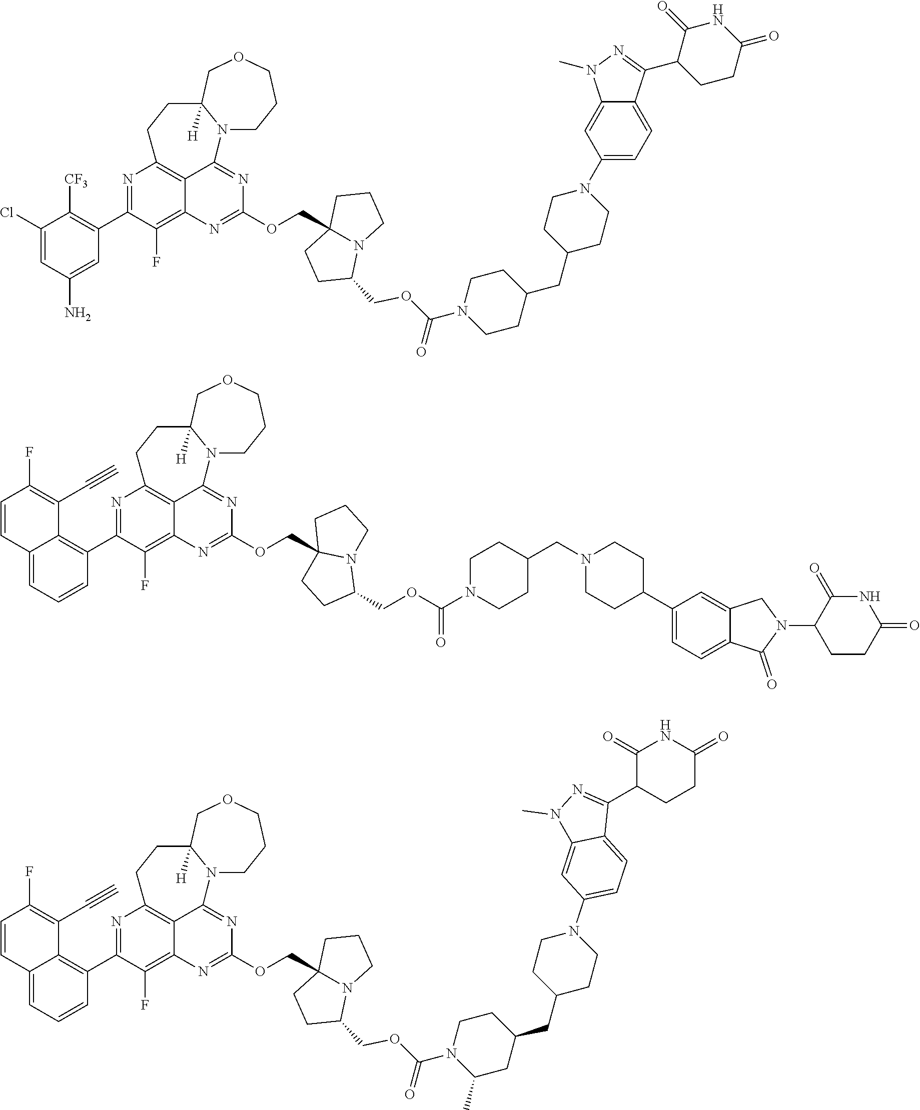

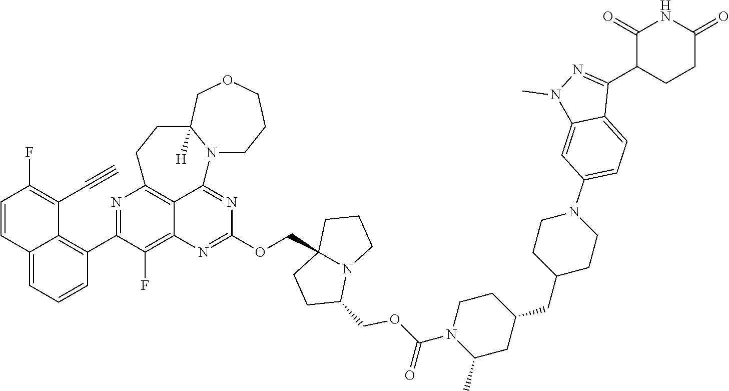

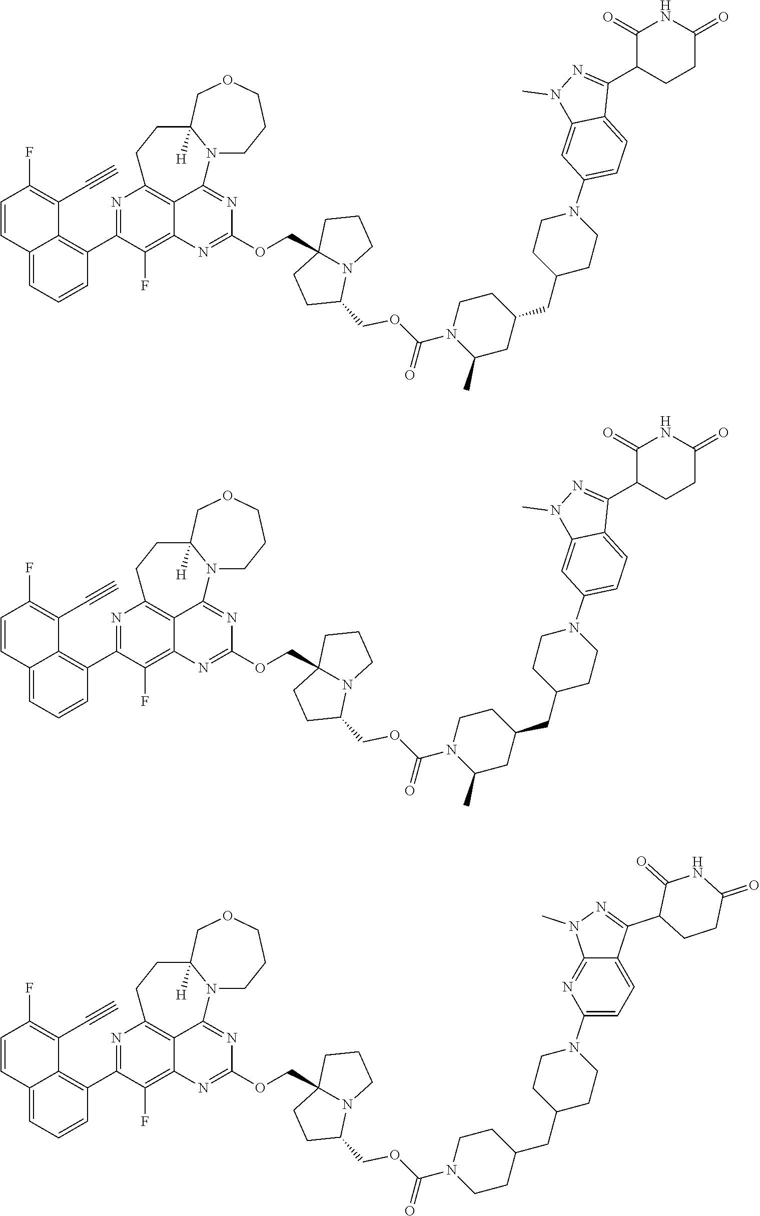

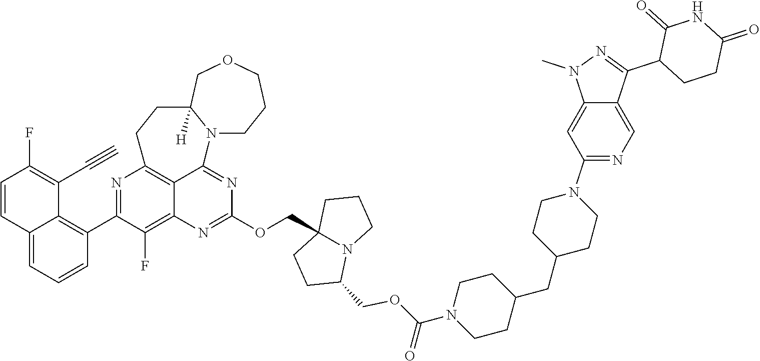

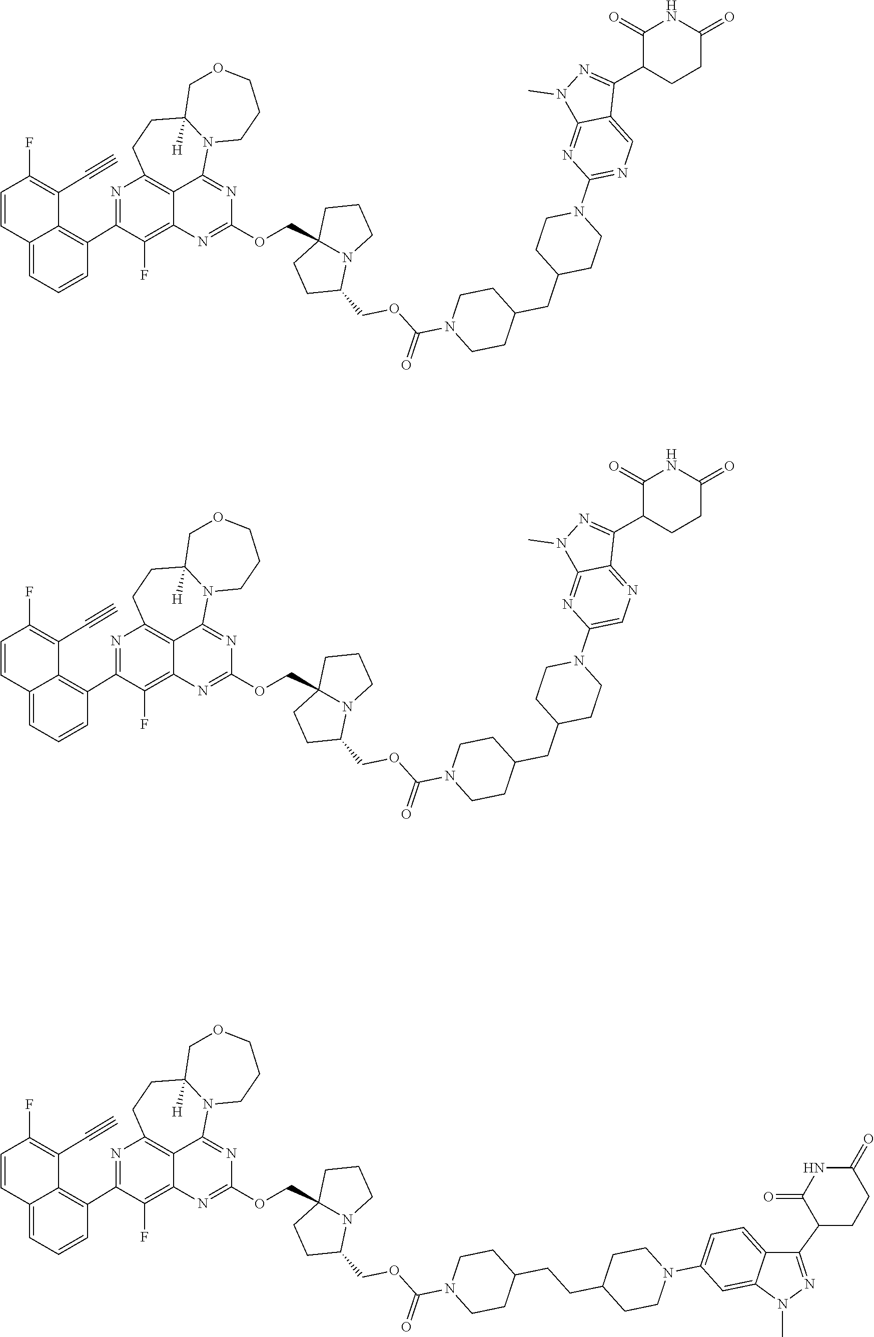

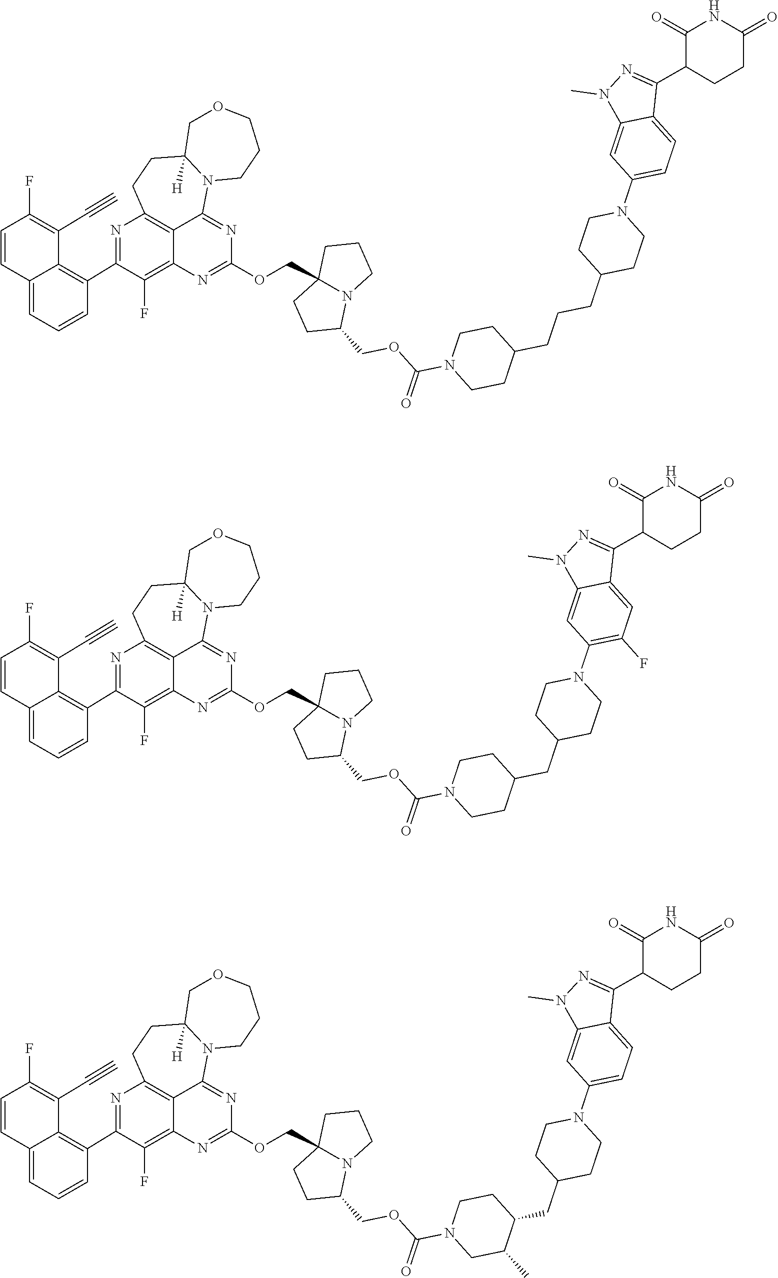

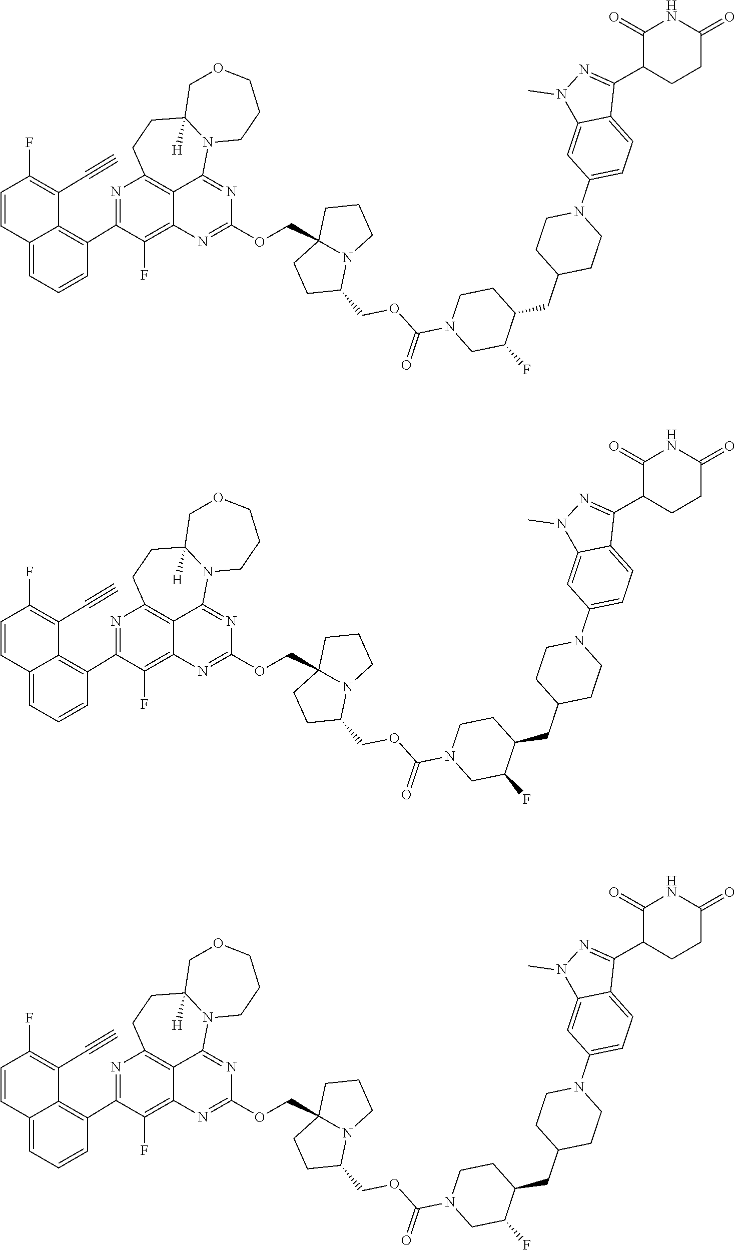

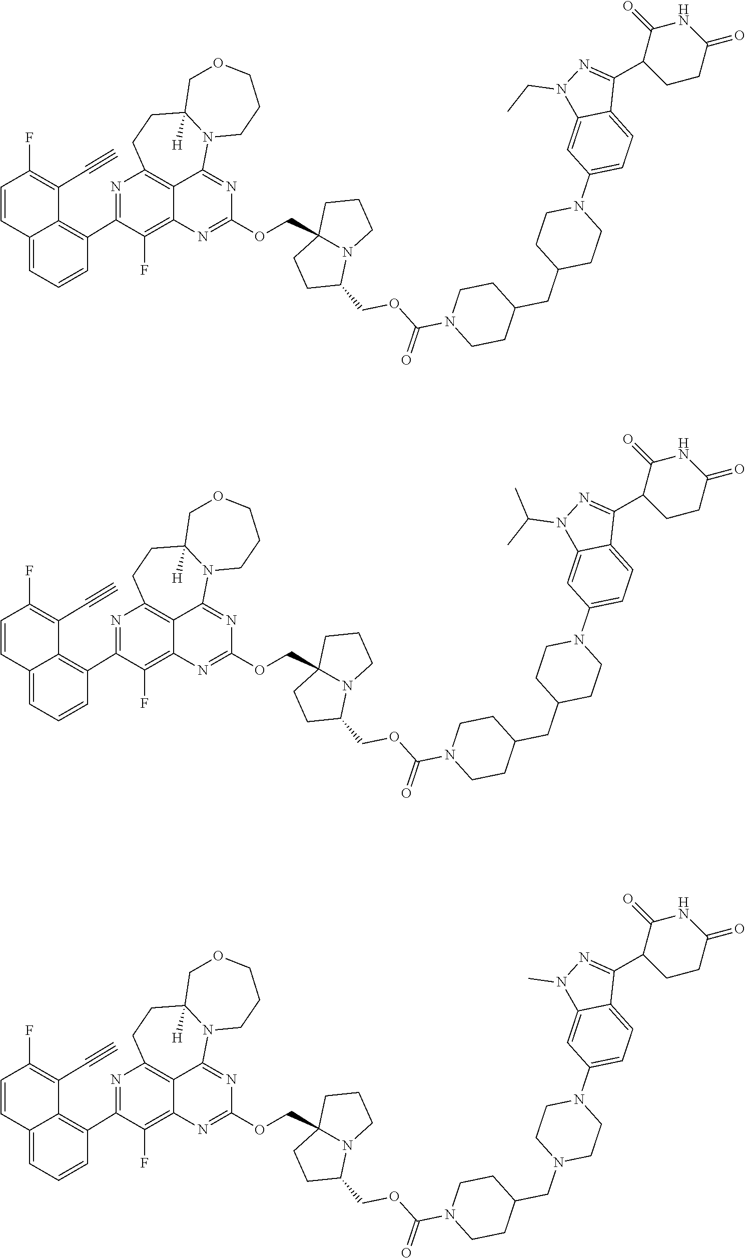

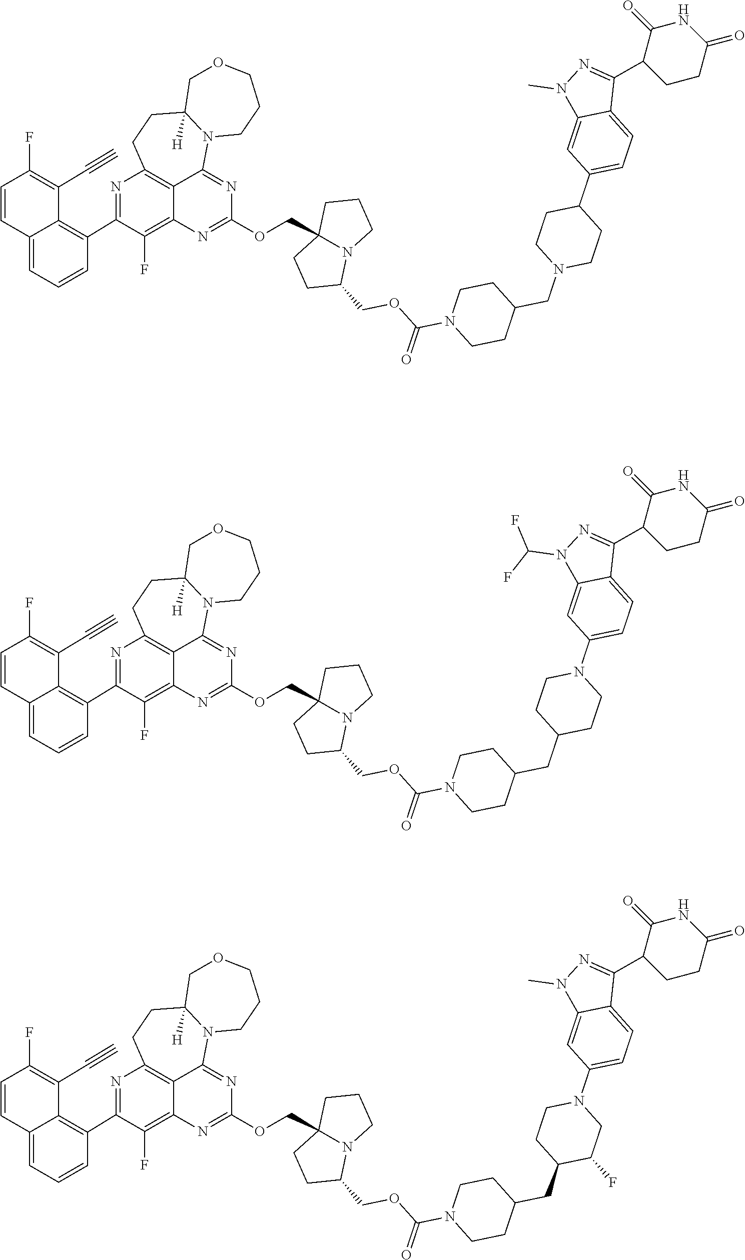

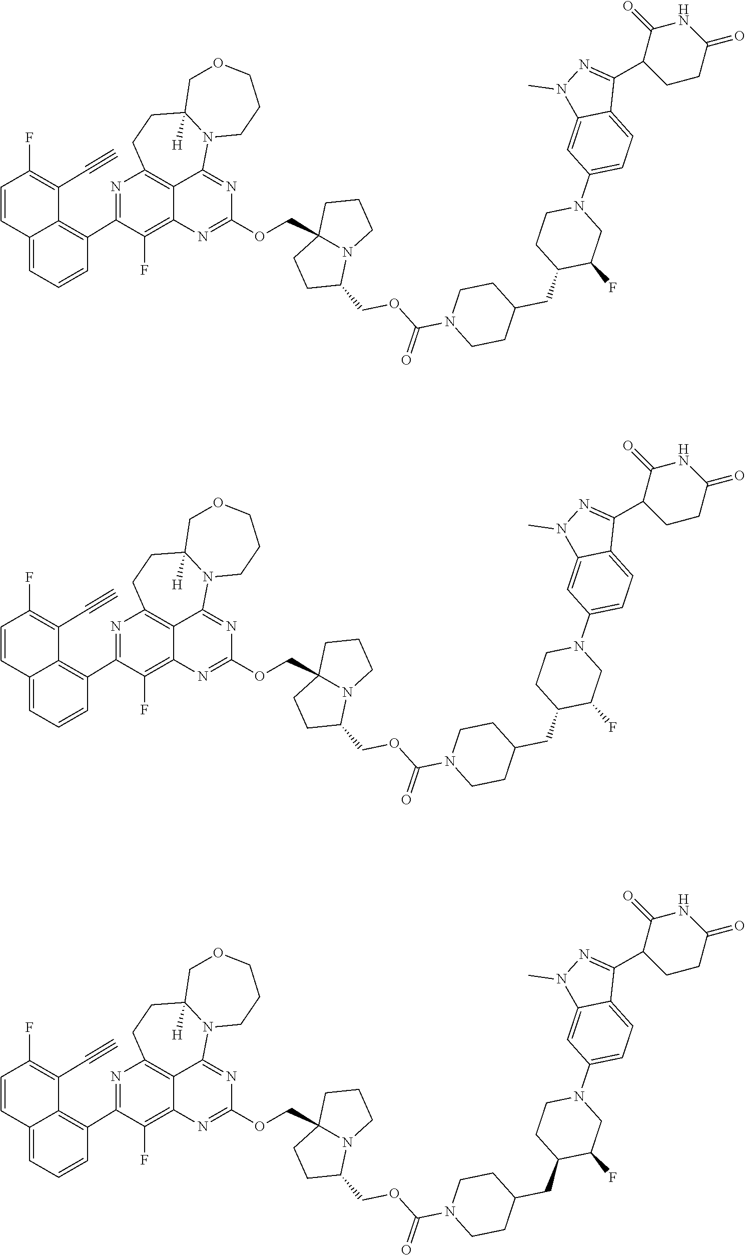

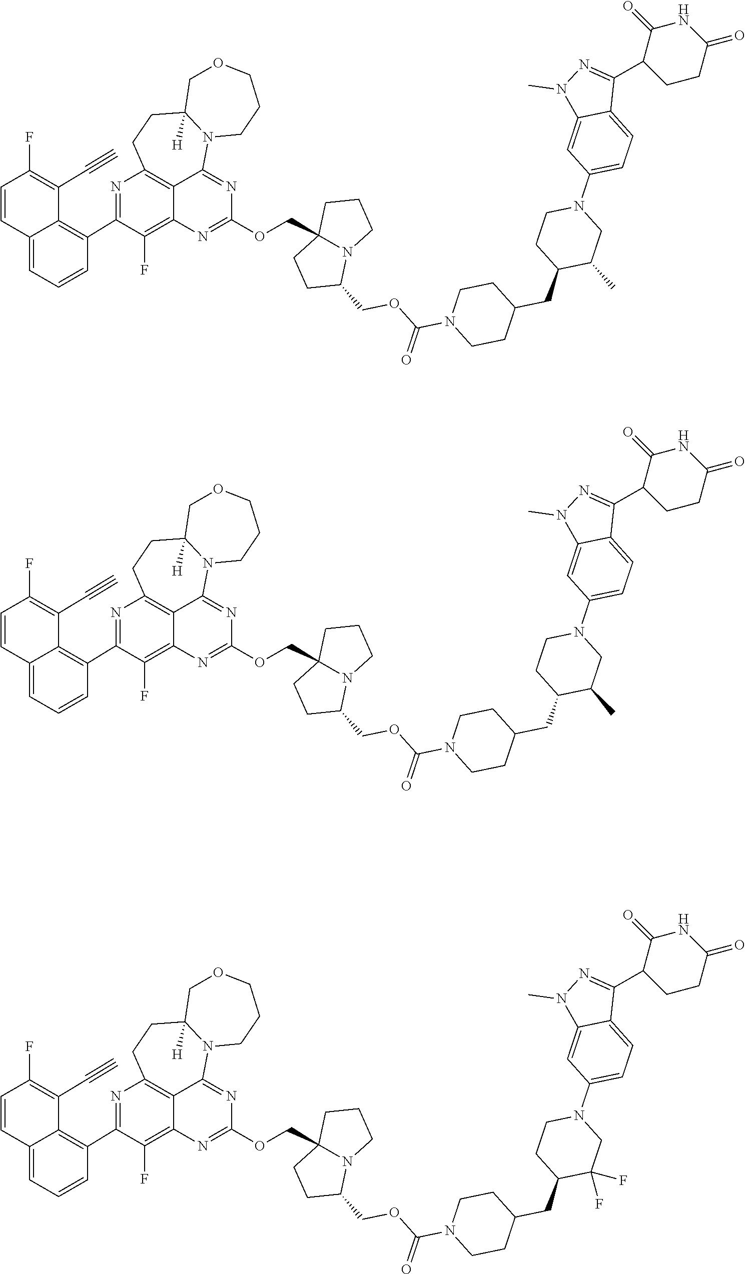

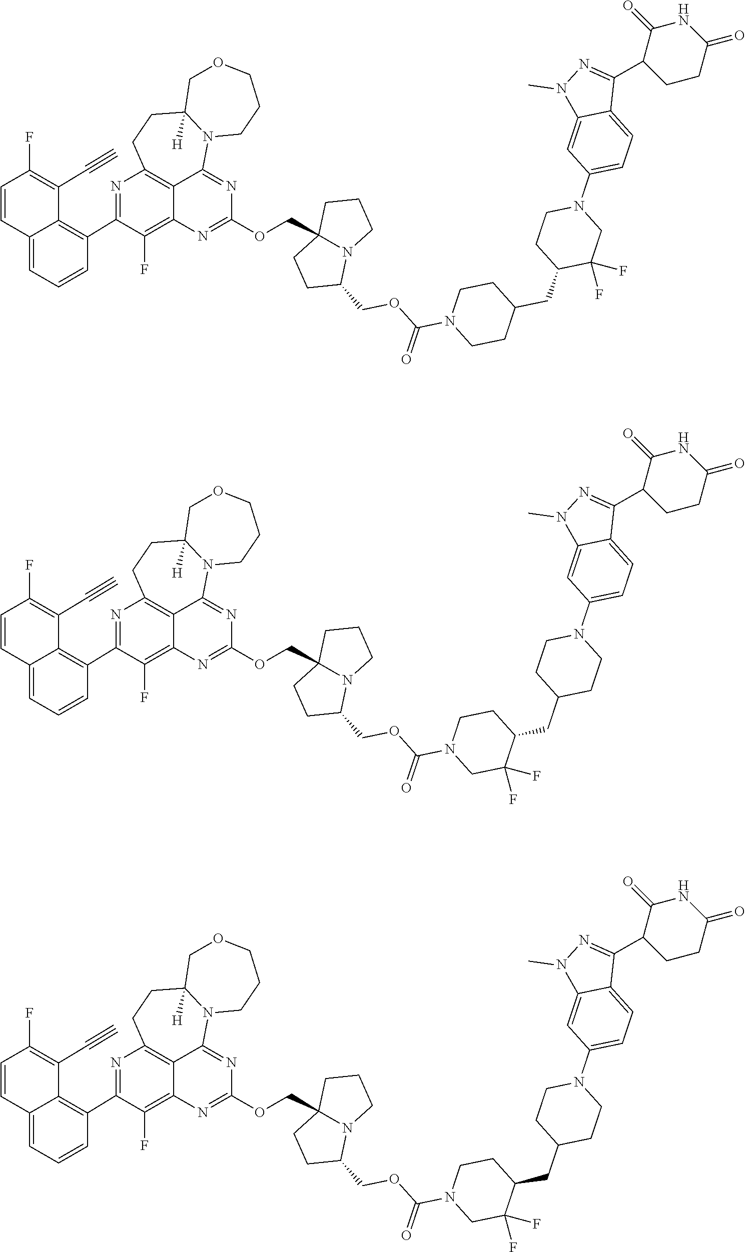

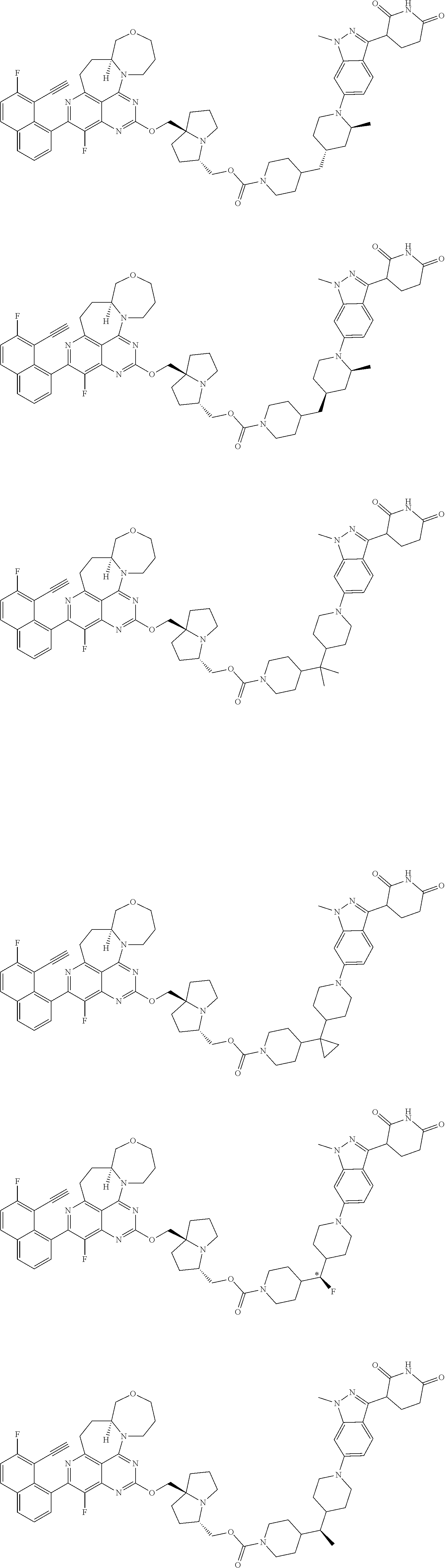

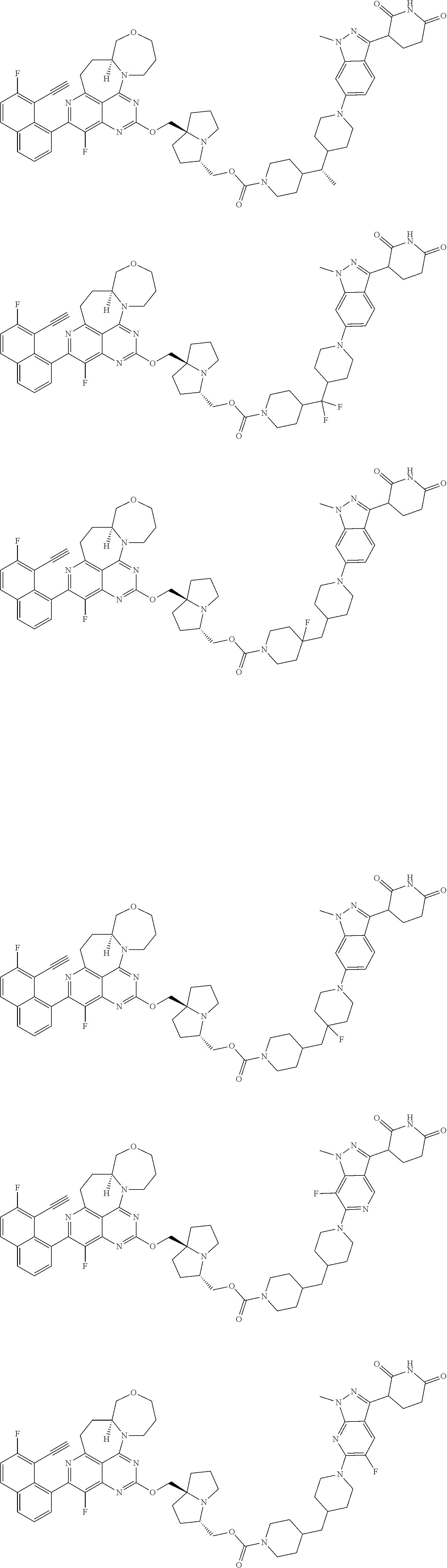

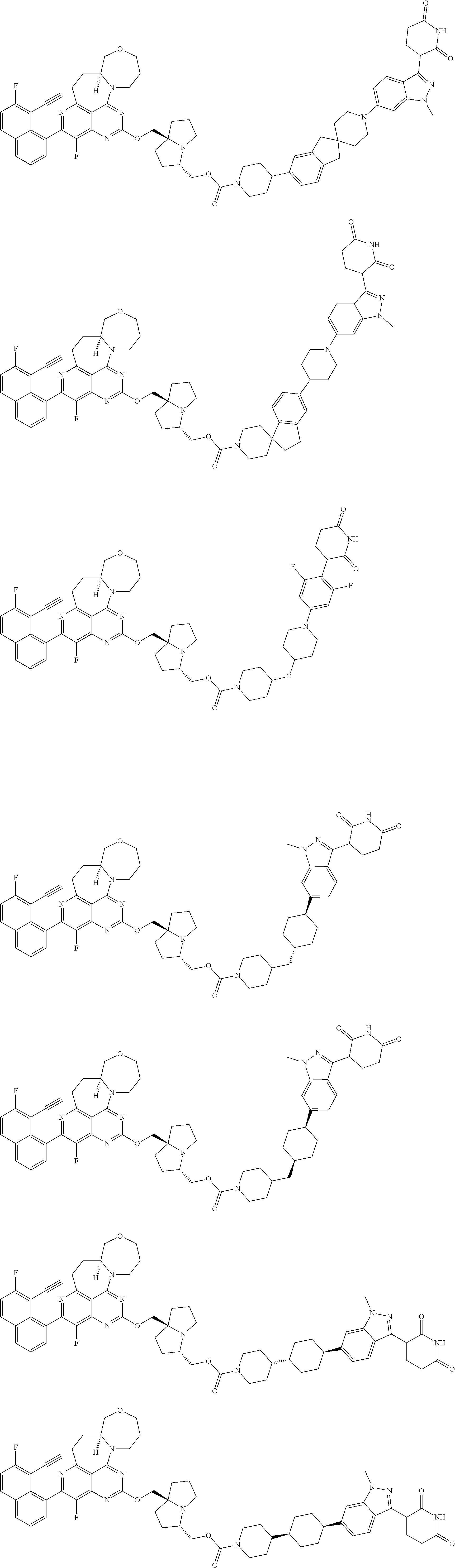

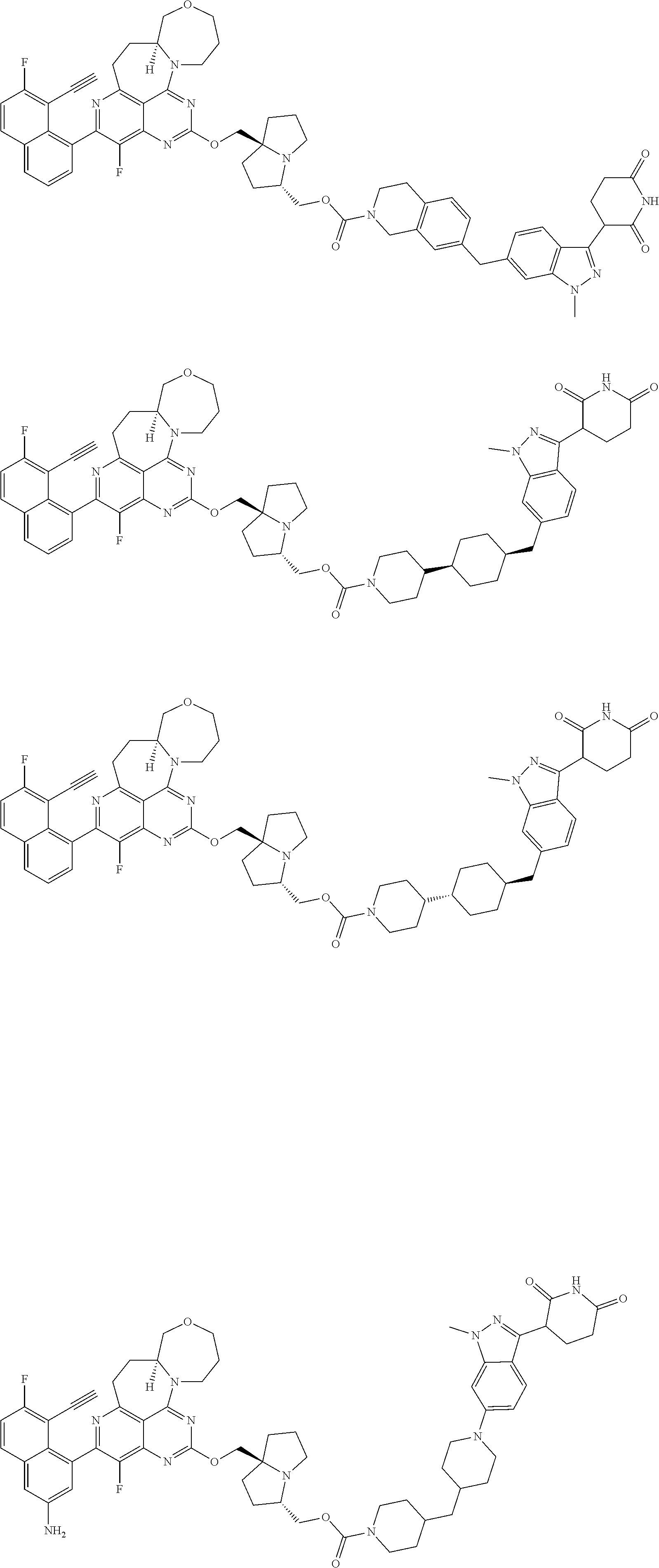

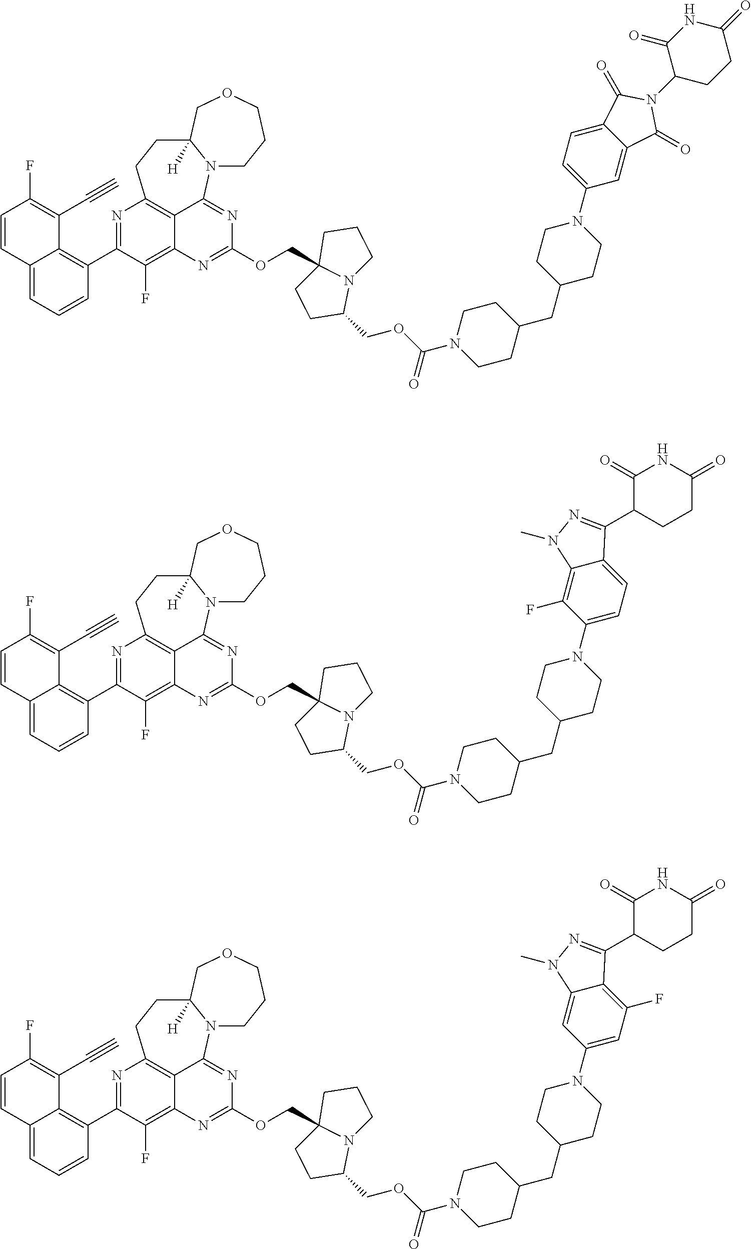

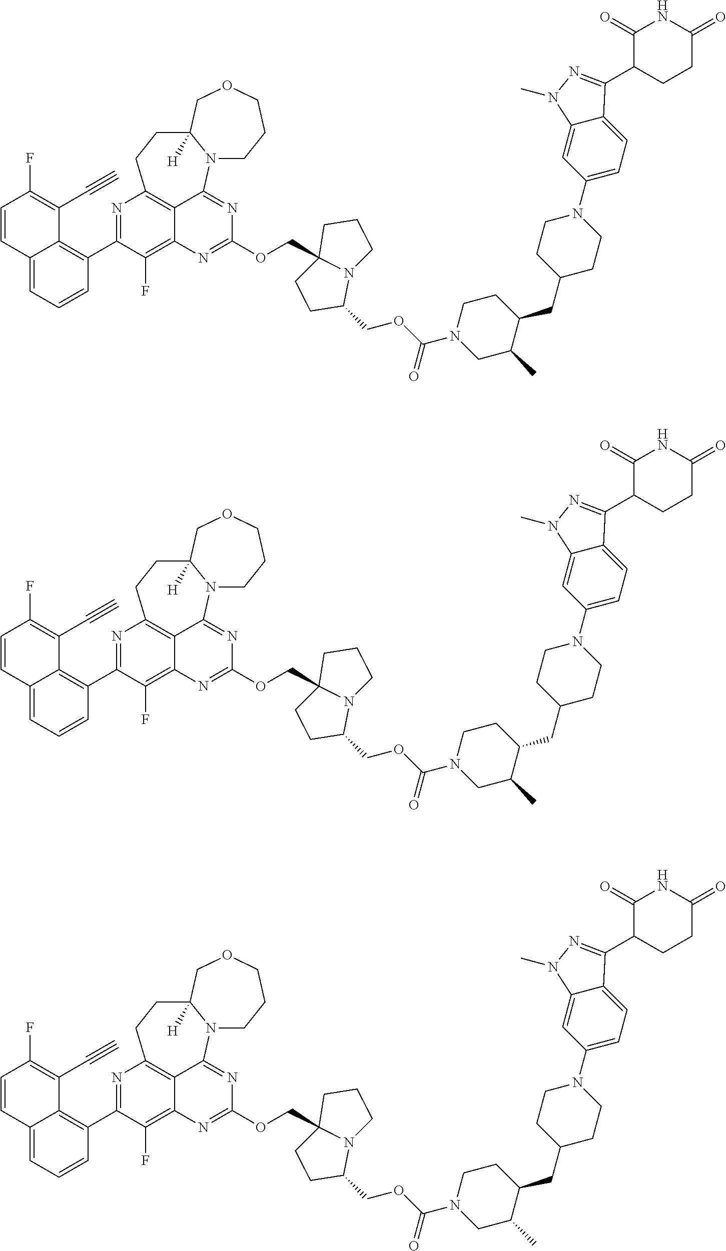

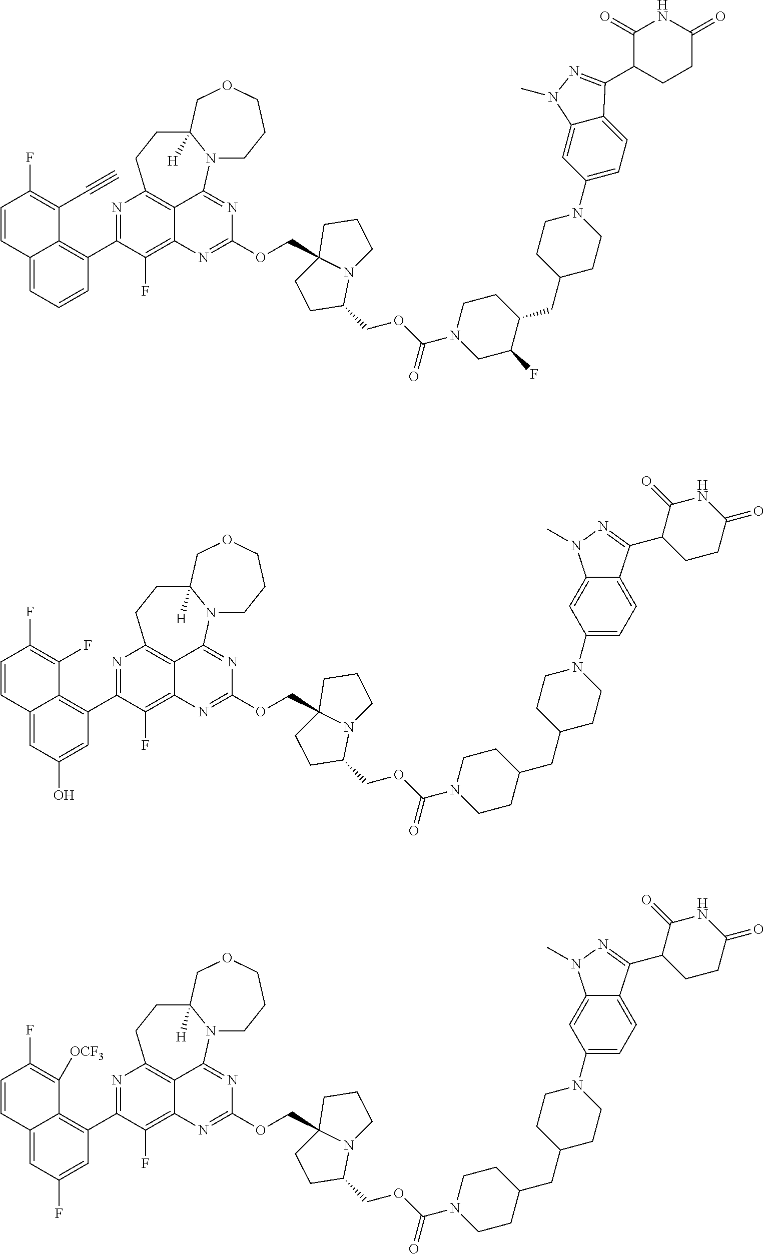

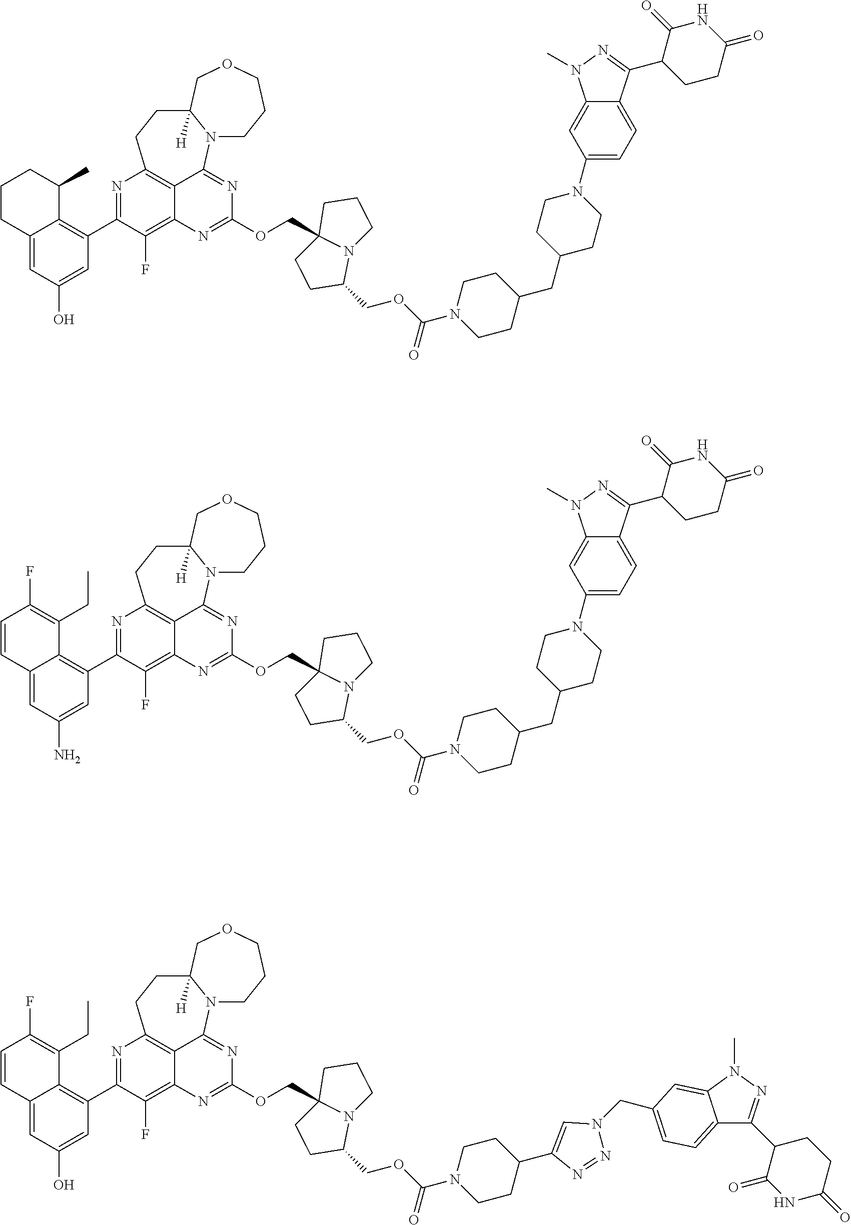

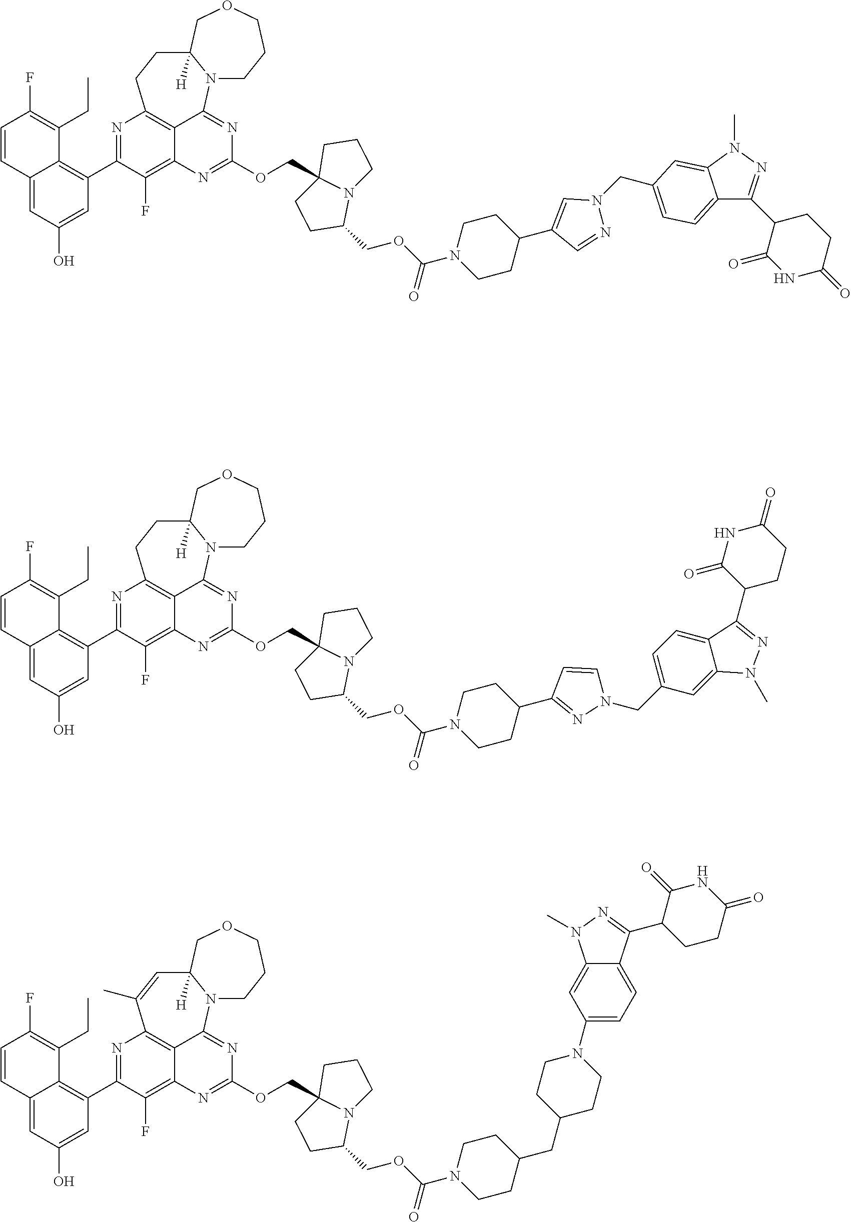

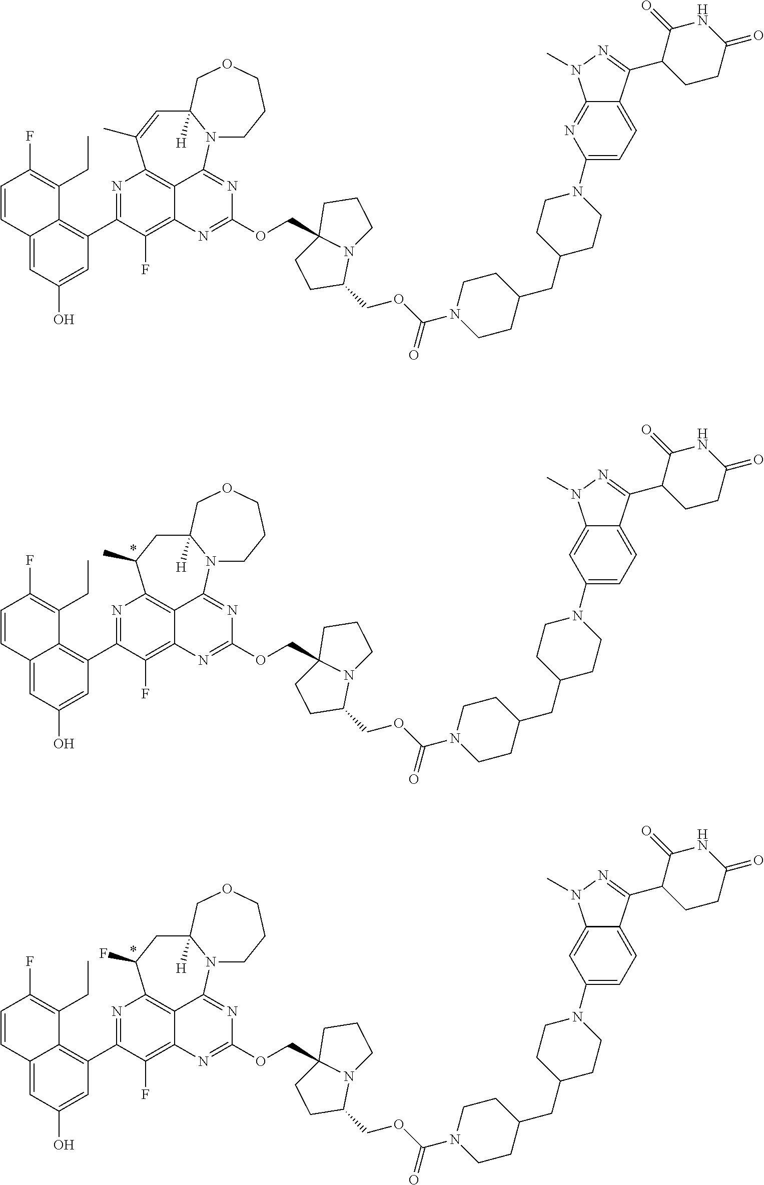

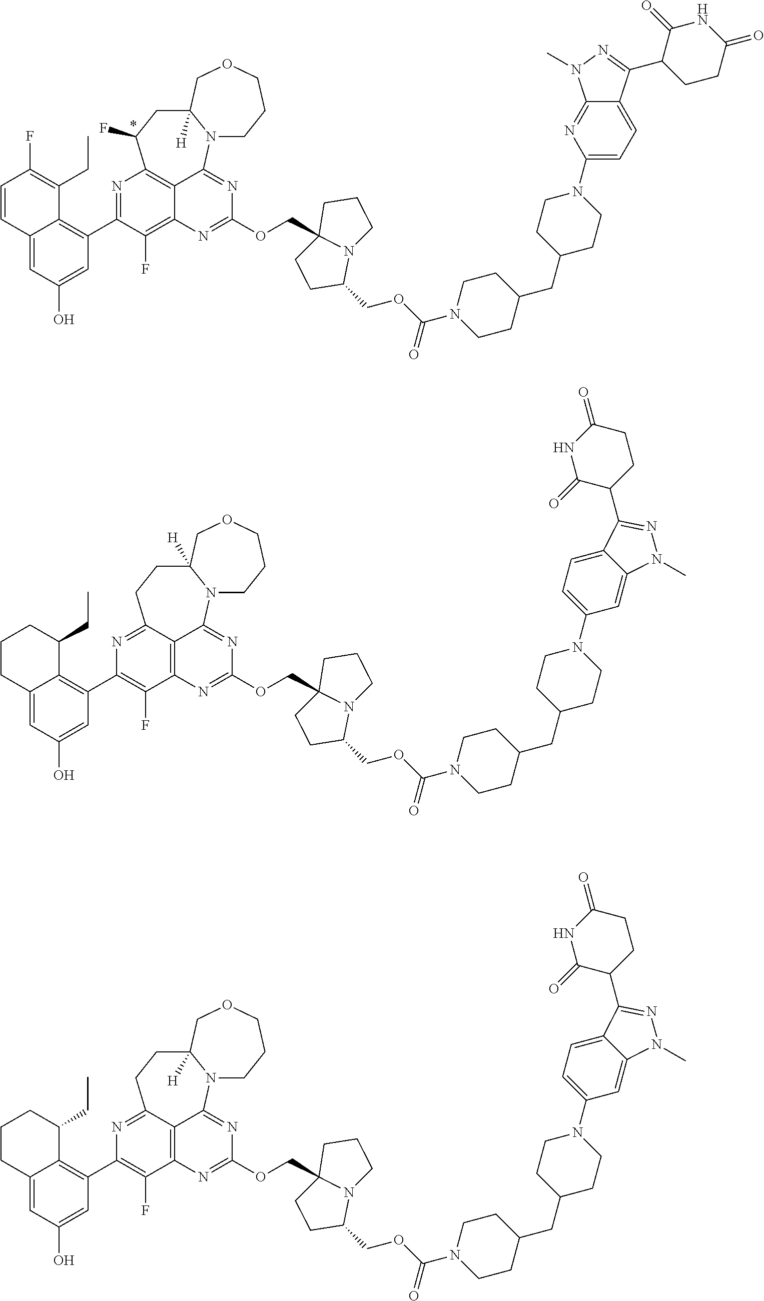

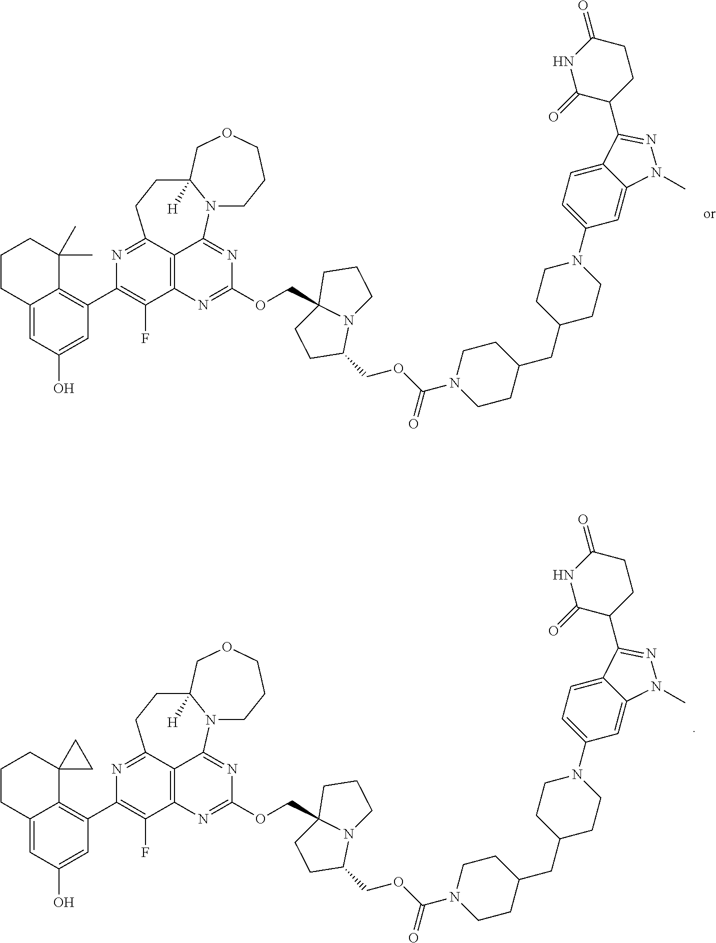

In some embodiments, the present disclosure provides the compound of Formula (I), (Ia), and/or (Ib), or a pharmaceutically acceptable salt thereof, wherein the compound is disclosed in any one of Examples 1-16, 1-19, 1-20, 1-22, 1-23, 1-25, 1-33 to 1-42, 1-44, 1-46 to 1-48, 2-10, 2-14 to 2-16, 2-20, 2-29, 2-35, 2-43 to 2-45, 2-49, 2-75, 3-1, 3-2, 4-1, 4-8, 5-1 to 5-3, 5-8 to 5-12, 6-1, and 6-3. In some embodiments, the present disclosure provides the compound of Formula (I), (Ia), and/or (Ib), or a pharmaceutically acceptable salt thereof, wherein the compound has the structure:

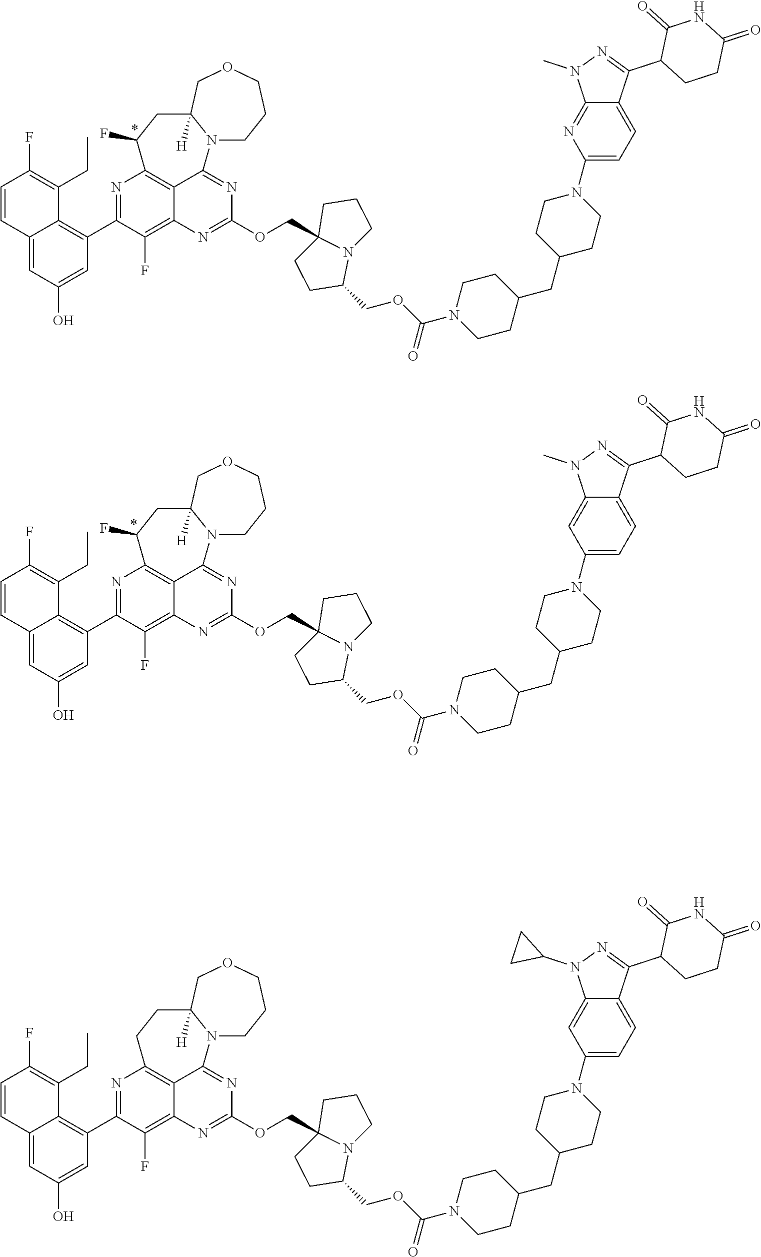

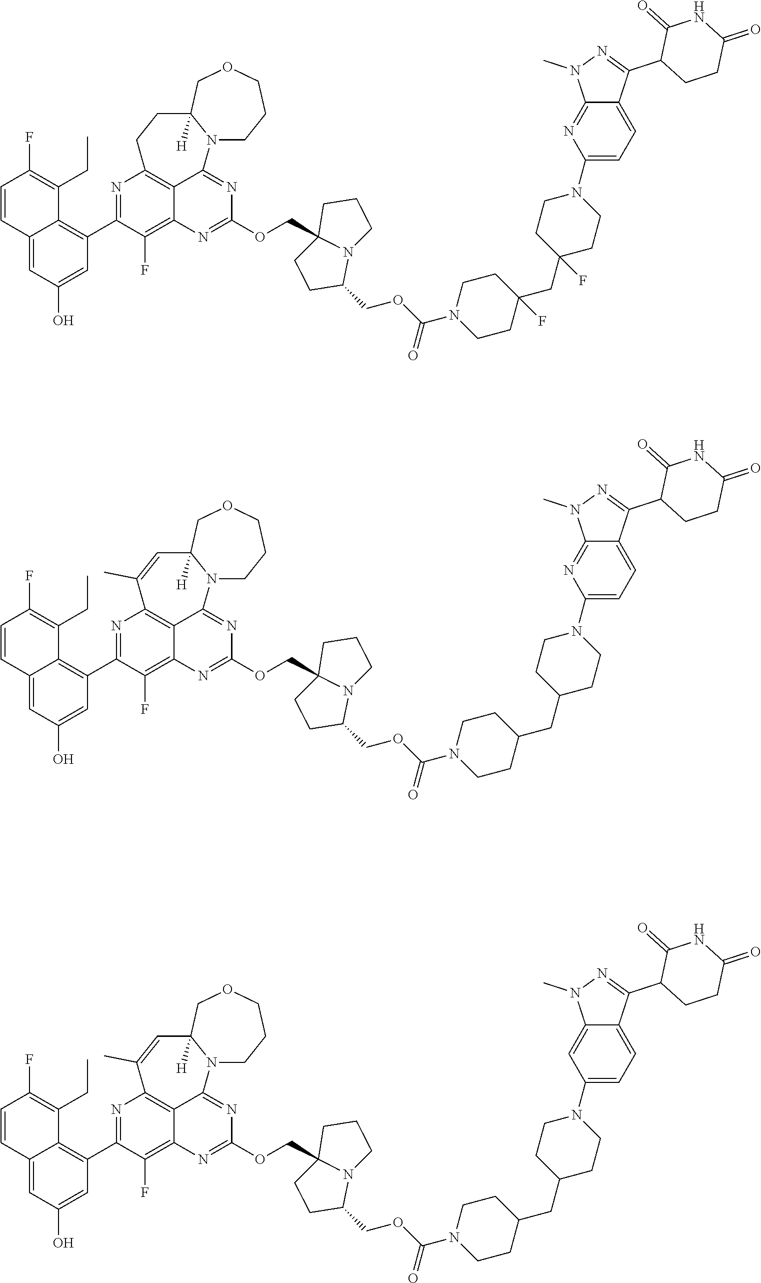

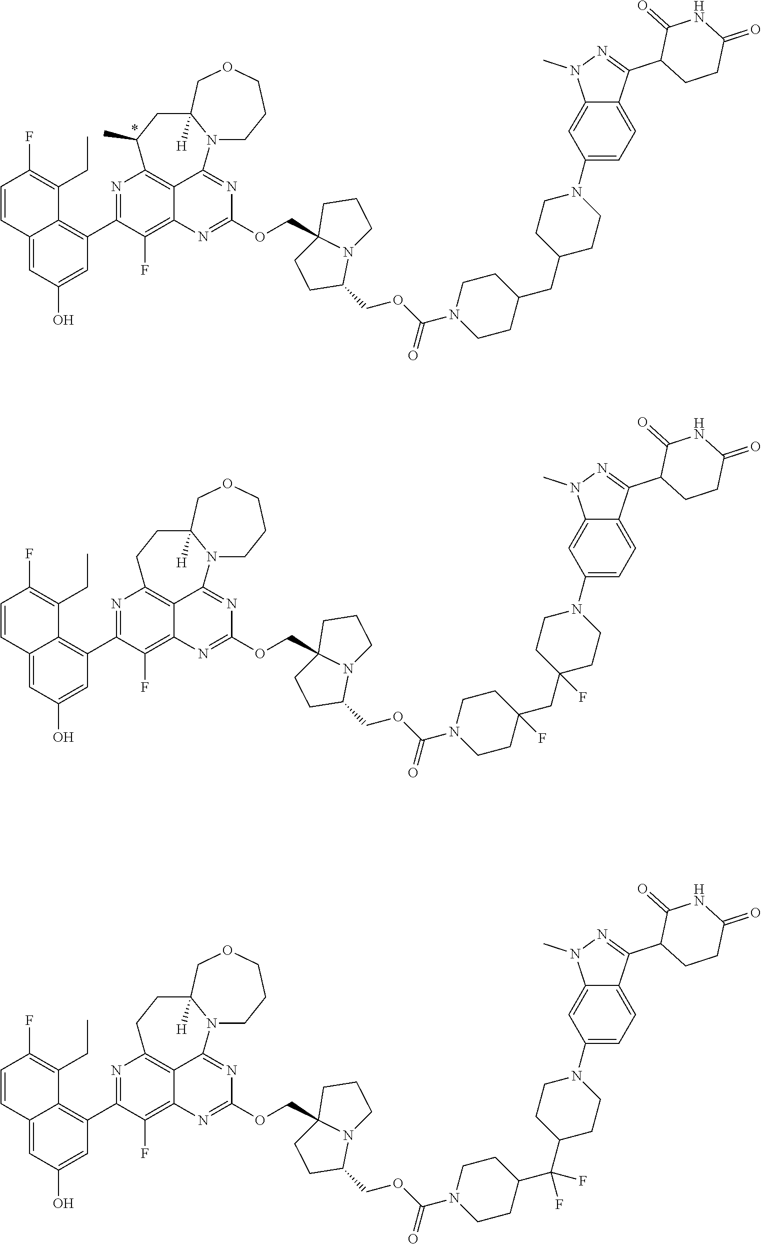

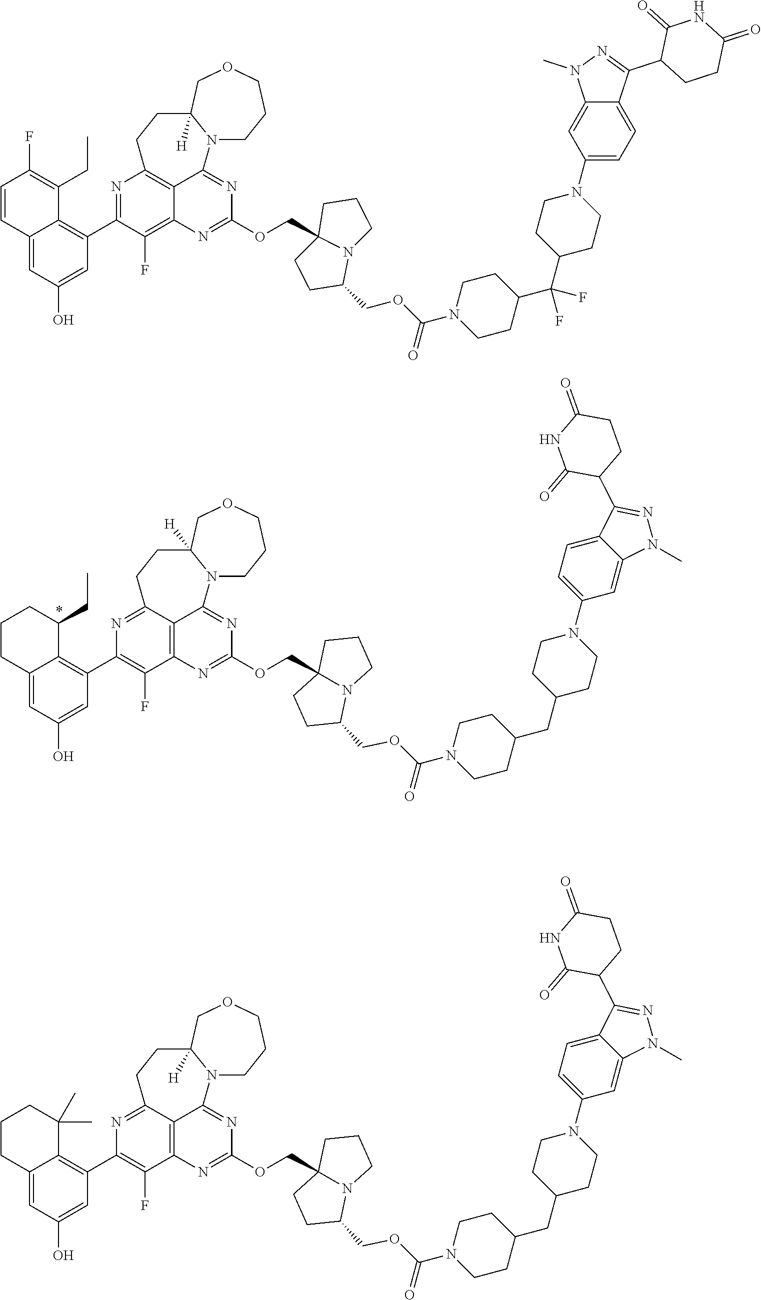

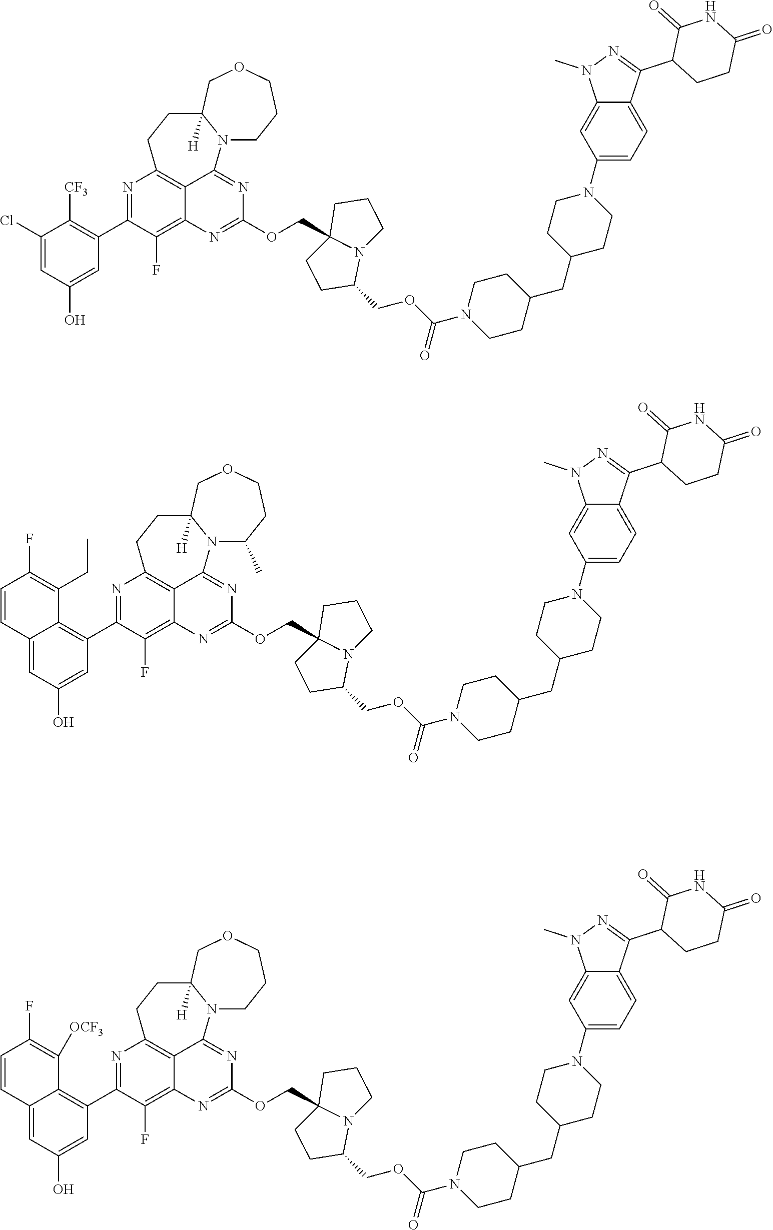

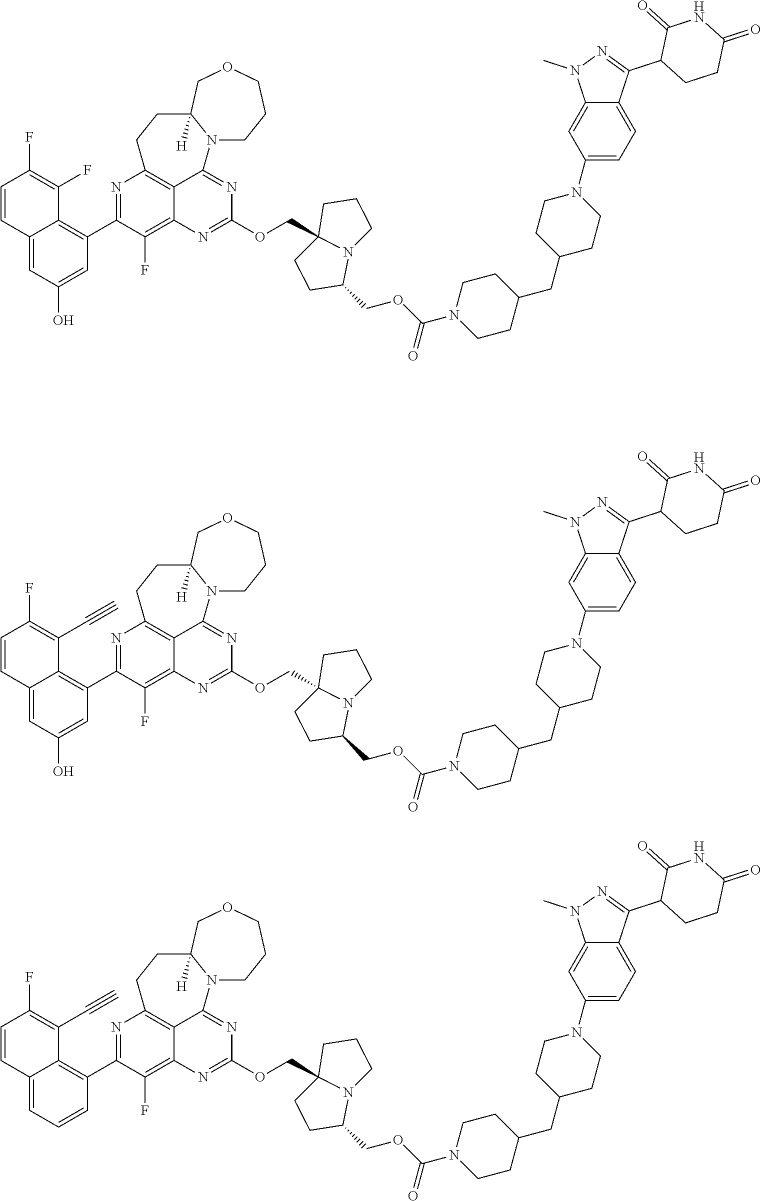

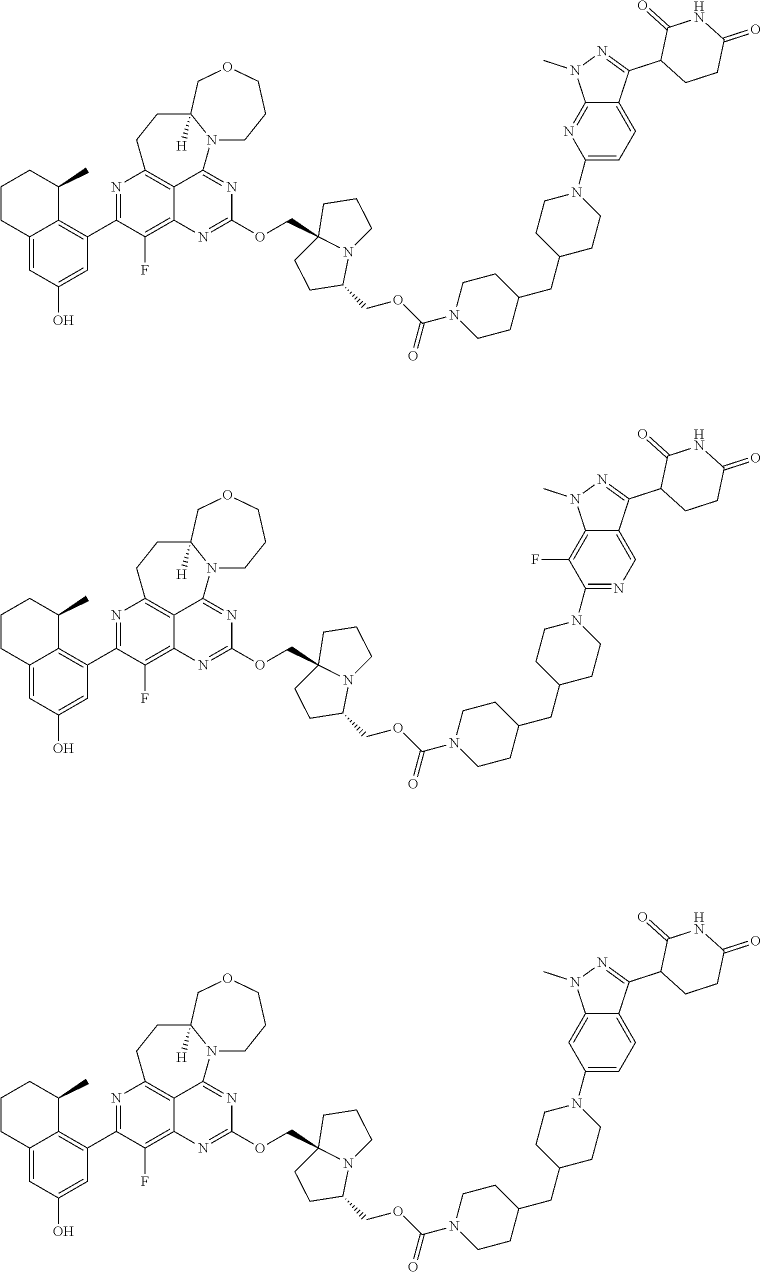

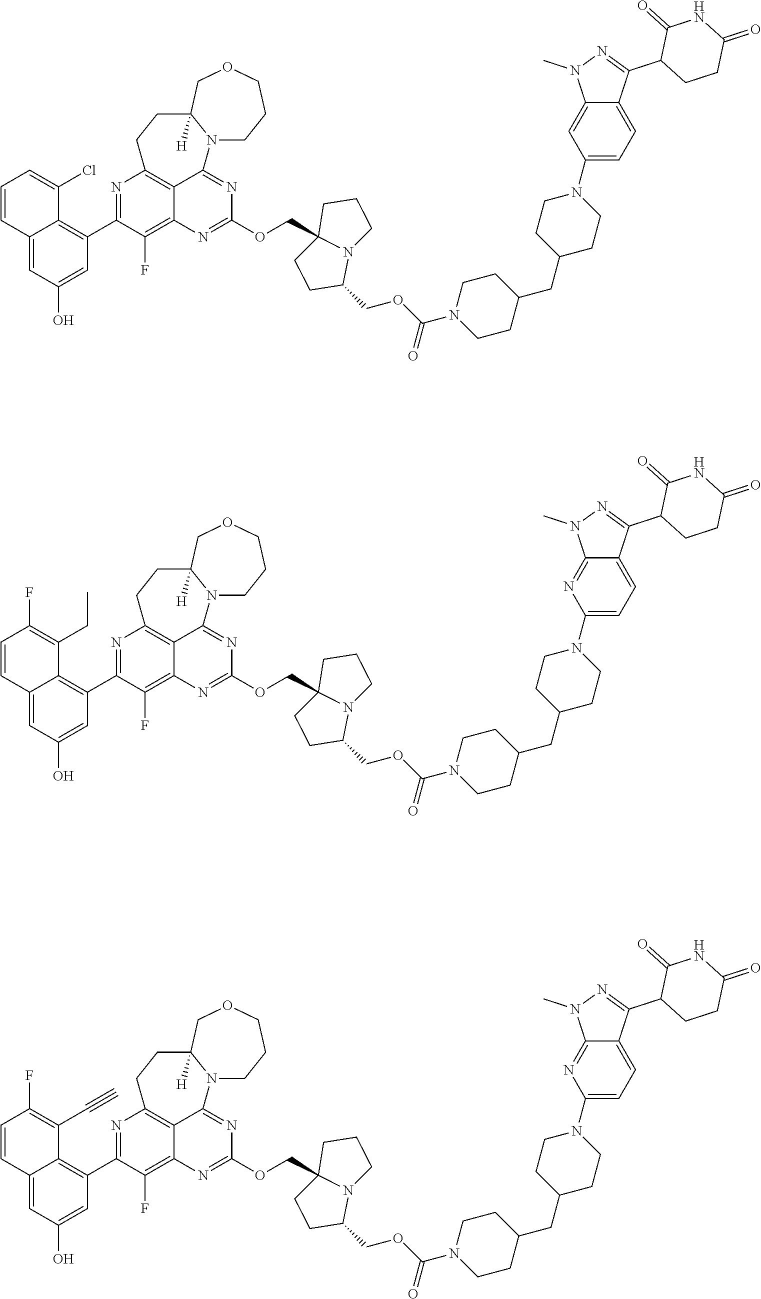

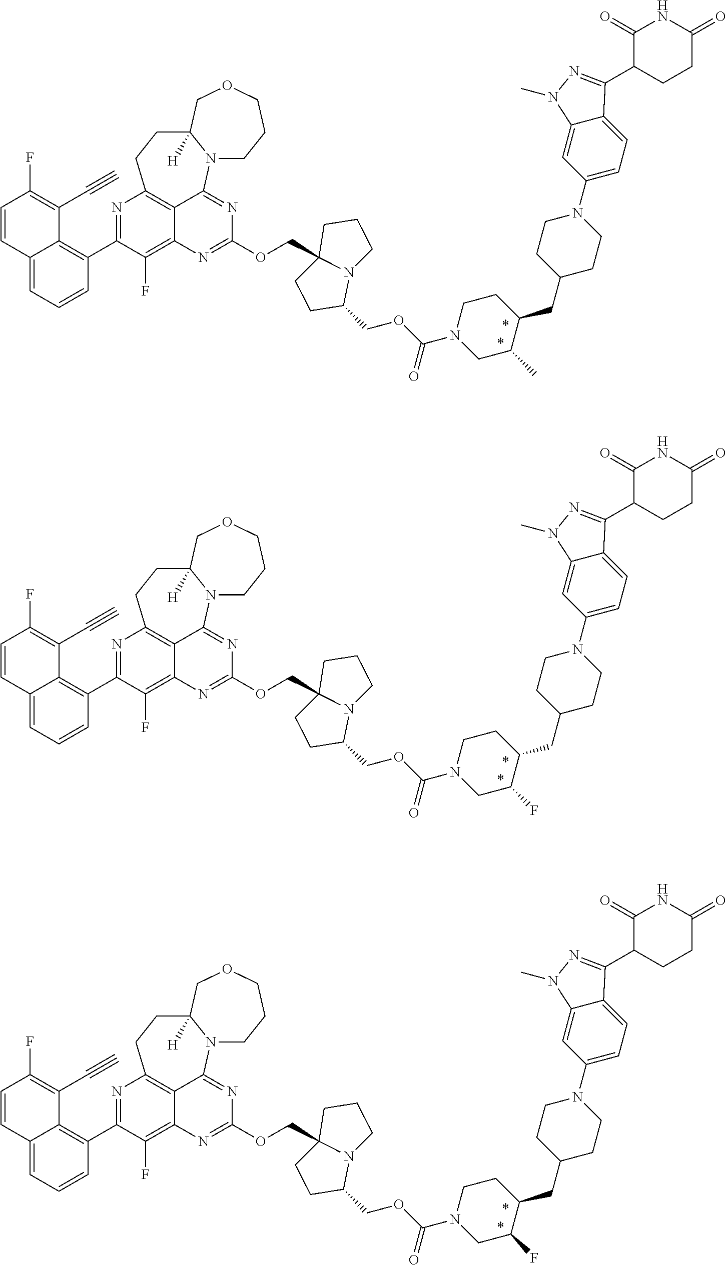

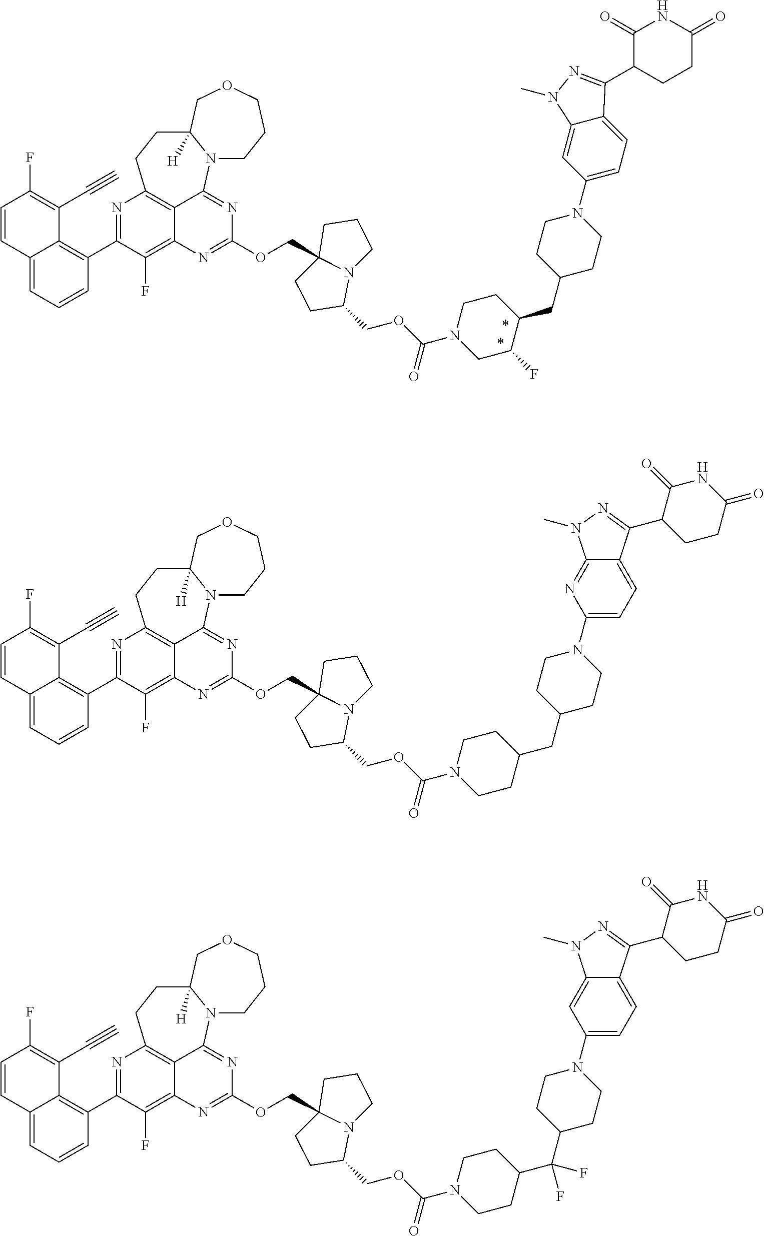

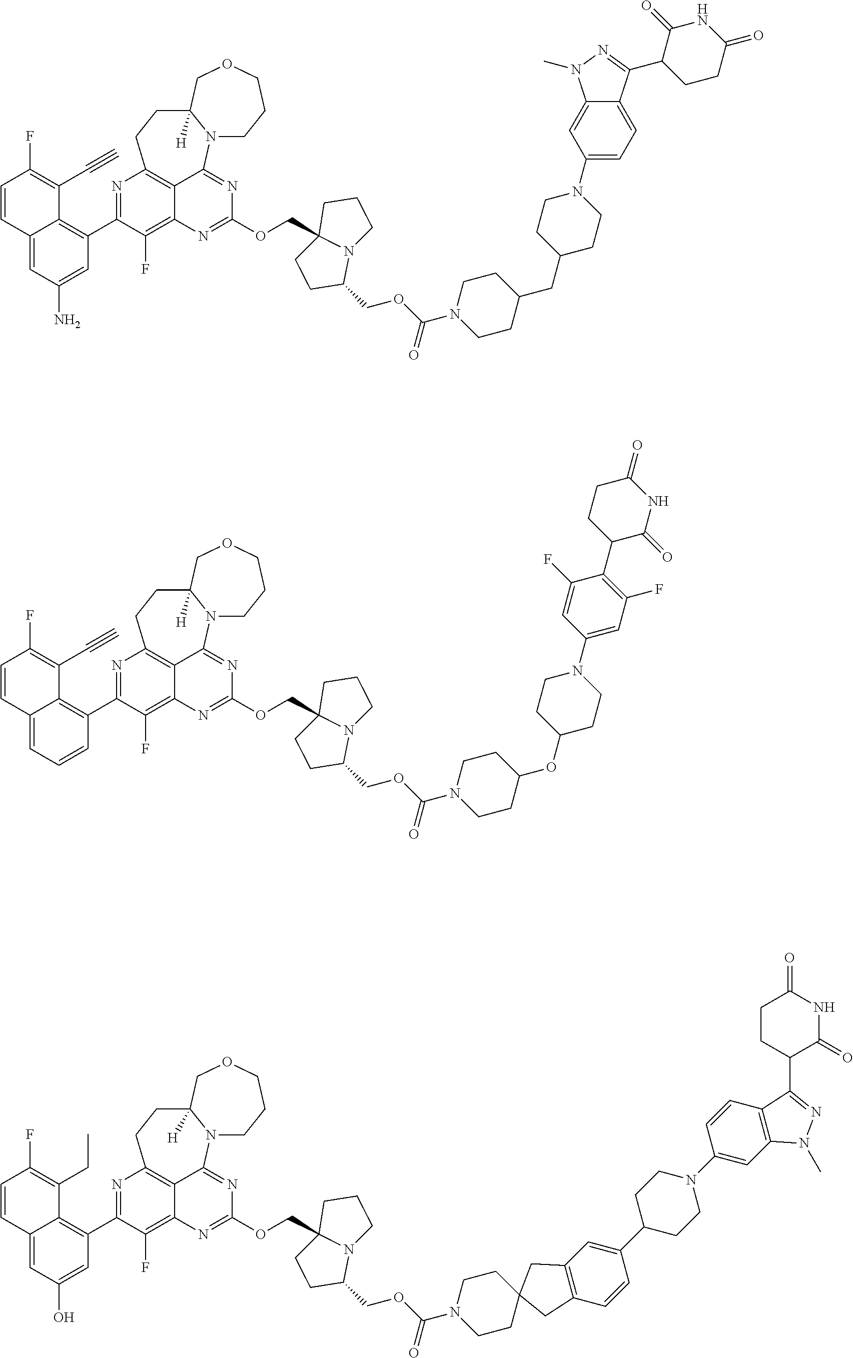

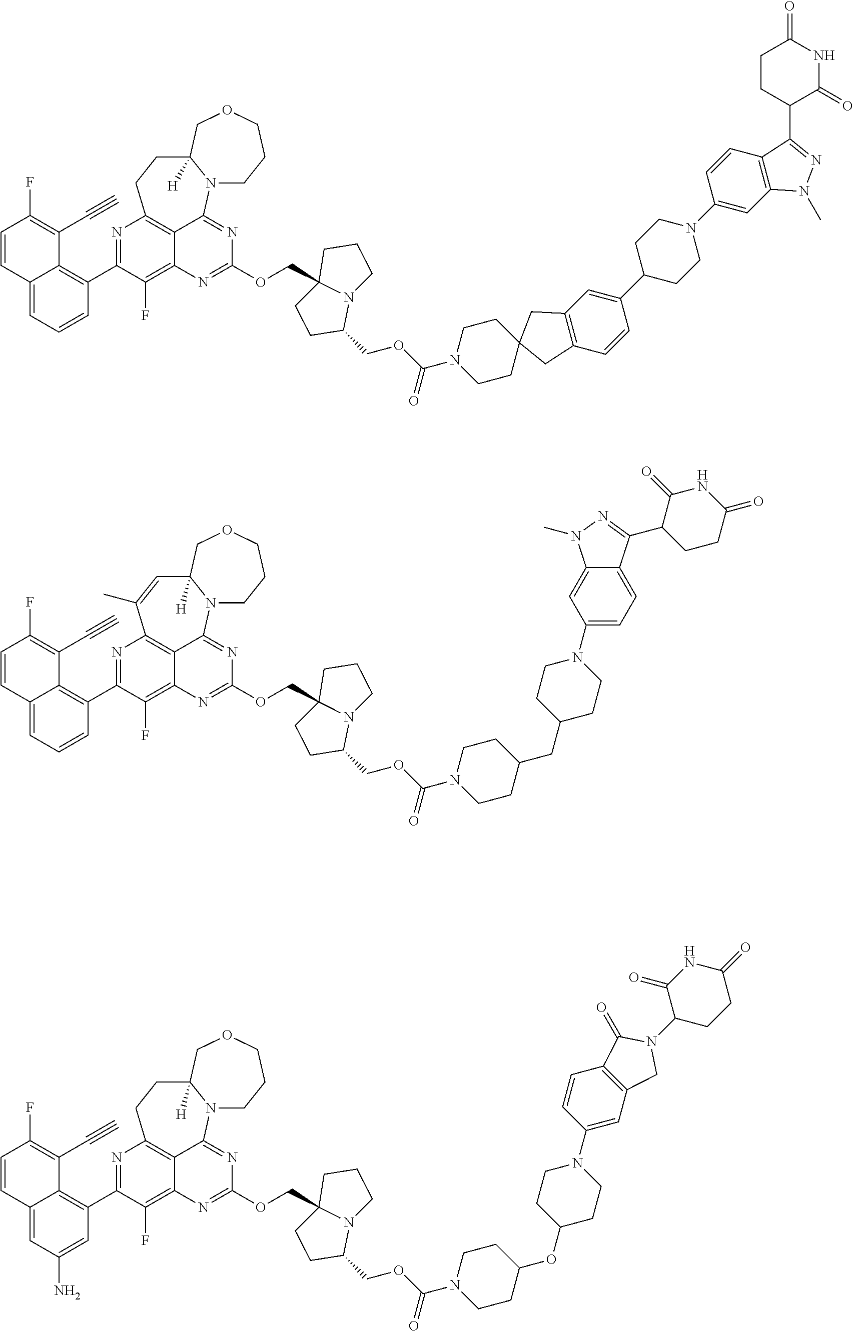

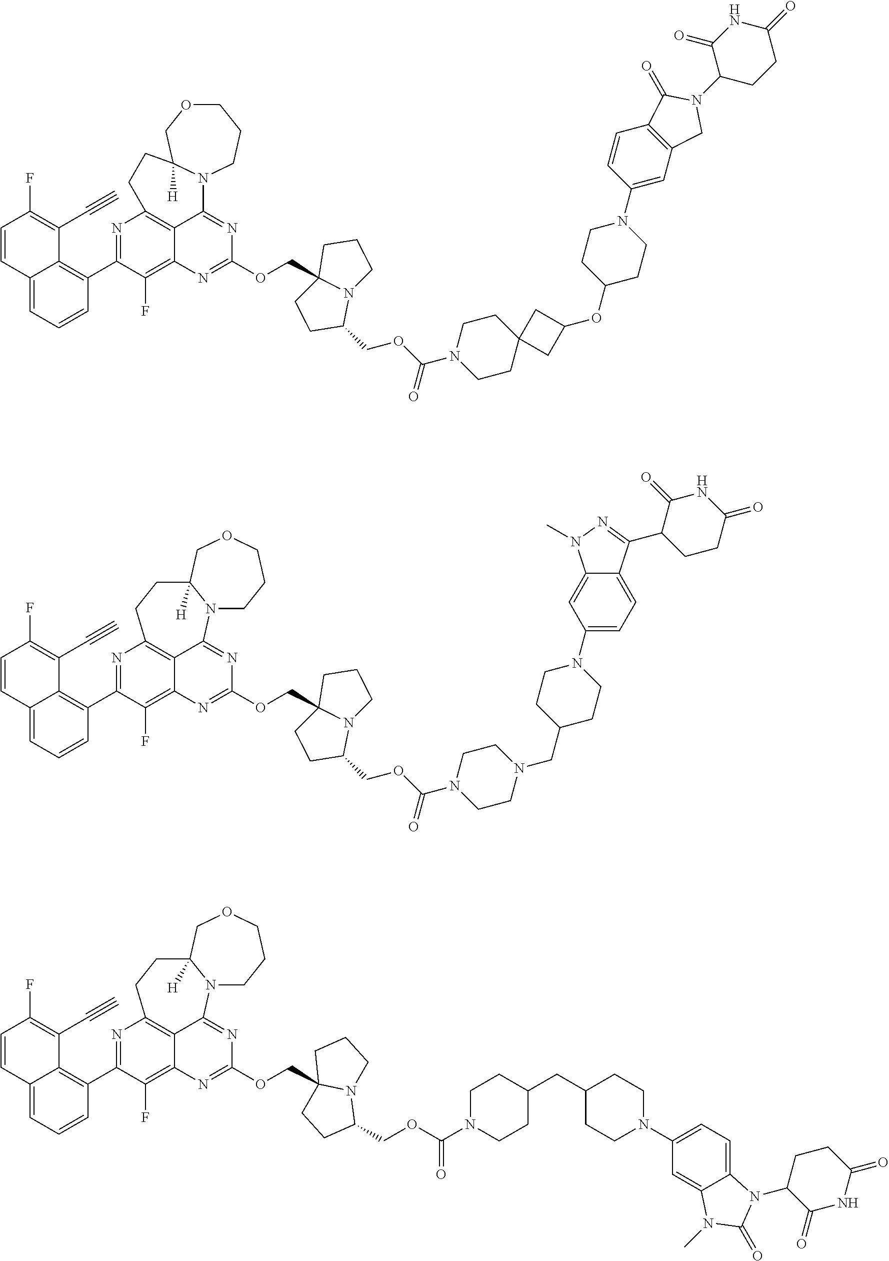

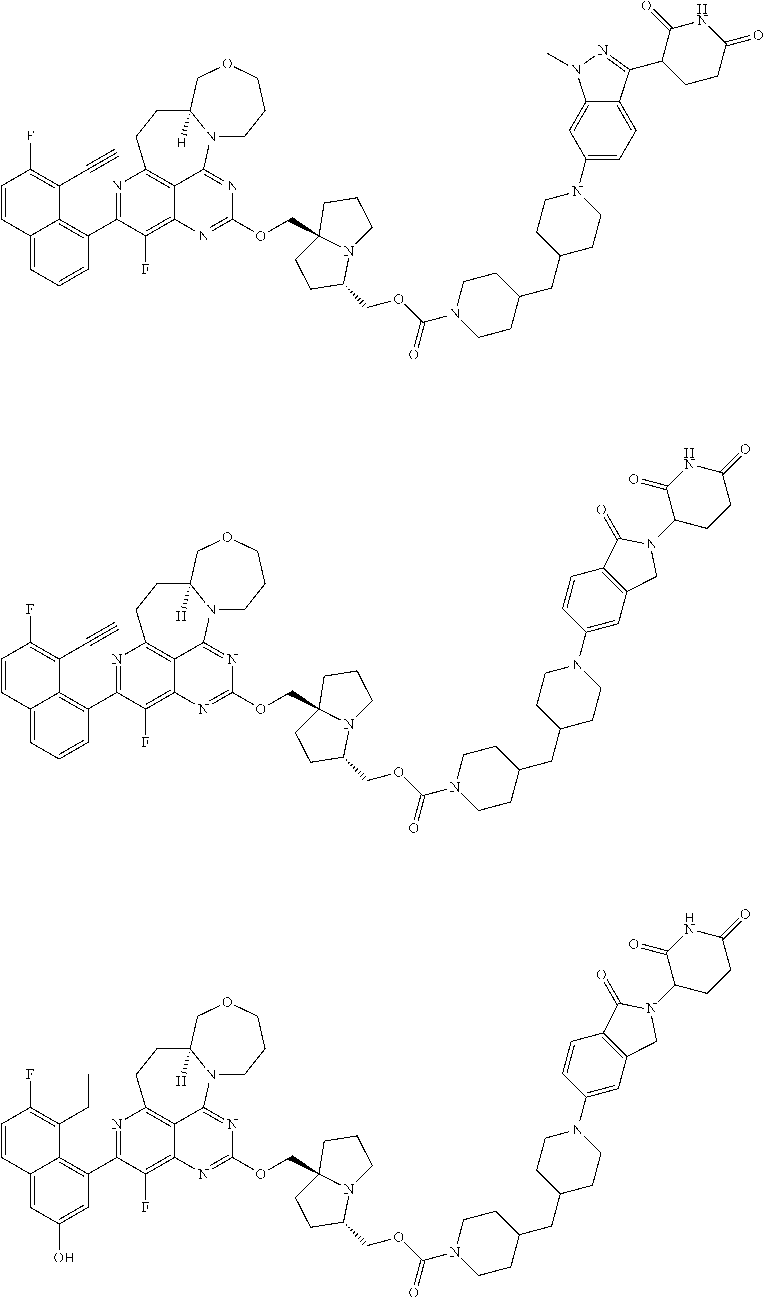

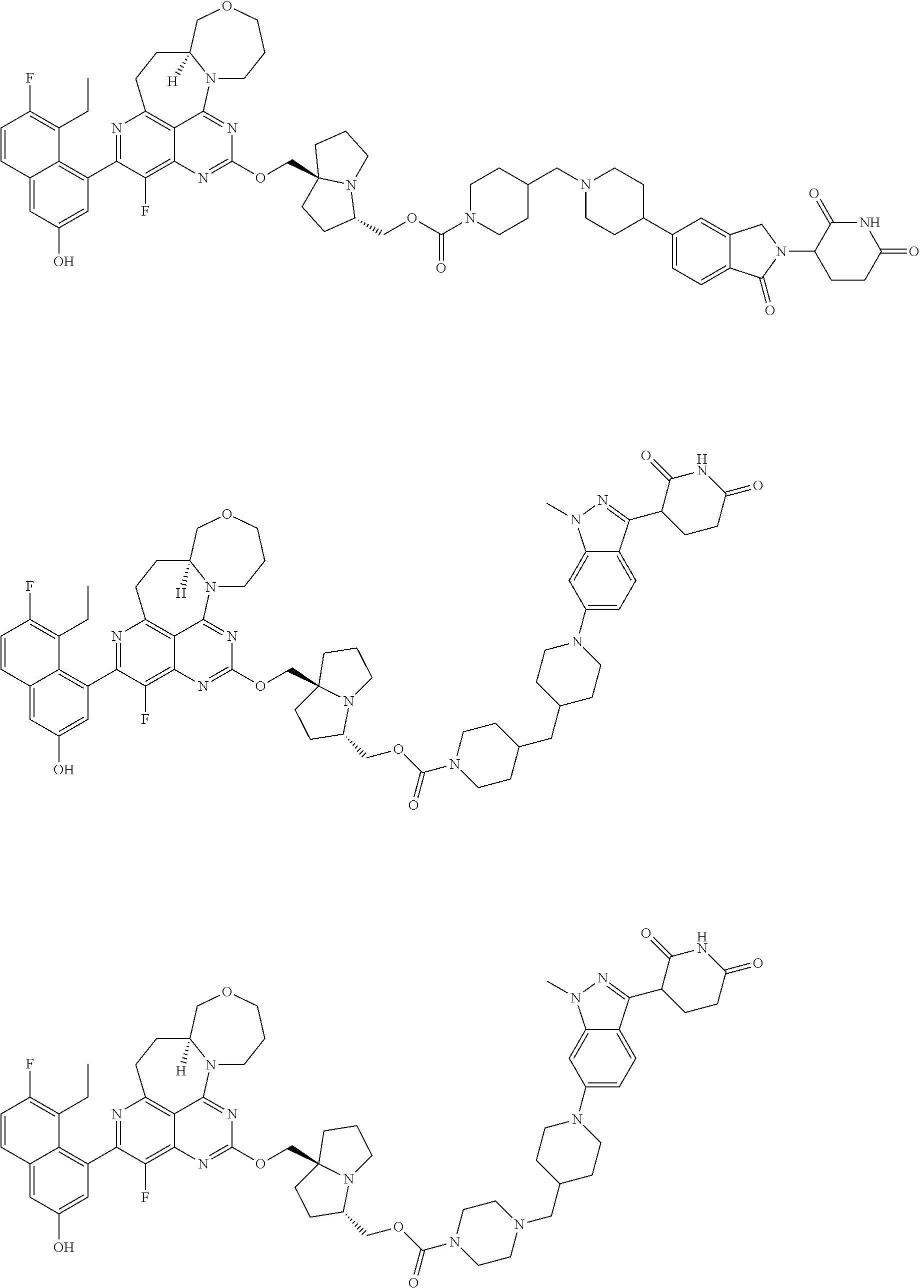

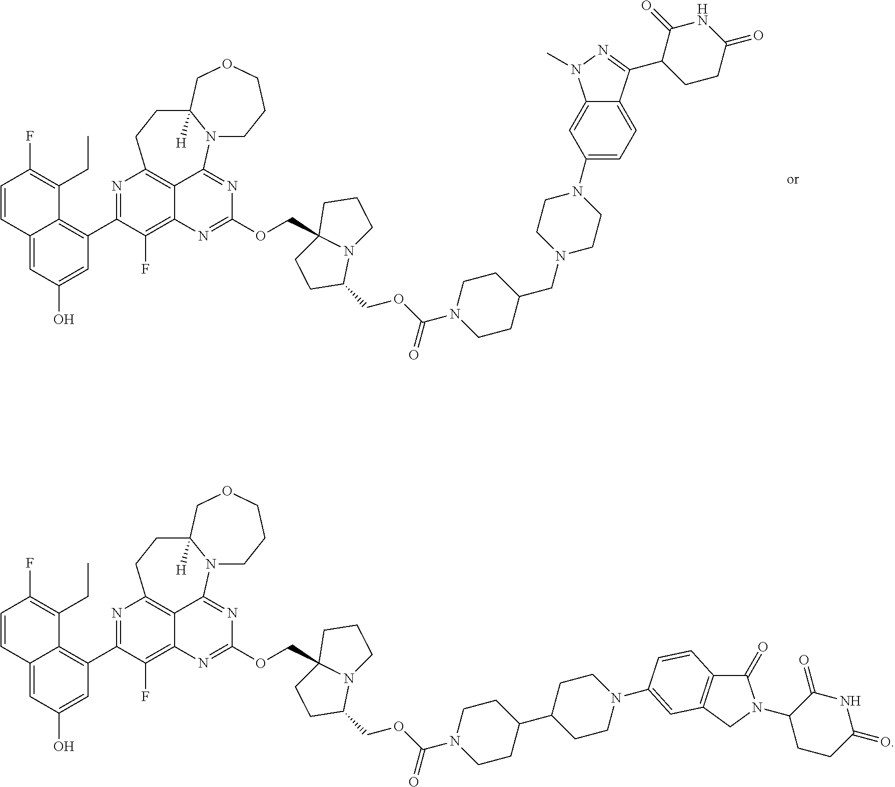

In some embodiments, the present disclosure provides the compound of Formula (I), (Ia), and/or (Ib), or a pharmaceutically acceptable salt thereof, wherein the compound has the structure:

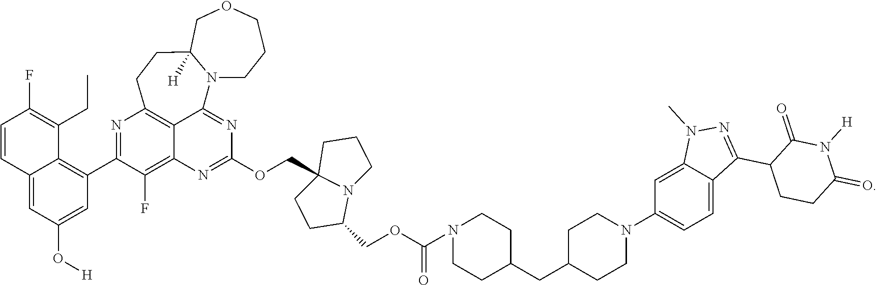

In some embodiments, the present disclosure provides the compound of Formula (I), (Ia), and/or (Ib), wherein the compound has the structure:

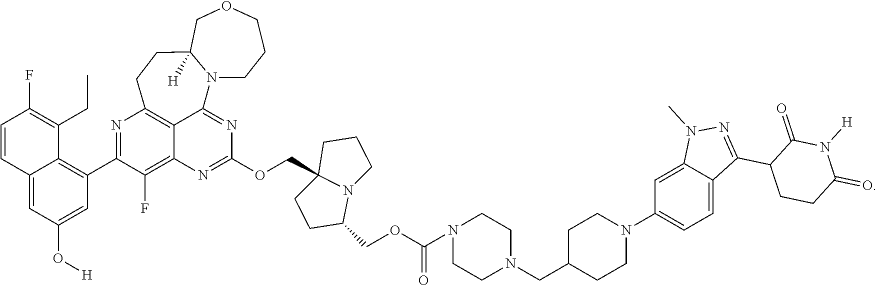

In some embodiments, the present disclosure provides the compound of Formula (I), (Ia), and/or (Ib), or a pharmaceutically acceptable salt thereof, wherein the compound has the structure:

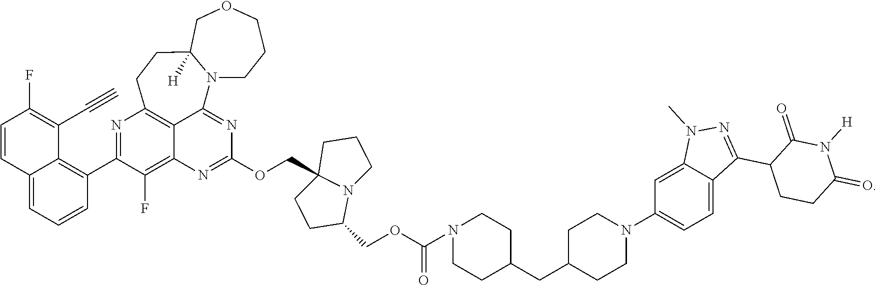

In some embodiments, the present disclosure provides the compound of Formula (I), (Ia), and/or (Ib), wherein the compound has the structure:

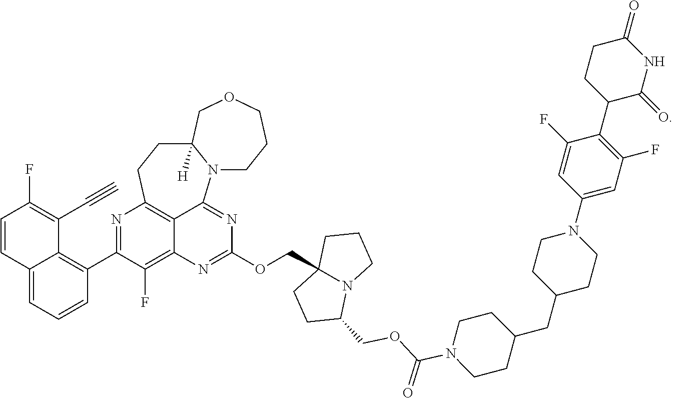

In some embodiments, the present disclosure provides the compound of Formula (I), (Ia), and/or (Ib), or a pharmaceutically acceptable salt thereof, wherein the compound has the structure:

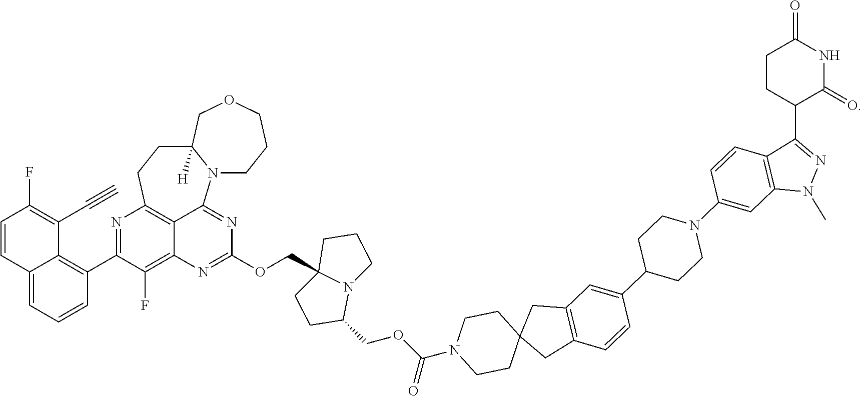

In some embodiments, the present disclosure provides the compound ofFormula (I), (Ia), and/or (Ib), wherein the compound has the structure:

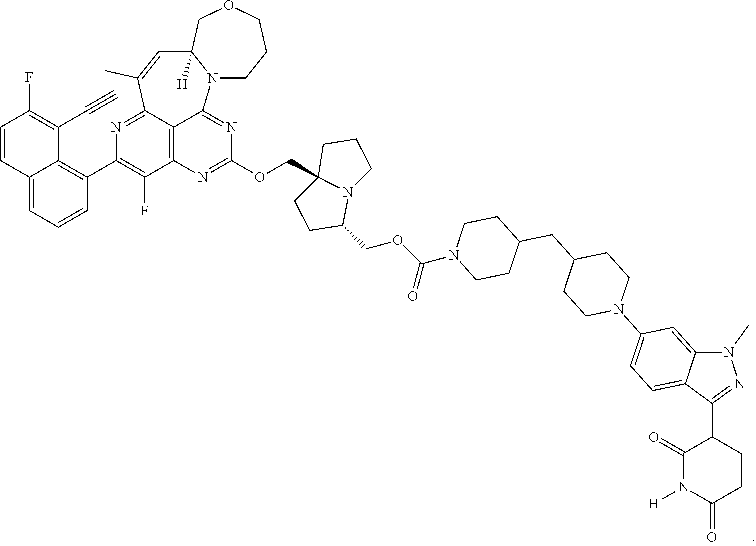

Also falling within the scope herein are the in vivo metabolic products of the compounds described herein. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no HSV antiviral activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs typically will be stable in the digestive system but may be substantially hydrolyzed to the parental drug in the digestive lumen, liver, lung or other metabolic organ, or within cells in general. As used herein, a prodrug is understood to be a compound that is chemically designed to efficiently liberate the parent drug after overcoming biological barriers to oral delivery.

### IV. Pharmaceutical Compositions

Also disclosed herein are pharmaceutical compositions comprising a pharmaceutically effective amount of a compound of the present disclosure (e.g., a compound of Formula (I), (Ia), and/or (Ib)), or a pharmaceutically acceptable salt thereof), and a pharmaceutically acceptable carrier or excipient. Also provided herein is a pharmaceutical composition comprising a pharmaceutically effective amount of a compound of the present disclosure, or a pharmaceutically acceptable salt thereof, and a pharmaceutically acceptable carrier or excipient.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986). Excipients can include, for example, ascorbic acid and other antioxidants, chelating agents such as EDTA, carbohydrates such as dextran, hydroxyalkylcellulose, hydroxyalkylmethylcellulose, stearic acid, and combinations thereof. In some embodiments, the formulation is basic. In some embodiments, the formulation is acidic. In some embodiments, the formulation has a neutral pH. In some embodiments, the pH of the formulations is from 2 to 11 (e.g., 2, 3, 4, 5, 6, 7, 8, 9, 10, 11, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, or 9-11).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with at least accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

For infections of the eye or other external tissues (e.g., mouth and skin), the formulations can be applied as a topical ointment or cream containing the active ingredient(s) in an amount of, for example, 0.075 to 20% w/w (including active ingredient(s) in a range from 0.1% to 20% in increments of 0.1% w/w such as 0.6% w/w, 0.7% w/w, etc.), from 0.2% to 15% w/w, or from 0.5% to 10% w/w. When formulated in an ointment, the active ingredients can be employed in some embodiments with either a paraffinic or a water-miscible ointment base. Alternatively, the active ingredients can be formulated in a cream with an oil-in-water cream base.

In some embodiments, the aqueous phase of the cream base can include, for example, from 30% to 90% (e.g., 35%, 40%, 45%, 50%, 55%, 60%, 65%, 70%, 75%, 80%, 85%) w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. In some embodiments, the cream base can include, for instance, a compound that enhances absorption or penetration of the active ingredient through the skin or other affected areas. Examples of such dermal penetration enhancers include, but are not limited to, dimethyl sulfoxide and related analogs. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

Emulgents and emulsion stabilizers suitable for use in the formulation can include, but are not limited to, TWEEN® 60, TWEEN® 80, SPAN® 80, cetostearyl alcohol, benzyl alcohol, myristyl alcohol, glyceryl mono-stearate, sodium lauryl sulfate, and combinations thereof.

The choice of suitable oils or fats for the formulation can be based on achieving the desired cosmetic properties. In some embodiments, the cream can be a non-greasy, non-staining, and washable product with suitable consistency to avoid leakage from tubes or other containers. In some embodiments, esters can be included, such as, for example, straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate, a blend of branched chain esters known as CRODAMOL® CAP, or a combination thereof. In some embodiments, high melting point lipids such as white soft paraffin and/or liquid paraffin or other mineral oils can be included.

In some embodiments, the compounds disclosed herein are administered alone. In some embodiments, the compounds disclosed herein are administered in pharmaceutical compositions.

In some embodiments, the pharmaceutical compositions are for veterinary use. In some embodiments, the pharmaceutical compositions are for human use. In some embodiments, the pharmaceutical compositions disclosed herein include at least one additional therapeutic agent. In some embodiments, the pharmaceutical compositions disclosed herein include one or more additional therapeutic agent. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

Pharmaceutical compositions disclosed herein can be in any form suitable for the intended method of administration. The pharmaceutical compositions disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes at least accessory ingredients. In general, the formulations can be prepared by uniformly and intimately bringing into association the active ingredient with liquid carriers or finely divided solid carriers or both, and then, if necessary, shaping the product.

When used for oral use for example, tablets, troches, lozenges, aqueous or oil suspensions, dispersible powders or granules, emulsions, hard or soft capsules, solutions, syrups or elixirs can be prepared. Formulations intended for oral use can be prepared according to any method known to the art for the manufacture of pharmaceutical compositions and such formulations can contain at least agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

Formulations for oral use can be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin, or olive oil.

Aqueous suspensions can contain the active materials in admixture with excipients suitable for the manufacture of aqueous suspensions. Such excipients can include, for instance, a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate).

The aqueous suspension can also contain, for example, at least preservatives such as ethyl or n-propyl p-hydroxy-benzoate, one or more coloring agents, one or more flavoring agents, one or more sweetening agents (such as sucrose or saccharin), or combinations thereof. Further non-limiting examples of suspending agents include cyclodextrin. In some embodiments, the suspending agent is sulfobutyl ether beta-cyclodextrin (SEB-beta-CD), for example CAPTISOL®.

Oil suspensions can be formulated by suspending the active ingredient in a vegetable oil (e.g., arachis oil, olive oil, sesame oil, coconut oil, or a combination thereof), a mineral oil such as liquid paraffin, or a combination thereof. The oral suspensions can contain, for instance, a thickening agent, such as beeswax, hard paraffin, cetyl alcohol, or a combination thereof. In some embodiments, sweetening agents, such as those set forth above, and/or flavoring agents, are added to provide a palatable oral preparation. In some embodiments, the formulations disclosed herein are preserved by the addition of an antioxidant such as ascorbic acid.

Dispersible powders and granules suitable for preparation of an aqueous suspension by the addition of water can provide the active ingredient in admixture with a dispersing or wetting agent, a suspending agent, a preservative, and combinations thereof. Suitable dispersing or wetting agents and suspending agents are exemplified by those disclosed above. Additional excipients, for example sweetening, flavoring and coloring agents, can also be present.

The pharmaceutical compositions can also be in the form of oil-in-water emulsions. The oily phase can be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion can also contain sweetening and flavoring agents. Syrups and elixirs can be formulated with sweetening agents, such as for instance, glycerol, sorbitol or sucrose. Such formulations can also contain, for instance, a demulcent, a preservative, a flavoring, a coloring agent, or a combination thereof.

The pharmaceutical compositions can be in the form of a sterile injectable or intravenous preparation, such as a sterile injectable aqueous or oleaginous suspension. This suspension can be formulated according to the known art using those suitable dispersing or wetting agents and suspending agents which have been mentioned above. The sterile injectable or intravenous preparation can also be a sterile injectable solution or suspension in a non-toxic parenterally acceptable diluent or solvent, such as a solution in 1,3-butane-diol or prepared as a lyophilized powder. Among the acceptable vehicles and solvents that can be employed are water, Ringer's solution and isotonic sodium chloride solution. In addition, sterile fixed oils can be employed as a solvent or suspending medium. For this purpose, any bland fixed oil can be employed including synthetic mono- or diglycerides. In addition, fatty acids such as oleic acid can likewise be used in the preparation of injectables. Among the acceptable vehicles and solvents that can be employed include, but are not limited to, water, Ringer's solution isotonic sodium chloride solution, and hypertonic sodium chloride solution.

The amount of active ingredient that can be combined with the carrier material to produce a single dosage form will vary depending upon the host treated and the particular mode of administration. For example, a time-release formulation intended for oral administration to humans can contain approximately 1 mg to 2000 mg of active material compounded with an appropriate and convenient amount of carrier material, which can vary from 5% to 95% of the total formulations (weight:weight). For example, a time-release formulation intended for oral administration to humans can contain approximately 1 mg to 1000 mg of active material compounded with an appropriate and convenient amount of carrier material, which can vary from 5% to 95% of the total formulations (weight:weight). The pharmaceutical composition can be prepared to provide easily measurable amounts for administration. For example, an aqueous solution intended for intravenous infusion can contain from 3 μg to 500 μg of the active ingredient per milliliter of solution in order that infusion of a suitable volume at a rate of 30 mL/hr can occur.

Formulations suitable for topical administration to the eye also include eye drops wherein the active ingredient is dissolved or suspended in a suitable carrier, especially an aqueous solvent for the active ingredient. In some embodiments, the compounds disclosed herein are included in the pharmaceutical compositions disclosed herein in a concentration of 0.5% to 20% (e.g., 0.5% to 10%, 1.5% w/w).

Formulations suitable for topical administration in the mouth include lozenges can comprise an active ingredient (i.e., a compound disclosed herein and/or additional therapeutic agents) in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier, for example water for injection, immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

It should be understood that in addition to the ingredients particularly mentioned above the formulations can include other agents conventional in the art having regard to the type of formulation in question, for example those suitable for oral administration can include flavoring agents.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical compositions containing as active ingredient one or more of the compounds (“controlled release formulations”) in which the release of the active ingredient can be controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical composition, and will be determined by the clinician using conventional dose escalation studies. In some embodiments, the effective dose is from 0.0001 to 100 mg/kg body weight per day; for instance, from 10 to 30 mg/kg body weight per day; from 15 to 25 mg/kg body weight per day; from 10 to 15 mg/kg body weight per day; or from 20 to 30 mg/kg body weight per day. For example, the daily candidate dose for an adult human of approximately 70 kg body weight can range from 1 mg to 2000 mg (e.g., from 5 mg to 500 mg, from 500 mg to 1000 mg, from 1000 mg to 1500 mg, from 1500 mg to 2000 mg), and can take the form of single or multiple doses. For example, the daily candidate dose for an adult human of approximately 70 kg body weight can range from 1 mg to 1000 mg (e.g., from 5 mg to 500 mg), and can take the form of single or multiple doses.

### V. Kits

Also provided herein are kits that includes a compound disclosed herein or a pharmaceutically acceptable salt thereof. In some embodiments the kits described herein can comprise a label and/or instructions for use of the compound in the treatment of a disease or condition in a subject (e.g., human) in need thereof. In some embodiments, the disease or condition is viral infection.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof; and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more of the compounds of Formula (I), (Ia), and/or (Ib), or a pharmaceutically acceptable salt thereof (herein referred to as the active ingredients), are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the route may vary with for example the condition of the recipient. An advantage of the compounds herein is that they are orally bioavailable and can be dosed orally.

The compounds of the present disclosure (also referred to herein as the active ingredients), can be administered by any route appropriate to the condition to be treated.

Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the route may vary with for example the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The dosage or dosing frequency of a compound of the present disclosure may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A compound of the present disclosure may be combined with one or more additional therapeutic agents in any dosage amount of the compound of the present disclosure (e.g., from about 1 mg to about 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the compound of the present disclosure are about 100, about 125, about 150, about 175, about 200, about 225, about 250, about 275, about 300, about 325, about 350, about 375, about 400, about 425, about 450, about 475, or about 500 mg per dose. Other therapeutically effective amounts of the compound of the present disclosure are about 100 mg per dose, or about 125, about 150, about 175, about 200, about 225, about 250, about 275, about 300, about 325, about 350, about 375, about 400, about 425, about 450, or about 500 mg per dose. A single dose can be administered hourly, daily, or weekly. For example, a single dose can be administered once about every 1 hour, about 2, about 3, about 4, about 6, about 8, about 12, about 16 or once about every 24 hours. A single dose can also be administered once about every 1 day, about 2, about 3, about 4, about 5, about 6, or once about every 7 days. A single dose can also be administered once about every 1 week, about 2, about 3, or once about every 4 weeks. In some embodiments, a single dose can be administered once about every week. A single dose can also be administered once about every month.

Other therapeutically effective amounts of the compound of the present disclosure are about 20, about 25, about 30, about 35, about 40, about 45, about 50, about 55, about 60, about 65, about 70, about 75, about 80, about 85, about 90, about 95, or about 100 mg per dose.

The frequency of dosage of the compound of the present disclosure can be determined by the needs of the individual patient and can be, for example, once per day or twice, or more times, per day. Administration of the compound continues for as long as necessary to treat the disease or condition. For example, a compound can be administered to a human having cancer for a period of from about 20 days to about 180 days or, for example, for a period of from about 20 days to about 90 days or, for example, for a period of from about 30 days to about 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from about 1 to about 14 days, followed by a period of about 7 to about 21 days during which the patient does not receive a dose of the compound, followed by a subsequent period (e.g., from about 1 to about 14 days) during which the patient again receives a daily dose of the compound. Alternating periods of administration of the compound, followed by non-administration of the compound, can be repeated as clinically required to treat the patient.

In some embodiments, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In some embodiments, kits comprising a compound of the present disclosure, or a pharmaceutically acceptable salt thereof, in combination with one or more (e.g., one, two, three, four, one or two, one to three, or one to four) additional therapeutic agents are provided.

In some embodiments, a compound of the present disclosure, or a pharmaceutically acceptable salt thereof, is combined with one, two, three, four or more additional therapeutic agents. In some embodiments, a compound of the present disclosure, or a pharmaceutically acceptable salt thereof, is combined with two additional therapeutic agents. In some embodiments, a compound of the present disclosure, or a pharmaceutically acceptable salt thereof, is combined with three additional therapeutic agents. In some embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of the present disclosure is combined with one or more additional therapeutic agents as described herein, the components of the composition are administered as a simultaneous or sequential regimen. When administered sequentially, the combination may be administered in two or more administrations.

In some embodiments, a compound of the present disclosure is combined with one or more additional therapeutic agents in a unitary dosage form for simultaneous administration to a patient, for example as a solid dosage form for oral administration.

In some embodiments, a compound of the present disclosure is co-administered with one or more additional therapeutic agents.

In order to prolong the effect of a compound of the present disclosure, it is often desirable to slow the absorption of a compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending a compound in an oil vehicle. Injectable depot forms are made by forming microencapsule matrices of a compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping a compound in liposomes or microemulsions that are compatible with body tissues.

### VII. Methods of Use

The disclosure further relates to the use of compounds disclosed herein for the treatment and/or prophylaxis of diseases and/or conditions through inhibition of KRAS wild type, G12C, G12D and/or G12V. Further, the present disclosure relates to the use of said compounds for the preparation of a medicament for the treatment and/or prophylaxis of cancer.

The disclosure further relates to the use of compounds disclosed herein for the treatment and/or prophylaxis of diseases and/or conditions through degradation of KRAS wild type, G12C, G12D and/or G12V. In some embodiments, the compounds disclosed herein are degraders of KRAS wild type, G12C, G12D and/or G12V.

Medicaments as referred to herein can be prepared by conventional processes, including the combination of a compound according to the present disclosure and a pharmaceutically acceptable carrier.

In some embodiments, provided herein is a method of inhibiting KRAS wild type, G12C, G12D and/or G12V protein in a subject in need thereof, the method comprising administering to the subject a therapeutically effective amount of a compound of Formula (I), (Ia), and/or (Ib), or a pharmaceutically acceptable salt thereof, or a pharmaceutical composition comprising a compound of Formula (I), (Ia), and/or (Ib).

In some embodiments, provided herein is a method of degrading KRAS wild type, G12C, G12D and/or G12V protein in a subject in need thereof, the method comprising administering to the subject a therapeutically effective amount of a compound of Formula (I), (Ia), and/or (Ib), or a pharmaceutically acceptable salt thereof, or a pharmaceutical composition comprising a compound of Formula (I), (Ia), and/or (Ib).

In some embodiments, provided herein is treating cancer in a subject in need thereof, the method comprising administering to the subject a therapeutically effective amount of a compound of Formula (I), (Ia), and/or (Ib), or a pharmaceutically acceptable salt thereof, or a pharmaceutical composition comprising a compound of Formula (I), (Ia), and/or (Ib).

In some embodiments, provided herein is a method of treating and/or preventing a cancer.

In some embodiments, provided herein is a method of treating and/or preventing a KRAS G12D-associated cancer.

In some embodiments, provided herein is a method of reducing the proliferation of a cell comprising contacting the cell with a compound of Formula (I), (Ia), and/or (Ib), or a pharmaceutically acceptable salt thereof.

In some embodiments, the KRAS G12C, G12D and/or G12V associated disease or condition includes cancer. In some embodiments, the cancer is a hematological cancer. In some embodiments, the cancer includes a solid tumor. In some embodiments, the cancer includes a malignant tumor. In some embodiments the cancer includes a metastatic cancer. In some embodiments, the cancer is resistant or refractory to one or more anticancer therapies. In some embodiments, greater than about 50% of the cancer cells detectably express one or more cell surface immune checkpoint receptors (e.g., so-called “hot” cancer or tumor). In some embodiments, greater than about 1% and less than about 50% of the cancer cells detectably express one or more cell surface immune checkpoint receptors (e.g., so called “warm” cancer or tumor). In some embodiments, less than about 1% of the cancer cells detectably express one or more cell surface immune checkpoint receptors (e.g., so called “cold” cancer or tumor).

In some embodiments, the KRAS G12C, G12D and/or G12V associated disease or condition is a hematological cancer, e.g., a leukemia (e.g., Acute Myelogenous Leukemia (AML), Acute Lymphoblastic Leukemia (ALL), B-cell ALL, Myelodysplastic Syndrome (MDS), myeloproliferative disease (MPD), Chronic Myelogenous Leukemia (CML), Chronic Lymphocytic Leukemia (CLL), undifferentiated leukemia), a lymphoma (e.g., small lymphocytic lymphoma (SLL), mantle cell lymphoma (MCL), follicular lymphoma (FL), T-cell lymphoma, B-cell lymphoma, diffuse large B-cell lymphoma (DLBCL), marginal zone lymphoma (MZL), Waldestrom's macroglobulinemia (WM)) and/or a myeloma (e.g., multiple myeloma (MM)).

In some embodiments, the KRAS G12C, G12D and/or G12V associated disease or condition is an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), or a lymphoma.

In some embodiments, the KRAS G12C, G12D and/or G12V associated disease or condition includes a solid tumor in or arising from a tissue or organ, such as:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumor of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g. carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma; hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma;
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, Müllerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma—MMMT)), endometrial stromal tumors, endometrial malignant mullerian mixed tumors, gestational trophoblastic tumors (partial hydatiform mole, complete hydatiform mole, invasive hydatiform mole, placental site tumor)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas (NHLs), indolent non-Hodgkin's lymphoma (iNHL), refractory iNHL, pituitary tumors;
eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma; large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumors (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas);
skin (e.g., clear cell hidradenoma, cutaneous benign fibrous histiocytomas, cylindroma, hidradenoma, melanoma (including cutaneous melanoma, mucosal melanoma), pilomatricoma, Spitz tumors); and
soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

In some embodiments, the KRAS G12C, G12D and/or G12V associated disease or condition is a cancer selected from a lung cancer, a colorectal cancer, a breast cancer, a prostate cancer, a cervical cancer, a pancreatic cancer and a head and neck cancer. In some embodiments, the cancer is metastatic.

In some embodiments, the KRAS G12C, G12D and/or G12V associated disease or condition is a cancer selected from non-small lung cancer (NSCLC), melanoma, triple-negative breast cancer (TNBC), nasopharyngeal cancer (NPC), microsatellite stable colorectal cancer (mssCRC), thymoma, and gastrointestinal stromal tumor (GIST). In some embodiments, the cancer is metastatic.

In some embodiments, the KRAS G12C, G12D and/or G12V associated disease or condition is a cancer of pancreatic cancer, bladder cancer, colorectal cancer, breast cancer, prostate cancer, renal cancer, hepatocellular cancer, lung cancer, ovarian cancer, cervical cancer, uterine cancer, gastric cancer, bile duct cancer, testicular cancer, esophageal cancer, head and neck cancer, melanoma, neuroendocrine cancer, CNS cancer, brain cancer, bone cancer, soft tissue sarcoma, non-small cell lung cancer, small-cell lung cancer, myelodysplastic syndrome, thyroid cancer, or colon cancer.

In some embodiments, the KRAS G12C, G12D and/or G12V associated disease or condition is a cancer of pancreatic cancer, colorectal cancer, non-small cell lung cancer, endometrial cancer, uterine endometrical carcinoma, cholangio carcinoma, testicular germ cell cancer, cervical squamous carcinoma, or myelodysplastic syndrome.

In some embodiments, the cancer is or myelodysplastic syndrome. In some embodiments, the cancer is high risk myelodysplastic syndrome or low risk myelodysplastic syndrome. In some embodiments, the cancer is high risk myelodysplastic syndrome. In some embodiments, the cancer is high risk myelodysplastic syndrome.

In some embodiments, the cancer is colorectal cancer. In some embodiments, the cancer is non-small cell lung cancer. In some embodiments, the cancer is pancreatic cancer. In some embodiments, the cancer is endometrial cancer. In some embodiments, the cancer is uterine endometrical carcinoma. In some embodiments, the cancer is testicular germ cell cancer. In some embodiments, the cancer is cervical squamous carcinoma. In some embodiments, the cancer is cholangio carcinoma.

The effective dosage of active ingredient employed may vary depending on the particular compound employed, the mode of administration, the condition being treated and the severity of the condition being treated. Such dosage may be ascertained readily by a person skilled in the art.

When treating or preventing a KRAS G12C, G12D and/or G12V associated disease or condition for which compounds of the present disclosure are indicated, generally satisfactory results are obtained when the compounds of the present disclosure are administered at a daily dosage of from about 0.1 milligram to about 300 milligram per kilogram of animal body weight.

In some embodiments, the compounds of the present disclosure are given as a single daily dose or in divided doses two to six times a day, or in sustained release form. For most large mammals, the total daily dosage is from about 1 milligram to about 1000 milligrams, or from about 1 milligram to about 50 milligrams. In the case of a 70 kg adult human, the total daily dose will generally be from about 0.1 milligrams to about 200 milligrams. This dosage regimen may be adjusted to provide the optimal therapeutic response. In some embodiments, the total daily dosage is from about 1 milligram to about 900 milligrams, about 1 milligram to about 800 milligrams, about 1 milligram to about 700 milligrams, about 1 milligram to about 600 milligrams, about 1 milligram to about 400 milligrams, about 1 milligram to about 300 milligrams, about 1 milligram to about 200 milligrams, about 1 milligram to about 100 milligrams, about 1 milligram to about 50 milligrams, about 1 milligram to about 20 milligram, or about 1 milligram to about 10 milligrams.

The compounds of the present application or the compositions thereof may be administered once, twice, three, or four times daily, using any suitable mode described above.

Also, administration or treatment with the compounds may be continued for a number of days; for example, commonly treatment would continue for at least 7 days, 14 days, or 28 days, for one cycle of treatment. Treatment cycles are frequently alternated with resting periods of about 1 to 28 days, commonly about 7 days or about 14 days, between cycles. The treatment cycles, in other embodiments, may also be continuous.

In some embodiments, the methods provided herein comprise administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, the compound or pharmaceutically acceptable salt thereof of the present disclosure is administered in combination with one or more additional therapeutic agent or therapeutic modality.

In some embodiments, the present disclosure provides the pharmaceutical composition or the method wherein the one or more additional therapeutic agent or additional therapeutic modality comprises one, two, three, or four additional therapeutic agents and/or therapeutic modalities.

In some embodiments, the present disclosure provides the pharmaceutical composition or the method wherein the additional therapeutic agent or therapeutic modalities are selected from an immune checkpoint modulator, an antibody-drug conjugate (ADC), an antiapoptotic agent, a targeted anticancer therapeutic, a chemotherapeutic agent, surgery, or radiation therapy.

In some embodiments, the present disclosure provides the pharmaceutical composition or the method wherein the immune checkpoint modulator is selected from an anti-PD-(L)1 antibody, an anti-TIGIT antibody, an anti-CTLA4 antibody, an anti-CCR8 antibody, an anti-TREM1 antibody, an anti-TREM2 antibody, a CD47 inhibitor, a DGKax inhibitor, an HPK1 inhibitor, a FLT3 agonist, an adenosine receptor antagonist, a CD39 inhibitor, a CD73 inhibitor, an IL-2 variant (IL-2v), and a CAR-T cell therapy.

In some embodiments, the present disclosure provides the pharmaceutical composition or the method wherein the anti-PD-(L)1 antibody is selected from pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab.

In some embodiments, the present disclosure provides the pharmaceutical composition or the method wherein the anti-TIGIT antibody is selected from tiragolumab, vibostolimab, domvanalimab, AB308, AK127, BMS-986207, and etigilimab.

In some embodiments, the present disclosure provides the pharmaceutical composition or the method wherein the anti-CTLA4 antibody is selected from ipilimumab, tremelimumab, and zalifrelimab.

In some embodiments, the present disclosure provides the pharmaceutical composition or the method wherein the CD47 inhibitor is selected from magrolimab, letaplimab, lemzoparlimab, AL-008, RRx-001, CTX-5861, FSI-189 (GS-0189), ES-004, BI-765063, ADU1805, CC-95251, and Q-1801.

In some embodiments, the present disclosure provides the pharmaceutical composition or the method wherein the adenosine receptor antagonist is etrumadenant (AB928), taminadenant, TT-10, TT-4, or M1069.

In some embodiments, the present disclosure provides the pharmaceutical composition or the method wherein the CD39 inhibitor is TTX-030.

In some embodiments, the present disclosure provides the pharmaceutical composition or the method wherein the CD73 inhibitor is quemliclustat (AB680), uliledlimab, mupadolimab, ORIC-533, ATG-037, PT-199, AK131, NZV930, BMS-986179, or oleclumab.

In some embodiments, the present disclosure provides the pharmaceutical composition or the method wherein the IL-2v is aldesleukin (Proleukin), bempegaldesleukin (NKTR-214), nemvaleukin alfa (ALKS-4230), THOR-202 (SAR-444245), BNT-151, ANV-419, XTX-202, RG-6279 (RO-7284755), NL-201, STK-012, SHR-1916, or GS-4528.

In some embodiments, the present disclosure provides the pharmaceutical composition or the method wherein the ADC is selected from sacituzumab govitecan, datopotamab deruxtecan, enfortumab vedotin, and trastuzumab deruxtecan.

In some embodiments, the present disclosure provides the pharmaceutical composition or the method wherein the additional therapeutic agent is selected from idealisib, sacituzumab govitecan, magrolimab, GS-0189, GS-3583, zimberelimab, GS-4224, GS-9716, GS-6451, GS-1811 (JTX-1811), quemliclustat (AB680), etrumadenant (AB928), domvanalimab, AB308, PY159, PY314, AGEN-1223, AGEN-2373, axicabtagene ciloleucel and brexucabtagene autoleucel.

In some embodiments, the method includes administering one or more additional therapeutic agents. The one or more additional therapeutic agents can be one or more therapeutic agents as described below. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

In some embodiments, the one or more additional therapeutic agents includes therapeutic agents used to treat high risk myelodysplastic syndrome (HR MDS), low risk myelodyplastic syndrome (LR MDS), colorectal cancer, non-small cell lung cancer (NSCLC), pancreatic cancer, or endometrial cancer. In some embodiments, the one or more additional therapeutic agents includes therapeutic agents used to treat high risk myelodysplastic syndrome (HR MDS). In some embodiments, the one or more additional therapeutic agents includes azacitidine (Vidaza®), decitabine (Dacogen®), lenalidomide (Revlimid®), cytarabine, idarubicin, daunorubicin, cytarabine+daunorubicin, cytarabine+idarubicin, pevonedistat, venetoclax, sabatolimab, guadecitabine, rigosertib, ivosidenib, enasidenib, selinexor, BGB324, DSP-7888, or SNS-301.

In some embodiments, the one or more additional therapeutic agents includes therapeutic agents used to treat low risk myelodyplastic syndrome (LR MDS). In some embodiments, the one or more additional therapeutic agents includes lenalidomide, azacytidine, roxadustat, luspatercept, imetelstat, LB-100, or rigosertib.

In some embodiments, the one or more additional therapeutic agents includes therapeutic agents used to treat colorectal cancer. In some embodiments, the one or more additional therapeutic agents includes bevacizumab, capecitabine, cetuximab, fluorouracil, irinotecan, leucovorin, oxaliplatin, panitumumab, ziv-aflibercept, bevacizumab (Avastin®), leucovorin, 5-FU, oxaliplatin (FOLFOX), pembrolizumab (Keytruda®), FOLFIRI, regorafenib (Stivarga®), aflibercept (Zaltrap®), cetuximab (Erbitux®), Lonsurf (Orcantas®), XELOX, FOLFOXIRI, bevacizumab+leucovorin+5-FU+oxaliplatin (FOLFOX), bevacizumab+FOLFIRI, bevacizumab+FOLFOX, aflibercept+FOLFIRI, cetuximab+FOLFIRI, bevacizumab+XELOX, bevacizumab+FOLFOXIRI, binimetinib+encorafenib+cetuximab, trametinib+dabrafenib+panitumumab, trastuzumab+pertuzumab, napabucasin+FOLFIRI+bevacizumab, or nivolumab+ipilimumab.

In some embodiments, the one or more additional therapeutic agents includes therapeutic agents used to treat non-small cell lung cancer (NSCLC). In some embodiments, the one or more additional therapeutic agents includes afatinib, albumin-bound paclitaxel, alectinib, atezolizumab, bevacizumab, bevacizumab, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, trametinib, trastuzumab, vandetanib, vemurafenib, vinblastine, vinorelbine, alectinib (Alecensa®), dabrafenib (Tafinlar®), trametinib (Mekinist®), osimertinib (Tagrisso®), entrectinib (Tarceva®), crizotinib (Xalkori®), pembrolizumab (Keytruda®), carboplatin, pemetrexed (Alimta®), nab-paclitaxel (Abraxane®), ramucirumab (Cyramza®), docetaxel, bevacizumab (Avastin®), brigatinib, gemcitabine, cisplatin, afatinib (Gilotrif®), nivolumab (Opdivo®), gefitinib (Iressa®), dabrafenib+trametinib, pembrolizumab+carboplatin+pemetrexed, pembrolizumab+carboplatin+nab-paclitaxel, ramucirumab+docetaxel, bevacizumab+carboplatin+pemetrexed, pembrolizumab+pemetrexed+carboplatin, cisplatin+pemetrexed, bevacizumab+carboplatin+nab-paclitaxel, cisplatin+gemcitabine, nivolumab+docetaxel, carboplatin+pemetrexed, carboplatin+nab-paclitaxel, or pemetrexed+cisplatin+carboplatin, datopotamab deruxtecan (DS-1062), trastuzumab deruxtecan (Enhertu®), enfortumab vedotin (Padcev®), durvalumab, canakinumab, cemiplimab, nogapendekin alfa, avelumab, tiragolumab, domvanalimab, vibostolimab, ociperlimab, datopotamab deruxtecan+pembrolizumab, datopotamab deruxtecan+durvalumab, durvalumab+tremelimumab, pembrolizumab+lenvatinib+pemetrexed, pembrolizumab+olaparib, nogapendekin alfa (N-803)+pembrolizumab, tiragolumab+atezolizumab, vibostolimab+pembrolizumab, or ociperlimab+tislelizumab.

In some embodiments, the one or more additional therapeutic agents includes therapeutic agents used to treat pancreatic cancer. In some embodiments, the one or more additional therapeutic agents includes 5-FU, leucovorin, oxaliplatin, irinotecan, gemcitabine, nab-paclitaxel (Abraxane®), FOLFIRINOX, 5-FU+leucovorin+oxaliplatin+irinotecan, 5-FU+nanoliposomal irinotecan, leucovorin+nanoliposomal irinotecan, or gemcitabine+nab-paclitaxel.

In some embodiments, the one or more additional therapeutic agents includes therapeutic agents used to treat endometrial cancer. In some embodiments, the one or more additional therapeutic agents includes carboplatin, paclitaxel, cisplatin, doxorubicin, ifosfamide, progesterone, anastrozole (Arimidex®), letrozole (Femara®), exemestane (Aromasin®), pembrolizumab (Keytruda®), lenvatinib (Lenvima®), or dostarlimab (Jemperli®).

In some embodiments, the one or more additional therapeutic agents is independently SNS-301, 5-FU+leucovorin+oxaliplatin+irinotecan, 5-FU+nanoliposomal irinotecan, 5-FU, afatinib (Gilotrif®), aflibercept (Zaltrap®), aflibercept+FOLFIRI, albumin-bound paclitaxel, alectinib (Alecensa®), anastrozole (Arimidex®), atezolizumab, avelumab, azacitidine (Vidaza®), bevacizumab (Avastin®), bevacizumab+carboplatin+nab-paclitaxel, bevacizumab+carboplatin+pemetrexed, bevacizumab+FOLFIRI, bevacizumab+FOLFOX, bevacizumab+FOLFOXIRI, bevacizumab+leucovorin+5-FU+oxaliplatin (FOLFOX), bevacizumab+XELOX, bevacizumab, BGB324, binimetinib+encorafenib+cetuximab, brigatinib, cabozantinib, canakinumab, capecitabine, carboplatin+nab-paclitaxel, carboplatin+pemetrexed, carboplatin, cemiplimab, cetuximab (Erbitux®), cetuximab+FOLFIRI, cisplatin+gemcitabine, cisplatin+pemetrexed, cisplatin, crizotinib (Xalkori®), cytarabine+daunorubicin, cytarabine+idarubicin, cytarabine, dabrafenib (Tafinlar®), dabrafenib+trametinib, datopotamab deruxtecan (DS-1062), datopotamab deruxtecan+durvalumab, datopotamab deruxtecan+pembrolizumab, daunorubicin, decitabine (Dacogen®), docetaxel, domvanalimab, dostarlimab (Jemperli®), doxorubicin, DSP-7888, durvalumab+tremelimumab, durvalumab, enasidenib, enfortumab vedotin (Padcev®), entrectinib (Tarceva®), erlotinib, etoposide, exemestane (Aromasin®), fluorouracil, FOLFIRI, FOLFIRINOX, FOLFOXIRI, gefitinib (Iressa®), gemcitabine+nab-paclitaxel, gemcitabine, guadecitabine, idarubicin, ifosfamide, imetelstat, irinotecan, ivosidenib, LB-100, lenalidomide (Revlimid®), lenalidomide, lenvatinib (Lenvima®), letrozole (Femara®), leucovorin+nanoliposomal irinotecan, leucovorin, Lonsurf (Orcantas®), luspatercept, nab-paclitaxel (Abraxane®), napabucasin+FOLFIRI+bevacizumab, nivolumab (Opdivo®), nivolumab+docetaxel, nivolumab+ipilimumab, nogapendekin alfa (N-803)+pembrolizumab, nogapendekin alfa, ociperlimab+tislelizumab, ociperlimab, osimertinib (Tagrisso®), oxaliplatin (FOLFOX), paclitaxel, panitumumab, pembrolizumab (Keytruda®), pembrolizumab+carboplatin+nab-paclitaxel, pembrolizumab+carboplatin+pemetrexed, pembrolizumab+lenvatinib+pemetrexed, pembrolizumab+olaparib, pembrolizumab+pemetrexed+carboplatin, pemetrexed (Alimta®), pemetrexed+cisplatin+carboplatin, pevonedistat, progesterone, ramucirumab (Cyramza®), ramucirumab+docetaxel, regorafenib (Stivarga®), rigosertib, roxadustat, sabatolimab, selinexor, tiragolumab+atezolizumab, tiragolumab, trametinib (Mekinist®), trametinib+dabrafenib+panitumumab, trastuzumab+pertuzumab, trastuzumab deruxtecan (Enhertu®), trastuzumab, vandetanib, vemurafenib, venetoclax, vibostolimab+pembrolizumab, vibostolimab, vinblastine, vinorelbine, XELOX, or ziv-aflibercept.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

### VIII. Combination Therapy

In some embodiments, a compound of Formula (I), (Ia), and/or (Ib), provided herein, or pharmaceutically acceptable salt thereof, is administered in combination with one or more additional therapeutic agents to treat or prevent a disease or condition disclosed herein. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, the pharmaceutical compositions provided herein have a compound of Formula (I), (Ia), and/or (Ib), provided herein, or pharmaceutically acceptable salt thereof, and one or more additional therapeutic agents. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents.

In some embodiments, the one or more additional therapeutic agents are three additional therapeutic agents. In some embodiments, the one or more additional therapeutic agents are four additional therapeutic agents.

In some embodiments the additional therapeutic agent includes, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (e.g., a mono- and multi-specific antibody, or fragment thereof, in any format, such as DART®, Duobody®, BiTE®, BiKE, TriKE, XmAb®, TandAb®, scFv, Fab, Fab derivative), a bi-specific antibody, a non-immunoglobulin antibody mimetic (e.g., including adnectin, affibody, affilin, affimer, affitin, alphabody, anticalin, peptide aptamer, armadillo repeat protein (ARM), atrimer, avimer, designed ankyrin repeat protein (DARPin®), fynomer, knottin, Kunitz domain peptide, monobody, and nanoCLAMPs), an antibody-drug conjugate (ADC), antibody-peptide conjugate), an oncolytic virus, a gene modifier or editor, a cell comprising a chimeric antigen receptor (CAR), e.g., including a T-cell immunotherapeutic agent, an NK-cell immunotherapeutic agent, or a macrophage immunotherapeutic agent, a cell comprising an engineered T-cell receptor (TCR-T), or any combination thereof.

### Illustrative Targets

In some embodiments, the one or more additional therapeutic agents include, e.g., an inhibitor, agonist, antagonist, ligand, modulator, stimulator, blocker, activator or suppressor of a target (e.g., polypeptide or polynucleotide), such as: 2′-5′-oligoadenylate synthetase (OAS1; NCBI Gene ID: 4938); 5′-3′ exoribonuclease 1 (XRN1; NCBI Gene ID: 54464); 5′-nucleotidase ecto (NT5E, CD73; NCBI Gene ID: 4907); ABL proto-oncogene 1, non-receptor tyrosine kinase (ABL1, BCR-ABL, c-ABL, v-ABL; NCBI Gene ID: 25); absent in melanoma 2 (AIM2; NCBI Gene ID: 9447); acetyl-CoA acyltransferase 2 (ACAA2; NCBI Gene ID: 10499); acid phosphatase 3 (ACP3; NCBI Gene ID: 55); adenosine deaminase (ADA, ADA1; NCBI Gene ID: 100); adenosine receptors (e.g., ADORA1 (A1), ADORA2A (A2a, A2AR), ADORA2B (A2b, A2BR), ADORA3 (A3); NCBI Gene IDs: 134, 135, 136, 137); AKT serine/threonine kinase 1 (AKT1, AKT, PKB; NCBI Gene ID: 207); alanyl aminopeptidase, membrane (ANPEP, CD13; NCBI Gene ID: 290); ALK receptor tyrosine kinase (ALK, CD242; NCBI Gene ID: 238); alpha fetoprotein (AFP; NCBI Gene ID: 174); amine oxidase copper containing (e.g., AOC1 (DA01), AOC2, AOC3 (VAP1); NCBI Gene IDs: 26, 314, 8639); androgen receptor (AR; NCBI Gene ID: 367); angiopoietins (ANGPT1, ANGPT2; NCBI Gene IDs: 284, 285); angiotensin II receptor type 1 (AGTR1; NCBI Gene ID: 185); angiotensinogen (AGT; NCBI Gene ID: 183); apolipoprotein A1 (APOA1; NCBI Gene ID: 335); apoptosis inducing factor mitochondria associated 1 (AIFM1, AIF; NCBI Gene ID: 9131); arachidonate 5-lipoxygenase (ALOX5; NCBI Gene ID: 240); asparaginase (ASPG; NCBI Gene ID: 374569); asteroid homolog 1 (ASTE1; NCBI Gene ID: 28990); ATM serine/threonine kinase (ATM; NCBI Gene ID: 472); ATP binding cassette subfamily B member 1 (ABCB1, CD243, GP170; NCBI Gene ID: 5243); ATP-dependent Clp-protease (CLPP; NCBI Gene ID: 8192); ATR serine/threonine kinase (ATR; NCBI Gene ID: 545); AXL receptor tyrosine kinase (AXL; NCBI Gene ID: 558); B and T lymphocyte associated (BTLA, CD272; NCBI Gene ID: 151888); baculoviral IAP repeat containing proteins (BIRC2 (cIAP1), BIRC3 (cIAP2), XIAP (BIRC4, IAP3), BIRC5 (survivin); NCBI Gene IDs: 329, 330, 331, 332); basigin (Ok blood group) (BSG, CD147; NCBI Gene ID: 682); B-cell lymphoma 2 (BCL2; NCBI Gene ID: 596); BCL2 binding component 3 (BBC3, PUMA; NCBI Gene ID: 27113); BCL2 like (e.g., BCL2L1 (Bcl-x), BCL2L2 (BIM); Bcl-x; NCBI Gene IDs: 598, 10018); beta 3-adrenergic receptor (ADRB3; NCBI Gene ID: 155); bone gamma-carboxyglutamate protein (BGLAP; NCBI Gene ID: 632); bone morphogenetic protein-10 ligand (BMP10; NCBI Gene ID: 27302); bradykinin receptors (e.g., BDKRB1, BDKRB2; NCBI Gene IDs: 623, 624); B-RAF (BRAF; NCBI Gene ID: 273); breakpoint cluster region (BCR; NCBI Gene ID: 613); bromodomain and external domain (BET) bromodomain containing proteins (e.g., BRD2, BRD3, BRD4, BRDT; NCBI Gene IDs: 6046, 8019, 23476, 676); Bruton's tyrosine kinase (BTK; NCBI Gene ID: 695); cadherins (e.g., CDH3 (p-cadherin), CDH6 (k-cadherin); NCBI Gene IDs: 1001, 1004); cancer/testis antigens (e.g., CTAG1A, CTAG1B, CTAG2; NCBI Gene IDs: 1485, 30848, 246100); cannabinoid receptors (e.g., CNR1 (CB1), CNR2 (CB2); NCBI Gene IDs: 1268, 1269); carbohydrate sulfotransferase 15 (CHST15; NCBI Gene ID: 51363); carbonic anhydrases (e.g., CA1, CA2, CA3, CA4, CA5A, CA5B, CA6, CA7, CA8, CA9, CA10, CA1l, CA12, CA13, CA14; NCBI Gene IDs: 759, 760, 761, 762, 763, 765, 766, 767, 768, 770, 771, 11238, 23632, 56934, 377677); carcinoembryonic antigen related cell adhesion molecules (e.g., CEACAM3 (CD66d), CEACAM5 (CD66e), CEACAM6 (CD66c); NCBI Gene IDs: 1048, 1084, 4680); casein kinases (e.g., CSNK1A1 (CK1), CSNK2A1 (CK2); NCBI Gene IDs: 1452, 1457); caspases (e.g., CASP3, CASP7, CASP8; NCBI Gene IDs: 836, 840, 841, 864); catenin beta 1 (CTNNB1; NCBI Gene ID: 1499); cathepsin G (CTSG; NCBI Gene ID: 1511); Cb1 proto-oncogene B (CBLB, Cb1-b; NCBI Gene ID: 868); C—C motif chemokine ligand 21 (CCL21; NCBI Gene ID: 6366); C—C motif chemokine receptor 2 (CCR2; NCBI Gene ID: 729230); C—C motif chemokine receptors (e.g., CCR3 (CD193), CCR4 (CD194), CCR5 (CD195), CCR8 (CDw198); NCBI Gene IDs: 1232, 1233, 1234, 1237); CCAAT enhancer binding protein alpha (CEBPA, CEBP; NCBI Gene ID: 1050); cell adhesion molecule 1 (CADM1; NCBI Gene ID: 23705); cell division cycle 7 (CDC7; NCBI Gene ID: 8317); cellular communication network factor 2 (CCN2; NCBI Gene ID: 1490); cereblon (CRBN; NCBI Gene ID: 51185); checkpoint kinases (e.g., CHEK1 (CHK1), CHEK2 (CHK2); NCBI Gene IDs: 1111, 11200); cholecystokinin B receptor (CCKBR; NCBI Gene ID: 887); chorionic somatomammotropin hormone 1 (CSH1; NCBI Gene ID: 1442); claudins (e.g., CLDN6, CLDN18; NCBI Gene IDs: 9074, 51208); cluster of differentiation markers (e.g., CD1A, CD1C, CD1D, CD1E, CD2, CD3 alpha (TRA), CD beta (TRB), CD gamma (TRG), CD delta (TRD), CD4, CD8A, CD8B, CD19, CD20 (MS4A1), CD22, CD24, CD25 (IL2RA, TCGFR), CD28, CD33 (SIGLEC3), CD37, CD38, CD39 (ENTPD1), CD40 (TNFRSF5), CD44 (MIC4, PGP1), CD47 (IAP), CD48 (BLAST1), CD52, CD55 (DAF), CD58 (LFA3), CD74, CD79a, CD79b, CD80 (B7-1), CD84, CD86 (B7-2), CD96 (TACTILE), CD99 (MIC2), CD115 (CSF1R), CD116 (GMCSFR, CSF2RA), CD122 (IL2RB), CD123 (IL3RA), CD128 (IL8R1), CD132 (IL2RG), CD135 (FLT3), CD137 (TNFRSF9, 4-1BB), CD142 (TF, TFA), CD152 (CTLA4), CD160, CD182 (IL8R2), CD193 (CCR3), CD194 (CCR4), CD195 (CCR5), CD207, CD221 (IGF1R), CD222 (IGF2R), CD223 (LAG3), CD226 (DNAM1), CD244, CD247, CD248, CD276 (B7-H3), CD331 (FGFR1), CD332 (FGFR2), CD333 (FGFR3), CD334 (FGFR4); NCBI Gene IDs: 909, 911, 912, 913, 914, 919, 920, 923, 925, 926, 930, 931, 933, 940, 941, 942, 945, 951, 952, 953, 958,960, 961, 962, 965, 972, 973, 974, 1043, 1232, 1233, 1234, 1237, 1436, 1438, 1493, 1604, 2152, 2260, 2261, 2263, 2322, 3480, 3482, 3559, 3560, 3561, 3563, 3577, 3579, 3604, 3902, 4267, 6955, 6957, 6964, 6965, 8832, 10666, 11126, 50489, 51744, 80381, 100133941); clusterin (CLU; NCBI Gene ID: 1191); coagulation factors (e.g., F7, FXA, NCBI Gene IDs: 2155, 2159); collagen type IV alpha chains (e.g., COL4A1, COL4A2, COL4A3, COL4A4, COL4A5; NCBI Gene IDs: 1282, 1284, 1285, 1286, 1287); collectin subfamily member 10 (COLEC10; NCBI Gene ID: 10584); colony stimulating factors (e.g., CSF1 (MCSF), CSF2 (GMCSF), CSF3 (GCSF); NCBI Gene IDs: 1435, 1437, 1440); complement factors (e.g., C3, C5; NCBI Gene IDs: 718, 727); COP9 signalosome subunit 5 (COPS5; NCBI Gene ID: 10987); C-type lectin domain family member (e.g., CLEC4C (CD303), CLEC9A (CD370), CLEC12A (CD371); CD371; NCBI Gene ID: 160364, 170482, 283420); C—X—C motif chemokine ligand 12 (CXCL12; NCBI Gene ID: 6387); C—X—C motif chemokine receptors (CXCR1 (IL8R1, CD128), CXCR2 (IL8R2, CD182), CXCR3 (CD182, CD183, IP-10R), CXCR4 (CD184); NCBI Gene ID: 2833, 3577, 3579, 7852); cyclin D1 (CCND1, BCL1; NCBI Gene ID: 595); cyclin dependent kinases (e.g., CDK1, CDK2, CDK3, CDK4, CDK5, CDK6, CDK7, CDK8, CDK9, CDK10, CDK12; NCBI Gene ID: 983, 1017, 1018, 1019, 1020, 1021, 1022, 1024, 1025, 8558, 51755); cyclin G1 (CCNG1; NCBI Gene ID: 900); cytochrome P450 family members (e.g., CYP2D6, CYP3A4, CYP11A1, CYP11B2, CYP17A1, CYP19A1, CYP51A1; NCBI Gene IDs: 1565, 1576, 1583, 1585, 1586, 1588, 1595); cytochrome P450 oxidoreductase (POR; NCBI Gene ID: 5447); cytokine inducible SH2 containing protein (CISH; NCBI Gene ID: 1154); cytotoxic T-lymphocyte associated protein 4 (CTLA4, CD152; NCBI Gene ID: 1493); DEAD-box helicases (e.g., DDX5, DDX6, DDX58; NCBI Gene IDs: 1655, 1656, 23586); delta like canonical Notch ligands (e.g., DLL3, DLL4; NCBI Gene IDs: 10683, 54567); diablo IAP-binding mitochondrial protein (DIABLO, SMAC; NCBI Gene ID: 56616); diacylglycerol kinases (e.g., DGKA, DGKZ; NCBI Gene IDs: 1606, 8525); dickkopf WNT signaling pathway inhibitors (e.g., DKK1, DKK3; NCBI Gene ID: 22943, 27122); dihydrofolate reductase (DHFR; NCBI Gene ID: 1719); dihydropyrimidine dehydrogenase (DPYD; NCBI Gene ID: 1806); dipeptidyl peptidase 4 (DPP4; NCBI Gene ID: 1803); discoidin domain receptor tyrosine kinases (e.g., DDR1 (CD167), DDR2; CD167; NCBI Gene ID: 780, 4921); DNA dependent protein kinase (PRKDC; NCBI Gene ID: 5591); DNA topoisomerases (e.g., TOP1, TOP2A, TOP2B, TOP3A, TOP3B; NCBI Gene ID: 7150, 7153, 7155, 7156, 8940); dopachrome tautomerase (DCT; NCBI Gene ID: 1638); dopamine receptor D2 (DRD2; NCBI Gene ID: 1318); DOT1 like histone lysine methyltransferase (DOT1L; NCBI Gene ID: 84444); ectonucleotide pyrophosphatase/phosphodiesterase 3 (ENPP3, CD203c; NCBI Gene ID: 5169); EMAP like 4 (EML4; NCBI Gene ID: 27436); endoglin (ENG; NCBI Gene ID: 2022); endoplasmic reticulum aminopeptidases (e.g., ERAP1, ERAP2; NCBI Gene ID: 51752, 64167); enhancer of zeste 2 polycomb repressive complex 2 subunit (EZH2; NCBI Gene ID: 2146); ephrin receptors (e.g., EPHA1, EPHA2EPHA3, EPHA4, EPHA5, EPHA7, EPHB4; NCBIGene ID:1969, 2041, 2042, 2043, 2044, 2045, 2050); ephrins (e.g., EFNA1, EFNA4, EFNB2; NCBI Gene ID: 1942, 1945, 1948); epidermal growth factor receptors (e.g., ERBB1 (HER1, EGFR), ERBB1 variant III (EGFRvIII), ERBB2 (HER2, NEU, CD340), ERBB3 (HER3), ERBB4 (HER4); NCBI Gene ID: 1956, 2064, 2065, 2066); epithelial cell adhesion molecule (EPCAM; NCBI Gene ID: 4072); epithelial mitogen (EPGN; NCBI Gene ID: 255324); eukaryotic translation elongation factors (e.g., EEF1A2, EEF2; NCBI Gene ID: 1917, 1938); eukaryotic translation initiation factors (e.g., EIF4A1, EIF5A; NCBI Gene ID: 1973, 1984); exportin-1 (XPO1; NCBI Gene ID: 7514); farnesoid X receptor (NR1H4, FXR; NCBI Gene ID: 9971); Fas ligand (FASLG, FASL, CD95L, CD178, TNFSF6; NCBI Gene ID: 356); fatty acid amide hydrolase (FAAH; NCBI Gene ID: 2166); fatty acid synthase (FASN; FAS; NCBI Gene ID: 2194); Fe fragment of Ig receptors (e.g., FCER1A, FCGRT, FCGR3A (CD16); NCBI Gene IDs: 2205, 2214, 2217); Fe receptor like 5 (FCRL5, CD307; NCBI Gene ID: 83416); fibroblast activation protein alpha (FAP; NCBI Gene ID: 2191); fibroblast growth factor receptors (e.g., FGFR1 (CD331), FGFR2 (CD332), FGFR3 (CD333), FGFR4 (CD334); NCBI Gene IDs: 2260, 2261, 2263, 2264); fibroblast growth factors (e.g., FGF1 (FGF alpha), FGF2 (FGF beta), FGF4, FGF5; NCBI Gene IDs: 2246, 2247, 2249, 2250); fibronectin 1 (FN1, MSF; NCBI Gene ID: 2335); fms related receptor tyrosine kinases (e.g., FLT1 (VEGFR1), FLT3 (STK1, CD135), FLT4 (VEGFR2); NCBI Gene IDs: 2321, 2322, 2324); fms related receptor tyrosine kinase 3 ligand (FLT3LG; NCBI Gene ID: 2323); focal adhesion kinase 2 (PTK2, FAK1; NCBI Gene ID: 5747); folate hydrolase 1 (FOLH1, PSMA; NCBI Gene ID: 2346); folate receptor 1 (FOLR1; NCBI Gene ID: 2348); forkhead box protein M1 (FOXM1; NCBI Gene ID: 2305); FURIN (FURIN, PACE; NCBI Gene ID: 5045); FYN tyrosine kinase (FYN, SYN; NCBI Gene ID: 2534); galectins (e.g., LGALS3, LGALS8 (PCTA1), LGALS9; NCBI Gene ID: 3958, 3964, 3965); glucocorticoid receptor (NR3C1, GR; NCBI Gene ID: 2908); glucuronidase beta (GUSB; NCBI Gene ID: 2990); glutamate metabotropic receptor 1 (GRM1; NCBI Gene ID: 2911); glutaminase (GLS; NCBI Gene ID: 2744); glutathione S-transferase Pi (GSTP1; NCBI Gene ID: 2950); glycogen synthase kinase 3 beta (GSK3B; NCBI Gene ID: 2932); glypican 3 (GPC3; NCBI Gene ID: 2719); gonadotropin releasing hormone 1 (GNRH1; NCBI Gene ID: 2796); gonadotropin releasing hormone receptor (GNRHR; NCBI Gene ID: 2798); GPNMB glycoprotein nmb (GPNMB, osteoactivin; NCBI Gene ID: 10457); growth differentiation factor 2 (GDF2, BMP9; NCBI Gene ID: 2658); growth factor receptor-bound protein 2 (GRB2, ASH; NCBI Gene ID: 2885); guanylate cyclase 2C (GUCY2C, STAR, MECIL, MUCIL, NCBI Gene ID: 2984); H19 imprinted maternally expressed transcript (H19; NCBI Gene ID: 283120); HCK proto-oncogene, Src family tyrosine kinase (HCK; NCBI Gene ID: 3055); heat shock proteins (e.g., HSPA5 (HSP70, BIP, GRP78), HSPB1 (HSP27), HSP90B1 (GP96); NCBI Gene IDs: 3309, 3315, 7184); heme oxygenases (e.g., HMOX1 (HO1), HMOX2 (HO1); NCBI Gene ID: 3162, 3163); heparanase (HPSE; NCBI Gene ID: 10855); hepatitis A virus cellular receptor 2 (HAVCR2, TIM3, CD366; NCBI Gene ID: 84868); hepatocyte growth factor (HGF; NCBI Gene ID: 3082); HERV-H LTR-associating 2 (HHLA2, B7-H7; NCBI Gene ID: 11148); histamine receptor H2 (HRH2; NCBI Gene ID: 3274); histone deacetylases (e.g., HDAC1, HDAC7, HDAC9; NCBI Gene ID: 3065, 9734, 51564); HRas proto-oncogene, GTPase (HRAS; NCBI Gene ID: 3265); hypoxia-inducible factors (e.g., HIF1A, HIF2A (EPAS1); NCBI Gene IDs: 2034, 3091); I-Kappa-B kinase (IKK beta; NCBI Gene IDs: 3551, 3553); IKAROS family zinc fingers (IKZF1 (LYF1), IKZF3; NCBI Gene ID: 10320, 22806); immunoglobulin superfamily member 11 (IGSF11; NCBI Gene ID: 152404); indoleamine 2,3-dioxygenases (e.g., IDO1, IDO2; NCBI Gene IDs: 3620, 169355); inducible T cell costimulator (ICOS, CD278; NCBI Gene ID: 29851); inducible T cell costimulator ligand (ICOSLG, B7-H2; NCBI Gene ID: 23308); insulin like growth factor receptors (e.g., IGF1R, IGF2R; NCBI Gene ID: 3480, 3482); insulin like growth factors (e.g., IGF1, IGF2; NCBI Gene IDs: 3479, 3481); insulin receptor (INSR, CD220; NCBI Gene ID: 3643); integrin subunits (e.g., ITGA5 (CD49e), ITGAV (CD51), ITGB1 (CD29), ITGB2 (CD18, LFA1, MAC1), ITGB7; NCBI Gene IDs: 3678, 3685, 3688, 3695, 3698); intercellular adhesion molecule 1 (ICAM1, CD54; NCBI Gene ID: 3383); interleukin 1 receptor associated kinase 4 (IRAK4; NCBI Gene ID: 51135); interleukin receptors (e.g., IL2RA (TCGFR, CD25), IL2RB (CD122), IL2RG (CD132), IL3RA, IL6R, IL13RA2 (CD213A2), IL22RA1; NCBI Gene IDs: 3598, 3559, 3560, 3561, 3563, 3570, 58985); interleukins (e.g., IL1A, IL1B, IL2, IL3, IL6 (HGF), IL7, IL8 (CXCL8), IL10 (TGIF), IL12A, IL12B, IL15, IL17A (CTLA8), IL18, IL23A, IL24, IL-29 (IFNL1); NCBI Gene IDs: 3552, 3553, 3558, 3562, 3565, 3569, 3574, 3586, 3592, 3593, 3600, 3605, 3606, 11009, 51561, 282618); isocitrate dehydrogenases (NADP(+)1) (e.g., IDH1, IDH2; NCBI Gene IDs: 3417, 3418); Janus kinases (e.g., JAK1, JAK2, JAK3; NCBI Gene IDs: 3716, 3717, 3718); kallikrein related peptidase 3 (KLK3; NCBI Gene ID: 354); killer cell immunoglobulin like receptor, Ig domains and long cytoplasmic tails (e.g., KIR2DL1 (CD158A), KIR2DL2 (CD158B1), KIR2DL3 (CD158B), KIR2DL4 (CD158D), KIR2DL5A (CD158F), KIR2DL5B, KIR3DL1 (CD158E1), KIR3DL2 (CD158K), KIR3DP1 (CD158c), KIR2DS2 (CD158J); NCBI Gene IDs: 3802, 3803, 3804, 3805, 3811, 3812, 57292, 553128, 548594, 100132285); killer cell lectin like receptors (e.g., KLRC1 (CD159A), KLRC2 (CD159c), KLRC3, KLRRC4, KLRD1 (CD94), KLRG1, KLRK1 (NKG2D, CD314); NCBI Gene IDs: 3821, 3822, 3823, 3824, 8302, 10219, 22914); kinase insert domain receptor (KDR, CD309, VEGFR2; NCBI Gene ID: 3791); kinesin family member 11 (KIF11; NCBI Gene ID: 3832); KiSS-1 metastasis suppressor (KISS1; NCBI Gene ID: 3814); KIT proto-oncogene, receptor tyrosine kinase (KIT, C-KIT, CD117; NCBI Gene ID: 3815); KRAS proto-oncogene, GTPase (KRAS; NCBI Gene ID: 3845); lactotransferrin (LTF; NCBI Gene ID: 4057); LCK proto-oncogene, Src family tyrosine kinase (LCK; NCBI Gene ID: 3932); LDL receptor related protein 1 (LRP1, CD91, IGFBP3R; NCBI Gene ID: 4035); leucine rich repeat containing 15 (LRRC15; NCBI Gene ID: 131578); leukocyte immunoglobulin like receptors (e.g., LILRB1 (ILT2, CD85J), LILRB2 (ILT4, CD85D); NCBI Gene ID: 10288, 10859); leukotriene A4 hydrolase (LTA4H; NCBI Gene ID: 4048); linker for activation of T-cells (LAT; NCBI Gene ID: 27040); luteinizing hormone/choriogonadotropin receptor (LHCGR; NCBI Gene ID: 3973); LY6/PLAUR domain containing 3 (LYPD3; NCBI Gene ID: 27076); lymphocyte activating 3 (LAG3; CD223; NCBI Gene ID: 3902); lymphocyte antigens (e.g., LY9 (CD229), LY75 (CD205); NCBI Gene IDs: 4063, 17076); LYN proto-oncogene, Src family tyrosine kinase (LYN; NCBI Gene ID: 4067); lypmphocyte cytosolic protein 2 (LCP2; NCBI Gene ID: 3937); lysine demethylase 1A (KDM1A; NCBI Gene ID: 23028); lysophosphatidic acid receptor 1 (LPAR1, EDG2, LPA1, GPR26; NCBI Gene ID: 1902); lysyl oxidase (LOX; NCBI Gene ID: 4015); lysyl oxidase like 2 (LOXL2; NCBI Gene ID: 4017); macrophage migration inhibitory factor (MIF, GIF; NCBI Gene ID: 4282); macrophage stimulating 1 receptor (MST1R, CD136; NCBI Gene ID: 4486); MAGE family members (e.g., MAGEA1, MAGEA2, MAGEA2B, MAGEA3, MAGEA4, MAGEA5, MAGEA6, MAGEA10, MAGEA11, MAGEC1, MAGEC2, MAGED1, MAGED2; NCBI Gene IDs: 4100, 4101, 4102, 4103, 4104, 4105, 4109, 4110, 9500, 9947, 10916, 51438, 266740); major histocompatibility complexes (e.g., HLA-A, HLA-E, HLA-F, HLA-G; NCBI Gene IDs: 3105, 3133, 3134, 3135); major vault protein (MVP, VAULT1; NCBI Gene ID: 9961); MALT1 paracaspase (MALT1; NCBI Gene ID: 10892); MAPK activated protein kinase 2 (MAPKAPK2; NCBI Gene ID: 9261); MAPK interacting serine/threonine kinases (e.g., MKNK1, MKNK2; NCBI Gene IDs: 2872, 8569); matrix metallopeptidases (e.g., MMP1, MMP2, MMP3, MMP7, MMP8, MMP9, MMP10, MMP11, MMP12, MMP13, MMP14, MMP15, MMP16, MMP17, MMP19, MMP20, MMP21, MMP24, MMP25, MMP26, MMP27, MMP28; NCBI Gene IDs: 4312, 4313, 4314, 4316, 4317, 4318, 4319, 4320, 4321, 4322, 4323, 4324, 4325, 4326, 4327, 9313, 10893, 56547, 64066, 64386, 79148, 118856); MCL1 apoptosis regulator, BCL2 family member (MCL1; NCBI Gene ID: 4170); MDM2 proto-oncogene (MDM2; NCBI Gene ID: 4193); MDM4 regulator of p53 (MDM4; BMFS6; NCBI Gene ID: 4194); mechanistic target of rapamycin kinase (MTOR, FRAP1; NCBI Gene ID: 2475); melan-A (MLANA; NCBI Gene ID: 2315); melanocortin receptors (MC1R, MC2R; NCBI Gene IDs: 4157, 4148); MER proto-oncogene, tyrosine kinase (MERTK; NCBI Gene ID: 10461); mesothelin (MSLN; NCBI Gene ID: 10232); MET proto-oncogene, receptor tyrosine kinase (MET, c-Met, HGFR; NCBI Gene ID: 4233); methionyl aminopeptidase 2 (METAP2, MAP2; NCBI Gene ID: 10988); MHC class I polypeptide-related sequences (e.g., MICA, MICB; NCBI Gene IDs: 4277, 100507436); mitogen activated protein kinases (e.g., MAPK1 (ERK2), MAPK3 (ERK1), MAPK8 (JNK1), MAPK9 (JNK2), MAPK10 (JNK3), MAPK11 (p38 beta), MAPK12; NCBI Gene IDs: 5594, 5595, 5599, 5600, 5601, 5602, 819251); mitogen-activated protein kinase kinase kinases (e.g., MAP3K5 (ASK1), MAP3K8 (TPL2, AURA2); NCBI Gene IDs: 4217, 1326); mitogen-activated protein kinase kinase kinase kinase 1 (MAP4K1, HPK1; NCBI Gene ID: 11184); mitogen-activated protein kinase kinases (e.g., MAP2K1 (MEK1), MAP2K2 (MEK2), MAP2K7 (MEK7); NCBI Gene IDs: 5604, 5605, 5609); MPL proto-oncogene, thrombopoietin receptor (MPL; NCBI Gene ID: 4352); mucins (e.g., MUC1 (including splice variants thereof (e.g., including MUC1/A, C, D, X, Y, Z and REP)), MUCSAC, MUC16 (CA125); NCBI Gene IDs: 4582, 4586, 94025); MYC proto-oncogene, bHLH transcription factor (MYC; NCBI Gene ID: 4609); myostatin (MSTN, GDF8; NCBI Gene ID: 2660); myristoylated alanine rich protein kinase C substrate (MARCKS; NCBI Gene ID: 4082); natriuretic peptide receptor 3 (NPR3; NCBI Gene ID: 4883); natural killer cell cytotoxicity receptor 3 ligand 1 (NCR3LG1, B7-H6; NCBI Gene ID: 374383); necdin, MAGE family member (NDN; NCBI Gene ID: 4692); nectin cell adhesion molecules (e.g., NECTIN2 (CD112, PVRL2), NECTIN4 (PVRL4); NCBI Gene IDs: 5819, 81607); neural cell adhesion molecule 1 (NCAM1, CD56; NCBI Gene ID: 4684); neuropilins (e.g., NRP1 (CD304, VEGF165R), NRP2 (VEGF165R2); NCBI Gene IDs: 8828, 8829); neurotrophic receptor tyrosine kinases (e.g., NTRK1 (TRKA), NTRK2 (TRKB), NTRK3 (TRKC); NCBI Gene IDs: 4914, 4915, 4916); NFKB activating protein (NKAP; NCBI Gene ID: 79576); NIMA related kinase 9 (NEK9; NCBI Gene ID: 91754); NLR family pyrin domain containing 3 (NLRP3, NALP3; NCBI Gene ID: 114548); notch receptors (e.g., NOTCH1, NOTCH2, NOTCH3, NOTCH4; NCBI Gene IDs: 4851, 4853, 4854, 4855); NRAS proto-oncogene, GTPase (NRAS; NCBI Gene ID: 4893); nuclear factor kappa B (NFKB1, NFKB2; NCBI Gene IDs: 4790, 4791); nuclear factor, erythroid 2 like 2 (NFE2L2; NRF2; NCBI Gene ID: 4780); nuclear receptor subfamily 4 group A member 1 (NR4A1; NCBI Gene ID: 3164); nucleolin (NCL; NCBI Gene ID: 4691); nucleophosmin 1 (NPM1; NCBI Gene ID: 4869); nucleotide binding oligomerization domain containing 2 (NOD2; NCBI Gene ID: 64127); nudix hydrolase 1 (NUDT1; NCBI Gene ID: 4521); 0-6-methylguanine-DNA methyltransferase (MGMT; NCBI Gene ID: 4255); opioid receptor delta 1 (OPRD1; NCBI Gene ID: 4985); ornithine decarboxylase 1 (ODC1; NCBI Gene ID: 4953); oxoglutarate dehydrogenase (OGDH; NCBI Gene ID: 4967); parathyroid hormone (PTH; NCBI Gene ID: 5741); PD-L1 (CD274; NCBI Gene ID: 29126); periostin (POSTN; NCBI Gene ID: 10631); peroxisome proliferator activated receptors (e.g., PPARA (PPAR alpha), PPARD (PPAR delta), PPARG (PPAR gamma); NCBI Gene IDs: 5465, 5467, 5468); phosphatase and tensin homolog (PTEN; NCBI Gene ID: 5728); phosphatidylinositol-4,5-bisphosphate 3-kinases (PIK3CA (PI3K alpha), PIK3CB (PI3K beta), PIK3CD (PI3K delta), PIK3CG (PI3K gamma); NCBI Gene IDs: 5290, 5291, 5293, 5294); phospholipases (e.g., PLA2G1B, PLA2G2A, PLA2G2D, PLA2G3, PLA2G4A, PLA2G5, PLA2G7, PLA2G10, PLA2G12A, PLA2G12B, PLA2G15; NCBI Gene IDs: 5319, 5320, 5321, 5322, 7941, 8399, 50487, 23659, 26279, 81579, 84647); Pim proto-oncogene, serine/threonine kinases (e.g., PIM1, PIM2, PIM3; NCBI Gene IDs: 5292, 11040, 415116); placenta growth factor (PGF; NCBI Gene ID: 5228); plasminogen activator, urokinase (PLAU, u-PA, ATF; NCBI Gene ID: 5328); platelet derived growth factor receptors (e.g., PDGFRA (CD140A, PDGFR2), FDGFRB (CD140B, PDGFR1); NCBI Gene IDs: 5156, 5159); plexin BI (PLXNB1; NCBI Gene ID: 5364); poliovirus receptor (PVR) cell adhesion molecule (PVR, CD155; NCBI Gene ID: 5817); polo like kinase 1 (PLK1; NCBI Gene ID: 5347); poly(ADP-ribose) polymerases (e.g., PARP1, PARP2, PARP3; NCBI Gene IDs: 142, 10038, 10039); polycomb protein EED (EED; NCBI Gene ID: 8726); porcupine O-acyltransferase (PORCN; NCBI Gene ID: 64840); PRAME nuclear receptor transcriptional regulator (PRAME; NCBI Gene ID: 23532); premelanosome protein (PMEL; NCBI Gene ID: 6490); progesterone receptor (PGR; NCBI Gene ID: 5241); programmed cell death 1 (PDCD1, PD-1, CD279; NCBI Gene ID: 5133); programmed cell death 1 ligand 2 (PDCD1LG2, CD273, PD-L2; NCBI Gene ID: 80380); prominin 1 (PROM1, CD133; NCBI Gene ID: 8842); promyelocytic leukemia (PML; NCBI Gene ID: 5371); prosaposin (PSAP; NCBI Gene ID: 5660); prostaglandin E receptor 4 (PTGER4; NCBI Gene ID: 5734); prostaglandin E synthase (PTGES; NCBI Gene ID: 9536); prostaglandin-endoperoxide synthases (PTGS1 (COX1), PTGS2 (COX2); NCBI Gene ID: 5742, 5743); proteasome 20S subunit beta 9 (PSMB9; NCBI Gene ID: 5698); protein arginine methyltransferases (e.g., PRMT1, PRMT5; NCBI Gene ID: 3276, 10419); protein kinase N3 (PKN3; NCBI Gene ID: 29941); protein phosphatase 2A (PPP2CA; NCBI Gene ID: 5515); protein tyrosine kinase 7 (inactive) (PTK7; NCBI Gene ID: 5754); protein tyrosine phosphatase receptors (PTPRB (PTPB), PTPRC (CD45R); NCBI Gene ID: 5787, 5788); prothymosin alpha (PTMA; NCBI Gene ID: 5757); purine nucleoside phosphorylase (PNP; NCBI Gene ID: 4860); purinergic receptor P2X 7 (P2RX7; NCBI Gene ID: 5027); PVR related immunoglobulin domain containing (PVRIG, CD112R; NCBI Gene ID: 79037); Raf-1 proto-oncogene, serine/threonine kinase (RAF1, c-Raf; NCBI Gene ID: 5894); RAR-related orphan receptor gamma (RORC; NCBI Gene ID: 6097); ras homolog family member C (RHOC); NCBI Gene ID: 389); Ras homolog, mTORC1 binding (RHEB; NCBI Gene ID: 6009); RB transcriptional corepressor 1 (RB1; NCBI Gene ID: 5925); receptor-interacting serine/threonine protein kinase 1 (RIPK1; NCBI Gene ID: 8737); ret proto-oncogene (RET; NCBI Gene ID: 5979); retinoic acid early transcripts (e.g., RAET1E, RAET1G, RAET1L; NCBI Gene IDs: 135250, 154064, 353091); retinoic acid receptors alpha (e.g., RARA, RARG; NCBI Gene IDs: 5914, 5916); retinoid X receptors (e.g., RXRA, RXRB, RXRG; NCBI Gene IDs: 6256, 6257, 6258); Rho associated coiled-coil containing protein kinases (e.g., ROCK1, ROCK2; NCBI Gene IDs: 6093, 9475); ribosomal protein S6 kinase B1 (RPS6KB1, S6K-beta 1; NCBI Gene ID: 6198); ring finger protein 128 (RNF128, GRAIL; NCBI Gene ID: 79589); ROS proto-oncogene 1, receptor tyrosine kinase (ROS1; NCBI Gene ID: 6098); roundabout guidance receptor 4 (ROBO4; NCBI Gene ID: 54538); RUNX family transcription factor 3 (RUNX3; NCBI Gene ID: 864); S100 calcium binding protein A9 (S100A9; NCBI Gene ID: 6280); secreted frizzled related protein 2 (SFRP2; NCBI Gene ID: 6423); secreted phosphoprotein 1 (SPP1; NCBI Gene ID: 6696); secretoglobin family 1A member 1 (SCGB1A1; NCBI Gene ID: 7356); selectins (e.g., SELE, SELL (CD62L), SELP (CD62); NCBI Gene IDs: 6401, 6402, 6403); semaphorin 4D (SEMA4D; CD100; NCBI Gene ID: 10507); sialic acid binding Ig like lectins (SIGLEC7 (CD328), SIGLEC9 (CD329), SIGLEC10; NCBI Gene ID: 27036, 27180, 89790); signal regulatory protein alpha (SIRPA, CD172A; NCBI Gene ID: 140885); signal transducer and activator of transcription (e.g., STAT1, STAT3, STAT5A, STAT5B; NCBI Gene IDs: 6772, 6774, 6776, 6777); sirtuin-3 (SIRT3; NCBI Gene ID: 23410); signaling lymphocytic activation molecule (SLAM) family members (e.g., SLAMFI (CD150), SLAMF6 (CD352), SLAMF7 (CD319), SLAMF8 (CD353), SLAMF9; NCBI Gene IDs: 56833, 57823, 89886, 114836); SLIT and NTRK like family member 6 (SLITRK6; NCBI Gene ID: 84189); smoothened, frizzled class receptor (SMO; NCBI Gene ID: 6608); soluble epoxide hydrolase 2 (EPHX2; NCBI Gene ID: 2053); solute carrier family members (e.g., SLC3A2 (CD98), SLC5A5, SLC6A2, SLC10A3, SLC34A2, SLC39A6, SLC43A2 (LAT4), SLC44A4; NCBI Gene IDs: 6520, 6528, 6530, 8273, 10568, 25800, 80736, 124935); somatostatin receptors (e.g., SSTR1, SSTR2, SSTR3, SSTR4, SSTR5; NCBI Gene IDs: 6751, 6752, 6753, 6754, 6755); sonic hedgehog signaling molecule (SHH; NCBI Gene ID: 6469); Sp1 transcription factor (SP1; NCBI Gene ID: 6667); sphingosine kinases (e.g., SPHK1, SPHK2; NCBI Gene IDs: 8877, 56848); sphingosine-1-phosphate receptor 1 (S1PR1, CD363; NCBI Gene ID: 1901); spleen associated tyrosine kinase (SYK; NCBI Gene ID: 6850); splicing factor 3B factor 1 (SF3B1; NCBI Gene ID: 23451); SRC proto-oncogene, non-receptor tyrosine kinase (SRC; NCBI Gene ID: 6714); stabilin 1 (STAB1, CLEVER-1; NCBI Gene ID: 23166); STEAP family member 1 (STEAP1; NCBI Gene ID: 26872); steroid sulfatase (STS; NCBI Gene ID: 412); stimulator of interferon response cGAMP interactor 1 (STING1; NCBI Gene ID: 340061); superoxide dismutase 1 (SOD1, ALS1; NCBI Gene ID: 6647); suppressors of cytokine signaling (SOCS1 (CISH1), SOCS3 (CISH3); NCBI Gene ID: 8651, 9021); synapsin 3 (SYN3; NCBI Gene ID: 8224); syndecan 1 (SDC1, CD138, syndecan; NCBI Gene ID: 6382); synuclein alpha (SNCA, PARK1; NCBI Gene ID: 6622); T cell immunoglobulin and mucin domain containing 4 (TIMD4, SMUCKLER; NCBI Gene ID: 91937); T cell immunoreceptor with Ig and ITIM domains (TIGIT; NCBI Gene ID: 201633); tachykinin receptors (e.g., TACR1, TACR3; NCBI Gene ID: 6869, 6870); TANK binding kinase 1 (TBK1; NCBI Gene ID: 29110); tankyrase (TNKS; NCBI Gene ID: 8658); TATA-box binding protein associated factor, RNA polymerase I subunit B (TAF1B; NCBI Gene ID: 9014); T-box transcription factor T (TBXT; NCBI Gene ID: 6862); TCDD inducible poly(ADP-ribose) polymerase (TIPARP, PAPR7; NCBI Gene ID: 25976); tec protein tyrosine kinase (TEC; NCBI Gene ID: 7006); TEK receptor tyrosine kinase (TEK, CD202B, TIE2; NCBI Gene ID: 7010); telomerase reverse transcriptase (TERT; NCBI Gene ID: 7015); tenascin C (TNC; NCBI Gene ID: 3371); three prime repair exonucleases (e.g., TREX1, TREX2; NCBI Gene ID: 11277, 11219); thrombomodulin (THBD, CD141; NCBI Gene ID: 7056); thymidine kinases (e.g., TK1, TK2; NCBI Gene IDs: 7083, 7084); thymidine phosphorylase (TYMP; NCBI Gene ID: 1890); thymidylate synthase (TYMS; NCBI Gene ID: 7298); thyroid hormone receptor (THRA, THRB; NCBI Gene IDs: 7606, 7608); thyroid stimulating hormone receptor (TSHR; NCBI Gene ID: 7253); TNF superfamily members (e.g., TNFSF4 (OX40L, CD252), TNFSF5 (CD40L), TNFSF7 (CD70), TNFSF8 (CD153, CD30L), TNFSF9 (4-1BB-L, CD137L), TNFSF10 (TRAIL, CD253, APO2L), TNFSF11 (CD254, RANKL2, TRANCE), TNFSF13 (APRIL, CD256, TRAIL2), TNFSF13b (BAFF, BLYS, CD257), TNFSF14 (CD258, LIGHT), TNFSF18 (GITRL); NCBI Gene IDs: 944, 959, 970, 7292, 8600, 8740, 8741, 8743, 8744, 8995); toll like receptors (e.g., TLR1 (CD281), TLR2 (CD282), TLR3 (CD283), TLR4 (CD284), TLR5, TLR6 (CD286), TLR7, TLR8 (CD288), TLR9 (CD289), TLR10 (CD290); NCBI Gene IDs: 7096, 7097, 7098, 7099, 10333, 51284, 51311, 54106, 81793); transferrin (TF; NCBI Gene ID: 7018); transferrin receptor (TFRC, CD71; NCBI Gene ID: 7037); transforming growth factors (e.g., TGFA, TGFB1; NCBI Gene ID: 7039, 7040); transforming growth factor receptors (e.g., TGFBR1, TGFBR2, TGFBR3; NCBI Gene ID: 7046, 7048, 7049); transforming protein E7 (E7; NCBI Gene ID: 1489079); transglutaminase 5 (TGM5; NCBI Gene ID: 9333); transient receptor potential cation channel subfamily V member 1 (TRPV1, VR1; NCBI Gene ID: 7442); transmembrane and immunoglobulin domain containing 2 (TMIGD2, CD28H, IGPR1; NCBI Gene ID: 126259); triggering receptors expressed on myeloid cells (e.g., TREM1 (CD354), TREM2; NCBI Gene ID: 54209, 54210); trophinin (TRO, MAGED3; NCBI Gene ID: 7216); trophoblast glycoprotein (TPBG; NCBI Gene ID: 7162); tryptophan 2,3-dioxygenase (TDO2; NCBI Gene ID: 6999); tryptophan hydroxylases (e.g., TPH1, TPH2; NCBI Gene ID: 7166, 121278); tumor associated calcium signal transducer 2 (TACSTD2, TROP2, EGP1; NCBI Gene ID: 4070); tumor necrosis factor (TNF; NCBI Gene ID: 7124); tumor necrosis factor (TNF) receptor superfamily members (e.g., TNFRSF1A (CD120a), TNFRSF1B (CD120b), TNFRSF4 (OX40), TNFRSF5 (CD40), TNFRSF6 (CD95, FAS receptor), TNFRSF7 (CD27), TNFRSF8 (CD30), TNFRSF9 (CD137, 4-1BB), TNFRSF10A (CD261), TNFRSF10B (TRAIL, DR5, CD262), TNFRSF1OC, TNFRSF1OD, TNFRSF11A, TNFRSF11B (OPG), TNFRSF12A, TNFRSF13B, TNFR13C (, CD268, BAFFR), TNFRSF14 (CD270, LIGHTR), TNFRSF16, TNFRSF17 (CD269, BCMA), TNFRSF18 (GITR, CD357), TNFRSF19, TNFRSF21, TNFRSF25, NCBI Gene IDs: 355, 608, 939, 943, 958, 3604, 4804, 4982, 7132, 7133, 7293, 8718, 8764, 8784, 8792, 8793, 8794, 8795, 8797, 23495, 27242, 51330, 55504); tumor protein p53 (TP53; NCBI Gene ID: 7157); tumor suppressor 2, mitochondrial calcium regulator (TUSC2; NCBI Gene ID: 11334); TYRO3 protein tyrosine kinase (TYRO3; BYK; NCBI Gene ID: 7301); tyrosinase (TYR; NCBI Gene ID: 7299); tyrosine hydroxylase (TH; NCBI Gene ID: 7054); tyrosine kinase with immunoglobulin like and EGF like domains 1 (e.g., TIE1, TIE1; NCBI Gene ID: 7075); tyrosine-protein phosphatase non-receptor type 11 (PTPN11, SHP2; NCBI Gene ID: 5781); ubiquitin conjugating enzyme E2 I (UBE2I, UBC9; NCBI Gene ID: 7329); ubiquitin C-terminal hydrolase L5 (UCHL5; NCBI Gene ID: 51377); ubiquitin specific peptidase 7 (USP7; NCBI Gene ID: 7874); ubiquitin-like modifier activating enzyme 1 (UBA1; NCBI Gene ID: 7317); UL16 binding proteins (e.g., ULBP1, ULBP2, ULBP3; NCBI Gene ID: 79465, 80328, 80328); valosin-containing protein (VCP, CDC48; NCBI Gene ID: 7415); vascular cell adhesion molecule 1 (VCAM1, CD106; NCBI Gene ID: 7412); vascular endothelial growth factors (e.g., VEGFA, VEGFB; NCBI Gene ID: 7422, 7423); vimentin (VIM; NCBI Gene ID: 7431); vitamin D receptor (VDR; NCBI Gene ID: 7421); V-set domain containing T cell activation inhibitor 1 (VTCN1, B7-H4; NCBI Gene ID: 79679); V-set immunoregulatory receptor (VSIR, VISTA, B7-H5; NCBI Gene ID: 64115); WEE1 G2 checkpoint kinase (WEE1; NCBI Gene ID: 7465); WRN RecQ like helicase (WRN; RECQ3; NCBI Gene ID: 7486); WT1 transcription factor (WT1; NCBI Gene ID: 7490); WW domain containing transcription regulator 1 (WWTR1; TAZ; NCBI Gene ID: 25937); X—C motif chemokine ligand 1 (XCL1, ATAC; NCBI Gene ID: 6375); X—C motif chemokine receptor 1 (XCR1, GPR5, CCXCR1; NCBI Gene ID: 2829); Yes1 associated transcriptional regulator (YAP1; NCBI Gene ID: 10413); zeta chain associated protein kinase 70 (ZAP70; NCBI Gene ID: 7535).

In some embodiments, the one or more additional therapeutic agents include, e.g., an agent targeting 5′-nucleotidase ecto (NT5E or CD73; NCBI Gene ID: 4907); adenosine A2A receptor (ADORA2A; NCBI Gene ID: 135); adenosine A2B receptor (ADORA2B; NCBI Gene ID: 136); C—C motif chemokine receptor 8 (CCR8, CDw198; NCBI Gene ID: 1237); cytokine inducible SH2 containing protein (CISH; NCBI Gene ID: 1154); diacylglycerol kinase alpha (DGKA, DAGK, DAGK1 or DGK-alpha; NCBI Gene ID: 1606); fms like tyrosine kinase 3 (FLT3, CD135; NCBI Gene ID: 2322); integrin associated protein (IAP, CD47; NCBI Gene ID: 961); interleukine-2 (IL2; NCBI Gene ID:3558); interleukine 2 receptor (IL2RA, IL2RB, IL2RG; NCBI Gene IDs: 3559, 3560, 3561); Kirsten rat sarcoma virus (KRAS; NCBI Gene ID: 3845; including mutations, such as KRAS G12C or G12D); mitogen-activated protein kinase kinase kinase kinase 1 (MAP4K1) (also called Hematopoietic Progenitor Kinase 1 (HPK1), NCBI Gene ID: 11184); myeloid cell leukemia sequence 1 apoptosis regulator (MCL1; NCBI Gene ID: 4170); phosphatidylinositol-4,5-bisphosphate 3-kinase, catalytic subunit delta (PIK3CD; NCBI Gene ID: 5293); programmed death-ligand 1 (PD-L1, CD274; NCBI Gene ID 29126); programmed cell death protein 1 (PD-1, CD279; NCBI Gene ID: 5133); proto-oncogen c-KIT (KIT, CD117; NCBI Gene ID: 3815); signal-regulatory protein alpha (SIRPA, CD172A; NCBI Gene ID: 140885); TCDD inducible poly(ADP-ribose) polymerase (TIPARP, PARP7; NCBI Gene ID: 25976); T cell immunoreceptor with Ig and ITIM domains (TIGIT; NCBI Gene ID: 201633); triggering receptor expressed on myeloid cells 1 (TREM1; NCBI Gene ID: 54210); triggering receptor expressed on myeloid cells 2 (TREM2; NCBI Gene ID: 54209); tumor-associated calcium signal transducer 2 (TACSTD2, TROP2, EGP1; NCBI Gene ID: 4070); tumor necrosis factor receptor superfamily, member 4 (TNFRSF4, CD134, OX40; NCBI Gene ID:7293); tumor necrosis factor receptor superfamily, member 9 (TNFRSF9, 4-1BB, CD137; NCBI Gene ID: 3604); tumor necrosis factor receptor superfamily, member 18 (TNFRSF18, CD357, GITR; NCBI Gene ID: 8784); WRN RecQ like helicase (WRN; NCBI Gene ID: 7486); zinc finger protein Helios (IKZF2; NCBI Gene ID: 22807).

### Illustrative Mechanisms of Action

### Immune Checkpoint Modulators

In some embodiments a compound provided herein is administered with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of cancer cells within the tumor microenvironment. Activation or stimulation of stimulatory immune check points can augment the effect of immune checkpoint inhibitors in cancer therapeutics. In some embodiments, the immune checkpoint proteins or receptors regulate T cell responses (e.g., reviewed in Xu, et al., J Exp Clin Cancer Res. (2018) 37:110). In some embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688). Inhibition of regulatory T-cells (Treg) or Treg depletion can alleviate their suppression of antitumor immune responses and have anticancer effects (e.g., reviewed in Plitas and Rudensky, Annu. Rev. Cancer Biol. (2020) 4:459-77; Tanaka and Sakaguchi, Eur. J. Immunol. (2019) 49:1140-1146).

Examples of immune checkpoint proteins or receptors include CD27 (NCBI Gene ID: 939), CD70 (NCBI Gene ID: 970); CD40 (NCBI Gene ID: 958), CD40LG (NCBI Gene ID: 959); CD47 (NCBI Gene ID: 961), SIRPA (NCBI Gene ID: 140885); CD48 (SLAMF2; NCBI Gene ID: 962), transmembrane and immunoglobulin domain containing 2 (TMIGD2, CD28H; NCBI Gene ID: 126259), CD84 (LY9B, SLAMF5; NCBI Gene ID: 8832), CD96 (NCBI Gene ID: 10225), CD160 (NCBI Gene ID: 11126), MS4A1 (CD20; NCBI Gene ID: 931), CD244 (SLAMF4; NCBI Gene ID: 51744); CD276 (B7H3; NCBI Gene ID: 80381); V-set domain containing T cell activation inhibitor 1 (VTCN1, B7H4); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943), TNFSF8 (CD30L; NCBI Gene ID: 944); TNFRSF10A (CD261, DR4, TRAILR1; NCBI Gene ID: 8797), TNFRSF9 (CD137; NCBI Gene ID: 3604), TNFSF9 (CD137L; NCBI Gene ID: 8744); TNFRSF10B (CD262, DR5, TRAILR2; NCBI Gene ID: 8795), TNFRSF10 (TRAIL; NCBI Gene ID: 8743); TNFRSF14 (HVEM, CD270; NCBI Gene ID: 8764), TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608), TNFSF13B (BAFF; NCBI Gene ID: 10673); TNFRSF18 (GITR; NCBI Gene ID: 8784), TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; NCBI Gene ID: 5133); cytotoxic T-lymphocyte associated protein 4 (CTLA4, CD152; NCBI Gene ID: 1493); CD80 (B7-1; NCBI Gene ID: 941), CD28 (NCBI Gene ID: 940); nectin cell adhesion molecule 2 (NECTIN2, CD112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; NCBI Gene ID: 84868); galectin 9 (LGALS9; NCBI Gene ID: 3965); lymphocyte activating 3 (LAG3, CD223; NCBI Gene ID: 3902); signaling lymphocytic activation molecule family member 1 (SLAMF1, SLAM, CD150; NCBI Gene ID: 6504); lymphocyte antigen 9 (LY9, CD229, SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL16 binding protein 1 (ULBP1; NCBI Gene ID: 80329); UL16 binding protein 2 (ULBP2; NCBI Gene ID: 80328); UL16 binding protein 3 (ULBP3; NCBI Gene ID: 79465); retinoic acid early transcript 1E (RAET1E; ULBP4; NCBI Gene ID: 135250); retinoic acid early transcript 1G (RAET1G; ULBP5; NCBI Gene ID: 353091); retinoic acid early transcript 1L (RAET1L; ULBP6; NCBI Gene ID: 154064); killer cell immunoglobulin like receptor, three Ig domains and long cytoplasmic tail 1 (KIR, CD158E1; NCBI Gene ID: 3811, e.g., lirilumab (IPH-2102, IPH-4102)); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1); killer cell lectin like receptor D1 (KLRD1; NCBI Gene ID: 3824); killer cell lectin like receptor G1 (KLRG1; CLEC15A, MAFA, 2F1; NCBI Gene ID: 10219); sialic acid binding Ig like lectin 7 (SIGLEC7; NCBI Gene ID: 27036); and sialic acid binding Ig like lectin 9 (SIGLEC9; NCBI Gene ID: 27180).

In some embodiments a compound provided herein is administered with one or more blockers or inhibitors of one or more T-cell inhibitory immune checkpoint proteins or receptors. Illustrative T-cell inhibitory immune checkpoint proteins or receptors include CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the compound provided herein is administered with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In some embodiments the compound provided herein is administered with one or more blockers or inhibitors of one or more NK-cell inhibitory immune checkpoint proteins or receptors. Illustrative NK-cell inhibitory immune checkpoint proteins or receptors include killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CDT59A); killer cell lectin like receptor D1 (KLRD1, CD94), killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In some embodiments the compound provided herein is administered with one or more agonist or activators of one or more NK-cell stimulatory immune checkpoint proteins or receptors. Illustrative NK-cell stimulatory immune checkpoint proteins or receptors include CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments the one or more immune checkpoint inhibitors comprises a proteinaceous (e.g., antibody or fragment thereof, or antibody mimetic) inhibitor of PD-L1 (CD274), PD-1 (PDCD1), CTLA4, or TIGIT. In some embodiments the one or more immune checkpoint inhibitors comprises a small organic molecule inhibitor of PD-L1 (CD274), PD-1 (PDCD1), CTLA4, or TIGIT. In some embodiments the one or more immune checkpoint inhibitors comprises a proteinaceous (e.g., antibody or fragment thereof, or antibody mimetic) inhibitor of LAG3.

Examples of inhibitors of CTLA4 that can be co-administered include ipilimumab, tremelimumab, BMS-986218, AGEN1181, zalifrelimab (AGEN1884), BMS-986249, MK-1308, REGN-4659, ADU-1604, CS-1002 (ipilimumab biosimilar), BCD-145, APL-509, JS-007, BA-3071, ONC-392, AGEN-2041, HBM-4003, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include pembrolizumab, nivolumab, cemiplimab, pidilizumab, AMP-224, MEDI0680 (AMP-514), spartalizumab, atezolizumab, avelumab, durvalumab, BMS-936559, cosibelimab (CK-301), sasanlimab (PF-06801591), tislelizumab (BGB-A317), GLS-010 (WBP-3055), AK-103 (HX-008), AK-105, CS-1003, HLX-10, retifanlimab (MGA-012), BI-754091, balstilimab (AGEN-2034), AMG-404, toripalimab (JS-001), cetrelimab (JNJ-63723283), genolimzumab (CBT-501), LZM-009, prolgolimab (BCD-100), lodapolimab (LY-3300054), SHR-1201, camrelizumab (SHR-1210), Sym-021, budigalimab (ABBV-181), PD1-PIK, BAT-1306, avelumab (MSB0010718C), CX-072, CBT-502, dostarlimab (TSR-042), MSB-2311, JTX-4014, BGB-A333, SHR-1316, CS-1001 (WBP-3155, envafolimab (KN-035), sintilimab (IBI-308), HLX-20, KL-A167, STI-A1014, STI-A1015 (IMC-001), BCD-135, FAZ-053, TQB-2450, MDX1105-01, GS-4224, GS-4416, INCB086550, MAX10181, zimberelimab (AB122), spartalizumab (PDR-001), and compounds disclosed in WO2018195321, WO2020014643, WO2019160882, or WO2018195321, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1), RO-7247669 (PD-1/LAG-3), MGD-019 (PD-1/CTLA4), KN-046 (PD-1/CTLA4), MEDI-5752 (CTLA4/PD-1), RO-7121661 (PD-1/TIM-3), RG7769 (PD-1/TIM-3), TAK-252 (PD-1/OX40L), XmAb-20717 (PD-1/CTLA4), AK-104 (CTLA4/PD-1), FS-118 (LAG-3/PD-L1), FPT-155 (CTLA4/PD-L1/CD28), GEN-1046 (PD-L1/4-1BB), bintrafusp alpha (M7824; PD-L1/TGFβ-EC domain), CA-170 (PD-L1/VISTA), CDX-527 (CD27/PD-L1), LY-3415244 (TIM3/PDL1), and INBRX-105 (4-1BB/PDL1). In some embodiments the PD-L1 inhibitor is a small molecule inhibitor, such as CA-170, GS-4224, GS-4416 and lazertinib (GNS-1480; PD-L1/EGFR).

Examples of inhibitors of TIGIT that can be co-administered include tiragolumab (RG-6058), vibostolimab, domvanalimab, domvanalimab (AB154), AB308, BMS-986207, AGEN-1307, COM-902, or etigilimab.

Examples of inhibitors of LAG3 that can be co-administered include leramilimab (LAG525).

Inhibition of regulatory T-cell (Treg) activity or Treg depletion can alleviate their suppression of antitumor immune responses and have anticancer effects. See, e.g., Plitas and Rudensky, Annu. Rev. Cancer Biol. (2020) 4:459-77; Tanaka and Sakaguchi, Eur. J. Immunol. (2019) 49:1140-1146. In some embodiments, an compound provided herein is administered with one or more inhibitors of Treg activity or a Treg depleting agent. Treg inhibition or depletion can augment the effect of immune checkpoint inhibitors in cancer therapeutics.

In some embodiments an compound provided herein is administered with one or more Treg inhibitors. In some embodiments the Treg inhibitor can suppress the migration of Tregs into the tumor microenvironment. In some embodiments Treg inhibitor can reduce the immunosuppressive function of Tregs. In some embodiments, the Treg inhibitor can modulate the cellular phenotype and induce production of proinflammatory cytokines. Exemplary Treg inhibitors include without limitation, CCR4 (NCBI Gene ID: 1233) antagonists and degraders of Ikaros zinc-finger proteins (e.g., Ikaros (IKZF1; NCBI Gene ID: 10320), Helios (IKZF2; NCBI Gene ID: 22807), Aiolos (IKZF3; NCBI Gene ID: 22806), and Eos (IKZF4; NCBI Gene ID: 64375).

Examples of Helios degraders that can be co-administered include without limitation I-57 (Novartis) and compounds disclosed in WO2019038717, WO2020012334, WO20200117759, and WO2021101919.

In some embodiments an compound provided herein is administered with one or more Treg depleting agents. In some embodiments the Treg depleting agent is an antibody. In some embodiments the Treg depleting antibody has antibody-dependent cytotoxic (ADCC) activity. In some embodiments, the Treg depleting antibody is Fc-engineered to possess an enhanced ADCC activity. In some embodiments the Treg depleting antibody is an antibody-drug conjugate (ADC). Illustrative targets for Treg depleting agents include without limitation CD25 (IL2RA; NCBI Gene ID: 3559), CTLA4 (CD152; NCBI Gene ID: 1493); GITR (TNFRSF18; NCBI Gene ID: 8784); 4-1BB (CD137; NCBI Gene ID: 3604), OX-40 (CD134; NCBI Gene ID: 7293), LAG3 (CD223; NCBI Gene ID: 3902), TIGIT (NCBI Gene ID: 201633), CCR4 (NCBI Gene ID: 1233), and CCR8 (NCBI Gene ID: 1237).

In some embodiments the Treg inhibitor or Treg depleting agent that can be co-administered comprises an antibody or antigen-binding fragment thereof that selectively binds to a cell surface receptor selected from the group consisting of C—C motif chemokine receptor 4 (CCR4), C—C motif chemokine receptor 7 (CCR7), C—C motif chemokine receptor 8 (CCR8), C—X—C motif chemokine receptor 4 (CXCR4; CD184), TNFRSF4 (OX40), TNFRSF18 (GITR, CD357), TNFRSF9 (4-1BB, CD137), cytotoxic T-lymphocyte associated protein 4 (CTLA4, CD152), programmed cell death 1 (PDCD1, PD-1), Sialyl Lewis x (CD15s), CD27, ectonucleoside triphosphate diphosphohydrolase 1 (ENTPD1; CD39), protein tyrosine phosphatase receptor type C (PTPRC; CD45), neural cell adhesion molecule 1 (NCAM1; CD56), selectin L (SELL; CD62L), integrin subunit alpha E (ITGAE; CD103), interleukin 7 receptor (IL7R; CD127), CD40 ligand (CD40LG; CD154), folate receptor alpha (FOLR1), folate receptor beta (FOLR2), leucine rich repeat containing 32 (LRRC32; GARP), IKAROS family zinc finger 2 (IKZF2; HELIOS), inducible T cell costimulatory (ICOS; CD278), lymphocyte activating 3 (LAG3; CD223), transforming growth factor beta 1 (TGFB1), hepatitis A virus cellular receptor 2 (HAVCR2; CD366; TIM3), T cell immunoreceptor with Ig and ITIM domains (TIGIT), TNF receptor superfamily member 1B (CD120b; TNFR2), IL2R^{A} (CD25) or a combination thereof.

Examples of Treg depleting anti-CCR8 antibodies that can be administered include without limitation JTX-1811 (GS-1811) (Jounce Therapeutics, Gilead Sciences), BMS-986340 (Bristol Meyers Squibb), S-531011 (Shionogi), FPA157 (Five Prime Therapeutics), SRF-114 (Surface Oncology), HBM1022 (Harbor BioMed), IO-1 (Oncurious), and antibodies disclosed in WO2021163064, WO2020138489, and WO2021152186.

Examples of Treg depleting anti-CCR4 antibodies that can be administered include mogamulizumab.

Inhibiting, depleting, or reprogramming of non-stimulatory myeloid cells in the tumor microenvironment can enhance anti-cancer immune responses (see, e.g., Binnewies et al., Nat. Med. (2018) 24(5): 541-550; WO2016049641). Illustrative targets for depleting or reprogramming non-stimulatory myeloid cells include triggering receptors expressed on myeloid cells, TREM-1 (CD354, NCBI Gene ID: 54210) and TREM-2 (NCBI Gene ID: 54209). In some embodiments an compound provided herein is administered with one or more myeloid cell depleting or reprogramming agents, such as an anti-TREM-1 antibody (e.g. PY159; antibodies disclosed in WO2019032624) or an anti-TREM-2 antibody (e.g., PY314; antibodies disclosed in WO2019118513).

### Cluster of Differentiation Agonists or Activators

In some embodiments, the compound provided herein is administered with agents targeting a cluster of differentiation (CD) marker. Exemplary CD marker targeting agents that can be co-administered include without limitation A6, AD-IL24, neratinib, tucatinib (ONT 380), mobocertinib (TAK-788), tesevatinib, trastuzumab (HERCEPTIN®), trastuzumab biosimimar (HLX-02), margetuximab, BAT-8001, pertuzumab (Perjeta), pegfilgrastim, RG6264, zanidatamab (ZW25), cavatak, AIC-100, tagraxofusp (SL-401), HLA-A2402/HLA-A0201 restricted epitope peptide vaccine, dasatinib, imatinib, nilotinib, sorafenib, lenvatinib mesylate, ofranergene obadenovec, cabozantinib malate, AL-8326, ZLJ-33, KBP-7018, sunitinib malate, pazopanib derivatives, AGX-73, rebastinib, NMS-088, lucitanib hydrochloride, midostaurin, cediranib, dovitinib, sitravatinib, tivozanib, masitinib, regorafenib, olverembatinib dimesylate (HQP-1351), cabozantinib, ponatinib, and famitinib L-malate, CX-2029 (ABBV-2029), SCB-313, CA-170, COM-701, CDX-301, GS-3583, asunercept (APG-101), APO-010, and compounds disclosed in WO2016196388, WO2016033570, WO2015157386, WO199203459, WO199221766, WO2004080462, WO2005020921, WO2006009755, WO2007078034, WO2007092403, WO2007127317, WO2008005877, WO2012154480, WO2014100620, WO2014039714, WO2015134536, WO2017167182, WO2018112136, WO2018112140, WO2019155067, WO2020076105, PCT/US2019/063091, WO19173692, WO2016179517, WO2017096179, WO2017096182, WO2017096281, WO2018089628, WO2017096179, WO2018089628, WO2018195321, WO2020014643, WO2019160882, WO2018195321, WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170, WO2020068752, Cancer Discov. 2019 Jan. 9 (1):8; and Gariepy J., et al. 106th Annu Meet Am Assoc Immunologists (AAI) (May 9-13, San Diego, 2019, Abst 71.5).

In some embodiments the CD marker targeting agent that can be co-administered include small molecule inhibitors, such as PBF-1662, BLZ-945, pemigatinib (INCB-054828), rogaratinib (BAY-1163877), AZD4547, roblitinib (FGF-401), quizartinib dihydrochloride, SX-682, AZD-5069, PLX-9486, avapritinib (BLU-285), ripretinib (DCC-2618), imatinib mesylate, JSP-191, BLU-263, CD117-ADC, AZD3229, telatinib, vorolanib, GO-203-2C, AB-680, PSB-12379, PSB-12441, PSB-12425, CB-708, HM-30181A, motixafortide (BL-8040), LY2510924, burixafor (TG-0054), X4P-002, mavorixafor (X4P-001-IO), plerixafor, CTX-5861, or REGN-5678 (PSMA/CD28).

In some embodiments the CD marker targeting agent that can be co-administered include small molecule agonists, such as interleukin 2 receptor subunit gamma, eltrombopag, rintatolimod, poly-ICLC (NSC-301463), Riboxxon, Apoxxim, RIBOXXIM®, MCT-465, MCT-475, G100, PEPA-10, eftozanermin alfa (ABBV-621), E-6887, motolimod, resiquimod, selgantolimod (GS-9688), VTX-1463, NKTR-262, AST-008, CMP-001, cobitolimod, tilsotolimod, litenimod, MGN-1601, BB-006, IMO-8400, IMO-9200, agatolimod, DIMS-9054, DV-1079, lefitolimod (MGN-1703), CYT-003, and PUL-042.

In some embodiments the CD marker targeting agent that can be co-administered include antibodies, such as tafasitamab (MOR208; MorphoSys AG), Inebilizumab (MEDI-551), obinutuzumab, IGN-002, rituximab biosimilar (PF-05280586), varlilumab (CDX-1127), AFM-13 (CD16/CD30), AMG330, otlertuzumab (TRU-016), isatuximab, felzartamab (MOR-202), TAK-079, TAK573, daratumumab (DARZALEX®), TTX-030, selicrelumab (RG7876), APX-005M, ABBV-428, ABBV-927, mitazalimab (JNJ-64457107), lenziluma, alemtuzuma, emactuzumab, AMG-820, FPA-008 (cabiralizumab), PRS-343 (CD-137/Her2), AFM-13 (CD16/CD30), belantamab mafodotin (GSK-2857916), AFM26 (BCMA/CD16A), simlukafusp alfa (RG7461), urelumab, utomilumab (PF-05082566), AGEN2373, ADG-106, BT-7480, PRS-343 (CD-137/HER2), FAP-4-IBBL (4-1BB/FAP), ramucirumab, CDX-0158, CDX-0159 and FSI-174, relatlimab (ONO-4482), LAG-525, MK-4280, fianlimab (REGN-3767), INCAGN2385, encelimab (TSR-033), atipotuzumab, BrevaRex (Mab-AR-20.5), MEDI-9447 (oleclumab), CPX-006, IPH-53, BMS-986179, NZV-930, CPI-006, PAT-SCi, lirilumab (IPH-2102), lacutamab (IPH-4102), monalizumab, BAY-1834942, NEO-201 (CEACAM 5/6), Iodine (1311) apamistamab (131I-BC8 (lomab-B)), MEDI0562 (tavolixizumab), GSK-3174998, INCAGN1949, BMS-986178, GBR-8383, ABBV-368, denosumab, BION-1301, MK-4166, INCAGN-1876, TRX-518, BMS-986156, MK-1248, GWN-323, CTB-006, INBRX-109, GEN-1029, pepinemab (VX-15), vopratelimab (JTX-2011), GSK3359609, cobolimab (TSR-022), MBG-453, INCAGN-2390, and compounds disclosed in WO 2017096179, WO2017096276, WO2017096189, and WO2018089628.

In some embodiments the CD marker targeting agent that can be co-administered include cell therapies, such as CD19-ARTEMIS, TBI-1501, CTL-119 huCART-19 T cells, 1 iso-cel, lisocabtagene maraleucel (JCAR-017), axicabtagene ciloleucel (KTE-C19, Yescarta®), axicabtagene ciloleucel (KTE-X19), U.S. Pat. Nos. 7,741,465, 6,319,494, UCART-19, tabelecleucel (EBV-CTL), T tisagenlecleucel-T (CTL019), CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110, anti-CD19 CAR T-cell therapy (B-cell acute lymphoblastic leukemia, Universiti Kebangsaan Malaysia), anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem, UCAR-011, ICTCAR-014, GC-007F, PTG-01, CC-97540, GC-007G, TC-310, GC-197, tisagenlecleucel-T, CART-19, tisagenlecleucel (CTL-019)), anti-CD20 CAR T-cell therapy (non-Hodgkin's lymphoma), MB-CART2019.1 (CD19/CD20), WZTL-002 dual anti-CD19/anti-CD20 CAR-T cells, ICG-132 (CD19/CD20), ACTR707 ATTCK-20, PBCAR-20A, LB-1905, CIK-CAR.CD33, CD33CART, dual anti-BCMA/anti-CD38 CAR T-cell therapy, CART-ddBCMA, MB-102, IM-23, JEZ-567, UCART-123, PD-1 knockout T cell therapy (esophageal cancer/NSCLC), ICTCAR-052, Tn MUC-1 CAR-T, ICTCAR-053, PD-1 knockout T cell therapy (esophageal cancer/NSCLC), AUTO-2, anti-BCMA CAR T-cell therapy, Descartes-011, anti-BCMA/anti-CD38 CAR T-cell therapy, CART-ddBCMA, BCMA-CSi cCAR, CYAD-01 (NKG2D LIGAND MODULATOR), KD-045, PD-L1 t-haNK, BCMA-CSi cCAR, MEDI5083, anti-CD276 CART, and therapies disclosed in WO2012079000 or WO2017049166.

### Cluster of Differentiation 47 (CD47) Inhibitors

In some embodiments, the compound provided herein is administered with an inhibitor of CD47 (IAP, MER6, OA3; NCBI Gene ID: 961). Examples of CD47 inhibitors include anti-CD47 mAbs (Vx-1004), anti-human CD47 mAbs (CNTO-7108), CC-90002, CC-90002-ST-001, humanized anti-CD47 antibody or a CD47-blocking agent, NI-1701, NI-1801, RCT-1938, ALX148, SG-404, SRF-231, and TTI-621. Additional exemplary anti-CD47 antibodies include CC-90002, magrolimab (Hu5F9-G4), AO-176 (Vx-1004), letaplimab (IBI-188) (letaplimab), lemzoparlimab (TJC-4), SHR-1603, HLX-24, LQ-001, IMC-002, ZL-1201, IMM-01, B6H12, GenSci-059, TAY-018, PT-240, 1F8-GMCSF, SY-102, KD-015, ALX-148, AK-117, TTI-621, TTI-622, or compounds disclosed in WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188, and WO2020009725. In some embodiments, the CD47 inhibitor is RRx-001, DSP-107, VT-1021, IMM-02, SGN-CD47M, or SIRPa-Fc-CD40L (SL-172154). In some embodiments the CD47 inhibitor is magrolimab.

In some embodiments, the CD47 inhibitor is a bispecific antibodies targeting CD47, such as IBI-322 (CD47/PD-L1), IMM-0306 (CD47/CD20), TJ-L1C4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-03 (CD47/CD20), IMM-2502 (CD47/PD-L1), HMBD-004B (CD47/BCMA), HMBD-004A (CD47/CD33), TG-1801 (NI-1701), or NI-1801.

### SIRPa Targeting Agents

In some embodiments the compound provided herein is administered with a SIRPa targeting agent (NCBI Gene ID: 140885; UniProt P78324). Examples of SIRPa targeting agents include SIRPa inhibitors, such as AL-008, RRx-001, and CTX-5861, and anti-SIRPa antibodies, such as FSI-189 (GS-0189), ES-004, BI-765063, ADU1805, CC-95251, Q-1801 (SIRPa/PD-L1). Additional SIRPα-targeting agents of use are described, for example, in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO2020068752.

### FLT3R Agonists

In some embodiments the compound provided herein is administered with a FLT3R agonist. In some embodiments, the compound provided herein is administered with a FLT3 ligand. In some embodiments, the compound provided herein is administered with a FLT3L-Fc fusion protein, e.g., as described in WO2020263830. In some embodiments the compound provided herein is administered with GS-3583 or CDX-301. In some embodiments the compound provided herein is administered with GS-3583.

### TNF Receptor Superfamily (TNFRSF) Member Agonists or Activators

In some embodiments, the compound provided herein is administered with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include MEDI6469, MEDI6383, tavolixizumab (MEDI0562), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include RG7876, SEA-CD40, APX-005M, and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include urelumab, utomilumab (PF-05082566), AGEN-2373, and ADG-106.

In some embodiments the anti-TNFRSF17 (BCMA) antibody GSK-2857916 is co-administered.

Example anti-TNFRSF18 (GITR) antibodies that can be co-administered include MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

Bi-specific antibodies targeting TNFRSF family members that can be co-administered include PRS-343 (CD-137/HER2), AFM26 (BCMA/CD16A), AFM-13 (CD16/CD30), odronextamab (REGN-1979; CD20/CD3), AMG-420 (BCMA/CD3), INHIBRX-105 (4-1BB/PDL1), FAP-4-IBBL (4-1BB/FAP), plamotamab (XmAb-13676; CD3/CD20), RG-7828 (CD20/CD3), CC-93269 (CD3/BCMA), REGN-5458 (CD3/BCMA), and IMM-0306 (CD47/CD20).

### Bi-Specific T-Cell Engagers

In some embodiments, the compound provided herein is administered with a bi-specific T-cell engager (e.g., not having an Fe) or an anti-CD3 bi-specific antibody (e.g., having an Fe). Illustrative anti-CD3 bi-specific antibodies or BiTEs that can be co-administered include duvortuxizumab (JNJ-64052781; CD19/CD3), AMG-211 (CEA/CD3), AMG-160 (PSMA/CD3), RG7802 (CEA/CD3), ERY-974 (CD3/GPC3), PF-06671008 (Cadherins/CD3), APV0436 (CD123/CD3), flotetuzumab (CD123/CD3), odronextamab (REGN-1979; CD20/CD3), MCLA-117 (CD3/CLEC12A), JNJ-0819 (heme/CD3), JNJ-7564 (CD3/heme), AMG-757 (DLL3-CD3), AMG-330 (CD33/CD3), AMG-420 (BCMA/CD3), AMG-427 (FLT3/CD3), AMG-562 (CD19/CD3), AMG-596 (EGFRvIII/CD3), AMG-673 (CD33/CD3), AMG-701 (BCMA/CD3), AMG-757 (DLL3/CD3), AMG-211 (CEA/CD3), blinatumomab (CD19/CD3), huGD2-BsAb (CD3/GD2), ERY974 (GPC3/CD3), GEMoab (CD3/PSCA), RG6026 (CD20/CD3), RG6194 (HER2/CD3), PF-06863135 (BCMA/CD3), SAR440234 (CD3/CDw123), JNJ-9383 (MGD-015), AMG-424 (CD38/CD3), tidutamab (XmAb-18087 (SSTR2/CD3)), JNJ-63709178 (CD123/CD3), MGD-007 (CD3/gpA33), MGD-009 (CD3/B7H3), IMCgp100 (CD3/gp100), XmAb-14045 (CD123/CD3), XmAb-13676 (CD3/CD20), tidutamab (XmAb-18087; SSTR2/CD3), catumaxomab (CD3/EpCAM), REGN-4018 (MUC16/CD3), mosunetuzumab (RG-7828; CD20/CD3), CC-93269 (CD3/BCMA), REGN-5458 (CD3/BCMA), GRB-1302 (CD3/Erbb2), GRB-1342 (CD38/CD3), GEM-333 (CD3/CD33). As appropriate, the anti-CD3 binding bi-specific molecules may or may not have an Fc. Illustrative bi-specific T-cell engagers that can be co-administered target CD3 and a tumor-associated antigen as described herein, including, e.g., CD19 (e.g., blinatumomab); CD33 (e.g., AMG330); CEA (e.g., MEDI-565); receptor tyrosine kinase-like orphan receptor 1 (ROR1) (Gohil, et al., Oncoimmunology. (2017) May 17; 6(7):e1326437); PD-L1 (Horn, et al., Oncotarget. 2017 Aug. 3; 8(35):57964-57980); and EGFRvIII (Yang, et al., Cancer Lett. 2017 Sep. 10; 403:224-230).

### Bi- and Tri-Specific Natural Killer (NK)-Cell Engagers

In some embodiments, the compound provided herein is administered with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FecγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more tumor-associated antigens as described herein, including, e.g., CD19, CD20, CD22, CD30, CD33, CD123, EGFR, EpCAM, ganglioside GD2, HER2/neu, HLA Class II and FOLR1. BiKEs and TriKEs are described, e.g., in Felices, et al., Methods Mol Biol. (2016) 1441:333-346; Fang, et al., Semin Immunol. (2017) 31:37-54.

### MCL1 Apoptosis Regulator, BCL2 Family Member (MCL1) Inhibitors

In some embodiments, the compound provided herein is administered with an inhibitor of MCL1 apoptosis regulator, BCL2 family member (MCL1, TM; EAT; MCL1L; MCL1S; MeI-1; BCL2L3; MCL1-ES; bcl2-L-3; mc11/EAT; NCBI Gene ID: 4170). Examples of MCL1 inhibitors include tapotoclax (AMG-176), AMG-397, S-64315, AZD-5991, 483-LM, A-1210477, UMI-77, JKY-5-037, PRT-1419, GS-9716, and those described in WO2018183418, WO2016033486, and WO2017147410.

### SHP2 Inhibitors

In some embodiments, compound provided herein is administered with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, and those described in WO2018172984 and WO2017211303.

### Hematopoietic Progenitor Kinase 1 (HPK1) Inhibitors and Degraders

In some embodiments, the compound provided herein is administered with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO2020092621, WO2018183956, WO2018183964, WO2018167147, WO2018049152, WO2020092528, WO2016205942, WO2016090300, WO2018049214, WO2018049200, WO2018049191, WO2018102366, WO2018049152, and WO2016090300.

### Apoptosis Signal-Regulating Kinase (ASK) Inhibitors

In some embodiments, the compound provided herein is administered with an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include those described in WO2011008709 (Gilead Sciences) and WO 2013112741 (Gilead Sciences).

### Bruton Tyrosine Kinase (BTK) Inhibitors

In some embodiments, the compound provided herein is administered with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include (S)-6-amino-9-(1-(but-2-ynoyl)pyrrolidin-3-yl)-7-(4-phenoxyphenyl)-7H-purin-8(9H)-one, acalabrutinib (ACP-196), zanubrutinib (BGB-3111), CB988, HM71224, ibrutinib, M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, PCI-32765, and TAS-5315.

### Cyclin-Dependent Kinase (CDK) Inhibitors

In some embodiments, the compound provided herein is administered with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3, NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022), or cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include abemaciclib, alvocidib (HMR-1275, flavopiridol), AT-7519, dinaciclib, ibrance, FLX-925, LEE001, palbociclib, samuraciclib, ribociclib, rigosertib, selinexor, UCN-01, SY1365, CT-7001, SY-1365, G1T38, milciclib, trilaciclib, simurosertib hydrate (TAK931), and TG-02.

### Discoidin Domain Receptor (DDR) Inhibitors

In some embodiments, the compound provided herein is combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Targeted E3 Ligase Ligand Conjugates

In some embodiments, the compound provided herein is administered with a targeted E3 ligase ligand conjugate. Such conjugates have a target protein binding moiety and an E3 ligase binding moiety (e.g., an inhibitor of apoptosis protein (IAP) (e.g., XIAP, c-IAP1, c-IAP2, NIL-IAP, Bruce, and surviving) E3 ubiquitin ligase binding moiety, Von Hippel-Lindau E3 ubiquitin ligase (VHL) binding moiety, a cereblon E3 ubiquitin ligase binding moiety, mouse double minute 2 homolog (MDM2) E3 ubiquitin ligase binding moiety), and can be used to promote or increase the degradation of targeted proteins, e.g., via the ubiquitin pathway. In some embodiments the targeted E3 ligase ligand conjugates comprise a targeting or binding moiety that targets or binds a protein described herein, and an E3 ligase ligand or binding moiety. In some embodiments the targeted E3 ligase ligand conjugates comprise a targeting or binding moiety that targets or binds a protein selected from Cb1 proto-oncogene B (CBLB; Cb1-b, Nb1a00127, RNF56; NCBI Gene ID: 868) and hypoxia inducible factor 1 subunit alpha (HIF1A; NCBI Gene ID: 3091). In some embodiments the targeted E3 ligase ligand conjugates comprise a kinase inhibitor (e.g., a small molecule kinase inhibitor, e.g., of BTK and an E3 ligase ligand or binding moiety. See, e.g., WO2018098280. In some embodiments the targeted E3 ligase ligand conjugates comprise a binding moiety targeting or binding to Interleukin-1 (IL-1) Receptor-Associated Kinase-4 (IRAK-4); Rapidly Accelerated Fibrosarcoma (RAF, such as c-RAF, A-RAF and/or B-RAF), c-Met/p38, or a BRD protein; and an E3 ligase ligand or binding moiety. See, e.g., WO2019099926, WO2018226542, WO2018119448, WO2018223909, WO2019079701. Additional targeted E3 ligase ligand conjugates that can be co-administered are described, e.g., in WO2018237026, WO2019084026, WO2019084030, WO2019067733, WO2019043217, WO2019043208, and WO2018144649.

### Histone Deacetylase (HDAC) Inhibitors

In some embodiments, the compound provided herein is administered with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, and entinostat.

### Indoleamine-pyrrole-2,3-dioxygenase (IDO1) Inhibitors

In some embodiments the compound provided herein is administered with an inhibitor of indoleamine 2,3-dioxygenase 1 (IDO1; NCBI Gene ID: 3620). Examples of IDO1 inhibitors include BLV-0801, epacadostat, linrodostat (F-001287, BMS-986205), GBV-1012, GBV-1028, GDC-0919, indoximod, NKTR-218, NLG-919-based vaccine, PF-06840003, pyranonaphthoquinone derivatives (SN-35837), resminostat, SBLK-200802, and shIDO-ST, EOS-200271, KHK-2455, and LY-3381916.

### Janus Kinase (JAK) Inhibitors

In some embodiments, the compound provided herein is administered with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718). Examples of JAK inhibitors include AT9283, AZD1480, baricitinib, BMS-911543, fedratinib, filgotinib (GLPG0634), gandotinib (LY2784544), INCB039110 (itacitinib), lestaurtinib, momelotinib (CYT0387), ilginatinib maleate (NS-018), pacritinib (SB1518), peficitinib (ASP015K), ruxolitinib, tofacitinib (formerly tasocitinib), INCB052793, and XL019.

### Lysyl Oxidase-Like Protein (LOXL) Inhibitors

In some embodiments the compound provided herein is administered with an inhibitor of a LOXL protein, e.g., LOXL1 (NCBI Gene ID: 4016), LOXL2 (NCBI Gene ID: 4017), LOXL3 (NCBI Gene ID: 84695), LOXL4 (NCBI Gene ID: 84171), and/or LOX (NCBI Gene ID: 4015). Examples of LOXL2 inhibitors include the antibodies described in WO 2009017833 (Arresto Biosciences), WO 2009035791 (Arresto Biosciences), and WO 2011097513 (Gilead Biologics).

### Matrix Metalloprotease (MMP) Inhibitors

In some embodiments the compound provided herein is administered with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148). Examples of MMP9 inhibitors include marimastat (BB-2516), cipemastat (Ro 32-3555), GS-5745 (andecaliximab), and those described in WO 2012027721 (Gilead Biologics).

### RAS and RAS Pathway Inhibitors

In some embodiments the compound provided herein is administered with an inhibitor of KRAS proto-oncogene, GTPase (KRAS; a.k.a., NS; NS3; CFC2; RALD; K-Ras; KRAS1; KRAS2; RASK2; KI-RAS; C-K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893) or HRAS proto-oncogene, GTPase (HRAS; a.k.a., CTLO; KRAS; HAMSV; HRAS1; KRAS2; RASH1; RASK2; Ki-Ras; p2lras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR. Illustrative K-Ras inhibitors that can be co-administered include sotorasib (AMG-510), COTI-219, ARS-3248, WDB-178, BI-3406, BI-1701963, SML-8-73-1 (G12C), adagrasib (MRTX-849), ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 and KRpep-2d. Illustrative KRAS mRNA inhibitors include anti-KRAS U1 adaptor, AZD-4785, siG12D-LODER™, and siG12D exosomes. Illustrative MEK inhibitors that can be co-administered include binimetinib, cobimetinib, PD-0325901, pimasertib, RG-7304, selumetinib, trametinib, and those described below and herein. Illustrative Raf dimer inhibitors that can be co-administered include BGB-283, HM-95573, LXH-254, LY-3009120, RG7304 and TAK-580. Illustrative ERK inhibitors that can be co-administered include LTT-462, LY-3214996, MK-8353, ravoxertinib and ulixertinib. Illustrative Ras GTPase inhibitors that can be co-administered include rigosertib. Illustrative PI3K inhibitors that can be co-administered include idelalisib (Zydelig®), alpelisib, buparlisib, pictilisib, inavolisib (RG6114), ASN-003. Illustrative AKT inhibitors that can be co-administered include capivasertib and GSK2141795. Illustrative PI3K/mTOR inhibitors that can be co-administered include dactolisib, omipalisib, voxtalisib. gedatolisib, GSK2141795, GSK-2126458, inavolisib (RG6114), sapanisertib, ME-344, sirolimus (oral nano-amorphous formulation, cancer), racemetyrosine (TYME-88 (mTOR/cytochrome P450 3A4)), temsirolimus (TORISEL®, CCI-779), CC-115, onatasertib (CC-223), SF-1126, and PQR-309 (bimiralisib). In some embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

### Mitogen-activated Protein Kinase (MEK) Inhibitors

In some embodiments the compound provided herein is administered with an inhibitor of mitogen-activated protein kinase kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, and refametinib.

Phosphatidylinositol 3-kinase (PI3K) Inhibitors

In some embodiments compounds provided herein is administered with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments the PI3K inhibitor is a pan-PI3K inhibitor. Examples of PI3K inhibitors include ACP-319, AEZA-129, AMG-319, AS252424, AZD8186, BAY 10824391, BEZ235, buparlisib (BKM120), BYL719 (alpelisib), CH5132799, copanlisib (BAY 80-6946), duvelisib, GDC-0032, GDC-0077, GDC-0941, GDC-0980, GSK2636771, GSK2269557, idelalisib (Zydelig®), INCB50465, IPI-145, IPI-443, IPI-549, KAR4141, LY294002, LY3023414, MLN1117, OXY111A, PA799, PX-866, RG7604, rigosertib, RP5090, RP6530, SRX3177, taselisib, TG100115, TGR-1202 (umbralisib), TGX221, WX-037, X-339, X-414, XL147 (SAR245408), XL499, XL756, wortmannin, ZSTK474, and the compounds described in WO2005113556 (ICOS), WO 2013/052699 (Gilead Calistoga), WO2013116562 (Gilead Calistoga), WO2014100765 (Gilead Calistoga), WO2014100767 (Gilead Calistoga), and WO2014201409 (Gilead Sciences).

### Spleen Tyrosine Kinase (SYK) Inhibitors

In some embodiments the compound provided herein is administered with an inhibitor of spleen associated tyrosine kinase (SYK, p72-Syk, NCBI Gene ID: 6850). Examples of SYK inhibitors include 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), gusacitinib (ASN-002), and those described in U.S. Pat. No. 8,450,321 (Gilead Connecticut) and US20150175616.

### Toll-Like Receptor (TLR) Agonists

In some embodiments compound provided herein is administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include DS-0509, GS-9620 (vesatolimod), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, BDB-001, DSP-0509, and the compounds disclosed in US20100143301 (Gilead Sciences), US20110098248 (Gilead Sciences), and US20090047249 (Gilead Sciences), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10 and PUL-042. Examples of TLR3 agonist include rintatolimod, poly-ICLC, RIBOXXON®, Apoxxim, RIBOXXIM®, IPH-33, MCT-465, MCT-475, and ND-1.1.

### Tyrosine-Kinase Inhibitors (TKIs)

In some embodiments the compound provided herein is administered with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation afatinib, ARQ-087 (derazantinib), asp5878, AZD3759, AZD4547, bosutinib, brigatinib, cabozantinib, cediranib, crenolanib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, osimertinib (AZD-9291), ponatinib, poziotinib, quizartinib, radotinib, rociletinib, sulfatinib (HMPL-012), sunitinib, famitinib L-malate, (MAC-4), tivoanib, TH-4000, and MEDI-575 (anti-PDGFR antibody). Exemplary EGFR targeting agents include neratinib, tucatinib (ONT-380), tesevatinib, mobocertinib (TAK-788), DZD-9008, varlitinib, abivertinib (ACEA-0010), EGF816 (nazartinib), olmutinib (BI-1482694), osimertinib (AZD-9291), AMG-596 (EGFRvIII/CD3), lifirafenib (BGB-283), vectibix, lazertinib (LECLAZA®), and compounds disclosed in Booth, et al., Cancer Biol Ther. 2018 Feb. 1; 19(2):132-137. Antibodies targeting EGFR include without limitation modotuximab, cetuximab sarotalocan (RM-1929), seribantumab, necitumumab, depatuxizumab mafodotin (ABT-414), tomuzotuximab, depatuxizumab (ABT-806), and cetuximab.

### Chemotherapeutic Agents

In some embodiments the compound provided herein is administered with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer. Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223; trichothecenes, especially T-2 toxin, verracurin A, roridin A, and anguidine; taxoids such as paclitaxel (TAXOL®), abraxane, docetaxel (TAXOTERE®), cabazitaxel, BIND-014, tesetaxel; sabizabulin (Veru-111); platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include apalutamide, abiraterone, enzalutamide, flutamide, galeterone, nilutamide, bicalutamide, leuprolide, goserelin, ODM-201, APC-100, ODM-204, enobosarm (GTX-024), darolutamide, and IONIS-AR-2.5Rx (antisense).

An example progesterone receptor antagonist includes onapristone. Additional progesterone targeting agents include TRI-CYCLEN LO (norethindrone+ethinyl estradiol), norgestimate+ethinylestradiol (Tri-Cyclen) and levonorgestrel.

### Anti-Angiogenic Agents

In some embodiments the compound provided herein is administered with an anti-angiogenic agent. Anti-angiogenic agents that can be co-administered include retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2. Examples for anti-VEGFA antibodies that can be co-administered include bevacizumab, vanucizumab, faricimab, dilpacimab (ABT-165; DLL4/VEGF), or navicixizumab (OMP-305B83; DLL4/VEGF).

### Anti-Fibrotic Agents

In some embodiments the compound provided herein is administered with an anti-fibrotic agent. Anti-fibrotic agents that can be co-administered include the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 20040248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Anti-Inflammatory Agents

In some embodiments the compound provided herein is administered with an anti-inflammatory agent. Example anti-inflammatory agents include without limitation inhibitors of one or more of arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240), soluble epoxide hydrolase 2 (EPHX2, SEH; NCBI Gene ID: 2053) and/or mitogen-activated protein kinase kinase kinase 8 (MAP3K8, TPL2; NCBI Gene ID: 1326). In some embodiments, the inhibitor is a dual inhibitor, e.g., a dual inhibitor of COX-2/COX-1, COX-2/SEH, COX-2/CA, COX-2/5-LOX.

Examples of inhibitors of prostaglandin-endoperoxide synthase 1 (PTGS1, COX-1; NCBI Gene ID: 5742) that can be co-administered include mofezolac, GLY-230, and TRK-700.

Examples of inhibitors of prostaglandin-endoperoxide synthase 2 (PTGS2, COX-2; NCBI Gene ID: 5743) that can be co-administered include diclofenac, meloxicam, parecoxib, etoricoxib, AP-101, celecoxib, AXS-06, diclofenac potassium, DRGT-46, AAT-076, meisuoshuli, lumiracoxib, meloxicam, valdecoxib, zaltoprofen, nimesulide, anitrazafen, apricoxib, cimicoxib, deracoxib, flumizole, firocoxib, mavacoxib, NS-398, pamicogrel, parecoxib, robenacoxib, rofecoxib, rutecarpine, tilmacoxib, and zaltoprofen. Examples of dual COX1/COX2 inhibitors that can be co-administered include HP-5000, lornoxicam, ketorolac tromethamine, bromfenac sodium, ATB-346, HP-5000. Examples of dual COX-2/carbonic anhydrase (CA) inhibitors that can be co-administered include polmacoxib and imrecoxib.

Examples of inhibitors of secreted phospholipase A2, prostaglandin E synthase (PTGES, PGES; Gene ID: 9536) that can be co-administered include LY3023703, GRC 27864, and compounds described in WO2015158204, WO2013024898, WO2006063466, WO2007059610, WO2007124589, WO2010100249, WO2010034796, WO2010034797, WO2012022793, WO2012076673, WO2012076672, WO2010034798, WO2010034799, WO2012022792, WO2009103778, WO2011048004, WO2012087771, WO2012161965, WO2013118071, WO2013072825, WO2014167444, WO2009138376, WO2011023812, WO2012110860, WO2013153535, WO2009130242, WO2009146696, WO2013186692, WO2015059618, WO2016069376, WO2016069374, WO2009117985, WO2009064250, WO2009064251, WO2009082347, WO2009117987, and WO2008071173. Metformin has further been found to repress the COX2/PGE2/STAT3 axis, and can be co-administered. See, e.g., Tong, et al., Cancer Lett. (2017) 389:23-32; and Liu, et al., Oncotarget. (2016) 7(19):28235-46.

Examples of inhibitors of carbonic anhydrase (e.g., one or more of CA1 (NCBI Gene ID: 759), CA2 (NCBI Gene ID: 760), CA3 (NCBI Gene ID: 761), CA4 (NCBI Gene ID: 762), CA5A (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)) that can be co-administered include acetazolamide, methazolamide, dorzolamide, zonisamide, brinzolamide and dichlorphenamide. A dual COX-2/CA1/CA2 inhibitor that can be co-administered includes CG100649.

Examples of inhibitors of arachidonate 5-lipoxygenase (ALOX5, 5-LOX; NCBI Gene ID: 240) that can be co-administered include meclofenamate sodium, zileuton.

Examples of inhibitors of soluble epoxide hydrolase 2 (EPHX2, SEH; NCBI Gene ID: 2053) that can be co-administered include compounds described in WO2015148954. Dual inhibitors of COX-2/SEH that can be co-administered include compounds described in WO2012082647. Dual inhibitors of SEH and fatty acid amide hydrolase (FAAH; NCBI Gene ID: 2166) that can be co-administered include compounds described in WO2017160861.

Examples of inhibitors of mitogen-activated protein kinase kinase kinase 8 (MAP3K8, tumor progression loci-2, TPL2; NCBI Gene ID: 1326) that can be co-administered include GS-4875, GS-5290, BHM-078 and those described in WO2006124944, WO2006124692, WO2014064215, WO2018005435, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42; and Hu, et al., Bioorg Med Chem Lett. (2011) 21(16):4758-61.

### Tumor Oxygenation Agents

In some embodiments the compound provided herein is administered with an agent that promotes or increases tumor oxygenation or reoxygenation, or prevents or reduces tumor hypoxia.

Illustrative agents that can be co-administered include, e.g., Hypoxia inducible factor-1 alpha (HIF-1α) inhibitors, such as PT-2977, PT-2385; VEGF inhibitors, such as bevasizumab, IMC-3C5, GNR-011, tanibirumab, LYN-00101, ABT-165; and/or an oxygen carrier protein (e.g., a heme nitric oxide and/or oxygen binding protein (HNOX)), such as OMX-302 and HNOX proteins described in WO2007137767, WO2007139791, WO2014107171, and WO2016149562.

### Immunotherapeutic Agents

In some embodiments the compound provided herein is administered with an immunotherapeutic agent. In some embodiments the immunotherapeutic agent is an antibody.

Example immunotherapeutic agents that can be co-administered include abagovomab, AB308, ABP-980, adecatumumab, afutuzumab, alemtuzumab, altumomab, amatuximab, anatumomab, arcitumomab, atezolizumab, bavituximab, bectumomab, bevacizumab, bivatuzumab, blinatumomab, brentuximab, camidanlumab, cantuzumab, catumaxomab, CC49, cetuximab, citatuzumab, cixutumumab, clivatuzumab, conatumumab, dacetuzumab, dalotuzumab, daratumumab, detumomab, dinutuximab, domvanalimab, drozitumab, duligotumab, dusigitumab, ecromeximab, elotuzumab, emibetuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, mogamulizumab, moxetumomab, naptumomab, narnatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, tucotuzumab, ubilituximab, veltuzumab, vorsetuzumab, votumumab, zalutumumab, zimberelimab, and 3F8. Rituximab can be used for treating indolent B-cell cancers, including marginal-zone lymphoma, WM, CLL, and small lymphocytic lymphoma. A combination of rituximab and chemotherapy agents is especially effective.

The exemplified therapeutic antibodies can be further labeled or combined with a radioisotope particle such as indium-111, yttrium-90 (90Y-clivatuzumab), or iodine-131.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein. Example ADCs that can be co-administered include gemtuzumab, brentuximab, belantamab (e.g., belantamab mafodotin), camidanlumab (e.g., camidanlumab tesirine), trastuzumab (e.g., trastuzumab deruxtecan; trasuzumab emtansine), inotuzumab, glembatumumab, anetumab, mirvetuximab (e.g., mirvetuximab soravtansine), depatuxizumab, vadastuximab, labetuzumab, ladiratuzumab (e.g., ladiratuzumab vedotin), loncastuximab (e.g., loncastuximab tesirine), sacituzumab (e.g., sacituzumab govitecan), datopotamab (e.g., datopotamab deruxtecan; DS-1062; Dato-DXd), patritumab (e.g., patritumab deruxtecan), lifastuzumab, indusatumab, polatuzumab (e.g., polatuzumab vedotin), pinatuzumab, coltuximab, upifitamab (e.g., upifitamab rilsodotin), indatuximab, milatuzumab, rovalpituzumab (e.g., rovalpituzumab tesirine), enfortumab (e.g., enfortumab vedotin), tisotumab (e.g., tisotumab vedotin), tusamitamab (e.g., tusamitamab ravtansine), disitamab (e.g., disitamab vedotin), telisotuzumab vedotin (ABBV-399), AGS-16C3F, ASG-22ME, AGS67E, AMG172, AMG575, BAY1129980, BAY1187982, BAY94-9343, GSK2857916, Humax-TF-ADC, IMGN289, IMGN151, IMGN529, IMGN632, IMGN853, IMGC936, LOP628, PCA062, MDX-1203 (BMS936561), MEDI-547, PF-06263507, PF-06647020, PF-06647263, PF-06664178, RG7450, RG7458, RG7598, SAR566658, SGN-CD19A, SGN-CD33A, SGN-CD70A, SGN-LIV1A, SYD985, DS-7300, XMT-1660, IMMU-130, and IMMU-140. ADCs that can be co-administered are described, e.g., in Lambert, et al., Adv Ther (2017) 34:1015-1035 and in de Goeij, Current Opinion in Immunology (2016) 40:14-23.

Illustrative therapeutic agents (e.g., anticancer or antineoplastic agents) that can be conjugated to the drug-conjugated antibodies, fragments thereof, or antibody mimetics include without limitation monomethyl auristatin E (MMAE), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., doxorubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, a duocarmycin (A, B1, B2, C1, C2, D, SA, CC-1065), and other anticancer or anti-neoplastic agents described herein. In some embodiments, the therapeutic agent conjugated to the drug-conjugated antibody is a topoisomerase I inhibitor (e.g., a camptothecin analog, such as irinotecan or its active metabolite SN38). In some embodiments, the therapeutic agents (e.g., anticancer or antineoplastic agents) that can be conjugated to the drug-conjugated antibodies, fragments thereof, or antibody mimetics include an immune checkpoint inhibitor. In some embodiments the conjugated immune checkpoint inhibitor is a conjugated small molecule inhibitor of CD274 (PDL1, PD-L1), programmed cell death 1 (PDCD1, PD1, PD-1) or CTLA4. In some embodiments the conjugated small molecule inhibitor of CD274 or PDCD1 is selected from the group consisting of GS-4224, GS-4416, INCB086550 and MAX10181. In some embodiments the conjugated small molecule inhibitor of CTLA4 comprises BPI-002.

In some embodiments the ADCs that can be co-administered include an antibody targeting tumor-associated calcium signal transducer 2 (TROP-2; TACSTD2; EGP-1; NCBI Gene ID: 4070). Illustrative anti-TROP-2 antibodies include without limitation TROP2-XPAT (Amunix), BAT-8003 (Bio-Thera Solutions), TROP-2-IR700 (Chiome Bioscience), datopotamab deruxtecan (Daiichi Sankyo, AstraZeneca), GQ-1003 (Genequantum Healthcare, Samsung BioLogics), DAC-002 (Hangzhou DAC Biotech, Shanghai Junshi Biosciences), sacituzumab govitecan (Gilead Sciences), E1-3s (Immunomedics/Gilead, IBC Pharmaceuticals), TROP2-TRACTr (Janux Therapeutics), LIV-2008 (LivTech/Chiome, Yakult Honsha, Shanghai Henlius BioTech), LIV-2008b (LivTech/Chiome), anti-TROP-2a (Oncoxx), anti-TROP-2b (Oncoxx), OXG-64 (Oncoxx), OXS-55 (Oncoxx), humanized anti-Trop2-SN38 antibody conjugate (Shanghai Escugen Biotechnology, TOT Biopharma), anti-Trop2 antibody-CLB-SN-38 conjugate (Shanghai Fudan-Zhangjiang Bio-Pharmaceutical), SKB-264 (Sichuan Kelun Pharmaceutical/Klus Pharma), TROP2-Ab8 (Abmart), Trop2-IgG (Nanjing Medical University (NMU)), 90Y-DTPA-AF650 (Peking University First Hospital), hRS7-CM (SynAffix), 89Zr-DFO-AF650 (University of Wisconsin-Madison), anti-Trop2 antibody (Mediterranea Theranostic, LegoChem Biosciences), KD-065 (Nanjing KAEDI Biotech), and those described in WO2020016662 (Abmart), WO2020249063 (Bio-Thera Solutions), US20190048095 (Bio-Thera Solutions), WO2013077458 (LivTech/Chiome), EP20110783675 (Chiome), WO2015098099 (Daiichi Sankyo), WO2017002776 (Daiichi Sankyo), WO2020130125 (Daiichi Sankyo), WO2020240467 (Daiichi Sankyo), US2021093730 (Daiichi Sankyo), U.S. Pat. No. 9,850,312 (Daiichi Sankyo), CN112321715 (Biosion), US2006193865 (Immunomedics/Gilead), WO2011068845 (Immunomedics/Gilead), US2016296633 (Immunomedics/Gilead), US2017021017 (Immunomedics/Gilead), US2017209594 (Immunomedics/Gilead), US2017274093 (Immunomedics/Gilead), US2018110772 (Immunomedics/Gilead), US2018185351 (Immunomedics/Gilead), US2018271992 (Immunomedics/Gilead), WO2018217227 (Immunomedics/Gilead), US2019248917 (Immunomedics/Gilead), CN111534585 (Immunomedics/Gilead), US2021093730 (Immunomedics/Gilead), US2021069343 (Immunomedics/Gilead), U.S. Pat. No. 8,435,539 (Immunomedics/Gilead), U.S. Pat. No. 8,435,529 (Immunomedics/Gilead), U.S. Pat. No. 9,492,566 (Immunomedics/Gilead), WO2003074566 (Gilead), WO2020257648 (Gilead), US2013039861 (Gilead), WO2014163684 (Gilead), U.S. Pat. No. 9,427,464 (LivTech/Chiome), U.S. Ser. No. 10/501,555 (Abruzzo Theranostic/Oncoxx), WO2018036428 (Sichuan Kelun Pharma), WO2013068946 (Pfizer), WO2007095749 (Roche), and WO2020094670 (SynAffix). In some embodiments, the anti-Trop-2 antibody is selected from hRS7, Trop-2-XPAT, and BAT-8003. In some embodiments, the anti-Trop-2 antibody is hRS7. In some embodiments, hRS7 is as disclosed in U.S. Pat. Nos. 7,238,785; 7,517,964 and 8,084,583, which are incorporated herein by reference. In some embodiments, the antibody-drug conjugate comprises an anti-Trop-2 antibody and an anticancer agent linked by a linker. In some embodiments, the linker includes the linkers disclosed in U.S. Pat. No. 7,999,083. In some embodiments, the linker is CL2A. In some embodiments, the drug moiety of antibody-drug conjugate is a chemotherapeutic agent. In some embodiments, the chemotherapeutic agent is selected from doxorubcin (DOX), epirubicin, morpholinodoxorubicin (morpholino-DOX), cyanomorpholino-doxorubicin (cyanomorpholino-DOX), 2-pyrrolino-doxorubicin (2-PDOX), CPT, 10-hydroxy camptothecin, SN-38, topotecan, lurtotecan, 9-aminocamptothecin, 9-nitrocamptothecin, taxanes, geldanamycin, ansamycins, and epothilones. In some embodiments, the chemotherapeutic moiety is SN-38. In some embodiments the compound provided herein is administered with sacituzumab govitecan.

In some embodiments the ADCs that can be co-administered include an antibody targeting carcinoembryonic antigen-related cell adhesion molecule 1 (CEACAM1; CD66a; NCBI Gene ID: 634). In some embodiments the CEACAM1 antibody is hMN-14 (e.g., as described in WO1996011013). In some embodiments the CEACAM1-ADC is as described in WO2010093395 (anti-CEACAM-1-CL2A-SN38). In some embodiments the compound provided herein is administered with the CEACAM1-ADC IMMU-130.

In some embodiments the ADCs that can be co-administered include an antibody targeting MHC class II cell surface receptor encoded by the human leukocyte antigen complex (HLA-DR).

In some embodiments the HLA-DR antibody is hL243 (e.g., as described in WO2006094192). In some embodiments the HLA-DR-ADC is as described in WO2010093395 (anti-HLA-DR-CL2A-SN38). In some embodiments the compound provided herein is administered with the HLA-DR-ADC IMMU-140.

### Cancer Gene Therapy and Cell Therapy

In some embodiments the compound provided herein is administered with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In some embodiments the compound provided herein is administered with one or more cellular therapies. Illustrative cellular therapies include without limitation co-administration of one or more of a population of natural killer (NK) cells, NK-T cells, T cells, cytokine-induced killer (CIK) cells, macrophage (MAC) cells, tumor infiltrating lymphocytes (TILs) and/or dendritic cells (DCs). In some embodiments, the cellular therapy entails a T cell therapy, e.g., co-administering a population of alpha/beta TCR T cells, gamma/delta TCR T cells, regulatory T (Treg) cells and/or TRuC™ T cells. In some embodiments, the cellular therapy entails a NK cell therapy, e.g., co-administering NK-92 cells. As appropriate, a cellular therapy can entail the co-administration of cells that are autologous, syngeneic or allogeneic to the subject.

In some embodiments the cellular therapy entails co-administering cells comprising chimeric antigen receptors (CARs). In such therapies, a population of immune effector cells engineered to express a CAR, wherein the CAR comprises a tumor antigen-binding domain. In T cell therapies, the T cell receptors (TCRs) are engineered to target tumor derived peptides presented on the surface of tumor cells.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon R1b), CD79a, CD79b, Fcgamma RIIa, DAP10, and DAP12.

In some embodiments, the costimulatory domain comprises a functional domain of one or more proteins selected from the group consisting of CD27, CD28, 4-1BB(CD137), OX40, CD30, CD40, PD-1, ICOS, CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFI), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD160, CD19, IL2R beta, IL2R gamma, IL7R, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMFI, CD150, IPO-3), BLAME (SLAMF8), SELPLG (CD162), LTBR, PAG/Cbp, NKp44, NKp30, NKp46, NKG2D, and NKG2C.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvlll); ganglioside G2 (GD2); ganglioside GD3 (αNeuSAc(2-8)αNeuSAc(2-3)βDGaip(1-4)bDGIcp(1-1)Cer); ganglioside GM3 (αNeuSAc(2-3)βDGalp(1-4)DDGlcp(1-1)Cer); TNF receptor superfamily member 17 (TNFRSFi7, BCMA); Tn antigen ((Tn Ag) or (GalNAcu-Ser/Thr)); prostate-specific membrane antigen (PSMA); receptor tyrosine kinase-like orphan receptor 1 (RORI); tumor-associated glycoprotein 72 (TAG72); CD38; CD44v6; Carcinoembryonic antigen (CEA); epithelial cell adhesion molecule (EPCAM); B7H3 (CD276); KIT (CD117); interleukin-13 receptor subunit alpha-2 (IL-13Ra2 or CD213A2); mesothelin; interleukin 11 receptor alpha (IL-11Ra); prostate stem cell antigen (PSCA); protease serine 21 (Testisin or PRSS21); vascular endothelial growth factor receptor 2 (VEGFR2); Lewis(Y)antigen; CD24; platelet-derived growth factor receptor beta (PDGFR-beta); stage-specific embryonic antigen-4 (SSEA-4); CD20; delta like 3 (DLL3); folate receptor alpha; receptor tyrosine-protein kinase, ERBB2 (Her2/neu); mucin 1, cell surface associated (MUC1); epidermal growth factor receptor (EGFR); neural cell adhesion molecule (NCAM); prostase; prostatic acid phosphatase (PAP); elongation factor 2 mutated (ELF2M); ephrin B2; fibroblast activation protein alpha (FAP); insulin-like growth factor 1 receptor (IGF-I receptor), carbonic anhydrase IX (CAIX); proteasome (Prosome, Macropain) subunit, beta type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); fucosyl GM1; sialyl Lewis adhesion molecule (sLe); transglutaminase 5 (TGS5); high molecular weight-melanoma associated antigen (HMWMAA); o-acetyl-GD2 ganglioside (OAcGD2); folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); polysialic acid; placenta-specific 1 (PLAC1); hexasaccharide portion of globoH glycoceramide (GloboH); mammary gland differentiation antigen (NY-BR-1); uroplakin 2 (UPK2); hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); olfactory receptor 51E2 (ORS IE2); TCR Gamma Alternate Reading Frame Protein (TARP); Wilms tumor protein (WTi); cancer/testis antigen 1 (NY-ESO-1); cancer/testis antigen 2 (LAGE-1a); melanoma associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); rat sarcoma (Ras) mutant; human telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); androgen receptor; cyclin B1; v-myc avian myelocytomatosis viral oncogene neuroblastoma derived homolog (MYCN); ras homolog family member C (RhoC); tyrosinase-related protein 2 (TRP-2); cytochrome P450 1B1(CYP IBI); CCCTC-Binding Factor (Zinc Finger Protein)-Like (BORIS or Brother of the Regulator of Imprinted Sites), squamous cell carcinoma antigen recognized by T-cells 3 (SART3); paired box protein Pax-5 (PAX5); proacrosin binding protein sp32 (OY-TES I); lymphocyte-specific protein tyrosine kinase (LCK); A kinase anchor protein 4 (AKAP-4); synovial sarcoma, X breakpoint 2 (SSX2); receptor for advanced glycation endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; leukocyte-associated immunoglobulin-like receptor 1 (LAIRI); Fc fragment of IgA receptor (FCAR or CD89); leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fe receptor-like 5 (FCRL5); and immunoglobulin lambda-like polypeptide 1 (IGLL1). In some embodiments, the target is an epitope of the tumor associated antigen presented in an MHC.

In some embodiments, the tumor antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

In some embodiments, the antigen binding domain binds to an epitope of a target or tumor associated antigen (TAA) presented in a major histocompatibility complex (MHC) molecule. In some embodiments, the TAA is a cancer testis antigen. In some embodiments, the cancer testis antigen is selected from the group consisting of acrosin binding protein (ACRBP; CT23, OY-TES-1, SP32; NCBI Gene ID: 84519), alpha fetoprotein (AFP; AFPD, FETA, HPAFP; NCBI Gene ID: 174); A-kinase anchoring protein 4 (AKAP4; AKAP 82, AKAP-4, AKAP82, CT99, FSC1, HI, PRKA4, hAKAP82, p82; NCBI Gene ID: 8852), ATPase family AAA domain containing 2 (ATAD2; ANCCA, CT137, PRO2000; NCBI Gene ID: 29028), kinetochore scaffold 1 (KNL1; AF15Q14, CASC5, CT29, D40, MCPH4, PPP1R55, Spc7, hKNL-1, hSpc105; NCBI Gene ID: 57082), centrosomal protein 55 (CEP55; C10orf3, CT111, MARCH, URCC6; NCBI Gene ID: 55165), cancer/testis antigen IA (CTAG1A; ESO1; CT6.1; LAGE-2; LAGE2A; NY-ESO-1; NCBI Gene ID: 246100), cancer/testis antigen 1B (CTAG1B; CT6.1, CTAG, CTAG1, ESO1, LAGE-2, LAGE2B, NY-ESO-1; NCBI Gene ID: 1485), cancer/testis antigen 2 (CTAG2; CAMEL, CT2, CT6.2, CT6.2a, CT6.2b, ESO2, LAGE-1, LAGE2B; NCBI Gene ID: 30848), CCCTC-binding factor like (CTCFL; BORIS, CT27, CTCF-T, HMGB1L1, dJ579F20.2; NCBI Gene ID: 140690), catenin alpha 2 (CTNNA2; CAP-R, CAPR, CDCBM9, CT114, CTNR; NCBI Gene ID: 1496), cancer/testis antigen 83 (CT83; CXorf6l, KK-LC-1, KKLC1; NCBI Gene ID: 203413), cyclin A1 (CCNA1; CT146; NCBI Gene ID: 8900), DEAD-box helicase 43 (DDX43; CT13, HAGE; NCBI Gene ID: 55510), developmental pluripotency associated 2 (DPPA2; CT100, ECAT15-2, PESCRG1; NCBI Gene ID: 151871), fetal and adult testis expressed 1 (FATE1; CT43, FATE; NCBI Gene ID: 89885), FMR1 neighbor (FMR1NB; CT37, NY-SAR-35, NYSAR35; NCBI Gene ID: 158521), HORMA domain containing 1 (HORMAD1; CT46, NOHMA; NCBI Gene ID: 84072), insulin like growth factor 2 mRNA binding protein 3 (IGF2BP3; CT98, IMP-3, IMP3, KOC, KOC1, VICKZ3; NCBI Gene ID: 10643), leucine zipper protein 4 (LUZP4; CT-28, CT-8, CT28, HOM-TES-85; NCBI Gene ID: 51213), lymphocyte antigen 6 family member K (LY6K; CT97, HSJ001348, URLC10, ly-6K; NCBI Gene ID: 54742), maelstrom spermatogenic transposon silencer (MAEL; CT128, SPATA35; NCBI Gene ID: 84944), MAGE family member A1 (MAGEA1; CT1.1, MAGE1; NCBI Gene ID: 4100); MAGE family member A3 (MAGEA3; CT1.3, HIP8, HYPD, MAGE3, MAGEA6; NCBI Gene ID: 4102); MAGE family member A4 (MAGEA4; CT1.4, MAGE-41, MAGE-X2, MAGE4, MAGE4A, MAGE4B; NCBI Gene ID: 4103); MAGE family member A11 (MAGEA11; CT1.11, MAGE-11, MAGE11, MAGEA-11; NCBI Gene ID: 4110); MAGE family member C1 (MAGEC1; CT7, CT7.1; NCBI Gene ID: 9947); MAGE family member C₂ (MAGEC2; CT10, HCA587, MAGEE1; NCBI Gene ID: 51438); MAGE family member D1 (MAGED1; DLXIN-1, NRAGE; NCBI Gene ID: 9500); MAGE family member D2 (MAGED2; 11B6, BARTS5, BCG-1, BCG1, HCA10, MAGE-D2; NCBI Gene ID: 10916), kinesin family member 20B (KIF20B; CT90, KRMP1, MPHOSPH1, MPP-1, MPP1; NCBI Gene ID: 9585), NUF2 component of NDC80 kinetochore complex (NUF2; CDCA1, CT106, NUF2R; NCBI Gene ID: 83540), nuclear RNA export factor 2 (NXF2; CT39, TAPL-2, TCP11X2; NCBI Gene ID: 56001), PAS domain containing repressor 1 (PASD1; CT63, CT64, OXTES1; NCBI Gene ID: 139135), PDZ binding kinase (PBK; CT84, HEL164, Nori-3, SPK, TOPK; NCBI Gene ID: 55872), piwi like RNA-mediated gene silencing 2 (PIWIL2; CT80, HILI, PIWIL1L, mili; NCBI Gene ID: 55124), preferentially expressed antigen in melanoma (PRAME; CT130, MAPE, OIP-4, OIP4; NCBI Gene ID: 23532), sperm associated antigen 9 (SPAG9; CT89, HLC-6, HLC4, HLC6, JIP-4, JIP4, JLP, PHET, PIG6; NCBI Gene ID: 9043), sperm protein associated with the nucleus, X-linked, family member A1 (SPANXA1; CT11.1, CT11.3, NAP-X, SPAN-X, SPAN-Xa, SPAN-Xb, SPANX, SPANX-A; NCBI Gene ID: 30014), SPANX family member A2 (SPANXA2; CT11.1, CT11.3, SPANX, SPANX-A, SPANX-C, SPANXA, SPANXC; NCBI Gene ID: 728712), SPANX family member C (SPANXC; CT11.3, CTp11, SPANX-C, SPANX-E, SPANXE; NCBI Gene ID: 64663), SPANX family member D (SPANXD; CT11.3, CT11.4, SPANX-C, SPANX-D, SPANX-E, SPANXC, SPANXE, dJ171K16.1; NCBI Gene ID: 64648), SSX family member 1 (SSX1; CT5.1, SSRC; NCBI Gene ID: 6756), SSX family member 2 (SSX2; CT5.2, CT5.2A, HD21, HOM-MEL-40, SSX; NCBI Gene ID: 6757), synaptonemal complex protein 3 (SYCP3; COR1, RPRGL4, SCP3, SPGF4; NCBI Gene ID: 50511), testis expressed 14, intercellular bridge forming factor (TEX14; CT113, SPGF23; NCBI Gene ID: 56155), transcription factor Dp family member 3 (TFDP3; CT30, DP4, HCA661; NCBI Gene ID: 51270), serine protease 50 (PRSS50; CT20, TSP50; NCBI Gene ID: 29122), TTK protein kinase (TTK; CT96, ESK, MPH1, MPS1, MPS1L1, PYT; NCBI Gene ID: 7272) and zinc finger protein 165 (ZNF165; CT53, LD65, ZSCAN7; NCBI Gene ID: 7718). T cell receptors (TCRs) and TCR-like antibodies that bind to an epitope of a cancer testis antigen presented in a major histocompatibility complex (MHC) molecule are known in the art and can be used in the herein described heterodimers. Cancer testis antigens associated with neoplasia are summarized, e.g., in Gibbs, et al., Trends Cancer 2018 October; 4(10):701-712 and the CT database website at cta.lncc.br/index.php. Illustrative TCRs and TCR-like antibodies that bind to an epitope of NY-ESO-1 presented in an MHC are described, e.g., in Stewart-Jones, et al., Proc Natl Acad Sci USA. 2009 Apr. 7; 106(14):5784-8; WO2005113595, WO2006031221, WO2010106431, WO2016177339, WO2016210365, WO2017044661, WO2017076308, WO2017109496, WO2018132739, WO2019084538, WO2019162043, WO2020086158 and WO2020086647. Illustrative TCRs and TCR-like antibodies that bind to an epitope of PRAME presented in an MHC are described, e.g., in WO2011062634, WO2016142783, WO2016191246, WO2018172533, WO2018234319 and WO2019109821. Illustrative TCRs and TCR-like antibodies that bind to an epitope of a MAGE variant presented in an MHC are described, e.g., in WO2007032255, WO2012054825, WO2013039889, WO2013041865, WO2014118236, WO2016055785, WO2017174822, WO2017174823, WO2017174824, WO2017175006, WO2018097951, WO2018170338, WO2018225732 and WO2019204683. Illustrative TCRs and TCR-like antibodies that bind to an epitope of alpha fetoprotein (AFP) presented in an MHC are described, e.g., in WO2015011450. Illustrative TCRs and TCR-like antibodies that bind to an epitope of SSX2 presented in an MHC are described, e.g., in WO2020063488. Illustrative TCRs and TCR-like antibodies that bind to an epitope of KK-LC-1 (CT83) presented in an MHC are described, e.g., in WO2017189254.

Examples of cell therapies include: Algenpantucel-L, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, CryoStim, AlloStim, lentiviral transduced huCART-meso cells, CART-22 cells, EGFRt/19-28z/4-1BBL CAR T cells, autologous 4H111-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, and CSG-005.

In some embodiments the one or more additional co-administered therapeutic agents can be categorized by their mechanism of action, e.g., into the following groups:
agents targeting adenosine deaminase, such as pentostatin or cladribine;
agents targeting ATM, such as AZD1390;
agents targeting MET, such as savolitinib, capmatinib, tepotinib, ABT-700, AG213, JNJ-38877618 (OMO-1), merestinib, HQP-8361, BMS-817378, or TAS-115;
agents targeting mitogen-activated protein kinase, such as antroquinonol, binimetinib, cobimetinib, selumetinib, trametinib, uprosertib, mirdametinib (PD-0325901), pimasertib, refametinib, or compounds disclosed in WO2011008709, WO2013112741, WO2006124944, WO2006124692, WO2014064215, WO2018005435, Zhou, et al., Cancer Lett. 2017 Nov. 1, 408:130-137, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42, or Hu, et al., Bioorg Med Chem Lett. (2011) 21(16):4758-61;
agents targeting thymidine kinase, such as aglatimagene besadenovec (ProstAtak, PancAtak, GliAtak, GMCI, or AdV-tk);
agents targeting targeting an interleukin pathway, such as pegilodecakin (AM-0010) (pegylated IL10), CA-4948 (IRAK4 inhibitor);
agents targeting cytochrome P450 family members, such as letrozole, anastrozole, aminoglutethimide, megestrol acetate (MEGACE®), exemestane, formestane, fadrozole, vorozole (RIVISOR®), letrozole (FEMARA®), or anastrozole (ARIMIDEX®);
agents targeting CD73, such as a CD73 inhibitor (e.g., quemliclustat (AB680)) or an anti-CD73 antibody (e.g., oleclumab);
agents targeting DKK3, such as MTG-201;
agents targeting EEF1A2, such as plitidepsin;
agents targeting EIF4A1, such as rohinitib;
agents targeting endoglin, such as TRC105 (carotuximab);
agents targeting exportin-1, such as eltanexor;
agents targeting fatty acid amide hydrolase, such as compounds disclosed in WO2017160861;
agents targeting heat shock protein 90 beta family member 1, such as anlotinib;
agents targeting lactotransferrin, such as ruxotemitide (LTX-315);
agents targeting lysyl oxidase, such as compounds disclosed in U.S. Pat. Nos. 4,965,288, 4,997,854, 4,943,593, 5,021,456, 5,059,714, 5,120,764, 5,182,297, 5,252,608, or US20040248871;
agents targeting MAGE family members, such as KITE-718, MAGE-A10C796T, or MAGE-A10 TCR;
agents targeting MDM2, such as ALRN-6924, CMG-097, milademetan monotosylate monohydrate (DS-3032b), or AMG-232;
agents targeting MDM4, such as ALRN-6924;
agents targeting melan-A, such as MART-1 F5 TCR engineered PBMCs;
agents targeting mesothelin, such as CSG-MESO or TC-210;
agents targeting METAP2, such as M8891 or APL-1202;
agents targeting NLRP3, such as BMS-986299;
agents targeting oxoglutarate dehydrogenase, such as devimistat (CPI-613);
agents targeting placenta growth factor, such as aflibercept;
agents targeting SLC10A3, such as compounds disclosed in WO2015148954, WO2012082647, or WO2017160861;
agents targeting transforming growth factor alpha (TGFa), such as compounds disclosed in WO2019103203;
agents targeting tumor protein p53, such as kevetrin (stimulator);
agents targeting vascular endothelial growth factor A, such as aflibercept;
agents targeting vascular endothelial growth factor receptor, such as fruquintinib or MP0250;
agents targeting VISTA, such as CA-170, or HMBD-002;
agents targeting WEE1, such as adavosertib (AZD-1775);
small molecule inhibitors targeting ABL1, such as imatinib, rebastinib, asciminib, or ponatinib (ICLUSIG®);
small molecule antagonists targeting adenosine receptor, such as CPI-444, AZD-4635, preladenant, etrumadenant (AB928), or PBF-509;
small molecule inhibitors targeting arachidonate 5-lipoxygenase, such as meclofenamate sodium or zileuton;
small molecule inhibitors targeting ATR serine/threonine kinase, such as BAY-937, ceralasertib (AZD6738), AZD6783, VX-803, or VX-970 (berzosertib);
small molecule inhibitors targeting AXL receptor tyrosine kinase, such as bemcentinib (BGB-324), SLC-0211, or gilteritinib (Axl/Flt3);
small molecule inhibitors targeting Bruton's tyrosine kinase (BTK), such as (S)-6-amino-9-(1-(but-2-ynoyl)pyrrolidin-3-yl)-7-(4-phenoxyphenyl)-7H-purin-8(9H)-one, acalabrutinib (ACP-196), zanubrutinib (BGB-3111), CB988, poseltinib (HM71224), ibrutinib (Imbruvica), M-2951 (evobrutinib), tirabrutinib (ONO-4059), rilzabrutinib (PRN-1008), spebrutinib (CC-292), vecabrutinib, ARQ-531 (MK-1026), SHR-1459, DTRMWXHS-12, or TAS-5315;
small molecule inhibitors targeting neurotrophic receptor tyrosine kinase such as larotrectinib, entrectinib, or selitrectinib (LOXO-195);
small molecule inhibitors targeting ROS proto-oncogene 1, receptor tyrosine kinase, such as entrectinib, repotrectinib (TPX-0005), or lorlatinib;
small molecule inhibitors targeting SRC proto-oncogene, non-receptor tyrosine kinase, such as VAL-201, tirbanibulin (KX2-391), or ilginatinib maleate (NS-018);
small molecule inhibitors targeting B-cell lymphoma 2, such as navitoclax (ABT-263), venetoclax (ABT-199, RG-7601), or AT-101 (gossypol);
small molecule inhibitors targeting bromodomain and external domain (BET) bromodomain containing protein, such as ABBV-744, INCB-054329, INCB057643, AZD-5153, ABT-767, BMS-986158, CC-90010, NHWD-870, ODM-207, ZBC246, ZEN3694, CC-95775 (FT-1101), mivebresib, BI-894999, PLX-2853, PLX-51107, CPI-0610, or GS-5829;
small molecule inhibitors targeting carbohydrate sulfotransferase 15, such as STNM-01;
small molecule inhibitors targeting carbonic anhydrase, such as polmacoxib, acetazolamide, or methazolamide;
small molecule inhibitors targeting catenin beta 1, such as CWP-291, or PRI-724;
small molecule antagonists targeting a C—C motif chemokine receptor, such as CCX-872, BMS-813160 (CCR2/CCR5) or MK-7690 (vicriviroc);
small molecule antagonists targeting a C—X—C motif chemokine receptor (e.g., CXCR4), blixafortide;
small molecule inhibitors targeting cereblon, such as avadomide (CC-122), CC-92480, CC-90009, or iberdomide;
small molecule inhibitors targeting checkpoint kinase 1, such as SRA737;
small molecule inhibitors targeting a complement component, such as Imprime PGG (Biothera Pharmaceuticals);
small molecule inhibitor targeting a C—X—C motif chemokine ligand (e.g., CXCL12), such as olaptesed pegol (NOX-A12);
small molecule inhibitors targeting cytochrome P450 family, such as ODM-209, LAE-201, seviteronel (VT-464), CFG920, abiraterone, or abiraterone acetate;
small molecule inhibitors targeting DEAD-box helicase 5, such as supinoxin (RX-5902);
small molecule inhibitors targeting DGKa, e.g., such as described in WO2021130638;
small molecule inhibitors targeting diablo IAP-binding mitochondrial protein, such as BI-891065;
small molecule inhibitors targeting dihydrofolate reductase, such as pralatrexate or pemetrexed disodium;
small molecule inhibitors targeting DNA dependent protein kinase, such as MSC2490484A (nedisertib), VX-984, AsiDNA (DT-01), LXS-196, or sotrastaurin;
small molecule inhibitors targeting MARCKS, such as BIO-11006;
small molecule inhibitors targeting RIPK1, such as GSK-3145094;
small molecule inhibitors targeting Rho associated coiled-coil containing protein kinase, such as AT13148 or KD025;
small molecule inhibitors targeting DNA topoisomerase, such as irinotecan, firtecan pegol, or amrubicin;
small molecule inhibitors targeting dopamine receptor D2, such as ONC-201;
small molecule inhibitors targeting DOT1 like histone lysine methyltransferase, such as pinometostat (EPZ-5676);
small molecule inhibitors targeting EZH2, such as tazemetostat, CPI-1205, or PF-06821497;
small molecule inhibitors targeting fatty acid synthase, such as TVB-2640 (Sagimet Biosciences);
small molecule inhibitors targeting fibroblast growth factor receptor 2 (FGFR2), such as bemarituzumab (FPA144);
small molecule inhibitors targeting focal adhesion kinase (FAK, PTK2), such as VS-4718, defactinib, or GSK2256098;
small molecule inhibitors targeting folate receptor 1, such as pralatrexate;
small molecule inhibitors targeting FOXM1, such as thiostrepton;
small molecule inhibitors targeting galectin 3, such as belapectin (GR-MD-02);
small molecule antagonists targeting glucocorticoid receptor, such as relacorilant (CORT-125134);
small molecule inhibitors targeting glutaminase include without limitation CB-839 (telaglenastat), or bis-2-(5-phenylacetamido-1,3,4-thiadiazol-2-yl)ethyl sulfide (BPTES);
small molecule inhibitors targeting GNRHR, such as elagolix, relugolix, or degarelix;
small molecule inhibitors targeting EPAS1, such as belzutifan (PT-2977 (Merck & Co.));
small molecule inhibitors targeting isocitrate dehydrogenase (NADP(+)), such as limitation ivosidenib (AG-120), vorasidenib (AG-881) (IDH1 and IDH2), IDH-305, or enasidenib (AG-221);
small molecule inhibitors targeting lysine demethylase 1A, such as CC-90011;
small molecule inhibitors targeting MAPK interacting serine/threonine kinase, such as tomivosertib (eFT-508);
small molecule inhibitors targeting notch receptor, such as AL-101 (BMS-906024);
small molecule inhibitors targeting polo like kinase 1 (PLK1), such as volasertib or onvansertib;
small molecule inhibitors targeting poly(ADP-ribose) polymerase (PARP), such as olaparib (MK7339), rucaparib, veliparib, talazoparib, ABT-767, pamiparib (BGB-290), fluazolepali (SHR-3162), niraparib (JNJ-64091742), stenoparib (2X-121 (e-7499)), simmiparib, IMP-4297, SC-10914, IDX-1197, HWH-340, CEP 9722, CEP-8983, E7016, 3-aminobenzamide, or CK-102;
small molecule inhibitors targeting polycomb protein EED, such as MAK683;
small molecule inhibitors targeting porcupine O-acyltransferase, such as WNT-974;
small molecule inhibitors targeting prostaglandin-endoperoxide synthase, such as HP-5000, lornoxicam, ketorolac tromethamine, bromfenac sodium, otenaproxesul (ATB-346), mofezolac, GLY-230, TRK-700, diclofenac, meloxicam, parecoxib, etoricoxib, celecoxib, AXS-06, diclofenac potassium, reformulated celecoxib (DRGT-46), AAT-076, meisuoshuli, lumiracoxib, meloxicam, valdecoxib, zaltoprofen, nimesulide, anitrazafen, apricoxib, cimicoxib, deracoxib, flumizole, firocoxib, mavacoxib, pamicogrel, parecoxib, robenacoxib, rofecoxib, rutecarpine, tilmacoxib, zaltoprofen, or imrecoxib;
small molecule inhibitors targeting protein arginine N methyltransferase, such as MS203, PF-06939999, GSK3368715, or GSK3326595;
small molecule inhibitors targeting PTPN11, such as TNO155 (SHP-099), RMC-4550, JAB-3068, RMC-4630 (SAR442720), or compounds disclosed in WO2018172984 or WO2017211303;
small molecule antagonist targeting retinoic acid receptor, such as tamibarotene (SY-1425);
small molecule inhibitors targeting ribosomal protein S6 kinase B1, such as MSC2363318A;
small molecule inhibitors targeting S100 calcium binding protein A9, such as tasquinimod;
small molecule inhibitors targeting selectin E, such as uproleselan sodium (GMI-1271);
small molecule inhibitors targeting SF3B1, such as H3B-8800;
small molecule inhibitors targeting Sirtuin-3, such as YC8-02;
small molecule inhibitors targeting SMO, such as sonidegib (Odomzo®, formerly LDE-225), vismodegib (GDC-0449), glasdegib (PF-04449913), itraconazole, or patidegib, taladegib;
small molecule antagonists targeting somatostatin receptor, such as OPS-201;
small molecule inhibitors targeting sphingosine kinase 2, such as opaganib (Yeliva®, ABC294640);
small molecule inhibitors targeting STAT3, such as napabucasin (BBI-608);
small molecule inhibitors targeting tankyrase, such as G007-LK or stenoparib (2X-121 (e-7499));
small molecule inhibitors targeting TFGBR1, such as galunisertib, PF-06952229;
small molecule inhibitors targeting thymidylate synthase, such as idetrexed (ONX-0801);
small molecule inhibitors targeting tumor protein p53, such as CMG-097;
small molecule inhibitors targeting valosin-containing protein, such as CB-5083;
small molecule inhibitors targeting WT1, such as ombipepimut-S(DSP-7888);
small molecule agonists targeting adenosine receptor, such as namodenoson (CF102);
small molecule agonist(s) targeting asparaginase, such as crisantaspase (Erwinase®), GRASPA (ERY-001, ERY-ASP), calaspargase pegol, or pegaspargase;
small molecule agonists targeting CCAAT enhancer binding protein alpha, such as MTL-501;
small molecule agonists targeting cytochrome P450 family, such as mitotane;
small molecule agonists targeting DExD/H-box helicase 58, such as RGT-100;
small molecule agonists targeting GNRHR, such as leuprorelin acetate, leuprorelin acetate sustained release depot (ATRIGEL), triptorelin pamoate, or goserelin acetate;
small molecule agonists targeting GRB2, such as prexigebersen (BP1001);
small molecule agonists targeting NFE2L2, such as omaveloxolone (RTA-408);
small molecule agonists targeting NOD2, such as mifamurtide (liposomal);
small molecule agonists targeting RAR-related orphan receptor gamma, such as cintirorgon (LYC-55716);
small molecule agonists targeting retinoic acid receptor (RAR), such as tretinoin;
small molecule agonists targeting STING1, such as ADU-S100 (MIW-815), SB-11285, MK-1454, SR-8291, AdVCA0848, GSK-532, SYN-STING, MSA-1, SR-8291, cyclic-GAMP (cGAMP), or cyclic-di-AMP;
small molecule agonists targeting thyroid hormone receptor beta, such as levothyroxine sodium;
small molecule agonists targeting tumor necrosis factor, such as tasonermin;
antisense agents targeting baculoviral IAP repeat containing 5, such as EZN-3042;
antisense agents targeting GRB2, such as prexigebersen;
antisense agents targeting heat shock protein 27, such as apatorsen;
antisense agents targeting STAT3, such as danvatirsen (IONIS-STAT3-2.5Rx);
gene therapies targeting a C—C motif chemokine receptor, such as SB-728-T;
gene therapies targeting an interleukin, such as EGENE-001, tavokinogene telseplasmid, nogapendekin alfa (ALT-803), NKTR-255, NIZ-985 (hetIL-15), SAR441000, or MDNA-55;
antibodies targeting claudin 18, such as claudiximab;
antibodies targeting clusterin, such as AB-16B5;
antibodies targeting a complement component, such as ravulizumab (ALXN-1210);
antibodies targeting a C—X—C motif chemokine ligand, such as BMS-986253 (HuMax-Inflam);
antibodies targeting delta like canonical Notch ligand 4 (DLL4), such as demcizumab, navicixizumab (DLL4/VEGF);
antibodies targeting EPH receptor A3, such as fibatuzumab (KB-004);
antibodies targeting epithelial cell adhesion molecule, such as oportuzumab monatox (VB4-845);
antibodies targeting fibroblast growth factor, such as GAL-F2, B-701 (vofatamab);
antibodies targeting hepatocyte growth factor, such as MP-0250;
antibodies targeting an interleukin, such as canakinumab (ACZ885), gevokizumab (VPM087), CJM-112, guselkumab, talacotuzumab (JNJ-56022473), siltuximab, or tocilizumab;
antibodies targeting LRRC15, such as ABBV-085 or cusatuzumab (ARGX-110);
antibodies targeting mesothelin, such as BMS-986148, SEL-403, or anti-MSLN-MMAE;
antibodies targeting myostatin, such as landogrozumab;
antibodies targeting notch receptor, such as tarextumab;
antibodies targeting TGFB1 (TGFb1), such as SAR439459, ABBV-151, NIS793, SRK-181, XOMA089, or compounds disclosed in WO2019103203;
vaccines targeting fms related receptor tyrosine kinase, such as HLA-A2402/HLA-A0201 restricted epitope peptide vaccine;
vaccines targeting heat shock protein 27, such as PSV-AML (PhosphoSynVax);
vaccines targeting PD-L1, such as IO-120+IO-103 (PD-L1/PD-L2 vaccines) or IO-103;
vaccines targeting tumor protein p53, such as MVA-p53;
vaccines targeting WT1, such as WT-1 analog peptide vaccine (WT1-CTL);
cell therapies targeting baculoviral IAP repeat containing 5, such as tumor lysate/MUC1/survivin PepTivator-loaded dendritic cell vaccine;
cell therapies targeting carbonic anhydrase, such as DC-Ad-GMCAIX;
cell therapies targeting C—C motif chemokine receptor, such as CCR5-SBC-728-HSPC;
cell therapies targeting folate hydrolase 1, such as CIK-CAR.PSMA or CART-PSMA-TGFβRDN;
cell therapies targeting GSTP1, such as CPG3-CAR (GLYCAR);
cell therapies targeting HLA-A, such as FH-MCVA2TCR or NeoTCR-P1;
cell therapies targeting an interleukin, such as CST-101;
cell therapies targeting KRAS, such as anti-KRAS G12D mTCR PBL;
cell therapies targeting MET, such as anti-cMet RNA CAR T;
cell therapies targeting MUC16, such as JCAR-020;
cell therapies targeting PD-1, such as PD-1 knockout T cell therapy (esophageal cancer/NSCLC);
cell therapies targeting PRAME, such as BPX-701;
cell therapies targeting transforming protein E7, such as KITE-439;
cell therapies targeting WTi, such as WTI-CTL, ASP-7517, or JTCR-016.

### Exemplified Combination Therapies

### Lymphoma or Leukemia Combination Therapy

Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541, bortezomib (VELCADE®), bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, doxorubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, doxorubicin, doxorubicin hydrochloride, DT-PACE (dexamethasone, thalidomide, cisplatin, doxorubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, doxorubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WNIG, Omrix), oxaliplatin, paclitaxel, palbociclib (PD0332991), pegfilgrastim, PEGylated liposomal doxorubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R-CHOP (rituximab and CHOP), R-CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CC1-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf®), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP (cyclophosphamide, doxorubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including rituximab (R) and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP (rituximab, cyclophosphamide, doxorubicin, vincristine, and prednisone), R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CCI-779), everolimus (RAD001), BMS-345541, curcumin, SAHA, thalidomide, lenalidomide (REVLIMID®, CC-5013), and geldanamycin (17 AAG).

### Waldenstrom's Macroglobulinemia Combination Therapy

Therapeutic agents used to treat Waldenstrom's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, doxorubicin hydrochloride, DT-PACE, enzastaurin, epoetin alfa, epratuzumab (hLL2- anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as tisagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal doxorubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WT1 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-cell Lymphoma (DLBCL) Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and RICE. In some embodiments therapeutic agents used to treat DLBCL include rituximab (Rituxan®), cyclophosphamide, doxorubicin hydrochloride (hydroxydaunorubicin), vincristine sulfate (Oncovin®), prednisone, bendamustine, ifosfamide, carboplatin, etoposide, ibrutinib, polatuzumab vedotin piiq, bendamustine, copanlisib, lenalidomide (Revlimid®), dexamethasone, cytarabine, cisplatin, Yescarta®, Kymriah®, Polivy®(polatuzumab vedotin), BR (bendamustine (Treanda®), gemcitabine, oxiplatin, oxaliplatin, tafasitamab, polatuzumab, cyclophosphamide, or combinations thereof. In some embodiments therapeutic agents used to treat DLBCL include R-CHOP (rituximab+cyclophosphamide+doxorubicin hydrochloride (hydroxydaunorubicin)+vincristine sulfate (Oncovin®), +prednisone), rituximab+bendamustine, R-ICE (Rituximab+Ifosfamide+Carboplatin+Etoposide), rituximab+lenalomide, R-DHAP (rituximab+dexamethasone+high-dose cytarabine (Ara C)+cisplatin), Polivy®(polatuzumab vedotin)+BR (bendamustine (Treanda®) and rituximab (Rituxan®), R-GemOx (Gemcitabine+oxaliplatin+rituximab), Tafa-Len (tafasitamab+lenalidomide), Tafasitamab+Revlimid®, polatuzumab+bendamustine, Gemcitabine+oxaliplatin, R-EPOCH (rituximab+etoposide phosphate+prednisone+vincristine sulfate (Oncovin®)+cyclophosphamide+doxorubicin hydrochloride (hydroxydaunorubicin)), or CHOP (cyclophosphamide+doxorubicin hydrochloride (hydroxydaunorubicin)+vincristine sulfate (Oncovin®)+prednisone). In some embodiments therapeutic agents used to treat DLBCL include tafasitamab, glofitamab, epcoritamab, Lonca-T (loncastuximab tesirine), Debio-1562, polatuzumab, Yescarta, JCAR017, ADCT-402, brentuximab vedotin, MT-3724, odronextamab, Auto-03, Allo-501A, or TAK-007.

### Chronic Lymphocytic Leukemia Combination Therapy

Therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### High Risk Myelodysplastic Syndrome (HR MDS) Combination Therapy

Therapeutic agents used to treat HR MDS include azacitidine (Vidaza®), decitabine (Dacogen®), lenalidomide (Revlimid®), cytarabine, idarubicin, daunorubicin, and combinations thereof. In some embodiments combinations include cytarabine+daunorubicin and cytarabine+idarubicin. In some embodiments therapeutic agents used to treat HR MDS include pevonedistat, venetoclax, sabatolimab, guadecitabine, rigosertib, ivosidenib, enasidenib, selinexor, BGB324, DSP-7888, or SNS-301.

### Low Risk Myelodysplastic Syndrome (LR MDS) Combination Therapy

Therapeutic agents used to treat LR MDS include lenalidomide, azacytidine, and combinations thereof. In some embodiments therapeutic agents used to treat LR MDS include roxadustat, luspatercept, imetelstat, LB-100, or rigosertib.

### High Risk Myelodysplastic Syndrome (HR MDS) Combination Therapy

Therapeutic agents used to treat HR MDS include azacitidine (Vidaza®), decitabine (Dacogen®), lenalidomide (Revlimid®), cytarabine, idarubicin, daunorubicin, and combinations thereof. In some embodiments combinations include cytarabine+daunorubicin and cytarabine+idarubicin. In some embodiments therapeutic agents used to treat HR MDS include pevonedistat, venetoclax, sabatolimab, guadecitabine, rigosertib, ivosidenib, enasidenib, selinexor, BGB324, DSP-7888, or SNS-301.

### Low Risk Myelodysplastic Syndrome (LR MDS) Combination Therapy

Therapeutic agents used to treat LR MDS include lenalidomide, azacytidine, and combinations thereof. In some embodiments therapeutic agents used to treat LR MDS include roxadustat, luspatercept, imetelstat, LB-100, or rigosertib.

### Acute Myeloid Leukemia (AML) Combination Therapy

Therapeutic agents used to treat AML include cytarabine, idarubicin, daunorubicin, midostaurin (Rydapt®), venetoclax, azacitidine, ivasidenib, gilteritinib, enasidenib, low-dose cytarabine (LoDAC), mitoxantrone, fludarabine, granulocyte-colony stimulating factor, idarubicin, gilteritinib (Xospata®), enasidenib (Idhifa®), ivosidenib (Tibsovo®), decitabine (Dacogen®), mitoxantrone, etoposide, Gemtuzumab ozogamicin (Mylotarg®), glasdegib (Daurismo®), and combinations thereof. In some embodiments therapeutic agents used to treat AML include FLAG-Ida (fludarabine, cytarabine (Ara-C), granulocyte-colony stimulating factor (G-CSF) and idarubicin), cytarabine+idarubicin, cytarabine+daunorubicin+midostaurin, venetoclax+azacitidine, cytarabine+daunorubicin, or MEC (mitoxantrone, etoposide, and cytarabine). In some embodiments, therapeutic agents used to treat AML include pevonedistat, venetoclax, sabatolimab, eprenetapopt, or lemzoparlimab.

### Multiple Myeloma (MM) Combination Therapy

Therapeutic agents used to treat MM include lenalidomide, bortezomib, dexamethasone, daratumumab (Darzalex®), pomalidomide, Cyclophosphamide, Carfilzomib (Kyprolis®), Elotuzumab (Empliciti), and combinations thereof. In some embodiments therapeutic agents used to treat MM include RVS (lenalidomide+bortezomib+dexamethasone), RevDex (lenalidomide plus dexamethasone), CYBORD (Cyclophosphamide+Bortezomib+Dexamethasone), Vel/Dex (bortezomib plus dexamethasone), or PomDex (Pomalidomide+low-dose dexamethasone). In some embodiments therapeutic agents used to treat MM include JCARH125, TAK-573, belantamab-m, ide-cel (CAR-T).

### Breast Cancer Combination Therapy

Therapeutic agents used to treat breast cancer include albumin-bound paclitaxel, anastrozole, atezolizumab, capecitabine, carboplatin, cisplatin, cyclophosphamide, docetaxel, doxorubicin, epirubicin, everolimus, exemestane, fluorouracil, fulvestrant, gemcitabine, Ixabepilone, lapatinib, letrozole, methotrexate, mitoxantrone, paclitaxel, pegylated liposomal doxorubicin, pertuzumab, tamoxifen, toremifene, trastuzumab, vinorelbine, and any combinations thereof. In some embodiments therapeutic agents used to treat breast cancer (e.g., HR+/−/HER2+/−) include trastuzumab (Herceptin®), pertuzumab (Perjeta®), docetaxel, carboplatin, palbociclib (Ibrance®), letrozole, trastuzumab emtansine (Kadcyla®), fulvestrant (Faslodex®), olaparib (Lynparza®), eribulin, tucatinib, capecitabine, lapatinib, everolimus (Afinitor®), exemestane, eribulin mesylate (Halaven®), and combinations thereof. In some embodiments therapeutic agents used to treat breast cancer include trastuzumab+pertuzumab+docetaxel, trastuzumab+pertuzumab+docetaxel+carboplatin, palbociclib+letrozole, tucatinib+capecitabine, lapatinib+capecitabine, palbociclib+fulvestrant, or everolimus+exemestane. In some embodiments therapeutic agents used to treat breast cancer include trastuzumab deruxtecan (Enhertu®), datopotamab deruxtecan (DS-1062), enfortumab vedotin (Padcev®), balixafortide, elacestrant, or a combination thereof. In some embodiments therapeutic agents used to treat breast cancer include balixafortide+eribulin.

### Triple Negative Breast Cancer (TNBC) Combination Therapy

Therapeutic agents used to treat TNBC include atezolizumab, cyclophosphamide, docetaxel, doxorubicin, epirubicin, fluorouracil, paclitaxel, and combinations thereof. In some embodiments therapeutic agents used to treat TNBC include olaparib (Lynparza®), atezolizumab (Tecentriq®), paclitaxel (Abraxane®), eribulin, bevacizumab (Avastin®), carboplatin, gemcitabine, eribulin mesylate (Halaven®), sacituzumab govitecan (Trodelvy®), pembrolizumab (Keytruda®), cisplatin, doxorubicin, epirubicin, or a combination thereof. In some embodiments therapeutic agents to treat TNBC include atezolizumab+paclitaxel, bevacizumab+paclitaxel, carboplatin+paclitaxel, carboplatin+gemcitabine, or paclitaxel+gemcitabine. In some embodiments therapeutic agents used to treat TNBC include eryaspase, capivasertib, alpelisib, rucaparib+nivolumab, atezolumab+paclitaxel+gemcitabine+capecitabine+carboplatin, ipatasertib+paclitaxel, ladiratuzumab vedotin+pembrolimab, durvalumab+DS-8201a, trilaciclib+gemcitabine+carboplatin. In some embodiments therapeutic agents used to treat TNBC include trastuzumab deruxtecan (Enhertu®), datopotamab deruxtecan (DS-1062), enfortumab vedotin (Padcev®), balixafortide, adagloxad simolenin, nelipepimut-s (NeuVax®), nivolumab (Opdivo®), rucaparib, toripalimab (Tuoyi®), camrelizumab, capivasertib, durvalumab (Imfinzi®), and combinations thereof. In some embodiments therapeutic agents use to treat TNBC include nivolumab+rucaparib, bevacizumab (Avastin®)+chemotherapy, toripalimab+paclitaxel, toripalimab+albumin-bound paclitaxel, camrelizumab+chemotherapy, pembrolizumab+chemotherapy, balixafortide+eribulin, durvalumab+trastuzumab deruxtecan, durvalumab+paclitaxel, or capivasertib+paclitaxel.

### Bladder Cancer Combination Therapy

Therapeutic agents used to treat bladder cancer include datopotamab deruxtecan (DS-1062), trastuzumab deruxtecan (Enhertu®), erdafitinib, eganelisib, lenvatinib, bempegaldesleukin (NKTR-214), or a combination thereof. In some embodiments therapeutic agents used to treat bladder cancer include eganelisib+nivolumab, pembrolizumab (Keytruda®)+enfortumab vedotin (Padcev®), nivolumab+ipilimumab, duravalumab+tremelimumab, lenvatinib+pembrolizumab, enfortumab vedotin (Padcev®)+pembrolizumab, and bempegaldesleukin+nivolumab.

### Colorectal Cancer (CRC) Combination Therapy

Therapeutic agents used to treat CRC include bevacizumab, capecitabine, cetuximab, fluorouracil, irinotecan, leucovorin, oxaliplatin, panitumumab, ziv-aflibercept, and any combinations thereof. In some embodiments therapeutic agents used to treat CRC include bevacizumab (Avastin®), leucovorin, 5-FU, oxaliplatin (FOLFOX), pembrolizumab (Keytruda®), FOLFIRI, regorafenib (Stivarga®), aflibercept (Zaltrap®), cetuximab (Erbitux®), Lonsurf (Orcantas®), XELOX, FOLFOXIRI, or a combination thereof. In some embodiments therapeutic agents used to treat CRC include bevacizumab+leucovorin+5-FU, bevacizumab+leucovorin+5-FU+oxaliplatin (FOLFOX), bevacizumab+FOLFIRI, bevacizumab+FOLFOX, aflibercept+FOLFIRI, cetuximab+FOLFIRI, cetuximab+FOLFOX, bevacizumab+XELOX, and bevacizumab+FOLFOXIRI. In some embodiments therapeutic agents used to treat CRC include binimetinib+encorafenib+cetuximab, trametinib+dabrafenib+panitumumab, trastuzumab+pertuzumab, napabucasin+FOLFIRI+bevacizumab, nivolumab+ipilimumab.

### Esophageal and Esophagogastric Junction Cancer Combination Therapy

Therapeutic agents used to treat esophageal and esophagogastric junction cancer include capecitabine, carboplatin, cisplatin, docetaxel, epirubicin, fluoropyrimidine, fluorouracil, irinotecan, leucovorin, oxaliplatin, paclitaxel, ramucirumab, trastuzumab, and any combinations thereof. In some embodiments therapeutic agents used to treat gastroesophageal junction cancer (GEJ) include herceptin, cisplatin, 5-FU, ramicurimab, or paclitaxel. In some embodiments therapeutic agents used to treat GEJ cancer include ALX-148, AO-176, or IBI-188.

### Gastric Cancer Combination Therapy

Therapeutic agents used to treat gastric cancer include capecitabine, carboplatin, cisplatin, docetaxel, epirubicin, fluoropyrimidine, fluorouracil, Irinotecan, leucovorin, mitomycin, oxaliplatin, paclitaxel, ramucirumab, trastuzumab, and any combinations thereof.

### Head and Neck Cancer Combination Therapy

Therapeutic agents used to treat head & neck cancer include afatinib, bleomycin, capecitabine, carboplatin, cetuximab, cisplatin, docetaxel, fluorouracil, gemcitabine, hydroxyurea, methotrexate, nivolumab, paclitaxel, pembrolizumab, vinorelbine, and any combinations thereof.

Therapeutic agents used to treat head and neck squamous cell carcinoma (HNSCC) include pembrolizumab, carboplatin, 5-FU, docetaxel, cetuximab (Erbitux®), cisplatin, nivolumab (Opdivo®), and combinations thereof. In some embodiments therapeutic agents used to treat HNSCC include pembrolizumab+carboplatin+5-FU, cetuximab+cisplatin+5-FU, cetuximab+carboplatin+5-FU, cisplatin+5-FU, and carboplatin+5-FU. In some embodiments therapeutic agents used to treat HNSCC include durvalumab, durvalumab+tremelimumab, nivolumab+ipilimumab, rovaluecel, pembrolizumab, pembrolizumab+epacadostat, GSK3359609+pembrolizumab, lenvatinib+pembrolizumab, retifanlimab, retifanlimab+enobituzumab, ADU-S100+pembrolizumab, epacadostat+nivolumab+ipilimumab/lirilumab.

### Non-Small Cell Lung Cancer Combination Therapy

Therapeutic agents used to treat non-small cell lung cancer (NSCLC) include afatinib, albumin-bound paclitaxel, alectinib, atezolizumab, bevacizumab, bevacizumab, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, trametinib, trastuzumab, vandetanib, vemurafenib, vinblastine, vinorelbine, and any combinations thereof. In some embodiments therapeutic agents used to treat NSCLC include alectinib (Alecensa®), dabrafenib (Tafinlar®), trametinib (Mekinist®), osimertinib (Tagrisso®), entrectinib (Tarceva®), crizotinib (Xalkori®), pembrolizumab (Keytruda®), carboplatin, pemetrexed (Alimta®), nab-paclitaxel (Abraxane®), ramucirumab (Cyramza®), docetaxel, bevacizumab (Avastin®), brigatinib, gemcitabine, cisplatin, afatinib (Gilotrif®), nivolumab (Opdivo®), gefitinib (Iressa®), and combinations thereof. In some embodiments therapeutic agents used to treat NSCLC include dabrafenib+trametinib, pembrolizumab+carboplatin+pemetrexed, pembrolizumab+carboplatin+paclitaxel, pembrolizumab+carboplatin+nab-paclitaxel, ramucirumab+docetaxel, bevacizumab+carboplatin+pemetrexed, pembrolizumab+pemetrexed+cisplatin, cisplatin+pemetrexed, bevacizumab+carboplatin+nab-paclitaxel, cisplatin+gemcitabine, nivolumab+docetaxel, nivolumab+ipilimumab, carboplatin+pemetrexed, carboplatin+nab-paclitaxel, or pemetrexed+cisplatin+carboplatin. In some embodiments therapeutic agents used to NSCLC include datopotamab deruxtecan (DS-1062), ipilimumab, trastuzumab deruxtecan (Enhertu®), enfortumab vedotin (Padcev®), durvalumab, canakinumab, cemiplimab, nogapendekin alfa, avelumab, tiragolumab, domvanalimab, vibostolimab, ociperlimab, or a combination thereof. In some embodiments therapeutic agents used to treat NSCLC include datopotamab deruxtecan+pembrolizumab, datopotamab deruxtecan+durvalumab, durvalumab+tremelimumab, pembrolizumab+lenvatinib+pemetrexed, pembrolizumab+olaparib, nogapendekin alfa (N-803)+pembrolizumab, tiragolumab+atezolizumab, vibostolimab+pembrolizumab, or ociperlimab+tislelizumab.

### Small Cell Lung Cancer Combination Therapy

Therapeutic agents used to treat small cell lung cancer (SCLC) include atezolizumab, bendamustime, carboplatin, cisplatin, cyclophosphamide, docetaxel, doxorubicin, etoposide, gemcitabine, ipillimumab, irinotecan, nivolumab, paclitaxel, temozolomide, topotecan, vincristine, vinorelbine, and any combinations thereof. In some embodiments therapeutic agents used to treat SCLC include atezolizumab, carboplatin, cisplatin, etoposide, paclitaxel, topotecan, nivolumab, durvalumab, trilaciclib, or combinations thereof. In some embodiments therapeutic agents used to treat SCLC include atezolizumab+carboplatin+etoposide, atezolizumab+carboplatin, atezolizumab+etoposide, or carboplatin+paclitaxel.

### Ovarian Cancer Combination Therapy

Therapeutic agents used to treat ovarian cancer include 5-flourouracil, albumin bound paclitaxel, altretamine, anastrozole, bevacizumab, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, pazopanib, pemetrexed, tamoxifen, topotecan, vinorelbine, and any combinations thereof.

### Pancreatic Cancer Combination Therapies

Therapeutic agents used to treat pancreatic cancer include 5-FU, leucovorin, oxaliplatin, irinotecan, gemcitabine, nab-paclitaxel (Abraxane®), FOLFIRINOX, FOLFOX, XELOX, and combinations thereof. In some embodiments therapeutic agents used to treat pancreatic cancer include 5-FU+leucovorin+oxaliplatin+irinotecan, 5-FU+nanoliposomal irinotecan, cisplatin+gemcitabine, leucovorin+nanoliposomal irinotecan, 5-FU+gemcitabine, and gemcitabine+nab-paclitaxel.

### Endometrial Cancer Combination Therapies

Therapeutic treatments used to treat endometrial cancer include surgery, chemotherapy, radiation therapy, hormone therapy, targeted therapy, and immunotherapy. In some embodiments, the therapeutic treatments include Anti-angiogenesis therapy, Mammalian target of rapamycin (mTOR) inhibitors, Targeted therapy to treat a rare type of uterine cancer.

Therapeutic agents used to treat endometrial cancer include carboplatin, paclitaxel, cisplatin, doxorubicin, ifosfamide, progesterone, anastrozole (Arimidex®), letrozole (Femara®), and exemestane (Aromasin®), pembrolizumab (Keytruda®), lenvatinib (Lenvima®), dostarlimab (Jemperli®), and combinations thereof.

### Prostate Cancer Combination Therapies

Therapeutic agents used to treat prostate cancer include enzalutamide (Xtandi®), leuprolide, trifluridine, tipiracil (Lonsurf), cabazitaxel, prednisone, abiraterone (Zytiga®), docetaxel, mitoxantrone, bicalutamide, LHRH, flutamide, ADT, sabizabulin (Veru-111), and combinations thereof. In some embodiments therapeutic agents used to treat prostate cancer include enzalutamide+leuprolide, trifluridine+tipiracil (Lonsurf), cabazitaxel+prednisone, abiraterone+prednisone, docetaxel+prednisone, mitoxantrone+prednisone, bicalutamide+LHRH, flutamide+LHRH, leuprolide+flutamide, and abiraterone+prednisone+ADT.

### Additional Exemplified Combination Therapies

In some embodiments the compound provided herein is administered with one or more therapeutic agents selected from a PI3K inhibitor, a Trop-2 binding agent, CD47 antagonist, a SIRPα antagonist, a FLT3R agonist, a PD-1 antagonist, a PD-L1 antagonist, an MCL1 inhibitor, a CCR8 binding agent, an HPK1 antagonist, a DGKa inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

In some embodiments the compound provided herein is administered with one or more therapeutic agents selected from a PI3Kd inhibitor (e.g., idealisib), an anti-Trop-2 antibody drug conjugate (e.g., sacituzumab govitecan, datopotamab deruxtecan (DS-1062)), an anti-CD47 antibody or a CD47-blocking agent (e.g., magrolimab, DSP-107, AO-176, ALX-148, letaplimab (IBI-188), lemzoparlimab, TTI-621, TTI-622), an anti-SIRPα antibody (e.g., GS-0189), a FLT3L-Fc fusion protein (e.g., GS-3583), an anti-PD-1 antibody (pembrolizumab, nivolumab, zimberelimab), a small molecule PD-L1 inhibitor (e.g., GS-4224), an anti-PD-L1 antibody (e.g., atezolizumab, avelumab), a small molecule MCL1 inhibitor (e.g., GS-9716), a small molecule HPK1 inhibitor (e.g., GS-6451), a HPK1 degrader (PROTAC; e.g., ARV-766), a small molecule DGKa inhibitor, a small molecule CD73 inhibitor (e.g., quemliclustat (AB680)), an anti-CD73 antibody (e.g., oleclumab), a dual A₂ₐ/A_{2b} adenosine receptor antagonist (e.g., etrumadenant (AB928)), an anti-TIGIT antibody (e.g., tiragolumab, vibostolimab, domvanalimab, AB308), an anti-TREM1 antibody (e.g., PY159), an anti-TREM2 antibody (e.g., PY314), a CD137 agonist (e.g., AGEN-2373), a GITR/OX40 binding agent (e.g., AGEN-1223) and a CAR-T cell therapy (e.g., axicabtagene ciloleucel, brexucabtagene autoleucel, tisagenlecleucel).

In some embodiments the compound provided herein is administered with one or more therapeutic agents selected from idealisib, sacituzumab govitecan, magrolimab, GS-0189, GS-3583, zimberelimab, GS-4224, GS-9716, GS-6451, quemliclustat (AB680), etrumadenant (AB928), domvanalimab, AB308, PY159, PY314, AGEN-1223, AGEN-2373, axicabtagene ciloleucel and brexucabtagene autoleucel.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, ion exchange chromatography, and supercritical fluid chromatography (SFC). Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4th ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product. Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or disatereomerically pure.

Compounds disclosed herein can be prepared from commercially available reagents using the synthetic methods and reaction schemes described herein, or using other reagents and conventional methods known to persons of ordinary skill in the art. For instance, representative syntheses of compounds of the present disclosure are described in the schemes below, and the particular examples that follow.

### EXAMPLES

### Abbreviations

Certain abbreviations and acronyms are used in describing the experimental details. Although most of these would be understood by one skilled in the art, Table 1 contains a list of many of these abbreviations and acronyms.

TABLE 1
Abbreviations

| Abbreviation | Meaning |
|---|---|
| ° C. | degree(s) Celsius |
| aq | aqueous |
| Boc | tert-butoxycarbonyl |
| Bn | benzyl |
| br s | broad singlet |
| Cbz | benzyloxycarbonyl |
| d | doublet |
| Dₘₐₓ | maximal degradation |
| DC₅₀ | half-maximal degradation concentration |
| DCM | dichloromethane |
| dd | doublet of doublets |
| ddd | doublet of doublet of doublets |
| ddt | doublet of doublet of triplets |
| Dess-Martin | 3-oxo-1λ⁵,2-benziodoxole-1,1,1(3H)-triyl triacetate |
| Periodinane | |
| DMB | 2,4-dimethoxybenzyl |
| DMSO | dimethyl sulfoxide |
| dt | doublet of triplets |
| Et | ethyl |
| EtOAc | ethyl acetate |
| g | gram(s) |
| h | hour(s) |
| HPLC | high-performance liquid chromatography |
| Hz | Hertz |
| ⁱPr | isopropyl |
| IPA or iPrOH | isopropyl alcohol |
| J | coupling constant |
| LCMS | liquid chromatography mass spectrometry |
| m | multiplet |
| M | molarity |
| Me | methyl |
| MeCN | acetonitrile |
| mg | milligram(s) |
| MHz | megahertz |
| min | minute(s) |
| mL | milliliter(s) |
| mm | millimeter(s) |
| mmol | millimole(s) |
| MOMO or OMOM | methoxymethoxy |
| MPa | megapascals |
| MPLC | medium pressure liquid chromatography |
| NMR | nuclear magnetic resonance |
| OSEM | 2-(trimethylsilyl)ethoxymethoxy |
| OTf | trifluoromethanesulfonate |
| Ph | phenyl |
| prep-HPLC | preparative high performance liquid chromatography |
| prep-MPLC | preparative medium pressure liquid chromatography |
| qd | quartet of doublets |
| RP-HPLC | reversed phase high performance liquid chromatography |
| rt or RT | room temperature |
| s | singlet |
| sat. | saturated |
| SEM | 2-(trimethylsilyl)ethoxymethyl |
| SFC | supercritical fluid chromatography |
| t | triplet |
| TBS | tert-butyldimethylsilyl |
| ^{t}Bu | tert-butyl |
| td | triplet of doublets |
| TFA | trifluoroacetic acid |
| TIPS | triisopropylsilyl |
| TMS | trimethylsilyl |
| tt | triplet of triplets |
| v/v | volume/volume |
| wt | weight |
| δ | parts per million referenced to residual non-deuterated solvent peak |
| * | * next to a chiral center indicates that the stereochemistry at the chiral center |
| | is assigned arbitrarily, and the specific configuration (R or S) is not known. |

### X. Intermediates

### Intermediate 1-1.

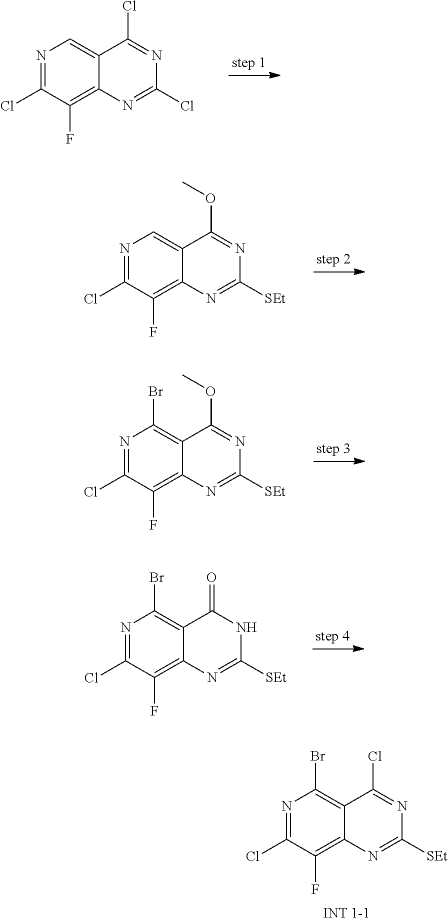

Step 1: 7-Chloro-2-(ethylthio)-8-fluoro-4-methoxypyrido[4,3-d]pyrimidine. Sodium methoxide solution (25% wt in methanol, 20 mmol) was added over 15 min via syringe pump to a vigorously stirred solution of 2,4,7-trichloro-8-fluoropyrido[4,3-d]pyrimidine (19.8 mmol) in 2-methyltetrahydrofuran (70 mL) at −20° C. After 11 min, ethanethiol (59.5 mmol) was added over 1 min via syringe. After 1 min, N,N-diisopropylethylamine (63.5 mmol) was added over 2 min via syringe. After 11 min, the resulting mixture was warmed to room temperature. After 20 min, the resulting mixture was heated to 70° C. After 22 h, the resulting mixture was cooled to room temperature, and citric acid (3.0 g), diethyl ether (200 mL), and ethyl acetate (25 mL) were added sequentially. The organic layer was washed with water (200 mL), was dried over anhydrous magnesium sulfate, was filtered, and was concentrated under reduced pressure. The residue was purified by flash column chromatography on silica gel (0% to 11% ethyl acetate in hexanes) to afford the title compound. LCMS: 274.0.

Step 2: 5-Bromo-7-chloro-2-(ethylthio)-8-fluoro-4-methoxypyrido[4,3-d]pyrimidine. 2,2,6,6-Tetramethylpiperidinylmagnesium chloride lithium chloride complex solution (1.0 M in tetrahydrofuran, 14 mmol) was added over 20 min via syringe pump to a vigorously stirred solution of 7-chloro-2-(ethylthio)-8-fluoro-4-methoxypyrido[4,3-d]pyrimidine (3.65 mmol) in tetrahydrofuran (3.0 mL) at 0° C. After 60 min, a solution of 1,2-dibromo-1,1,2,2-tetrachloroethane (14.6 mmol) in tetrahydrofuran (8.0 mL) was added via syringe. After 120 min, citric acid (5.0 g), diethyl ether (200 mL), and ethyl acetate (25 mL) were added sequentially. The organic layer was washed with water (2×150 mL), was dried over anhydrous magnesium sulfate, was filtered, and was concentrated under reduced pressure. The residue was purified by flash column chromatography on silica gel (0% to 5% ethyl acetate in hexanes) to give the title compound. LCMS: 351.9.

Step 3: 5-Bromo-7-chloro-2-(ethylthio)-8-fluoropyrido[4,3-d]pyrimidin-4(3H)-one. Sodium iodide (1.90 g, 12.7 mmol) was added to a vigorously stirred solution of 5-bromo-7-chloro-2-(ethylthio)-8-fluoro-4-methoxypyrido[4,3-d]pyrimidine (895 mg, 2.54 mmol) in acetic acid (12.0 mL) at room temperature, and the resulting mixture was heated to 80° C. After 2.5 h, the resulting mixture was cooled to room temperature, and ethyl acetate (100 mL) and aqueous sodium thiosulfate solution (1.0 M, 2.0 mL) were added sequentially. The organic layer was washed with water (100 mL), was dried over anhydrous magnesium sulfate, was filtered, and was concentrated under reduced pressure to give the title compound. LCMS: 337.9.

Step 4: 5-Bromo-4,7-dichloro-2-(ethylthio)-8-fluoropyrido[4,3-d]pyrimidine (INT 1-1). N,N-Diisopropylethylamine (5.08 mmol) was added via syringe to a mixture of 5-bromo-7-chloro-2-(ethylthio)-8-fluoropyrido[4,3-d]pyrimidin-4(3H)-one (2.48 mmol) and phosphorous(V) oxychloride (10 mL) at room temperature, and the resulting mixture was stirred at rt for 15 min before it was concentrated under reduced pressure. The residue was purified by flash column chromatography on silica gel (0 to 13% ethyl acetate in hexanes) to give the title compound. LCMS: 357.9.

### Intermediate 1-2.

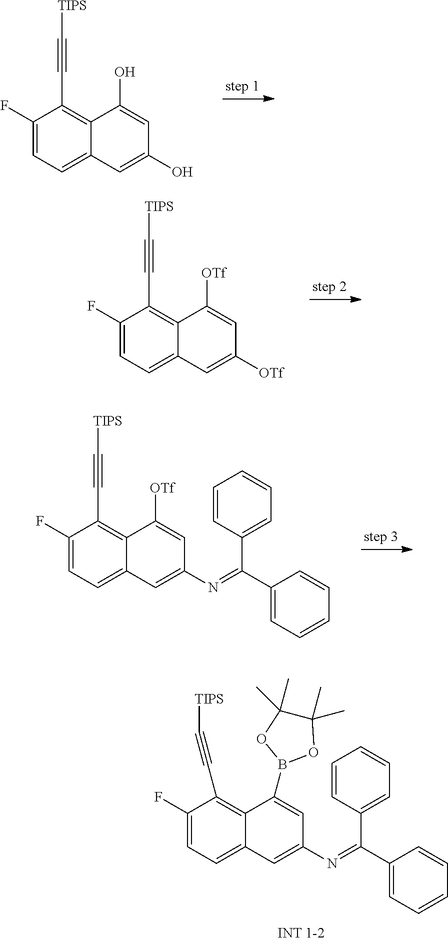

Step 1: 7-Fluoro-8-((triisopropylsilyl)ethynyl)naphthalene-1,3-diyl bis(trifluoromethanesulfonate). To a solution of 7-fluoro-8-((triisopropylsilyl)ethynyl)naphthalene-1,3-diol (61.4 mmol) in dichloromethane (154 mL) at 0° C. were added sequentially N,N-diisopropylethylamine (245 mmol) and trifluoromethanesulfonic anhydride (129 mmol). Upon completion, the reaction was partitioned between water and dichloromethane. The organic fraction was washed with brine, then dried over sodium sulfate and concentrated under reduced pressure to afford the title compound which was used without further purification. ¹H NMR (CDCl₃, 400 MHz) δ 7.95-7.75 (m, 2H), 7.55-7.45 (m, 2H), 1.40-1.15 (m, 21H).

Step 2: 3-((Diphenylmethylene)amino)-7-fluoro-8-((triisopropylsilyl)ethynyl)naphthalen-1-yl trifluoromethanesulfonate. To a mixture of 7-fluoro-8-((triisopropylsilyl)ethynyl)naphthalene-1,3-diyl bis(trifluoromethanesulfonate) (41.8 mmol), diphenylmethanimine (45.9 mmol), and toluene (260 mL) were added Cs₂CO₃ (125 mmol), (9,9-dimethyl-9H-xanthene-4,5-diyl)bis(diphenylphosphane) (8.35 mmol) and tris(dibenzylideneacetone)dipalladium(0). The mixture was evacuated and backfilled with nitrogen three times, then heated to 100° C. under nitrogen. Upon completion, volatiles were removed under reduced pressure and the resulting residue was purified by silica gel chromatography eluting with petroleum ether/ethyl acetate. Fractions containing the product were pooled and concentrated under reduced pressure to afford the title compound.

Step 3: N-(6-fluoro-4-(4,4,5,5-tetramethyl-1,3,2-dioxaborolan-2-yl)-5-((triisopropylsilyl)ethynyl)naphthalen-2-yl)-1,1-diphenylmethanimine (INT 1-2). 3-((diphenylmethylene)amino)-7-fluoro-8-((triisopropylsilyl)ethynyl)naphthalen-1-yl trifluoromethanesulfonate (28.2 mmol), bis(pinacolato)diboron (61.2 mmol), potassium acetate (61.2 mmol), and [1,1′-bis(diphenylphosphino)ferrocene]dichloropalladium(II) (3.06 mmol), were combined in a flask and diluted with toluene (200 mL). The mixture was evacuated and backfilled with N₂ three times, then heated to 110° C. under nitrogen. Upon completion, volatiles were removed under reduced pressure and the resulting residue was purified by silica gel chromatography eluting with petroleum ether/ethyl acetate. Fractions containing the product were pooled and concentrated under reduced pressure to afford the title compound. ¹H NMR (DMSO-d₆, 400 MHz) δ 7.87-7.80 (m, 1H), 7.74-7.68 (m, 2H), 7.62-7.55 (m, 1H), 7.53-7.47 (m, 2H), 7.47-7.39 (m, 1H), 7.35-7.27 (m, 4H), 7.23-7.18 (m, 2H), 7.15-7.10 (m, 1H), 1.26 (s, 12H), 1.15-1.07 (m, 21H). LCMS: 632.

### Intermediate 1-3.

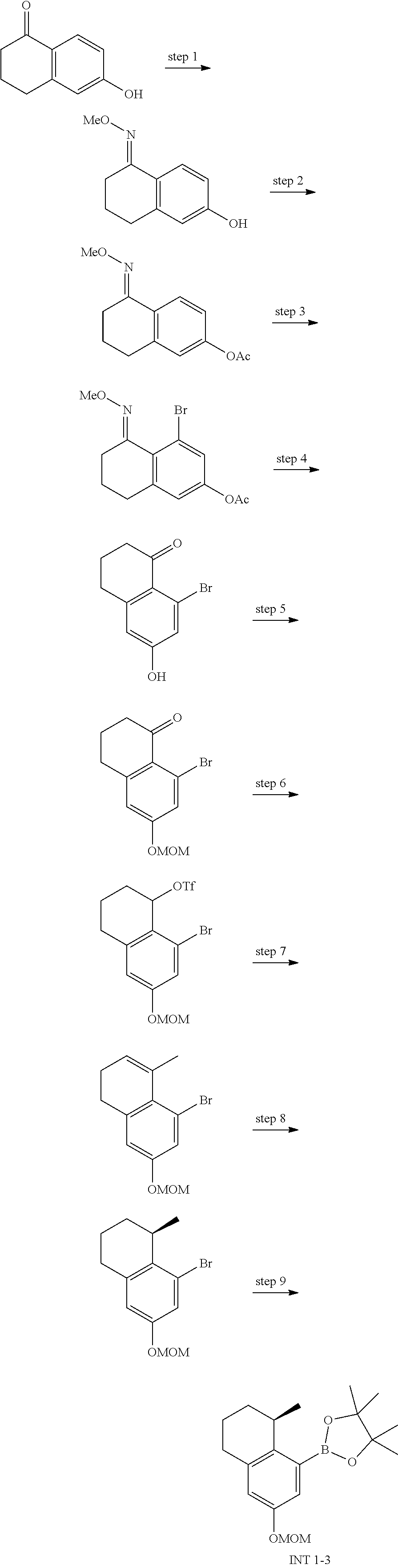

Step 1: 6-Hydroxy-3,4-dihydronaphthalen-1(2H)-one O-methyl oxime. To a stirred mixture of 6-hydroxy-3,4-dihydronaphthalen-1(2H)-one (0.86 mol) and O-methylhydroxylamine hydrochloride (1.3 mol) in MeCN (1.4 L) was added pyridine (1.3 mol) at room temperature. The resulting mixture was stirred at room temperature for 2 h. The reaction was quenched with water and extracted with MTBE (3×1 L). The combined organic layers were washed with brine (2×500 mL), dried over anhydrous sodium sulfate. After filtration, the filtrate was concentrated under reduced pressure to yield the title compound which was used in the next step without further purification. ¹H NMR (CDCl₃, 400 MHz) δ 7.86 (d, J=8.6 Hz, 1H), 6.67 (dd, J=8.6, 2.7 Hz, 1H), 6.59 (d, J=2.7 Hz, 1H), 3.95 (s, 3H), 2.71 (d, J=6.6 Hz, 2H), 2.69-2.64 (m, 2H), 1.87-1.76 (m, 2H).

Step 2: 5-(methoxyimino)-5,6,7,8-tetrahydronaphthalen-2-yl acetate. To a stirred mixture of 5-(methoxyimino)-7,8-dihydro-6H-naphthalen-2-ol (0.81 mol) and triethylamine (1.6 mol) in MTBE (2 L) was added acetyl chloride (1.2 mol) at 0° C. under nitrogen atmosphere. The resulting mixture was stirred at room temperature for 3 h, then quenched with water at 0° C. The resulting mixture was extracted with MTBE (3×1 L). The combined organic layers were washed with brine (2×500 mL), dried over anhydrous sodium sulfate. After filtration, the filtrate was concentrated under reduced pressure. The residue was purified by silica gel chromatography eluting with ethyl acetate/hexanes. Fractions containing the product were pooled and concentrated under reduced pressure to yield the title compound. ¹H NMR (CDCl₃, 400 MHz) δ 7.99 (d, J=8.6 Hz, 1H), 6.94-6.85 (m, 2H), 3.97 (s, 3H), 2.72 (q, J=6.5 Hz, 4H), 2.29 (s, 3H), 1.90-1.79 (m, 2H).

Step 3: 4-Bromo-5-(methoxyimino)-5,6,7,8-tetrahydronaphthalen-2-yl acetate. To a stirred mixture of 5-(methoxyimino)-7,8-dihydro-6H-naphthalen-2-yl acetate (0.47 mol) and NBS (0.5 mol) in acetic acid (1.1 L) was added palladium(II) acetate (47.2 mmol) at room temperature under nitrogen atmosphere. The resulting mixture was stirred at 60° C. for 5 h under nitrogen atmosphere. The resulting mixture was diluted with water and extracted with ethyl acetate (3×1 L). The combined organic layers were washed with NaHCO₃ (aq) and dried over anhydrous sodium sulfate. The residue was purified by silica gel chromatography eluting with ethyl acetate/hexanes. Fractions containing the product were pooled and concentrated under reduced pressure to yield the title compound. ¹H NMR (CDCl₃, 300 MHz): δ 7.32 (d, J=2.4 Hz, 1H), 6.91 (dt, J=2.5, 0.8 Hz, 1H), 4.05 (s, 3H), 2.78 (t, J=6.9 Hz, 2H), 2.70-2.60 (m, 2H), 2.31 (s, 3H), 1.86-1.71 (m, 2H).

Step 4: 8-Bromo-6-hydroxy-3,4-dihydronaphthalen-1(2H)-one. To a stirred solution of 4-bromo-5-(methoxyimino)-7,8-dihydro-6H-naphthalen-2-yl acetate (0.33 mol) in 1,4-dioxane (500 mL) was added HCl (2 M, 500 mL) at room temperature. The resulting mixture was stirred at 100° C. for 6 h. The resulting mixture was then diluted with water extracted with ethyl acetate. The combined organic layers were washed with brine (2×500 mL), dried over anhydrous sodium sulfate. After filtration, the filtrate was concentrated under reduced pressure. The residue was purified by silica gel chromatography eluting with ethyl acetate/hexanes. Fractions containing the product were pooled and concentrated under reduced pressure to yield the title compound. ¹H NMR (CDCl₃, 400 MHz) δ 7.08 (d, J=2.5 Hz, 1H), 6.68 (d, J=2.5 Hz, 1H), 2.92 (t, J=6.1 Hz, 2H), 2.65 (t, J=6.6 Hz, 2H), 2.07 (p, J=6.5 Hz, 2H).

Step 5: 8-bromo-6-(methoxymethoxy)-3,4-dihydronaphthalen-1(2H)-one. To a stirred solution of 8-bromo-6-hydroxy-3,4-dihydro-2H-naphthalen-1-one (311 mmol) and dimethoxymethane (3.1 mol) in ethyl acetate (400 mL) was added 2,4,6-tripropyl-2,4,6-trioxo-1,3,5,2,4,6-trioxatriphosphorinane (622 mmol, 50% in ethyl acetate) at room temperature. The resulting mixture was stirred at 50° C. for 16 h. The reaction was quenched with sat. potassium carbonate (aq.) at room temperature. The resulting mixture was then diluted with water extracted with ethyl acetate. The combined organic layers were washed with brine (2×500 mL), dried over anhydrous sodium sulfate. After filtration, the filtrate was concentrated under reduced pressure. The residue was purified by silica gel chromatography eluting with ethyl acetate/hexanes. Fractions containing the product were pooled and concentrated under reduced pressure to yield the title compound. ¹H NMR (CDCl₃, 400 MHz): δ 7.24 (d, J=2.5 Hz, 1H), 6.85 (d, J=2.4 Hz, 1H), 5.20 (s, 2H), 3.48 (s, 3H), 2.94 (t, J=6.1 Hz, 2H), 2.65 (t, J=6.6 Hz, 2H), 2.07 (p, J=6.4 Hz, 2H).

Step 6: 8-Bromo-6-(methoxymethoxy)-3,4-dihydronaphthalen-1-yl trifluoromethanesulfonate. To a stirred mixture of 8-bromo-6-(methoxymethoxy)-3,4-dihydro-2H-naphthalen-1-one (105 mmol) and N-phenyl-bis(trifluoromethanesulfonimide) (116 mmol) in THE (450 mL) was added KHMDS (116 mL, 1 M in THF) at 0° C. under nitrogen atmosphere. The resulting mixture was stirred at 0° C. for 0.5 h under nitrogen atmosphere. The reaction was quenched with water at 0° C. The resulting mixture was diluted with water and extracted with ethyl acetate (3×1 L). The combined organic layers were washed with NaHCO₃ (aq) and dried over anhydrous sodium sulfate. The residue was purified by silica gel chromatography eluting with ethyl acetate/hexanes. Fractions containing the product were pooled and concentrated under reduced pressure to yield the title compound. ¹H NMR (CDCl₃, 400 MHz) δ 7.45-7.27 (m, 1H), 7.18 (d, J=2.5 Hz, 1H), 6.85 (d, J=2.4 Hz, 1H), 6.14 (t, J=5.3 Hz, 1H), 5.16 (s, 2H), 3.48 (s, 3H), 2.77 (dd, J=8.8, 6.3 Hz, 2H), 2.38-2.29 (m, 2H).

Step 7: 5-Bromo-7-(methoxymethoxy)-4-methyl-1,2-dihydronaphthalene. To a stirred mixture of 8-bromo-6-(methoxymethoxy)-3,4-dihydronaphthalen-1-yl trifluoromethanesulfonate (84 mmol) and Fe(acac)₃ (4.2 mmol) in THE (520 mL) and NMP (35 mL) was added methylmagnesium bromide solution (52.6 mL, 3 M in Et₂O) at 0° C. under nitrogen atmosphere. The resulting mixture was stirred at room temperature for 0.5 h under nitrogen atmosphere. The reaction was quenched with water at 0° C. The resulting mixture was diluted with water and extracted with ethyl acetate (3×1 L). The combined organic layers were washed with NaHCO₃ (aq) and dried over anhydrous sodium sulfate. The residue was purified by silica gel chromatography eluting with ethyl acetate/hexanes. Fractions containing the product were pooled and concentrated under reduced pressure to yield the title compound. ¹H NMR (CDCl₃, 400 MHz) δ 7.16 (d, J=2.6 Hz, 1H), 6.82 (d, J=2.6 Hz, 1H), 6.02-5.95 (m, 1H), 5.14 (s, 2H), 3.47 (s, 3H), 2.63 (dd, J=8.7, 6.2 Hz, 2H), 2.33 (q, J=1.6 Hz, 3H), 2.10-2.00 (m, 2H).

Step 8: (R)-8-Bromo-6-(methoxymethoxy)-1-methyl-1,2,3,4-tetrahydronaphthalene. To a stirred solution of (R,R)-[2-(4′-i-Propyloxazolin-2′-yl)ferrocenyl]diphenylphosphine (3.4 mmol) in dichloroethane (20 mL) was added bis(cyclooctadiene)iridium(I) tetrakis(3,5-bis(trifluoromethyl)phenyl)borate (3.4 mmol) at room temperature under nitrogen atmosphere. The resulting mixture was stirred at 70° C. for 4 h under nitrogen atmosphere. The mixture was allowed to cool down to room temperature. To the above mixture were added 5-bromo-7-(methoxymethoxy)-4-methyl-1,2-dihydronaphthalene (67 mmol) at room temperature. The resulting mixture was stirred at room temperature for 16 h under 250 psi hydrogen atmosphere. The resulting mixture was concentrated under reduced pressure. The residue was first purified by silica gel column chromatography eluting with ethyl acetate/hexanes then by preparative HPLC using an (S,S)-Whelk-O1 column with a mobile phase of isopropanol containing 20 mM NH₃ at a flow rate of 2 mL/min to yield the title compound. [α]_{D}²²=+4.0° (c 0.3, MeOH)) ¹H NMR (400 MHz, Chloroform-d) δ 7.13-7.08 (m, 1H), 6.71 (dd, J=2.5, 1.2 Hz, 1H), 5.11 (s, 2H), 3.46 (s, 3H), 3.16 (qq, J=6.3, 3.4 Hz, 1H), 2.83-2.64 (m, 2H), 1.93-1.67 (m, 4H), 1.21 (d, J=6.9 Hz, 3H).

Step 9: (R)-2-(3-(Methoxymethoxy)-8-methyl-5,6,7,8-tetrahydronaphthalen-1-yl)-4,4,5,5-tetramethyl-1,3,2-dioxaborolane (INT 1-3). To a stirred mixture of (8R)-1-bromo-3-(methoxymethoxy)-8-methyl-5,6,7,8-tetrahydronaphthalene (12.6 mmol) and bis(pinacolato)diboron (18.9 mmol) in 1,4-dioxane (70 mL) were added potassium acetate (37.9 mmol) and 1,1′-bis(diphenylphosphino)ferrocene]dichloropalladium(II) (1.26 mmol) at room temperature under nitrogen atmosphere. The resulting mixture was stirred at 100° C. for 10 h under nitrogen atmosphere. The resulting mixture was concentrated under reduced pressure. The residue was purified by silica gel chromatography eluting with ethyl acetate/hexanes. Fractions containing the product were pooled and concentrated under reduced pressure to yield the title compound. ¹H NMR (CDCl₃, 400 MHz) δ 7.24 (d, J=2.9 Hz, 1H), 6.80 (d, J=2.8 Hz, 1H), 5.18-5.10 (m, 2H), 3.62 (d, J=17.5 Hz, 1H), 3.47 (s, 3H), 2.74 (d, J=5.6 Hz, 2H), 1.88-1.77 (m, 2H), 1.75-1.64 (m, 2H), 1.34 (d, J=2.1 Hz, 12H), 1.17 (d, J=7.0 Hz, 3H).

### Intermediates 1-4-1-5.

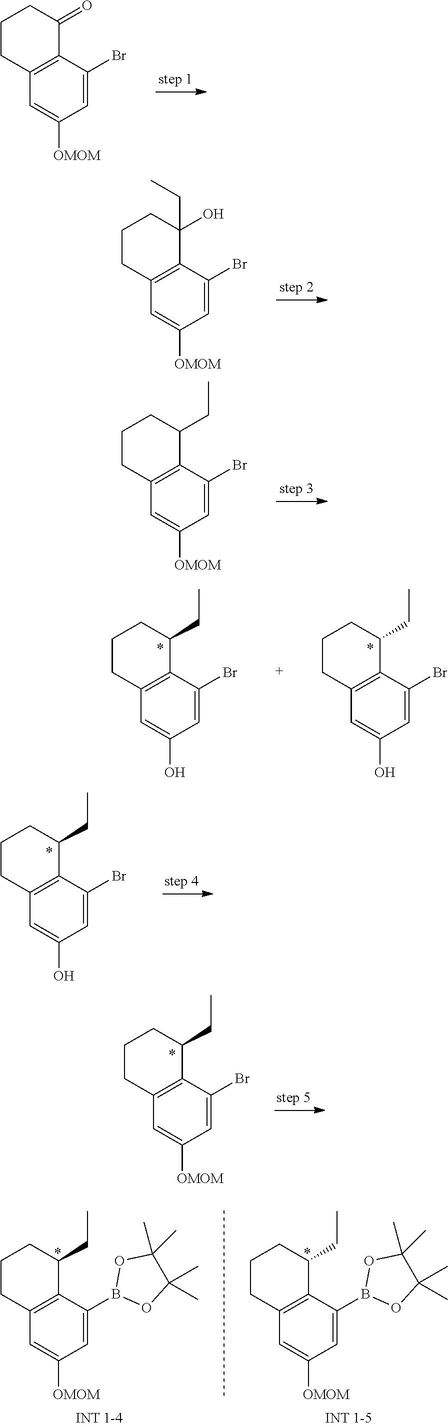

Step 1: 8-Bromo-1-ethyl-6-(methoxymethoxy)-1,2,3,4-tetrahydronaphthalen-1-ol. To a stirred solution of 8-bromo-6-(methoxymethoxy)-3,4-dihydronaphthalen-1(2H)-one (42.3 mmol) in THE (120 mL) at 0° C. under N₂ atmosphere was added lanthanum(III) chloride bis(lithium chloride) complex solution (70.4 mL, 42.3 mmol, 0.6 M in THF) dropwise. The reaction mixture was stirred for 10 min at 0° C. Ethylmagnesium bromide (16.9 mL, 50.7 mmol, 3M in diethyl ether) was added dropwise and the reaction mixture was stirred at 0° C. for 1 h. Upon completion the reaction mixture was quenched with saturated aqueous NH₄Cl solution and filtered through a pad of celite. The filter cake was rinsed with EtOAc (150 mL), and the organic layer was separated. The aqueous layer was extracted with EtOAc (2×150 mL), and the combined organic layer was washed with brine and dried over anhydrous Na₂SO₄, filtered, and concentrated under reduced pressure. The crude residue was purified by silica gel chromatography eluting with 0-20% EtOAc/petroleum ether to yield the title compound.

Step 2: 8-Bromo-1-ethyl-6-(methoxymethoxy)-1,2,3,4-tetrahydronaphthalene. To a stirred solution of 8-bromo-1-ethyl-6-(methoxymethoxy)-1,2,3,4-tetrahydronaphthalen-1-ol (34.4 mmol) in DCM (110 mL) at −40° C. under nitrogen atmosphere was added triethylsilane (Et₃SiH, 27.8 mL, 171.94 mmol) dropwise, followed by trifluoroacetic acid (TFA, 7.9 mL, 103.2 mmol) dropwise. The reaction mixture was stirred for 2 hours at −40° C. Upon completion, the reaction was quenched with saturated aqueous NH₄Cl and extracted with EtOAc (2×100 mL). The combined organic layers were washed with saturated aqueous NaHCO₃, dried over anhydrous Na₂SO₄, filtered, and concentrated under reduced pressure. The crude residue was purified by silica gel column chromatography, eluting with EtOAc/petroleum ether, to yield the title compound. LCMS: 298.05

Step 3: (R)-4-Bromo-5-ethyl-5,6,7,8-tetrahydronaphthalen-2-ol and (S)-4-bromo-5-ethyl-5,6,7,8-tetrahydronaphthalen-2-ol. To a stirred solution of 8-bromo-1-ethyl-6-(methoxymethoxy)-1,2,3,4-tetrahydronaphthalene (18.5 mmol) in 1,4-dioxane (55 mL) at 0° C. under nitrogen atmosphere was added 4 M HCl in 1,4-dioxane (55 mL) dropwise at 0° C. The reaction mixture was allowed to warm to room temperature and stirred for 3 h. Upon completion, the reaction mixture was concentrated under reduced pressure. The crude residue was dissolved in EtOAc (20 mL), washed with saturated aqueous NaHCO₃, dried over anhydrous Na₂SO₄, filtered, and concentrated under reduced pressure. This mixture was further purified by chiral prep-SFC to yield (R*)-4-bromo-5-ethyl-5,6,7,8-tetrahydronaphthalen-2-ol (peak 1) and (S*)-4-bromo-5-ethyl-5,6,7,8-tetrahydronaphthalen-2-ol (peak 2), with stereochemistry assigned arbitrarily. LCMS: 254.0

Step 4: (R*)-8-Bromo-1-ethyl-6-(methoxymethoxy)-1,2,3,4-tetrahydronaphthalene. To a stirred solution of (R*)-4-bromo-5-ethyl-5,6,7,8-tetrahydronaphthalen-2-ol (Peak 1, 7.1 mmol) in dichloromethane (18 mL) at 0° C. under nitrogen atmosphere was added N,N-diisopropylethylamine (3.7 mL, 21.3 mmol), followed by methoxymethyl chloride (0.8 mL, 10.6 mmol) dropwise at 0° C. The reaction mixture was allowed to warm to room temperature and stirred for 16 h. Upon completion, the reaction mixture was diluted with water and extracted with DCM (2×10 mL). The combined organic layer was dried over anhydrous Na₂SO₄ and concentrated under reduced pressure. The crude residue was purified by silica gel column chromatography, eluting with 0-20% EtOAc/petroleum ether to yield the title compound. LCMS: 298.1

Step 5: (R*)-2-(8-Ethyl-3-(methoxymethoxy)-5,6,7,8-tetrahydronaphthalen-1-yl)-4,4,5,5-tetramethyl-1,3,2-dioxaborolane (INT 1-4). To a stirred solution of (R)-8-bromo-1-ethyl-6-(methoxymethoxy)-1,2,3,4-tetrahydronaphthalene (1.9 g, 6.4 mmol) in toluene (19 mL) was added B₂Pin₂ (2.4 g, 9.6 mmol) & KOAc (1.9 g, 19.1 mmol) under N₂ atmosphere. The mixture was degassed for 5 min using N₂ gas, then Pd(dppf)Cl₂ (0.47 g, 0.64 mmol) was added and the mixture was degassed again for 5 min using N₂ gas. The reaction mixture was then stirred at 100° C. for 16 h. Upon completion, the reaction mixture was filtered through celite, washed with toluene (15 mL), and concentrated under reduced pressure. The crude residue was purified silica gel column chromatography eluting with 0-20% EtOAc/petroleum ether to yield the title compound. LCMS: 346.2

(S*)-2-(8-ethyl-3-(methoxymethoxy)-5,6,7,8-tetrahydronaphthalen-1-yl)-4,4,5,5-tetramethyl-1,3,2-dioxaborolane (INT 1-5) was prepared using the procedures described above, starting with Peak 2 isolated in Step 3. LCMS: 346.2

### Intermediate 1-6.

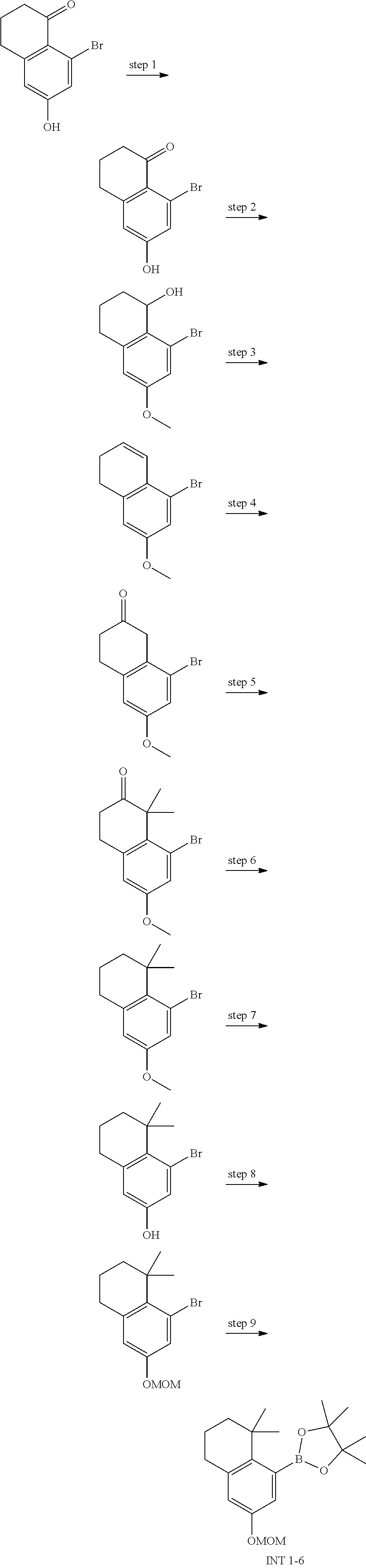

Step 1: 8-Bromo-6-methoxy-3,4-dihydronaphthalen-1(2H)-one. A stirred solution of 8-bromo-6-hydroxy-3,4-dihydronaphthalen-1(2H)-one (40 g, 167 mmol) in DMF (400 mL) was cooled to 0° C., then potassium carbonate (69 g, 500 mmol) was added portion-wise, followed by dropwise addition of methyl iodide (15.6 mL, 250 mmol). The reaction mixture was allowed to warm to room temperature and stirred for 4 h. Upon completion, the reaction mixture was quenched with ice cold water then extracted with EtOAc (2×300 mL). The combined organic layer was washed with brine (2×500 mL), dried over anhydrous Na₂SO₄, filtered and concentrated under reduced pressure. The crude residue was purified by silica gel column chromatography eluting with 0-40% EtOAc/petroleum ether to yield the title compound. LCMS: 255.1

Step 2: 8-Bromo-6-methoxy-1,2,3,4-tetrahydronaphthalen-1-ol. To a stirred solution of 8-bromo-6-methoxy-3,4-dihydronaphthalen-1(2H)-one (36 g, 142 mmol) in EtOH (540 mL) and THE (180 mL) at 0° C. under N₂ atmosphere was added NaBH₄ (6.4 g, 170 mmol). The reaction mixture was then allowed to warm to room temperature and stirred for 6 h. Upon completion, the reaction mixture was concentrated under reduced pressure. The crude residue was dissolved in EtOAc, diluted with water (300 mL), then extracted with EtOAc (200 mL×2). The combined organic layer was washed with brine (300 mL×2) & dried over anhydrous Na₂SO₄, filtered, and concentrated under reduced pressure to afford the title compound which was used in the next step without further purification.

Step 3: 5-Bromo-7-methoxy-1,2-dihydronaphthalene. To a stirred solution of 8-bromo-6-methoxy-1,2,3,4-tetrahydronaphthalen-1-ol (35 g, 137 mmol) in toluene (350 mL) at 0° C. under N₂ atmosphere was added PTSA (350 mg) added portion wise. The reaction mixture was then stirred at 110° C. for 16 h. Upon completion, the reaction mixture was cooled to 0° C. and quenched with sat. NaHCO₃ solution. The mixture was extracted with EtOAc (200 mL×2), and the combined organic layer was dried over anhydrous Na₂SO₄, filtered, and concentrated under reduced pressure. The crude residue was purified by silica gel column chromatography eluting with 0-20% EtOAc/petroleum ether yield the title compound. LCMS: 239.4

Step 4: 8-Bromo-6-methoxy-3,4-dihydronaphthalen-2(1H)-one. To a stirred solution of 5-bromo-7-methoxy-1,2-dihydronaphthalene (25 g, 137 mmol) in DCM (250 mL) at 0° C. under N₂ atmosphere, mCPBA (51 g, 242 mmol) was added portion-wise. The reaction mixture was then allowed to warm to room temperature and stirred for 16 hours. Upon completion, the reaction mixture was cooled to 0° C. and quenched with sat. NaHCO₃ solution. The mixture was then extracted with EtOAc (200 mL×2), the combined organic layer was dried over anhydrous Na₂SO₄, filtered, and concentrated under reduced pressure. The crude residue was dissolved in ethanol (250 ml) & H₂SO₄(25 mL), and was heated to 70° C. and stirred for 3 h. Upon completion, the reaction mixture was quenched with sat. NaHCO₃ solution at 0° C., then extracted with EtOAc (200 mL×2). The combined organic layer was dried over anhydrous Na₂SO₄, filtered, and concentrated under reduced pressure. The crude residue was purified by silica gel column chromatography eluting with 0-20% EtOAc/petroleum ether to yield the title compound. LCMS: 253.0

Step 5: 8-bromo-6-methoxy-1,1-dimethyl-3,4-dihydronaphthalen-2(1H)-one. To a stirred solution of 8-bromo-6-methoxy-3,4-dihydronaphthalen-2(1H)-one (3.7 g, 14.6 mmol) in THE (37 ml) at 0° C. under N₂ atmosphere was added NaH (60% dispersion in mineral oil) (1.5 g, 36.4 mmol) portion-wise. The reaction mixture was then stirred for 20 mins at 0° C. Then, MeI (3.6 mL, 58 mmol) was added at same temperature under N₂ atmosphere. The reaction was allowed to warm to room temperature and stirred for 2 h. Upon completion, the reaction mixture was quenched with ice cold water (200 mL), then extracted with EtOAc (200 mL×2). The combined organic layer was dried over anhydrous Na₂SO₄, filtered, and concentrated under reduced pressure. The crude residue was purified by silica gel column chromatography, eluting with 0-30% EtOAc/petroleum ether to yield the title compound.

Step 6: 8-Bromo-6-methoxy-1,1-dimethyl-1,2,3,4-tetrahydronaphthalene. To a stirred solution of 8-bromo-6-methoxy-1,1-dimethyl-3,4-dihydronaphthalen-2(1H)-one (1.8 g, 6.4 mmol) in ethylene glycol (18 mL) at 0° C. under N₂ atmosphere KOH (3.6 g, 64 mmol) was added portion-wise. After that, hydrazine hydrate (3.1 mL, 64 mmol) was added drop-wise at 0° C. under N₂ atmosphere, then the reaction mixture was heated to 200° C. and stirred for 16 h. Upon completion the reaction mixture was cooled to RT and quenched with ice cold water (100 mL), then extracted with EtOAc (100 mL×2). The combined organic layer was dried over anhydrous Na₂SO₄, filtered, and concentrated under reduced pressure. The crude residue was purified by silica gel column chromatography eluting with 0-30% EtOAc/petroleum ether to yield the title compound.

Step 7: 4-Bromo-5,5-dimethyl-5,6,7,8-tetrahydronaphthalen-2-ol. To a stirred solution of 8-bromo-6-methoxy-1,1-dimethyl-1,2,3,4-tetrahydronaphthalene (1.1 g, 4.3 mmol) in DCM (11 mL) at 0° C. under N₂ atmosphere, 1M BBr₃ in DCM (6.16 mL, 6.16 mmol) was added drop-wise. The reaction mixture was allowed to warm to RT and stirred for 3 h. Upon completion, the reaction mixture was quenched with ice cold water (100 mL), extracted with EtOAc (100 mL×2), and the combined organic layer was dried over anhydrous Na₂SO₄, filtered, and concentrated under reduced pressure yield the title compound which was used in the next step without further purification. LCMS: 254.96

Step 8: 8-Bromo-6-(methoxymethoxy)-1,1-dimethyl-1,2,3,4-tetrahydronaphthalene. To a stirred solution of 4-bromo-5,5-dimethyl-5,6,7,8-tetrahydronaphthalen-2-ol (0.9 g, 3.5 mmol) in DCM (11 mL) at 0° C. under N₂ atmosphere was added DIPEA (1.85 mL, 10.6 mmol) dropwise. The reaction mixture was stirred for 5 min, then MOMCl (0.4 mL, 5.3 mmol) was added dropwise. The reaction mixture was allowed to warm to RT then stirred for 16 h. Upon completion, the reaction mixture was quenched ice cold water (100 mL), and extracted with EtOAc (100 mL×2). The combined organic layer was dried over anhydrous Na₂SO₄, filtered, and concentrated under reduced pressure to yield the title compound which was used in the next step without further purification.

Step 9: 2-(3-(Methoxymethoxy)-8,8-dimethyl-5,6,7,8-tetrahydronaphthalen-1-yl)-4,4,5,5-tetramethyl-1,3,2-dioxaborolane (INT 1-6). To a stirred solution of 8-bromo-6-(methoxymethoxy)-1,1-dimethyl-1,2,3,4-tetrahydronaphthalene (1 g, 3.4 mmol) and B₂pin₂ (1.3 g, 5.1 mmol) in toluene (10 mL) was added KOAc (1 g, 10.2 mmol). The reaction mixture was degassed with argon gas for 5 min, then Pd(dppf)Cl₂ (0.2 g, 0.33 mmol) was added and the mixture was again degassed for 5 mins with argon gas. The reaction mixture was stirred at 100° C. for 16 h. Upon completion, the reaction mixture was filtered through celite, the filtrate was concentrated under reduced pressure. The crude residue was purified by silica gel column chromatography eluting with 0-20% EtOAc/petroleum ether to yield the title compound. LCMS: 347.3

### Intermediate 1-7.

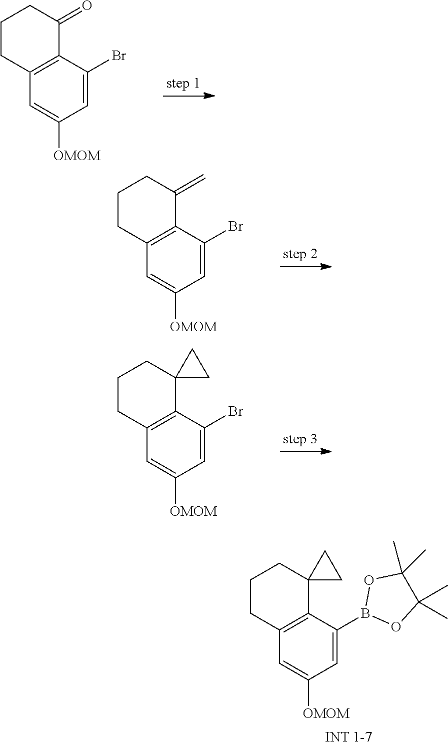

Step 1: 8-Bromo-6-(methoxymethoxy)-1-methylene-1,2,3,4-tetrahydronaphthalene. To a solution of MePPh₃Br (50 g, 140 mmol) in toluene (200 mL) under argon at 0° C. was added portion-wise KOtBu (23.7 g, 925 mmol). The resulting mixture was stirred for 20 min. Then, 8-bromo-6-(methoxymethoxy)-3,4-dihydronaphthalen-1(2H)-one (20 g, 70.2 mmol) in THE (200 mL) was added dropwise at 0° C. The resulting reaction mixture was stirred for 1 h at 0° C. The reaction mixture was allowed to warm to RT and stirred for 3 h. Upon completion, the reaction mixture was quenched with sat. NH₄Cl solution and extracted with MTBE (2×500 mL). The combined organic layer was washed with brine solution (100 mL), dried over Na₂SO₄, filtered, and concentrated under reduced pressure. The crude residue was triturated with n-pentane to yield the title compound which was used in the next step without further purification. LCMS: 283.2

Step 2: 8′-Bromo-6′-(methoxymethoxy)-3′,4′-dihydro-2′H-spiro[cyclopropane-1,1′-naphthalene]. A solution of 1 M diethyl zinc in hexanes (105 mL, 105 mmol) in DCM (74 mL) was cooled to 0° C. Then a solution of trifluoroacetic acid (105 mmol) in DCM (30 mL) was added dropwise. The reaction mixture was stirred for 20 min at 0° C. Then, a solution of diodomethane (28 g, 105 mmol) in DCM (30 mL) was added dropwise. The reaction mixture was stirred for an additional 20 min at same temperature. Then, a solution of 8-bromo-6-(methoxymethoxy)-1-methylene-1,2,3,4-tetrahydronaphthalene (14.8 g, 52.3) in DCM (74 mL) was added to the mixture. The reaction mixture was allowed to warm to room temperature and stirred for 12 h. Upon completion, the reaction mixture was quenched with sat. ammonium chloride solution, and the organic layer was separated. The aqueous layer was extracted with DCM. The combined organic layer was washed with saturated NaHCO₃ (150 mL) and brine solution (50 mL). The organic layer was dried over the Na₂SO₄, filtered, and concentrated under reduced pressure. The crude residue was purified by silica gel column chromatography eluting with 0-10% EtOAc/petroleum ether to yield the title compound.

Step 3: 2-(6′-(Methoxymethoxy)-3′,4′-dihydro-2′H-spiro[cyclopropane-1,1′-naphthalen]-8′-yl)-4,4,5,5-tetramethyl-1,3,2-dioxaborolane (INT 1-7). To a stirred solution of 8′-bromo-6′-(methoxymethoxy)-3′,4′-dihydro-2′H-spiro[cyclopropane-1,1′-naphthalene](5 g, 16.8 mmol) in toluene (50 mL) was added B₂Pin₂ (8.5 g, 33.7 mmol) and KOAc (15 g, 50.5 mmol). The mixture was degassed for 5 min with N₂, then Pd(dppf)Cl₂ (1.2 g, 1.7 mmol) was added, and the mixture was degassed again for 5 min with N₂. The reaction mixture was then stirred at 100° C. for 16 h. Upon completion, the reaction mixture was filtered through celite, which was rinsed with toluene (50 mL). The filtrate was concentrated under reduced pressure. The crude residue was purified by silica gel column chromatography eluting with 0-20% EtOAc/petroleum ether to yield the title compound. LCMS: 344.2

### Intermediate 1-8.

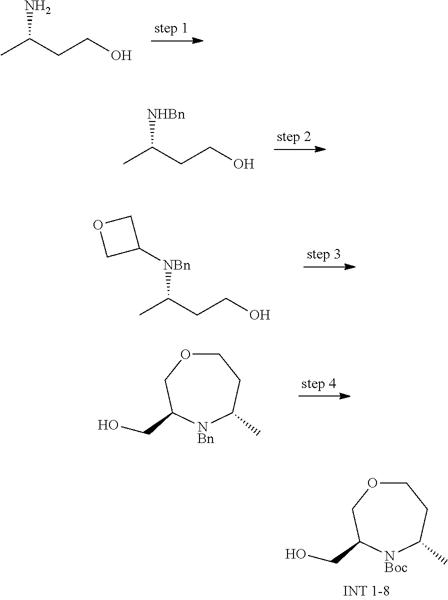

Step 1: (S)-3-(Benzylamino)butan-1-ol. To a solution of (S)-3-aminobutan-1-ol. To a solution of (S)-3-aminobutan-1-ol (50 g, 560 mmol, 1.0 equiv) in methanol (900 mL) was added benzaldehyde (60 g, 560 mmol, 1.0 equiv) at room temperature, and the reaction mixture was stirred for 1 h. Sodium borohydride (21 g, 560 mmol, 1.0 equiv) was then added, and stirring continued at room temperature for 2 h. The mixture was cooled to 0° C., quenched with water (600 mL), and concentrated under reduced pressure. The aqueous phase was extracted with dichloromethane (3×500 mL), and the combined organic layers were washed with brine (200 mL), dried over anhydrous sodium sulfate, and concentrated in vacuo to afford the title compound, which was used directly in the next step without further purification. ¹H NMR (300 MHz, Methanol-d₄) δ 7.37-7.25 (m, 5H), 3.85-3.59 (m, 4H), 2.90-2.84 (m, 1H), 1.81-1.74 (m, 1H), 1.62-1.53 (m, 1H), 1.15 (d, J=6.4 Hz, 3H).

Step 2: (S)-3-(Benzyl(oxetan-3-yl)amino)butan-1-ol. To a solution of (S)-3-(benzylamino)butan-1-ol (96 g, 540 mmol) in toluene (1000 mL) was added 3-oxetanone (42 g, 590 mmol) at room temperature, and the mixture was stirred at 105° C. for 3 h. After cooling to room temperature, sodium triacetoxyborohydride (82 g, 860 mmol, 1.6 equiv) was added, and the mixture was stirred at 35° C. for 16 h. The reaction was cooled again to room temperature, then aqueous sodium carbonate (750 mL, 2 N) was added, and stirring continued for 1 h. After phase separation, the aqueous layer was extracted with ethyl acetate (300 mL), and the combined organic layers were washed with brine (300 mL), dried over anhydrous sodium sulfate, and concentrated in vacuo to afford the title compound, which was used directly in the next step without further purification. ¹H NMR (300 MHz, Methanol-d₄) δ 7.34-7.14 (m, 5H), 4.64 (t, J=6.5 Hz, 1H), 4.53 (t, J=6.9 Hz, 1H), 4.39-4.32 (m, 2H), 4.25-4.20 (m, 1H), 3.90 (d, J=13.9 Hz, 1H), 3.61-3.49 (m, 3H), 2.88-2.75 (m, 1H), 1.82-1.74 (m, 1H), 1.45-1.36 (m, 1H), 0.98 (d, J=6.6 Hz, 3H).

Step 3: ((3S,5S)-4-Benzyl-5-methyl-1,4-oxazepan-3-yl)methanol. To a solution of (S)-3-(benzyl(oxetan-3-yl)amino)butan-1-ol (78.0 g, 332 mmol) in toluene (800 mL) was added dibutyl hydrogen phosphate (77 g, 365 mmol) at room temperature, and the mixture was stirred at 108° C. overnight. The mixture was cooled to room temperature, aqueous sodium carbonate (800 mL, 2N) was added, and it was stirred at room temperature for 1 hour. After separation, the aqueous phase was extracted with ethyl acetate (200 mL). The combined organic layers were washed with brine (200 mL), dried over anhydrous sodium sulfate, and concentrated in vacuo to give a residue, which was purified by silica gel column chromatography (Petroleum ether:EtOAc=10:1˜3:1) to give a mixture. Then the mixture (28.0 g) was purified by SFC (Column: Chrialpak IH-3, 4.6 cm×5.0 cm, 3 μm, 35° C., Mobile Phase A: CO2, Mobile Phase B: MeOH (0.5% 2 mM NH3-MeOH), Flow Rate: 210 mL/min) to give the title compound. ¹H NMR (400 MHz, Methanol-d4): δ 7.41 (d, J=7.4 Hz, 2H), 7.34 (t, J=7.2 Hz, 2H), 7.25 (t, J=7.2 Hz, 1H), 4.11-3.91 (m, 3H), 3.73-3.61 (m, 3H), 3.44-3.31 (m, 3H), 3.00-2.93 (m, 1H), 2.04-1.92 (m, 1H), 1.80-1.71 (m, 1H), 1.28 (d, J=6.8 Hz, 3H).

Step 4: tert-Butyl (3S,5S)-3-(hydroxymethyl)-5-methyl-1,4-oxazepane-4-carboxylate (INT 1-8). To a solution of ((3S,5S)-4-benzyl-5-methyl-1,4-oxazepan-3-yl)methanol (11.0 g, 46.7 mmol) in MeOH (330 mL) was added di-tert-butyl dicarbonate (15.3 g, 70.1 mmol) and 10% palladium hydroxide on carbon (2.2 g, 20% w/w) at room temperature. The mixture was stirred at 40° C. under hydrogen atmosphere for 16 hours. The mixture was filtered, and the filtrate was concentrated. It was purified by silica gel column chromatography (Petroleum ether:EtOAc=50:1˜10:1) to give the title compound. ¹H NMR (300 MHz, CDCl₃): δ 4.20 (brs, 1H), 3.89-3.74 (m, 5H), 3.68-3.61 (m, 1H), 3.50-3.43 (m, 1H), 2.11-2.00 (m, 1H), 1.75-1.64 (m, 1H), 1.48 (s, 9H), 1.20 (d, J=6.6 Hz, 3H).

### Intermediate 1-9.

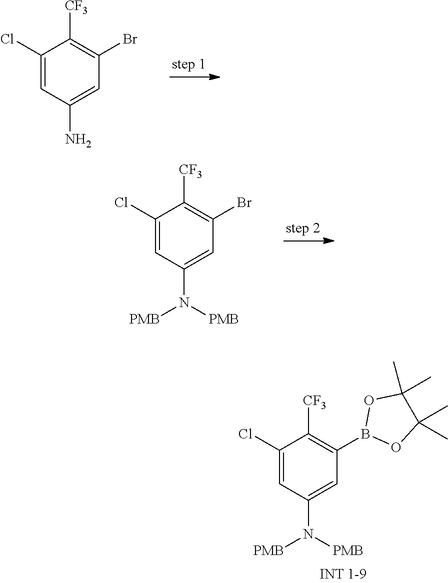

Step 1: 3-Bromo-5-chloro-N,N-bis[(4-methoxyphenyl)methyl]-4-(trifluoromethyl)aniline: To a solution of 3-bromo-5-chloro-4-(trifluoromethyl)aniline (3.6 mmol) in N,N-dimethylformamide (4 mL) was added 4-methoxybenzyl chloride (8.0 mmol), potassium iodide (2.2 mmol), and sodium carbonate (9.1 mmol). The resulting mixture was then stirred at 90° C. for 5 hours. The reaction mixture was then cooled to room temperature, quenched with water (15 mL) and extracted with ethyl acetate (3×15 mL). The combined organic phase was collected, dried over magnesium sulfate, filtered, and concentrated under reduced pressure to afford a crude residue. To the residue was then added ethanol (15 mL) and the resulting precipitate was filtered off as the title compound. LCMS: 514.0.

Step 2: 3-Chloro-N,N-bis[(4-methoxyphenyl)methyl]-5-(4,4,5,5-tetramethyl-1,3,2-dioxaborolan-2-yl)-4-(trifluoromethyl)aniline (INT 1-9): To a solution of 3-bromo-5-chloro-N,N-bis[(4-methoxyphenyl)methyl]-4-(trifluoromethyl)aniline (3.6 mmol) in 1,4-dioxane (71 mL) was added potassium acetate (11 mmol), bis(pinacolato)diboron (8.9 mmol), and (1,1′-Bis(diphenylphosphino)ferrocene)dichloropalladium(II) (0.04 mmol). The resulting mixture was then degassed with argon for 1 minute and then stirred at 100° C. for 16 hours. The mixture was then cooled to room temperature, filtered through Celite, and concentrated under reduced pressure to afford a crude residue. The residue was then purified via flash column chromatography on silica gel eluting with ethyl acetate and hexanes to afford the title compound. LCSM: 562.1.

### Intermediate 2-1.

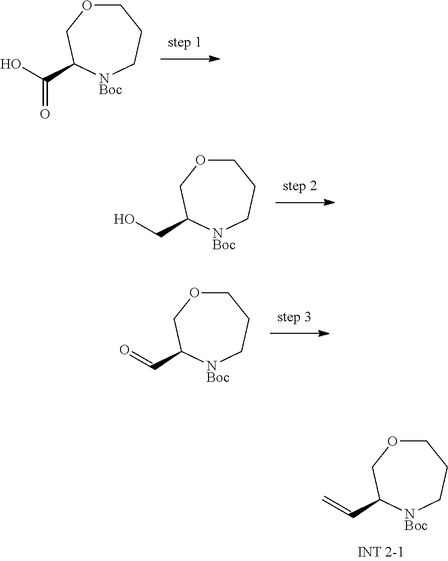 [SMILES: CC(C)(C)OC(=O)N1CCCOC[C@@H]1C(=O)O ; CC(C)(C)OC(=O)N1CCCOC[C@@H]1CO ; CC(C)(C)OC(=O)N1CCCOC[C@@H]1C=O ; C=C[C@H]1COCCCN1C(=O)OC(C)(C)C]

Step 1: tert-Butyl (S)-3-(hydroxymethyl)-1,4-oxazepane-4-carboxylate. To a solution of (R)-4-(tert-butoxycarbonyl)-1,4-oxazepane-3-carboxylic acid (10.2 mmol) in anhydrous tetrahydrofuran (25 mL) at −78° C. was added lithium aluminium hydride (10.2 mL, 2M) in tetrahydrofuran. The reaction mixture was stirred at 0° C. for 1 hour. The mixture was diluted with diethyl ether (20 ml), followed by water (0.78 mL), and an aqueous sodium hydroxide solution (2.4 mL, 15 wt. %). The mixture was then allowed to warm up to room temperature and left stirring for 15 minutes. Anhydrous magnesium sulfate was then added with continued stirring for 15 minutes. Filtration was performed to remove the salts, then the filtrate was concentrated under reduced pressure and the crude title compound was taken forward without further purification.

Step 2: tert-Butyl (R)-3-formyl-1,4-oxazepane-4-carboxylate. To the solution of tert-butyl (S)-3-(hydroxymethyl)-1,4-oxazepane-4-carboxylate (8.6 mmol) in dichloromethane (10 ml) at 0° C. was added Dess-Martin Periodinane (10 mmol). The mixture was stirred at room temperature for 3 hours, then saturated sodium bicarbonate solution (15 ml) was added to the mixture. The mixture was extracted with ethyl acetate. The combined organic layers were dried over anhydrous sodium sulfate, and filtered and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 9.68-9.54 (m, 1H), 4.61-4.24 (m, 1H), 4.13 (ddd, J=32.9, 13.4, 6.6 Hz, 1H), 4.04-3.85 (m, 2H), 3.82-3.50 (m, 3H), 1.96-1.78 (m, 2H), 1.48 (d, J=24.7 Hz, 9H).

Step 3: tert-butyl (S)-3-vinyl-1,4-oxazepane-4-carboxylate (INT 2-1). To a suspension of methyltriphenylphosphonium bromide (6.4 mmol) in tetrahydrofuran (10 ml) at room temperature was added a 1.0 M solution of potassium bis(trimethylsilyl)amide (6.1 ml) dropwise to afford a suspension. The mixture was stirred for 1 hour at room temperature and was cooled to −78° C. whereupon a solution of tert-butyl (R)-3-formyl-1,4-oxazepane-4-carboxylate (3.1 mmol) in tetrahydrofuran (2 ml) was added dropwise. The resulting mixture was allowed to gradually warm to room temperature and stir for 3 hours. The mixture was quenched with methanol and stirred for 15 minutes. Saturated aqueous ammonium chloride solution (10 ml) was added, and the mixture was extracted with ethyl acetate (2×25 ml). The combined organic phase was washed with brine and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 5.84-5.57 (m, 1H), 5.16 (q, J=9.4, 8.4 Hz, 2H), 4.75 (d, J=70.6 Hz, 1H), 4.08-3.79 (m, 2H), 3.65-3.31 (m, 2H), 3.07 (t, J=12.9 Hz, 1H), 2.01-1.53 (m, 3H), 1.48 (d, J=10.2 Hz, 9H).

The following intermediates were made in a similar fashion to INT 2-1 and are shown below in Table 2A. To prepare the below intermediates, different reagents/starting materials were used than some of those described in the steps toward INT 2-1 and are noted in the last column of Table 2A—“Changes to Procedure: Different Reagents/Starting Materials”. A person of ordinary skill in the art will readily recognize which reagents/starting materials used for the synthesis of INT 2-1 were replaced with the different reagents/starting materials noted below.

TABLE 2A
Intermediates

| | | | Changes to |
|---|---|---|---|
| | | | Procedure: |
| | | | Different |
| Inter- | | | Reagents/Starting |
| mediate | Structure | NMR | Materials |
| INT 2-2 | 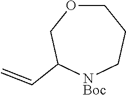 [SMILES: C=CC1COCCCN1C(=O)OC(C)(C)C] | ¹H NMR (400 MHz, Chloroform-d) δ 5.68 (d, J = 14.9 Hz, 1H), 5.16 (d, J = 10.0 Hz, 2H), 4.75 (d, J = 70.3 Hz, 1H), 4.30-3.76 (m, 2H), 3.74-3.24 (m, 2H), 3.07 (t, J = 13.1 Hz, 1H), 1.93 (s, 1H), 1.84-1.51 (m, 2H), 1.55-1.45 (m, 9H). | 4-(tert- butoxycarbonyl)- 1,4-oxazepane-3- carboxylic acid in step 1 |
| INT 2-3 | 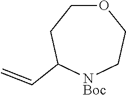 [SMILES: C=CC1CCOCCN1C(=O)OC(C)(C)C] | ¹H NMR (400 MHz, Chloroform-d) δ 5.79 (td, J = 16.5, 8.4 Hz, 1H), 5.15-5.02 (m, 2H), 4.62 (d, J = 90.5 Hz, 1H), 4.14-3.70 (m, 3H), 3.48 (p, J = 9.9, 9.3 Hz, 2H), 3.06 (ddd, J = 15.2, 10.3, 1.5 Hz, 1H), 2.18 (ddd, J = 22.8, 15.1, 7.3 Hz, 1H), 1.86 (dt, J = 15.6, 10.4 Hz, 1H), 1.49 (d, J = 6.8 Hz, 9H). | 4-(tert-butyl) 5- methyl 1,4- oxazepane-4,5- dicarboxylate in step 1 |
| INT 2-4 | 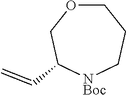 [SMILES: C=C[C@@H]1COCCCN1C(=O)OC(C)(C)C] | ¹H NMR (400 MHz, Chloroform-d) δ 5.65 (d, J = 18.8 Hz, 1H), 5.17 (q, J = 10.5, 7.4 Hz, 2H), 4.76 (d, J = 70.5 Hz, 1H), 4.28- 3.80 (m, 2H), 3.67-3.30 (m, 2H), 3.07 (t, J = 13.1 Hz, 1H), 1.93 (s, 1H), 1.79-1.65 (m, 2H), 1.48 (d, J = 11.6 Hz, 9H). | (S)-4-(tert- butoxycarbonyl)- 1,4-oxazepane-3- carboxylic acid in step 1 |
| INT 2-5 | 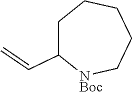 [SMILES: C=CC1CCCCCN1C(=O)OC(C)(C)C] | ¹H NMR (400 MHz, Chloroform-d) δ 5.84- 5.63 (m, 1H), 5.09-4.91 (m, 2H), 4.66- 4.32 (m, 1H), 3.91-3.61 (m, 1H), 2.68 (ddd, J = 14.5, 11.8, 1.6 Hz, 1H), 2.19- 1.94 (m, 1H), 1.88-1.73 (m, 2H), 1.73- 1.12 (m, 14H). | Starting with step 2 using tert-butyl 2- (hydroxymethyl) azepane-1- carboxylate |
| INT 2-5.1 | 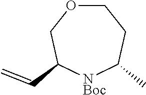 [SMILES: C=C[C@H]1COCC[C@H](C)N1C(=O)OC(C)(C)C] | δ 5.92 (ddd, J = 17.3, 10.7, 4.8 Hz, 1H), 5.20 (d, J = 11.5 Hz, 2H), 4.65 (s, 1H), 4.44-4.17 (m, 1H), 4.07-3.53 (m, 4H), 2.04 (m, 1H), 1.91-1.79 (m, 1H), 1.49 (s, 9H), 1.35 (d, J = 6.8 Hz, 3H). | Step 1 was omitted. tert-butyl (3S,5S)-3- (hydroxymethyl)-5- methyl-1,4- oxazepane-4- carboxylate (INT 1-8) in step 2. |

### Intermediates 2-6-2-9.

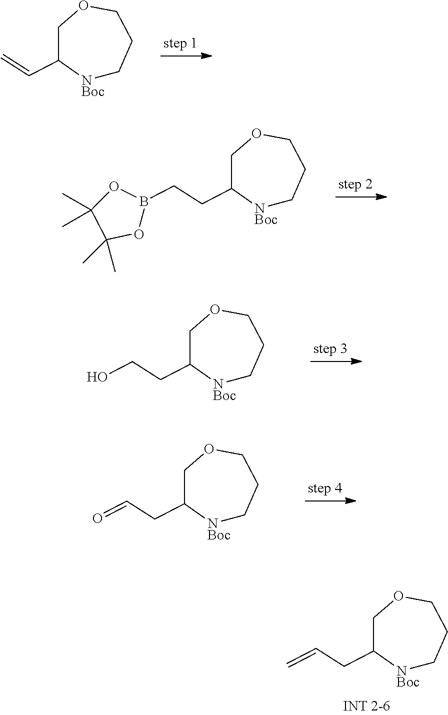 [SMILES: C=CC1COCCCN1C(=O)OC(C)(C)C ; CC(C)(C)OC(=O)N1CCCOCC1CCB1OC(C)(C)C(C)(C)O1 ; CC(C)(C)OC(=O)N1CCCOCC1CCO ; CC(C)(C)OC(=O)N1CCCOCC1CC=O ; C=CCC1COCCCN1C(=O)OC(C)(C)C]

Step 1: tert-Butyl 3-(2-(4,4,5,5-tetramethyl-1,3,2-dioxaborolan-2-yl) ethyl)-1,4-oxazepane-4-carboxylate. tert-Butyl 3-vinyl-1,4-oxazepane-4-carboxylate (1.9 mmol) was dissolved in 1,2-dichloroethane (10 mL) and the stirred solution was evacuated and refilled with argon (3×). To this solution chloro-1,5-cyclooctadiene iridium(I) dimer (0.29 mmol) was added, followed by 1,2-bis(diphenylphosphino)ethane (0.57 mmol), after which the resulting mixture was evacuated and refilled with argon again (3×). After stirring for 30 minutes at room temperature, the reaction mixture was cooled to 0° C. and a solution of pinacolborane (3.8 mmol) in dichloromethane (2 mL) was added dropwise over 15 minutes. After the addition, the ice bath was removed, and the mixture was stirred for an additional 2 hours at room temperature. The reaction mixture was then quenched with saturated aqueous ammonium chloride solution (5 mL) and the aqueous phase was extracted with dichloromethane (3×20 mL). The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 4.32-4.03 (m, 1H), 4.03-3.62 (m, 3H), 3.59-3.20 (m, 2H), 3.06-2.88 (m, 1H), 2.04-1.38 (m, 13H), 1.26 (s, 12H), 0.87-0.70 (m, 2H).

Step 2: tert-Butyl 3-(2-hydroxyethyl)-1,4-oxazepane-4-carboxylate. A premixed solution of sodium hydroxide (2 M aqueous)/hydrogen peroxide (30% aqueous) (2:1, 15 mL) was added dropwise to a solution of tert-butyl 3-(2-(4,4,5,5-tetramethyl-1,3,2-dioxaborolan-2-yl) ethyl)-1,4-oxazepane-4-carboxylate (1.9 mmol) in tetrahydrofuran (7.5 mL) at 0° C. After addition the reaction mixture was vigorously stirred at room temperature for 2 hours. The reaction was quenched with aqueous ammonium chloride solution (5 mL). The reaction mixture was extracted with ethyl acetate (2×15 mL). The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 4.47 (tdd, J=10.8, 5.8, 2.7 Hz, 1H), 4.09 (ddd, J=12.4, 7.2, 5.0 Hz, 2H), 3.89-3.76 (m, 1H), 3.77-3.57 (m, 1H), 3.57-3.45 (m, 1H), 3.45-3.35 (m, 1H), 3.27 (dd, J=13.4, 10.5 Hz, 1H), 2.94 (ddd, J=14.9, 11.7, 1.4 Hz, 1H), 1.94 (dtdd, J=14.5, 12.0, 5.6, 2.5 Hz, 1H), 1.73-1.43 (m, 11H), 1.35-1.19 (m, 1H).

Step 3: tert-Butyl 3-(2-oxoethyl)-1,4-oxazepane-4-carboxylate. To a solution of tert-butyl 3-(2-hydroxyethyl)-1,4-oxazepane-4-carboxylate (1.4 mmol) in dichloromethane (10 mL) at 0° C. was added Dess-Martin Periodinane (1.7 mmol). The mixture was stirred at room temperature for 3 hours. Saturated sodium bicarbonate solution (15 mL) was added to the mixture, the mixture was extracted with ethyl acetate (2×15 mL), the combined organic layers were dried over anhydrous sodium sulfate, and the filtrate was concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 9.77 (d, J=2.4 Hz, 1H), 4.90-4.53 (m, 1H), 4.08-3.69 (m, 3H), 3.64-3.39 (m, 2H), 3.14 (ddd, J=15.1, 10.9, 2.5 Hz, 1H), 2.61-2.48 (m, 2H), 2.02-1.52 (m, 2H), 1.48 (s, 9H).

Step 4: tert-Butyl 3-allyl-1,4-oxazepane-4-carboxylate (INT 2-6). To a suspension of methyltriphenylphosphonium bromide (0.94 mmol) in tetrahydrofuran (3 mL) at room temperature was added a 1.0 M solution of potassium bis(trimethylsilyl)amide (0.90 mL) dropwise to afford a suspension. The mixture was stirred for 1 hour at room temperature and was cooled to −78° C. whereupon a solution of tert-butyl 3-(2-oxoethyl)-1,4-oxazepane-4-carboxylate (0.37 mmol) in tetrahydrofuran (1 mL) was added dropwise. The resulting mixture was allowed to gradually warm to room temperature and stirred for 3 hours. The mixture was quenched with methanol and stirred for 15 minutes. Saturated aqueous ammonium chloride solution (5 mL) was added, and the mixture was extracted with ethyl acetate (2×10 mL). The combined organic phase was washed with brine and concentrated under reduced pressure. The residue was purified by silica gel chromatography eluting with ethyl acetate/hexanes. Fractions containing the product were pooled and concentrated under reduced pressure to yield the title compound. ¹H NMR (400 MHz, Chloroform-d) δ 5.78 (tdd, J=14.4, 9.6, 7.3 Hz, 1H), 5.17-4.93 (m, 2H), 4.47-4.12 (m, 1H), 4.10-3.74 (m, 2H), 3.68-3.30 (m, 2H), 3.12-2.96 (m, 1H), 2.23 (dq, J=18.4, 7.4, 6.7 Hz, 2H), 1.91 (dddd, J=14.5, 9.1, 6.1, 3.4 Hz, 1H), 1.78-1.55 (m, 2H), 1.49 (s, 9H).

The following intermediates were made in a similar fashion to INT 2-6 and are shown below in Table 2B. To prepare the below intermediates, different reagents/starting materials were used than some of those described in the steps toward INT 2-6 and are noted in the last column of Table 2B—“Changes to Procedure: Different Reagents/Starting Materials”. A person of ordinary skill in the art will readily recognize which reagents/starting materials used for the synthesis of INT 2-6 were replaced with the different reagents/starting materials noted below.

TABLE 2B
Intermediates

| | | | Changes to |
|---|---|---|---|
| | | | Procedure: |
| | | | Different |
| Inter- | | | Reagents/Starting |
| mediate | Structure | NMR | Materials |
| 2-7 | 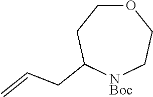 [SMILES: C=CCC1CCOCCN1C(=O)OC(C)(C)C] | ¹H NMR (400 MHz, Chloroform-d) δ 5.79 (dtt, J = 17.2, 10.0, 7.2 Hz, 1H), 5.14-4.98 (m, 2H), 4.26 (dq, J = 11.2, 6.9 Hz, 0.53H), 4.05 (dt, J = 11.6, 6.6 Hz, 0.44H), 4.00- 3.69 (m, 3H), 3.54-3.34 (m, 2H), 3.03 (dddd, J = 15.0, 9.8, 8.1, 1.5 Hz, 1H), 2.36- 2.08 (m, 3H), 1.80-1.62 (m, 1H), 1.49 (d, J = 6.3 Hz, 9H). | INT 2-3 in step 1 |
| 2-8 | 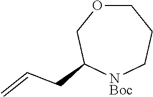 [SMILES: C=CC[C@H]1COCCCN1C(=O)OC(C)(C)C] | ¹H NMR (400 MHz, Chloroform-d) δ 5.94- 5.65 (m, 1H), 5.14-4.95 (m, 2H), 4.45- 3.79 (m, 3H), 3.67-3.29 (m, 2H), 3.03 (t, J = 12.8 Hz, 1H), 2.23 (dh, J = 20.3, 6.9 Hz, 2H), 1.49 (s, 12H). | INT 2-1 in step 1 |
| 2-9 | 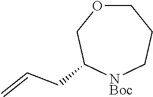 [SMILES: C=CC[C@@H]1COCCCN1C(=O)OC(C)(C)C] | ¹H NMR (400 MHz, Chloroform-d) δ 5.93- 5.61 (m, 1H), 5.16-4.92 (m, 2H), 4.43- 4.30 (m, 0.42H), 4.26-4.10 (m, 0.66H), 4.11-3.73 (m, 2H), 3.63-3.20 (m, 2H), 3.11-2.96 (m, 1H), 2.35-2.12 (m, 2H), 2.04-1.78 (m, 1H), 1.78-1.54 (m, 2H), 1.49 (s, 9H). | INT 2-4 in step 1 |

### Intermediates 3-1-3-2.

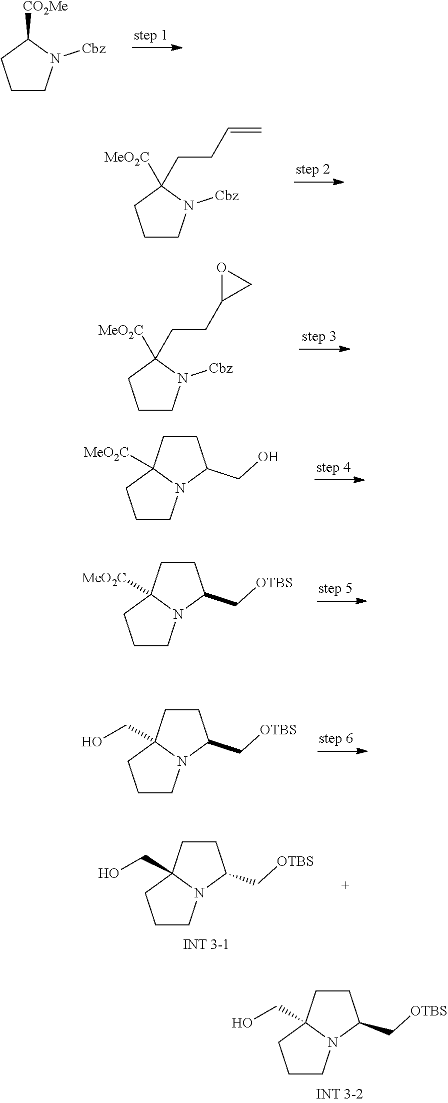

Step 1: 1-Benzyl 2-methyl 2-(but-3-en-1-yl)pyrrolidine-1,2-dicarboxylate. 1-Benzyl 2-methyl (S)-pyrrolidine-1,2-dicarboxylate (3.8 mol) in tetrahydrofuran (2 L) was added dropwise to a solution of lithium bis(trimethylsilyl)amide (5.7 mol) in tetrahydrofuran (5.7 L) at −60° C. 4-bromo-1-butene was added to the mixture. Upon completion, the reaction was partitioned between NH₄Cl_{(aq)} and ethyl acetate. The organic fraction was washed with brine, then dried over sodium sulfate and concentrated under reduced pressure. The resulting residue was purified by silica gel chromatography eluting with petroleum ether/ethyl acetate. Fractions containing the product were pooled and concentrated under reduced pressure to afford the title compound.

Step 2: 1-Benzyl 2-methyl 2-(2-(oxiran-2-yl)ethyl)pyrrolidine-1,2-dicarboxylate. To a stirred solution of 1-benzyl 2-methyl 2-(but-3-en-1-yl)pyrrolidine-1,2-dicarboxylate (0.94 mol) in dichloromethane (1.8 L) was added 3-chloroperbenzoic acid (1.13 mol) portionwise while maintaining the temperature between 20-30° C. Upon completion, the reaction was quenched by the dropwise addition of 10% sodium bisulfite solution in water (9 L) over 1h then extracted with dichloromethane (9 L). The organic fraction was washed with brine, dried over sodium sulfate, and concentrated under reduced pressure. The resulting residue was purified by silica gel chromatography eluting with petroleum ether/ethyl acetate. Fractions containing the product were pooled and concentrated under reduced pressure to afford the title compound. ¹H NMR (Chloroform-d, 400 MHz) δ 7.38-7.29 (m, 5H), 5.19-5.06 (m, 2H), 3.86-3.67 (m, 3H), 3.56-3.43 (m, 2H), 2.99-2.64 (m, 2H), 2.56-2.20 (m, 3H), 2.19-1.77 (m, 7H), 1.68-1.39 (m, 3H).

Step 3: Methyl 3-(hydroxymethyl)tetrahydro-1H-pyrrolizine-7a(5H)-carboxylate. To a mixture of 1-benzyl 2-methyl 2-(2-(oxiran-2-yl)ethyl)pyrrolidine-1,2-dicarboxylate in methanol (3 L) was added palladium on carbon (50.5 g of 10 wt. %) under a nitrogen atmosphere. The mixture was pressurized to 50 psi under a hydrogen atmosphere. Upon completion, the mixture was filtered and the filtrate was concentrated under reduced pressure to afford the title compound, which was used without further purification.

Step 4: rel-Methyl (3S,7aS)-3-(((tert-butyldimethylsilyl)oxy)methyl)tetrahydro-1H-pyrrolizine-7a(5H)-carboxylate. To a solution of methyl 3-(hydroxymethyl)tetrahydro-1H-pyrrolizine-7a(5H)-carboxylate (2.5 mol) and imidazole (2.8 mol) in dichloromethane (3 L) was added tert-butyldimethylsilyl chloride (2.5 mol). Upon completion, the reaction was diluted with water and extracted twice with dichloromethane (2×1 L). The combined organics were washed with brine, dried over sodium sulfate, filtered and concentrated under reduced pressure. The resulting residue was purified by silica gel chromatography eluting with petroleum ether/EtOAc. Fractions containing the product were pooled and concentrated under reduced pressure to afford the title compound. ¹H NMR (Chloroform-d, 400 MHz) δ 3.90 (dd, J=10.4, 4.4 Hz, 1H) 3.79 (br dd, J=10.4, 6.0 Hz, 1H) 3.72 (s, 3H) 3.37-3.27 (m, 1H) 3.01-2.92 (m, 1H) 2.84 (q, J=8.4 Hz, 1H) 2.46 (dt, J=12.4, 4.8 Hz, 1H) 2.18 (dt, J=12.4, 7.6 Hz, 1H) 1.96-1.68 (m, 7H) 1.66-1.55 (m, 1H) 0.90 (s, 9H) 0.06 (s, 6H).

Step 5: rel-((3S,7aS)-3-(((tert-butyldimethylsilyl)oxy)methyl)tetrahydro-1H-pyrrolizin-7a(5H)-yl)methanol. To a stirred mixture of lithium aluminum hydride (688 mmol) in tetrahydrofuran (1.1 L) at −30° C. was added methyl (3S,7aS)-3-(((tert-butyldimethylsilyl)oxy)methyl)tetrahydro-1H-pyrrolizine-7a(5H)-carboxylate (574 mmol) dropwise under a nitrogen atmosphere maintaining the temperature between −30 and −20° C. Upon completion, the reaction was quenched with water at −20° C., then gradually warmed to −10° C. The mixture was then filtered and the filter cake was rinsed with ethyl acetate (3×200 mL). The resulting filtrate was concentrated under reduced pressure, and the residue purified by silica gel chromatography eluting with petroleum ether/ethyl acetate. Fractions containing the product were pooled and concentrated under reduced pressure to afford the title compound. ¹H NMR (Chloroform-d, 400 MHz) δ 3.87 (dd, J=10.4, 6.0 Hz, 1H) 3.72 (dd, J=10.4, 6.0 Hz, 1H) 3.34-3.23 (m, 2H) 3.18-3.07 (m, 1H) 3.04 (br d, J=6.4 Hz, 1H) 2.90-2.81 (m, 1H) 2.75 (td, J=9.6, 6.4 Hz, 1H) 2.01-1.93 (m, 1H) 1.49-1.85 (m, 8H) 0.90 (s, 9H) 0.07 (s, 6H).

Step 6: ((3R,7aR)-3-(((tert-butyldimethylsilyl)oxy)methyl)tetrahydro-1H-pyrrolizin-7a(5H)-yl)methanol (INT 3-1). Rel-((3S,7aS)-3-(((tert-butyldimethylsilyl)oxy)methyl)tetrahydro-1H-pyrrolizin-7a(5H)-yl)methanol (455 mmol) was purified by chiral SFC. Column: (s,s) WHELK-01 (250 mm×50 mm, 10 um); mobile phase: [0.1% NH₃H₂O/IPA]; B %=40; 3.6 minutes. Early-eluting isomer. The title compound was obtained after concentration of product-containing fractions under reduced pressure. ¹H NMR (400 MHz, DMSO-d): 6 4.36 (br s, 1H) 3.78 (dd, J=10.4, 5.6 Hz, 1H) 3.64 (dd, J=10.4, 6.4 Hz, 1H) 3.16-3.07 (m, 1H) 3.07-2.91 (m, 2H) 2.70-2.62 (m, 2H) 1.90 (ddd, J=12.4, 7.2, 2.0 Hz, 1H) 1.72-1.43 (m, 7H) 1.30 (td, J=11.2, 7.6 Hz, 1H) 0.87 (s, 9H) 0.04 (s, 6H).

((3S,7aS)-3-(((tert-butyldimethylsilyl)oxy)methyl)tetrahydro-1H-pyrrolizin-7a(5H)-yl)methanol (INT 3-2). The title compound was obtained from the purification of Rel-((3S,7aS)-3-(((tert-butyldimethylsilyl)oxy)methyl)tetrahydro-1H-pyrrolizin-7a(5H)-yl)methanol (455 mmol) as the late eluting isomer using the conditions described above for INT 3-1. ¹H NMR (400 MHz, DMSO-d): δ 4.36 (br s, 1H) 3.78 (dd, J=10.4, 5.6 Hz, 1H) 3.64 (dd, J=10.4, 6.4 Hz, 1H) 3.15-3.08 (m, 1H) 3.07-2.93 (m, 2H) 2.69-2.63 (m, 2H) 1.90 (ddd, J=12.4, 7.2, 2.0 Hz, 1H) 1.70-1.43 (m, 7H) 1.36-1.25 (m, 1H) 0.87 (s, 10H) 0.04 (s, 6H).

### Intermediate 4-1.

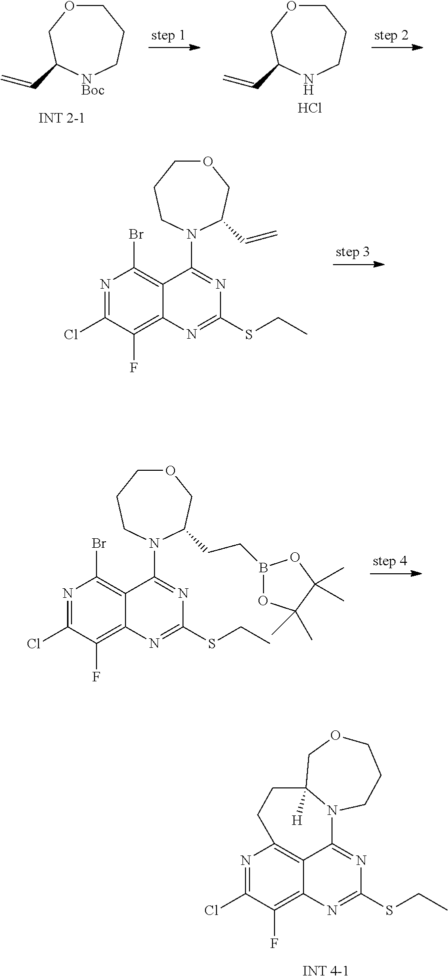

Step 1: (S)-3-Vinyl-1,4-oxazepane hydrogen chloride. To a stirred solution of INT 2-1 (1.9 mmol) in dichloromethane (3 ml) was added 4 M hydrochloric acid in 1, 4-dioxane (2.5 ml). Upon completion of the reaction, the mixture was concentrated under reduced pressure and the crude was taken forward without further purification. LCMS: 128.0.

Step 2: ((S)-4-(5-Bromo-7-chloro-2-(ethylthio)-8-fluoropyrido[4,3-d]pyrimidin-4-yl)-3-vinyl-1,4-oxazepane. To a stirred mixture of 5-bromo-4,7-dichloro-2-(ethylthio)-8-fluoropyrido[4,3-d]pyrimidine (2.5 mmol) and (S)-3-vinyl-1,4-oxazepane hydrogen chloride (1.9 mmol) in dichloromethane (6 ml) at 0° C. was slowly added N,N-diisopropylethylamine (1.5 ml). The mixture was then allowed to stir at room temperature. Upon completion, the mixture was partitioned between water and ethyl acetate, and the organic layer was dried over sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 447.0.

Step 3: (S)-4-(5-Bromo-7-chloro-2-(ethylthio)-8-fluoroquinazolin-4-yl)-3-(2-(4,4,5,5-tetramethyl-1,3,2-dioxaborolan-2-yl) ethyl)-1,4-oxazepane. (S)-4-(5-bromo-7-chloro-2-(ethylthio)-8-fluoropyrido[4,3-d]pyrimidin-4-yl)-3-vinyl-1,4-oxazepane (1.9 mmol) was dissolved in 1,2-dichloroethane (10 ml) and the stirred solution was evacuated and refilled with argon (3×). To this solution chloro-1,5-cyclooctadiene iridium(I) dimer (0.28 mmol) was added, followed by 1,2-bis(diphenylphosphino)ethane (0.56 mmol), after which the resulting mixture was evacuated and refilled with argon again (3×). After stirring for 30 minutes at room temperature, the reaction mixture was cooled to 0° C. and a solution of pinacolborane (3.8 mmol) in dichloromethane (2 ml) was added dropwise over 15 minutes. After the addition, the ice bath was removed, and the mixture was stirred for an additional 2 hours at room temperature while monitored by LCMS. The reaction mixture was then quenched with saturated aqueous ammonium chloride solution (5 mL) and the aqueous phase was extracted with dichloromethane (3×20 mL). The combined organic layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 574.3.

Step 4: (S)-2-Chloro-12-(ethylthio)-1-fluoro-4,5,5a,6,9,10-hexahydro-8H-7-oxa-3,10a,11,13-tetraazanaphtho[1,8-ab]heptalene (INT 4-1). (S)-4-(5-bromo-7-chloro-2-(ethylthio)-8-fluoroquinazolin-4-yl)-3-(2-(4,4,5,5-tetramethyl-1,3,2-dioxaborolan-2-yl) ethyl)-1,4-oxazepane (1.0 mmol) was dissolved in 1,4-dioxane (6 ml) and water (1.2 ml). Sodium carbonate (4.0 mmol) and (1,1′-bis(diphenylphosphino)ferrocene)-dichloropalladium (II) (0.15 mmol) were added and the mixture was degassed and heated at 140° C. After 15 minutes the reaction was complete. The mixture was cooled to room temperature, diluted with ethyl acetate (10 ml) and washed with aqueous ammonium chloride (10 ml). The organic layer was concentrated under reduced pressure. The residue was purified by silica gel chromatography eluting with ethyl acetate/hexanes. Fractions containing the product were pooled and concentrated under reduced pressure to yield the title compound. LCMS: 369.2.

### Intermediate 4-2.

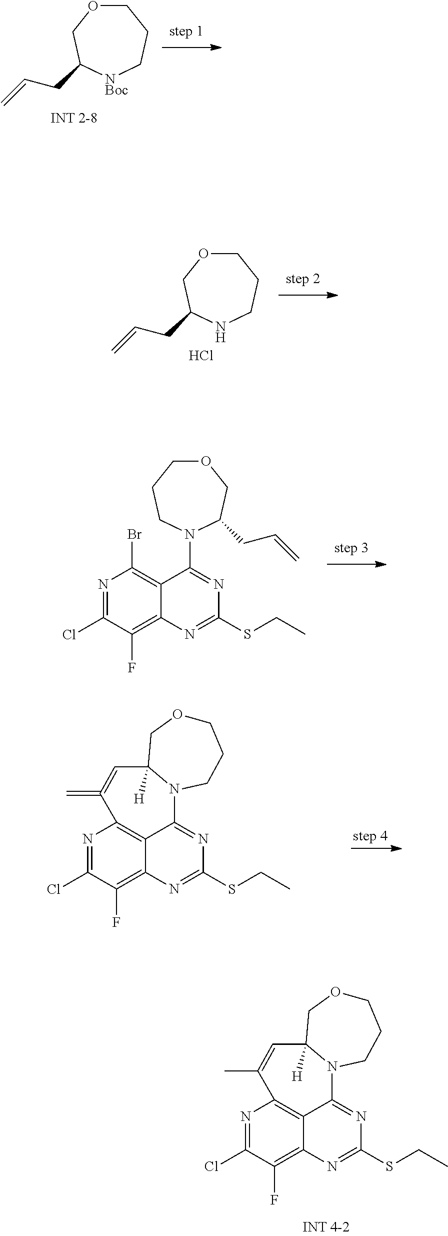

Step 1: (S)-3-Allyl-1,4-oxazepane hydrogen chloride. To a stirred solution of tert-butyl (S)-3-allyl-1,4-oxazepane-4-carboxylate (INT 2-8) (7.5 mmol) in dichloromethane (20 ml) was added 4 M HCl in 1, 4-dioxane (5 ml). Upon completion of the reaction, the mixture was concentrated under reduced pressure and the crude was taken forward without further purification. LCMS: 142.1.

Step 2: (S)-3-Allyl-4-(5-bromo-7-chloro-2-(ethylthio)-8-fluoropyrido[4,3-d]pyrimidin-4-yl)-1,4-oxazepane. To a stirred mixture of 5-bromo-4,7-dichloro-2-(ethylthio)-8-fluoropyrido[4,3-d]pyrimidine (7.3 mmol) and (S)-3-allyl-1,4-oxazepane hydrogen chloride (7.3 mmol) in dichloromethane (15 ml) at 0° C. was slowly added N,N-diisopropylethylamine (5.7 ml). The mixture was then allowed to stir at room temperature. Upon completion, the mixture was partitioned between water and ethyl acetate, and the organic layer was dried over sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 461.2.

Step 3: (S)-2-Chloro-12-(ethylthio)-1-fluoro-4-methylene-4,5,5a,6,9,10-hexahydro-8H-7-oxa-3,10a,11,13-tetraazanaphtho[1,8-ab]heptalene. (S)-3-allyl-4-(5-bromo-7-chloro-2-(ethylthio)-8-fluoropyrido[4,3-d]pyrimidin-4-yl)-1,4-oxazepane (1.5 mmol) was dissolved in N,N-dimethylformamide (5 ml), and tetrabutylammonium bromide (3.0 mmol), bis(tri-tert-butylphosphine)palladium(0) (0.3 mmol) and triethylamine (0.47 ml) were sequentially added. The mixture was purged with argon and heated at 80° C. LCMS showed complete conversion after 45 min and the reaction was cooled to room temperature. Ethyl acetate and water were added to the mixture. The organic layer was washed with brine and concentrated under reduced pressure. The residue was purified by silica gel chromatography eluting with ethyl acetate/hexanes. Fractions containing the product were pooled and concentrated under reduced pressure to yield the title compound. LCMS: 381.3.

Step 4: (S)-2-Chloro-12-(ethylthio)-1-fluoro-4-methyl-5a,6,9,10-tetrahydro-8H-7-oxa-3,10a,11,13-tetraazanaphtho[1,8-ab]heptalene (INT 4-2). To the solution of (S)-2-chloro-12-(ethylthio)-1-fluoro-4-methylene-4,5,5a,6,9,10-hexahydro-8H-7-oxa-3,10a,11,13-tetraazanaphtho[1,8-ab]heptalene (1.1 mmol) in toluene (5 ml) was added bis(tri-tert-butylphosphine)palladium (0.1 mmol) and isobutyryl chloride (0.1 mmol). The mixture was purged with argon and heated at 80° C. LCMS showed complete clean conversion after 1 hour. The mixture was cooled to room temperature. Ethyl acetate and water were added to the mixture. The organic layer was washed with brine and concentrated under reduced pressure. The residue was purified by silica gel chromatography eluting with ethyl acetate/hexanes. Fractions containing the product were pooled and concentrated under reduced pressure to yield the title compound. LCMS: 381.3.

### Intermediates 4-3A-4-3B.

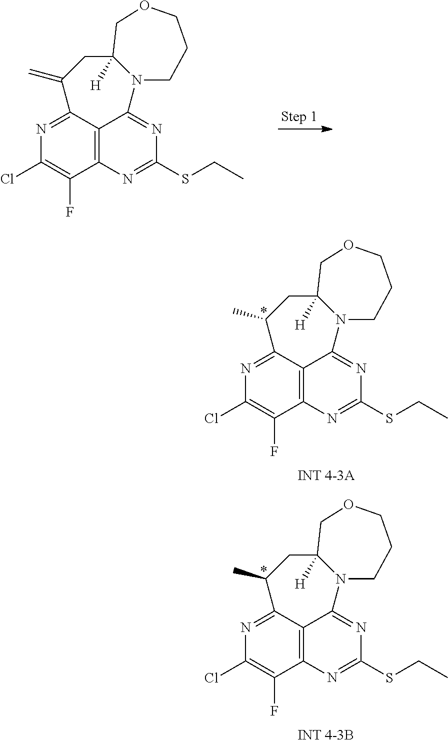

Step 1: (4R*,5aS)-2-chloro-12-(ethylthio)-1-fluoro-4-methyl-4,5,5a,6,9,10-hexahydro-8H-7-oxa-3,10a,11,13-tetraazanaphtho[1,8-ab]heptalene (INT 4-3A) and (4S*,5aS)-2-chloro-12-(ethylthio)-1-fluoro-4-methyl-4,5,5a,6,9,10-hexahydro-8H-7-oxa-3,10a,11,13-tetraazanaphtho[1,8-ab]heptalene (INT 4-3B). To a solution of (S)-2-chloro-12-(ethylthio)-1-fluoro-4-methylene-4,5,5a,6,9,10-hexahydro-8H-7-oxa-3,10a,11,13-tetraazanaphtho[1,8-ab]heptalene (1.2 mmol, prepared according to the procedure described for INT 4-2 step 3) in acetonitrile (8 mL) and water (4 mL) was added p-toluenesulfonyl hydrazide (12 mmol) and sodium acetate (19 mmol), and the mixture was heated at 80° C. for 1 h. After cooling to room temperature, the mixture was diluted with ethyl acetate and washed with water. The layers were separated, and the organic layer was washed with brine, dried over sodium sulfate, filtered over Celite, and concentrated under reduced pressure. The residue was purified by silica gel chromatography eluting with ethyl acetate/hexanes. Fractions containing the more polar product were pooled and concentrated to yield intermediate INT 4-3A (LCMS: 382.8), and fractions containing the less polar product were pooled and concentrated to yield intermediate INT 4-3B (LCMS: 382.9). The stereochemistry of the methyl group in INT 4-3A and INT 4-3B was arbitrarily assigned.

### Intermediate 4-4.

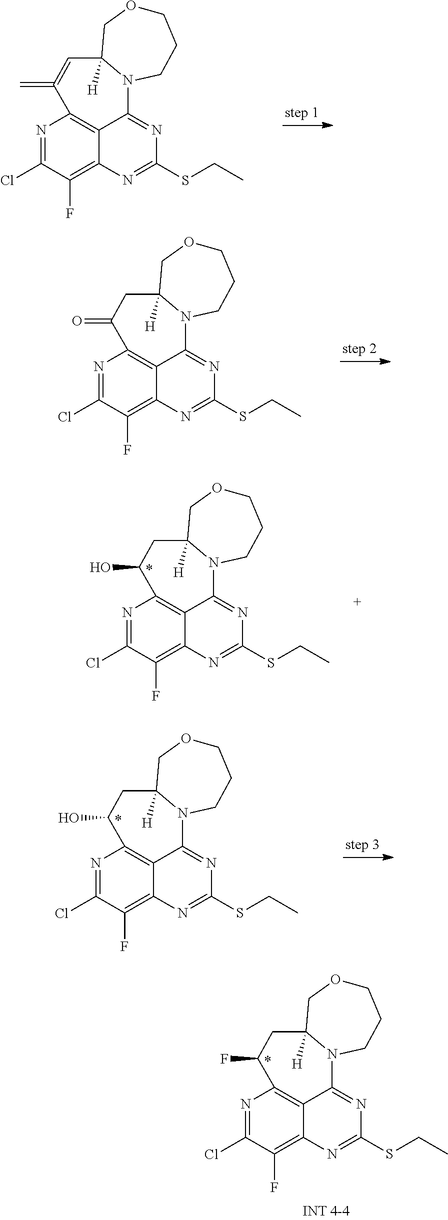

Step 1: (S)-2-chloro-12-(ethylthio)-1-fluoro-5a,6,9,10-tetrahydro-8H-7-oxa-3,10a,11,13-tetraazanaphtho[1,8-ab]heptalen-4(5H)-one. (S)-2-chloro-12-(ethylthio)-1-fluoro-4-methylene-4,5,5a,6,9,10-hexahydro-8H-7-oxa-3,10a,11,13-tetraazanaphtho[1,8-ab]heptalene (7.9 mmol, prepared according to the procedure described for INT 4-2 step 3) was taken up in dichloromethane (250 mL) and the mixture cooled to −78° C. Ozone gas was then passed through the solution for 20 min. Following reaction completion by LC/MS analysis, saturated aqueous sodium thiosulfate (200 mL) was added and the biphasic mixture warmed slowly to room temperature. The organic layer was collected, dried over magnesium sulfate, filtered, and concentrated under reduced pressure. The residue was purified by silica gel chromatography (eluent: ethyl acetate/hexanes gradient). Fractions containing the product were pooled and concentrated under reduced pressure to yield the title compound. LCMS: 382.9.

Step 2: (4R*,5aS)-2-chloro-12-(ethylthio)-1-fluoro-4,5,5a,6,9,10-hexahydro-8H-7-oxa-3,10a,11,13-tetraazanaphtho[1,8-ab]heptalen-4-ol. To a stirring solution of (R)-1-methyl-3,3-diphenylhexahydropyrrolo[1,2-c][1,3,2]oxazaborole (4.4 mmol, 1.0M in THF) at 0° C. under argon was added borane-THF (8.9 mmol, 1.0M in THF). The resulting mixture was stirred at 0° C. for 30 min and then a solution of (S)-2-chloro-12-(ethylthio)-1-fluoro-5a,6,9,10-tetrahydro-8H-7-oxa-3,10a,11,13-tetraazanaphtho[1,8-ab]heptalen-4(5H)-one (4.4 mmol) in THF (45 mL) was added and the reaction continued for 1 hour. Upon completion, the reaction was quenched by addition of methanol (10 mL) and allowed to warm to room temperature. The mixture was concentrated under reduced pressure and purified directly by silica gel chromatography (eluent: ethyl acetate/hexanes gradient). Fractions containing the product were collected and concentrated under reduced pressure to yield the title compound. The stereochemistry of the hydroxyl group was arbitrarily assigned. LCMS: 384.9.

Step 3: (4S*,5aS)-2-chloro-12-(ethylthio)-1,4-difluoro-4,5,5a,6,9,10-hexahydro-8H-7-oxa-3,10a,11,13-tetraazanaphtho[1,8-ab]heptalene (INT 4-4). To a stirring solution of (4R*,5aS)-2-chloro-12-(ethylthio)-1-fluoro-4,5,5a,6,9,10-hexahydro-8H-7-oxa-3,10a,11,13-tetraazanaphtho[1,8-ab]heptalen-4-ol (2.6 mmol, second eluting peak in step 2) in dichloromethane (25 mL) in a Teflon vial at −78° C. was added diethylaminosulfur trifluoride (3.1 mmol) slowly dropwise. The mixture was stirred at 78° C. for 1 hour. Upon completion, the mixture was warmed to 0° C. and water (10 mL) and saturated aqueous NaHCO₃ (10 mL) were added with vigorous stirring. The organic layer was collected and washed with brine, dried over magnesium sulfate, filtered, and concentrated under reduced pressure. The residue was purified by silica gel chromatography (eluent: ethyl acetate/hexanes gradient). Fractions containing the product were combined and concentrated under reduced pressure to yield the title compound. LCMS: 386.9.

### Intermediate 4-5.

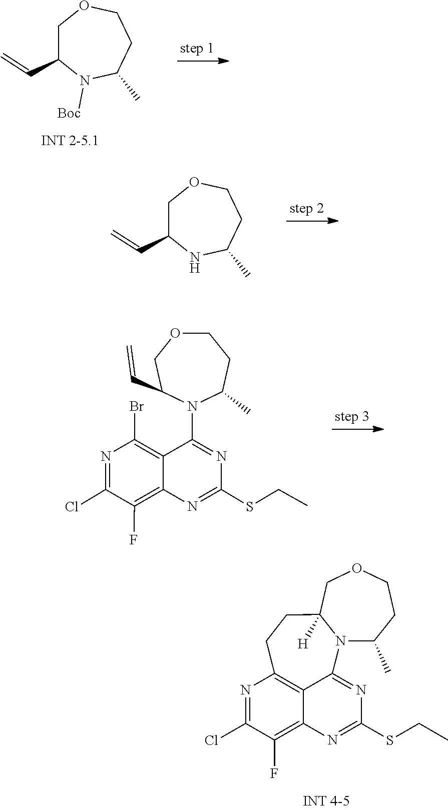

Step 1: (3S,5S)-5-methyl-3-vinyl-1,4-oxazepane hydrochloride. To a mixture of tert-butyl (3S,5S)-3-(hydroxymethyl)-5-methyl-1,4-oxazepane-4-carboxylate (INT 2-5.1) (0.46 mmol) in dichloromethane (2 mL) was added 4M hydrogen chloride in dioxane (4.6 mmol), and the reaction was stirred at room temperature for 90 minutes. It was concentrated in vacuo to give the title compound, which was directly used for the next step without further purification. ¹H NMR (400 MHz, MeOD) δ 5.98 (ddd, J=17.2, 10.5, 7.9 Hz, 1H), 5.62-5.47 (m, 2H), 4.15 (td, J=8.1, 4.1 Hz, 1H), 4.07-3.94 (m, 2H), 3.92-3.82 (m, 1H), 3.80-3.70 (m, 2H), 2.19-2.01 (m, 2H), 1.45 (d, J=6.8 Hz, 3H).

Step 2: (3S,5S)-4-(5-bromo-7-chloro-2-(ethylthio)-8-fluoropyrido[4,3-d]pyrimidin-4-yl)-5-methyl-3-vinyl-1,4-oxazepane. To a stirred mixture of 5-bromo-4,7-dichloro-2-(ethylthio)-8-fluoropyrido[4,3-d]pyrimidine (0.53 mmol) and (3S,5S)-5-methyl-3-vinyl-1,4-oxazepane hydrochloride (0.46 mmol) in dichloromethane (1 ml) at 0° C. was slowly added N,N-diisopropylethylamine (0.36 ml). The mixture was then allowed to stir at room temperature. Upon completion, the mixture was partitioned between water and ethyl acetate, and the organic layer was dried over sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 461.0.

Step 3: (5aS,1OS)-2-chloro-12-(ethylthio)-1-fluoro-10-methyl-4,5,5a,6,9,10-hexahydro-8H-7-oxa-3,10a,11,13-tetraazanaphtho[1,8-ab]heptalene (INT 4-5). To a solution of (3S,5S)-4-(5-bromo-7-chloro-2-(ethylthio)-8-fluoropyrido[4,3-d]pyrimidin-4-yl)-5-methyl-3-vinyl-1,4-oxazepane (1.2 mmol) in tetrahydrofuran (1.2 mL) was added 0.5 M 9-borabicyclo(3.3.1)nonane in tetrahydrofuran (3.4 mL), and the reaction was allowed to stir at room temperature for 16 hours. To this, 2M aqueous sodium carbonate (2.5 mL) and dioxane (11 mL) were added, and the mixture was degassed with argon. To this, (1,1′-bis(diphenylphosphino)ferrocene)-dichloropalladium(II) (0.18 mmol) was added, and the reaction was sealed and heated to 100° C. for 1 hour. It was cooled to ambient temperature, partitioned between water and ethyl acetate, and the aqueous layer was extracted three times with ethyl acetate. The combined organics layers were washed with brine, dried over anhydrous sodium sulfate, filtered, and concentrated under reduced pressure. The residue was purified by silica gel chromatography eluting with ethyl acetate/hexanes. Fractions containing the product were pooled and concentrated under reduced pressure to yield the title compound. LCMS: 461.0.

### Intermediates 5-1-5-3.

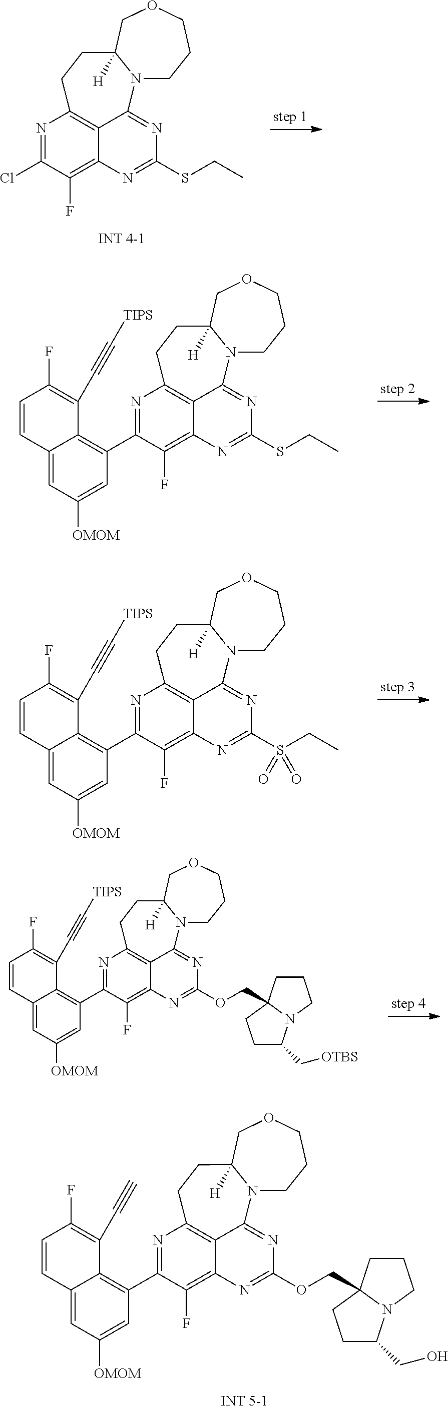

Step 1: (S)-12-(Ethylthio)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. To a solution of (S)-2-chloro-12-(ethylthio)-1-fluoro-4,5,5a,6,9,10-hexahydro-8H-7-oxa-3,10a,11,13-tetraazanaphtho[1,8-ab]heptalene (3.6 mmol) in tetrahydrofuran (55 mL) and water (11 mL) was added 2-[2-fluoro-6-(methoxymethoxy)-8-(4,4,5,5-tetramethyl-1,3,2-dioxaborolan-2-yl)-1-naphthyl]ethynyl-triisopropyl-silane (5.5 mmol), tripotassium phosphate (11.1 mmol) and mesylate[(di(1-adamantyl)-n-butylphosphine)-2-(2′-amino-1,1′-biphenyl)]palladium(II) (0.74 mmol). After the reaction mixture was evacuated and refilled with argon (3×), it was heated to 65° C. for 1 hour. The reaction mixture was cooled to room temperature, diluted with ethyl acetate, then washed with water and saturated aqueous ammonium chloride solution. Combined organic extracts were washed with brine, dried, filtered and concentrated. The crude product was purified by silica gel column chromatography (0% to 100% ethyl acetate in hexanes) to give the title compound. LCMS: 719.4.

Step 2: (S)-12-(Ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. To a stirred solution of (S)-12-(ethylthio)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.97 mmol) in dichloromethane (13 mL) at 0° C. was added 3-chloroperoxybenzoic acid (2.14 mmol) in one portion. After stirring for 30 minutes at room temperature, the reaction mixture was diluted with dichloromethane (30 mL) and washed with saturated aqueous solution of sodium bicarbonate (10 mL), dried and concentrated to afford the crude product, which was used without purification. LCMS: 751.4.

Step 3: (S)-12-(((3S,7aS)-3-(((tert-butyldimethylsilyl)oxy)methyl)tetrahydro-1H-pyrrolizin-7a(5H)-yl)methoxy)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. Lithium bis(trimethylsilyl)amide (1.0 M solution in tetrahydrofuran, 0.9 mL) was added dropwise over 5 minutes via syringe to a stirred solution of (S)-12-(ethylsulfonyl)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.4 mmol) and INT 3-2 (0.7 mmol) in 2-methyltetrahydrofuran (3.3 mL) at 0° C. under an argon atmosphere. After 10 minutes, the resulting mixture was allowed to warm to room temperature. The resulting mixture was stirred at room temperature for 30 minutes. The mixture was then diluted with water (20 mL) and extracted with ethyl acetate (3×25 mL). The organic layer was washed with brine (20 mL), dried over anhydrous sodium sulfate, filtered, and concentrated under reduced pressure to yield the title compound which was used without further purification. LCMS: 942.6.

Step 4: ((3S,7aS)-7a-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methanol (INT 5-1). Cesium fluoride (4.25 mmol) was added in one portion to a stirred solution of (S)-12-(((3S,7aS)-3-(((tert-butyldimethylsilyl)oxy)methyl)tetrahydro-1H-pyrrolizin-7a(5H)-yl)methoxy)-1-fluoro-2-(7-fluoro-3-(methoxymethoxy)-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (0.3 mmol) in N,N-dimethylformamide (2.7 mL) at room temperature. The resulting mixture was heated to 45° C. for 16 hours. The mixture was then filtered through Celite and concentrated under reduced pressure. The resulting residue was co-evaporated with toluene (2×5 mL). The residue was then purified by flash column chromatography on silica gel (0% to 30% 10% ammonium hydroxide in ethanol/dichloromethane). Fractions containing the product were pooled and concentrated under reduced pressure to yield the title compound. ¹H NMR (400 MHz, Methanol-d4) δ 8.03-7.90 (m, 1H), 7.66 (t, J=3.1 Hz, 1H), 7.44-7.30 (m, 2H), 5.41-5.31 (m, 2H), 4.53 (td, J=14.7, 6.2 Hz, 1H), 4.44-4.35 (m, 1H), 4.28-4.18 (m, 1H), 4.12-3.76 (m, 6H), 3.75-3.64 (m, 3H), 3.53-3.49 (m, 3H), 3.24-3.00 (m, 2H), 2.96-2.87 (m, 1H), 2.84-2.74 (m, 1H), 2.42-2.11 (m, 4H), 2.08-1.59 (m, 9H). LCMS: 672.4

The following intermediates were made in a similar fashion to INT 5-1 and are shown below in Table 2C. To prepare the below intermediates, different reagents/starting materials were used than previously described in the steps toward INT 5-1 and are noted in the last column of Table 2C—“Changes to Procedure: Different Reagents/Starting Materials”. A person of ordinary skill in the art will readily recognize which reagents/starting materials used for the synthesis of INT 5-1 were replaced with the different reagents/starting materials noted below.

TABLE 2C
Intermediates

| | | | Changes to Procedure: Different |
|---|---|---|---|
| Intermediate | Structure | LCMS | Reagents/Starting Materials |
| INT 5-2 | 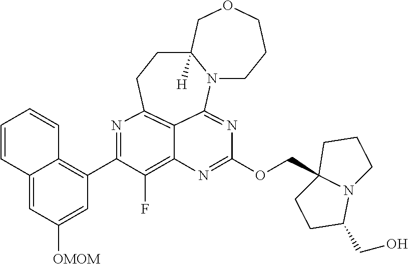 | 630.4 | 2-(3-(methoxymethoxy)naphthalen-1- yl)-4,4,5,5-tetramethyl-1,3,2- dioxaborolane in step 1. |
| INT 5-3 | 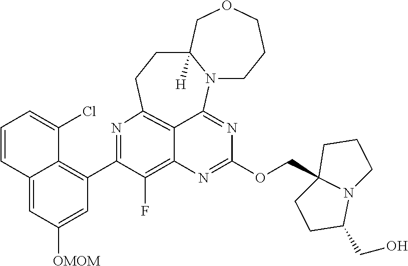 | 664.0 | 2-(8-chloro-3- (methoxymethoxy)naphthalen-1-yl)- 4,4,5,5-tetramethyl-1,3,2- dioxaborolane in step 1 |

### Intermediate 5-4.

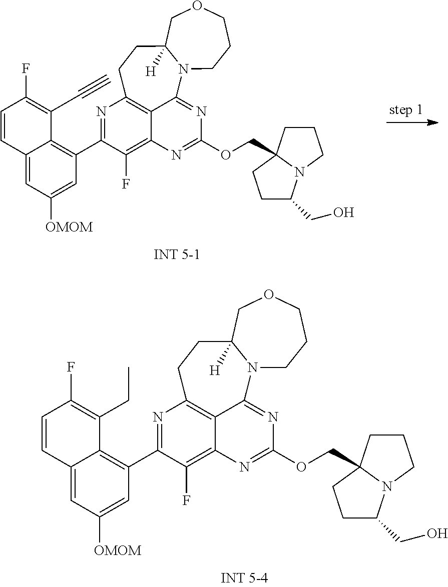

Step 1: ((3S,7aS)-7a-((((S)-2-(8-ethyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methanol (INT 5-4). A suspension of INT 5-1 (0.3 mmol) and palladium hydroxide on carbon (1.4 mmol, 20% wt Pd) in tetrahydrofuran (5 mL) was evacuated under vacuum and backfilled with hydrogen three times. The reaction mixture was vigorously stirred at room temperature for 12 hours under a hydrogen atmosphere. Upon completion, the solution was filtered through a short pad of Celite, washed with dichloromethane (3×10 mL), concentrated under reduced pressure and purified by column chromatography (0-30% ethanol with 10% ammonium hydroxide in dichloromethane) to afford the title compound. LCMS: 676.1.

### Intermediates 5-5-5-6.

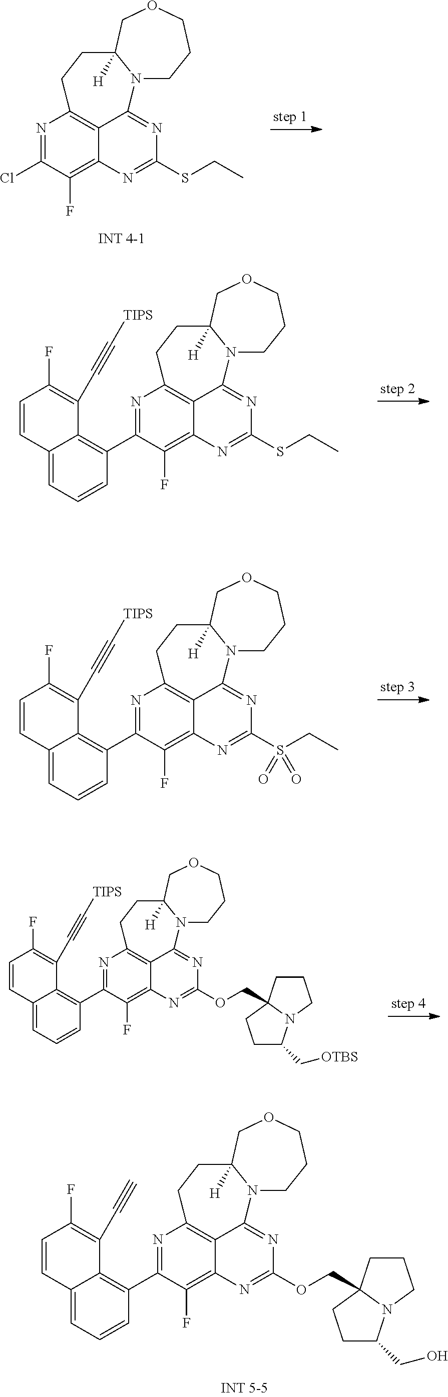

Step 1: (S)-12-(ethylthio)-1-fluoro-2-(7-fluoro-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. To a solution of INT 4-1 (3.0 mmol), potassium phosphate (9.0 mmol), 2-[2-fluoro-8-(4,4,5,5-tetramethyl-1,3,2-dioxaborolan-2-yl)-1-naphthyl]ethynyl-triisopropyl-silane (4.5 mmol) in tetrahydrofuran (30 mL) and degassed water (6 mL) was added [2-(2-aminophenyl)phenyl]-methylsulfonyloxy-palladium; bis(1-adamantyl)-butyl-phosphane (0.6 mmol) under nitrogen atmosphere. The resulting mixture was stirred at 70° C. for 90 minutes under an argon atmosphere. Upon completion, the mixture was diluted with ethyl acetate and the organic layer was washed with sat. aq. ammonium chloride solution, brine, dried over anhydrous sodium sulfate, filtered, and concentrated under reduced pressure. The residue was purified by flash column chromatography on silica gel (20 to 60% ethyl acetate in hexanes). Fractions containing the product were pooled and concentrated under reduced pressure to yield the title compound. LCMS: 659.4.

Step 2: (S)-12-(ethylsulfonyl)-1-fluoro-2-(7-fluoro-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. To a solution of (S)-12-(ethylthio)-1-fluoro-2-(7-fluoro-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (2.6 mmol) in 40 mL dichloromethane at 0° C. was added 3-chloroperbenzoic acid (5.7 mmol) in portions. The resulting mixture was stirred at room temperature for 2 hours. Upon completion the mixture was diluted with more dichloromethane and the organic layer washed with sat. aq. sodium bicarbonate, sat. aq. sodium thiosulfate, brine, dried over anhydrous sodium sulfate, filtered, and concentrated under reduced pressure to afford the title compound which was used directly in the next step without further purification. LCMS: 691.4.

Step 3: (S)-12-(((3S,7aS)-3-(((tert-butyldimethylsilyl)oxy)methyl)tetrahydro-1H-pyrrolizin-7a(5H)-yl)methoxy)-1-fluoro-2-(7-fluoro-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene. To a solution of (S)-12-(ethylsulfonyl)-1-fluoro-2-(7-fluoro-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene from Step 2 and INT 3-2 (2.9 mmol) in 30 mL 2-methyltetrahydrofuran was added dropwise a solution of lithium hexamethyldisilazide in tetrahydrofuran (1 M, 3.9 mL) at 0° C. and the mixture was stirred at room temperature for 1 hour. The mixture was then diluted with ethyl acetate and the organic layer washed with sat. aq. sodium bicarbonate, brine, dried over anhydrous sodium sulfate, filtered, and concentrated under reduced pressure. The residue was purified by flash column chromatography on silica gel (0 to 40% methanol in dichloromethane). Fractions containing the product were pooled and concentrated under reduced pressure to yield the title compound. LCMS: 882.5.

Step 4: ((3S,7aS)-7a-((((S)-2-(8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methanol (INT 5-5). To a solution of (S)-12-(((3S,7aS)-3-(((tert-butyldimethylsilyl)oxy)methyl)tetrahydro-1H-pyrrolizin-7a(5H)-yl)methoxy)-1-fluoro-2-(7-fluoro-8-((triisopropylsilyl)ethynyl)naphthalen-1-yl)-4,5,5a,6,9,10-hexahydro-8H-7-oxa-3,10a,11,13-tetraazanaphtho[1,8-ab]heptalene (1.8 mmol) in 20 mL tetrahydrofuran was added a solution of tetrabutylammonium fluoride in tetrahydrofuran (1 M, 5.4 mL) and the mixture was allowed to stir at room temperature for 18 hours. The mixture was concentrated under reduced pressure and the residue was purified by flash column chromatography on silica gel (0-30% 10% ammonium hydroxide in ethanol/dichloromethane). Fractions containing the product were pooled and concentrated under reduced pressure to yield the title compound. ¹H NMR (400 MHz, Methanol-d4) δ 8.17-8.07 (m, 2H), 7.74-7.59 (m, 2H), 7.45 (td, J=9.0, 1.9 Hz, 1H), 4.55 (td, J=14.2, 6.2 Hz, 1H), 4.42 (dd, J=10.4, 1.8 Hz, 1H), 4.25 (dd, J=10.5, 8.0 Hz, 1H), 4.11 (dd, J=13.9, 3.5 Hz, 1H), 4.06-3.78 (m, 5H), 3.78-3.67 (m, 2H), 3.25-2.92 (m, 3H), 2.90-2.77 (m, 1H), 2.45-2.15 (m, 4H), 2.08-1.62 (m, 10H). LCMS: 612.3.

The following intermediates were made in a similar fashion to INT 5-5 and are shown below in Table 2D. To prepare the below intermediates, different reagents/starting materials were used than previously described in the steps toward INT 5-5 and are noted in the last column of Table 2D—“Changes to Procedure: Different Reagents/Starting Materials”. A person of ordinary skill in the art will readily recognize which reagents/starting materials used for the synthesis of INT 5-5 were replaced with the different reagents/starting materials noted below.

TABLE 2D
Intermediates

| | | | Changes to Procedure: Different |
|---|---|---|---|
| Intermediate | Structure | LCMS | Reagents/Starting Materials |
| INT 5-6 | 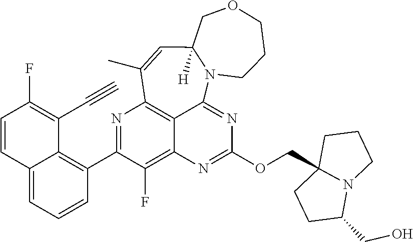 | 624.0 | INT 4-2 in step 1 |

### Intermediates 5-7-5-19.

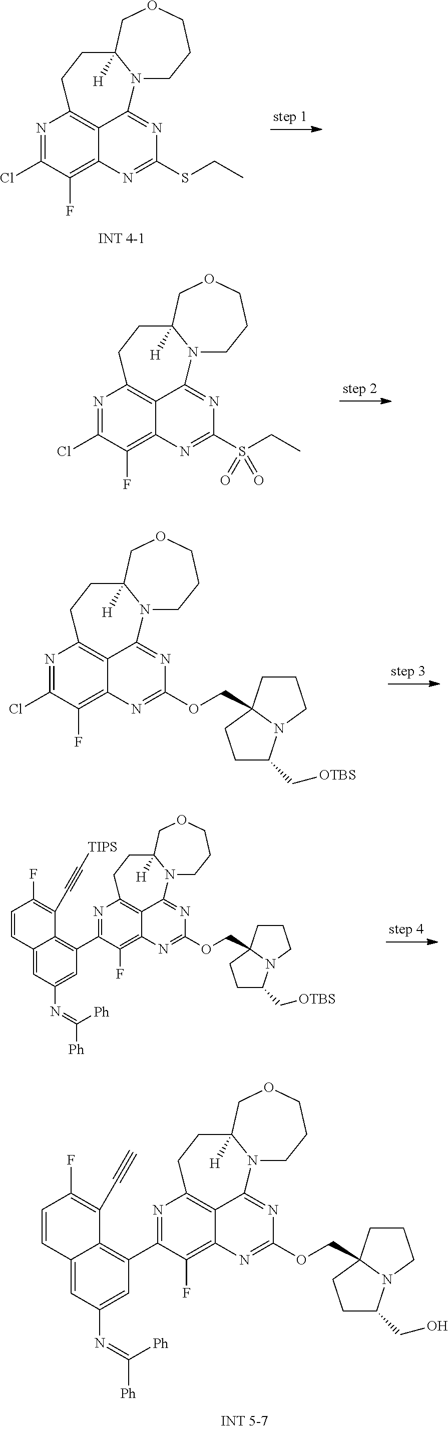

Step 1: (S)-2-chloro-12-(ethylsulfonyl)-1-fluoro-4,5,5a,6,9,10-hexahydro-8H-7-oxa-3,10a,11,13-tetraazanaphtho[1,8-ab]heptalene. To a solution of INT 4-1 (0.5 mmol) in 10 mL dichloromethane at 0° C. was added 3-chloroperbenzoic acid (1.2 mmol) in portions. The resulting mixture was stirred at room temperature for 2 hours. Upon completion the mixture was diluted with more dichloromethane and the organic layer washed with sat. aq. sodium bicarbonate, sat. aq. sodium thiosulfate, brine, dried over anhydrous sodium sulfate, filtered, and concentrated under reduced pressure to afford the title compound which was used directly in the next step without further purification. LCMS: 401.1.

Step 2: (S)-12-(((3S,7aS)-3-(((tert-butyldimethylsilyl)oxy)methyl)tetrahydro-1H-pyrrolizin-7a(5H)-yl)methoxy)-2-chloro-1-fluoro-4,5,5a,6,9,10-hexahydro-8H-7-oxa-3,10a,11,13-tetraazanaphtho[1,8-ab]heptalene. To a solution of (S)-2-chloro-12-(ethylsulfonyl)-1-fluoro-4,5,5a,6,9,10-hexahydro-8H-7-oxa-3,10a,11,13-tetraazanaphtho[1,8-ab]heptalene from step 1 and [(3S,8S)-3-[[tert-butyl(dimethyl)silyl]oxymethyl]-1,2,3,5,6,7-hexahydropyrrolizin-8-yl]methanol (0.7 mmol) in 10 mL 2-methyltetrahydrofuran was added dropwise a solution of lithium hexamethyldisilazide in tetrahydrofuran (1 M, 0.8 mL) at 0° C. and the mixture was stirred at room temperature for 1 hour. The mixture was then diluted with ethyl acetate and the organic layer washed with sat. aq. sodium bicarbonate, brine, dried over anhydrous sodium sulfate, filtered, and concentrated under reduced pressure. The residue was purified by flash column chromatography on silica gel (0 to 30% methanol in dichloromethane). Fractions containing the product were pooled and concentrated under reduced pressure to yield title compound. LCMS 592.4.

Step 3: N-(4-((S)-12-(((3S,7aS)-3-(((tert-butyldimethylsilyl)oxy)methyl)tetrahydro-1H-pyrrolizin-7a(5H)-yl)methoxy)-1-fluoro-4,5,5a,6,9,10-hexahydro-8H-7-oxa-3,10a,11,13-tetraazanaphtho[1,8-ab]heptalen-2-yl)-6-fluoro-5-((triisopropylsilyl)ethynyl)naphthalen-2-yl)-1,1-diphenylmethanimine. To a solution of (S)-12-(((3S,7aS)-3-(((tert-butyldimethylsilyl)oxy)methyl)tetrahydro-1H-pyrrolizin-7a(5H)-yl)methoxy)-2-chloro-1-fluoro-4,5,5a,6,9,10-hexahydro-8H-7-oxa-3,10a,11,13-tetraazanaphtho[1,8-ab]heptalene (1.4 mmol), potassium phosphate (5.4 mmol), N-[6-fluoro-4-(4,4,5,5-tetramethyl-1,3,2-dioxaborolan-2-yl)-5-(2-triisopropylsilylethynyl)-2-naphthyl]-1,1-diphenyl-methanimine (2.8 mmol) in tetrahydrofuran (13.5 mL) and degassed water (3.5 mL) was added [2-(2-aminophenyl)phenyl]-methylsulfonyloxy-palladium; bis(1-adamantyl)-butyl-phosphane (0.27 mmol) under a nitrogen atmosphere. The resulting mixture was stirred at 70° C. for 2 hours under an argon atmosphere. Upon completion, the mixture was diluted with ethyl acetate and the organic layer was washed with water, brine, dried over anhydrous sodium sulfate, filtered, and concentrated under reduced pressure. The residue was purified by flash column chromatography on silica gel (0-20% 10% ammonium hydroxide in ethanol/dichloromethane). Fractions containing the product were pooled and concentrated under reduced pressure to yield the title compound. LCMS: 1061.6.

Step 4: ((3S,7aS)-7a-((((S)-2-(3-((diphenylmethylene)amino)-8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methanol (INT 5-7). To a solution of N-(4-((S)-12-(((3S,7aS)-3-(((tert-butyldimethylsilyl)oxy)methyl)tetrahydro-1H-pyrrolizin-7a(5H)-yl)methoxy)-1-fluoro-4,5,5a,6,9,10-hexahydro-8H-7-oxa-3,10a,11,13-tetraazanaphtho[1,8-ab]heptalen-2-yl)-6-fluoro-5-((triisopropylsilyl)ethynyl)naphthalen-2-yl)-1,1-diphenylmethanimine (1.3 mmol) in N,N′-dimethylformamide was added cesium fluoride (27 mmol) and the mixture was stirred vigorously at 60° C. for 18 hours. Upon completion, the mixture was diluted with ethyl acetate and the organic layer was washed with water, brine, dried over anhydrous sodium sulfate, filtered, and concentrated under reduced pressure. The residue was purified by flash column chromatography on silica gel (0-30% 10% ammonium hydroxide in ethanol/dichloromethane). Fractions containing the product were pooled and concentrated under reduced pressure to yield the title compound. ¹H NMR (400 MHz, Methanol-d4) δ 7.85-7.79 (m, 1H), 7.78-7.74 (m, 2H), 7.57-7.51 (m, 1H), 7.46 (m, 2H), 7.35-7.27 (m, 5H), 7.25-7.17 (m, 3H), 4.53 (td, J=14.0, 6.2 Hz, 1H), 4.40 (d, J=10.4 Hz, 1H), 4.23 (dd, J=10.5, 8.7 Hz, 1H), 4.11-4.06 (m, 1H), 4.04-3.77 (m, 5H), 3.77-3.63 (m, 3H), 3.38-3.28 (in, TH-), 3.22-2.90 (m, 3H), 2.89-2.72 (in, 1H), 2.41-2.09 (m, 4H), 2.02-1.55 (in, 8H). LCMS: 791.4.

The following intermediates were made in a similar fashion to TNT 5-7 and are shown below in Table 2E. To prepare the below intermediates, different reagents/starting materials were used than previously described in the steps toward TNT 5-7 and are noted in the last column of Table 2E—“Changes to Procedure: Different Reagents/Starting Materials”. A person of ordinary skill in the art will readily recognize which reagents/starting materials used for the synthesis of INT 5-7 were replaced with the different reagents/starting materials noted below.

TABLE 2E
Intermediates

| Inter- | | | Changes to |
|---|---|---|---|
| me- | | | Procedure: Different |
| diate | Structure | LCMS | Reagents/Starting Materials |
| INT 5-8 | 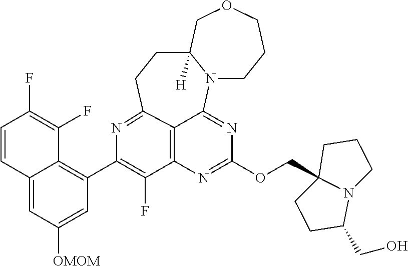 | 666.3 | 2-[7,8-difluoro-3- (methoxymethoxy)- 1-naphthyl]-4,4,5,5- tetramethyl-1,3,2- dioxaborolane in step 3. |
| INT 5-9 | 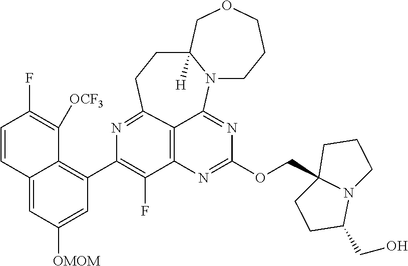 | 732.3 | 2-(7-fluoro-3- (methoxymethoxy)-8- (trifluoromethoxy) naphthalen-1-yl)- 4,4,5,5-tetramethyl-1,3,2- dioxaborolane in step 3. |
| INT 5-10 | 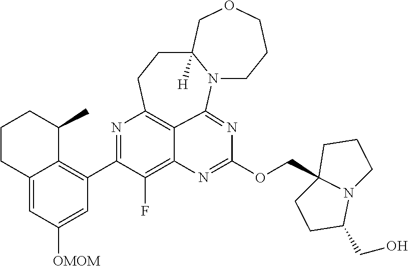 | 648.4 | INT 1-3 in step 3. |
| INT 5-11 | 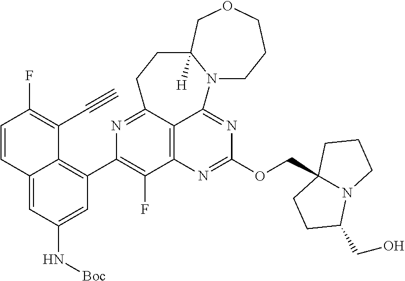 | 727.4 | tert-butyl N-[6- fluoro-4-(4,4,5,5- tetramethyl-1,3,2- dioxaborolan-2-yl)-5- (2-triisopropyl- silylethynyl)-2- naphthyl]carbamate in step 3. |
| INT 5-12 | 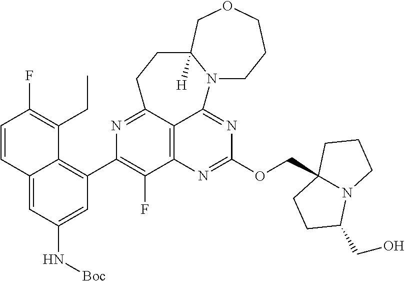 | 731.4 | Prepared according to the procedure described for INT 5-4 step 1 using INT 5-11. |
| INT 5-13 | 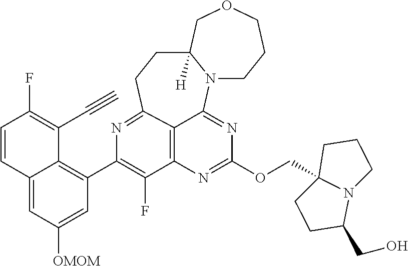 | 672.4 | INT 3-1 in step 2. 2-[2-fluoro-6- (methoxymethoxy)- 8-(4,4,5,5- tetramethyl-1,3,2- dioxaborolan-2-yl)-1- naphthyl]ethynyl- triisopropyl-silane in step 3. |
| INT 5-14 | 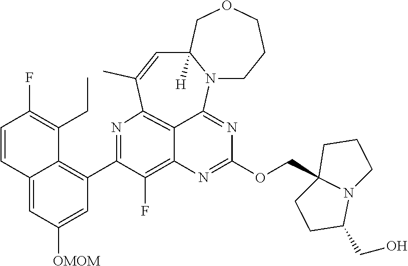 | 688.4 | INT 4-2 in step 1. 2-[8-ethyl-7-fluoro- 3-(methoxymethoxy)- 1-naphthyl]- 4,4,5,5-tetramethyl-1,3,2- dioxaborolane in step 3. |
| INT 5-15 | 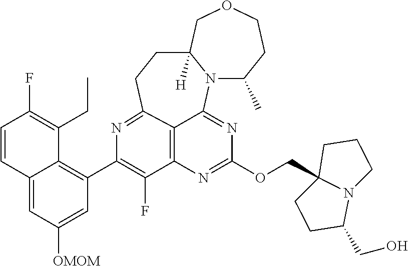 | 690.3 | INT 4-5 in step 1. 2-[8-ethyl-7-fluoro- 3-(methoxymethoxy)- 1-naphthyl]- 4,4,5,5-tetramethyl-1,3,2- dioxaborolane in step 3. |
| INT 5-16 | 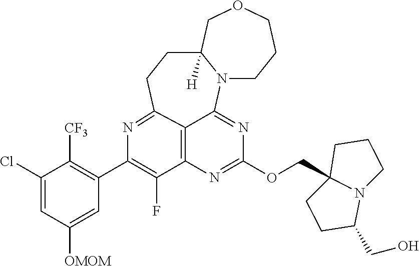 | 682.1 | 2-(3-chloro-5- (methoxymethoxy)-2- (trifluoromethyl) phenyl)-4,4,5,5- tetramethyl-1,3,2- dioxaborolane in step 3. |
| INT 5-17 | 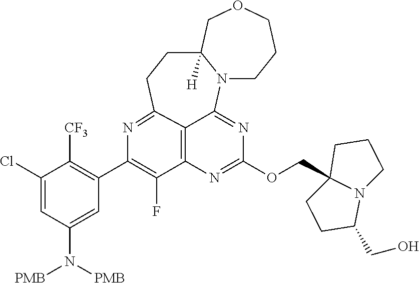 | 877.2 | INT 1-9 in step 3. |
| INT 5-18 | 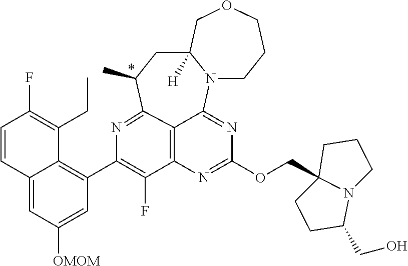 | 690.3 | INT 4-3B in step 1. 2-[8-ethyl-7-fluoro- 3-(methoxymethoxy)-1- naphthyl]- 4,4,5,5-tetramethyl-1,3,2- dioxaborolane in step 3. |
| INT 5-19 | 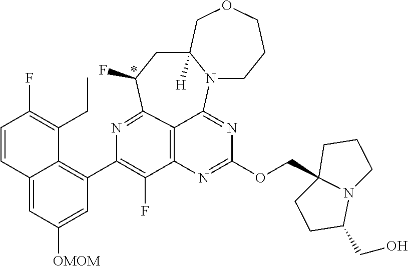 | 694.4 | INT 4-4 in step 1. 2-[8-ethyl-7-fluoro- 3-(methoxymethoxy)- 1-naphthyl]- 4,4,5,5-tetramethyl-1,3,2- dioxaborolane in step 3. |

### Intermediates 6-1-6-5.

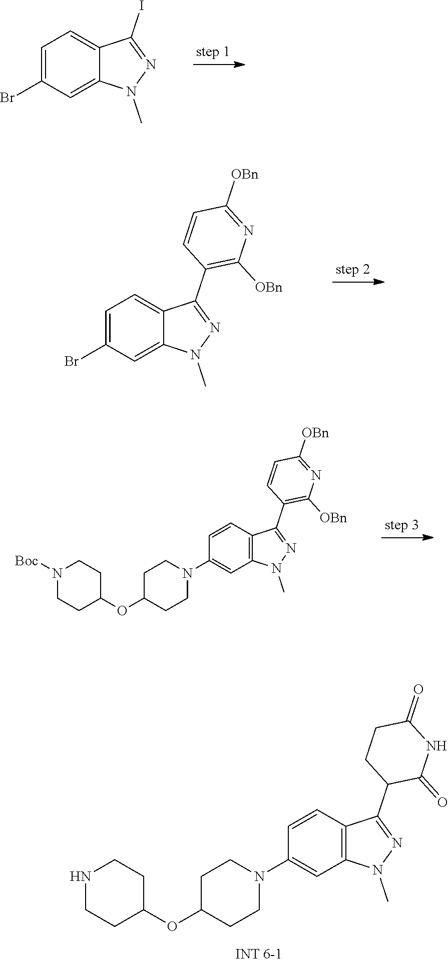 [SMILES: Cn1nc(I)c2ccc(Br)cc21 ; Cn1nc(-c2ccc(OCc3ccccc3)nc2OCc2ccccc2)c2ccc(Br)cc21 ; Cn1nc(-c2ccc(OCc3ccccc3)nc2OCc2ccccc2)c2ccc(N3CCC(OC4CCN(C(=O)OC(C)(C)C)CC4)CC3)cc21 ; Cn1nc(C2CCC(=O)NC2=O)c2ccc(N3CCC(OC4CCNCC4)CC3)cc21]

Step 1: 3-(2,6-bis(benzyloxy)pyridin-3-yl)-6-bromo-1-methyl-1H-indazole. A mixture of 6-bromo-3-iodo-1-methyl-1H-indazole (3.0 mmol), 2,6-bis(benzyloxy)-3-(4,4,5,5-tetramethyl-1,3,2-dioxaborolan-2-yl)pyridine (3.3 mmol), cesium carbonate (8.9 mmol), and [1,1′-bis(diphenylphosphino)ferrocene]dichloropalladium(II) (0.2 mmol) in 1,4-dioxane (12 mL) and water (1.5 mL) was degassed with argon for 1 minute. The reaction mixture was stirred at 80° C. for 12 hours under an argon atmosphere. The reaction mixture was then filtered through Celite and the filtrate was concentrated under reduced pressure. The residue was purified by flash column chromatography on silica gel eluting with ethyl acetate/hexanes to afford the title compound. LCMS: 347.8.

Step 2: tert-butyl 4-((1-(3-(2,6-bis(benzyloxy)pyridin-3-yl)-1-methyl-1H-indazol-6-yl)piperidin-4-yl)oxy)piperidine-1-carboxylate. A mixture of 3-(2,6-bis(benzyloxy)pyridin-3-yl)-6-bromo-1-methyl-1H-indazole (0.6 mmol), tert-butyl 4-(piperidin-4-yloxy)piperidine-1-carboxylate (0.7 mmol), 2-dicyclohexylphosphino-2′,4′,6′-triisopropylbiphenyl (0.2 mmol), cesium carbonate (1.2 mmol), and palladium acetate (0.1 mmol) in 1,4-dioxane (10 mL) was degassed with argon for 1 minute. The reaction mixture was stirred at 100° C. for 16 hours under an argon atmosphere. The reaction mixture was filtered through Celite and the filtrate was concentrated under reduced pressure. The residue was purified by flash column chromatography eluting with ethyl acetate/hexanes to afford the title compound. LCMS: 704.0.

Step 3: 3-(1-methyl-6-(4-(piperidin-4-yloxy)piperidin-1-yl)-1H-indazol-3-yl)piperidine-2,6-dione (INT 6-1). To a solution of tert-butyl 4-((1-(3-(2,6-bis(benzyloxy)pyridin-3-yl)-1-methyl-1H-indazol-6-yl)piperidin-4-yl)oxy)piperidine-1-carboxylate (0.4 mmol) in tetrahydrofuran (6 mL) was added palladium hydroxide on carbon (2.0 mmol, 20% wt Pd). The reaction mixture was evacuated and backfilled with hydrogen three times and stirred at room temperature for 16 hours. The reaction mixture was filtered through Celite and the filtrate was concentrated under reduced pressure. The residue was dissolved in dichloromethane (3 mL) and trifluoroacetic acid (9.9 mmol) was added at room temperature and the reaction mixture stirred for 1 hour. The reaction mixture was concentrated and purified by RP-HPLC (0 to 90%, 0.1% trifluoroacetic acid in acetonitrile/0.1% trifluoroacetic acid in water). Fractions containing the product were pooled and lyophilized to afford the title compound. LCMS: 426.1.

The following intermediates were made in a similar fashion to INT 6-1 and are shown below in Table 2F. To prepare the below intermediates, different reagents/starting materials were used than previously described in the steps toward INT 6-1 and are noted in the last column of Table 2F—“Changes to Procedure: Different Reagents/Starting Materials”. A person of ordinary skill in the art will readily recognize which reagents/starting materials used for the synthesis of INT 6-1 were replaced with the different reagents/starting materials noted below.

TABLE 2F
Intermediates

| | | | Changes to Procedure: Different |
|---|---|---|---|
| Intermediate | Structure | LCMS | Reagents/Starting Materials |
| INT 6-2 | 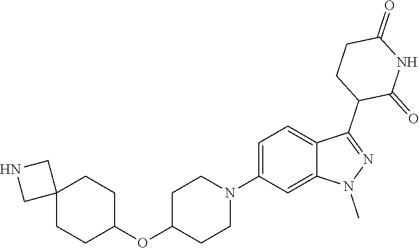 [SMILES: Cn1nc(C2CCC(=O)NC2=O)c2ccc(N3CCC(OC4CCC5(CC4)CNC5)CC3)cc21] | 466.0. | tert-butyl 7-(piperidin-4-yloxy)-2- azaspiro[3.5]nonane-2-carboxylate in step 2 |
| INT 6-3 | 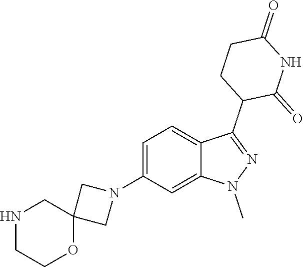 [SMILES: Cn1nc(C2CCC(=O)NC2=O)c2ccc(N3CC4(CNCCO4)C3)cc21] | 370.0 | tert-butyl 5-oxa-2,8- diazaspiro[3.5]nonane-8-carboxylate in step 2 |
| INT 6-4 | 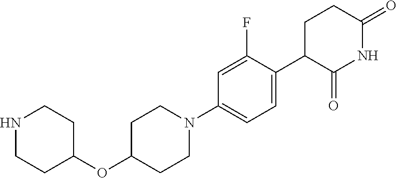 [SMILES: O=C1CCC(c2ccc(N3CCC(OC4CCNCC4)CC3)cc2F)C(=O)N1] | 390.0 | 4-bromo-2-fluoro-1-iodobenzene in step 1, replacing 6-bromo-3-iodo-1- methyl-1H-indazole |
| INT 6-5 | 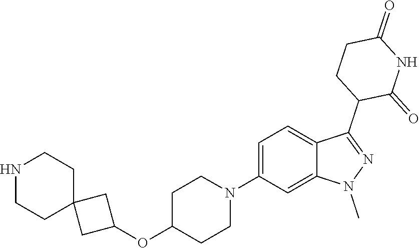 [SMILES: Cn1nc(C2CCC(=O)NC2=O)c2ccc(N3CCC(OC4CC5(CCNCC5)C4)CC3)cc21] | 466.0 | tert-butyl 2-(piperidin-4-yloxy)-7- azaspiro[3.5]nonane-7-carboxylate in step 2 |

### Intermediates 7-1-7-2.

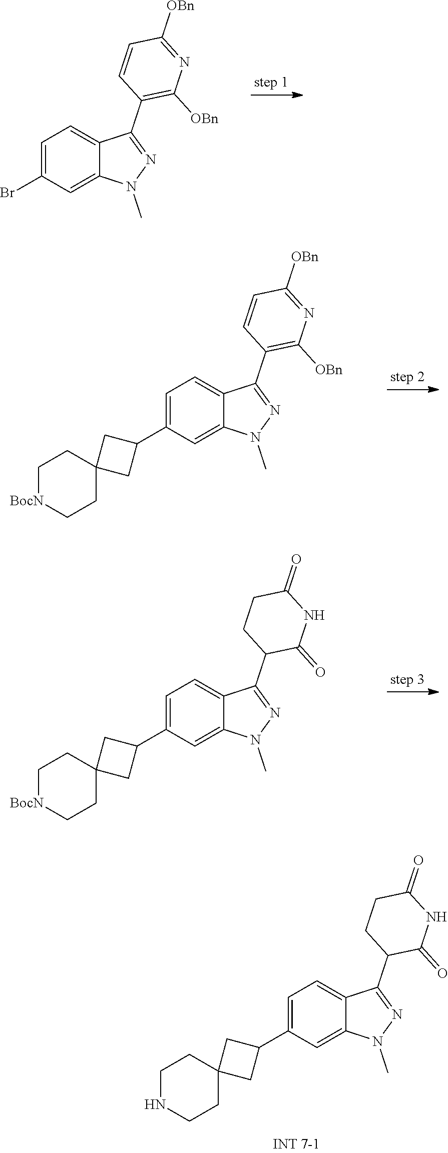 [SMILES: Cn1nc(-c2ccc(OCc3ccccc3)nc2OCc2ccccc2)c2ccc(Br)cc21 ; Cn1nc(-c2ccc(OCc3ccccc3)nc2OCc2ccccc2)c2ccc(C3CC4(CCN(C(=O)OC(C)(C)C)CC4)C3)cc21 ; Cn1nc(C2CCC(=O)NC2=O)c2ccc(C3CC4(CCN(C(=O)OC(C)(C)C)CC4)C3)cc21 ; Cn1nc(C2CCC(=O)NC2=O)c2ccc(C3CC4(CCNCC4)C3)cc21]

Step 1: Tert-butyl 2-(3-(2,6-bis(benzyloxy)pyridin-3-yl)-1-methyl-1H-indazol-6-yl)-7-azaspiro[3.5]nonane-7-carboxylate. To a vial were added 3-(2,6-bis(benzyloxy)pyridin-3-yl)-6-bromo-1-methyl-1H-indazole (0.6 mmol), tert-butyl 2-iodo-7-azaspiro[3.5]nonane-7-carboxylate (1.2 mmol), (nickel(II)[tetraaqua[4,4′-bis(1,1-dimethylethyl)-2,2′-bipyridine-κN1,κN1′]-chloride] (0.07 mmol), zinc dust (3.6 mmol), tetrabutylammonium iodide (0.7 mmol) and N,N-dimethylacetamide (2 mL). The reaction mixture was degassed with argon for 1 minute, sealed and stirred at room temperature overnight. The reaction mixture was loaded on a loading column and purified by flash chromatography eluting with ethyl acetate/hexanes to afford the title compound. LCMS: 645.9.

Step 2: tert-butyl 2-(3-(2,6-dioxopiperidin-3-yl)-1-methyl-1H-indazol-6-yl)-7-azaspiro[3.5]nonane-7-carboxylate. To a solution of tert-butyl 2-(3-(2,6-bis(benzyloxy)pyridin-3-yl)-1-methyl-H-indazol-6-yl)-7-azaspiro[3.5]nonane-7-carboxylate (0.3 mmol) in tetrahydrofuran (5 mL) was added palladium hydroxide on carbon (2.5 mmol, 20 wt % Pd). The reaction mixture was evacuated and backfilled with hydrogen three times and stirred at room temperature overnight under a hydrogen atmosphere (1 atm). The reaction mixture was filtered and the filtrate was concentrated under reduced pressure to give the title compound and was used without purification. LCMS: 467.1.

Step 3: 3-(1-methyl-6-(7-azaspiro[3.5]nonan-2-yl)-1H-indazol-3-yl)piperidine-2,6-dione (INT 7-1). To a solution of tert-butyl 2-(3-(2,6-dioxopiperidin-3-yl)-1-methyl-1H-indazol-6-yl)-7-azaspiro[3.5]nonane-7-carboxylate (0.3 mmol) in dichloromethane (6 mL) was added trifluoroacetic acid (10.1 mmol) and the reaction mixture was stirred for 1 hour. The reaction mixture was concentrated and purified by RP-HPLC (0 to 90% 0.1% TFA in MeCN/0.1% TFA in H₂O). Fractions containing the product were pooled and lyophilized to afford the title compound. LCMS: 367.0.

The following intermediates were made in a similar fashion to INT 7-1 and are shown below in Table 2G. To prepare the below intermediates, different reagents/starting materials were used than previously described in the steps toward INT 7-1 and are noted in the last column of Table 2G—“Changes to Procedure: Different Reagents/Starting Materials”. A person of ordinary skill in the art will readily recognize which reagents/starting materials used for the synthesis of INT 7-1 were replaced with the different reagents/starting materials noted below.

TABLE 2G
Intermediates

| | | | Changes to Procedure: Different |
|---|---|---|---|
| Intermediate | Structure | LCMS | Reagents/Starting Materials |
| INT 7-2 | 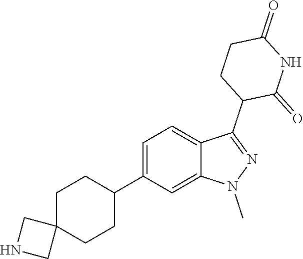 [SMILES: Cn1nc(C2CCC(=O)NC2=O)c2ccc(C3CCC4(CC3)CNC4)cc21] | 466.0. | tert-butyl 7-(piperidin-4-yloxy)-2- azaspiro[3.5]nonane-2-carboxylate in step 2, which was prepared according to this procedure: A suspension of tert- butyl 7-hydroxy-2- azaspiro[3.5]nonane-2-carboxylate (8.3 mmol), triphenylphosphine (12.4 mmol), iodine (8.3 mmol), and imidazole (16.6 mmol) in toluene (60 mL) was refluxed at 120° C. overnight. The reaction mixture was cooled down to room temperature, decanted, and purified by flash chromatography eluting with ethyl acetate/hexanes to afford tert-butyl 7-(piperidin-4-yloxy)- |
| | | | 2-azaspiro[3.5]nonane-2-carboxylate. |
| | | | LCMS: 295.9. |

### Intermediates 8-1-8-2.

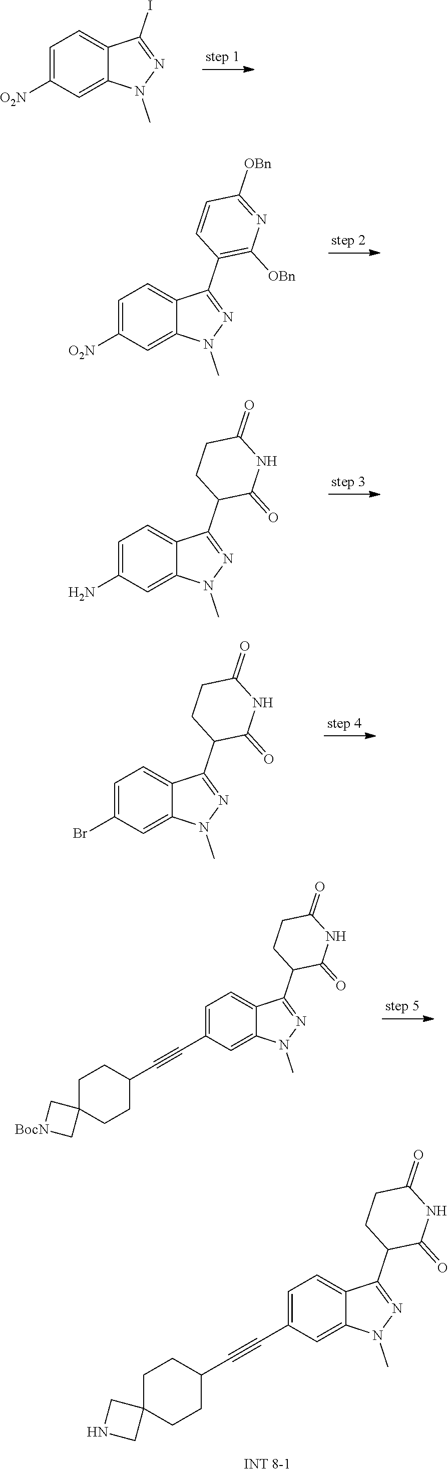

Step 1: 3-(2,6-bis(benzyloxy)pyridin-3-yl)-1-methyl-6-nitro-1H-indazole. To a stirred solution of 3-iodo-1-methyl-6-nitro-1H-indazole (83 mmol) and 2,6-bis(benzyloxy)-3-(4,4,5,5-tetramethyl-1,3,2-dioxaborolan-2-yl)pyridine (165 mmol) in 1,4-dioxane (250 mL), was added cesium carbonate (250 mmol) and water (75 mL) at room temperature. The resulting reaction mixture was degassed with argon for 15 minutes. [1,1′-bis(diphenylphosphino)ferrocene]dichloropalladium(II) (4.1 mmol) was added to the reaction mixture under argon and was degassed for another 5 minutes. The reaction mixture was heated to 100° C. and stirred for 6 hours. After completion, the reaction mixture was cooled to room temperature, filtered through a pad of Celite, and washed with ethyl acetate. The combined filtrate was washed with water, dried over sodium sulfate and concentrated under reduced pressure. The residue was purified by flash column chromatography on silica gel eluting with ethyl acetate/hexanes to afford the title compound. LCMS: 467.7.

Step 2: 3-(6-amino-1-methyl-1H-indazol-3-yl)piperidine-2,6-dione. To a stirred solution of 3-(2,6-bis(benzyloxy)pyridin-3-yl)-1-methyl-6-nitro-1H-indazole (64 mmol) in tetrahydrofuran (150 mL) and ethanol (300 mL) in a Parr apparatus was added palladium on carbon (15 g, 50 wt % Pd). The reaction mixture was stirred at room temperature for 30 hours under a hydrogen atmosphere (70 psi). After completion, the reaction mixture was filtered through a pad of Celite, and washed with ethanol and tetrahydrofuran. The combined filtrate was concentrated under reduced pressure to afford the crude material, which was purified by triturating with methyl tert-butyl ether to afford the title compound. LCMS: 467.7.

Step 3: 3-(6-bromo-1-methyl-1H-indazol-3-yl)piperidine-2,6-dione. To a stirred suspension of 3-(6-amino-1-methyl-1H-indazol-3-yl)piperidine-2,6-dione (19.3 mmol) in methanol (50 mL), was added hydrobromic acid (20 mL, 48 wt % in water) at room temperature. The reaction mixture was cooled to −5° C. and a sodium nitrite (23.2 mmol) solution in water (2.0 mL) was added dropwise and the mixture was stirred for 1 hour. Copper (I) bromide (29.0 mmol) was added at 0-5° C. and the reaction mixture was allowed to stir at room temperature for an additional hour. The reaction mixture was then poured into cold water, extracted with ethyl acetate, dried over anhydrous sodium sulfate, filtered, and concentrated under reduced pressure. The residue was purified by flash column chromatography on silica gel eluting with ethyl acetate/hexanes to afford the title compound. LCMS: 322.0.

Step 4: Tert-butyl 2-(3-(2,6-bis(benzyloxy)pyridin-3-yl)-1-methyl-1H-indazol-6-yl)-7-azaspiro[3.5]nonane-7-carboxylate. To a vial were added 3-(6-bromo-1-methyl-1H-indazol-3-yl)piperidine-2,6-dione (0.3 mmol), tert-butyl 7-ethynyl-2-azaspiro[3.5]nonane-2-carboxylate (0.5 mmol), bis(triphenylphosphine) palladium (II) dichloride (0.03 mmol), cuprous iodide (0.03 mmol), triethylamine (0.3 mmol) and 2-methyltetrahydrofuran (3 mL). The reaction mixture was degassed with argon for 10 minutes, capped and stirred at 80° C. overnight. The reaction mixture was cooled down to room temperature, filtered, concentrated and purified by flash column chromatography eluting with ethyl acetate/hexanes to afford the title compound. LCMS: 435.0.

Step 5: tert-butyl 2-(3-(2,6-bis(benzyloxy)pyridin-3-yl)-1-methyl-1H-indazol-6-yl)-7-azaspiro[3.5]nonane-7-carboxylate (INT 8-1). To a solution of 2-(3-(2,6-bis(benzyloxy)pyridin-3-yl)-1-methyl-1H-indazol-6-yl)-7-azaspiro[3.5]nonane-7-carboxylate (0.1 mmol) in dichloromethane (3 mL) was added trifluoroacetic acid (3.1 mmol) and the reaction mixture was stirred for 1 hour. The reaction mixture was concentrated and purified by RP-HPLC (0 to 90% 0.1% TFA in MeCN/0.1% TFA in H₂O). Fractions containing the product were pooled and lyophilized to afford the title compound. LCMS: 391.0.

The following Intermediates were made in a similar fashion to INT 8-1 and are shown below in Table 2H. To prepare the below Intermediates, different reagents/starting materials were used than previously described in the steps toward INT 8-1 and are noted in the last column of Table 2H—“Changes to Procedure: Different Reagents/Starting Materials”. A person of ordinary skill in the art will readily recognize which reagents/starting materials used for the synthesis of INT 8-1 were replaced with the different reagents/starting materials noted below.

TABLE 2H
Intermediates

| | | | Changes to Procedure: Different |
|---|---|---|---|
| Intermediate | Structure | LCMS | Reagents/Starting Materials |
| INT 8-2 | 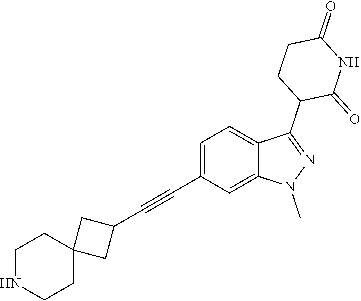 [SMILES: Cn1nc(C2CCC(=O)NC2=O)c2ccc(C#CC3CC4(CCNCC4)C3)cc21] | 390.9 | tert-butyl 2-ethynyl-7- azaspiro[3.5]nonane-7-carboxylate in step 4. |

### Intermediates 9-1-9-2.

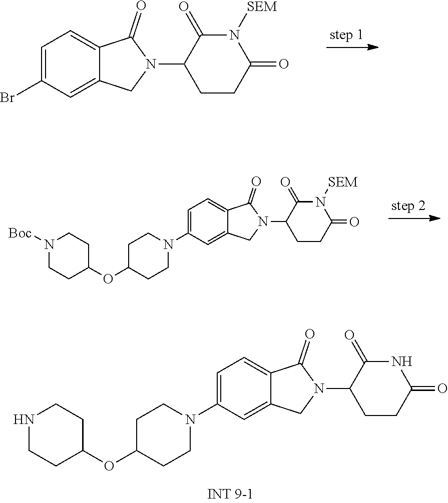 [SMILES: C[Si](C)(C)CCOCN1C(=O)CCC(N2Cc3cc(Br)ccc3C2=O)C1=O ; CC(C)(C)OC(=O)N1CCC(OC2CCN(c3ccc4c(c3)CN(C3CCC(=O)N(COCC[Si](C)(C)C)C3=O)C4=O)CC2)CC1 ; O=C1CCC(N2Cc3cc(N4CCC(OC5CCNCC5)CC4)ccc3C2=O)C(=O)N1]

Step 1: tert-butyl 4-((1-(2-(2,6-dioxo-1-((2-(trimethylsilyl)ethoxy)methyl)piperidin-3-yl)-1-oxoisoindolin-5-yl)piperidin-4-yl)oxy)piperidine-1-carboxylate. To a solution of 3-(5-bromo-1-oxoisoindolin-2-yl)-1-((2-(trimethylsilyl)ethoxy)methyl)piperidine-2,6-dione (0.4 mmol) in 1,4-dioxane (6 mL) was added tert-butyl 4-(piperidin-4-yloxy)piperidine-1-carboxylate (0.5 mmol), 2-dicyclohexylphosphino-2′,4′,6′-triisopropylbiphenyl (0.1 mmol), cesium carbonate (0.9 mmol), and palladium acetate (0.09 mmol). The resulting mixture was degassed with argon for 30 seconds and stirred at 100° C. for 16 hours under an argon atmosphere. The reaction mixture was then filtered through Celite and the filtrate was concentrated under reduced pressure. The residue was purified by flash column chromatography on silica gel by eluting with ethyl acetate/hexanes to afford the title compound. LCMS: 657.0.

Step 2: 3-(1-oxo-5-(4-(piperidin-4-yloxy)piperidin-1-yl)isoindolin-2-yl)piperidine-2,6-dione (INT 9-1). To a solution of Tert-butyl 4-((1-(2-(2,6-dioxo-1-((2-(trimethylsilyl)ethoxy)methyl)piperidin-3-yl)-1-oxoisoindolin-5-yl)piperidin-4-yl)oxy)piperidine-1-carboxylate (0.4 mmol) in dichloromethane (4 mL) was added trifluoroacetic acid (10 mmol) at room temperature. The reaction was stirred for 1 hour then concentrated under reduced pressure. The residue was then taken up in dichloromethane (4 mL) and cooled to 0° C. To the cooled solution was added triethylamine (2.9 mmol) and N,N′-dimethylethylenediamine (1.1 mmol). The reaction mixture was then stirred at room temperature for 1 hour. The reaction mixture was concentrated and purified by RP-HPLC (0 to 90%, 0.1% trifluoroacetic acid in acetonitrile/0.1% trifluoroacetic acid in water). Fractions containing the product were pooled and lyophilized to yield the title compound. LCMS: 427.0.

The following intermediates were made in a similar fashion to INT 9-1 and are shown below in Table 21. To prepare the below intermediates, different reagents/starting materials were used than previously described in the steps toward INT 9-1 and are noted in the last column of Table 21—“Changes to Procedure: Different Reagents/Starting Materials”. A person of ordinary skill in the art will readily recognize which reagents/starting materials used for the synthesis of INT 9-1 were replaced with the different reagents/starting materials noted below.

TABLE 2I
Intermediates

| | | | Changes to Procedure: Different |
|---|---|---|---|
| Intermediate | Structure | LCMS | Reagents/Starting Materials |
| INT 9-2 | 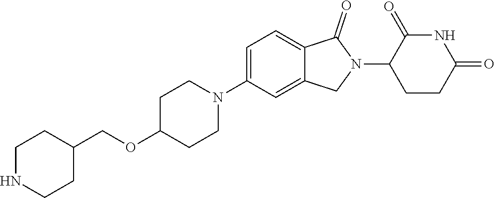 [SMILES: O=C1CCC(N2Cc3cc(N4CCC(OCC5CCNCC5)CC4)ccc3C2=O)C(=O)N1] | 441.1 | tert-butyl 4-((piperidin-4- yloxy)methyl)piperidine-1- carboxylate in step 1 |

### Intermediates 10-1-10-3.

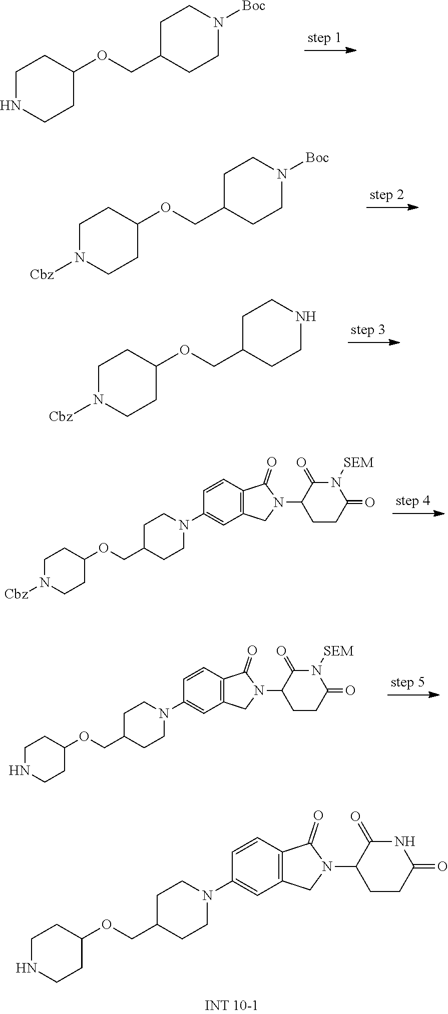 [SMILES: CC(C)(C)OC(=O)N1CCC(COC2CCNCC2)CC1 ; CC(C)(C)OC(=O)N1CCC(COC2CCN(C(=O)OCc3ccccc3)CC2)CC1 ; O=C(OCc1ccccc1)N1CCC(OCC2CCNCC2)CC1 ; C[Si](C)(C)CCOCN1C(=O)CCC(N2Cc3cc(N4CCC(COC5CCN(C(=O)OCc6ccccc6)CC5)CC4)ccc3C2=O)C1=O ; C[Si](C)(C)CCOCN1C(=O)CCC(N2Cc3cc(N4CCC(COC5CCNCC5)CC4)ccc3C2=O)C1=O ; O=C1CCC(N2Cc3cc(N4CCC(COC5CCNCC5)CC4)ccc3C2=O)C(=O)N1]

Step 1: benzyl 4-((1-(tert-butoxycarbonyl)piperidin-4-yl)methoxy)piperidine-1-carboxylate. To a solution of tert-butyl 4-((piperidin-4-yloxy)methyl)piperidine-1-carboxylate (3.0 mmol) in dichloromethane (12 mL) was added triethylamine (6.0 mmol) followed by benzyl chloroformate (2.7 mmol) at room temperature for 4 hours. Upon completion, the reaction mixture was concentrated under reduced pressure and the residue was purified by flash column chromatography on silica gel eluting with ethyl acetate/hexanes to afford the title compound. LCMS. 433.1.

Step 2: benzyl 4-(piperidin-4-ylmethoxy)piperidine-1-carboxylate. To a solution of benzyl 4-((1-(tert-butoxycarbonyl)piperidin-4-yl)methoxy)piperidine-1-carboxylate (0.8 mmol) in dichloromethane (2 mL) was added trifluoroacetic acid (22.9 mmol) at room temperature. Upon completion, the reaction mixture was concentrated under reduced pressure and the residue was purified by RP-HPLC (0 to 90%, 0.1% trifluoroacetic acid in acetonitrile/0.1% trifluoroacetic acid in water). Fractions containing the product were pooled and lyophilized to afford the title compound. LCMS: 333.0.

Step 3: benzyl 4-((1-(2-(2,6-dioxo-1-((2-(trimethylsilyl)ethoxy)methyl)piperidin-3-yl)-1-oxoisoindolin-5-yl)piperidin-4-yl)methoxy)piperidine-1-carboxylate. A mixture of benzyl 4-(piperidin-4-ylmethoxy)piperidine-1-carboxylate. To a solution of benzyl 4-((1-(tert-butoxycarbonyl)piperidin-4-yl)methoxy)piperidine-1-carboxylate (0.7 mmol), 3-(5-bromo-1-oxoisoindolin-2-yl)-1-((2-(trimethylsilyl)ethoxy)methyl)piperidine-2,6-dione (0.7 mmol), 2-dicyclohexylphosphino-2′,4′,6′-triisopropylbiphenyl (0.2 mmol), cesium carbonate (1.3 mmol), and palladium acetate (0.1 mmol) in 1,4-dioxane (9 mL) was added then degassed with argon for 1 minute. The reaction mixture was stirred at 100° C. for 16 hours under an argon atmosphere. The reaction mixture was filtered through Celite and the filtrate was concentrated under reduced pressure. The residue was purified by flash column chromatography on silica gel eluting with ethyl acetate/hexanes to afford the title compound. LCMS: 705.0.

Step 4: 3-(1-oxo-5-(4-((piperidin-4-yloxy)methyl)piperidin-1-yl)isoindolin-2-yl)-1-((2-(trimethylsilyl)ethoxy)methyl)piperidine-2,6-dione. To a solution of Benzyl 4-((1-(2-(2,6-dioxo-1-((2-(trimethylsilyl)ethoxy)methyl)piperidin-3-yl)-1-oxoisoindolin-5-yl)piperidin-4-yl)methoxy)piperidine-1-carboxylate (0.2 mmol) in tetrahydrofuran (3 mL) was added palladium hydroxide on carbon (1.0 mmol, 20% wt Pd). The reaction mixture was evacuated and backfilled with hydrogen three times and stirred at room temperature for 2 hours. The reaction mixture was filtered through Celite and the filtrate was concentrated under reduced pressure to afford the title compound without the need for further purification. LCMS: 571.1.

Step 5: 3-(1-oxo-5-(4-((piperidin-4-yloxy)methyl)piperidin-1-yl)isoindolin-2-yl)piperidine-2,6-dione (INT 10-1). To a solution of 3-(1-oxo-5-(4-((piperidin-4-yloxy)methyl)piperidin-1-yl)isoindolin-2-yl)-1-((2-(trimethylsilyl)ethoxy)methyl)piperidine-2,6-dione (0.2 mmol) in dichloromethane (3 mL) was added trifluoroacetic acid (5.6 mmol) at room temperature. The reaction was stirred for 1 hour then concentrated under reduced pressure. The residue was then taken up in dichloromethane (3 mL) and cooled to 0° C. To the cooled solution was added triethylamine (1.9 mmol) and N,N′-dimethylethylenediamine (0.3 mmol). The reaction mixture was then stirred at room temperature for 1 hour. The reaction mixture was concentrated and purified by RP-HPLC (0 to 90%, 0.1% trifluoroacetic acid in acetonitrile/0.1% trifluoroacetic acid in water). Fractions containing the product were pooled and lyophilized to yield the title compound. LCMS: 441.1.

The following intermediates were made in a similar fashion to INT 10-1 and are shown below in Table 2J. To prepare the below intermediates, different reagents/starting materials were used than previously described in the steps toward INT 10-1 and are noted in the last column of Table 2J—“Changes to Procedure: Different Reagents/Starting Materials”. A person of ordinary skill in the art will readily recognize which reagents/starting materials used for the synthesis of INT 10-1 were replaced with the different reagents/starting materials noted below.

TABLE 2J
Intermediates

| | | | Changes to Procedure: Different |
|---|---|---|---|
| Intermediate | Structure | LCMS | Reagents/Starting Materials |
| INT 10-2 | 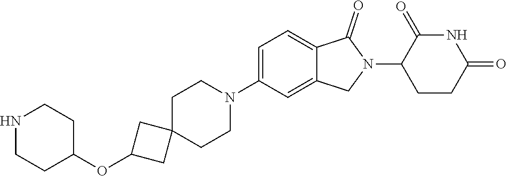 [SMILES: O=C1CCC(N2Cc3cc(N4CCC5(CC4)CC(OC4CCNCC4)C5)ccc3C2=O)C(=O)N1] | 467.0 | tert-butyl 2-(piperidin-4-yloxy)-7- azaspiro[3.5]nonane-7- carboxylate in step 1 |
| INT 10-3 | 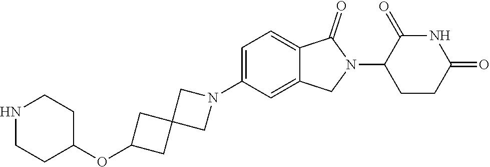 [SMILES: O=C1CCC(N2Cc3cc(N4CC5(CC(OC6CCNCC6)C5)C4)ccc3C2=O)C(=O)N1] | 438.9 | tert-butyl 6-(piperidin-4-yloxy)-2- azaspiro[3.3]heptane-2- carboxylate in step 1 |

### Intermediates 11-1-11-3.

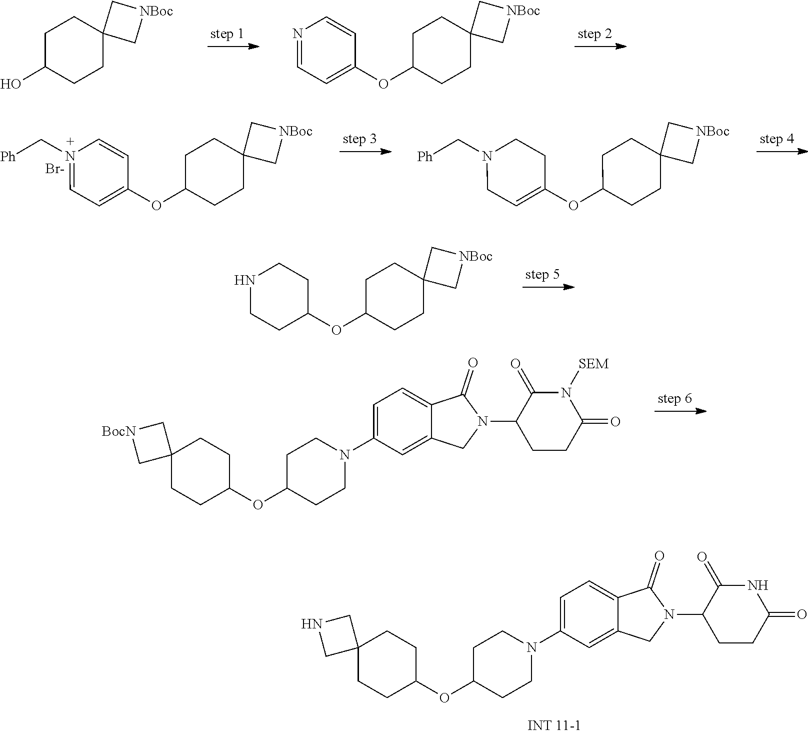

Step 1: tert-butyl 7-(pyridin-4-yloxy)-2-azaspiro[3.5]nonane-2-carboxylate. To a solution of tert-butyl 7-hydroxy-2-azaspiro[3.5]nonane-2-carboxylate (0.8 mmol) and 4-bromopyridine (0.8 mmol) in N,N-dimethylformamide (4 mL) was added a suspension of potassium tert-butoxide (16.6 mmol) in tetrahydrofuran (16 mL) dropwise. The reaction mixture was stirred at 80° C. overnight. The resulting mixture was cooled to room temperature, diluted with water (20 mL), and extracted with ethyl acetate (2×30 mL). The organic layers were combined, washed with brine (30 mL), dried over anhydrous sodium sulfate, filtered, and concentrated under reduced pressure. The residue was purified by flash column chromatography on silica gel eluting with ethyl acetate/hexanes to afford the title compound. LCMS: 319.0.

Step 2: 1-benzyl-4-((2-(tert-butoxycarbonyl)-2-azaspiro[3.5]nonan-7-yl)oxy)pyridin-1-ium bromide. To a solution of tert-butyl 7-(pyridin-4-yloxy)-2-azaspiro[3.5]nonane-2-carboxylate (0.9 mmol) in dichloromethane (3 mL) was added benzyl bromide (1.4 mmol) dropwise at 0° C. The resulting mixture was stirred at room temperature overnight. The solution was concentrated under reduced pressure. The resulting mixture was suspended in hexane and sonicated for 30 minutes. The product was filtered, washed with hexanes and dried under reduced pressure overnight to give the title compound. LCMS: 409.1.

Step 3: tert-butyl 7-((1-benzyl-1,2,3,6-tetrahydropyridin-4-yl)oxy)-2-azaspiro[3.5]nonane-2-carboxylate. To a solution of 1-benzyl-4-((2-(tert-butoxycarbonyl)-2-azaspiro[3.5]nonan-7-yl)oxy)pyridin-1-ium bromide (2.7 mmol) in methanol (10 mL) at 0° C. was added sodium borohydride (10.7 mmol) in small portions. After the cease of effervescence, the ice bath was removed and the resulting solution was stirred at room temperature for 1 hour. The solution was then concentrated, diluted with water (30 mL) and extracted with dichloromethane (3×30 mL). The organic layers were combined, washed with brine (30 mL), dried over anhydrous sodium sulfate, filtered, and concentrated under reduced pressure. The residue was purified by flash column chromatography on silica gel eluting with ethyl acetate/hexanes to afford the title compound. LCMS: 413.1.

Step 4: tert-butyl 7-(piperidin-4-yloxy)-2-azaspiro[3.5]nonane-2-carboxylate. To a solution of tert-butyl 7-((1-benzyl-1,2,3,6-tetrahydropyridin-4-yl)oxy)-2-azaspiro[3.5]nonane-2-carboxylate (2.1 mmol) in ethanol (5 mL) was added palladium hydroxide on carbon (300 mg, 20 wt % Pd) and stirred at room temperature overnight under a hydrogen atmosphere (1 atm). The resulting mixture was then filtered and concentrated to dryness under reduced pressure to provide the title compound. LCMS: 325.0.

Step 5: tert-butyl 7-((1-(2-(2,6-dioxo-1-((2-(trimethylsilyl)ethoxy)methyl)piperidin-3-yl)-1-oxoisoindolin-5-yl)piperidin-4-yl)oxy)-2-azaspiro[3.5]nonane-2-carboxylate. To a vial were added 3-(5-bromo-1-oxoisoindolin-2-yl)-1-((2-(trimethylsilyl)ethoxy)methyl)piperidine-2,6-dione (1.1 mmol), tert-butyl 7-(piperidin-4-yloxy)-2-azaspiro[3.5]nonane-2-carboxylate (1.2 mmol), 2-dicyclohexylphosphino-2′,4′,6′-triisopropylbiphenyl (0.3 mmol), cesium carbonate (2.2 mmol), palladium acetate (0.2 mmol) and 1,4-dioxane (10 mL). The resulting mixture was degassed with argon for 20 minutes, then stirred at 100° C. overnight. The reaction mixture was then filtered and the filtrate was concentrated under reduced pressure. The residue was purified by flash column chromatography on silica gel by eluting with ethyl acetate/hexanes to afford the title compound. LCMS: 697.0.

Step 6: 3-(5-(4-((2-azaspiro[3.5]nonan-7-yl)oxy)piperidin-1-yl)-1-oxoisoindolin-2-yl)piperidine-2,6-dione (INT 11-1). To a solution of tert-butyl 7-((1-(2-(2,6-dioxo-1-((2-(trimethylsilyl)ethoxy)methyl)piperidin-3-yl)-1-oxoisoindolin-5-yl)piperidin-4-yl)oxy)-2-azaspiro[3.5]nonane-2-carboxylate (0.4 mmol) in dichloromethane (4.0 mL) was added trifluoroacetic acid (10.0 mmol) at room temperature. The reaction was stirred for 1 hour then concentrated under reduced pressure. The residue was then taken up in dichloromethane (4.0 mL) and cooled to 0° C. To the cooled solution was added triethylamine (2.9 mmol) and 1,2-dimethylethylenediamine (1.1 mmol). The reaction mixture was then stirred at room temperature for 1 hour. The reaction mixture was then concentrated and purified by RP-HPLC (0 to 90%, 0.1% TFA in MeCN/0.1% TFA in H₂O). Fractions containing the product were pooled and lyophilized to yield the title compound. LCMS: 467.0.

The following intermediates were made in a similar fashion to INT 11-1 and are shown below in Table 2K. To prepare the below intermediates, different reagents/starting materials were used than previously described in the steps toward INT 11-1 and are noted in the last column of Table 2K—“Changes to Procedure: Different Reagents/Starting Materials”. A person of ordinary skill in the art will readily recognize which reagents/starting materials used for the synthesis of INT 11-1 were replaced with the different reagents/starting materials noted below.

TABLE 2K
Intermediates

| | | | Changes to Procedure: Different |
|---|---|---|---|
| Intermediate | Structure | LCMS | Reagents/Starting Materials |
| INT 11-2 | 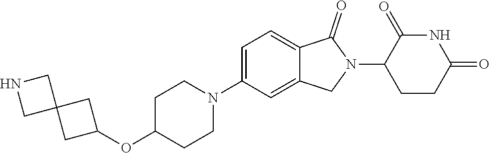 [SMILES: O=C1CCC(N2Cc3cc(N4CCC(OC5CC6(CNC6)C5)CC4)ccc3C2=O)C(=O)N1] | 439.0. | tert-butyl 6-(pyridin-4-yloxy)-2- azaspiro[3.3]heptane-2-carboxylate in step 2, was prepared according to this procedure: To a solution of tert-butyl 6- hydroxy-2-azaspiro[3.3]heptane-2- carboxylate (2.3 mmol), pyridin-4-ol (2.3 mmol) and triphenylphosphine (3.0 mmol) in tetrahydrofuran (10 mL) was added diisopropyl azodicarboxylate (3.1 mmol) dropwise at room temperature. |
| | | | The resulting reaction mixture was |
| | | | stirred at 60° C. overnight. The reaction |
| | | | mixture was concentrated under reduced |
| | | | pressure and the residue obtained was |
| | | | purified by flash column |
| | | | chromatography on silica gel eluting |
| | | | with ethyl acetate/hexanes to afford tert- |
| | | | butyl 6-(pyridin-4-yloxy)-2- |
| | | | azaspiro[3.3]heptane-2-carboxylate. |
| | | | LCMS: 291.0. |
| INT 11-3 | 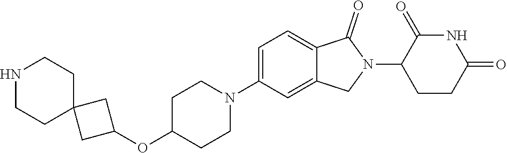 [SMILES: O=C1CCC(N2Cc3cc(N4CCC(OC5CC6(CCNCC6)C5)CC4)ccc3C2=O)C(=O)N1] | 467.0 | tert-butyl 2-(pyridin-4-yloxy)-7- azaspiro[3.5]nonane-7-carboxylate in step 2, was prepared analogously to the procedure to prepare tert-butyl 6- (pyridin-4-yloxy)-2- azaspiro[3.3]heptane-2-carboxylate in INT 11-2, except using tert-butyl 2- hydroxy-7-azaspiro[3.5]nonane-7- carboxylate. |

### Intermediate 12-1.

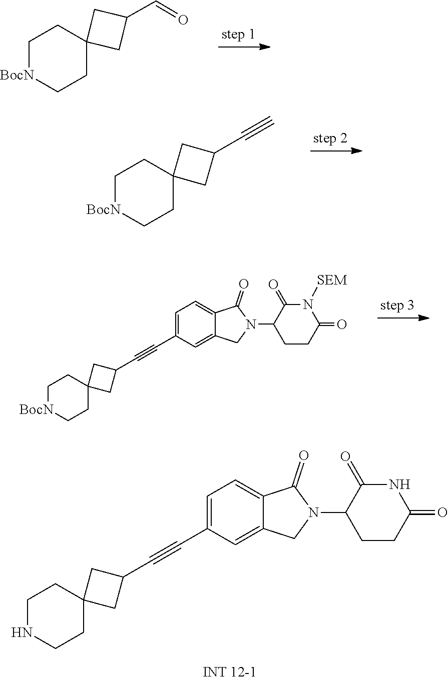 [SMILES: CC(C)(C)OC(=O)N1CCC2(CC1)CC(C=O)C2 ; C#CC1CC2(CCN(C(=O)OC(C)(C)C)CC2)C1 ; CC(C)(C)OC(=O)N1CCC2(CC1)CC(C#Cc1ccc3c(c1)CN(C1CCC(=O)N(COCC[Si](C)(C)C)C1=O)C3=O)C2 ; O=C1CCC(N2Cc3cc(C#CC4CC5(CCNCC5)C4)ccc3C2=O)C(=O)N1]

Step 1: tert-butyl 2-ethynyl-7-azaspiro[3.5]nonane-7-carboxylate. To a solution of tert-butyl 2-formyl-7-azaspiro[3.5]nonane-7-carboxylate (4.0 mmol) in methanol (16 mL), potassium carbonate (6.7 mmol) was added dimethyl (1-diazo-2-oxopropyl)phosphonate (5.0 mmol). The reaction mixture was stirred at room temperature for 5 hours. The reaction mixture was then quenched with water (15 mL) and the aqueous layer was extracted with ethyl acetate (3×20 mL). The organic layers were combined, dried over sodium sulfate, filtered, and concentrated to afford the title compound. LCMS: 194.1.

Step 2: tert-butyl 2-((2-(2,6-dioxo-1-((2-(trimethylsilyl)ethoxy)methyl)piperidin-3-yl)-1-oxoisoindolin-5-yl)ethynyl)-7-azaspiro[3.5]nonane-7-carboxylate. To a vial was added Intermediate 3-(5-bromo-1-oxoisoindolin-2-yl)-1-((2-(trimethylsilyl)ethoxy)methyl)piperidine-2,6-dione (0.4 mmol), Intermediate tert-butyl 2-ethynyl-7-azaspiro[3.5]nonane-7-carboxylate (0.9 mmol), (SP-4-3)-[dicyclohexyl[2′,4′,6′-tris(1-methylethyl)[1,1′-biphenyl]-2-yl]phosphine](methanesulfonato-κO) [2′-(methylamino-κN)[1,1′-biphenyl]-2-yl-xC]palladium (0.02 mmol), cesium carbonate (1.1 mmol) and acetonitrile (6 mL). The reaction mixture was degassed with argon for 30 seconds, capped and stirred at 60° C. for 1 hour. The reaction mixture was cooled down to room temperature, diluted with ethyl acetate (20 mL) and washed with water (2×20 mL). The organic layer was dried over anhydrous sodium sulfate, filtered, concentrated and purified by flash column chromatography eluting with ethyl acetate/hexanes to afford the title compound. LCMS: 693.9.

Step 3: 3-(5-((7-azaspiro[3.5]nonan-2-yl)ethynyl)-1-oxoisoindolin-2-yl)piperidine-2,6-dione carboxylate (INT 12-1). The title compound was prepared using the conditions described for INT 11-1, substituting tert-butyl 7-((1-(2-(2,6-dioxo-1-((2-(trimethylsilyl)ethoxy)methyl)piperidin-3-yl)-1-oxoisoindolin-5-yl)piperidin-4-yl)oxy)-2-azaspiro[3.5]nonane-2-carboxylate with tert-butyl 2-((2-(2,6-dioxo-1-((2-(trimethylsilyl)ethoxy)methyl)piperidin-3-yl)-1-oxoisoindolin-5-yl)ethynyl)-7-azaspiro[3.5]nonane-7-carboxylate in step 6. LCMS: 392.0.

### Intermediate 13-1.

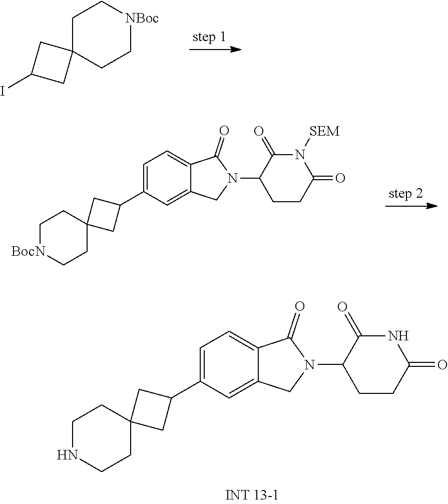 [SMILES: CC(C)(C)OC(=O)N1CCC2(CC1)CC(I)C2 ; CC(C)(C)OC(=O)N1CCC2(CC1)CC(c1ccc3c(c1)CN(C1CCC(=O)N(COCC[Si](C)(C)C)C1=O)C3=O)C2 ; O=C1CCC(N2Cc3cc(C4CC5(CCNCC5)C4)ccc3C2=O)C(=O)N1]

Step 1: tert-butyl 2-(2-(2,6-dioxo-1-((2-(trimethylsilyl)ethoxy)methyl)piperidin-3-yl)-1-oxoisoindolin-5-yl)-7-azaspiro[3.5]nonane-7-carboxylate. To a vial were added 3-(5-bromo-1-oxoisoindolin-2-yl)-1-((2-(trimethylsilyl)ethoxy)methyl)piperidine-2,6-dione (0.44 mmol), tert-butyl 2-iodo-7-azaspiro[3.5]nonane-7-carboxylate (0.88 mmol), nickel(II)[tetraaqua[4,4′-bis(1,1-dimethylethyl)-2,2′-bipyridine-κN1,κN1′]-chloride] (0.05 mmol), zinc dust (2.6 mmol), tetrabutylammonium iodide (0.5 mmol) and dimethylacetamide (1 mL). The reaction mixture was degassed with argon for 1 minute, sealed and stirred at room temperature overnight. The reaction mixture was loaded on a loading column and purified by flash chromatography eluting with ethyl acetate/hexanes to afford the title compound. LCMS: 670.0.

Step 2: 3-(1-oxo-5-(7-azaspiro[3.5]nonan-2-yl)isoindolin-2-yl)piperidine-2,6-dione (INT 13-1). To a solution of tert-butyl 2-(2-(2,6-dioxo-1-((2-(trimethylsilyl)ethoxy)methyl)piperidin-3-yl)-1-oxoisoindolin-5-yl)-7-azaspiro[3.5]nonane-7-carboxylate (0.4 mmol) in dichloromethane (4.0 mL) was added trifluoroacetic acid (10.0 mmol) at room temperature. The reaction was stirred for 1 hour then concentrated under reduced pressure. The residue was then taken up in dichloromethane (4.0 mL) and cooled to 0° C. To the cooled solution was added triethylamine (2.9 mmol) and 1,2-dimethylethylenediamine (1.1 mmol). The reaction mixture was then stirred at room temperature for 1 hour. The reaction mixture was then concentrated and purified by RP-HPLC (0 to 90%, 0.1% TFA in MeCN/0.1% TFA in H₂O). Fractions containing the product were pooled and lyophilized to yield the title compound. LCMS: 367.9.

### Intermediates 14-1-14-11.

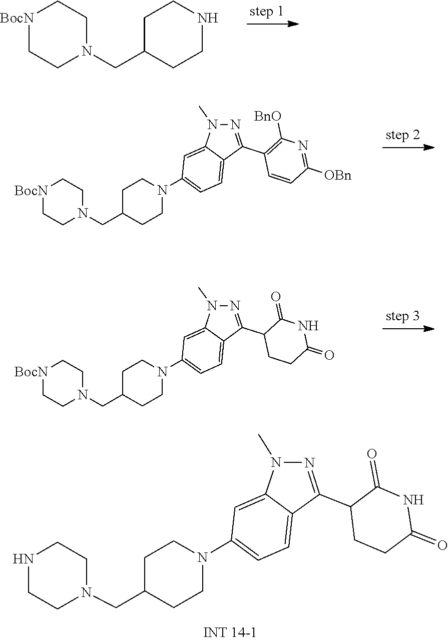 [SMILES: CC(C)(C)OC(=O)N1CCN(CC2CCNCC2)CC1 ; Cn1nc(-c2ccc(OCc3ccccc3)nc2OCc2ccccc2)c2ccc(N3CCC(CN4CCN(C(=O)OC(C)(C)C)CC4)CC3)cc21 ; Cn1nc(C2CCC(=O)NC2=O)c2ccc(N3CCC(CN4CCN(C(=O)OC(C)(C)C)CC4)CC3)cc21 ; Cn1nc(C2CCC(=O)NC2=O)c2ccc(N3CCC(CN4CCNCC4)CC3)cc21]

Step 1: tert-butyl 4-((1-(3-(2,6-bis(benzyloxy)pyridin-3-yl)-1-methyl-1H-indazol-6-yl)piperidin-4-yl)methyl)piperazine-1-carboxylate. A vigorously stirred solution of tert-butyl 4-(piperidin-4-ylmethyl)piperazine-1-carboxylate (0.8 mmol), 6-bromo-3-(2,6-dibenzyloxy-3-pyridyl)-1-methyl-indazole (0.6 mmol), tris(dibenzylideneacetone)dipalladium(0) (0.012 mmol), 2-dicyclohexylphosphino-2′,4′,6′-triisopropylbiphenyl (0.018 mmol), cesium carbonate (1.8 mmol) in 1,4-dioxane (6 mL) was heated under argon at 100° C. for 16 hours. The reaction mixture was filtered through a short pad of Celite and washed with dichloromethane (3×10 mL), then concentrated under reduced pressure. The residue was purified by flash column chromatography on silica gel (100% ethyl acetate) to afford the title compound. LCMS: 703.3.

Step 2: tert-butyl 4-((1-(3-(2,6-dioxopiperidin-3-yl)-1-methyl-1H-indazol-6-yl)piperidin-4-yl)methyl)piperazine-1-carboxylate. A mixture of palladium hydroxide on carbon (0.79 mmol, 20% wt Pd) and tert-butyl 4-((1-(3-(2,6-bis(benzyloxy)pyridin-3-yl)-1-methyl-1H-indazol-6-yl)piperidin-4-yl)methyl)piperazine-1-carboxylate (0.16 mmol) in tetrahydrofuran (5 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×10 mL), then concentrated under reduced pressure. The residue was purified by flash column chromatography on silica gel (0% to 50% ethyl acetate in hexanes) to afford the title compound. LCMS: 525.1.

Step 3: 3-(1-methyl-6-(4-(piperazin-1-ylmethyl)piperidin-1-yl)-1H-indazol-3-yl)piperidine-2,6-dione (INT 14-1). Trifluoroacetic acid (0.5 mL) was added into a solution of tert-butyl 4-((1-(3-(2,6-dioxopiperidin-3-yl)-1-methyl-1H-indazol-6-yl)piperidin-4-yl)methyl)piperazine-1-carboxylate (0.1 mmol) in dichloromethane (5 mL) at room temperature. The resulting solution was stirred at room temperature for 2 hours. Upon completion, the solution was concentrated under reduced pressure and used for the next step without further purification. LCMS: 425.3.

The following intermediates were made in a similar fashion to INT 14-1 and are shown below in Table 2L. To prepare the below intermediates, different reagents/starting materials were used than previously described in the steps toward INT 14-1 and are noted in the last column of Table 2L—“Changes to Procedure: Different Reagents/Starting Materials”. A person of ordinary skill in the art will readily recognize which reagents/starting materials used for the synthesis of INT 14-1 were replaced with the different reagents/starting materials noted below.

TABLE 2L
Intermediates

| | | | Changes to Procedure: Different |
|---|---|---|---|
| Intermediate | Structure | LCMS | Reagents/Starting Materials |
| 14-2 | 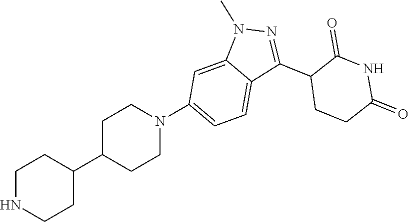 [SMILES: Cn1nc(C2CCC(=O)NC2=O)c2ccc(N3CCC(C4CCNCC4)CC3)cc21] | 410.3 | tert-butyl [4,4′-bipiperidine]-1- carboxylate in step 1 |
| 14-3 | 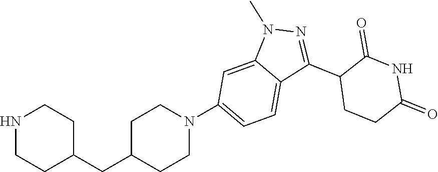 [SMILES: Cn1nc(C2CCC(=O)NC2=O)c2ccc(N3CCC(CC4CCNCC4)CC3)cc21] | 424.3 | tert-butyl 4-(piperidin-4- ylmethyl)piperidine-1-carboxylate in step 1 |
| 14-4 | 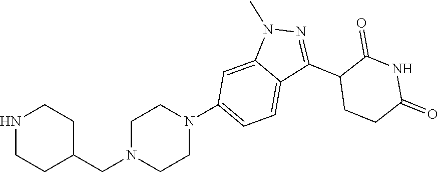 [SMILES: Cn1nc(C2CCC(=O)NC2=O)c2ccc(N3CCN(CC4CCNCC4)CC3)cc21] | 425.3 | tert-butyl 4-(piperazin-1- ylmethyl)piperidine-1-carboxylate in step 1 |
| 14-5 | 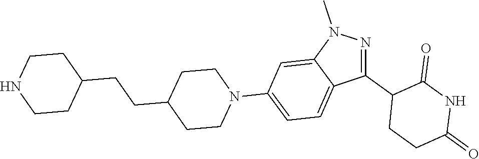 [SMILES: Cn1nc(C2CCC(=O)NC2=O)c2ccc(N3CCC(CCC4CCNCC4)CC3)cc21] | 438.3 | tert-butyl 4-[2-(piperidin-4- yl)ethyl]piperidine-1-carboxylate in step 1 |
| 14-6 | 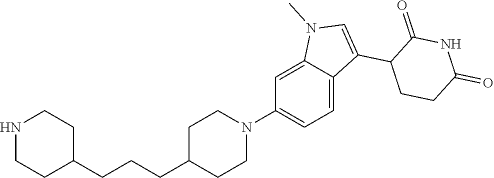 [SMILES: Cn1cc(C2CCC(=O)NC2=O)c2ccc(N3CCC(CCCC4CCNCC4)CC3)cc21] | 452.3 | tert-butyl 4-(3-(piperidin-4- yl)propyl)piperidine-1-carboxylate used in step 1 |
| 14-7 | 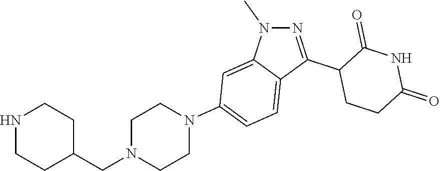 [SMILES: Cn1nc(C2CCC(=O)NC2=O)c2ccc(N3CCN(CC4CCNCC4)CC3)cc21] | 425.3 | tert-butyl 4-(piperazin-1- ylmethyl)piperidine-1-carboxylate in step 1 |
| 14-8 | 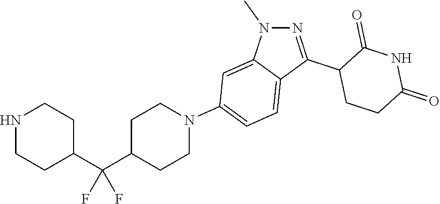 [SMILES: Cn1nc(C2CCC(=O)NC2=O)c2ccc(N3CCC(C(F)(F)C4CCNCC4)CC3)cc21] | 460.3 | tert-butyl 4-(difluoro(piperidin-4- yl)methyl)piperidine-1-carboxylate in step 1 |
| 14-9 | 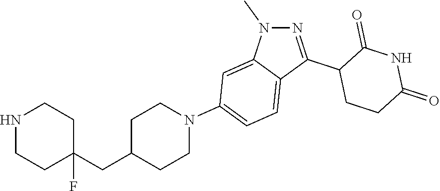 [SMILES: Cn1nc(C2CCC(=O)NC2=O)c2ccc(N3CCC(CC4(F)CCNCC4)CC3)cc21] | 442.4 | tert-butyl 4-fluoro-4-(piperidin-4- ylmethyl)piperidine-1-carboxylate in step 1 |
| 14-10 | 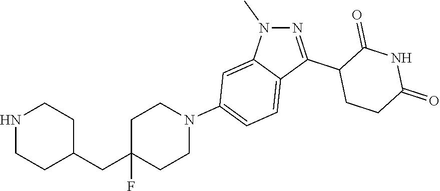 [SMILES: Cn1nc(C2CCC(=O)NC2=O)c2ccc(N3CCC(F)(CC4CCNCC4)CC3)cc21] | 442.3 | tert-butyl 4-((4-fluoropiperidin-4- yl)methyl)piperidine-1-carboxylate in step 1 |
| 14-11 | 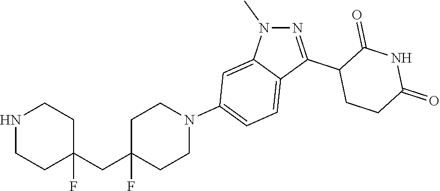 [SMILES: Cn1nc(C2CCC(=O)NC2=O)c2ccc(N3CCC(F)(CC4(F)CCNCC4)CC3)cc21] | 460.3 | tert-butyl 4-fluoro-4-((4- fluoropiperidin-4-yl)methyl)piperidine- 1-carboxylate in step 1 |

### Intermediate 15-1.

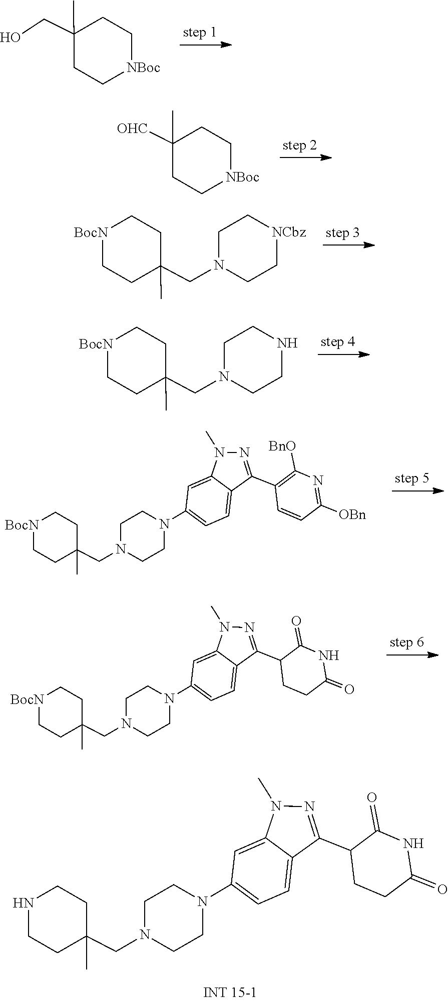

Step 1: tert-butyl 4-formyl-4-methylpiperidine-1-carboxylate. To a solution of tert-butyl 4-(hydroxymethyl)-4-methylpiperidine-1-carboxylate (8.7 mmol) in dichloromethane (25 mL) was slowly added pyridinium chlorochromate (26 mmol) at ° C. The reaction mixture was allowed to warm to room temperature and stirred for 2 hours, then diluted with 50 mL water. The mixture was extracted by 3×50 mL dichloromethane, then concentrated under reduced pressure. The residue was purified by flash column chromatography on silica gel (0% to 10% ethyl acetate in hexanes) to afford the title compound. ¹H NMR (400 MHz, Chloroform-d) δ 9.46 (s, 1H), 3.70-3.58 (m, 2H), 3.11 (ddd, J=13.5, 9.8, 3.5 Hz, 2H), 1.90 (dddd, J=13.6, 5.2, 3.5, 1.1 Hz, 2H), 1.45 (s, 9H), 1.40 (ddd, J=13.9, 9.8, 4.2 Hz, 2H), 1.08 (s, 3H).

Step 2: benzyl 4-((1-(tert-butoxycarbonyl)-4-methylpiperidin-4-yl)methyl)piperazine-1-carboxylate. To a mixture of tert-butyl 4-formyl-4-methyl-piperidine-1-carboxylate (4.4 mmol), benzyl piperazine-1-carboxylate (4.4 mmol) and acetic acid (4.4 mmol) in 1,2-dichloroethane (10 mL) was slowly added sodium triacetoxyborohydride (8.8 mmol). The reaction mixture was vigorously stirred at room temperature for 48 hours. Upon completion, water (10 mL) and saturated NaHCO₃ solution (10 mL) were added sequentially. The organic layer was separated, dried over anhydrous magnesium sulfate, filtered, and concentrated under reduced pressure. The residue was purified by flash column chromatography on silica gel (0% to 30% ethyl acetate in hexanes) to give the title compound. LCMS: 432.3.

Step 3: tert-butyl 4-methyl-4-(piperazin-1-ylmethyl)piperidine-1-carboxylate. A mixture of palladium hydroxide on carbon (5.82 mmol, 20% wt Pd) and benzyl 4-[(1-tert-butoxycarbonyl-4-methyl-4-piperidyl)methyl]piperazine-1-carboxylate (1.9 mmol) in tetrahydrofuran (5 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×10 mL), concentrated under reduced pressure and used for the next step. LCMS: 298.3.

Step 4: tert-butyl 4-((4-(3-(2,6-bis(benzyloxy)pyridin-3-yl)-1-methyl-1H-indazol-6-yl)piperazin-1-yl)methyl)-4-methylpiperidine-1-carboxylate. A vigorously stirred solution of tert-butyl 4-methyl-4-(piperazin-1-ylmethyl)piperidine-1-carboxylate (0.81 mmol), 6-bromo-3-(2,6-dibenzyloxy-3-pyridyl)-1-methyl-indazole (0.58 mmol), tris(dibenzylideneacetone)dipalladium(0) (0.012 mmol), 2-dicyclohexylphosphino-2′,4′,6′-triisopropylbiphenyl (0.017 mmol), cesium carbonate (1.74 mmol) in 1,4-dioxane (5 mL) was heated under argon at 100° C. for 16 hours. The reaction mixture was filtered through a short pad of Celite and washed with dichloromethane (3×10 mL), then concentrated under reduced pressure. The residue was purified by flash column chromatography on silica gel (100% ethyl acetate) to afford the title compound. LCMS: 717.5.

Step 5: tert-butyl 4-((4-(3-(2,6-dioxopiperidin-3-yl)-1-methyl-1H-indazol-6-yl)piperazin-1-yl)methyl)-4-methylpiperidine-1-carboxylate. A mixture of Palladium hydroxide on carbon (2.49 mmol) and tert-butyl 4-((4-(3-(2,6-bis(benzyloxy)pyridin-3-yl)-1-methyl-1H-indazol-6-yl)piperazin-1-yl)methyl)-4-methylpiperidine-1-carboxylate (0.49 mmol) in tetrahydrofuran (5 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×10 mL), concentrated under reduced pressure, and used for the next step without further purification. LCMS: 539.4.

Step 6: 3-(1-methyl-6-(4-((4-methylpiperidin-4-yl)methyl)piperazin-1-yl)-1H-indazol-3-yl)piperidine-2,6-dione (INT 15-1). Trifluoroacetic acid (0.5 mL) was added into a solution of tert-butyl 4-[[4-[3-(2,6-dioxo-3-piperidyl)-1-methyl-indazol-6-yl]piperazin-1-yl]methyl]-4-methyl-piperidine-1-carboxylate (0.37 mmol) in dichloromethane (5 mL) at room temperature. The resulting solution was stirred for 2 hours. Upon completion, the solution was concentrated under reduced pressure and used for the next step without further purification. LCMS: 439.3.

### Intermediate 16-1.

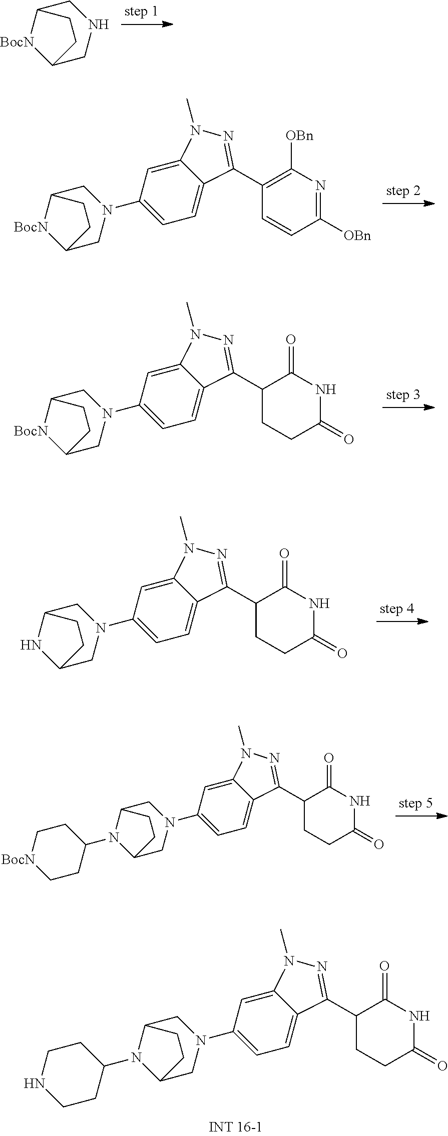 [SMILES: CC(C)(C)OC(=O)N1C2CCC1CNC2 ; Cn1nc(-c2ccc(OCc3ccccc3)nc2OCc2ccccc2)c2ccc(N3CC4CCC(C3)N4C(=O)OC(C)(C)C)cc21 ; Cn1nc(C2CCC(=O)NC2=O)c2ccc(N3CC4CCC(C3)N4C(=O)OC(C)(C)C)cc21 ; Cn1nc(C2CCC(=O)NC2=O)c2ccc(N3CC4CCC(C3)N4)cc21 ; Cn1nc(C2CCC(=O)NC2=O)c2ccc(N3CC4CCC(C3)N4C3CCN(C(=O)OC(C)(C)C)CC3)cc21 ; Cn1nc(C2CCC(=O)NC2=O)c2ccc(N3CC4CCC(C3)N4C3CCNCC3)cc21]

Step 1: tert-butyl 3-(3-(2,6-bis(benzyloxy)pyridin-3-yl)-1-methyl-1H-indazol-6-yl)-3,8-diazabicyclo[3.2.1]octane-8-carboxylate. A vigorously stirred solution of tert-butyl 3,8-diazabicyclo[3.2.1]octane-8-carboxylate (2 mmol), 6-bromo-3-(2,6-dibenzyloxy-3-pyridyl)-1-methyl-indazole (2 mmol), tris(dibenzylideneacetone)dipalladium(0) (0.04 mmol), 2-dicyclohexylphosphino-2′,4′,6′-triisopropylbiphenyl (0.06 mmol), and cesium carbonate (6 mmol) in 1,4-dioxane (15 mL) was heated under argon at 100° C. for 16 hours. The reaction mixture was filtered through a short pad of Celite and washed with dichloromethane (3×50 mL), then concentrated under reduced pressure. The residue was purified by flash column chromatography on silica gel (100% ethyl acetate) to afford the title compound. LCMS: 632.5.

Step 2: tert-butyl 3-(3-(2,6-dioxopiperidin-3-yl)-1-methyl-1H-indazol-6-yl)-3,8-diazabicyclo[3.2.1]octane-8-carboxylate. A mixture of palladium hydroxide on carbon (1.2 mmol, 20% wt Pd) and tert-butyl 3-(3-(2,6-bis(benzyloxy)pyridin-3-yl)-1-methyl-1H-indazol-6-yl)-3,8-diazabicyclo[l3.2.1]octane-8-carboxylate (1.2 mmol) in tetrahydrofuran (5 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×10 mL), concentrated under reduced pressure and used for the next step. LCMS: 454.3.

Step 3: 3-(6-(3,8-diazabicyclo[3.2.1]octan-3-yl)-1-methyl-1H-indazol-3-yl)piperidine-2,6-dione. Trifluoroacetic acid (0.5 mL) was added into a solution of tert-butyl 3-(3-(2,6-dioxopiperidin-3-yl)-1-methyl-1H-indazol-6-yl)-3, 8-diazabicyclo[3.2.1]octane-8-carboxylate (0.28 mmol) in dichloromethane (5 mL) at room temperature. The resulting solution was stirred for 2 hours. Upon completion, the solution was concentrated under reduced pressure and used for the next step without further purification. LCMS: 354.4.

Step 4: tert-butyl 4-(3-(3-(2,6-dioxopiperidin-3-yl)-1-methyl-1H-indazol-6-yl)-3,8-diazabicyclo[3.2.1]octan-8-yl)piperidine-1-carboxylate. To a mixture of 3-[6-(3,8-diazabicyclo[3.2.1]octan-3-yl)-1-methyl-indazol-3-yl]piperidine-2,6-dione (0.28 mmol), 1-Boc-4-piperidone (2.8 mmol), acetic acid (1.4 mmol), sodium acetate (1.4 mmol) in methanol (4 mL) and N,N-dimethylformamide (1 mL) was slowly added sodium cyanoborohydride (2.8 mmol) at room temperature. The reaction mixture was vigorously stirred at 50° C. for 3 hours. Upon completion, the reaction was diluted with 50 mL ethyl acetate and 50 mL brine, extracted by 3×20 mL ethyl acetate, dried over sodium sulfate, and concentrated under reduced pressure. The residue was purified by flash column chromatography on silica gel (0% to 15% methanol in dichloromethane) to give the title compound. LCMS: 537.4.

Step 5: 3-(1-methyl-6-(8-(piperidin-4-yl)-3,8-diazabicyclo[3.2.1]octan-3-yl)-1H-indazol-3-yl)piperidine-2,6-dione (INT 16-1). Trifluoroacetic acid (0.5 mL) was added into a solution of tert-butyl 4-(3-(3-(2,6-dioxopiperidin-3-yl)-1-methyl-1H-indazol-6-yl)-3,8-diazabicyclo[3.2.1]octan-8-yl)piperidine-1-carboxylate (0.37 mmol) in dichloromethane (5 mL) at room temperature. The resulting solution was stirred for 2 hours. Upon completion, the solution was concentrated under reduced pressure and used for the next step without further purification. LCMS: 437.4.

### Intermediates 17-1-17-9.

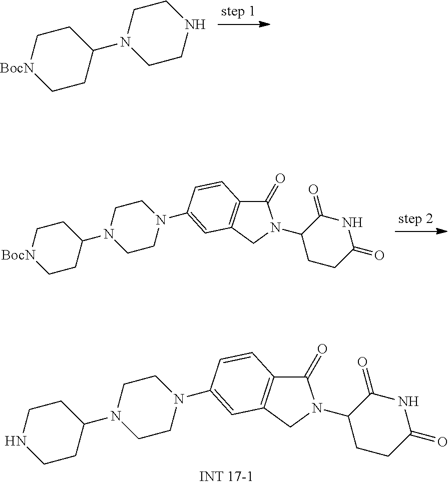 [SMILES: CC(C)(C)OC(=O)N1CCC(N2CCNCC2)CC1 ; CC(C)(C)OC(=O)N1CCC(N2CCN(c3ccc4c(c3)CN(C3CCC(=O)NC3=O)C4=O)CC2)CC1 ; O=C1CCC(N2Cc3cc(N4CCN(C5CCNCC5)CC4)ccc3C2=O)C(=O)N1]

Step 1: tert-butyl 4-(4-(2-(2,6-dioxopiperidin-3-yl)-1-oxoisoindolin-5-yl)piperazin-1-yl)piperidine-1-carboxylate. A vigorously stirred solution of tert-butyl 4-piperazin-1-ylpiperidine-1-carboxylate (2.6 mmol), 3-(5-bromo-1-oxo-isoindolin-2-yl)piperidine-2,6-dione (2.6 mmol), dichloro[1,3-bis(2,6-di-3-pentylphenyl)imidazol-2-ylidene](3-chloropyridyl)palladium(II) (0.13 mmol), and cesium carbonate (7.8 mmol) in 1,4-dioxane (15 mL) was heated under argon at 100° C. for 16 hours. The reaction mixture was filtered through a short pad of Celite and washed with dichloromethane (3×50 mL), then concentrated under reduced pressure. The residue was purified by flash column chromatography on silica gel (0% to 15% methanol in dichloromethane) to afford the title compound. LCMS: 512.4.

Step 2: 3-(1-oxo-5-(4-(piperidin-4-yl)piperazin-1-yl)isoindolin-2-yl)piperidine-2,6-dione (INT 17-1). Trifluoroacetic acid (0.5 mL) was added into a solution of tert-butyl 4-(4-(2-(2,6-dioxopiperidin-3-yl)-1-oxoisoindolin-5-yl)piperazin-1-yl)piperidine-1-carboxylate (1.4 mmol) in dichloromethane (5 mL) at room temperature. The resulting solution was stirred for 2 hours. Upon completion, the solution was concentrated under reduced pressure and used for the next step without further purification. LCMS: 412.4.

The following intermediates were made in a similar fashion to INT 17-1 and are shown below in Table 2M. To prepare the below intermediates, different reagents/starting materials were used than previously described in the steps toward INT 17-1 and are noted in the last column of Table 2M—“Changes to Procedure: Different Reagents/Starting Materials”. A person of ordinary skill in the art will readily recognize which reagents/starting materials used for the synthesis of INT 17-1 were replaced with the different reagents/starting materials noted below.

TABLE 2M
Intermediates

| | | | Changes to Procedure: Different |
|---|---|---|---|
| Intermediate | Structure | LCMS | Reagents/Starting Materials |
| 17-2 | 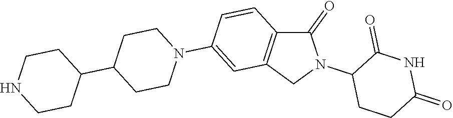 [SMILES: O=C1CCC(N2Cc3cc(N4CCC(C5CCNCC5)CC4)ccc3C2=O)C(=O)N1] | 411.3 | tert-butyl [4,4′-bipiperidine]-1- carboxylate in step 1 |
| 17-3 | 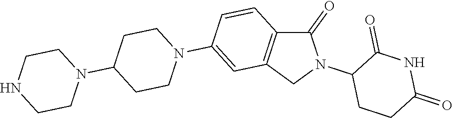 [SMILES: O=C1CCC(N2Cc3cc(N4CCC(N5CCNCC5)CC4)ccc3C2=O)C(=O)N1] | 412.3 | tert-butyl 4-(piperidin-4-yl) piperazine- 1-carboxylate in step 1 |
| 17-4 | 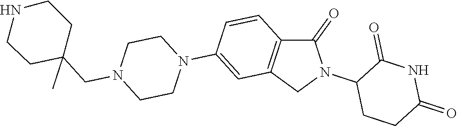 [SMILES: CC1(CN2CCN(c3ccc4c(c3)CN(C3CCC(=O)NC3=O)C4=O)CC2)CCNCC1] | 440.3 | tert-butyl 4-methyl-4-(piperazin-1- ylmethyl)piperidine-1- carboxylate in step 1 |
| 17-5 | 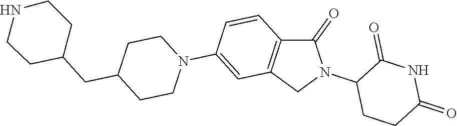 [SMILES: O=C1CCC(N2Cc3cc(N4CCC(CC5CCNCC5)CC4)ccc3C2=O)C(=O)N1] | 425.3 | tert-butyl 4-(piperidin-4- ylmethyl)piperidine-1- carboxylate in step 1 |
| 17-6 | 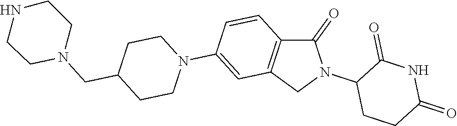 [SMILES: O=C1CCC(N2Cc3cc(N4CCC(CN5CCNCC5)CC4)ccc3C2=O)C(=O)N1] | 426.3 | tert-butyl 4-(piperidin-4- ylmethyl)piperazine-1- carboxylate in step 1 |
| 17-7 | 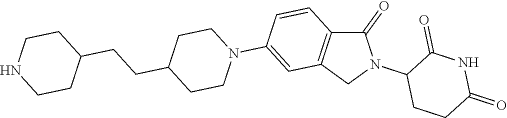 [SMILES: O=C1CCC(N2Cc3cc(N4CCC(CCC5CCNCC5)CC4)ccc3C2=O)C(=O)N1] | 439.3 | tert-butyl 4-(2-(piperidin-4- yl)ethyl)piperidine-1- carboxylate in step 1 |
| 17-8 | 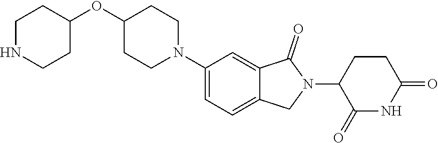 [SMILES: O=C1CCC(N2Cc3ccc(N4CCC(OC5CCNCC5)CC4)cc3C2=O)C(=O)N1] | 427.3 | 3-(6-bromo-1-oxoisoindolin-2- yl)piperidine-2,6-dione and tert- butyl 4-(piperidin-4-yloxy) piperidine-1- carboxylate in step 1 |
| 17-9 | 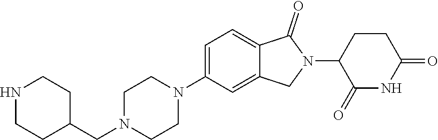 [SMILES: O=C1CCC(N2Cc3cc(N4CCN(CC5CCNCC5)CC4)ccc3C2=O)C(=O)N1] | 426.3 | tert-butyl 4-(piperazin-1- ylmethyl)piperidine-1- carboxylate in step 1 |

### Intermediate 18-1.

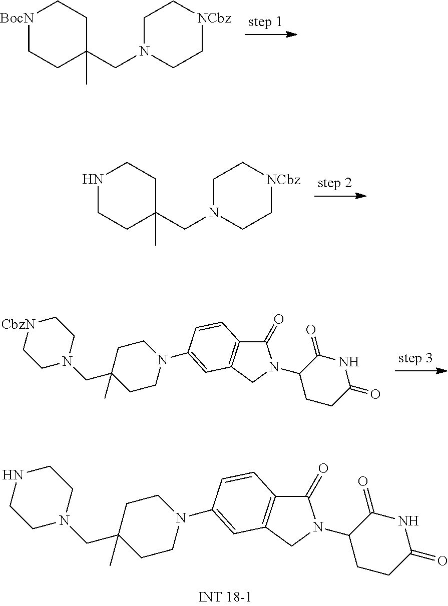 [SMILES: CC1(CN2CCN(C(=O)OCc3ccccc3)CC2)CCN(C(=O)OC(C)(C)C)CC1 ; CC1(CN2CCN(C(=O)OCc3ccccc3)CC2)CCNCC1 ; CC1(CN2CCN(C(=O)OCc3ccccc3)CC2)CCN(c2ccc3c(c2)CN(C2CCC(=O)NC2=O)C3=O)CC1 ; CC1(CN2CCNCC2)CCN(c2ccc3c(c2)CN(C2CCC(=O)NC2=O)C3=O)CC1]

Step 1: benzyl 4-((4-methylpiperidin-4-yl)methyl)piperazine-1-carboxylate. Trifluoroacetic acid (0.5 mL) was added into a solution of tert-butyl benzyl 4-((1-(tert-butoxycarbonyl)-4-methylpiperidin-4-yl)methyl)piperazine-1-carboxylate (0.5 mmol) in dichloromethane (5 mL) at room temperature. The resulting solution was stirred for 2 hours. Upon completion, the solution was concentrated under reduced pressure and used for the next step without further purification. LCMS: 332.5.

Step 2: benzyl 4-((1-(2-(2,6-dioxopiperidin-3-yl)-1-oxoisoindolin-5-yl)-4-methylpiperidin-4-yl)methyl)piperazine-1-carboxylate. A vigorously stirred solution of benzyl 4-((4-methylpiperidin-4-yl)methyl)piperazine-1-carboxylate (0.4 mmol), 3-(5-bromo-1-oxo-isoindolin-2-yl)piperidine-2,6-dione (0.4 mmol), dichloro[1,3-bis(2,6-Di-3-pentylphenyl)imidazol-2-ylidene](3-chloropyridyl)palladium(II) (0.02 mmol), and cesium carbonate (1.2 mmol) in 1,4-dioxane (5 mL) was heated under argon at 100° C. for 16 hours. The reaction mixture was filtered through a short pad of Celite and washed with dichloromethane (3×10 mL), then concentrated under reduced pressure. The residue was purified by flash column chromatography on silica gel (0% to 15% methanol in dichloromethane) to afford the title compound. LCMS: 574.4.

Step 3: 3-(5-(4-methyl-4-(piperazin-1-ylmethyl)piperidin-1-yl)-1-oxoisoindolin-2-yl)piperidine-2,6-dione (INT 18-1). A mixture of palladium hydroxide on carbon (1.2 mmol, 20% wt Pd) and benzyl 4-((1-(2-(2,6-dioxopiperidin-3-yl)-1-oxoisoindolin-5-yl)-4-methylpiperidin-4-yl)methyl)piperazine-1-carboxylate (1.2 mmol) in tetrahydrofuran (5 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×10 mL), concentrated under reduced pressure and used for the next step. LCMS: 440.3.

### Intermediate 19-1.

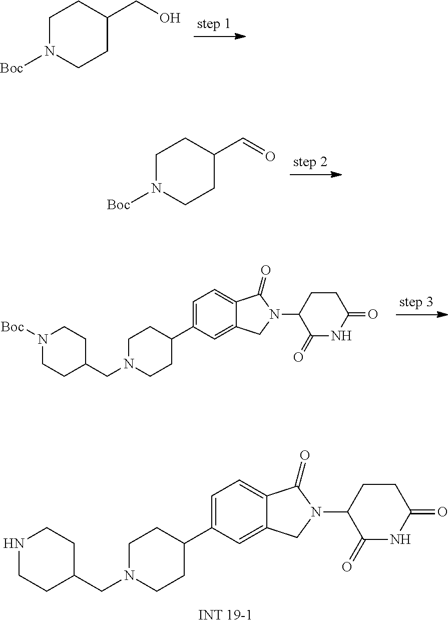 [SMILES: CC(C)(C)OC(=O)N1CCC(CO)CC1 ; CC(C)(C)OC(=O)N1CCC(C=O)CC1 ; CC(C)(C)OC(=O)N1CCC(CN2CCC(c3ccc4c(c3)CN(C3CCC(=O)NC3=O)C4=O)CC2)CC1 ; O=C1CCC(N2Cc3cc(C4CCN(CC5CCNCC5)CC4)ccc3C2=O)C(=O)N1]

Step 1: tert-butyl 4-formylpiperidine-1-carboxylate. To a stirred solution of tert-butyl 4-(hydroxymethyl)piperidine-1-carboxylate (1.2 mmol) in dichloromethane (5 mL) at 0° C. was added Dess-Martin periodinane (1.5 mmol) in one portion. The reaction mixture was stirred at 0° C. for 30 minutes, then warmed to room temperature and stirred for 3 hours. The reaction mixture was diluted with dichloromethane (20 mL), followed by saturated aqueous sodium thiosulfate (10 mL) and saturated aqueous sodium bicarbonate (10 mL). The aqueous layer was extracted with dichloromethane (3×20 mL). The combined organic layers were washed with brine (20 mL), dried over anhydrous sodium sulfate, filtered, and concentrated under reduced pressure to yield the title compound which was used without further purification.

Step 2: tert-butyl 4-((4-(2-(2,6-dioxopiperidin-3-yl)-1-oxoisoindolin-5-yl)piperidin-1-yl)methyl)piperidine-1-carboxylate. To a stirred solution of 3-(1-oxo-5-(piperidin-4-yl)isoindolin-2-yl)piperidine-2,6-dione (0.23 mmol) in methanol (1.6 mL) and N,N-dimethylformamide (0.4 mL) at room temperature was added tert-butyl 4-formylpiperidine-1-carboxylate (1.16 mmol), followed by acetic acid (33 μL). The reaction mixture was stirred at room temperature for 15 minutes, then sodium cyanoborohydride (1.16 mmol) was added. The reaction mixture was stirred at room temperature for 4 hours and was then diluted with dichloromethane (20 mL) and saturated aqueous sodium bicarbonate (20 mL). The aqueous layer was extracted with dichloromethane (3×20 mL). The combined organic extracts were washed with brine (20 mL), dried over anhydrous sodium sulfate, filtered, and concentrated under reduced pressure. The residue was purified by flash column chromatography on silica (0% to 30% methanol in dichloromethane) to yield the title compound. LCMS: 525.4.

Step 3: 3-(1-oxo-5-(1-(piperidin-4-ylmethyl)piperidin-4-yl)isoindolin-2-yl)piperidine-2,6-dione (INT 19-1). Trifluoroacetic acid (0.1 mL) was added to a stirred solution of tert-butyl 4-((4-(2-(2,6-dioxopiperidin-3-yl)-1-oxoisoindolin-5-yl)piperidin-1-yl)methyl)piperidine-1-carboxylate (0.22 mmol) in dichloromethane (2 mL) at room temperature. After stirring at room temperature overnight, the reaction mixture was diluted with 4:1 dichloromethane:isopropanol (30 mL) followed by saturated aqueous sodium bicarbonate (4 mL). The aqueous layer was extracted with 4:1 dichloromethane:isopropanol (5×30 mL). The combined organic extracts were dried over anhydrous sodium sulfate, filtered, and concentrated to yield the title compound which was used without further purification. LCMS: 425.3.

### Intermediates 20-1-20-2.

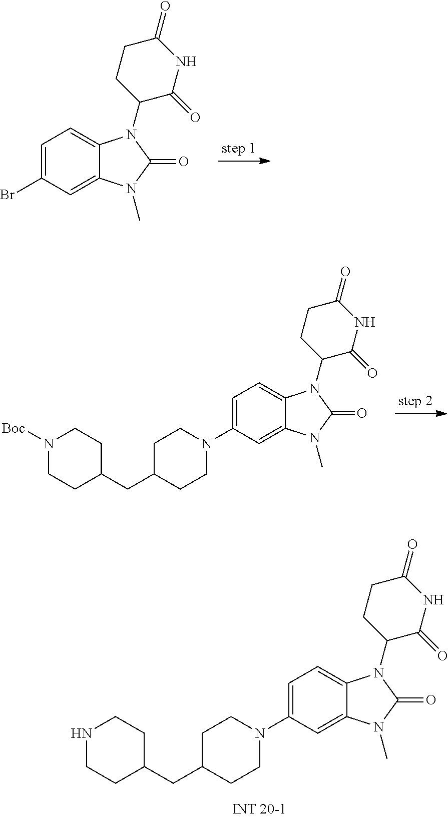 [SMILES: Cn1c(=O)n(C2CCC(=O)NC2=O)c2ccc(Br)cc21 ; Cn1c(=O)n(C2CCC(=O)NC2=O)c2ccc(N3CCC(CC4CCN(C(=O)OC(C)(C)C)CC4)CC3)cc21 ; Cn1c(=O)n(C2CCC(=O)NC2=O)c2ccc(N3CCC(CC4CCNCC4)CC3)cc21]

Step 1: tert-butyl 4-((1-(1-(2,6-dioxopiperidin-3-yl)-3-methyl-2-oxo-2,3-dihydro-1H-benzo[d]imidazol-5-yl)piperidin-4-yl)methyl)piperidine-1-carboxylate. To a mixture of 3-(5-bromo-3-methyl-2-oxo-benzimidazol-1-yl)piperidine-2,6-dione (2.4 mmol) and tert-butyl 4-(4-piperidylmethyl)piperidine-1-carboxylate (2.8 mmol) in toluene (40 mL) was added lithium bis(trimethylsilyl)amide (1.00 mol/L, 14.2 mL), 2-dicyclohexylphosphino-2′,6′-di-i-propoxy-1,1′-biphenyl (0.47 mmol), and [2-(2-aminophenyl)phenyl]-hydroxy-oxo-palladium; [2-chloro-6-(2,6-diisopropoxyphenyl)phenyl]-dicyclohexyl-phosphane (0.473 mmol). The mixture was stirred at 100° C. for 1 hour under a nitrogen atmosphere. The reaction mixture was quenched by addition of formic acid at 0° C. to adjust pH to 7, then filtered and concentrated under reduced pressure to give a residue. The residue was purified by prep-HPLC (20% to 50% acetonitrile/water with 0.1% trifluoroacetic acid), and fractions containing the desired product were pooled and lyophilized to yield the title compound. LCMS: 540.4.

Step 2: 3-(3-methyl-2-oxo-5-(4-(piperidin-4-ylmethyl)piperidin-1-yl)-2,3-dihydro-1H-benzo[d]imidazol-1-yl)piperidine-2,6-dione (INT 20-1). To a mixture of tert-butyl 4-((1-(1-(2,6-dioxopiperidin-3-yl)-3-methyl-2-oxo-2,3-dihydro-1H-benzo[d]imidazol-5-yl)piperidin-4-yl)methyl)piperidine-1-carboxylate (0.93 mmol) in ethyl acetate (4 mL) was added hydrochloric acid in dioxane (1 mL, 4 M). The mixture was stirred at room temperature for 30 minutes. The reaction mixture was concentrated under reduced pressure to give a residue. The residue was purified by prep-HPLC (1% to 15% acetonitrile/water with 0.04% hydrochloric acid). Fractions containing the desired product were lyophilized to yield the title compound. LCMS: 440.2.

The following intermediates were made in a similar fashion to INT 20-1 and are shown below in Table 2N. To prepare the below intermediates, different reagents/starting materials were used than previously described in the steps toward INT 20-1 and are noted in the last column of Table 2N—“Changes to Procedure: Different Reagents/Starting Materials”. A person of ordinary skill in the art will readily recognize which reagents/starting materials used for the synthesis of INT 20-1 were replaced with the different reagents/starting materials noted below.

TABLE 2N
Intermediates

| | | | Changes to Procedure: Different |
|---|---|---|---|
| | Intermediate | Structure | LCMS |
| INT 20-2 | 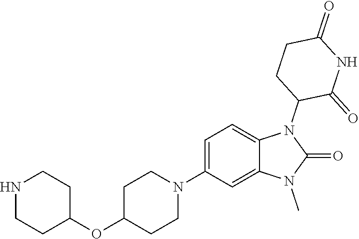 [SMILES: Cn1c(=O)n(C2CCC(=O)NC2=O)c2ccc(N3CCC(OC4CCNCC4)CC3)cc21] | 442.2 | tert-butyl 4-(piperidin-4- yloxy)piperidine-1-carboxylate in step 1 |

### Intermediates 21-1-21-2.

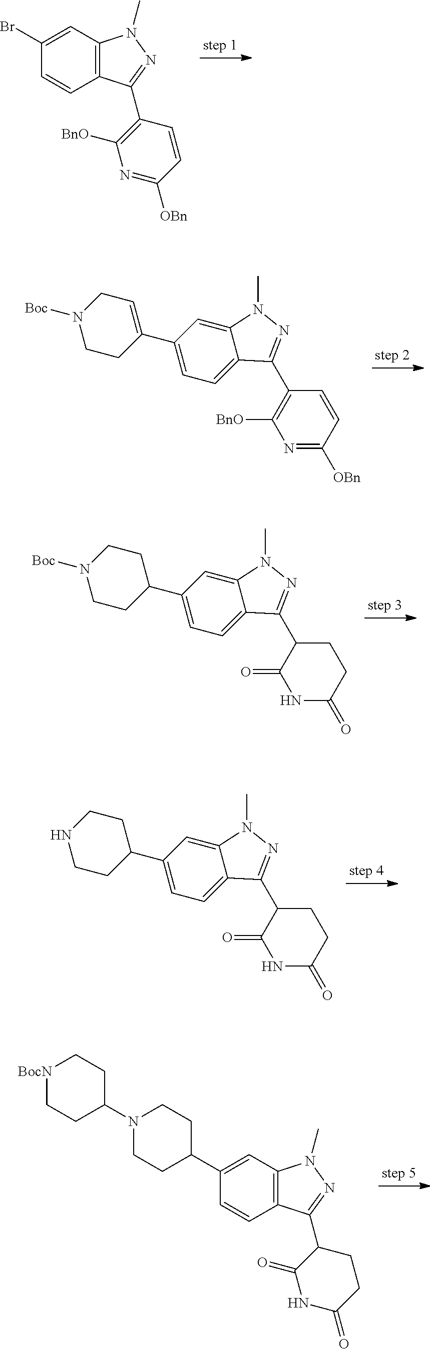 [SMILES: Cn1nc(-c2ccc(OCc3ccccc3)nc2OCc2ccccc2)c2ccc(Br)cc21 ; Cn1nc(-c2ccc(OCc3ccccc3)nc2OCc2ccccc2)c2ccc(C3=CCN(C(=O)OC(C)(C)C)CC3)cc21 ; Cn1nc(C2CCC(=O)NC2=O)c2ccc(C3CCN(C(=O)OC(C)(C)C)CC3)cc21 ; Cn1nc(C2CCC(=O)NC2=O)c2ccc(C3CCNCC3)cc21 ; Cn1nc(C2CCC(=O)NC2=O)c2ccc(C3CCN(C4CCN(C(=O)OC(C)(C)C)CC4)CC3)cc21]

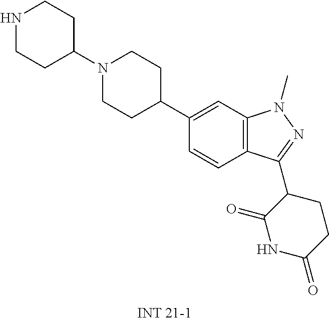 [SMILES: Cn1nc(C2CCC(=O)NC2=O)c2ccc(C3CCN(C4CCNCC4)CC3)cc21]

Step 1. tert-butyl 4-(3-(2,6-bis(benzyloxy)pyridin-3-yl)-1-methyl-1H-indazol-6-yl)-3,6-dihydropyridine-1(2H)-carboxylate. To a stirred solution of 3-(2,6-bis(benzyloxy)pyridin-3-yl)-6-bromo-1-methyl-1H-indazole (20 mmol) and tert-butyl 4-(4,4,5,5-tetramethyl-1,3,2-dioxaborolan-2-yl)-3,6-dihydropyridine-1(2H)-carboxylate (30 mmol) in a mixture of 1,4-dioxane and water (100 mL, 3:1) was added potassium carbonate (40 mmol) at room temperature. The reaction was degassed with nitrogen for 30 minutes. [1,1′-bis(diphenylphosphino)ferrocene]dichloropalladium(II) (2.0 mmol) was added to the reaction mixture and it was degassed with nitrogen for 10 minutes and heated to 100° C. for 12 hours. After completion, the reaction mixture was cooled to room temperature and filtered through a pad of Celite, washing with ethyl acetate. The filtrate was diluted with water and extracted with ethyl acetate. The combined organic fractions were washed with brine, dried over sodium sulfate, filtered, and was concentrated under reduced pressure to yield the title compound, which was used in the next step without purification. LCMS: 603.3.

Step 2: tert-butyl 4-(3-(2,6-dioxopiperidin-3-yl)-1-methyl-1H-indazol-6-yl)piperidine-1-carboxylate. To a stirred solution of tert-butyl 4-(3-(2,6-bis(benzyloxy)pyridin-3-yl)-1-methyl-1H-indazol-6-yl)-3,6-dihydropyridine-1(2H)-carboxylate (17.4 mmol) in ethyl acetate (105 mL) was added palladium on carbon (12 mmol, 10 wt % Pd) at room temperature. The reaction mixture was stirred in an autoclave under a hydrogen atmosphere (90 psi) for 12 hours at room temperature. After completion, the reaction mixture was cooled to room temperature and filtered through a pad of Celite, washing with ethyl acetate. The combined organic fractions were concentrated under reduced pressure to yield the title compound, which was triturated with diethyl ether and used in the next step.

Step 3: 3-(1-methyl-6-(piperidin-4-yl)-1H-indazol-3-yl)piperidine-2,6-dione. To a stirred solution of tert-butyl 4-(3-(2,6-dioxopiperidin-3-yl)-1-methyl-1H-indazol-6-yl)piperidine-1-carboxylate (11.7 mmol) in dichloromethane (36 mL) was added hydrochloric acid in 1,4-dioxane (11 mL, 4M) at 0° C. for 12 hours. After completion, the reaction mixture was concentrated under reduced pressure to yield the title compound, which was used in the next step without purification. LCMS: 327.3.

Step 4: tert-Butyl 4-(3-(2,6-dioxopiperidin-3-yl)-1-methyl-1H-indazol-6-yl)-[1,4′-bipiperidine]-1′-carboxylate. To a stirring suspension of 3-(1-methyl-6-(piperidin-4-yl)-1H-indazol-3-yl)piperidine-2,6-dione hydrochloride (0.77 mmol), tert-butyl 4-oxopiperidine-1-carboxylate (7.7 mmol), and sodium acetate (3.8 mmol) in methanol (4 mL) and N,N-dimethylformamide (1 mL) was added acetic acid (3.8 mmol) followed by sodium cyanoborohydride (7.7 mmol). The resulting mixture was heated to 50° C. After 3 hours, the reaction was cooled to room temperature and concentrated under reduced pressure. The residue was purified by flash column chromatography on silica gel (eluting with methanol in dichloromethane) to afford the title compound. LCMS: 510.0.

Step 5: 3-(6-([1,4′-bipiperidin]-4-yl)-1-methyl-1H-indazol-3-yl)piperidine-2,6-dione (INT 21-1). Trifluoracetic acid (9.1 mmol) was added to a stirring solution of tert-butyl 4-(3-(2,6-dioxopiperidin-3-yl)-1-methyl-1H-indazol-6-yl)-[1,4′-bipiperidine]-1′-carboxylate (0.46 mmol) in dichloromethane (4.0 mL) and the mixture was stirred at room temperature. After 30 minutes, a 4:1 mixture of dichloromethane:isopropanol and aqueous saturated sodium bicarbonate solution were added sequentially. The organic phase was collected and the aqueous phase was washed with 4:1 dichloromethane:isopropanol (2×). The combined organic layers were dried over anhydrous magnesium sulfate, filtered, and concentrated under reduced pressure to give the title compound without further purification. LCMS: 410.0.

The following intermediates were made in a similar fashion to INT 21-1 and are shown below in Table 2O. To prepare the below intermediates, different reagents/starting materials were used than previously described in the steps toward INT 21-1 and are noted in the last column of Table 2O—“Changes to Procedure: Different Reagents/Starting Materials”. A person of ordinary skill in the art will readily recognize which reagents/starting materials used for the synthesis of INT 21-1 were replaced with the different reagents/starting materials noted below.

TABLE 2O
Intermediates

| | | | Changes to Procedure: Different |
|---|---|---|---|
| Intermediate | Structure | LCMS | Reagents/Starting Materials |
| INT 21-2 | 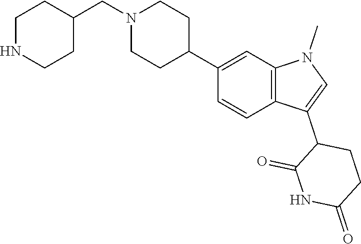 [SMILES: Cn1cc(C2CCC(=O)NC2=O)c2ccc(C3CCN(CC4CCNCC4)CC3)cc21] | 424.3 | tert-butyl 4-formylpiperidine-1- carboxylate in step 5. Hydrochloric acid in 1,4-dioxane in step 6. |

### Intermediate 22-1.

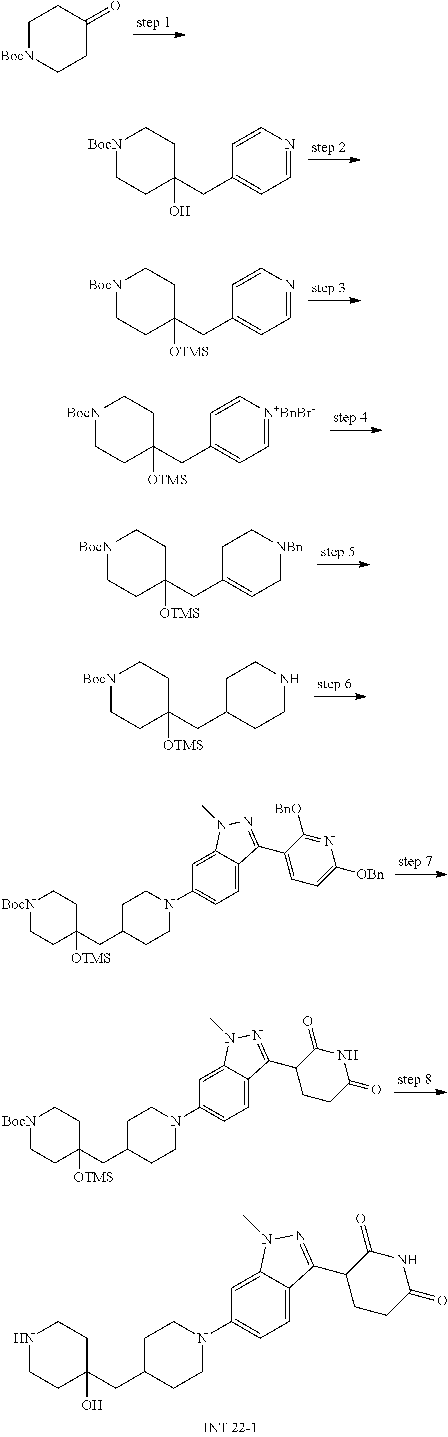

Step 1: tert-butyl 4-hydroxy-4-(pyridin-4-ylmethyl)piperidine-1-carboxylate. To a solution of 4-methylpyridine (21.5 mmol) in tetrahydrofuran (20 mL) at −78° C. was added a solution of n-butyllithium (2.5M in hexanes, 25.8 mmol). The reaction mixture was vigorously stirred at −78° C. for 1 hour. A solution of tert-butyl 4-oxopiperidine-1-carboxylate (21.5 mmol) in tetrahydrofuran (10 mL) was slowly added into the reaction mixture at −78° C. The solution was stirred and slowly warmed to room temperature over 16 hours. Upon completion, the solution was quenched with water (10 mL) and extracted with ethyl acetate (3×20 mL), then concentrated under reduced pressure. The residue was purified by flash column chromatography on silica gel (0% to 100% ethyl acetate in hexanes) to afford the title compound. LCMS: 293.3.

Step 2: tert-butyl 4-(pyridin-4-ylmethyl)-4-((trimethylsilyl)oxy)piperidine-1-carboxylate. A solution of tert-butyl 4-hydroxy-4-(4-pyridylmethyl)piperidine-1-carboxylate (13.7 mmol) and imidazole (68.4 mmol) in dichloromethane (50 mL) was slowly added chlorotrimethylsilane (27.4 mmol) at 0° C. The solution was stirred and slowly warmed to room temperature over 16 hours. Upon completion, the solution was quenched with water (10 mL) and extracted with dichloromethane (3×50 mL), then concentrated under reduced pressure. The residue was purified by flash column chromatography on silica gel (0% to 30% ethyl acetate in hexanes) to afford the title compound. LCMS: 365.3.

Step 3: 1-benzyl-4-((1-(tert-butoxycarbonyl)-4-((trimethylsilyl)oxy)piperidin-4-yl)methyl)pyridin-1-ium bromide. To a solution of tert-butyl 4-(4-pyridylmethyl)-4-trimethylsilyloxy-piperidine-1-carboxylate (13.7 mmol) in toluene (50 mL) was added benzyl bromide (16.5 mmol) at room temperature. The reaction mixture was heated at 80° C. overnight. Upon completion, the solution was concentrated and formed a solid, which was then filtered and washed with hexanes (4×50 mL). The solid was collected and used for the next step. LCMS: 455.3.

Step 4: tert-butyl 4-((1-benzyl-1,2,3,6-tetrahydropyridin-4-yl)methyl)-4-((trimethylsilyl)oxy)piperidine-1-carboxylate. To a solution of 1-benzyl-4-((1-(tert-butoxycarbonyl)-4-((trimethylsilyl)oxy)piperidin-4-yl)methyl)pyridin-1-ium bromide (13.2 mmol) in ethanol (100 mL) was added sodium borohydride (79 mmol) at 0° C. portionwise. After stirring at 0° C. for 3 hours, the reaction was concentrated to give a yellow residue which was then dissolved in a mixture of dichloromethane/water (v:v 1:1, 200 mL), and extracted by dichloromethane (3×50 mL). The residue was purified by flash column chromatography on silica gel (0% to 100% ethyl acetate in hexanes) to afford the title compound. LCMS: 459.4.

Step 5: tert-butyl 4-(piperidin-4-ylmethyl)-4-((trimethylsilyl)oxy)piperidine-1-carboxylate. A mixture of palladium hydroxide on carbon (4 mmol) and benzyl 4-((1-(2-(2,6-dioxopiperidin-3-yl)-1-oxoisoindolin-5-yl)-4-methylpiperidin-4-yl)methyl)piperazine-1-carboxylate (4 mmol) in ethanol (20 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×20 mL), concentrated under reduced pressure and used for the next step. LCMS: 371.4.

Step 6: tert-butyl 4-((1-(3-(2,6-bis(benzyloxy)pyridin-3-yl)-1-methyl-1H-indazol-6-yl)piperidin-4-yl)methyl)-4-((trimethylsilyl)oxy)piperidine-1-carboxylate. A vigorously stirred solution of tert-butyl 4-(piperidin-4-ylmethyl)-4-((trimethylsilyl)oxy)piperidine-1-carboxylate (1.2 mmol), 6-bromo-3-(2,6-dibenzyloxy-3-pyridyl)-1-methyl-indazole (1.0 mmol), tris(dibenzylideneacetone)dipalladium(0) (0.02 mmol), 2-dicyclohexylphosphino-2′,4′,6′-triisopropylbiphenyl (0.03 mmol), and cesium carbonate (3.0 mmol) in 1,4-dioxane (7 mL) was heated under argon at 100° C. for 16 hours. The reaction mixture was filtered through a short pad of Celite and washed with dichloromethane (3×10 mL), then concentrated under reduced pressure. The residue was purified by flash column chromatography on silica gel (100% ethyl acetate) to afford the title compound. LCMS: 790.4.

Step 7: tert-butyl 4-((1-(3-(2,6-dioxopiperidin-3-yl)-1-methyl-1H-indazol-6-yl)piperidin-4-yl)methyl)-4-((trimethylsilyl)oxy)piperidine-1-carboxylate. A mixture of palladium hydroxide on carbon (3.28 mmol) and tert-butyl 4-((1-(3-(2,6-bis(benzyloxy)pyridin-3-yl)-1-methyl-1H-indazol-6-yl)piperidin-4-yl)methyl)-4-((trimethylsilyl)oxy)piperidine-1-carboxylate (0.58 mmol) in tetrahydrofuran (5 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×10 mL), then concentrated under reduced pressure. The residue was purified by flash column chromatography on silica gel (0% to 50% ethyl acetate in hexanes) to afford the title compound. LCMS: 612.4.

Step 8: 3-(6-(4-((4-hydroxypiperidin-4-yl)methyl)piperidin-1-yl)-1-methyl-1H-indazol-3-yl)piperidine-2,6-dione (INT 22-1). Trifluoroacetic acid (0.5 mL) was added into a solution of tert-butyl 4-((1-(3-(2,6-dioxopiperidin-3-yl)-1-methyl-1H-indazol-6-yl)piperidin-4-yl)methyl)piperazine-1-carboxylate (0.58 mmol) in dichloromethane (5 mL) at room temperature. The resulting solution was stirred at room temperature for 2 hours. Upon completion, the solution was concentrated under reduced pressure and used for the next step without further purification. LCMS: 440.3.

### Intermediate 23-1.

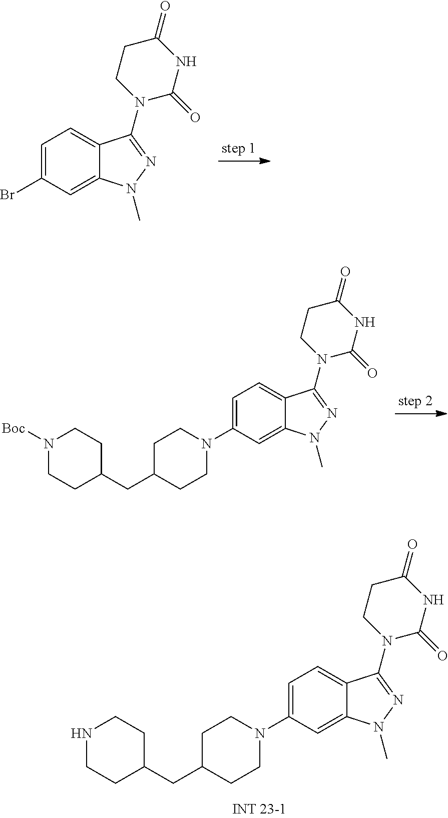 [SMILES: Cn1nc(N2CCC(=O)NC2=O)c2ccc(Br)cc21 ; Cn1nc(N2CCC(=O)NC2=O)c2ccc(N3CCC(CC4CCN(C(=O)OC(C)(C)C)CC4)CC3)cc21 ; Cn1nc(N2CCC(=O)NC2=O)c2ccc(N3CCC(CC4CCNCC4)CC3)cc21]

Step 1: tert-butyl 4-((1-(3-(2,4-dioxotetrahydropyrimidin-1(2H)-yl)-1-methyl-1H-indazol-6-yl)piperidin-4-yl)methyl)piperidine-1-carboxylate. To a mixture of 1-(6-bromo-1-methyl-1H-indazol-3-yl)dihydropyrimidine-2,4(1H,3H)-dione (3.1 mmol) and tert-butyl 4-(4-piperidylmethyl)piperidine-1-carboxylate (4.6 mmol) in dimethyl sulfoxide (6 mL) was added cesium carbonate (6.1 mmol) and dichloro[1,3-bis(2,6-di-3-pentylphenyl)imidazol-2-ylidene](3-chloropyridyl)palladium(II) (0.3 mmol). The mixture was stirred at 110° C. for 2 hours under a nitrogen atmosphere. The reaction mixture was then cooled to room temperature, filtered and concentrated under reduced pressure to give a residue. The residue was purified by prep-HPLC (20% to 50% acetonitrile/water with 0.04% hydrochloric acid), and fractions containing the desired product were pooled and lyophilized to yield the title compound. LCMS: 525.4.

Step 2: 1-(1-methyl-6-(4-(piperidin-4-ylmethyl)piperidin-1-yl)-1H-indazol-3-yl)dihydropyrimidine-2,4(1H,3H)-dione (INT 23-1). To a mixture of tert-butyl 4-((1-(3-(2,4-dioxotetrahydropyrimidin-1(2H)-yl)-1-methyl-1H-indazol-6-yl)piperidin-4-yl)methyl)piperidine-1-carboxylate (0.57 mmol) in ethyl acetate (3 mL) was added hydrochloric acid in dioxane (1 mL, 4 M). The mixture was stirred at room temperature for 1 hour. The reaction mixture was concentrated under reduced pressure to give a residue, which was purified by triturating with ethyl acetate to afford the title compound. LCMS: 425.2.

### Intermediates 24-1-24-2.

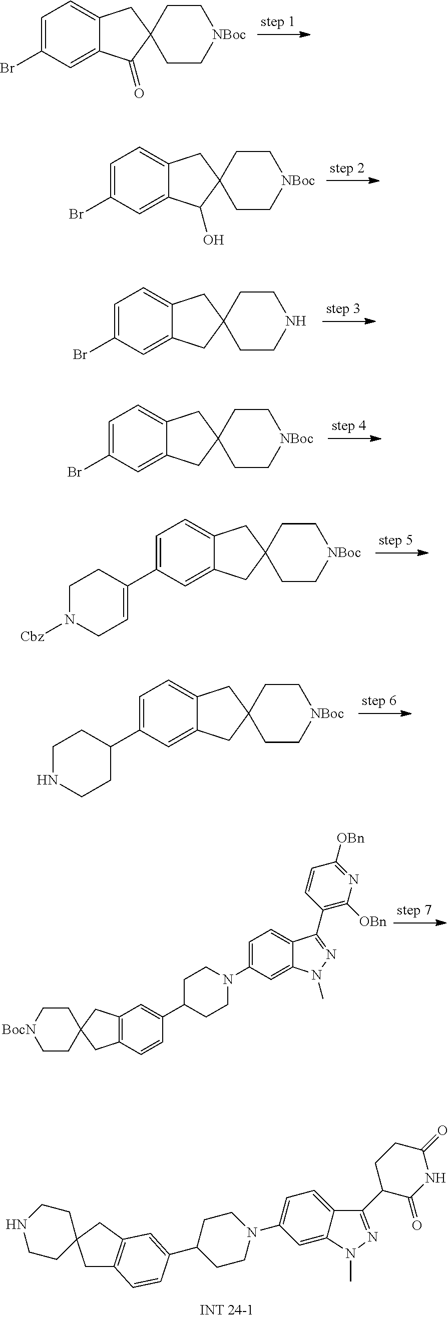

Step 1: tert-butyl 6-bromo-1-hydroxy-1,3-dihydrospiro[indene-2,4′-piperidine]-1′-carboxylate. To a solution of tert-butyl 6-bromo-1-oxo-spiro[indane-2,4′-piperidine]-1′-carboxylate (2.6 mmol) in methanol (10 mL) was added sodium borohydride (3.9 mmol) at 0° C. The reaction mixture was allowed to warm to room temperature and stirred for 1 hour. After cooling to 0° C., 1 M hydrochloric acid (10 mL) was added, and the aqueous layer was extracted with ethyl acetate (3×30 mL). The organic layers were combined, washed with brine (50 mL), dried over anhydrous sodium sulfate, filtered, concentrated, and used in the subsequent reaction without further purification. LCMS: 405.9.

Step 2: 5-bromo-1,3-dihydrospiro[indene-2,4′-piperidine]. To a solution of tert-butyl 6-bromo-1-hydroxy-1,3-dihydrospiro[indene-2,4′-piperidine]-1′-carboxylate (2.6 mmol) in trifluoroacetic acid (2 mL) was added triethylsilane (26.2 mmol) dropwise at 0° C. The reaction mixture was stirred overnight at 40° C., concentrated to dryness under reduced pressure and dried further under high vacuum for two days. The crude product was then used in the subsequent reaction without further purification. LCMS: 266.0.

Step 3: tert-butyl 5-bromo-1,3-dihydrospiro[indene-2,4′-piperidine]-1′-carboxylate. To a solution of 5-bromo-1,3-dihydrospiro[indene-2,4′-piperidine] (3.9 mmol) in methanol (10 mL) at 0° C. was added 2 M sodium hydroxide (4 mL) followed by di-tert-butyl dicarbonate (4.7 mmol). The ice bath was removed, and the reaction mixture was stirred overnight at room temperature. 1 M hydrochloric acid (15 mL) was then added at 0° C. and extracted with ethyl acetate (3×20 mL). The organic layers were combined, washed with brine (30 mL), dried over anhydrous sodium sulfate, filtered, concentrated and purified by flash column chromatography eluting with ethyl acetate/hexanes to afford the title compound. LCMS: 309.9.

Step 4: tert-butyl 5-(1-((benzyloxy)carbonyl)-1,2,3,6-tetrahydropyridin-4-yl)-1,3-dihydrospiro[indene-2,4′-piperidine]-1′-carboxylate. To a vial was added tert-butyl 5-bromo-1,3-dihydrospiro[indene-2,4′-piperidine]-1′-carboxylate (0.5 mmol), benzyl 4-(4,4,5,5-tetramethyl-1,3,2-dioxaborolan-2-yl)-3,6-dihydro-2H-pyridine-1-carboxylate (0.7 mmol), [1,1′-bis(diphenylphosphino)ferrocene]dichloropalladium(II) (0.05 mmol), cesium carbonate (1.6 mmol), 1,4-dioxane (2 mL) and water (0.2 mL). The mixture was degassed with argon for 5 minutes. The vial was sealed and the reaction mixture was stirred at 90° C. overnight. The reaction mixture was then allowed to cool to room temperature, filtered, concentrated under reduced pressure, and purified by flash column chromatography eluting with ethyl acetate/hexanes to afford the title compound. LCMS: 447.0.

Step 5: tert-butyl 5-(piperidin-4-yl)-1,3-dihydrospiro[indene-2,4′-piperidine]-1′-carboxylate. To a solution of tert-butyl 5-(1-((benzyloxy)carbonyl)-1,2,3,6-tetrahydropyridin-4-yl)-1,3-dihydrospiro[indene-2,4′-piperidine]-1′-carboxylate (0.4 mmol) in ethanol (5 mL) was added palladium on carbon (0.04 mmol of 10 wt. % Pd). The resulting reaction mixture was stirred overnight under a hydrogen atmosphere (1 atm), filtered, concentrated under reduced pressure, and used in the subsequent reaction without further purification. LCMS: 371.1.

Step 6: tert-butyl 5-(1-(3-(2,6-bis(benzyloxy)pyridin-3-yl)-1-methyl-1H-indazol-6-yl)piperidin-4-yl)-1,3-dihydrospiro[indene-2,4′-piperidine]-1′-carboxylate. A mixture of 3-(2,6-bis(benzyloxy)pyridin-3-yl)-6-bromo-1-methyl-1H-indazole (0.6 mmol), tert-butyl 5-(piperidin-4-yl)-1,3-dihydrospiro[indene-2,4′-piperidine]-1′-carboxylate (0.7 mmol), 2-dicyclohexylphosphino-2′,4′,6′-triisopropylbiphenyl (0.2 mmol), cesium carbonate (1.2 mmol), and palladium acetate (0.1 mmol) in 1,4-dioxane (10 mL) was degassed with argon for 1 minute. The reaction mixture was stirred at 100° C. for 16 hours under an argon atmosphere. The reaction mixture was filtered through Celite and the filtrate was concentrated under reduced pressure. The residue was purified by flash column chromatography eluting with ethyl acetate/hexanes to afford the title compound. LCMS: 790.0.

Step 7: 3-(6-(4-(1,3-dihydrospiro[indene-2,4′-piperidin]-5-yl)piperidin-1-yl)-1-methyl-1H-indazol-3-yl)piperidine-2,6-dione (INT 24-1). To a solution of tert-butyl 5-(1-(3-(2,6-bis(benzyloxy)pyridin-3-yl)-1-methyl-1H-indazol-6-yl)piperidin-4-yl)-1,3-dihydrospiro[indene-2,4′-piperidine]-1′-carboxylate (0.4 mmol) in tetrahydrofuran (6 mL) was added palladium hydroxide on carbon (2.0 mmol, 20 wt % Pd). The reaction mixture was evacuated and backfilled with hydrogen three times and stirred at room temperature for 16 hours. The reaction mixture was filtered through Celite and the filtrate was concentrated under reduced pressure. The residue was dissolved in dichloromethane (3 mL) and trifluoroacetic acid (9.9 mmol) was added at room temperature and the reaction mixture stirred for 1 hour. The reaction mixture was concentrated and purified by RP-HPLC (0 to 90%, 0.1% trifluoroacetic acid in acetonitrile/0.1% trifluoroacetic acid in water). Fractions containing the product were pooled and lyophilized to afford the title compound. LCMS: 512.1.

The following intermediates were made in a similar fashion to INT 24-1 and are shown below in Table 2P. To prepare the below intermediates, different reagents/starting materials were used than previously described in the steps toward INT 24-1 and are noted in the last column of Table 2P—“Changes to Procedure: Different Reagents/Starting Materials”. A person of ordinary skill in the art will readily recognize which reagents/starting materials used for the synthesis of INT 24-1 were replaced with the different reagents/starting materials noted below.

TABLE 2O
Intermediates

| | | | Changes to Procedure: Different |
|---|---|---|---|
| Intermediate | Structure | LCMS | Reagents/Starting Materials |
| INT 24-2 | 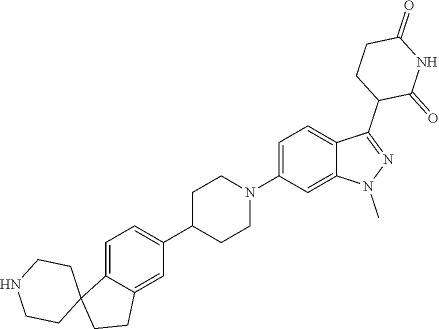 [SMILES: Cn1nc(C2CCC(=O)NC2=O)c2ccc(N3CCC(c4ccc5c(c4)CCC54CCNCC4)CC3)cc21] | 512.1 | tert-butyl 5-bromo-3-oxo-2,3- dihydrospiro[indene-1,4′-piperidine]-1′- carboxylate in step 6. |

### Intermediate 24-3.

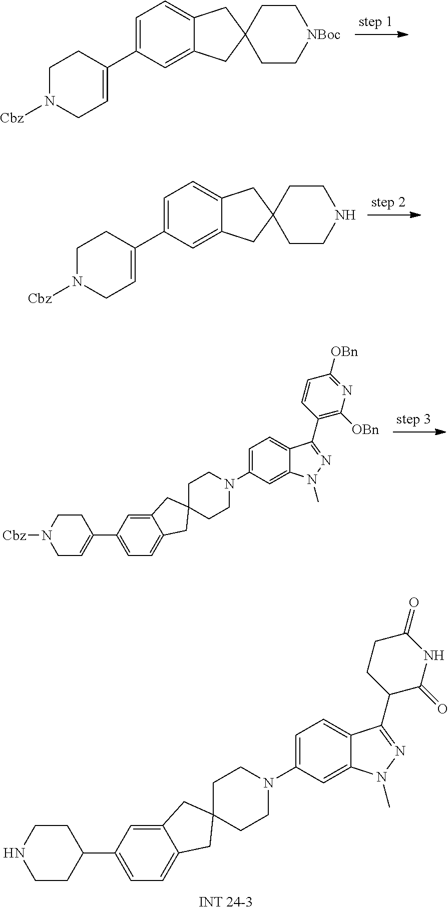 [SMILES: CC(C)(C)OC(=O)N1CCC2(CC1)Cc1ccc(C3=CCN(C(=O)OCc4ccccc4)CC3)cc1C2 ; O=C(OCc1ccccc1)N1CC=C(c2ccc3c(c2)CC2(CCNCC2)C3)CC1 ; Cn1nc(-c2ccc(OCc3ccccc3)nc2OCc2ccccc2)c2ccc(N3CCC4(CC3)Cc3ccc(C5=CCN(C(=O)OCc6ccccc6)CC5)cc3C4)cc21 ; Cn1nc(C2CCC(=O)NC2=O)c2ccc(N3CCC4(CC3)Cc3ccc(C5CCNCC5)cc3C4)cc21]

Step 1: Benzyl 4-(1,3-dihydrospiro[indene-2,4′-piperidin]-5-yl)-3,6-dihydropyridine-1(2H)-carboxylate. To a stirred solution of tert-butyl 5-(1-((benzyloxy)carbonyl)-1,2,3,6-tetrahydropyridin-4-yl)-1,3-dihydrospiro[indene-2,4′-piperidine]-1′-carboxylate (0.80 mmol) in dichloromethane (3.0 mL) was added trifluoroacetic acid (0.30 mL) at room temperature. The mixture was stirred for 1 hour. After completion, the reaction mixture was concentrated under reduced pressure to afford a residue, which was purified by trituration with diethyl ether to yield the title compound. LCMS: 403.1.

Step 2: Benzyl 4-(1′-(3-(2,6-dioxo-1-((2-(trimethylsilyl)ethoxy)methyl)piperidin-3-yl)-1-methyl-1H-indazol-6-yl)-1,3-dihydrospiro[indene-2,4′-piperidin]-5-yl)-3,6-dihydropyridine-1(2H)-carboxylate. A vigorously stirred mixture of 3-(6-bromo-1-methyl-indazol-3-yl)-1-(2-trimethylsilylethoxymethyl)piperidine-2,6-dione (0.70 mmol), benzyl 4-(1,3-dihydrospiro[indene-2,4′-piperidin]-5-yl)-3,6-dihydropyridine-1(2H)-carboxylate (0.80 mmol), X-Phos (0.20 mmol), cesium carbonate (2.0 mmol), and palladium(II) acetate (0.10 mmol) in 1,4-dioxane (6.0 mL) was heated to 90° C. and stirred under an argon atmosphere for 16 hours. Upon completion, the reaction mixture was cooled and filtered through a pad of Celite. The filtrate was concentrated under reduced pressure and the resulting residue was purified by flash column chromatography (ethyl acetate/hexanes) to afford the title compound. LCMS: 774.0.

Step 7: 3-(1-methyl-6-(5-(piperidin-4-yl)-1,3-dihydrospiro[indene-2,4′-piperidin]-1′-yl)-1H-indazol-3-yl)piperidine-2,6-dione (INT 24-3). To a solution of benzyl 4-(1′-(3-(2,6-dioxo-1-((2-(trimethylsilyl)ethoxy)methyl)piperidin-3-yl)-1-methyl-1H-indazol-6-yl)-1,3-dihydrospiro[indene-2,4′-piperidin]-5-yl)-3,6-dihydropyridine-1(2H)-carboxylate (0.10 mmol) in ethanol (5.0 mL) was added palladium on carbon (10 wt % Pd, 0.05 mmol). The reaction mixture was evacuated and backfilled with hydrogen three times, then stirred at room temperature for 16 hours. The mixture was filtered through a pad of Celite, and the filtrate was concentrated under reduced pressure. The residue was dissolved in dichloromethane (3.0 mL) and treated with trifluoroacetic acid (0.50 mL) at room temperature. After stirring for 1 hour, the reaction mixture was concentrated and purified by reverse-phase HPLC (0-90% gradient of 0.1% trifluoroacetic acid in acetonitrile/water). Fractions containing the product were pooled and lyophilized to afford the title compound. LCMS: 512.1.

### Intermediate 25-1.

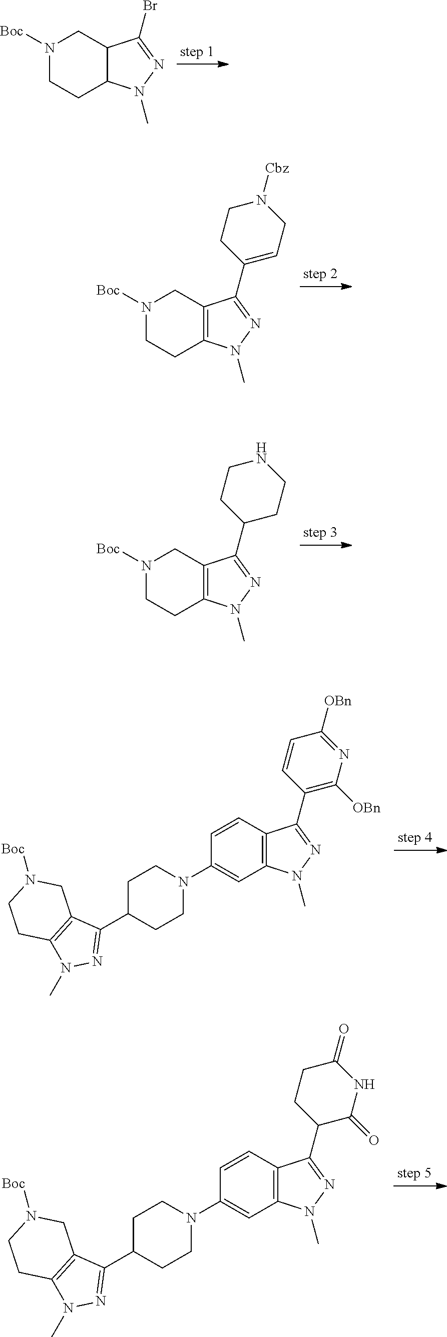 [SMILES: CN1N=C(Br)C2CN(C(=O)OC(C)(C)C)CCC21 ; Cn1nc(C2=CCN(C(=O)OCc3ccccc3)CC2)c2c1CCN(C(=O)OC(C)(C)C)C2 ; Cn1nc(C2CCNCC2)c2c1CCN(C(=O)OC(C)(C)C)C2 ; Cn1nc(C2CCN(c3ccc4c(-c5ccc(OCc6ccccc6)nc5OCc5ccccc5)nn(C)c4c3)CC2)c2c1CCN(C(=O)OC(C)(C)C)C2 ; Cn1nc(C2CCN(c3ccc4c(C5CCC(=O)NC5=O)nn(C)c4c3)CC2)c2c1CCN(C(=O)OC(C)(C)C)C2]

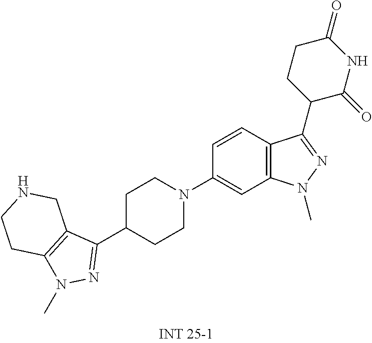 [SMILES: Cn1nc(C2CCN(c3ccc4c(C5CCC(=O)NC5=O)nn(C)c4c3)CC2)c2c1CCNC2]

Step 1: tert-butyl 3-(1-benzyloxycarbonyl-3,6-dihydro-2H-pyridin-4-yl)-1-methyl-6,7-dihydro-4H-pyrazolo[4,3-c]pyridine-5-carboxylate. To a solution of tert-butyl 3-bromo-1-methyl-6,7-dihydro-4H-pyrazolo[4,3-c]pyridine-5-carboxylate (1.6 mmol) in 1,4-dioxane (6 mL) was added benzyl 4-(4,4,5,5-tetramethyl-1,3,2-dioxaborolan-2-yl)-3,6-dihydro-2H-pyridine-1-carboxylate (1.7 mmol), [1,1′-bis(diphenylphosphino)ferrocene]dichloropalladium(II) (0.1 mmol), cesium carbonate (4.7 mmol), and water (0.8 mL). The resulting reaction mixture was degassed with argon for 30 seconds and subsequently stirred at 80° C. for 16 hours under an argon atmosphere. The reaction mixture was then filtered through Celite and concentrated under reduced pressure. The residue was purified by flash column chromatography on silica gel eluting with ethyl acetate/hexanes to afford the title compound. LCMS: 453.0.

Step 2: tert-butyl 1-methyl-3-(4-piperidyl)-6,7-dihydro-4H-pyrazolo[4,3-c]pyridine-5-carboxylate. To a solution of tert-butyl 3-(1-benzyloxycarbonyl-3,6-dihydro-2H-pyridin-4-yl)-1-methyl-6,7-dihydro-4H-pyrazolo[4,3-c]pyridine-5-carboxylate (1.1 mmol) in tetrahydrofuran (16 mL) was added palladium hydroxide on carbon (1.1 mmol, 20% wt. Pd). The reaction mixture was then evacuated and backfilled with hydrogen three times and stirred at room temperature for 16 hours. The reaction mixture was then filtered through Celite and concentrated under reduced pressure. The residue was used without the need for further purification. LCMS: 321.1.

Step 3: tert-butyl 3-[1-[3-(2,6-dibenzyloxy-3-pyridyl)-1-methyl-indazol-6-yl]-4-piperidyl]-1-methyl-6,7-dihydro-4H-pyrazolo[4,3-c]pyridine-5-carboxylate. A mixture of 3-(2,6-bis(benzyloxy)pyridin-3-yl)-6-bromo-1-methyl-1H-indazole (0.6 mmol), tert-butyl 1-methyl-3-(4-piperidyl)-6,7-dihydro-4H-pyrazolo[4,3-c]pyridine-5-carboxylate (0.7 mmol), 2-dicyclohexylphosphino-2′,4′,6′-triisopropylbiphenyl (0.2 mmol), palladium acetate (0.1 mmol), and cesium carbonate (1.2 mmol) in 1,4-dioxane (14 mL) was degassed with argon for 30 seconds. The reaction mixture was stirred at 100° C. for 16 hours under an argon atmosphere. The reaction mixture was filtered through Celite and the filtrate was concentrated under reduced pressure. The residue was purified by flash column chromatography eluting with ethyl acetate/hexanes to afford the title compound. LCMS: 740.0.

Step 4: tert-butyl 3-[1-[3-(2,6-dioxo-3-piperidyl)-1-methyl-indazol-6-yl]-4-piperidyl]-1-methyl-6,7-dihydro-4H-pyrazolo[4,3-c]pyridine-5-carboxylate. To a solution of tert-butyl 3-[1-[3-(2,6-dibenzyloxy-3-pyridyl)-1-methyl-indazol-6-yl]-4-piperidyl]-1-methyl-6,7-dihydro-4H-pyrazolo[4,3-c]pyridine-5-carboxylate (0.3 mmol) in tetrahydrofuran (5 mL) was added palladium hydroxide on carbon (0.3 mmol, 20% wt. Pd). The reaction mixture was then evacuated and backfilled with hydrogen three times and stirred at room temperature for 16 hours. The reaction mixture was then filtered through Celite and concentrated under reduced pressure. The residue was used without the need for further purification. LCMS: 562.0.

Step 5: 3-[1-methyl-6-[4-(1-methyl-4,5,6,7-tetrahydropyrazolo[4,3-c]pyridin-3-yl)-1-piperidyl]indazol-3-yl]piperidine-2,6-dione (INT 25-1). To a solution of tert-butyl 3-[1-[3-(2,6-dioxo-3-piperidyl)-1-methyl-indazol-6-yl]-4-piperidyl]-1-methyl-6,7-dihydro-4H-pyrazolo[4,3-c]pyridine-5-carboxylate (0.3 mmol) in dichloromethane (5 mL) was added trifluoroacetic acid (7.5 mmol). The reaction mixture was stirred at room temperature for 1 hour. The reaction mixture was concentrated and purified by RP-HPLC (0 to 90%, 0.1% trifluoroacetic acid in acetonitrile/0.1% trifluoroacetic acid in water). Fractions containing the product were pooled and lyophilized to afford the title compound. LCMS: 462.1.

### Intermediate 26-1.

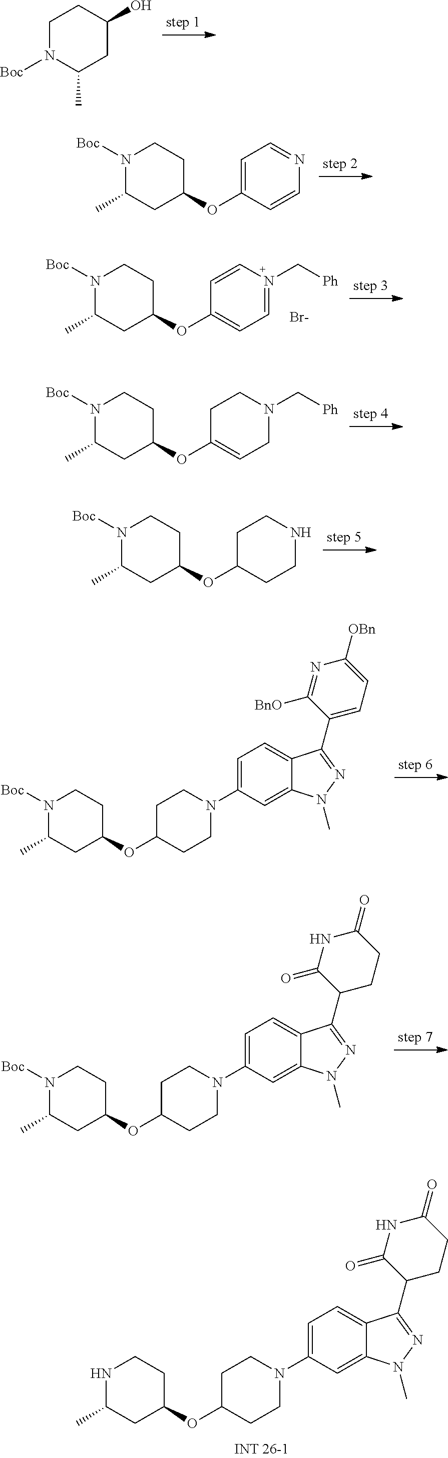

Step 1: tert-butyl (2S,4R)-2-methyl-4-(pyridin-4-yloxy)piperidine-1-carboxylate. To a solution of tert-butyl (2S,4R)-4-hydroxy-2-methylpiperidine-1-carboxylate (22.8 mmol) in dimethyl sulfoxide (65 mL) was added potassium tert-butoxide (68.4 mmol) in one portion. The reaction mixture was stirred for 5 minutes, then 4-chloropyridine hydrochloride (22.8 mmol) was added in one portion. The reaction mixture was stirred at 60° C. for 4 hours. The resulting mixture was cooled to room temperature and diluted with water (100 mL) and diethyl ether (100 mL). The organic layer was removed, and the aqueous layer was extracted with diethyl ether (3×100 mL). The organic layers were combined, washed with brine (30 mL), dried over anhydrous sodium sulfate, filtered, and concentrated under reduced pressure. The residue was purified by flash column chromatography on silica gel eluting with ethyl acetate/dichloromethane to afford the title compound. LCMS: 293.2.

Step 2: 1-benzyl-4-(((2S,4R)-1-(tert-butoxycarbonyl)-2-methylpiperidin-4-yl)oxy)pyridin-1-ium bromide. To a solution of tert-butyl (2S,4R)-2-methyl-4-(pyridin-4-yloxy)piperidine-1-carboxylate (19.2 mmol) in dichloromethane (55 mL) was added benzyl bromide (28.8 mmol) dropwise at room temperature. The resulting mixture was stirred at room temperature overnight. The solution was concentrated under reduced pressure. The resulting mixture was suspended in hexane and sonicated for 30 min. The product was filtered, washed with hexanes, and dried under reduced pressure overnight to give the title compound. LCMS: 383.3.

Step 3: tert-butyl (2S,4R)-4-((1-benzyl-1,2,3,6-tetrahydropyridin-4-yl)oxy)-2-methylpiperidine-1-carboxylate. To a solution of 1-benzyl-4-(((2S,4R)-1-(tert-butoxycarbonyl)-2-methylpiperidin-4-yl)oxy)pyridin-1-ium bromide (19.2 mmol) in methanol (75 mL) at 0° C. was added sodium borohydride (76.8 mmol) in small portions. After the gas evolution ceased, the ice bath was removed, and the resulting solution was stirred at room temperature for 1 h. The solution was then concentrated, diluted with water (100 mL) and extracted with dichloromethane (3×100 mL). The organic layers were combined, washed with brine (30 mL), dried over anhydrous sodium sulfate, filtered, and concentrated under reduced pressure. The residue was purified by flash column chromatography on silica gel eluting with ethyl acetate/hexanes to afford the title compound. LCMS: 387.3.

Step 4: tert-butyl (2S,4R)-2-methyl-4-(piperidin-4-yloxy)piperidine-1-carboxylate. To a solution of (2S,4R)-4-((1-benzyl-1,2,3,6-tetrahydropyridin-4-yl)oxy)-2-methylpiperidine-1-carboxylate (12.7 mmol) in ethanol (63.6 mL) was added palladium hydroxide on carbon (2.45 g, 20 wt. % Pd) and stirred at room temperature overnight under an atmosphere of hydrogen (1 atm). The resulting mixture was then filtered and concentrated to dryness under reduced pressure to provide the title compound. LCMS: 299.3.

Step 5: tert-butyl (2S,4R)-4-((1-(3-(2,6-bis(benzyloxy)pyridin-3-yl)-1-methyl-1H-indazol-6-yl)piperidin-4-yl)oxy)-2-methylpiperidine-1-carboxylate. To a vial were added 3-(2,6-bis(benzyloxy)pyridin-3-yl)-6-bromo-1-methyl-1H-indazole (0.5 mmol), tert-butyl (2S,4R)-2-methyl-4-(piperidin-4-yloxy)piperidine-1-carboxylate (0.55 mmol), 2-dicyclohexylphosphino-2′,4′,6′-triisopropylbiphenyl (0.015 mmol), cesium carbonate (1.5 mmol), tris(dibenzylideneacetone)dipalladium(0) (0.01 mmol) and 1,4-dioxane (5 mL). The resulting mixture was degassed with argon for 20 minutes, then stirred at 100° C. overnight. The reaction mixture was then filtered, and the filtrate was concentrated under reduced pressure. The residue was purified by flash column chromatography on silica gel by eluting with ethyl acetate/hexanes to afford the title compound. LCMS: 718.5.

Step 6: tert-butyl (2S,4R)-4-((1-(3-(2,6-dioxopiperidin-3-yl)-1-methyl-1H-indazol-6-yl)piperidin-4-yl)oxy)-2-methylpiperidine-1-carboxylate. To a solution of tert-butyl (2S,4R)-4-((1-(3-(2,6-bis(benzyloxy)pyridin-3-yl)-1-methyl-1H-indazol-6-yl)piperidin-4-yl)oxy)-2-methylpiperidine-1-carboxylate (0.27 mmol) in ethanol (5.1 mL) was added palladium hydroxide on carbon (192 mg, 20 wt. % Pd) and stirred at room temperature overnight under an atmosphere of hydrogen (1 atm). The resulting mixture was then filtered and concentrated to dryness under reduced pressure to provide the title compound. LCMS: 540.4.

Step 7: 3-(1-methyl-6-(4-(((2S,4R)-2-methylpiperidin-4-yl)oxy)piperidin-1-yl)-1H-indazol-3-yl)piperidine-2,6-dione (INT 26-1). Trifluoroacetic acid (0.2 mL) was added to a stirred solution of tert-butyl (2S,4R)-4-((1-(3-(2,6-dioxopiperidin-3-yl)-1-methyl-1H-indazol-6-yl)piperidin-4-yl)oxy)-2-methylpiperidine-1-carboxylate (0.27 mmol) in dichloromethane (3 mL) at room temperature. After stirring at room temperature for 3 hours, the reaction mixture was concentrated under reduced pressure to yield the title compound which was used without further purification. LCMS: 440.3.

### Intermediate 27-1.

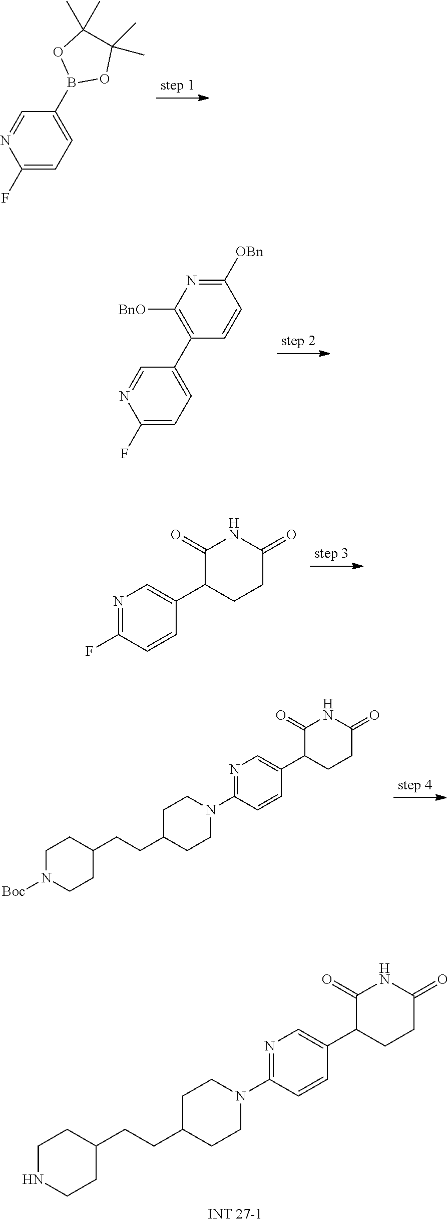 [SMILES: CC1(C)OB(c2ccc(F)nc2)OC1(C)C ; Fc1ccc(-c2ccc(OCc3ccccc3)nc2OCc2ccccc2)cn1 ; O=C1CCC(c2ccc(F)nc2)C(=O)N1 ; CC(C)(C)OC(=O)N1CCC(CCC2CCN(c3ccc(C4CCC(=O)NC4=O)cn3)CC2)CC1 ; O=C1CCC(c2ccc(N3CCC(CCC4CCNCC4)CC3)nc2)C(=O)N1]

Step 1: 2,6-bis(benzyloxy)-6′-fluoro-3,3′-bipyridine. To a solution of 2,6-dibenzyloxy-3-bromo-pyridine (203 mmol) in dioxane (800 mL) was added 2-fluoro-5-(4,4,5,5-tetramethyl-1,3,2-dioxaborolan-2-yl)pyridine (243.0 mmol), [1,1′-bis(diphenylphosphino)ferrocene]dichloropalladium(II) (20 mmol), potassium carbonate (405 mmol) and water (80 mL). The mixture was stirred at 80° C. for 1 hour under nitrogen. Upon completion, the reaction mixture was filtered and the filtrate was concentrated under reduced pressure. The crude mixture was partitioned between ethyl acetate and water and the aqueous phase was extracted with ethyl acetate. The combined organic layers were dried over sodium sulfate, filtered and concentrated under reduced pressure to give a residue. The residue was purified by prep-HPLC (SiO₂, petroleum ether:ethyl acetate 10:1) to yield the title compound. LCMS: 387.2.

Step 2: 3-(6-fluoropyridin-3-yl)piperidine-2,6-dione. Solution 1 consisted of 2,6-bis(benzyloxy)-6′-fluoro-3,3′-bipyridine (26 mmol) in tetrahydrofuran (150 mL) and 1,4-dioxane (150 mL). The volume of the flow reactor 1 (¼″ stainless steel tube, 5 mL) was filled with 5% palladium(II) hydroxide on alumina. The residence time of flow reactor 1 was 15 minutes and the bath temperature was 50° C. The flow rate of Pump 1 was adjusted to 0.3 mL/minute. The flow rate of H₂ was adjusted to 30 mL/minute at 1 MPa. Solution 1 ran in flow reactor 1 under 1 MPa and collected with a bottle. Upon completion, the reaction mixture was concentrated and triturated with methyl tert-butyl ether and used in the next step. LCMS: 209.1.

Step 3: tert-butyl 4-(2-(1-(5-(2,6-dioxopiperidin-3-yl)pyridin-2-yl)piperidin-4-yl)ethyl)piperidine-1-carboxylate. To a solution of tert-butyl 4-(2-(piperidin-4-yl)ethyl)piperidine-1-carboxylate hydrochloride (0.36 mmol) and 3-(6-fluoropyridin-3-yl)piperidine-2,6-dione (0.36 mmol) in N-methyl-2-pyrrolidone (1.0 mL) in a vial was added N,N-diisopropylethylamine (0.25 mL). The vial was sealed, and the reaction mixture was stirred at 120° C. overnight. The reaction mixture was then diluted with ethyl acetate (10 mL) and water (10 mL). The aqueous layer was extracted with ethyl acetate (3×10 mL). The organic layers were combined, washed with brine (20 mL), dried over anhydrous sodium sulfate, filtered, and concentrated under reduced pressure. The residue was purified by flash column chromatography on silica gel eluting with ethyl acetate/dichloromethane to afford the title compound. LCMS: 485.4.

Step 4: 3-(6-(4-(2-(piperidin-4-yl)ethyl)piperidin-1-yl)pyridin-3-yl)piperidine-2,6-dione (INT 27-1). Trifluoroacetic acid (0.12 mL) was added to a stirred solution of tert-butyl 4-(2-(1-(5-(2,6-dioxopiperidin-3-yl)pyridin-2-yl)piperidin-4-yl)ethyl)piperidine-1-carboxylate (0.16 mmol) in dichloromethane (3 mL) at room temperature. After stirring at room temperature for 1 hour, the reaction mixture was concentrated under reduced pressure to yield the title compound which was used without further purification. LCMS: 385.3.

### Intermediate 28-1.

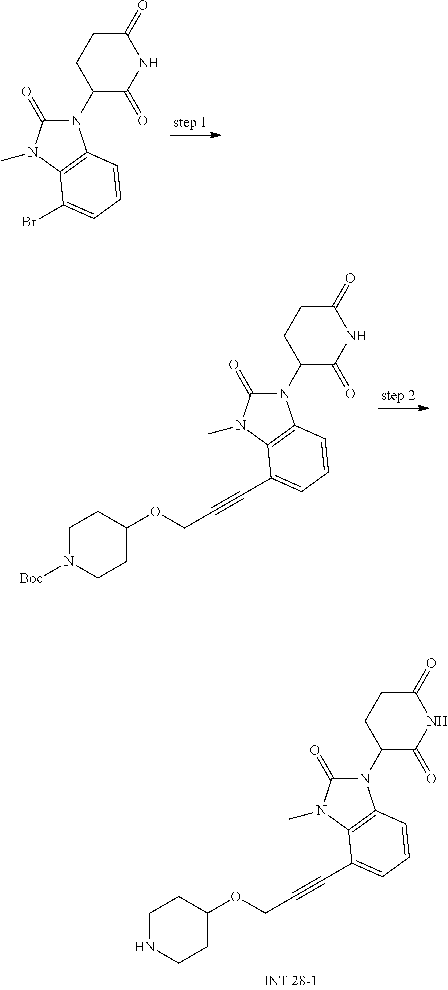 [SMILES: Cn1c(=O)n(C2CCC(=O)NC2=O)c2cccc(Br)c21 ; Cn1c(=O)n(C2CCC(=O)NC2=O)c2cccc(C#CCOC3CCN(C(=O)OC(C)(C)C)CC3)c21 ; Cn1c(=O)n(C2CCC(=O)NC2=O)c2cccc(C#CCOC3CCNCC3)c21]

Step 1: tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate. To a mixture of 3-(4-bromo-3-methyl-2-oxo-2,3-dihydro-1H-benzo[d]imidazol-1-yl)piperidine-2,6-dione (3.0 mmol) and tert-butyl 4-prop-2-ynoxypiperidine-1-carboxylate (4.4 mmol) in N,N-dimethylformamide (6 mL) was added copper(I) iodide (0.6 mmol), triethylamine (6.0 mmol) and tetrakis(triphenylphosphine)palladium(0) (0.15 mmol). The mixture was stirred at 80° C. for 12 hours under nitrogen. The reaction mixture was then cooled to room temperature, filtered and concentrated under reduced pressure to give a residue. The residue was purified by prep-HPLC (35% to 65% acetonitrile/water with 0.04% hydrochloric acid), and fractions containing the desired product were pooled and lyophilized to yield the title compound. LCMS: 441.2.

Step 2: 3-(3-methyl-2-oxo-4-(3-(piperidin-4-yloxy)prop-1-yn-1-yl)-2,3-dihydro-1H-benzo[d]imidazol-1-yl)piperidine-2,6-dione (INT 28-1). To the mixture of tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate (0.6 mmol) in dichloromethane (3 mL) was added trifluoroacetic (1 mL) and the mixture was stirred for 30 minutes at room temperature. Upon completion, the reaction mixture was concentrated and purified by prep-HPLC (5% to 45% acetonitrile/water with 0.1% trifluoroacetic acid), and fractions containing the desired product were pooled and lyophilized to yield the title compound. LCMS: 397.1.

### Intermediates 29-1-29-2.

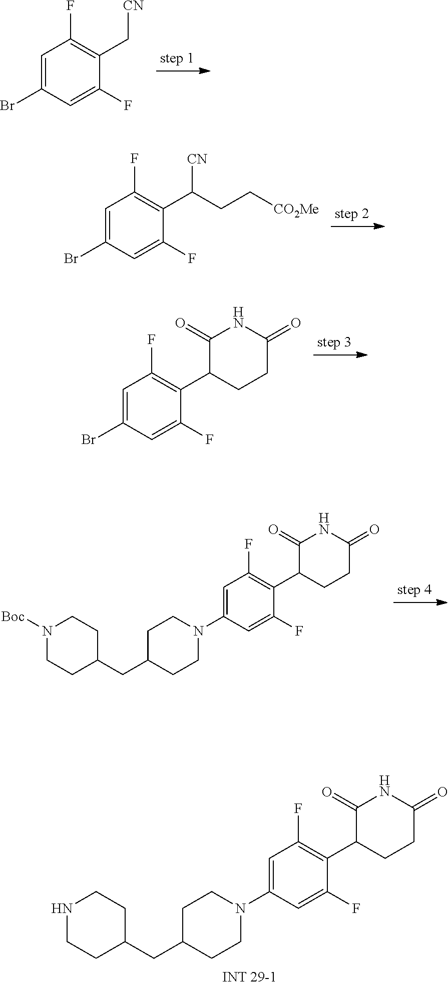 [SMILES: N#CCc1c(F)cc(Br)cc1F ; COC(=O)CCC(C#N)c1c(F)cc(Br)cc1F ; O=C1CCC(c2c(F)cc(Br)cc2F)C(=O)N1 ; CC(C)(C)OC(=O)N1CCC(CC2CCN(c3cc(F)c(C4CCC(=O)NC4=O)c(F)c3)CC2)CC1 ; O=C1CCC(c2c(F)cc(N3CCC(CC4CCNCC4)CC3)cc2F)C(=O)N1]

Step 1: methyl 4-(4-bromo-2,6-difluorophenyl)-4-cyanobutanoate. To a mixture of 2-(4-bromo-2,6-difluoro-phenyl)acetonitrile (21.5 mmol) in tetrahydrofuran (50 mL) was added lithium diisopropylamide (26 mmol) at −60° C. dropwise, then the mixture was stirred at room temperature for 30 minutes under nitrogen. Methyl 3-bromopropanoate (26 mmol) was added at −60° C. and the resulting mixture was stirred at room temperature for 12 hours under nitrogen. Upon completion, the reaction mixture was quenched by adding saturated aqueous ammonium chloride solution (30 mL) at 0° C. and extracted with ethyl acetate. The combined organic layers were dried over sodium sulfate, filtered and concentrated under reduced pressure to yield the title compound. LCMS: 318.0.

Step 2: 3-(4-bromo-2,6-difluorophenyl)piperidine-2,6-dione. To a mixture of methyl 4-(4-bromo-2,6-difluorophenyl)-4-cyanobutanoate (18.2 mmol) in acetic acid (20 mL) was added sulfuric acid (2 mL), then the mixture was stirred at 90° C. for 2 hours. The reaction mixture was then cooled to room temperature and slowly poured into ice water and filtered. The filter cake was lyophilized to yield the title compound. LCMS: 304.0.

Step 3: tert-butyl 4-((1-(4-(2,6-dioxopiperidin-3-yl)-3,5-difluorophenyl)piperidin-4-yl)methyl)piperidine-1-carboxylate. A mixture of 3-(4-bromo-2,6-difluorophenyl)piperidine-2,6-dione (3.3 mmol), tert-butyl 4-(piperidin-4-ylmethyl)piperidine-1-carboxylate (6.6 mmol), 2 dichloro[1,3-bis(2,6-di-3-pentylphenyl)imidazol-2-ylidene](3-chloropyridyl)palladium(II) (0.3 mmol), and cesium carbonate (6.6 mmol) in 1,4-dioxane (15 mL) was degassed with argon for 1 minute. The reaction mixture was stirred at 100° C. for 2 hours under an argon atmosphere. The reaction mixture was filtered through Celite and the filtrate was concentrated under reduced pressure. The residue was purified by prep-HPLC (50% to 90% acetonitrile/water with 0.04% hydrochloric acid), and fractions containing the desired product were pooled and lyophilized to yield the title compound. LCMS: 406.2.

Step 4: 3-(2,6-difluoro-4-(4-(piperidin-4-ylmethyl)piperidin-1-yl)phenyl)piperidine-2,6-dione (INT 29-1). To a mixture of tert-butyl 4-((1-(4-(2,6-dioxopiperidin-3-yl)-3,5-difluorophenyl)piperidin-4-yl)methyl)piperidine-1-carboxylate (1.6 mmol) in ethyl acetate (3 mL) was added hydrochloric acid in dioxane (1 mL, 4 M). The mixture was stirred at room temperature for 1 hour. The reaction mixture was concentrated under reduced pressure to give a residue, which was purified by triturating with ethyl acetate to afford the title compound. LCMS: 406.2.

The following intermediates were made in a similar fashion to INT 29-1 and are shown below in Table 2P. To prepare the below intermediates, different reagents/starting materials were used than previously described in the steps toward INT 29-1 and are noted in the last column of Table 2P—“Changes to Procedure: Different Reagents/Starting Materials”. A person of ordinary skill in the art will readily recognize which reagents/starting materials used for the synthesis of INT 29-1 were replaced with the different reagents/starting materials noted below.

TABLE 2P
Intermediates

| | | | Changes to Procedure: Different |
|---|---|---|---|
| Intermediate | Structure | LCMS | Reagents/Starting Materials |
| INT 29-2 | 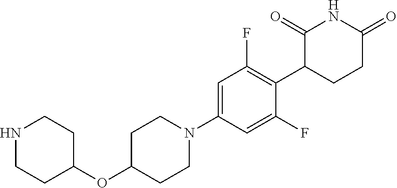 [SMILES: O=C1CCC(c2c(F)cc(N3CCC(OC4CCNCC4)CC3)cc2F)C(=O)N1] | 408.2 | tert-butyl 4-(piperidin-4- yloxy)piperidine-1-carboxylate in step 3 |

### Intermediate 30-1.

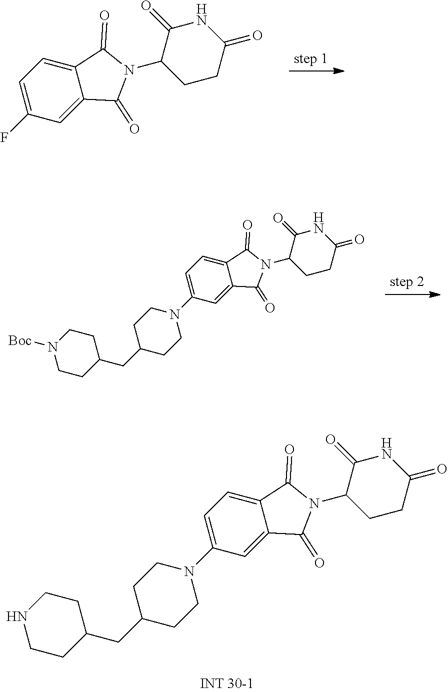 [SMILES: O=C1CCC(N2C(=O)c3ccc(F)cc3C2=O)C(=O)N1 ; CC(C)(C)OC(=O)N1CCC(CC2CCN(c3ccc4c(c3)C(=O)N(C3CCC(=O)NC3=O)C4=O)CC2)CC1 ; O=C1CCC(N2C(=O)c3ccc(N4CCC(CC5CCNCC5)CC4)cc3C2=O)C(=O)N1]

Step 1: tert-butyl 4-((1-(2-(2,6-dioxopiperidin-3-yl)-1,3-dioxoisoindolin-5-yl)piperidin-4-yl)methyl)piperidine-1-carboxylate. To a mixture of 2-(2,6-dioxopiperidin-3-yl)-5-fluoroisoindoline-1,3-dione (0.9 mmol) and tert-butyl 4-(4-piperidylmethyl)piperidine-1-carboxylate (0.9 mmol) in NMP (3 mL) was added DIPEA (0.47 mL). The vial was sealed, and the mixture was stirred at 120° C. for 16 h. The reaction mixture was diluted with EtOAc (20 mL) and water (20 mL) and transferred to a separatory funnel. The aqueous layer was extracted with EtOAc (3×20 mL). The combined organic layers were washed with brine, dried over Na₂SO₄, filtered, and concentrated under reduced pressure. The crude residue was purified by flash column chromatography eluting with EtOAc/hexane to yield the title compound. LCMS: 439.3

Step 2: 2-(2,6-dioxopiperidin-3-yl)-5-(4-(piperidin-4-ylmethyl)piperidin-1-yl)isoindoline-1,3-dione (INT 30-1) To tert-butyl 4-((1-(2-(2,6-dioxopiperidin-3-yl)-1,3-dioxoisoindolin-5-yl)piperidin-4-yl)methyl)piperidine-1-carboxylate (0.9 mmol) in dichloromethane (3 mL) was added trifluoroacetic acid (0.25 mL) dropwise. The reaction mixture was stirred at room temperature for 2 h, then diluted with MeCN and concentrated to yield the title compound which was used without further purification. LCMS: 439.3.

### Intermediates 31-1-31-5.

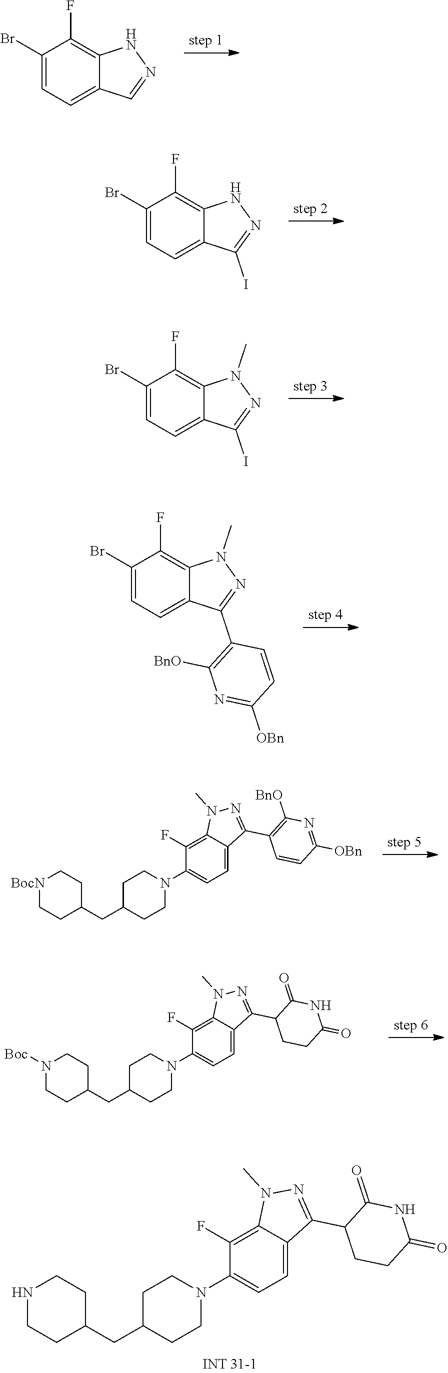

Step 1: 6-bromo-7-fluoro-3-iodo-1H-indazole. A mixture of 6-bromo-7-fluoro-1H-indazole (20.9 mmol), DMF (50 mL), iodine (23.0 mmol), and potassium hydroxide (83.7 mmol) was stirred at room temperature overnight. The mixture was diluted with water and adjusted to pH 7 with concentrated HCl. The aqueous layer was extracted with EtOAc twice, and the organic layer was washed with brine, filtered, and concentrated under reduced pressure. The residue was purified by purified by silica gel column chromatography eluting with EtOAc/petroleum ether to yield the title compound. LCMS: 339.0

Step 2: 6-bromo-7-fluoro-3-iodo-1-methyl-1H-indazole. K₂CO₃ (59 mmol) was added to a mixture of 6-bromo-7-fluoro-3-iodo-1H-indazole (19.5 mmol) and MeI (39.3 mmol) in DMF (75 mL), and the mixture was stirred at 40° C. for 3.5 hours under N₂ atmosphere. The reaction mixture was quenched by addition of saturated aqueous NH₄Cl at 0° C., and then diluted with water. The aqueous layer was extracted with EtOAc, and the combined organic layers were washed with water (20 mL×3), dried over Na₂SO₄, filtered, and concentrated under reduced pressure to give a residue. The residue was purified by column chromatography eluting with EtOAc/petroleum ether to yield the title compound.

Step 3: 3-(2,6-bis(benzyloxy)pyridin-3-yl)-6-bromo-7-fluoro-1-methyl-1H-indazole. To a solution of 6-bromo-7-fluoro-3-iodo-1-methyl-1H-indazole (5.6 mmol) in 10 mL 10:1 DMF/H2O was added 2,6-bis(benzyloxy)-3-(4,4,5,5-tetramethyl-1,3,2-dioxaborolan-2-yl)pyridine (6.7 mmol), Cs₂CO₃ (10.1 mmol) and Pd(dppf)Cl₂ (0.45 mmol). The mixture was degassed with N₂ three times and stirred at 95° C. for 16 h. The reaction mixture was diluted with water and extracted with EtOAc three times. The combined organic layers were washed with brine, dried over Na₂SO₄, concentrated under reduced pressure to yield the crude product, which was purified by column chromatography on silica gel (PE:EA=20:1) to yield the title compound. LCMS: 520.

Step 4: tert-butyl 4-((1-(3-(2,6-bis(benzyloxy)pyridin-3-yl)-7-fluoro-1-methyl-1H-indazol-6-yl)piperidin-4-yl)methyl)piperidine-1-carboxylate. A mixture of 6-bromo-3-(2,6-dibenzyloxy-3-pyridyl)-7-fluoro-1-methyl-indazole (0.48 mmol), tert-butyl 4-(4-piperidylmethyl)piperidine-1-carboxylate (0.58 mmol), and Cs₂CO₃ (1.45 mmol) in 1,4-dioxane (5 mL) was sparged with N₂ for 10 minutes. XPhos (0.024 mmol) and Pd₂(dba)₃ (0.015 mmol) were added, the vial was sealed and stirred at 100° C. for 16 h. The reaction mixture was cooled to room temperature, filtered through celite, and concentrated under reduced pressure. The crude residue was purified by silica gel chromatography eluting with EtOAc/hexane to yield the title compound. LCMS: 720.4

Step 5: tert-butyl 4-[[1-[3-(2,6-dioxo-3-piperidyl)-7-fluoro-1-methyl-indazol-6-yl]-4-piperidyl]methyl]piperidine-1-carboxylate. To tert-butyl 4-((1-(3-(2,6-bis(benzyloxy)pyridin-3-yl)-7-fluoro-1-methyl-1H-indazol-6-yl)piperidin-4-yl)methyl)piperidine-1-carboxylate (0.14 mmol) in EtOH (2 mL) was added palladium hydroxide on carbon, powder, unreduced, 20% Pd, moisture ca 60% (20 mg), and the reaction apparatus was evacuated and backfilled with H₂ three times. The reaction mixture was stirred under a H₂ balloon at room temperature for 16 h. The mixture was then filtered through celite. The filtrate was concentrated to yield the title compound which was used without further purification. LCMS: 542.4

Step 6: 3-[7-fluoro-1-methyl-6-[4-(4-piperidylmethyl)-1-piperidyl]indazol-3-yl]piperidine-2,6-dione (INT 31-1). To tert-butyl 4-[[1-[3-(2,6-dioxo-3-piperidyl)-7-fluoro-1-methyl-indazol-6-yl]-4-piperidyl]methyl]piperidine-1-carboxylate (0.12 mmol) in DCM (2 mL) was added trifluoroacetic acid (0.2 mL) dropwise at room temperature. The reaction mixture was stirred at room temperature for 1 h, then diluted with MeCN and concentrated under reduced pressure to yield the title compound which was used without further purification. LCMS: 442.3

The following intermediates were made in a similar fashion to TNT 31-1 and are shown below in Table 2Q. To prepare the below intermediates, different reagents/starting materials were used than previously described in the steps toward TNT 31-1 and are noted in the last column of Table 2Q—“Changes to Procedure: Different Reagents/Starting Materials”. A person of ordinary skill in the art will readily recognize which reagents/starting materials used for the synthesis of TNT 31-1 were replaced with the different reagents/starting materials noted below.

TABLE 2Q
Intermediates

| | | | Changes to Procedure: Different |
|---|---|---|---|
| Intermediate | Structure | LCMS | Reagents/Starting Materials |
| INT 31-2 | 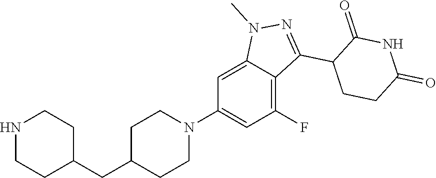 [SMILES: Cn1nc(C2CCC(=O)NC2=O)c2c(F)cc(N3CCC(CC4CCNCC4)CC3)cc21] | 442.3 | 6-bromo-4-fluoro-1H-indazole in step 1 |
| INT 31-3 | 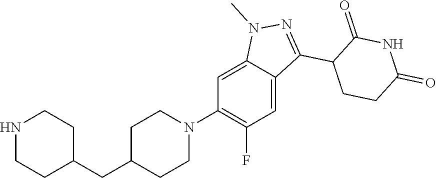 [SMILES: Cn1nc(C2CCC(=O)NC2=O)c2cc(F)c(N3CCC(CC4CCNCC4)CC3)cc21] | 442.3 | 6-bromo-5-fluoro-3-iodo-1- methyl-1H-indazole in step 3 |
| INT 31-4 | 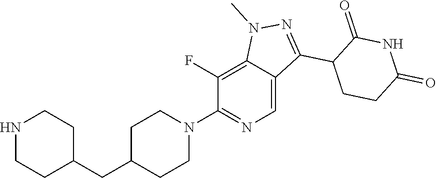 [SMILES: Cn1nc(C2CCC(=O)NC2=O)c2cnc(N3CCC(CC4CCNCC4)CC3)c(F)c21] | 443.1 | 6-chloro-7-fluoro-1H-pyrazolo[4,3- c]pyridine in step 1 |
| INT 31-5 | 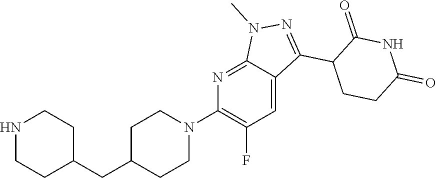 [SMILES: Cn1nc(C2CCC(=O)NC2=O)c2cc(F)c(N3CCC(CC4CCNCC4)CC3)nc21] | 443.1 | 6-chloro-5-fluoro-3-iodo-1- methyl-1H- pyrazolo[3,4-b]pyridine in step 1 |

### Intermediate 32-1.

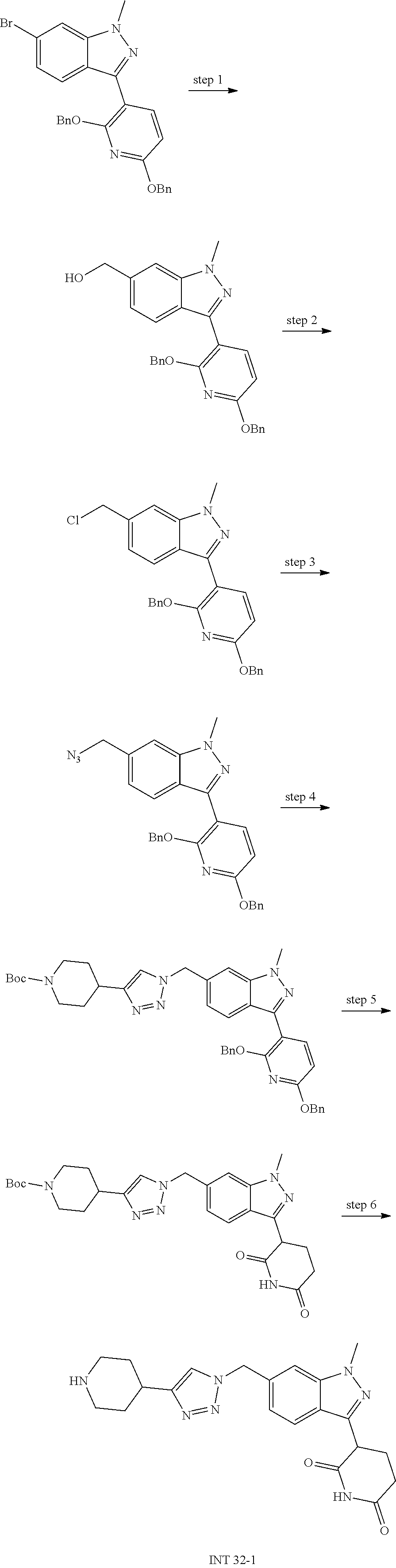

Step 1: (3-(2,6-bis(benzyloxy)pyridin-3-yl)-1-methyl-1H-indazol-6-yl)methanol. A mixture of 3-(2,6-bis(benzyloxy)pyridin-3-yl)-6-bromo-1-methyl-1H-indazole (13.0 mmol) and (tributylstannyl)methanol (19.5 mmol) in 1,4-dioxane (60.0 mL) was sparged with N₂ for 5 min. XPhos Pd G2 (1.4 mmol) was added, then the reaction mixture was stirred at 80° C. for 16 h. Upon completion, the reaction, the reaction mixture was concentrated under reduced pressure. The crude residue was purified by silica gel chromatography eluting with 40-50% EtOAc/petroleum ether to yield the title compound. LCMS: 452.4

Step 2: 3-(2,6-bis(benzyloxy)pyridin-3-yl)-6-(chloromethyl)-1-methyl-1H-indazole. A stirred solution of (3-(2,6-bis(benzyloxy)pyridin-3-yl)-1-methyl-1H-indazol-6-yl)methanol (12.2 mmol) in DCM (50 mL) under N₂ was cooled to 0° C. N,N-diisopropylethylamine (5.3 mL, 36.5 mmol) & methanesulfonyl chloride (1.9 mL, 24.4 mmol) were added to the reaction mixture at 0° C. The reaction mixture was stirred at room temperature for 6 h. Upon completion, the reaction mixture was quenched with water and diluted with DCM. The aqueous layer was extracted with DCM, then the combined organic layer was dried over anhydrous sodium sulfate, filtered, and concentrated under reduced pressure to yield the title compound which was used without further purification. LCMS: 470.22

Step 3: 6-(azidomethyl)-3-(2,6-bis(benzyloxy)pyridin-3-yl)-1-methyl-1H-indazole. To a stirred solution of 3-(2,6-bis(benzyloxy)pyridin-3-yl)-6-(chloromethyl)-1-methyl-1H-indazole (4.3 mmol) in DMF (20 mL) was added sodium azide (8.5 mmol). The reaction mixture was stirred at room temperature for 6 h. Upon completion, the reaction mixture was quenched with water and diluted with DCM. The aqueous layer was extracted with DCM, then the combined organic layer was dried over anhydrous sodium sulfate, filtered, and concentrated under reduced pressure to yield the title compound which was used without further purification. LCMS: 477.2

Step 4: tert-butyl 4-(1-((3-(2,6-bis(benzyloxy)pyridin-3-yl)-1-methyl-1H-indazol-6-yl)methyl)-1H-1,2,3-triazol-4-yl)piperidine-1-carboxylate. To a stirred solution of 6-(azidomethyl)-3-(2,6-bis(benzyloxy)pyridin-3-yl)-1-methyl-1H-indazole (3.4 mmol) & tert-butyl 4-ethynylpiperidine-1-carboxylate (6.7 mmol) in tert-butyl alcohol (10 mL) & water (10 mL). L-ascorbic acid sodium salt (1.9 mmol) & copper(II) sulfate pentahydrate (3.4 mmol) were added at rt. The reaction mixture was then stirred at 70° C. for 6 h. Upon completion, the reaction mixture was quenched with water and diluted with EtOAc. The aqueous layer was extracted with EtOAc, then the combined organic layer was dried over anhydrous sodium sulfate, filtered, and concentrated under reduced pressure. The crude residue was purified by silica gel chromatography eluting with 60-100% EtOAc/petroleum ether to yield the title compound. LCMS: 577.0

Step 5: tert-butyl 4-(1-((3-(2,6-dioxopiperidin-3-yl)-1-methyl-1H-indazol-6-yl)methyl)-1H-1,2,3-triazol-4-yl)piperidine-1-carboxylate. To a stirred solution of tert-butyl 4-(1-((3-(2,6-bis(benzyloxy)pyridin-3-yl)-1-methyl-1H-indazol-6-yl)methyl)-1H-1,2,3-triazol-4-yl)piperidine-1-carboxylate (1.9 mmol) in THE (20 mL) was added palladium hydroxide on carbon, powder, unreduced, 20% Pd, moisture ca 60% (1.0 g). The reaction apparatus was evacuated and backfilled with H₂ three times. The reaction mixture was stirred under an atmosphere of H₂ (15 psi) at room temperature for 6 h. The mixture was then filtered through celite. The filtrate was concentrated under reduced pressure. The crude residue was purified by silica gel chromatography eluting with 1-8% MeOH/DCM to yield the title compound. LCMS: 508.3

Step 6: 3-(1-methyl-6-((4-(piperidin-4-yl)-1H-1,2,3-triazol-1-yl)methyl)-1H-indazol-3-yl)piperidine-2,6-dione (INT 32-1). To a stirred solution of tert-butyl 4-(1-((3-(2,6-bis(benzyloxy)pyridin-3-yl)-1-methyl-1H-indazol-6-yl)methyl)-1H-1,2,3-triazol-4-yl)piperidine-1-carboxylate (1.4 mmol) in DCM (10 mL) was added trifluoroacetic acid (1.12 mL, 13.8 mmol) dropwise at room temperature. The reaction mixture was stirred at room temperature for 3 h. Upon completion the reaction mixture was concentrated under reduced pressure. The crude residue was washed with diethyl ether. The resulting solids were dissolved in THF, DCM, ACN (1:1:1) and then neutralized using Amberlyst A21 resin. The mixture was then filtered and rinsed with mixture solvents THF, DCM, ACN (1:1:1). The filtrate was concentrated under reduced pressure to yield the title compound. LCMS: 408.3.

### Intermediates 33-1-33-2.

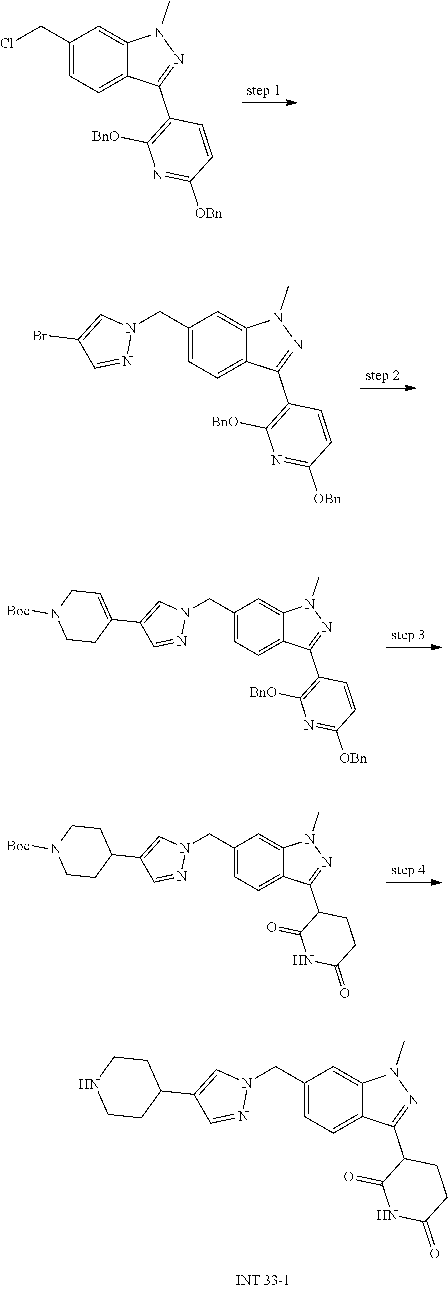 [SMILES: Cn1nc(-c2ccc(OCc3ccccc3)nc2OCc2ccccc2)c2ccc(CCl)cc21 ; Cn1nc(-c2ccc(OCc3ccccc3)nc2OCc2ccccc2)c2ccc(Cn3cc(Br)cn3)cc21 ; Cn1nc(-c2ccc(OCc3ccccc3)nc2OCc2ccccc2)c2ccc(Cn3cc(C4=CCN(C(=O)OC(C)(C)C)CC4)cn3)cc21 ; Cn1nc(C2CCC(=O)NC2=O)c2ccc(Cn3cc(C4CCN(C(=O)OC(C)(C)C)CC4)cn3)cc21 ; Cn1nc(C2CCC(=O)NC2=O)c2ccc(Cn3cc(C4CCNCC4)cn3)cc21]

Step 1: 3-(2,6-bis(benzyloxy)pyridin-3-yl)-6-((4-bromo-1H-pyrazol-1-yl)methyl)-1-methyl-1H-indazole. To a stirred solution of 3-(2,6-bis(benzyloxy)pyridin-3-yl)-6-(chloromethyl)-1-methyl-1H-indazole (3.8 mmol) and 4-bromo-1H-pyrazole (3.8 mmol) in DMF (20 mL) under N₂ was added sodium hydride (11.3 mmol) at room temperature. The reaction mixture was then stirred at 80° C. for 6 h. Upon completion, the reaction mixture was quenched with water and diluted with ethyl acetate. The aqueous layer was extracted with EtOAc, then the combined organic layer was dried over anhydrous sodium sulfate, filtered, and concentrated under reduced pressure. The crude residue was purified by silica gel chromatography by eluting with 40-50% EtOAc/petroleum ether to yield the title compound. LCMS: 580.4.

Step 2: tert-butyl 4-(1-((3-(2,6-bis(benzyloxy)pyridin-3-yl)-1-methyl-1H-indazol-6-yl)methyl)-1H-pyrazol-4-yl)-3,6-dihydropyridine-1(2H)-carboxylate. A mixture of 3-(2,6-bis(benzyloxy)pyridin-3-yl)-6-((4-bromo-1H-pyrazol-1-yl)methyl)-1-methyl-1H-indazole (3.1 mmol) and tert-butyl 4-(4,4,5,5-tetramethyl-1,3,2-dioxaborolan-2-yl)-3,6-dihydropyridine-1(2H)-carboxylate (6.2 mmol) in 1,4-dioxane (20 mL) & water (4 mL) was sparged with nitrogen for 5 min. Cs₂CO₃ (9.3 mmol) was added under N₂, followed by XPhos Pd G3 (0.3 mmol). The reaction mixture was sparged with nitrogen for an additional 2 min, then the reaction mixture was stirred at 100° C. for 16 h. Upon completion, the reaction mixture was quenched with water and diluted with ethyl acetate. The aqueous layer was extracted with EtOAc, then the combined organic layer was dried over anhydrous sodium sulfate, filtered, and concentrated under reduced pressure. The crude residue was purified by silica gel chromatography by eluting with 40-50% EtOAc/petroleum ether to yield the title compound. LCMS: 683.3.

Step 3: tert-butyl 4-(1-((3-(2,6-dioxopiperidin-3-yl)-1-methyl-1H-indazol-6-yl)methyl)-1H-pyrazol-4-yl)piperidine-1-carboxylate. To a stirred solution of tert-butyl 4-(1-((3-(2,6-bis(benzyloxy)pyridin-3-yl)-1-methyl-1H-indazol-6-yl)methyl)-1H-pyrazol-4-yl)-3,6-dihydropyridine-1(2H)-carboxylate (1.9 mmol) in THE (10 mL) was added palladium hydroxide on carbon, powder, unreduced, 20% Pd, moisture ca 60% (1.3 g). The reaction mixture was stirred under an atmosphere of H₂ at room temperature for 6 h. Upon completion, the reaction apparatus was evacuated and backfilled with N₂ three times, and the mixture was filtered over celite. The celite pad was rinsed with EtOAc, and the filtrate was concentrated under reduced pressure to yield the title compound which was used without further purification.

Step 4: 3-(1-methyl-6-((4-(piperidin-4-yl)-1H-pyrazol-1-yl)methyl)-1H-indazol-3-yl)piperidine-2,6-dione (INT 33-1). To a stirred solution of tert-butyl 4-(1-((3-(2,6-bis(benzyloxy)pyridin-3-yl)-1-methyl-1H-indazol-6-yl)methyl)-1H-1,2,3-triazol-4-yl)piperidine-1-carboxylate (1.2 mmol) in DCM (10 mL) was added trifluoroacetic acid (0.9 mL, 11.7 mmol) at 0° C. The reaction mixture was then stirred at room temperature for 3 h. Upon completion, the reaction mixture was diluted with EtOAc and concentrated under reduced pressure. The crude residue was washed with diethyl ether and dried under reduced pressure. The residue was then dissolved in 20% IPA/DCM then Amberlyst A21 resin was added and the reaction mixture was stirred for 1 h. The mixture was filtered with 20% IPA/DCM. The filtrate was concentrated under reduced pressure to yield the title compound. LCMS: 407.4.

The following intermediates were made in a similar fashion to INT 33-1 and are shown below in Table 2R. To prepare the below intermediates, different reagents/starting materials were used than previously described in the steps toward INT 33-1 and are noted in the last column of Table 2R—“Changes to Procedure: Different Reagents/Starting Materials”. A person of ordinary skill in the art will readily recognize which reagents/starting materials used for the synthesis of INT 33-1 were replaced with the different reagents/starting materials noted below.

TABLE 2R
Intermediates

| | | | Changes to Procedure: Different |
|---|---|---|---|
| Intermediate | Structure | LCMS | Reagents/Starting Materials |
| INT 33-2 | 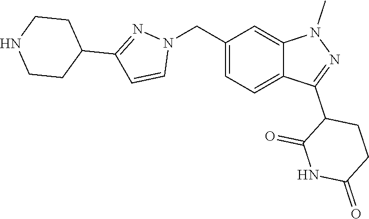 [SMILES: Cn1nc(C2CCC(=O)NC2=O)c2ccc(Cn3ccc(C4CCNCC4)n3)cc21] | 407.3 | 3-bromo-1H-pyrazole in step 1 |

### Intermediates 34-1A-D-35-1A-D.

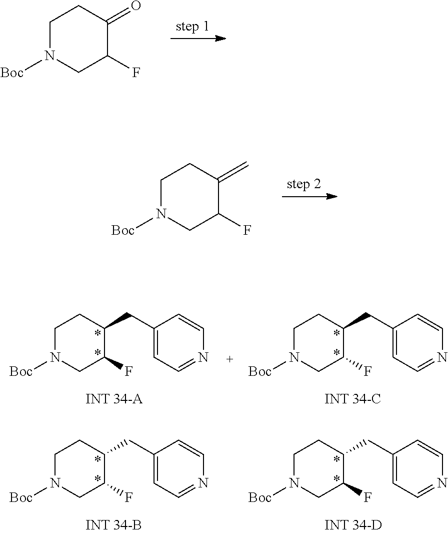 [SMILES: CC(C)(C)OC(=O)N1CCC(=O)C(F)C1 ; C=C1CCN(C(=O)OC(C)(C)C)CC1F ; CC(C)(C)OC(=O)N1CC[C@@H](Cc2ccncc2)[C@@H](F)C1 ; CC(C)(C)OC(=O)N1CC[C@@H](Cc2ccncc2)[C@H](F)C1 ; CC(C)(C)OC(=O)N1CC[C@H](Cc2ccncc2)[C@H](F)C1 ; CC(C)(C)OC(=O)N1CC[C@H](Cc2ccncc2)[C@@H](F)C1]

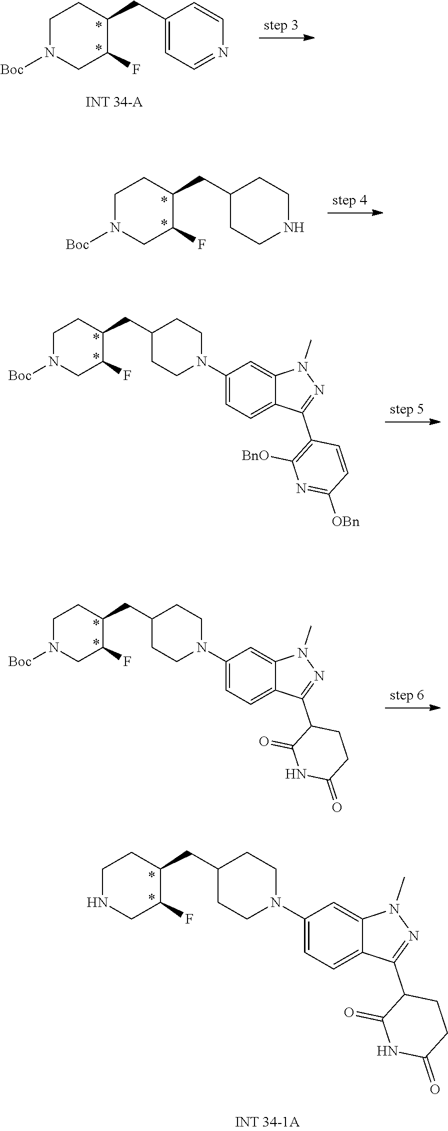 [SMILES: CC(C)(C)OC(=O)N1CC[C@@H](Cc2ccncc2)[C@@H](F)C1 ; CC(C)(C)OC(=O)N1CC[C@@H](CC2CCNCC2)[C@@H](F)C1 ; Cn1nc(-c2ccc(OCc3ccccc3)nc2OCc2ccccc2)c2ccc(N3CCC(C[C@@H]4CCN(C(=O)OC(C)(C)C)C[C@@H]4F)CC3)cc21 ; Cn1nc(C2CCC(=O)NC2=O)c2ccc(N3CCC(C[C@@H]4CCN(C(=O)OC(C)(C)C)C[C@@H]4F)CC3)cc21 ; Cn1nc(C2CCC(=O)NC2=O)c2ccc(N3CCC(C[C@@H]4CCNC[C@@H]4F)CC3)cc21]

Step 1: tert-butyl 3-fluoro-4-methylenepiperidine-1-carboxylate. To a stirred solution of methyltriphenylphosphonium bromide (460 mmol) in tetrahydrofuran (300 mL) at room temperature under nitrogen atmosphere was added potassium tert-butoxide (460 mmol), and the reaction mixture was stirred for 30 min. Then, a solution of tert-butyl 3-fluoro-4-oxopiperidine-1-carboxylate (230 mmol) in tetrahydrofuran (200 mL) was added dropwise, and the reaction mixture was stirred at room temperature for 1 h. Upon completion, the reaction mixture was diluted with water (300 mL) and extracted with ethyl acetate (3×500 mL). The organic fractions were separated and dried over anhydrous sodium sulfate, filtered, and concentrated. The residue was purified by silica gel column chromatography eluting with 10% EtOAc/hexanes yield the title compound.

Step 2: tert-butyl (3R*,4R*)-3-fluoro-4-(pyridin-4-ylmethyl)piperidine-1-carboxylate (INT 34-A). tert-butyl 3-fluoro-4-methylenepiperidine-1-carboxylate (27.9 mmol) was added to 9-BBN (0.5 M THE solution, 60 mL) at room temperature. The reaction mixture was stirred at 60° C. for 2 h under nitrogen atmosphere. Then the reaction mixture was cooled to room temperature. 4-bromopyridine (27.9 mmol) potassium carbonate (36.2 mmol) were added to the reaction mixture, followed by 1,1′-bis(diphenylphosphino)ferrocene-palladium(II)dichloride dichloromethane complex (0.27 mmol), N,N-dimethylformamide (48 mL) and water (12 mL). Then the reaction mixture was stirred at 60° C. under nitrogen atmosphere for 48 h. After completion of the reaction, the mixture was cooled to room temperature and poured into water (100 mL). The pH was adjusted to 11 with 10% aq. NaOH. The mixture was extracted with ethyl acetate (2×300 mL). The combined organic layers were dried over Na₂SO₄, filtered, and concentrated under reduced pressure. The residue was purified by silica gel column chromatography eluting with 70% EtOAc/petroleum ether yielding cis and trans isomer, which were subsequently further separated by chiral SFC (Chiralpak AD-H 150×25×5 μm, 80% CO₂/20% MeOH, 80 g/min) into four isomers, arbitrarily labeled as INT 34-A, INT 34-B, INT 34-C, INT 34-D. INT 34-A: tert-butyl (3R*,4R*)-3-fluoro-4-(pyridin-4-ylmethyl)piperidine-1-carboxylate LCMS: 295.0. INT 34-B: tert-butyl (3S*,4S*)-3-fluoro-4-(pyridin-4-ylmethyl)piperidine-1-carboxylate. INT 34-C: tert-butyl (3S*,4R*)-3-fluoro-4-(pyridin-4-ylmethyl)piperidine-1-carboxylate. INT 34-D: tert-butyl (3R*,4S*)-3-fluoro-4-(pyridin-4-ylmethyl)piperidine-1-carboxylate.

Step 3: tert-butyl (3R*,4R*)-3-fluoro-4-(piperidin-4-ylmethyl)piperidine-1-carboxylate. To a stirred solution of tert-butyl (3R*,4R*)-3-fluoro-4-(pyridin-4-ylmethyl) piperidine-1-carboxylate INT 34-A (0.6 g, 2.04 mmol, 1.0 eq.) in EtOH (10 mL) was added acetic acid (0.18 mL) dropwise followed by platinum (IV) oxide (0.61 mmol). The reaction mixture was then stirred at room temperature for 24 h under a hydrogen balloon. Upon completion, the reaction mixture was filtered over a celite pad, washed multiple times with ethanol and concentrated to yield the title compound which was used without further purification. LCMS: 300.0

Step 4: tert-butyl (3R*,4R*)-4-((1-(3-(2,6-bis(benzyloxy)pyridin-3-yl)-1-methyl-1H-indazol-6-yl)piperidin-4-yl)methyl)-3-fluoropiperidine-1-carboxylate. A mixture of tert-butyl (3R*,4R*)-3-fluoro-4-(piperidin-4-ylmethyl) piperidine-1-carboxylate (1.6 mmol) and 3-(2,6-bis(benzyloxy)pyridin-3-yl)-6-bromo-1-methyl-1H-indazole (1.6 mmol) in 1,4-dioxane (10 mL) was sparged with nitrogen for 5 mins. Cs2CO3 (4.9 mmol) was added under N2 atmosphere. X-Phos (0.16 mmol) followed by tris(dibenzylideneacetone) dipalladium(0) (0.08 mmol) were added under nitrogen atmosphere. Then the mixture was stirred at 90° C. for 16 h. Upon completion, the reaction mixture was filtered through celite and concentrated. The residue was purified by silica gel column chromatography eluting with 50% EtOAc/petroleum ether to yield the title compound. LCMS: 720.0.

Step 5: tert-butyl (3R*,4R*)-4-((1-(3-(2,6-dioxopiperidin-3-yl)-1-methyl-1H-indazol-6-yl)piperidin-4-yl)methyl)-3-fluoropiperidine-1-carboxylate. To a stirred solution of tert-butyl (3R*,4R*)-4-((1-(3-(2,6-bis(benzyloxy)pyridin-3-yl)-1-methyl-1H-indazol-6-yl) piperidin-4-yl) methyl)-3-fluoropiperidine-1-carboxylate (0.83 mmol) in tetrahydrofuran (10 mL) was added palladium hydroxide on carbon, powder, unreduced, 20% Pd, moisture ca 60% (0.6 g) and the reaction mixture was under a hydrogen balloon at room temperature for 16 h. Upon completion, the reaction mixture was filtered through celite, and the filtrate was concentrated to yield the title compound which was used without further purification. LCMS: 542.4.

Step 6: 3-(6-(4-(((3R*,4R*)-3-fluoropiperidin-4-yl)methyl)piperidin-1-yl)-1-methyl-1H-indazol-3-yl)piperidine-2,6-dione (INT 34-1A). tert-butyl (3R*,4R*)-4-((1-(3-(2,6-dioxopiperidin-3-yl)-1-methyl-1H-indazol-6-yl)piperidin-4-yl)methyl)-3-fluoropiperidine-1-carboxylate (0.13 mmol) was dissolved in DCM (3 mL), then trifluoroacetic acid (0.5 mL) was added dropwise at room temperature. The reaction mixture was stirred at room temperature for 20 min, then diluted with MeCN and concentrated under reduced pressure to yield the title compound which was used without further purification. LCMS: 458.2.

The following intermediates were made in a similar fashion to INT 34-1A and are shown below in Table 2S. To prepare the below intermediates, different reagents/starting materials were used than previously described in the steps toward INT 34-1A and are noted in the last column of Table 2S—“Changes to Procedure: Different Reagents/Starting Materials”. A person of ordinary skill in the art will readily recognize which reagents/starting materials used for the synthesis of INT 34-1A were replaced with the different reagents/starting materials noted below.

TABLE 2S
Intermediates

| | | | Changes to Procedure: Different |
|---|---|---|---|
| Intermediate | Structure | LCMS | Reagents/Starting Materials |
| INT 34-1B | 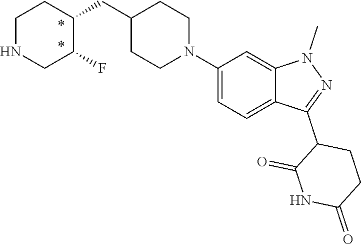 [SMILES: Cn1nc(C2CCC(=O)NC2=O)c2ccc(N3CCC(C[C@H]4CCNC[C@H]4F)CC3)cc21] | 458.2 | INT 34-B in step 3 |
| INT 34-1C | 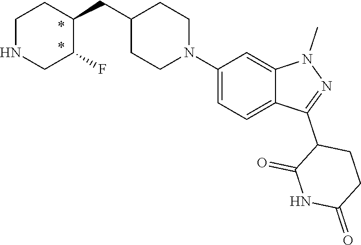 [SMILES: Cn1nc(C2CCC(=O)NC2=O)c2ccc(N3CCC(C[C@@H]4CCNC[C@H]4F)CC3)cc21] | 458.2 | INT 34-C in step 3 |
| INT 34-1D | 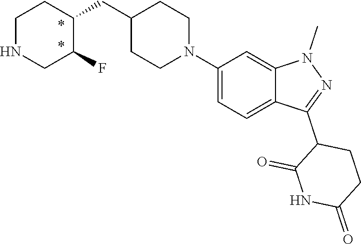 [SMILES: Cn1nc(C2CCC(=O)NC2=O)c2ccc(N3CCC(C[C@H]4CCNC[C@@H]4F)CC3)cc21] | 458.2 | INT 34-D in step 3 |
| INT 35-1A | 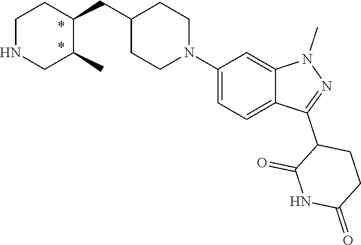 [SMILES: C[C@H]1CNCC[C@H]1CC1CCN(c2ccc3c(C4CCC(=O)NC4=O)nn(C)c3c2)CC1] | 438.3 | tert-butyl 3-methyl-4-oxopiperidine-1- carboxylate in step 1. The cis- and trans- isomers separated by silica gel column chromatography in step 2. RuPhos (5 mol %)/Pd(OAc)₂ (5 mol %)/NaOtBu (200 mol %) used in step 4. The diastereomers were spaerated via prep-SFC (Chiral PAK IK 250x30x5 μm, 65% CO₂/35% (0.5% MeONH3 IN ACN:IPA) (1:1) + 5% MeOH, 100 g/min) in step 4. |
| INT 35-1B | 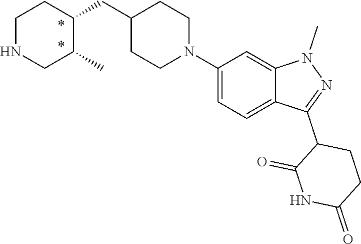 [SMILES: C[C@@H]1CNCC[C@@H]1CC1CCN(c2ccc3c(C4CCC(=O)NC4=O)nn(C)c3c2)CC1] | 438.3 | tert-butyl 3-methyl-4-oxopiperidine-1- carboxylate in step 1. The cis- and trans- isomers separated by silica gel column chromatography in step 2. RuPhos (5 mol %)/Pd(OAc)₂ (5 mol %)/NaOtBu (200 mol %) used in step 4. The diastereomers were spaerated via prep-SFC (Chiral PAK IK 250x30x5 μm, 65% CO₂/35% (0.5% MeONH3 IN ACN:IPA) (1:1) + 5% MeOH, 100 g/min) in step 4. |
| INT-35-1C | 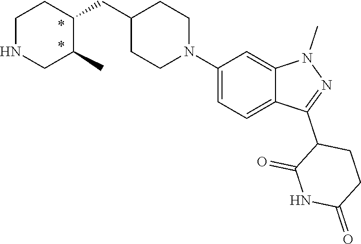 [SMILES: C[C@H]1CNCC[C@@H]1CC1CCN(c2ccc3c(C4CCC(=O)NC4=O)nn(C)c3c2)CC1] | 438.3 | tert-butyl 3-methyl-4-oxopiperidine-1- carboxylate in step 1. The cis- and trans- isomers separated by silica gel column chromatography in step 2. RuPhos (5 mol %)/Pd(OAc)₂ (5 mol %)/NaOtBu (200 mol %) used in step 4. The diastereomers were spaerated via prep-SFC (Chiral PAK IK 250x30x5 μm, 65% CO₂/35% (0.5% MeONH3 IN ACN:IPA) (1:1) + 5% MeOH, 100 g/min) in step 4. |
| INT-35-1D | 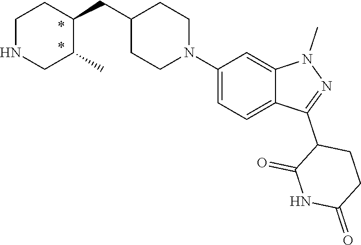 [SMILES: C[C@@H]1CNCC[C@H]1CC1CCN(c2ccc3c(C4CCC(=O)NC4=O)nn(C)c3c2)CC1] | 438.3 | tert-butyl 3-methyl-4-oxopiperidine-1- carboxylate in step 1. The cis- and trans- isomers separated by silica gel column chromatography in step 2. RuPhos (5 mol %)/Pd(OAc)₂ (5 mol %)/NaOtBu (200 mol %) used in step 4. The diastereomers were spaerated via prep-SFC (Chiral PAK IK 250x30x5 μm, 65% CO₂/35% (0.5% MeONH3 IN ACN:IPA) (1:1) + 5% MeOH, 100 g/min) in step 4. |

### Intermediates 36-1-36-6.

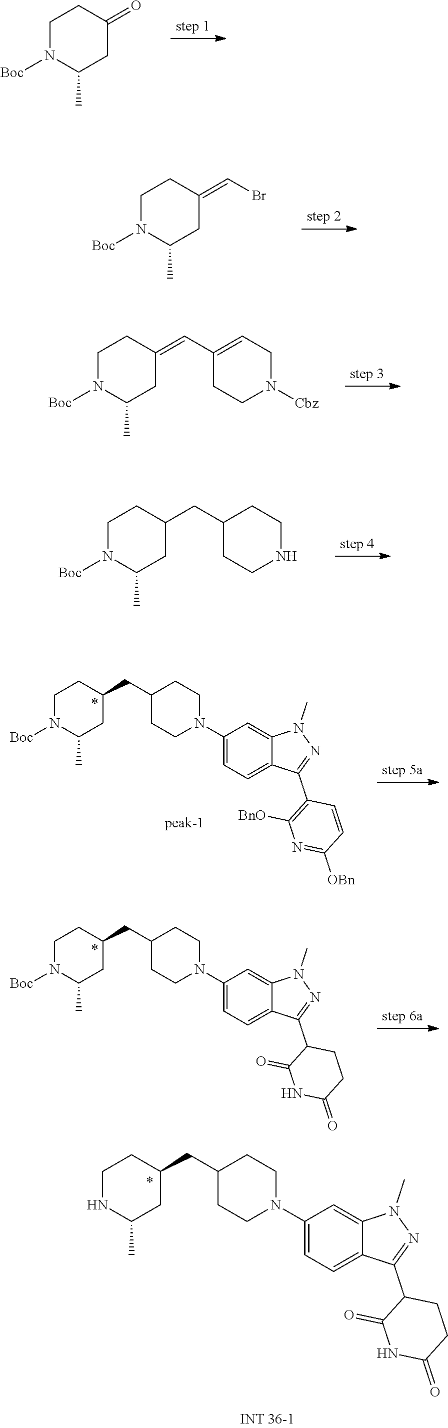

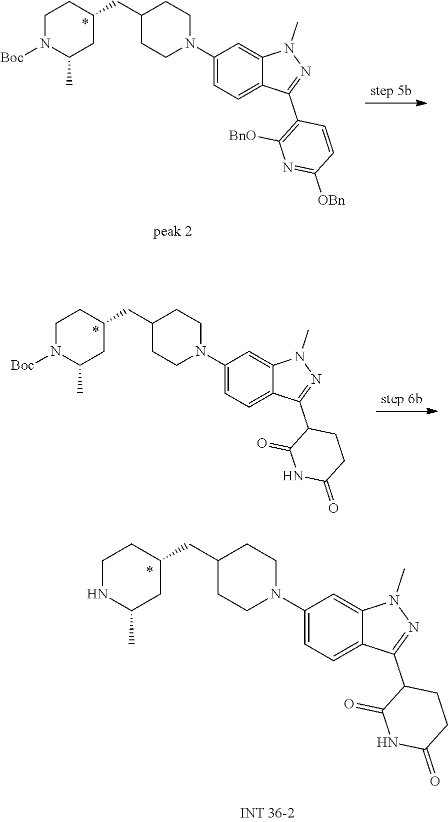 [SMILES: C[C@H]1C[C@@H](CC2CCN(c3ccc4c(-c5ccc(OCc6ccccc6)nc5OCc5ccccc5)nn(C)c4c3)CC2)CCN1C(=O)OC(C)(C)C ; C[C@H]1C[C@@H](CC2CCN(c3ccc4c(C5CCC(=O)NC5=O)nn(C)c4c3)CC2)CCN1C(=O)OC(C)(C)C ; C[C@H]1C[C@@H](CC2CCN(c3ccc4c(C5CCC(=O)NC5=O)nn(C)c4c3)CC2)CCN1]

Step 1: tert-butyl (S)-4-(bromomethylene)-2-methylpiperidine-1-carboxylate. A suspension of tert-butyl (2S)-2-methyl-4-oxo-piperidine-1-carboxylate (0.68 mmol) in THE (1.9 mL) was cooled in an ice bath with salt. A solution of lithium bis(trimethylsilyl)amide (1 M in THF, 0.87 mL) was added dropwise, then the reaction mixture was stirred at 0° C. for 30 minutes. tert-butyl (2S)-2-methyl-4-oxo-piperidine-1-carboxylate (0.47 mmol) was added in one portion, and then the reaction mixture was stirred at 0° C. for 1 h. Upon completion, the reaction mixture was quenched with saturated aqueous NH₄Cl, diluted with water/EtOAc, and transferred to a separatory funnel. The aqueous layer was extracted with EtOAc (3×10 mL). The combined organic layer was washed with brine, dried over anhydrous Na₂SO₄, filtered, and concentrated under reduced pressure. The crude residue was purified by column chromatography with silica gel, eluting with 0-15% EtOAc/hexane to yield the title compound.

Step 2: tert-butyl (2S)-4-[(1-benzyloxycarbonyl-3,6-dihydro-2H-pyridin-4-yl)methylene]-2-methyl-piperidine-1-carboxylate. A mixture of tert-butyl (2S)-4-(bromomethylene)-2-methyl-piperidine-1-carboxylate (0.4 mmol), benzyl 4-(4,4,5,5-tetramethyl-1,3,2-dioxaborolan-2-yl)-3,6-dihydro-2H-pyridine-1-carboxylate (1.0 mmol), and Cs₂CO₃ (1.2 mmol) in DMF (4 mL) was sparged with N₂ for 10 min. Pd(dppf)Cl₂ (0.08 mmol) was added, and the vial was sealed and heated to 80° C. for 16 h. Upon completion, the reaction mixture was cooled to room temperature and filtered through celite, rinsing with Et₂O. The filtrate was transferred to a separatory funnel and diluted with water (15 mL), and the aqueous layer was extracted with Et₂O (3×10 mL). The combined organic layer was washed with brine (2×10 mL), dried over Na₂SO₄, filtered, and concentrated under reduced pressure. The crude residue was purified by column chromatography with silica gel, eluting with 0-35% EtOAc/hexane to yield the title compound. LCMS: 327.3

Step 3: tert-butyl (2S)-2-methyl-4-(4-piperidylmethyl)piperidine-1-carboxylate. To tert-butyl (2S)-4-[(1-benzyloxycarbonyl-3,6-dihydro-2H-pyridin-4-yl)methylene]-2-methyl-piperidine-1-carboxylate (2.6 mmol) in EtOH (12 mL) was added palladium hydroxide on carbon, powder, unreduced, 20% Pd, moisture ca 60% (725 mg), and the reaction apparatus was evacuated and backfilled with H₂ three times. The reaction mixture was stirred under an atmosphere of H₂ (15 psi) at room temperature for 16 h. The mixture was then filtered through celite. The filtrate was concentrated under reduced pressure. The residue was purified by silica gel chromatography eluting with MeOH/DCM to yield the title compound. LCMS: 297.3

Step 4: tert-butyl (2S)-4-[[1-[3-(2,6-dibenzyloxy-3-pyridyl)-1-methyl-indazol-6-yl]-4-piperidyl]methyl]-2-methyl-piperidine-1-carboxylate. A mixture of 6-bromo-3-(2,6-dibenzyloxy-3-pyridyl)-1-methyl-indazole (0.65 mmol), tert-butyl (2S)-2-methyl-4-(4-piperidylmethyl)piperidine-1-carboxylate (0.78 mmol), and Cs₂CO₃ (1.95 mmol) in 1,4-dioxane (5.4 mL) was sparged with N₂ for 10 minutes. XPhos (0.033 mmol) and Pd₂(dba)₃ (0.02 mmol) were added, and the reaction mixture was then stirred at 100° C. for 16 h. The reaction mixture was cooled to room temperature, filtered over celite, and concentrated under reduced pressure. The residue was purified by column chromatography to yield the title compound as a mixture of diastereomers. This mixture was further purified by prep-SFC to yield tert-butyl (2S,4R*)-4-((1-(3-(2,6-bis(benzyloxy)pyridin-3-yl)-1-methyl-1H-indazol-6-yl)piperidin-4-yl)methyl)-2-methylpiperidine-1-carboxylate (peak-1) and tert-butyl (2S,4S*)-4-((1-(3-(2,6-bis(benzyloxy)pyridin-3-yl)-1-methyl-1H-indazol-6-yl)piperidin-4-yl)methyl)-2-methylpiperidine-1-carboxylate (peak-2). LCMS: 716.5

Step 5a: tert-butyl (2S,4R*)-4-((1-(3-(2,6-dioxopiperidin-3-yl)-1-methyl-1H-indazol-6-yl)piperidin-4-yl)methyl)-2-methylpiperidine-1-carboxylate. To tert-butyl (2S,4R*)-4-((1-(3-(2,6-bis(benzyloxy)pyridin-3-yl)-1-methyl-1H-indazol-6-yl)piperidin-4-yl)methyl)-2-methylpiperidine-1-carboxylate (0.15 mmol) in THE (5 mL) and EtOH (0.5 mL) was added palladium hydroxide on carbon, powder, unreduced, 20% Pd, moisture ca 60% (65 mg), and the reaction apparatus was evacuated and backfilled with H₂ three times. The reaction mixture was stirred under an atmosphere of H₂ (15 psi) at room temperature for 16 hours. The mixture was then filtered through celite. The filtrate was concentrated under reduced pressure to yield the title compound which was used without further purification. LCMS: 538.4

Step 6a: 3-(1-methyl-6-(4-(((2S,4R*)-2-methylpiperidin-4-yl)methyl)piperidin-1-yl)-1H-indazol-3-yl)piperidine-2,6-dione (INT 36-1). To tert-butyl (2S,4R*)-4-((1-(3-(2,6-dioxopiperidin-3-yl)-1-methyl-1H-indazol-6-yl)piperidin-4-yl)methyl)-2-methylpiperidine-1-carboxylate (0.15 mmol) in DCM (3 mL) was added trifluoroacetic acid (0.05 mL). The reaction mixture was stirred at room temperature for 1 h, then diluted with EtOAc and concentrated under reduced pressure to yield the title compound which was used without further purification. LCMS: 438.3

The following intermediates were made in a similar fashion to INT 36-1 and are shown below in Table 2T. To prepare the below intermediates, different reagents/starting materials were used than previously described in the steps toward INT 36-1 and are noted in the last column of Table 2T—“Changes to Procedure: Different Reagents/Starting Materials”. A person of ordinary skill in the art will readily recognize which reagents/starting materials used for the synthesis of INT 36-1 were replaced with the different reagents/starting materials noted below.

TABLE 2T
Intermediates

| | | | Changes to Procedure: Different |
|---|---|---|---|
| Intermediate | Structure | LCMS | Reagents/Starting Materials |
| INT 36-2 | 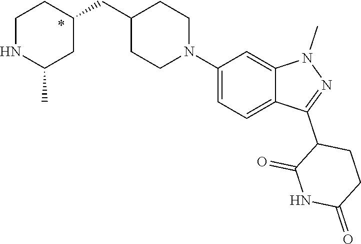 [SMILES: C[C@H]1C[C@@H](CC2CCN(c3ccc4c(C5CCC(=O)NC5=O)nn(C)c4c3)CC2)CCN1] | 438.3 | Peak-2 used in step 5b |
| INT 36-3 | 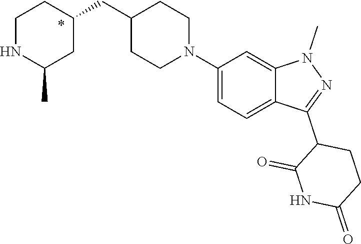 [SMILES: C[C@@H]1C[C@@H](CC2CCN(c3ccc4c(C5CCC(=O)NC5=O)nn(C)c4c3)CC2)CCN1] | 438.3 | (2R)-2-methyl-4-oxo-piperidine-1- carboxylate in step 1. Peak-1 isolated by SFC in step 4 and carried forward. |
| INT 36-4 | 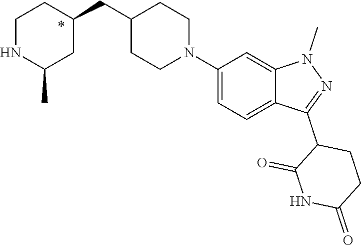 [SMILES: C[C@@H]1C[C@H](CC2CCN(c3ccc4c(C5CCC(=O)NC5=O)nn(C)c4c3)CC2)CCN1] | 438.3 | (2R)-2-methyl-4-oxo-piperidine-1- carboxylate in step 1. Peak-2 isolated by SFC in step 4 and carried forward. |
| INT 36-5 | 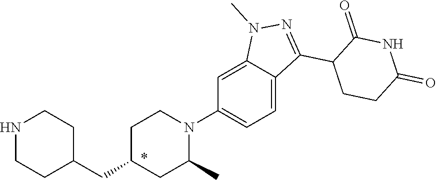 [SMILES: C[C@H]1C[C@H](CC2CCNCC2)CCN1c1ccc2c(C3CCC(=O)NC3=O)nn(C)c2c1] | 438.3 | benzyl (S)-2-methyl-4-oxopiperidine- 1-carboxylate in step 1. tert-butyl 4- bromopiperidine-1-carboxylate in step 2. |
| INT 36-6 | 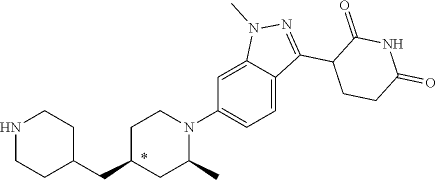 [SMILES: C[C@H]1C[C@@H](CC2CCNCC2)CCN1c1ccc2c(C3CCC(=O)NC3=O)nn(C)c2c1] | 438.3 | benzyl (S)-2-methyl-4-oxopiperidine- 1-carboxylate in step 1. tert-butyl 4- bromopiperidine-1-carboxylate in step 2. |

### Intermediates 37-1-37-6.

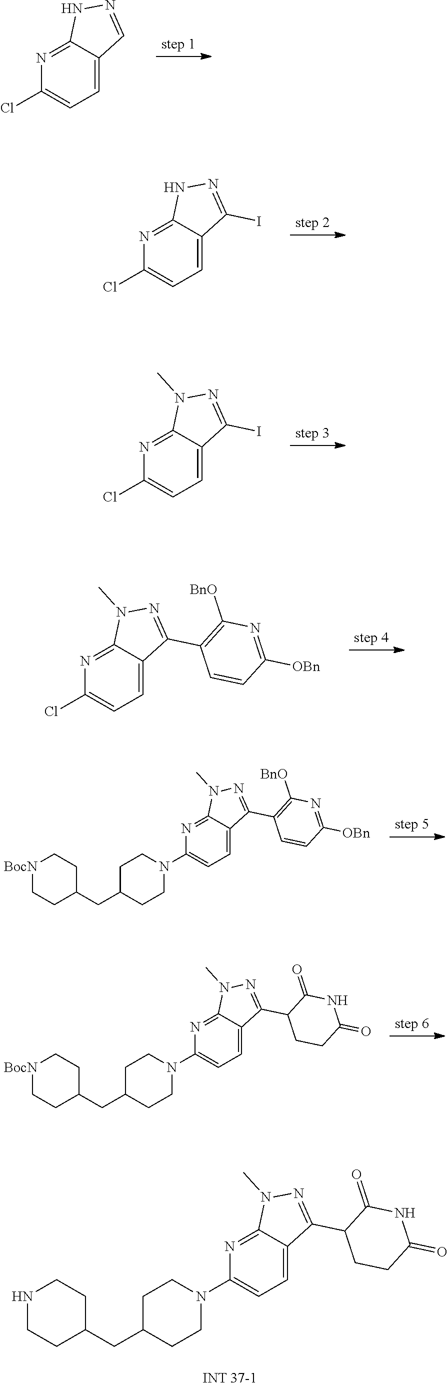

Step 1: 6-chloro-3-iodo-1H-pyrazolo[3,4-b]pyridine. To a solution of 6-chloro-1H-pyrazolo[3,4-b]pyridine (13 mmol) in DMF (20 mL) was added N-iodosuccinimide (16 mmol) at room temperature. The reaction mixture was stirred at 80° C. for 2 h. Upon completion, the solution was poured into 200 mL of water. The precipitate was isolated after filtration and used directly to obtain the title compound without further purification. LCMS: 280.0.

Step 2: 6-chloro-3-iodo-1-methyl-pyrazolo[3,4-b]pyridine. To a solution of 6-chloro-3-iodo-1H-pyrazolo[3,4-b]pyridine (7.2 mmol) and potassium carbonate (22 mmol) in DMF was added iodomethane (14 mmol). The reaction mixture was stirred at room temperature for 16 h. Upon completion, the solution was poured into 200 mL of water. The precipitate was isolated after filtration and used directly to obtain the title compound without further purification. LCMS: 294.1.

Step 3: 6-chloro-3-(2,6-dibenzyloxy-3-pyridyl)-1-methyl-pyrazolo[3,4-b]pyridine. A solution of 6-chloro-3-iodo-1-methyl-pyrazolo[3,4-b]pyridine (3.4 mmol), 2,6-dibenzyloxy-3-(4,4,5,5-tetramethyl-1,3,2-dioxaborolan-2-yl)pyridine (4.1 mmol), 1,1′-bis(diphenylphosphino)ferrocenedichloropalladium(II) (0.17 mmol) and cesium carbonate (10 mmol) in tetrahydrofuran (10 mL) and water (1 mL) was heated at 100° C. under argon for 16 h. The mixture was then cooled to room temperature, filtered through Celite and concentrated under reduced pressure. The residue was purified by flash column chromatography on silica gel (0% to 20% ethyl acetate in hexanes) to afford the title compound. LCMS: 457.3.

Step 4: tert-butyl 4-[[1-[3-(2,6-dibenzyloxy-3-pyridyl)-1-methyl-pyrazolo[3,4-b]pyridin-6-yl]-4-piperidyl]methyl]piperidine-1-carboxylate. A solution of 6-chloro-3-(2,6-dibenzyloxy-3-pyridyl)-1-methyl-pyrazolo[3,4-b]pyridine (1.6 mmol), tert-butyl 4-(4-piperidylmethyl)piperidine-1-carboxylate (2.43 mmol), tris(dibenzylideneacetone)dipalladium(0) (0.049 mmol), RuPhos (0.081 mmol), sodium tert-butoxide (6.5 mmol) in 1,4-dioxane (8 mL) was heated under argon at 100° C. for 16 h. Upon completion, the reaction was cooled to room temperature, then saturated ammonium chloride solution was added. The mixture was extracted with ethyl acetate (3×25 mL), the combined organic layers were dried over anhydrous sodium sulfate, filtered and concentrated under reduced pressure. The residue was purified by flash column chromatography on silica gel (0% to 30% ethyl acetate in hexanes). Fractions containing the product were pooled and concentrated under reduced pressure to yield the title compound. LCMS: 703.5.

Step 5: tert-butyl 4-[[1-[3-(2,6-dioxo-3-piperidyl)-1-methyl-pyrazolo[3,4-b]pyridin-6-yl]-4-piperidyl]methyl]piperidine-1-carboxylate. A mixture of palladium hydroxide on carbon (4.6 mmol) and tert-butyl 4-[[1-[3-(2,6-dibenzyloxy-3-pyridyl)-1-methyl-pyrazolo[3,4-b]pyridin-6-yl]-4-piperidyl]methyl]piperidine-1-carboxylate (0.93 mmol) in tetrahydrofuran (7 mL) was evacuated under vacuum and backfilled with hydrogen for three times. The reaction mixture was vigorously stirred at room temperature for 12 h. Upon completion, the solution was filtered through a short pad of Celite, washed with dichloromethane (3×10 mL), concentrated under reduced pressure, and used for the next step without further purification. LCMS: 525.4.

Step 6: 3-[1-methyl-6-[4-(4-piperidylmethyl)-1-piperidyl]pyrazolo[3,4-b]pyridin-3-yl]piperidine-2,6-dione (INT 37-1). Trifluoroacetic acid (0.5 mL) was added into a solution of tert-butyl 4-[[1-[3-(2,6-dioxo-3-piperidyl)-1-methyl-pyrazolo[3,4-b]pyridin-6-yl]-4-piperidyl]methyl]piperidine-1-carboxylate (0.88 mmol) in dichloromethane (5 mL) at room temperature. The resulting solution was stirred for 2 h. Upon completion, the solution was concentrated under reduced pressure to afford the title compound which was taken forward without further purification. LCMS: 425.3.

The following intermediates were made in a similar fashion to INT 37-1 and are shown below in Table 2U. To prepare the below intermediates, different reagents/starting materials were used than previously described in the steps toward INT 37-1 and are noted in the last column of Table 2U—“Changes to Procedure: Different Reagents/Starting Materials”. A person of ordinary skill in the art will readily recognize which reagents/starting materials used for the synthesis of INT 37-1 were replaced with the different reagents/starting materials noted below.

TABLE 2U
Intermediates

| | | | Changes to Procedure: Different |
|---|---|---|---|
| Intermediate | Structure | LCMS | Reagents/Starting Materials |
| INT 37-2 | 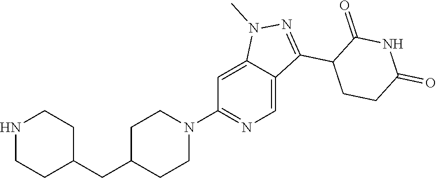 [SMILES: Cn1nc(C2CCC(=O)NC2=O)c2cnc(N3CCC(CC4CCNCC4)CC3)cc21] | 425.3 | 6-chloro-1H-pyrazolo[4,3-c]pyridine in step 1 |
| INT 37-3 | 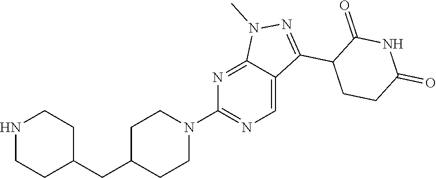 [SMILES: Cn1nc(C2CCC(=O)NC2=O)c2cnc(N3CCC(CC4CCNCC4)CC3)nc21] | 426.3 | 6-chloro-1H-pyrazolo[3,4-d]pyrimidine in step 1 |
| INT 37-4 | 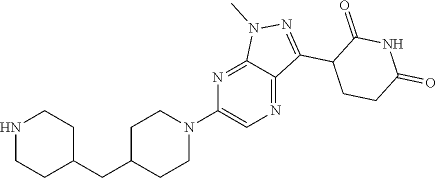 [SMILES: Cn1nc(C2CCC(=O)NC2=O)c2ncc(N3CCC(CC4CCNCC4)CC3)nc21] | 426.3 | 6-chloro-1H-pyrazolo[3,4-b]pyrazine in step 1 |
| INT 37-5 | 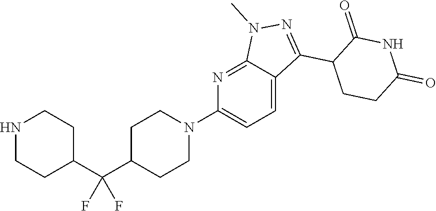 [SMILES: Cn1nc(C2CCC(=O)NC2=O)c2ccc(N3CCC(C(F)(F)C4CCNCC4)CC3)nc21] | 461.3 | tert-butyl 4-(difluoro(piperidin-4- yl)methyl)piperidine-1-carboxylate in step 4. |
| INT 37-6 | 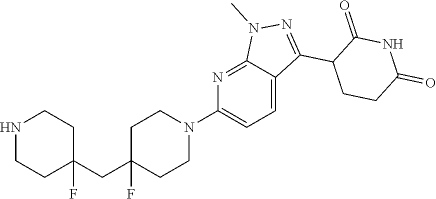 [SMILES: Cn1nc(C2CCC(=O)NC2=O)c2ccc(N3CCC(F)(CC4(F)CCNCC4)CC3)nc21] | 461.3 | tert-butyl 4-fluoro-4-((4- fluoropiperidin-4-y1)methyl)piperidine- 1-carboxylate in step 4 |

### Intermediates 38-1-38-5.

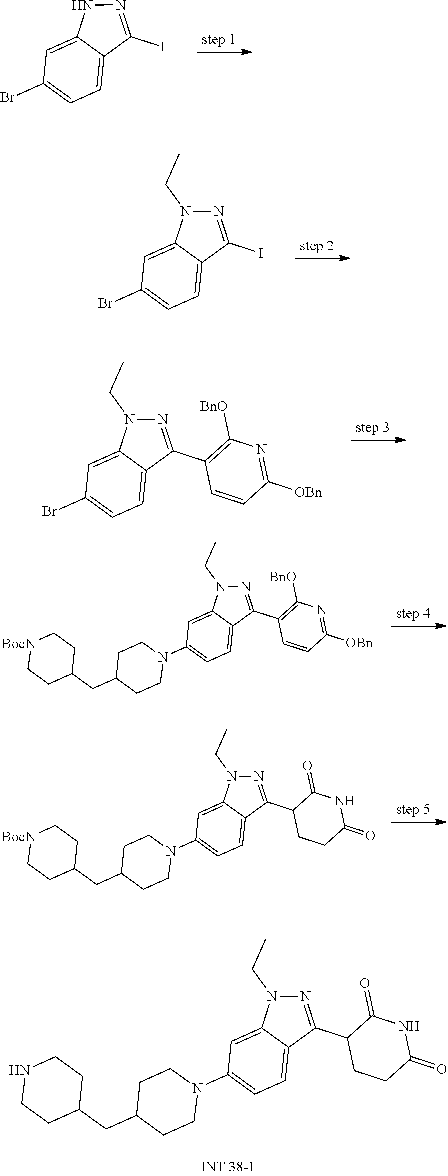 [SMILES: Brc1ccc2c(I)n[nH]c2c1 ; CCn1nc(I)c2ccc(Br)cc21 ; CCn1nc(-c2ccc(OCc3ccccc3)nc2OCc2ccccc2)c2ccc(Br)cc21 ; CCn1nc(-c2ccc(OCc3ccccc3)nc2OCc2ccccc2)c2ccc(N3CCC(CC4CCN(C(=O)OC(C)(C)C)CC4)CC3)cc21 ; CCn1nc(C2CCC(=O)NC2=O)c2ccc(N3CCC(CC4CCN(C(=O)OC(C)(C)C)CC4)CC3)cc21 ; CCn1nc(C2CCC(=O)NC2=O)c2ccc(N3CCC(CC4CCNCC4)CC3)cc21]

Step 1: 6-bromo-1-ethyl-3-iodo-1H-indazole. To a stirred solution of 6-bromo-3-iodo-1H-indazole (17.3 mmol) in DMF (46 mL) was added potassium hydroxide (26.0 mmol) at 0° C. After stirring for 5 minutes, diethyl sulfate (20.8 mmol) was added dropwise. The reaction mixture was stirred at 0° C. for 1 hour under nitrogen. The mixture was then quenched with water (100 mL) and extracted with ethyl acetate. The combined organic layers were dried over anhydrous sodium sulfate, filtered, and concentrated under reduced pressure. The residue was purified by flash column chromatography on silica gel to afford the title compound. LCMS: 351.1.

Step 2: 3-(2,6-bis(benzyloxy)pyridin-3-yl)-6-bromo-1-ethyl-1H-indazole. To a stirred solution of 6-bromo-1-ethyl-3-iodo-1H-indazole (8.8 mmol) and 2,6-bis(benzyloxy)-3-(4,4,5,5-tetramethyl-1,3,2-dioxaborolan-2-yl)pyridine (8.8 mmol) in 1,4-dioxane (50 mL), was added cesium carbonate (22 mmol) and water (10 mL) at room temperature. The resulting reaction mixture was degassed with argon for 15 minutes. [1,1′-bis(diphenylphosphino)ferrocene]dichloropalladium(II) (0.88 mmol) was added to the reaction mixture under argon and was degassed for another 5 minutes. The reaction mixture was heated to 95° C. and stirred for 16 hours. After completion, the reaction mixture was cooled to room temperature, filtered through a pad of Celite, and washed with ethyl acetate. The combined filtrate was washed with water, dried over sodium sulfate and concentrated under reduced pressure. The residue was purified by flash column chromatography on silica gel eluting with ethyl acetate/hexanes to afford the title compound. LCMS: 515.8.

Step 3: tert-butyl 4-((1-(3-(2,6-bis(benzyloxy)pyridin-3-yl)-1-ethyl-1H-indazol-6-yl)piperidin-4-yl)methyl)piperidine-1-carboxylate. A vigorously stirred solution of tert-butyl 4-(piperidin-4-ylmethyl)piperidine-1-carboxylate (0.8 mmol), 3-(2,6-bis(benzyloxy)pyridin-3-yl)-6-bromo-1-ethyl-1H-indazole (0.6 mmol), tris(dibenzylideneacetone)dipalladium(0) (0.012 mmol), 2-dicyclohexylphosphino-2′,4′,6′-triisopropylbiphenyl (0.018 mmol), cesium carbonate (1.8 mmol) in 1,4-dioxane (6 mL) was heated under argon at 100° C. for 16 hours. The reaction mixture was filtered through a short pad of Celite and washed with dichloromethane (3×10 mL), then concentrated under reduced pressure. The residue was purified by flash column chromatography on silica gel (100% ethyl acetate) to afford the title compound. LCMS: 716.5.

Step 4: tert-butyl 4-((1-(3-(2,6-dioxopiperidin-3-yl)-1-ethyl-1H-indazol-6-yl)piperidin-4-yl)methyl)piperidine-1-carboxylate. A mixture of palladium hydroxide on carbon (4.7 mmol, 20% wt Pd) and tert-butyl 4-((1-(3-(2,6-bis(benzyloxy)pyridin-3-yl)-1-ethyl-1H-indazol-6-yl)piperidin-4-yl)methyl)piperidine-1-carboxylate (0.94 mmol) in THE (10 mL) and EtOH (2 mL) was evacuated under vacuum and backfilled with hydrogen three times. The reaction mixture was vigorously stirred at room temperature for 12 hours. Upon completion, the solution was filtered through a short pad of Celite, washed with dichloromethane (3×10 mL), then concentrated under reduced pressure. The residue was purified by flash column chromatography on silica gel (0% to 50% ethyl acetate in hexanes) to afford the title compound. LCMS: 538.4.

Step 5: 3-(1-methyl-6-(4-(piperazin-1-ylmethyl)piperidin-1-yl)-1H-indazol-3-yl)piperidine-2,6-dione (INT 38-1). Trifluoroacetic acid (1.0 mL) was added into a solution of 4-((1-(3-(2,6-dioxopiperidin-3-yl)-1-ethyl-1H-indazol-6-yl)piperidin-4-yl)methyl)piperidine-1-carboxylate (0.8 mmol) in dichloromethane (10 mL) at room temperature. The resulting solution was stirred at room temperature for 2 hours. Upon completion, the solution was concentrated under reduced pressure and used for the next step without further purification. LCMS: 438.3.

The following intermediates were made in a similar fashion to INT 38-1 and are shown below in Table 2V. To prepare the below intermediates, different reagents/starting materials were used than previously described in the steps toward INT 38-1 and are noted in the last column of Table 2V—“Changes to Procedure: Different Reagents/Starting Materials”. A person of ordinary skill in the art will readily recognize which reagents/starting materials used for the synthesis of INT 38-1 were replaced with the different reagents/starting materials noted below.

TABLE 2V
Intermediates

| | | | Changes to Procedure: Different |
|---|---|---|---|
| Intermediate | Structure | LCMS | Reagents/Starting Materials |
| 38-2 | 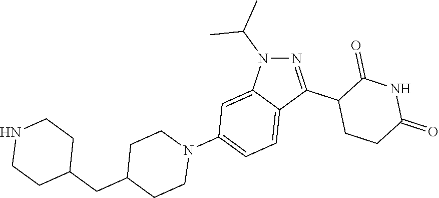 [SMILES: CC(C)n1nc(C2CCC(=O)NC2=O)c2ccc(N3CCC(CC4CCNCC4)CC3)cc21] | 452.4 | 2-iodopropane (2.0 equiv), potassium carbonate (4 equiv), MeCN (180 mL), 90° C. in step 1. |
| 38-3 | 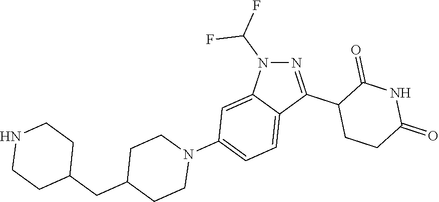 [SMILES: O=C1CCC(c2nn(C(F)F)c3cc(N4CCC(CC5CCNCC5)CC4)ccc23)C(=O)N1] | 460.3 | sodium 2-chloro-2,2-difluoroacetate (3 equiv), potassium carbonate (6 equiv), DMF/water (4:1, 0.2 M), 120° C. in step 1. |
| 38-4 | 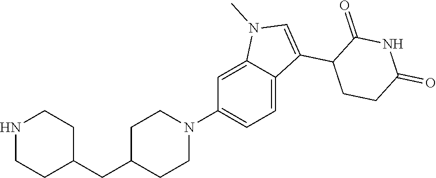 [SMILES: Cn1cc(C2CCC(=O)NC2=O)c2ccc(N3CCC(CC4CCNCC4)CC3)cc21] | 423.4 | Step 1 omitted. 6-bromo-3-iodo-1- methyl-indole in step 2. |
| 38-5 | 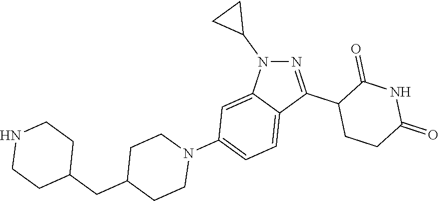 [SMILES: O=C1CCC(c2nn(C3CC3)c3cc(N4CCC(CC5CCNCC5)CC4)ccc23)C(=O)N1] | 450.3 | A mixture of 6-bromo-3-iodo-1H- indazole (31.0 mmol), cyclopropylboronic acid (93 mmol), copper (II) acetate (93 mmol), and pyridine (619 mmol) in DMF (100mL) was stirred at 80° C. under air for 12 h. Upon completion, the reaction mixture was concentrated and purified by silica gel chromatography to afford 6-bromo- 1-cyclopropyl-3-iodo-indazole (LCMS: 526.2), which was used in subsequent steps. |

### Intermediates 39 1A-B-40 1A-B.

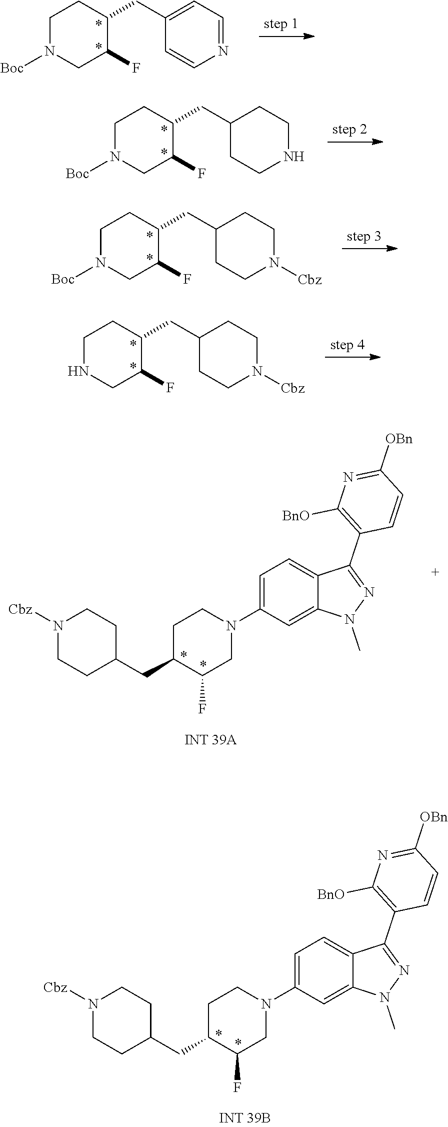

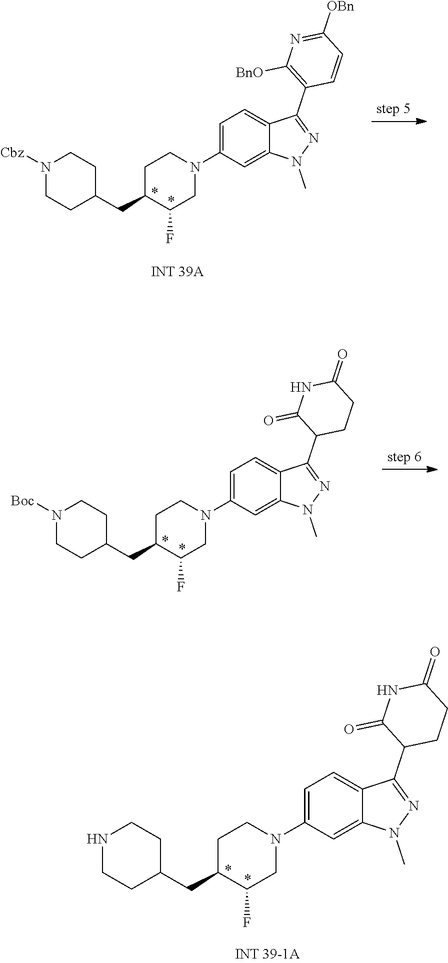 [SMILES: Cn1nc(-c2ccc(OCc3ccccc3)nc2OCc2ccccc2)c2ccc(N3CC[C@H](CC4CCN(C(=O)OCc5ccccc5)CC4)[C@@H](F)C3)cc21 ; Cn1nc(C2CCC(=O)NC2=O)c2ccc(N3CC[C@H](CC4CCN(C(=O)OC(C)(C)C)CC4)[C@@H](F)C3)cc21 ; Cn1nc(C2CCC(=O)NC2=O)c2ccc(N3CC[C@H](CC4CCNCC4)[C@@H](F)C3)cc21]

Step 1: racemic tert-butyl-3-fluoro-4-(piperidin-4-ylmethyl)piperidine-1-carboxylate. To a stirred solution of tert-butyl (3R*,4S*)-3-fluoro-4-(pyridin-4-ylmethyl)piperidine-1-carboxylate (4.0 g) in EtOH (40 mL) was added acetic acid (1.18 mL) dropwise followed by platinum (IV) oxide (0.46 g). The reaction mixture was then stirred at room temperature for 24 h under a hydrogen balloon. Upon completion, the reaction mixture was filtered over a celite pad, washed multiple times with ethanol and concentrated to yield the title compound which was used without further purification. LCMS: 300.0

Step 2: racemic tert-butyl-4-((1-((benzyloxy)carbonyl)piperidin-4-yl)methyl)-3-fluoropiperidine-1-carboxylate. To a stirred solution of racemic tert-butyl-3-fluoro-4-(piperidin-4-ylmethyl)piperidine-1-carboxylate (3.5 g) in THE (40 mL) water (10 mL). Sodium bicarbonate (2.4 g) was added. Benzyl chloroformate (1.6 mL) was added, then the reaction mixture was stirred at rt for 3 h. Water was then added, and the mixture was extracted with ethyl acetate. The organic layers were combined, washed with brine, dried over anhydrous sodium sulfate, filtered, and concentrated under reduced pressure to afford the title compound. LCMS: 435.3.

Step 3: racemic benzyl 4-(((3R,4S)-3-fluoropiperidin-4-yl)methyl)piperidine-1-carboxylate. To a stirred solution of racemic tert-butyl-4-((1-((benzyloxy)carbonyl)piperidin-4-yl)methyl)-3-fluoropiperidine-1-carboxylate (1.5 g) in dichloromethane (15 mL) was added hydrogen chloride 4.0 M in 1,4-dioxane (5.1 mL) at 0° C. The reaction mixture was stirred at room temperature for 3 h. Upon completion, the mixture was concentrated under reduced pressure and the residue was triturated with diethyl ether to afford the title compound. LCMS: 335.2

Step 4: Benzyl 4-(((3R,4S)-1-(3-(2,6-bis(benzyloxy)pyridin-3-yl)-1-methyl-1H-indazol-6-yl)-3-fluoropiperidin-4-yl)methyl)piperidine-1-carboxylate (INT 39A). To a stirred degassed solution of racemic benzyl 4-(((3R,4S)-3-fluoropiperidin-4-yl)methyl)piperidine-1-carboxylate (1.1) and 3-(2,6-bis(benzyloxy)pyridin-3-yl)-6-bromo-1-methyl-1H-indazole (1.6 g) in 1,4-dioxane (10 mL) was added cesium carbonate (3.2 g), XPhos (0.15 g) and tris(dibenzylideneacetone)dipalladium(0) (0.15 g). The mixture was then degassed with nitrogen and subsequently stirred at 100° C., under argon, for 16 hours. The reaction mixture was then cooled to room temperature, filtered, and concentrated under reduced pressure to give a crude residue. The residue was then purified by flash column chromatography on silica gel eluting with ethyl acetate and hexanes. The residue was subsequently further separated by chiral SFC (Chiralpak SC 250×30×5 μm, 60% CO₂/40% ACN:IPA (1:1) with 0.5% isopropylamine, 100 g/min) into two isomers, arbitrarily labeled as INT 39A (benzyl 4-(((3R*,4S*)-1-(3-(2,6-bis(benzyloxy)pyridin-3-yl)-1-methyl-1H-indazol-6-yl)-3-fluoropiperidin-4-yl)methyl)piperidine-1-carboxylate). INT 39B (benzyl 4-(((3S*,4R*)-1-(3-(2,6-bis(benzyloxy)pyridin-3-yl)-1-methyl-1H-indazol-6-yl)-3-fluoropiperidin-4-yl)methyl)piperidine-1-carboxylate). Relative stereochemistry was established by the trans starting material in step 1.

Step 5: tert-butyl 4-(((3R*,4S*)-1-(3-(2,6-dioxopiperidin-3-yl)-1-methyl-1H-indazol-6-yl)-3-fluoropiperidin-4-yl)methyl)piperidine-1-carboxylate. To a stirred solution of INT 39A (0.4 g) in tetrahydrofuran (5 mL) were added palladium hydroxide on carbon (0.35 g, 20 wt % Pd) and palladium on activated carbon (0.35 g, 10 wt % Pd). Di-tert-butyl dicarbonate (0.36 mL) was then added, and the reaction mixture was stirred under a hydrogen atmosphere (1 amt) at room temperature for 16 hours. Upon completion, the mixture was filtered through a short pad of Celite, washed with ethyl acetate. The combined filtrate was concentrated under reduced pressure to afford a crude residue. The residue was purified by flash column chromatography on silica gel, eluting with 60-70% ethyl acetate in petroleum ether, to afford the title compound. LCMS: 542.4. Relative stereochemistry was established by the trans starting material in step 1.

Step 6: 3-(6-((3R*,4S*)-3-fluoro-4-(piperidin-4-ylmethyl)piperidin-1-yl)-1-methyl-1H-indazol-3-yl)piperidine-2,6-dione (INT 39-1A). tert-butyl 4-(((3R*,4S*)-1-(3-(2,6-dioxopiperidin-3-yl)-1-methyl-1H-indazol-6-yl)-3-fluoropiperidin-4-yl)methyl)piperidine-1-carboxylate (0.22 g) was dissolved in DCM (10 mL), then trifluoroacetic acid (2 mL) was added dropwise at room temperature. The reaction mixture was stirred at room temperature for 3 h, then concentrated under reduced pressure to yield the title compound which was used without further purification. LCMS: 442.3. Relative stereochemistry was established by the trans starting material in step 1.

The following intermediates were made in a similar fashion to INT 39-1A and are shown below in Table 2V. To prepare the below intermediates, different reagents/starting materials were used than previously described in the steps toward INT 39-1A and are noted in the last column of Table 2V—“Changes to Procedure: Different Reagents/Starting Materials”. A person of ordinary skill in the art will readily recognize which reagents/starting materials used for the synthesis of INT 39-1A were replaced with the different reagents/starting materials noted below.

TABLE 2W
Intermediates

| | | | Changes to Procedure: Different |
|---|---|---|---|
| Intermediate | Structure | LCMS | Reagents/Starting Materials |
| 39-1B | 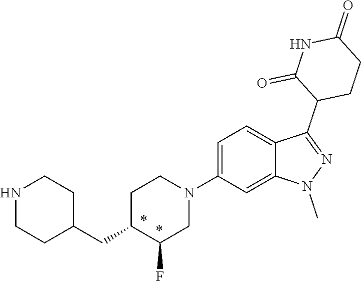 [SMILES: Cn1nc(C2CCC(=O)NC2=O)c2ccc(N3CC[C@@H](CC4CCNCC4)[C@H](F)C3)cc21] | 442.3 | INT 39B in step 5 |
| 40-1A | 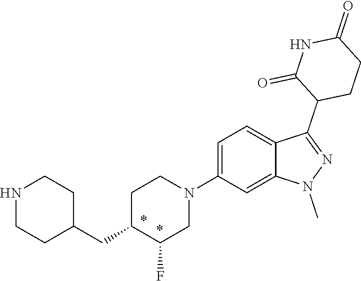 [SMILES: Cn1nc(C2CCC(=O)NC2=O)c2ccc(N3CC[C@@H](CC4CCNCC4)[C@@H](F)C3)cc21] | 442.3 | racemic cis tert-butyl (3R,4R)-3-fluoro- 4-(pyridin-4-ylmethyl) piperidine-1- carboxylate tert-butyl 3-methyl-4- oxopiperidine-1-carboxylate in step 1. |
| 40-1B | 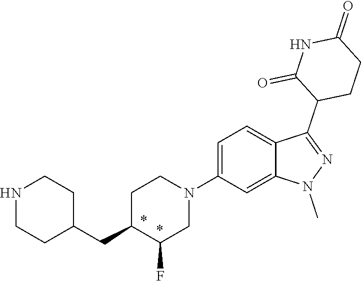 [SMILES: Cn1nc(C2CCC(=O)NC2=O)c2ccc(N3CC[C@H](CC4CCNCC4)[C@H](F)C3)cc21] | 442.3 | racemic cis tert-butyl (3R,4R)-3-fluoro- 4-(pyridin-4-ylmethyl) piperidine-1- carboxylate tert-butyl 3-methyl-4- oxopiperidine-1-carboxylate in step 1. |

### Intermediates 41-1A-B-42-1A-D.

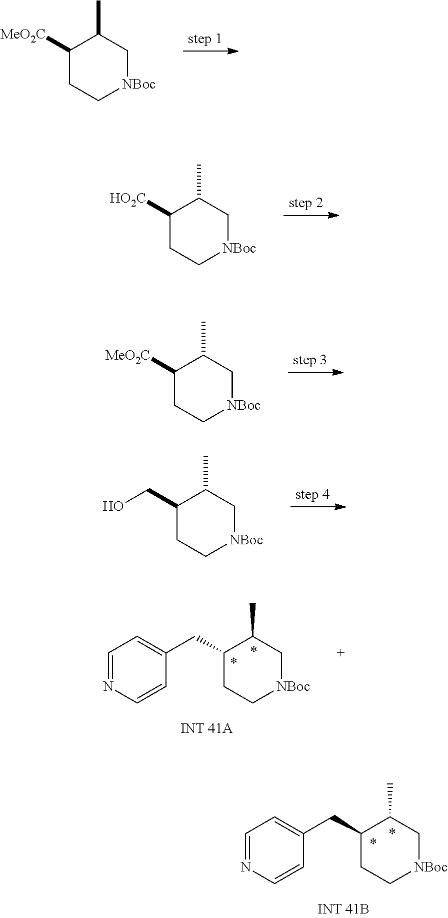

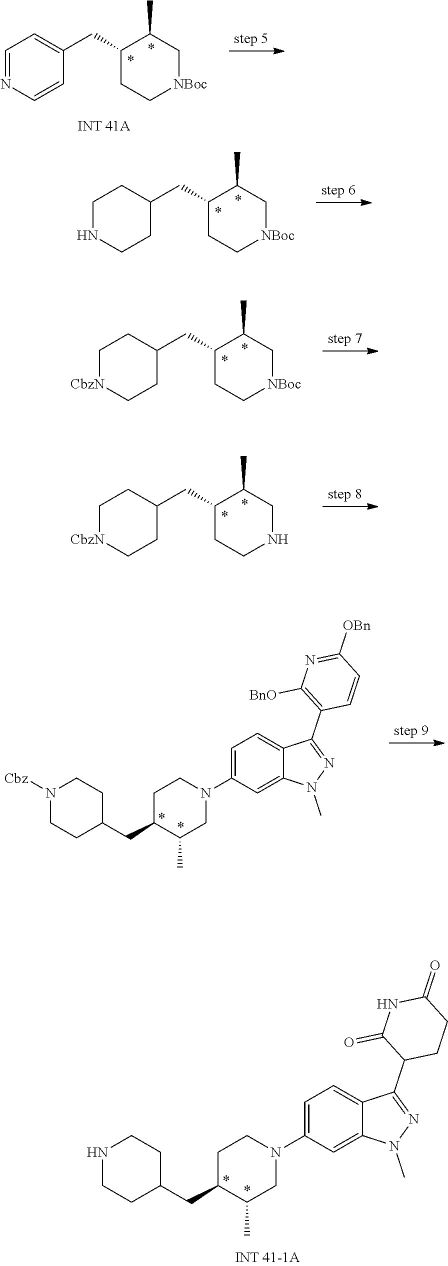 [SMILES: C[C@H]1CN(C(=O)OC(C)(C)C)CC[C@@H]1Cc1ccncc1 ; C[C@H]1CN(C(=O)OC(C)(C)C)CC[C@@H]1CC1CCNCC1 ; C[C@H]1CN(C(=O)OC(C)(C)C)CC[C@@H]1CC1CCN(C(=O)OCc2ccccc2)CC1 ; C[C@H]1CNCC[C@@H]1CC1CCN(C(=O)OCc2ccccc2)CC1 ; C[C@H]1CN(c2ccc3c(-c4ccc(OCc5ccccc5)nc4OCc4ccccc4)nn(C)c3c2)CC[C@@H]1CC1CCN(C(=O)OCc2ccccc2)CC1 ; C[C@H]1CN(c2ccc3c(C4CCC(=O)NC4=O)nn(C)c3c2)CC[C@@H]1CC1CCNCC1]

Step 1: 1-(tert-butoxycarbonyl)-3-methylpiperidine-4-carboxylic acid. Anhydrous methanol (500 mL) was treated portion-wise with sodium (8.9 g) and stirred at 25° C. for 1 h under nitrogen. A cis-diastereomeric mixture of 1-(tert-butyl) 4-methyl 3-methylpiperidine-1,4-dicarboxylate (10 g) was then added, and the reaction mixture was stirred at 70° C. for 12 h under nitrogen. After completion, the mixture was diluted with water, cooled to 0° C., and the pH was adjusted to 5-6 using citric acid. The aqueous layer was extracted with ethyl acetate, and the combined organic layers were dried over Na₂SO₄, filtered, and concentrated under reduced pressure to afford the title compound, which was used in the subsequent step without further purification. LCMS: 188.2

Step 2: trans-1-tert-butyl 4-methyl 3-methylpiperidine-1,4-dicarboxylate. To a solution of 1-tert-butoxycarbonyl-3-methylpiperidine-4-carboxylic acid (9.4 g, 38.6 mmol) in DMF (50 mL), potassium carbonate (10.7 g, 77.3 mmol) and iodomethane (8.23 g, 58.0 mmol) were added at 0° C. The mixture was stirred at 25° C. for 12 h. Upon completion, the reaction mixture was filtered, and the filtrate was extracted with water and ethyl acetate. Combined organic layers were dried over Na₂SO₄, filtered, and concentrated under reduced pressure to yield a crude residue, which was purified by flash silica gel chromatography eluting with 0-20% EtOAc in hexanes. The residue was then further separated via SFC (column: Daicel Chiralpak IC, 250 mm×50 mm, 10 μm; mobile phase: A=heptane, B=IPA; B %=15.00%, 4.00 min). Peak 1 was isolated as trans-1-tert-butyl 4-methyl 3-methylpiperidine-1,4-dicarboxylate. LCMS: 202.1. Peak 2 was isolated as cis-1-tert-butyl 4-methyl 3-methylpiperidine-1,4-dicarboxylate. LCMS: 202.1.

Step 3: tert-butyl-4-(hydroxymethyl)-3-methylpiperidine-1-carboxylate. To a mixture of trans-1-tert-butyl 4-methyl 3-methylpiperidine-1,4-dicarboxylate (4.60 g, 17.9 mmol) in THE (50.0 mL) was added a solution of LAH in THE (2.5 M, 17.9 mL) dropwise at 0° C. The mixture was stirred at 25° C. for 2 h under nitrogen. Upon completion, the reaction was quenched by the addition of sodium sulfate decahydrate at 0° C. The mixture was filtered, and the filtrate was concentrated under reduced pressure to afford the tile compound, which was used without purification. LCMS: 174.1.

Step 4: tert-butyl (3R,4S)-3-methyl-4-(pyridin-4-ylmethyl)piperidine-1-carboxylate and tert-butyl (3S,4R)-3-methyl-4-(pyridin-4-ylmethyl)piperidine-1-carboxylate. To a solution of a mixture of trans-tert-butyl-4-(hydroxymethyl)-3-methylpiperidine-1-carboxylate diastereomers (1.4 g, 6.2 mmol) in methyl tert-butyl ether (5 mL) was added deoxazole (2.2 g, 5.6 mmol) at 25° C. under nitrogen. The mixture was stirred at 25° C. for 5 min. Pyridine (0.4 g, 5.6 mmol) in methyl tert-butyl ether (5 mL) was then added dropwise at 25° C. under nitrogen, and the mixture was stirred for an additional 10 min. The reaction mixture was filtered, and the filtrate was added to a mixture containing bis[2-(2-pyridyl)phenyl]iridium(I) 4-tert-butyl-2-(4-tert-butyl-2-pyridyl)pyridine hexafluorophosphate (48 mg, 0.05 mmol), 4-tert-butyl-2-(4-tert-butyl-2-pyridyl)pyridine dibromonickel (86 mg, 0.17 mmol), quinuclidine (0.690 g, 6.2 mmol), and 4-bromopyridine (0.560 g, 3.54 mmol) in N,N-dimethylacetamide (10 mL). The mixture was stirred at 25° C. for 12 h under argon and irradiated with blue LEDs (450 nm). Upon completion, the reaction mixture was diluted with water and extracted with ethyl acetate. The combined organic layers were washed with brine, dried over Na₂SO₄, filtered, and concentrated under reduced pressure to afford a crude residue, which was purified by flash silica gel chromatography eluting with 0-20% EtOAc in hexanes. The residue was then further separated via SFC (column: Daicel Chiralpak AD; mobile phase A=CO₂, B=33% MeOH with 0.1% NH₃·H₂O), yielding two isomers arbitrarily labeled as INT 41A (peak 1) and INT 41B (peak 2). INT 41A: tert-butyl (3R*,4S*)-3-methyl-4-(pyridin-4-ylmethyl)piperidine-1-carboxylate. LCMS: 291.2 INT 41B: tert-butyl (3S*,4R*)-3-methyl-4-(pyridin-4-ylmethyl)piperidine-1-carboxylate. LCMS: 291.2. Relative stereochemistry was established by the trans starting material in step 4.

Step 5: (3R*,4S*)-tert-butyl 3-methyl-4-(piperidin-4-ylmethyl)piperidine-1-carboxylate. To a mixture of (3R*,4S*)-tert-butyl 3-methyl-4-(pyridin-4-ylmethyl)piperidine-1-carboxylate (0.55 g, 1.9 mmol) in methanol (10 mL) was added palladium on carbon (0.60 g) under argon. The mixture was degassed and purged with hydrogen three times, then stirred at 70° C. for 12 h under hydrogen (50 psi). Upon completion, the reaction mixture was filtered, and the filtrate was concentrated under reduced pressure to yield the title compound which was used without further purification. LCMS: 297.2. Relative stereochemistry was established by the trans starting material in step 4.

Step 6: tert-butyl (3R*,4S*)-4-((1-((benzyloxy)carbonyl)piperidin-4-yl)methyl)-3-methylpiperidine-1-carboxylate. To a mixture of (3R*,4S*)-tert-butyl 3-methyl-4-(piperidin-4-ylmethyl)piperidine-1-carboxylate (0.45 g, 1.5 mmol) in dichloromethane (5.0 mL) was added benzyl chloroformate (0.31 g, 1.8 mmol) and triethylamine (0.20 g, 2.0 mmol) at 0° C. The mixture was stirred at 25° C. for 2 h. Upon completion, the reaction mixture was concentrated under reduced pressure. The residue was diluted with water and extracted with dichloromethane. The combined organic layers were washed with brine, dried over Na₂SO₄, filtered, and concentrated under reduced pressure to afford a crude residue. The residue was purified by flash silica gel chromatography eluting with 0-30% EtOAc in hexanes. Fractions containing the product were pooled and concentrated under reduced pressure to yield the title compound. LCMS: 331.2. Relative stereochemistry was established by the trans starting material in step 4.

Step 7: Benzyl 4-(((3R*,4S*)-3-methylpiperidin-4-yl)methyl)piperidine-1-carboxylate. To a mixture of (3R*,4S*)-tert-butyl 4-((1-((benzyloxy)carbonyl)piperidin-4-yl)methyl)-3-methylpiperidine-1-carboxylate (0.45 g, 1.1 mmol) in ethyl acetate (3.0 mL) was added hydrogen chloride (1.0 mL), and the mixture was stirred at 25° C. for 1 h. Upon completion, the reaction was concentrated and purified by RP-HPLC. Fractions containing the product were pooled and lyophilized to yield the title compound. ¹H NMR (400 MHz, CD₃OD) δ ppm 7.40-7.26 (m, 5H), 5.10 (s, 2H), 4.12 (br d, 2H, J=12.8 Hz), 3.37 (br d, 1H, J=12.4 Hz), 3.25 (br dd, 1H, J=2.8, 12.4 Hz), 2.94 (br t, 1H, J=12.4 Hz), 2.82 (br s, 2H), 2.64 (br t, 1H, J=12.4 Hz), 2.04-2.01 (m, 1H), 1.77 (br d, 1H, J=12.8 Hz), 1.64-1.48 (m, 4H), 1.40-1.30 (m, 2H), 1.19-1.10 (m, 1H), 1.09-0.98 (m, 2H), 0.97 (br d, 3H, J=6.4 Hz). LCMS: 331.2. Relative stereochemistry was established by the trans starting material in step 4.

Step 8: benzyl 4-(((3R*,4S*)-1-(3-(2,6-bis(benzyloxy)pyridin-3-yl)-1-methyl-1H-indazol-6-yl)-3-methylpiperidin-4-yl)methyl)piperidine-1-carboxylate. To a vigorously stirred solution of benzyl 4-(((3R*,4S)*-3-methylpiperidin-4-yl)methyl)piperidine-1-carboxylate (1.2 mmol) in 1,4-dioxane (10 mL) were added 6-bromo-3-(2,6-dibenzyloxy-3-pyridyl)-1-methyl-indazole (1.2 mmol), tris(dibenzylideneacetone)dipalladium(0) (0.036 mmol), XPhos (0.060 mmol), and cesium carbonate (3.6 mmol) and the reaction mixture was heated under argon at 100° C. for 16 h. Upon completion, the reaction mixture was filtered through a short pad of Celite and washed with dichloromethane, then concentrated under reduced pressure. The residue was purified by flash column chromatography on silica gel (100% ethyl acetate) to afford the title compound. LCMS: 750.5. Relative stereochemistry was established by the trans starting material in step 4.

Step 9: 3-(1-methyl-6-((3R*,4S*)-3-methyl-4-(piperidin-4-ylmethyl)piperidin-1-yl)-1H-indazol-3-yl)piperidine-2,6-dione (INT 41-1A). A vigorously stirred solution of palladium hydroxide on carbon (5.7 mmol) and benzyl 4-(((3R*,4S*)-1-(3-(2,6-bis(benzyloxy)pyridin-3-yl)-1-methyl-1H-indazol-6-yl)-3-methylpiperidin-4-yl)methyl)piperidine-1-carboxylate (1.2 mmol) in tetrahydrofuran (5 mL) and ethanol (3 mL) was evacuated under vacuum and backfilled with hydrogen three times. The reaction mixture was stirred vigorously at room temperature for 12 h. Upon completion, the solution was filtered through a short pad of Celite, washed with dichloromethane (3×10 mL), and concentrated under reduced pressure. The residue was purified by flash column chromatography on silica gel (0-50% ethyl acetate in hexanes) to afford the title compound. LCMS: 483.3. Relative stereochemistry was established by the trans starting material in step 4.

The following intermediates were made in a similar fashion to INT 41-1A and are shown below in Table 2X. To prepare the below intermediates, different reagents/starting materials were used than previously described in the steps toward INT 41-1A and are noted in the last column of Table 2X—“Changes to Procedure: Different Reagents/Starting Materials”. A person of ordinary skill in the art will readily recognize which reagents/starting materials used for the synthesis of INT 41-1A were replaced with the different reagents/starting materials noted below.

TABLE 2X
Intermediates

| | | | Changes to Procedure: Different |
|---|---|---|---|
| Intermediate | Structure | LCMS | Reagents/Starting Materials |
| INT 41-1B | 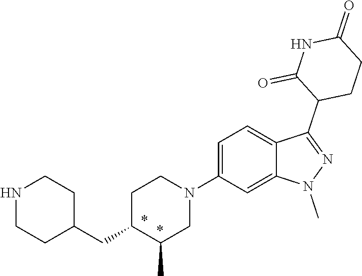 [SMILES: C[C@@H]1CN(c2ccc3c(C4CCC(=O)NC4=O)nn(C)c3c2)CC[C@H]1CC1CCNCC1] | 438.3 | INT 41B used in step 5. |
| INT 42-1A | 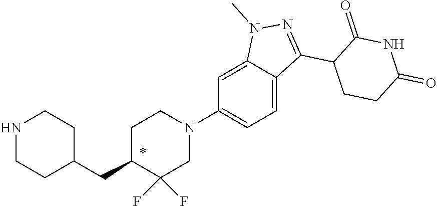 [SMILES: Cn1nc(C2CCC(=O)NC2=O)c2ccc(N3CC[C@H](CC4CCNCC4)C(F)(F)C3)cc21] | 460.3 | Steps 1-3 omitted. tert-butyl 3,3- difluoro-4-(hydroxymethyl)piperidine- 1-carboxylate in step 4; resulting enantiomers were separated via prep- SFC (Daicel Chrialpak AD 250 × 30 × 10 μm, CO₂/20% (0.5% MeO NH₃ in IPA). Peak 1 was selected and carried forward. |
| INT 42-1B | 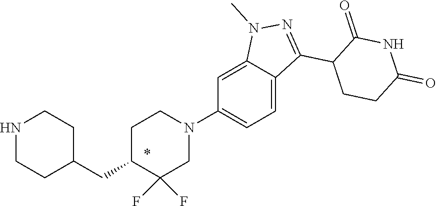 [SMILES: Cn1nc(C2CCC(=O)NC2=O)c2ccc(N3CC[C@@H](CC4CCNCC4)C(F)(F)C3)cc21] | 460.3 | Steps 1-3 omitted. tert-butyl 3,3- difluoro-4-(hydroxymethyl)piperidine- 1-carboxylate in step 4; resulting enantiomers were separated via prep- SFC (Daicel Chrialpak AD 250 × 30 × 10 μm, CO₂/20% (0.5% MeO NH₃ in IPA). Peak 2 was selected and carried forward. |
| INT 42-1C | 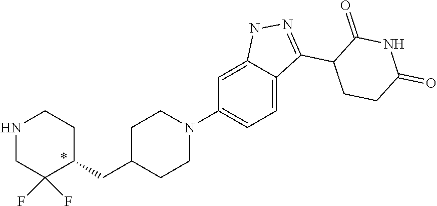 [SMILES: O=C1CCC(c2n[nH]c3cc(N4CCC(C[C@H]5CCNCC5(F)F)CC4)ccc23)C(=O)N1] | 460.3 | Steps 1-3 omitted. tert-butyl 3,3- difluoro-4-(hydroxymethyl)piperidine- 1-carboxylate in step 4; resulting enantiomers were separated via prep- SFC (Daicel Chrialpak AD 250 × 30 × 10 μm, CO₂/20% (0.5% MeO NH₃ in IPA). Peak 1 was carried forward. The product from step 5, tert-butyl-3,3- difluoro-4-(piperidin-4- ylmethyl)piperidine-1-carboxylate) was subsequently carried forward into Step 8. |
| INT 42-1D | 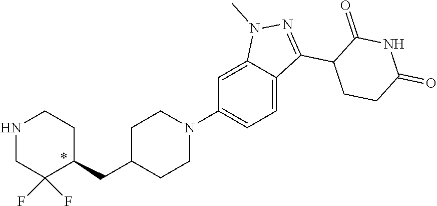 [SMILES: Cn1nc(C2CCC(=O)NC2=O)c2ccc(N3CCC(C[C@@H]4CCNCC4(F)F)CC3)cc21] | 460.3 | Steps 1-3 omitted. tert-butyl 3,3- difluoro-4-(hydroxymethyl)piperidine- 1-carboxylate in step 4; resulting enantiomers were separated via prep- SFC (Daicel Chrialpak AD 250 × 30 × 10 μm, CO₂/20% (0.5% MeO NH₃ in IPA). Peak 2 was carried forward. The product from step 5, tert-butyl -3,3- difluoro-4-(piperidin-4- ylmethyl)piperidine-1-carboxylate) was subsequently carried forward into Step 8. |

### Intermediates 42-1-42-2.

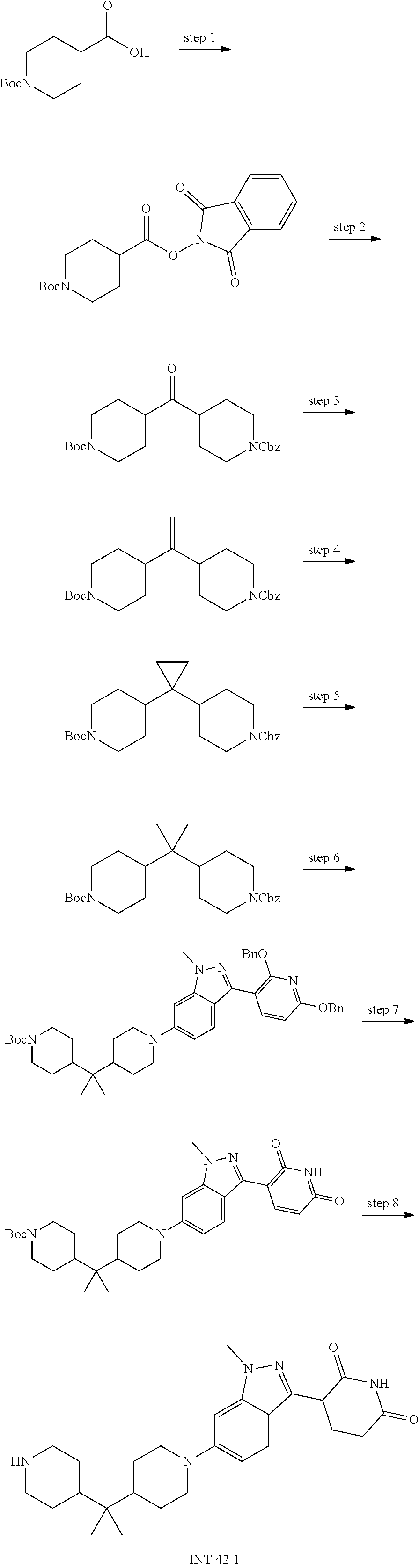

Step 1: 1-(tert-butyl) 4-(1,3-dioxoisoindolin-2-yl) piperidine-1,4-dicarboxylate. To a mixture of 2-hydroxyisoindoline-1,3-dione (7.5 g, 45 mmol) and 4-dimethylaminopyridine (0.53 g, 4.4 mmol) in dichloromethane (200 mL) was added 1-tert-butoxycarbonylpiperidine-4-carboxylic acid (10 g, 44 mmol), followed by dropwise addition of N,N′-diisopropylcarbodiimide (6.1 g, 48 mmol). The mixture was stirred at 25° C. for 1 h. The reaction mixture was filtered, and the filtrate was concentrated under reduced pressure to give a residue. The residue was purified by flash silica gel chromatography (0-20% EtOAc in PE) to afford the title compound. LCMS: 319.0.

Step 2: benzyl 4-(1-(tert-butoxycarbonyl)piperidine-4-carbonyl)piperidine-1-carboxylate. To a mixture of 1-tert-butyl 4-(1,3-dioxoisoindolin-2-yl)piperidine-1,4-dicarboxylate (5.0 g, 13 mmol), benzyl 4-(2-pyridylsulfanylcarbonyl)piperidine-1-carboxylate (4.8 g, 13 mmol), and [2,2′-bipyridine]-4,4′-dicarboxylate (0.073 g, 0.27 mmol) in tetrahydrofuran (20 mL) and N,N-dimethylacetamide (20 mL), was added (1,2-dimethoxyethane)nickel dibromide (0.082 g, 0.27 mmol), zinc chloride (0.36 g, 2.7 mmol), and zinc powder (1.8 g, 27 mmol). The mixture was stirred at 25° C. for 12 h under nitrogen. The reaction mixture was filtered and the filtrate was then diluted with water and extracted with ethyl acetate. The combined organic layers were washed with brine, dried over Na₂SO₄, filtered, and concentrated under reduced pressure. The crude residue was purified by silica gel chromatography (0-50% EtOAc in PE) to yield the title compound. LCMS: 331.2.

Step 3: benzyl 4-(1-(1-(tert-butoxycarbonyl)piperidin-4-yl)vinyl)piperidine-1-carboxylate. To a solution of methyl(triphenyl)phosphonium bromide (11.3 g, 31.6 mmol) in tetrahydrofuran (100 mL) was added n-butyllithium (2.5 M in hexanes, 12.6 mL, 31.6 mmol) dropwise at −20° C. under nitrogen. The mixture was stirred at 25° C. for 1.5 h. Benzyl 4-(1-(tert-butoxycarbonyl)piperidine-4-carbonyl)piperidine-1-carboxylate (6.8 g, 15.8 mmol) was then added, and the reaction was stirred at 25° C. for 12 h. The reaction mixture was quenched by the addition of saturated aqueous NH₄Cl solution at 0° C. and extracted with ethyl acetate. The combined organic layers were washed with brine, dried over Na₂SO₄, filtered, and concentrated under reduced pressure. The crude residue was purified by silica gel chromatography (0-20% EtOAc in PE) to yield the title compound. LCMS: 329.2.

Step 4: benzyl 4-(1-(1-(tert-butoxycarbonyl)piperidin-4-yl)cyclopropyl)piperidine-1-carboxylate. To a mixture of benzyl 4-(1-(1-(tert-butoxycarbonyl)piperidin-4-yl)vinyl)piperidine-1-carboxylate (3.9 g, 9.2 mmol) in tetrahydrofuran (50 mL) was added 2,6-bis(2,6-diisopropylphenyliminomethyl)pyridine (0.44 g, 0.92 mmol), cobalt(II) bromide (0.20 g, 0.92 mmol), and methylene bromide (3.2 g, 18 mmol). Zinc (1.2 g, 18 mmol) was then added in portions. The mixture was stirred at 25° C. for 12 h under nitrogen. The reaction mixture was filtered, and the filtrate was concentrated under reduced pressure to yield a residue. The filtrate cake was quenched with 1 M HCl, then the pH was adjusted to 8-9 using 1 M NaOH. The crude residue was purified by silica gel chromatography (0-20% EtOAc in PE) to yield the title compound. LCMS: 343.2

Step 5: tert-butyl 4-(2-(piperidin-4-yl)propan-2-yl)piperidine-1-carboxylate. To a solution of platinum(IV) oxide (410 mg, 1.8 mmol) in acetic acid (10 mL) was added benzyl 4-(1-(1-(tert-butoxycarbonyl)piperidin-4-yl)cyclopropyl)piperidine-1-carboxylate (410 mg, 0.93 mmol). The mixture was stirred at 25° C. for 24 h under hydrogen (50 psi). The suspension was filtered through a pad of Celite, which was washed with methanol. The combined filtrate was concentrated under reduced pressure to yield a residue which was purified by prep-HPLC (10% to 40% acetonitrile/water with 0.2% formic acid), and fractions containing the desired product were pooled and lyophilized to yield the title compound. LCMS: 311.2.

Step 6: tert-butyl 4-(2-(l-(3-(2,6-bis(benzyloxy)pyridin-3-yl)-1-methyl-1H-indazol-6-yl)piperidin-4-yl)propan-2-yl)piperidine-1-carboxylate. A vigorously stirred solution of tert-butyl 4-(2-(piperidin-4-yl)propan-2-yl)piperidine-1-carboxylate (0.80 mmol), 6-bromo-3-(2,6-dibenzyloxy-3-pyridyl)-1-methyl-indazole (0.60 mmol), tris(dibenzylideneacetone)dipalladium(0) (0.012 mmol), XPhos (0.018 mmol), and cesium carbonate (1.8 mmol) in 1,4-dioxane (6 mL) was heated under argon at 100° C. for 16 h. The reaction mixture was filtered through a short pad of Celite and washed with dichloromethane, then concentrated under reduced pressure. The crude residue was purified by silica gel chromatography (100% EtOAc) to yield the title compound. LCMS: 730.3

Step 7: tert-butyl 4-(2-(1-(3-(2,6-dioxopiperidin-3-yl)-1-methyl-1H-indazol-6-yl)piperidin-4-yl)propan-2-yl)piperidine-1-carboxylate. A mixture of palladium hydroxide on carbon (0.79 mmol) and tert-butyl 4-(2-(1-(3-(2,6-bis(benzyloxy)pyridin-3-yl)-1-methyl-1H-indazol-6-yl)piperidin-4-yl)propan-2-yl)piperidine-1-carboxylate (0.16 mmol) in THE (5 mL) was evacuated under vacuum and backfilled with hydrogen three times. The reaction mixture was stirred vigorously at room temperature for 12 h. Upon completion, the solution was filtered through a short pad of Celite, washed with dichloromethane, and concentrated under reduced pressure. The crude residue was purified by silica gel chromatography (0 to 50% EtOAc in hexanes) to yield the title compound. LCMS: 552.4.

Step 8: 3-(1-methyl-6-(4-(2-(piperidin-4-yl)propan-2-yl)piperidin-1-yl)-1H-indazol-3-yl)piperidine-2,6-dione (INT 42-1). Trifluoroacetic acid (0.5 mL) was added to a solution of tert-butyl 4-(2-(1-(3-(2,6-dioxopiperidin-3-yl)-1-methyl-1H-indazol-6-yl)piperidin-4-yl)propan-2-yl)piperidine-1-carboxylate (0.1 mmol) in dichloromethane (5 mL) at room temperature. The resulting mixture was stirred for 2 h. Upon completion, the reaction mixture was concentrated under reduced pressure and used directly in the next synthetic step without further purification. LCMS: 452.3.

The following intermediates were made in a similar fashion to INT 42-1 and are shown below in Table 2Y. To prepare the below intermediates, different reagents/starting materials were used than previously described in the steps toward INT 42-1 and are noted in the last column of Table 2Y—“Changes to Procedure: Different Reagents/Starting Materials”. A person of ordinary skill in the art will readily recognize which reagents/starting materials used for the synthesis of INT 42-1 were replaced with the different reagents/starting materials noted below.

TABLE 2Y
Intermediates

| | | | Changes to Procedure: Different |
|---|---|---|---|
| Intermediate | Structure | LCMS | Reagents/Starting Materials |
| INT 42-2 | 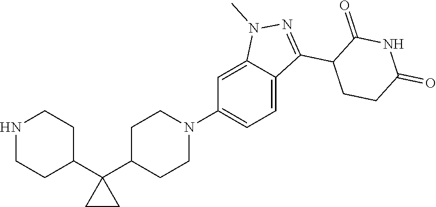 [SMILES: Cn1nc(C2CCC(=O)NC2=O)c2ccc(N3CCC(C4(C5CCNCC5)CC4)CC3)cc21] | 450.1 | 15 psi in step 5 to affortd tert-butyl 4- (1-(piperidin-4- yl)cyclopropyl)piperidine-1- carboxylate. |

### Intermediate 43-1.

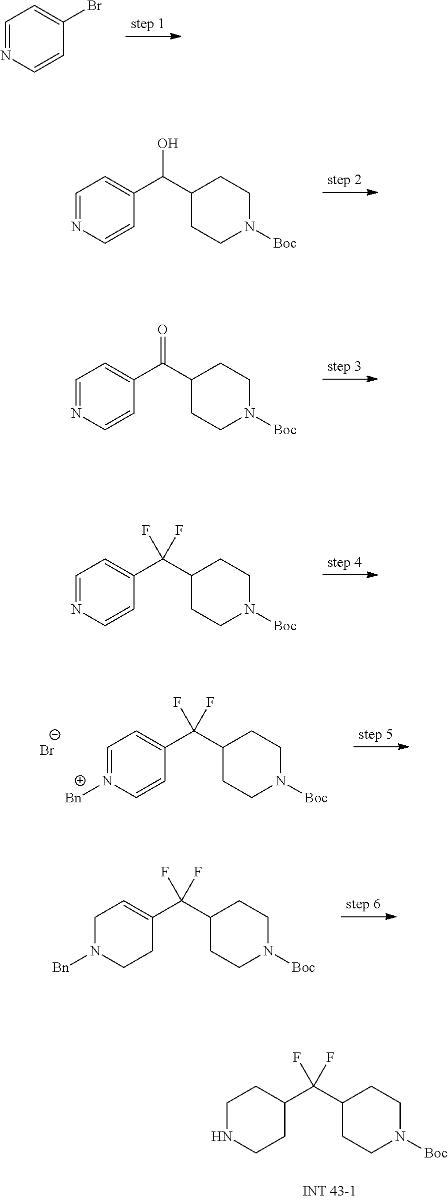

Step 1: tert-butyl 4-(hydroxy(pyridin-4-yl)methyl)piperidine-1-carboxylate. To a solution of 4-bromopyridine (3.2 mmol) in tetrahydrofuran (7.5 mL) was added isopropylmagnesium chloride (4.8 mmol) dropwise at 0° C. The mixture was then stirred at 0° C. for one hour under a nitrogen atmosphere. To the cooled mixture was then added dropwise a solution of tert-butyl 4-formylpiperidine-1-carboxylate (4.8 mmol) in tetrahydrofuran (1.5 mL). The mixture was then gradually warmed to room temperature and stirred for 5 hours under a nitrogen atmosphere. The mixture was then cooled to 0° C., quenched with saturated aqueous ammonium chloride solution (5 mL), and extracted with ethyl acetate (3×5 mL). The combined organic layers was then dried over sodium sulfate, filtered, and concentrated under reduced pressure. The resulting residue was then purified by flash column chromatography on silica gel eluting with ethyl acetate and petroleum ether to afford the title compound. LCMS: 293.1.

Step 2: tert-butyl 4-isonicotinoylpiperidine-1-carboxylate. To a solution of tert-butyl 4-(hydroxy(pyridin-4-yl)methyl)piperidine-1-carboxylate (17 mmol) in dichloromethane (40 mL) was added Dess-Martin Periodinane (26 mmol) at 0° C. The mixture was then warmed to room temperature and stirred for three hours. The mixture was then quenched with saturated aqueous sodium sulfite (15 mL) and sodium bicarbonate (15 mL) solutions and extracted with ethyl acetate (3×30 mL). The combined organic layers were dried over sodium sulfate, filtered, and concentrated under reduced pressure. The resulting residue was then purified by flash column chromatography on silica gel eluting with ethyl acetate and petroleum ether to afford the title compound. LCMS: 291.1.

Step 3: tert-butyl 4-(difluoro(pyridin-4-yl)methyl)piperidine-1-carboxylate. To tert-butyl 4-isonicotinoylpiperidine-1-carboxylate (0.2 mmol) was added diethylaminosulfur trifluoride (3.1 mmol), neat. The reaction mixture was stirred at room temperature for 24 hours under a nitrogen atmosphere. The mixture was then cooled to 0° C., quenched with saturated aqueous sodium bicarbonate solution (1 mL), and extracted with dichloromethane (3×1 mL). The combined organic layers were dried over sodium sulfate, filtered, and concentrated under reduced pressure. The resulting residue was then purified by flash column chromatography on silica gel eluting with ethyl acetate and petroleum ether to afford the title compound. LCMS: 313.1.

Step 4: 1-benzyl-4-((1-(tert-butoxycarbonyl)piperidin-4-yl)difluoromethyl)pyridin-1-ium bromide. To a solution of tert-butyl 4-(difluoro(pyridin-4-yl)methyl)piperidine-1-carboxylate (0.3 mmol) in acetonitrile (0.8 mL) was added (bromomethyl)benzene (0.4 mmol) and the mixture was stirred for one hour at 80° C. under a nitrogen atmosphere. The mixture was then concentrated under reduced pressure to afford the title compound without the need for further purification. LCMS: 403.2.

Step 5: tert-butyl 4-((1-benzyl-1,2,3,6-tetrahydropyridin-4-yl)difluoromethyl)piperidine-1-carboxylate. To a solution of 1-benzyl-4-((1-(tert-butoxycarbonyl)piperidin-4-yl)difluoromethyl)pyridin-1-ium bromide (0.3 mmol) in ethanol (1.2 mL) at 0° C. was added sodium borohydride (1.2 mmol). The mixture was then stirred for five hours at 50° C. under a nitrogen atmosphere. The reaction mixture was then quenched with saturated aqueous sodium bicarbonate solution (2 mL) and extracted with ethyl acetate (3×2 mL). The combined organic layers were dried over sodium sulfate, filtered, and concentrated under reduced pressure to afford the title compound without the need for further purification. LCMS: 407.2.

Step 6: tert-butyl 4-(difluoro(piperidin-4-yl)methyl)piperidine-1-carboxylate (INT 43-1): To a solution of tert-butyl 4-((1-benzyl-1,2,3,6-tetrahydropyridin-4-yl)difluoromethyl)piperidine-1-carboxylate (0.07 mmol) in ethanol (0.3 mL) was added rhodium on alumina (0.14 mmol, 5 wt. % Rh) under an argon atmosphere. The mixture was then evacuated and backfilled with hydrogen three times and stirred at room temperature under a hydrogen atmosphere for 24 hours. The mixture was then filtered, concentrated under reduced pressure, and purified by prep-HPLC (15% to 45%, acetonitrile/0.04% hydrochloric acid in water). Fractions containing the product were pooled and lyophilized to afford the title compound. LCMS: 319.1.

### Intermediate 43-2.

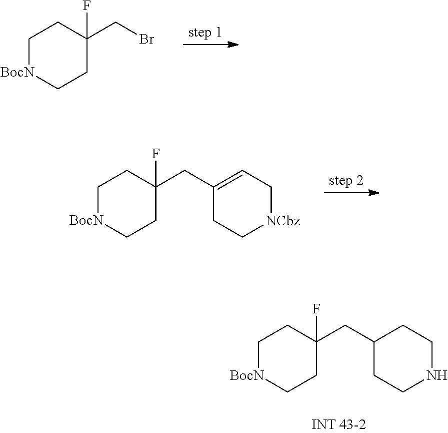 [SMILES: CC(C)(C)OC(=O)N1CCC(F)(CBr)CC1 ; CC(C)(C)OC(=O)N1CCC(F)(CC2=CCN(C(=O)OCc3ccccc3)CC2)CC1 ; CC(C)(C)OC(=O)N1CCC(F)(CC2CCNCC2)CC1]

Step 1: benzyl 4-((1-(tert-butoxycarbonyl)-4-fluoropiperidin-4-yl)methyl)-3,6-dihydropyridine-1(2H)-carboxylate. To a stirred solution of tert-butyl 4-(bromomethyl)-4-fluoropiperidine-1-carboxylate (1.0 mmol) and tert-butyl 4-(bromomethyl)-4-fluoro-piperidine-1-carboxylate (2.0 mmol) in DMF (7.5 mL) were added (2,2′-bipyridine)diiodonickel(II) (0.2 mmol), manganese (3.0 mmol), sodium iodide (0.5 mmol), chlorotrimethylsilane (0.2 mmol) at room temperature. The reaction mixture was stirred at 60° C. for 16 h. After completion, the reaction mixture was poured into cold water and extracted with ether. The combined organic layers were dried over sodium sulfate and concentrated under reduced pressure. The residue was purified by flash column chromatography on silica gel using 20% ethyl acetate in petroleum ether to afford the title compound. LCMS: 455.2.

Step 2: tert-butyl 4-fluoro-4-(piperidin-4-ylmethyl)piperidine-1-carboxylate (INT 43-2). A mixture of palladium hydroxide on carbon (30 mg) and benzyl 4-((1-(tert-butoxycarbonyl)-4-fluoropiperidin-4-yl)methyl)-3,6-dihydropyridine-1(2H)-carboxylate (0.49 mmol) in ethanol (5 mL) was evacuated under vacuum and backfilled with hydrogen three times. The reaction mixture was stirred at room temperature for 12 h. After completion, the mixture was filtered through a short pad of Celite, washed with dichloromethane, and concentrated under reduced pressure. The crude product was used directly in the next step without further purification. LCMS: 301.3.

### Intermediate 43-3.

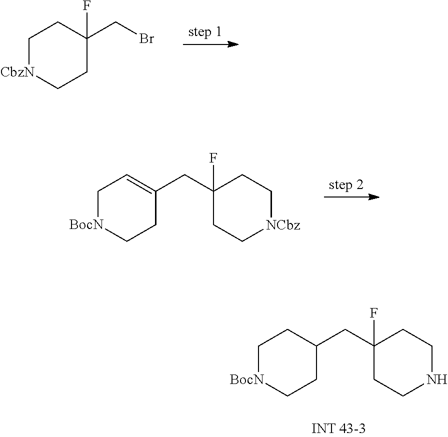 [SMILES: O=C(OCc1ccccc1)N1CCC(F)(CBr)CC1 ; CC(C)(C)OC(=O)N1CC=C(CC2(F)CCN(C(=O)OCc3ccccc3)CC2)CC1 ; CC(C)(C)OC(=O)N1CCC(CC2(F)CCNCC2)CC1]

Step 1: tert-butyl 4-((1-((benzyloxy)carbonyl)-4-fluoropiperidin-4-yl)methyl)-3,6-dihydropyridine-1(2H)-carboxylate. To a stirred solution of benzyl 4-(bromomethyl)-4-fluoropiperidine-1-carboxylate (15.1 mmol) and benzyl 4-(bromomethyl)-4-fluoro-piperidine-1-carboxylate (22.6 mmol) in DMF (100 mL), (2,2′-bipyridine)diiodonickel(II) (3 mmol), manganese (45.3 mmol), sodium iodide (7.55 mmol), and chlorotrimethylsilane (3.02 mmol) were added at room temperature. The reaction mixture was stirred at 60° C. for 16 h. After completion, the mixture was poured into cold water and extracted with ether. The combined organic layers were dried over sodium sulfate, filtered, and concentrated under reduced pressure. The residue was purified by flash column chromatography on silica gel (20% ethyl acetate in hexanes) to afford the title compound. LCMS: 455.3.

Step 2: tert-butyl 4-((4-fluoropiperidin-4-yl)methyl)piperidine-1-carboxylate (INT 43-3). A mixture of palladium hydroxide on carbon (500 mg) and tert-butyl 4-((1-((benzyloxy)carbonyl)-4-fluoropiperidin-4-yl)methyl)-3,6-dihydropyridine-1(2H)-carboxylate (2.3 mmol) in ethanol (7 mL) was evacuated under vacuum and backfilled with hydrogen three times. The reaction mixture was stirred vigorously at room temperature for 12 hours. Upon completion, the mixture was filtered through a short pad of celite and washed with dichloromethane. The filtrate was concentrated under reduced pressure. The resulting crude product was used directly in the next step without further purification. LCMS: 301.3.

### Intermediate 43-4.

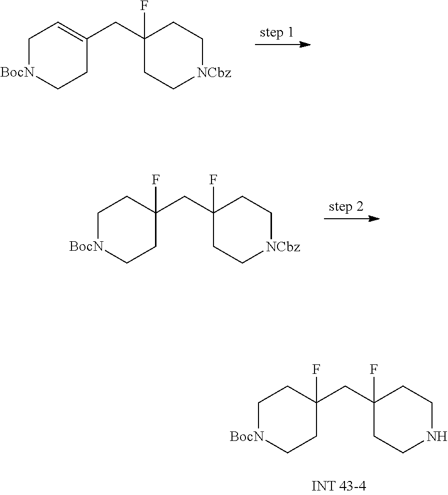 [SMILES: CC(C)(C)OC(=O)N1CC=C(CC2(F)CCN(C(=O)OCc3ccccc3)CC2)CC1 ; CC(C)(C)OC(=O)N1CCC(F)(CC2(F)CCN(C(=O)OCc3ccccc3)CC2)CC1 ; CC(C)(C)OC(=O)N1CCC(F)(CC2(F)CCNCC2)CC1]

Step 1: benzyl 4-((1-(tert-butoxycarbonyl)-4-fluoropiperidin-4-yl)methyl)-4-fluoropiperidine-1-carboxylate. To a stirred solution of tert-butyl 4-((1-((benzyloxy)carbonyl)-4-fluoropiperidin-4-yl)methyl)-3,6-dihydropyridine-1(2H)-carboxylate (10 mmol) in ethyl acetate (60 mL) and methanol (6 mL) were added nickel(II) nitrate hexahydrate (1.0 mmol), 1,1′-bis(diphenylphosphino)ferrocene (1.5 mmol), N-fluorobenzenesulfonimide (30 mmol), and polymethylhydrosiloxane (20 mmol) at room temperature. The reaction mixture was stirred at 50° C. for 16 h. After completion, the mixture was concentrated under reduced pressure. The residue was purified by flash column chromatography on silica gel using 20% ethyl acetate in petroleum ether to afford the title compound. LCMS: 453.4.

Step 2: tert-butyl 4-fluoro-4-((4-fluoropiperidin-4-yl)methyl)piperidine-1-carboxylate (INT 43-4). A suspension of palladium hydroxide on carbon (220 mg) and benzyl 4-((1-(tert-butoxycarbonyl)-4-fluoropiperidin-4-yl)methyl)-4-fluoropiperidine-1-carboxylate (2.3 mmol) in ethanol (5 mL) and tetrahydrofuran (2 mL) was evacuated under vacuum and backfilled with hydrogen three times. The reaction mixture was stirred vigorously at room temperature for 12 h. Upon completion, the mixture was filtered through a short pad of Celite, and the pad was washed with dichloromethane (3×10 mL). The combined filtrates were concentrated under reduced pressure to afford a crude residue, which was used directly in the next synthetic step without further purification. LCMS: 319.5.

### Intermediates 44-1-44-2.

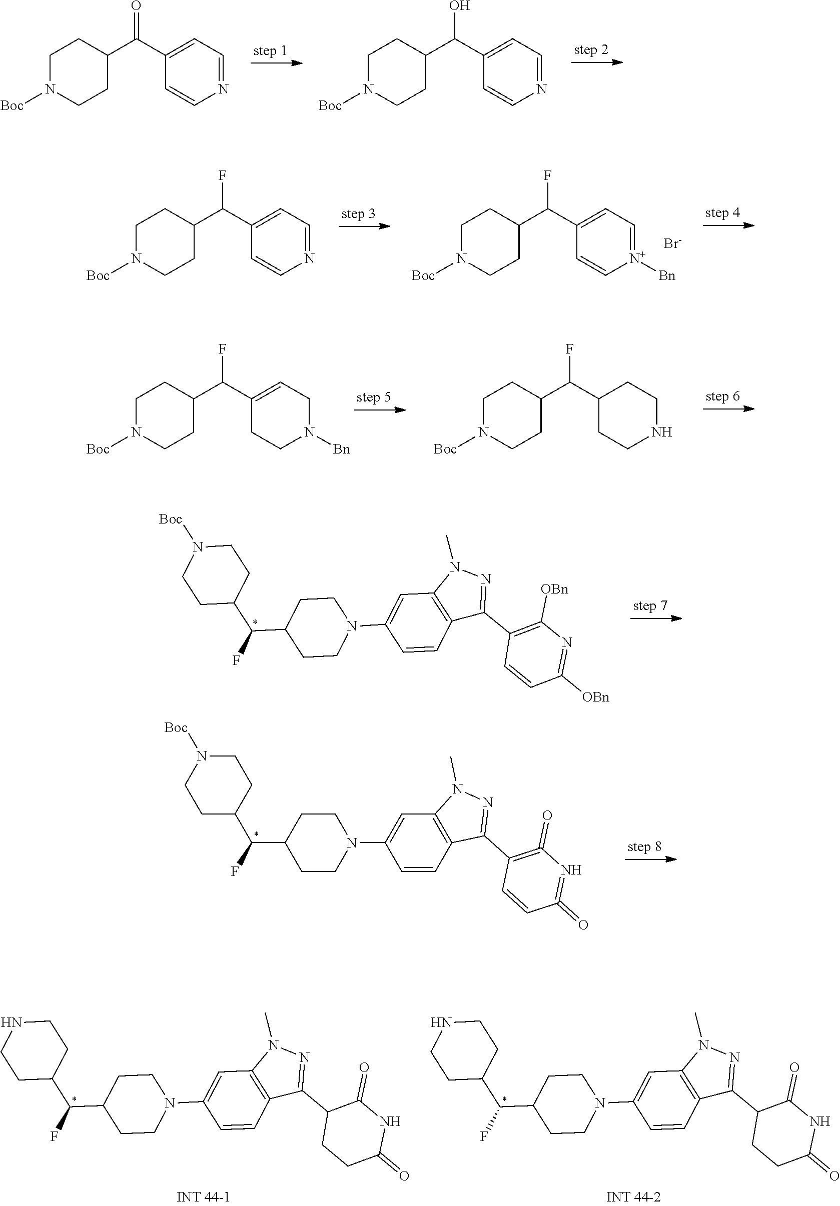

Step 1: tert-butyl 4-(hydroxy(pyridin-4-yl)methyl)piperidine-1-carboxylate. To a solution of tert-butyl 4-isonicotinoylpiperidine-1-carboxylate (5.0 g, 17 mmol) in methanol (80 mL) was added sodium borohydride (0.52 g, 14 mmol) at 0° C. and the reaction mixture was stirred for 3 h. Upon completion, the reaction mixture was concentrated under reduced pressure, quenched with water, and extracted with ethyl acetate. The organic layer was separated, dried over sodium sulfate, and concentrated under reduced pressure to afford the title compound. LCMS: 343.3.

Step 2: tert-butyl 4-(fluoro(pyridin-4-yl)methyl)piperidine-1-carboxylate. To a stirred solution of tert-butyl 4-(hydroxy(pyridin-4-yl)methyl)piperidine-1-carboxylate (4.9 g, 11 mmol,) in dichloromethane (80 mL) was added diethylaminosulfur trifluoride (3.0 mL, 23 mmol) at 0° C. The mixture was stirred at room temperature for 2.0 h. After completion, the reaction was quenched with saturated sodium bicarbonate solution and extracted with dichloromethane. The combined organic layers were washed with brine, dried over Na₂SO₄, filtered, and concentrated under reduced pressure. The crude residue was purified by silica gel chromatography (30% EtOAc in PE) to yield the title compound. LCMS: 336.2.

Step 3: 1-benzyl-4-((1-(tert-butoxycarbonyl)piperidin-4-yl)fluoromethyl)pyridin-1-ium. To a solution of tert-butyl 4-(fluoro(pyridin-4-yl)methyl)piperidine-1-carboxylate (2.4 g, 8.2 mmol) in acetonitrile (25 mL) was added benzyl bromide (1.7 g, 9.8 mmol) at room temperature and the mixture was stirred for 12 h. Upon completion, the reaction mixture was concentrated under reduced pressure to afford the title compound. LCMS: 385.4.

Step 4: tert-butyl 4-((1-benzyl-1,2,3,6-tetrahydropyridin-4-yl)fluoromethyl)piperidine-1-carboxylate. To a stirred solution of 1-benzyl-4-((1-(tert-butoxycarbonyl)piperidin-4-yl)fluoromethyl)pyridin-1-ium (2.6 g, 6.7 mmol) in methanol (26 mL) was added sodium borohydride (0.26 g, 6.7 mmol) at 0° C. The mixture was stirred at room temperature for 2 h. Upon completion, the reaction mixture was concentrated under reduced pressure to give a residue which was purified by flash silica gel chromatography (10% MeOH in DCM) to afford the title compound. LCMS: 389.4.

Step 5: tert-butyl 4-(fluoro(piperidin-4-yl)methyl)piperidine-1-carboxylate. To a stirred solution of tert-butyl 4-((1-benzyl-1,2,3,6-tetrahydropyridin-4-yl)fluoromethyl)piperidine-1-carboxylate (0.50 g, 1.3 mmol) in methanol (10 mL) was added 5% rhodium on alumina (0.50 g, wt/wt) at room temperature. The reaction mixture was stirred under a hydrogen atmosphere for 12 h. After completion, the mixture was filtered through a Celite bed, and the filtrate was concentrated under reduced pressure to afford the title compound. LCMS: 301.3.

Step 6. tert-butyl (S)-4-((1-(3-(2,6-bis(benzyloxy)pyridin-3-yl)-1-methyl-1H-indazol-6-yl)piperidin-4-yl)fluoromethyl)piperidine-1-carboxylate and tert-butyl (R)-4-((1-(3-(2,6-bis(benzyloxy)pyridin-3-yl)-1-methyl-1H-indazol-6-yl)piperidin-4-yl)fluoromethyl)piperidine-1-carboxylate. To a solution of tert-butyl 4-(fluoro(piperidin-4-yl)methyl)piperidine-1-carboxylate (0.5 g, 1.7 mmol) and 3-(2,6-bis(benzyloxy)pyridin-3-yl)-6-bromo-1-methyl-1H-indazole (0.6 g, 1.2 mmol) in 1,4-dioxane (10 mL) were added sodium tert-butoxide (400 mg, 4.2 mmol, 2.5 eq), palladium(II) acetate (76 mg, 0.08 mmol), RuPhos (78 mg, 0.17 mmol) and an additional 10 mL of 1,4-dioxane. Purging with nitrogen was continued for 5 minutes, and the mixture was stirred at 100° C. for 12 h. After completion, the reaction mixture was filtered through a Celite. The filtrate was washed with water and extracted with ethyl acetate. The organic layer was separated, dried over sodium sulfate, and concentrated under reduced pressure to afford the crude residue. The residue was then purified by flash column chromatography on silica gel eluting with ethyl acetate and hexanes. The residue was subsequently further separated by chiral SFC (Chiralpak IG packed 150×25×5 pm) n-hexane/ethanol (1:4) into two isomers, arbitrarily labeled as (R*)-4-((1-(3-(2,6-bis(benzyloxy)pyridin-3-yl)-1-methyl-1H-indazol-6-yl)piperidin-4-yl)fluoromethyl)piperidine-1-carboxylate and (S*)-4-((1-(3-(2,6-bis(benzyloxy)pyridin-3-yl)-1-methyl-1H-indazol-6-yl)piperidin-4-yl)fluoromethyl)piperidine-1-carboxylate. LCMS: 720.9.

Step 7. tert-butyl 4-((1R*)-(1-(3-(2,6-dioxopiperidin-3-yl)-1-methyl-1H-indazol-6-yl)piperidin-4-yl)fluoromethyl)piperidine-1-carboxylate. To a solution of tert-butyl (R*)-4-((1-(3-(2,6-bis(benzyloxy)pyridin-3-yl)-1-methyl-1H-indazol-6-yl)piperidin-4-yl)fluoromethyl)piperidine-1-carboxylate (80 mg, 0.11 mmol) in THE (5 mL) was added palladium hydroxide on carbon (80 mg, wt/wt, 20% Pd, ca. 60% moisture) and palladium on carbon (80 mg, wt/wt, 10% Pd, ca. 55% water). The mixture was stirred at room temperature under a hydrogen balloon for 5 h. After completion, the reaction mixture was filtered through a Celite bed and washed with tetrahydrofuran. The filtrate was concentrated under reduced pressure to afford the crude residue which was purified by flash column chromatography on silica gel eluting with ethyl acetate and hexanes to yield the title compound. LCMS: 542.6.

Step 8. 3-(6-(4-((S*)-fluoro(piperidin-4-yl)methyl)piperidin-1-yl)-1-methyl-1H-indazol-3-yl)piperidine-2,6-dione (INT 44-1). To a solution of tert-butyl 4-((1R*)-(1-(3-(2,6-dioxopiperidin-3-yl)-1-methyl-1H-indazol-6-yl)piperidin-4-yl)fluoromethyl)piperidine-1-carboxylate (28 mg) in dichloromethane (3.0 mL) was added trifluoroacetic acid (0.6 mL) dropwise. The reaction mixture was stirred at room temperature for 3 hours. After completion, the reaction mixture was concentrated to afford the title compound. LCMS: 442.3.

3-(6-(4-((R*)-fluoro(piperidin-4-yl)methyl)piperidin-1-yl)-1-methyl-1H-indazol-3-yl)piperidine-2,6-dione (INT 44-2) was prepared using (S*)-4-((1-(3-(2,6-bis(benzyloxy)pyridin-3-yl)-1-methyl-1H-indazol-6-yl)piperidin-4-yl)fluoromethyl)piperidine-1-carboxylate, isolated in step 6. LCMS: 442.3.

### Intermediates 45-1-45-2.

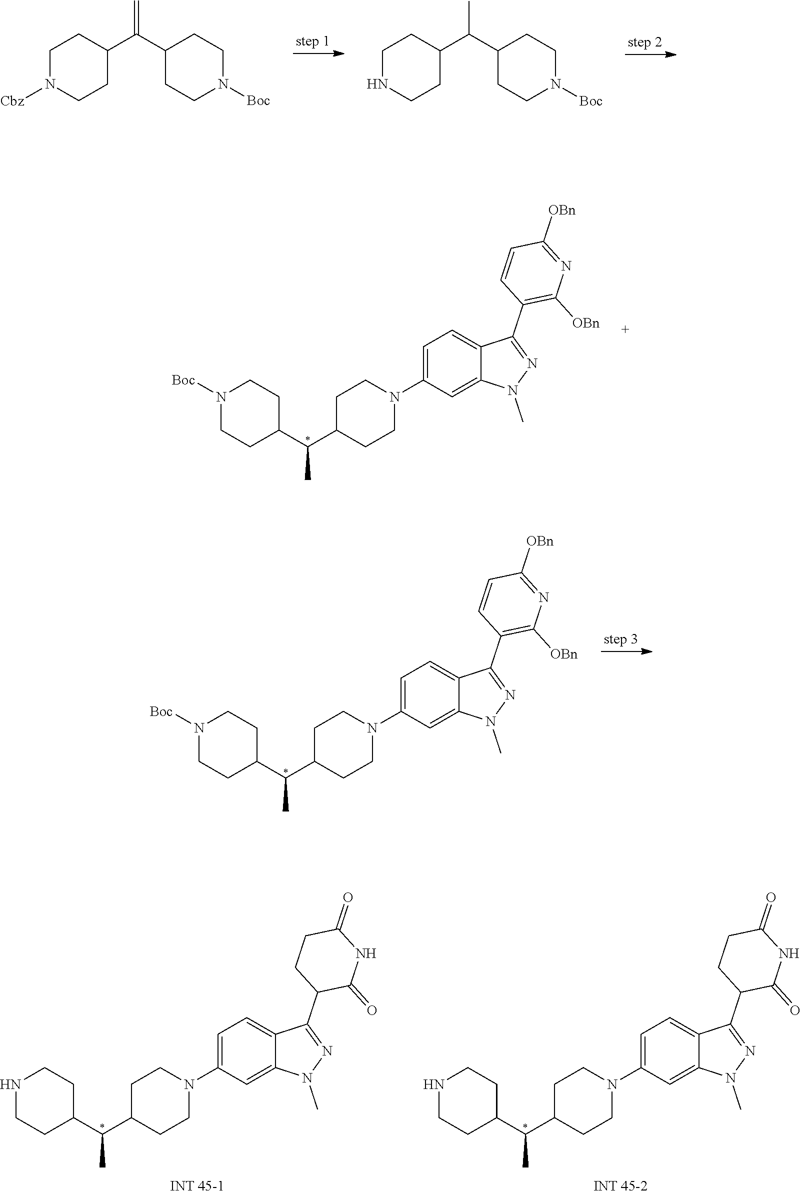 [SMILES: C=C(C1CCN(C(=O)OCc2ccccc2)CC1)C1CCN(C(=O)OC(C)(C)C)CC1 ; CC(C1CCNCC1)C1CCN(C(=O)OC(C)(C)C)CC1 ; C[C@@H](C1CCN(C(=O)OC(C)(C)C)CC1)C1CCN(c2ccc3c(-c4ccc(OCc5ccccc5)nc4OCc4ccccc4)nn(C)c3c2)CC1 ; C[C@@H](C1CCN(C(=O)OC(C)(C)C)CC1)C1CCN(c2ccc3c(-c4ccc(OCc5ccccc5)nc4OCc4ccccc4)nn(C)c3c2)CC1 ; C[C@@H](C1CCNCC1)C1CCN(c2ccc3c(C4CCC(=O)NC4=O)nn(C)c3c2)CC1 ; C[C@@H](C1CCNCC1)C1CCN(c2ccc3c(C4CCC(=O)NC4=O)nn(C)c3c2)CC1]

Step 1: tert-butyl 4-(1-(piperidin-4-yl)ethyl)piperidine-1-carboxylate. To a solution of benzyl 4-(1-(1-(tert-butoxycarbonyl)piperidin-4-yl)vinyl)piperidine-1-carboxylate (0.20 mmol) in methanol (1.0 mL) was added palladium on carbon (0.30 mmol, 10 wt % Pd). The mixture was evacuated and backfilled with hydrogen three times, then stirred at room temperature under a hydrogen atmosphere for 16 h. After completion, the mixture was filtered through celite and concentrated under reduced pressure to afford the title compound. LCMS: 297.0.

Step 2: tert-butyl (R)-4-(1-(1-(3-(2,6-bis(benzyloxy)pyridin-3-yl)-1-methyl-1H-indazol-6-yl)piperidin-4-yl)ethyl)piperidine-1-carboxylate and tert-butyl (S)-4-(1-(1-(3-(2,6-bis(benzyloxy)pyridin-3-yl)-1-methyl-1H-indazol-6-yl)piperidin-4-yl)ethyl)piperidine-1-carboxylate. To a solution of tert-butyl 4-(1-(piperidin-4-yl)ethyl)piperidine-1-carboxylate (0.03 mmol) in 1,4-dioxane (0.3 mL) was added 3-(2,6-bis(benzyloxy)pyridin-3-yl)-6-bromo-1-methyl-1H-indazole (0.03 mmol), cesium carbonate (0.10 mmol), X-Phos (0.003 mmol), and tris(dibenzylideneacetone)dipalladium(0) (0.002 mmol). The mixture was then degassed with nitrogen for five minutes and stirred at 100° C., under a nitrogen atmosphere, for 16 hours. The reaction mixture was then filtered, concentrated under reduced pressure, and purified by flash column chromatography on silica gel eluting with ethyl acetate and hexanes. The residue was further separated by chiral SFC (Chiralpak AD-H 250×4.6×5 pm) n-hexane/ethanol (1:4) with 0.2% diethylamine into two enantiomers, arbitrarily labeled as (R*)-4-(1-(1-(3-(2,6-bis(benzyloxy)pyridin-3-yl)-1-methyl-1H-indazol-6-yl)piperidin-4-yl)ethyl)piperidine-1-carboxylate (Isomer 1) and tert-butyl (S*)-4-(1-(1-(3-(2,6-bis(benzyloxy)pyridin-3-yl)-1-methyl-1H-indazol-6-yl)piperidin-4-yl)ethyl)piperidine-1-carboxylate (Isomer 2). LCMS: 720.9.

Step 3: 3-(1-methyl-6-(4-((S*)-1-(piperidin-4-yl)ethyl)piperidin-1-yl)-1H-indazol-3-yl)piperidine-2,6-dione (INT 45-1): To a solution of tert-butyl (R*)-4-(1-(1-(3-(2,6-bis(benzyloxy)pyridin-3-yl)-1-methyl-1H-indazol-6-yl)piperidin-4-yl)ethyl)piperidine-1-carboxylate (0.70 mmol) in tetrahydrofuran (5.0 mL) was added palladium hydroxide on carbon (3.0 mmol, 20 wt % Pd). The mixture was evacuated and backfilled with hydrogen three times, then stirred at room temperature under a hydrogen atmosphere for 6 hours. The mixture was filtered through a Celite pad and concentrated under reduced pressure. The resulting residue was dissolved in dichloromethane (10.0 mL), and trifluoroacetic acid (1.2 mL) was added. The mixture was stirred at room temperature for one hour, then concentrated under reduced pressure to afford the title compound. LCMS: 438.2.

3-(1-methyl-6-(4-((R*)-1-(piperidin-4-yl)ethyl)piperidin-1-yl)-1H-indazol-3-yl)piperidine-2,6-dione (INT 45-2) was prepared using (S*)-4-(1-(1-(3-(2,6-bis(benzyloxy)pyridin-3-yl)-1-methyl-1H-indazol-6-yl)piperidin-4-yl)ethyl)piperidine-1-carboxylate, isolated in step 2. LCMS: 438.2.

### Intermediate 46-1.

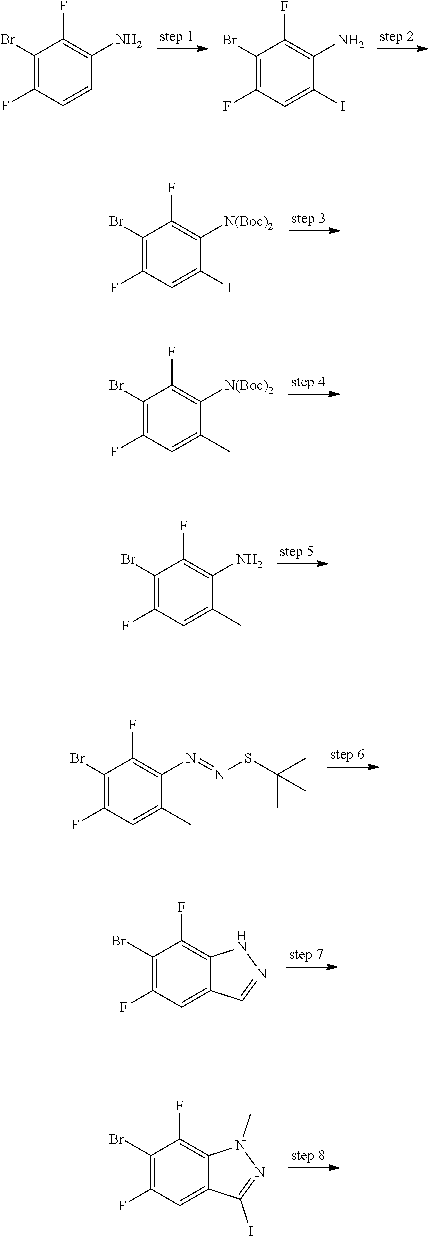

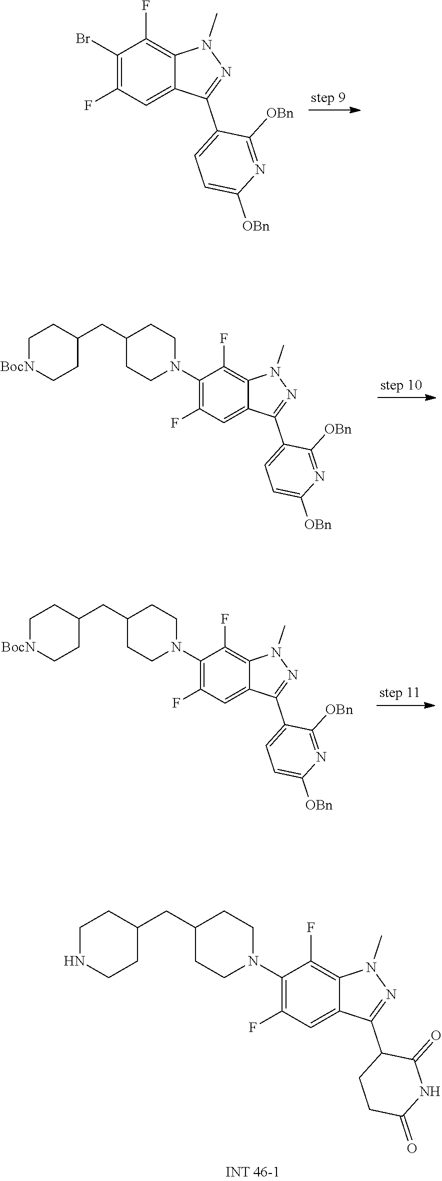 [SMILES: Cn1nc(-c2ccc(OCc3ccccc3)nc2OCc2ccccc2)c2cc(F)c(Br)c(F)c21 ; Cn1nc(-c2ccc(OCc3ccccc3)nc2OCc2ccccc2)c2cc(F)c(N3CCC(CC4CCN(C(=O)OC(C)(C)C)CC4)CC3)c(F)c21 ; Cn1nc(-c2ccc(OCc3ccccc3)nc2OCc2ccccc2)c2cc(F)c(N3CCC(CC4CCN(C(=O)OC(C)(C)C)CC4)CC3)c(F)c21 ; Cn1nc(C2CCC(=O)NC2=O)c2cc(F)c(N3CCC(CC4CCNCC4)CC3)c(F)c21]

Step 1: 3-bromo-2,4-difluoro-6-iodoaniline. A solution of 3-bromo-2,4-difluoroaniline (1.2 mol) in ethanol (2.5 L) was treated with iodine (4.9 mol) and silver sulfate (1.7 mol) at room temperature. The mixture was stirred for 1 h. Upon completion, the reaction mixture was concentrated and purified by silica gel chromatography to afford the title compound. LCMS: 334.0

Step 2: tert-butyl (3-bromo-2,4-difluoro-6-iodophenyl)(tert-butoxycarbonyl)carbamate. To a solution of 3-bromo-2,4-difluoro-6-iodoaniline (0.74 mol) in 1,4-dioxane (2.5 L) was added 4-dimethylaminopyridine (74 mmol), triethylamine (3 mol), and di-tert-butyl dicarbonate (1.9 mol). The mixture was stirred at 70° C. for 2 h. The mixture was concentrated in vacuo to give a residue, which was triturated with petroleum ether/ethyl acetate to afford the title compound.

Step 3: tert-butyl (3-bromo-2,4-difluoro-6-methylphenyl)(tert-butoxycarbonyl)carbamate. To a solution of tert-butyl (3-bromo-2,4-difluoro-6-iodophenyl)(tert-butoxycarbonyl)carbamate (290 mmol) in 1,4-dioxane (1.2 L) and water (300 mL) was added methylboronic acid (1.7 mol), potassium carbonate (0.58 mol) and bis(triphenylphosphine)palladium(II) dichloride (29 mmol,). The reaction mixture was stirred under N₂ atmosphere at 100° C. for 8 h. the reaction mixture was diluted with water, extracted with ethyl acetate and washed with brine. The organic layer was dried over Na₂SO₄ and concentrated to give a crude residue, which was purified by silica gel column chromatography to afford the title compound. LCMS: 444.1.

Step 4: 3-bromo-2,4-difluoro-6-methylaniline hydrogen chloride. To a solution of tert-butyl (3-bromo-2,4-difluoro-6-methylphenyl)(tert-butoxycarbonyl)carbamate (0.3 mmol) in dichloromethane (60 mL) was added hydrogen chloride in 1,4-dioxane (0.6 mol). The mixture was stirred at room temperature for 15 h. The mixture was concentrated to afford the title compound. LCMS: 222.2.

Step 5: (E)-1-(3-bromo-2,4-difluoro-6-methylphenyl)-2-(tert-butylthio)diazene. To a solution of 3-bromo-2,4-difluoro-6-methylaniline hydrochloride (0.39 mol) in hydrochloric acid (6.6 mol) was added sodium nitrite (0.43 mol) in water (100 mL) at 0° C. and the mixture was stirred at 0° C. for 0.5 h. 2-Methyl-2-propanethiol (0.45 mol) in ethanol (200 mL). was added and the mixture was stirred at 0° C. for 2 h. Upon completion, the mixture was diluted with water, extracted with dichloromethane, washed with brine, dried over sodium sulfate, filtered, and concentrated. The crude residue was purified by silica gel column chromatography to afford the title compound.

Step 6: 6-bromo-5,7-difluoro-1H-indazole. To a solution of (E)-1-(3-bromo-2,4-difluoro-6-methylphenyl)-2-(tert-butylthio)diazene (0.26 mol) in tetrahydrofuran (0.83 L) was added potassium tert-butoxide in tetrahydrofuran (1 M, 0.5 mmol). The mixture was stirred at room temperature for 1 h. Water (0.2 L) and formic acid (40 mL) were added, and the mixture was extracted with ethyl acetate. The combined organic phases were washed with brine, dried over sodium sulfate, filtered, and concentrated to afford a crude residue, which was triturated with petroleum ether/ethyl acetate to yield the title compound. LCMS: 232.9.

Step 7: 6-bromo-5,7-difluoro-3-iodo-1-methyl-1H-indazole. To a solution of 6-bromo-5,7-difluoro-1H-indazole (0.18 mol, 1) in dimethylformamide (0.42 L) was added potassium hydroxide (0.45 mol) and iodine (0.31 mol). The reaction mixture was stirred at room temperature for 1 h. Iodomethane (0.27 mol) was added, and the mixture was stirred at room temperature for an additional 2 h. The mixture was diluted with water was added, and extracted with ethyl acetate. The combined organic fractions were washed sodium sulfite solution and brine (400 mL), dried over sodium sulfate, filtered, and concentrated to yield a crude product. The residue was purified by silica gel column chromatography to afford the title compound. LCMS: 372.8

Step 8: 3-(2,6-bis(benzyloxy)pyridin-3-yl)-6-bromo-5,7-difluoro-1-methyl-1H-indazole. To a solution of 6-bromo-5,7-difluoro-3-iodo-1-methyl-1H-indazole (27 mmol) in 1,4-dioxane (150 mL) and water (50 mL) was added 3-(2,6-bis(benzyloxy)pyridin-3-yl)-6-bromo-1-methyl-1H-indazole (28 mmol), cesium carbonate (67 mmol), and bis(diphenylphosphino)ferrocene)palladium(II) dichloride (2.7 mmol). The mixture was stirred at 90° C. for 3 h. The mixture was diluted with water (300 mL) and extracted with ethyl. The combined organic fractions were washed with brine, dried over sodium sulfate, filtered, and concentrated to yield a crude product. The residue was purified by silica gel column chromatography to afford the title compound. LCMS: 535.9.

Step 9: tert-butyl 4-[[1-[3-(2,6-dibenzyloxy-3-pyridyl)-5,7-difluoro-1-methyl-indazol-6-yl]-4-piperidyl]methyl]piperidine-1-carboxylate. To a solution of 6-bromo-3-(2,6-dibenzyloxy-3-pyridyl)-5,7-difluoro-1-methyl-indazole (2.6 mmol) and tert-butyl 4-(4-piperidylmethyl)piperidine-1-carboxylate (7.8 mmol) in 2-methylbutan-2-ol (3.0 mL) was added GPhos Pd G6 TES (0.26 mmol) and sodium tert-butoxide (2.0 M in 2.6 mL). The reaction mixture was stirred at 90° C. for 1 h under nitrogen atmosphere. Upon completion, the mixture was filtered, and concentrated under reduced pressure. The residue was purified by flash silica gel chromatography to afford the title compound. LCMS: 738.3.

Step 10: tert-butyl 4-[[1-[3-(2,6-dioxo-3-piperidyl)-5,7-difluoro-1-methyl-indazol-6-yl]-4-piperidyl]methyl]piperidine-1-carboxylate. A solution of tert-butyl 4-[[1-[3-(2,6-dibenzyloxy-3-pyridyl)-5,7-difluoro-1-methyl-indazol-6-yl]-4-piperidyl]methyl]piperidine-1-carboxylate (1.4 mmol) in ethyl acetate (20 mL) and dimethylformamide (2 mL) was added to Pd/C (10%, 0.94 mmol). The suspension was degassed under vacuum and purged with hydrogen. The mixture was then stirred under hydrogen (15 psi) at 30° C. for 1 h. The mixture was filtered, and concentrated under reduced pressure. Water was added, and the resulting filter cake was collected to yield the title compound, which was used without purification. LCMS: 560.2.

Step 11: 3-[5,7-difluoro-1-methyl-6-[4-(4-piperidylmethyl)-1-piperidyl]indazol-3-yl]piperidine-2,6-dione (INT 46-1). To a solution of tert-butyl 4-[[1-[3-(2,6-dioxo-3-piperidyl)-5,7-difluoro-1-methyl-indazol-6-yl]-4-piperidyl]methyl]piperidine-1-carboxylate (1.2 mmol) in ethyl acetate (5 mL) was added hydrochloric acid (5 mL). The mixture was stirred at 20° C. for 1 h. Upon completion, the reaction was concentrated and purified by RP-HPLC (0 to 40%, 0.04% HCl in acetonitrile/0.04% HCl in water). Fractions containing the product were pooled and lyophilized to yield the title compound. LCMS: 460.2.

### Intermediate 47-1.

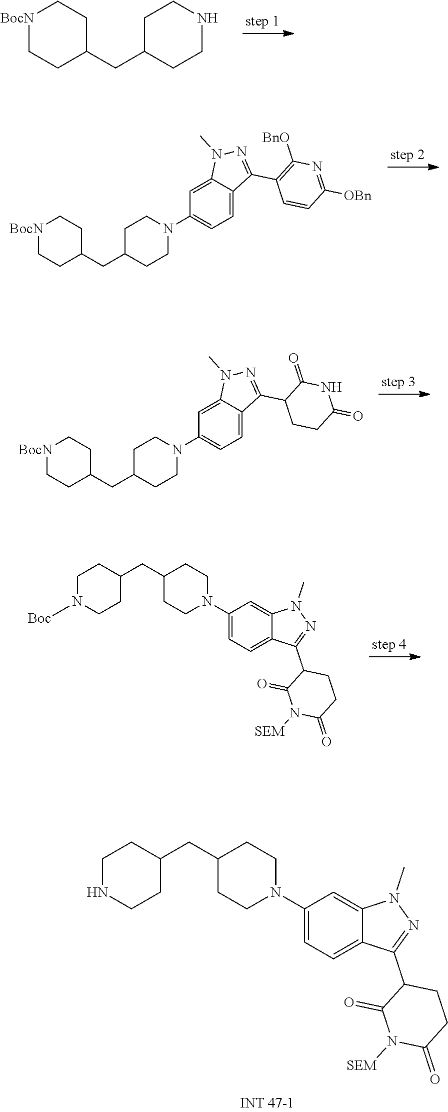 [SMILES: CC(C)(C)OC(=O)N1CCC(CC2CCNCC2)CC1 ; Cn1nc(-c2ccc(OCc3ccccc3)nc2OCc2ccccc2)c2ccc(N3CCC(CC4CCN(C(=O)OC(C)(C)C)CC4)CC3)cc21 ; Cn1nc(C2CCC(=O)NC2=O)c2ccc(N3CCC(CC4CCN(C(=O)OC(C)(C)C)CC4)CC3)cc21 ; Cn1nc(C2CCC(=O)N(COCC[Si](C)(C)C)C2=O)c2ccc(N3CCC(CC4CCN(C(=O)OC(C)(C)C)CC4)CC3)cc21 ; Cn1nc(C2CCC(=O)N(COCC[Si](C)(C)C)C2=O)c2ccc(N3CCC(CC4CCNCC4)CC3)cc21]

Step 1: tert-butyl 4-((1-(3-(2,6-bis(benzyloxy)pyridin-3-yl)-1-methyl-1H-indazol-6-yl)piperidin-4-yl)methyl)piperazine-1-carboxylate. A vigorously stirred solution of tert-butyl 4-(piperidin-4-ylmethyl)piperidine-1-carboxylate (0.8 mmol), 6-bromo-3-(2,6-dibenzyloxy-3-pyridyl)-1-methyl-indazole (0.6 mmol), tris(dibenzylideneacetone)dipalladium(0) (0.012 mmol), 2-dicyclohexylphosphino-2′,4′,6′-triisopropylbiphenyl (0.018 mmol), and cesium carbonate (1.8 mmol) in 1,4-dioxane (6 mL) was heated under argon at 100° C. for 16 hours. The reaction mixture was filtered through a short pad of Celite and washed with dichloromethane (3×10 mL), then concentrated under reduced pressure. The residue was purified by flash column chromatography on silica gel (100% ethyl acetate) to afford the title compound. LCMS: 702.2.

Step 2: tert-butyl 4-((1-(3-(2,6-dioxopiperidin-3-yl)-1-methyl-1H-indazol-6-yl)piperidin-4-yl)methyl)piperidine-1-carboxylate. A mixture of palladium hydroxide on carbon (2.8 mmol, 20 wt. % Pd) and tert-butyl 4-((1-(3-(2,6-bis(benzyloxy)pyridin-3-yl)-1-methyl-1H-indazol-6-yl)piperidin-4-yl)methyl)piperazine-1-carboxylate (0.55 mmol) in tetrahydrofuran (5 mL) was evacuated and backfilled with hydrogen three times. The reaction mixture was vigorously stirred at room temperature for 12 hours. Upon completion, the solution was filtered through a short pad of Celite, washed with dichloromethane (3×10 mL), then concentrated under reduced pressure. The residue was purified by flash column chromatography on silica gel (0% to 50% ethyl acetate in hexanes) to afford the title compound. LCMS: 524.1.

Step 3: tert-butyl 4-((1-(3-(2,6-dioxo-1-((2-(trimethylsilyl)ethoxy)methyl)piperidin-3-yl)-1-methyl-1H-indazol-6-yl)piperidin-4-yl)methyl)piperidine-1-carboxylate. tert-butyl 4-[[11-[3-(2,6-dioxo-3-piperidyl)-1-methyl-indazol-6-yl]-4-piperidyl]methyl]piperidine-1-carboxylate (11.0 g, 0.0210 mol) in N,N-dimethylformamide (120 mL) was cooled to 0° C., and then 1,8-diazabicyclo[5.4.0]undec-7-ene (32.0 g, 0.210 mol) was added. Then (2-(chloromethoxy)ethyl)trimethylsilane (10.5 g, 0.0630 mol) was added. The reaction mixture was allowed to warm to room temperature and stirred for 2 h. The reaction mixture was partitioned between EtOAc (150 mL) and water (150 mL), and the aqueous phase was extracted with EtOAc (150 mL×3). The combined organic layer was dried over Na₂SO₄, filtered, and concentrated under reduced pressure. The residue was purified by flash silica gel chromatography eluting with 0-20% EtOAc/hexanes to yield the title compound. LCMS: 654.5

Step 2: 3-(1-methyl-6-(4-(piperidin-4-ylmethyl)piperidin-1-yl)-1H-indazol-3-yl)-1-((2-(trimethylsilyl)ethoxy)methyl)piperidine-2,6-dione (INT 47-1). To a mixture of tert-butyl 4-[[1-[3-[2,6-dioxo-1-(2-trimethylsilylethoxymethyl)-3-piperidyl]-1-methyl-indazol-6-yl]-4-piperidyl]methyl]piperidine-1-carboxylate (2.0 g, 3.1 mmol) in DCM (25 mL) was added 2,6-lutidine (3.3 g, 31 mmol) at 0° C. The mixture was stirred at 0° C. for 10 min. A solution of trimethylsilyl trifluoromethanesulfonate (1.4 g, 6.1 mmol) in DCM (2.0 mL) was then added dropwise. The reaction mixture was allowed to warm to room temperature and stirred for 2 h. It was then concentrated under reduced pressure to yield a residue, which was purified by reverse-phase preparative HPLC (5-40% acetonitrile/water containing 0.04% HCl) to afford the title compound. LCMS: 554.4.

### Intermediate 48-1.

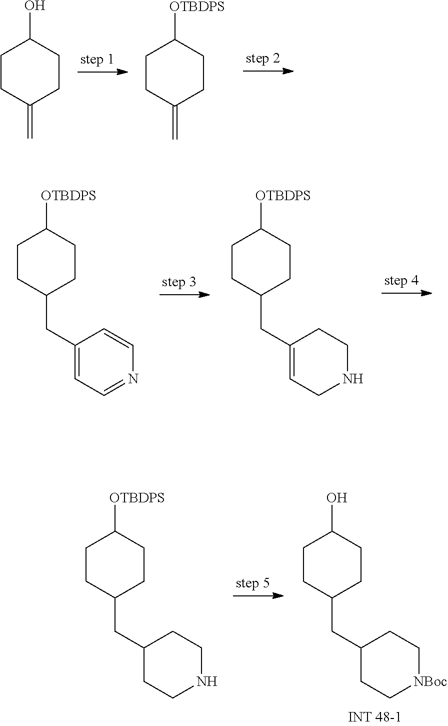

Step 1: tert-butyl-((4-methylenecyclohexyl)oxy)-diphenylsilane. To a solution of 4-methylenecyclohexanol (22.3 mmol) and imidazole (66.9 mmol) in acetonitrile (50 mL) was added tert-butylchlorodiphenylsilane (33.4 mmol) and the mixture was stirred at room temperature for 18 hours. The reaction mixture was then diluted with ethyl acetate, washed with saturated aqueous ammonium chloride, brine, dried over anhydrous sodium sulfate, filtered, and concentrated under reduced pressure to give a residue. The residue was purified by flash column chromatography on silica gel eluting with ethyl acetate/hexanes to afford the title compound. LCMS: 351.3.

Step 2: tert-butyl-diphenyl-[4-(4-pyridylmethyl)cyclohexoxyl]silane. A solution of 9-borabicyclo[3.3.1]nonane (0.5 M in THF, 23.2 mmol)) was added to tert-butyl ((4-methylenecyclohexyl)oxy)diphenylsilane (17.9 mmol) and the mixture was stirred at 80° C. for 4 hours under nitrogen. The resulting mixture was cooled to room temperature, followed by addition of N,N-dimethylformamide (50 mL), 10 mL degassed de-ionized water, 4-bromopyridine (21.4 mmol) and potassium carbonate (53.6 mmol) and the mixture was evacuated and backfilled with nitrogen (×3) followed by addition of (1,1′-bis(diphenylphosphino)ferrocene)-dichloropalladium(II) (1.79 mmol). The mixture was stirred at 80° C. for 18 h. The resulting mixture was cooled to room temperature and diluted with ethyl acetate, washed with saturated aqueous ammonium chloride, brine, dried over anhydrous sodium sulfate, filtered, and concentrated under reduced pressure to give a residue. The residue was purified by flash column chromatography on silica gel eluting with ethyl acetate/hexanes to afford the title compound as a mixture of cis/trans isomers. LCMS: 430.3.

Step 3: [4-[(1-benzyl-3,6-dihydro-2H-pyridin-4-yl)methyl]cyclohexoxyl]-tert-butyl-diphenyl-silane. To a solution of tert-butyl-diphenyl-[4-(4-pyridylmethyl)cyclohexoxyl]silane (13.3 mmol) in 50 mL dichloromethane was added benzyl bromide (39.8 mmol) and the mixture was stirred at room temperature for 18 h. The resulting mixture was concentrated under reduced pressure to give a residue which was triturated from hexanes repeatedly until no residual benzyl bromide remained. The resulting solid was dissolved in methanol (50 mL) and cooled to 0° C. followed by addition of sodium borohydride (39.8 mmol) portion wise and the resulting mixture was stirred at room temperature for 2 hours. The resulting mixture was diluted with ethyl acetate, washed with saturated aqueous ammonium chloride, brine, dried over anhydrous sodium sulfate, filtered, and concentrated under reduced pressure to give a residue. The residue was purified by flash column chromatography on silica gel eluting with ethyl acetate/hexanes to afford the title compound as a mixture of cis/trans isomers. LCMS: 524.4

Step 4: 4-((4-((tert-butyldiphenylsilyl)oxy)cyclohexyl)methyl)piperidine. [4-[(1-benzyl-3,6-dihydro-2H-pyridin-4-yl)methyl]cyclohexoxyl]-tert-butyl-diphenyl-silane (9.1 mmol) in anhydrous ethanol (50 mL) was added 10% palladium hydroxide on carbon (10 mmol) and the mixture was evacuated and backfilled with hydrogen (3×). The mixture was stirred at room temperature for 72 h. The resulting mixture was filtered over Celite followed by rinsing the celite layer with acetonitrile (3×). The filtrate was then concentrated under reduced pressure to give a residue which was used without further purification. LCMS: 436.4

Step 5: tert-butyl 4-[(4-hydroxycyclohexyl)methyl]piperidine-1-carboxylate (INT 48-1). To a solution of tert-butyl-diphenyl-[4-(4-piperidylmethyl)cyclohexoxyl]silane (8.9 mmol) and 4-dimethylaminopyridine (4.5 mmol) in acetonitrile (50 mL) at 0° C. was added di-tert-butylcarbonate (13 mmol) portionwise, and the mixture was allowed to warm to room temperature and stirred for 5 h. The resulting mixture was diluted with ethyl acetate, and the organic layer was washed with saturated aqueous ammonium chloride and brine, dried over anhydrous sodium sulfate, and concentrated under reduced pressure to give a residue, which was dissolved in tetrahydrofuran (50 mL) and treated with tetrabutylammonium fluoride (1.0 M in THF, 27 mmol).

The reaction mixture was stirred at room temperature for 18 h, then concentrated under reduced pressure to give a residue, which was purified by flash column chromatography on silica gel eluting with ethyl acetate/hexanes to afford the title compound as a mixture of cis/trans isomers. LCMS: 320.3.

### Intermediates 49-1-49-5.

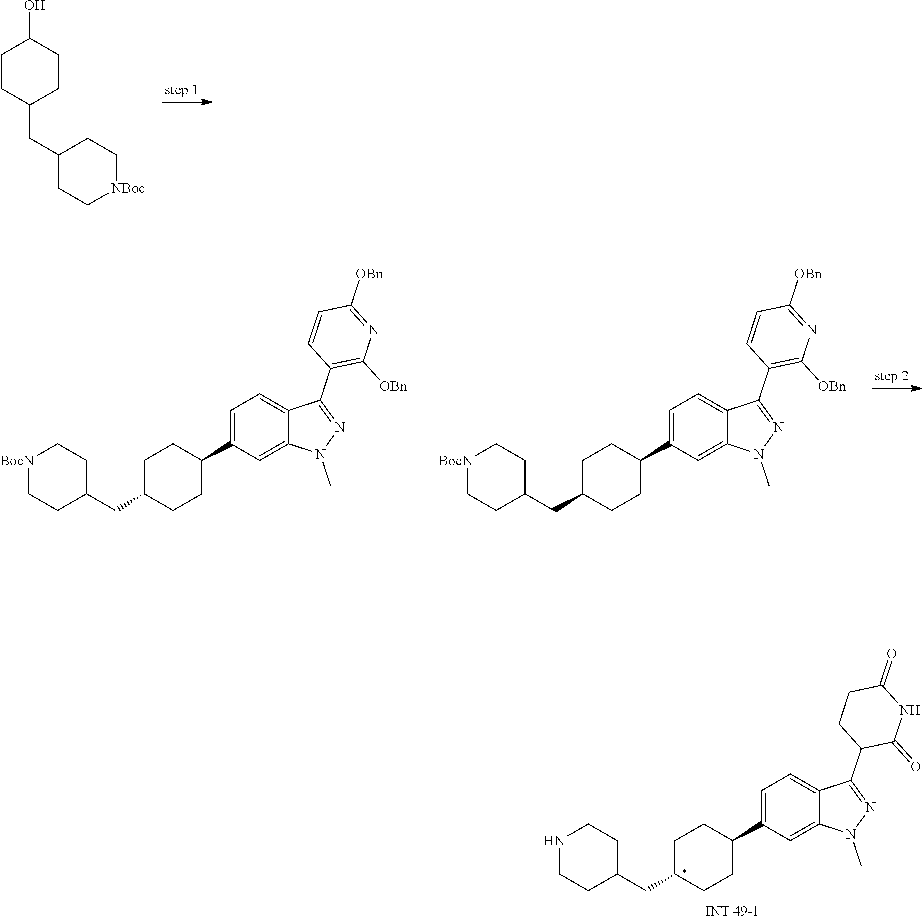 [SMILES: CC(C)(C)OC(=O)N1CCC(CC2CCC(O)CC2)CC1 ; Cn1nc(-c2ccc(OCc3ccccc3)nc2OCc2ccccc2)c2ccc([C@H]3CC[C@H](CC4CCN(C(=O)OC(C)(C)C)CC4)CC3)cc21 ; Cn1nc(-c2ccc(OCc3ccccc3)nc2OCc2ccccc2)c2ccc([C@H]3CC[C@@H](CC4CCN(C(=O)OC(C)(C)C)CC4)CC3)cc21 ; Cn1nc(C2CCC(=O)NC2=O)c2ccc([C@H]3CC[C@H](CC4CCNCC4)CC3)cc21]

Step 1: cis and trans-tert-butyl 4-[[4-[3-(2,6-dibenzyloxy-3-pyridyl)-1-methyl-indazol-6-yl]cyclohexyl]methyl]piperidine-1-carboxylate. To a solution of tert-butyl 4-[(4-hydroxycyclohexyl)methyl]piperidine-1-carboxylate (INT 48-1, 2.5 mmol) in tert-butyl methyl ether (7.5 mL) was added 5,7-di-tert-butyl-3-phenyl-1,3-benzoxazol-3-ium tetrafluoroborate (2.2 mmol), and the suspension was stirred vigorously for 5 minutes. Pyridine (2.2 mmol) was then added, and the mixture was stirred vigorously for an additional 15 minutes at room temperature. Separately, a solution of 6-bromo-3-(2,6-dibenzyloxy-3-pyridyl)-1-methyl-indazole (1.4 mmol), [4,4′-bis(1,1-dimethylethyl)-2,2′-bipyridine-N1,N1′]bis[2-(2-pyridinyl-N)phenyl-C]iridium(III) hexafluorophosphate (0.070 mmol), [4,4′-bis(tert-butyl)-2,2′-bipyridine]nickel dibromide (0.28 mmol), quinuclidine (2.5 mmol), and phthalimide (0.32 mmol) in N,N-dimethylacetamide (7.5 mL) was sparged with nitrogen gas for 5 minutes at room temperature. The suspension in tert-butyl methyl ether was added dropwise via syringe filter to the vial containing the catalyst mixture, the vial was sealed with parafilm, and irradiated at 450 nm for 18 hours. The resulting mixture was diluted with ethyl acetate, and the organic layer was washed with saturated aqueous ammonium chloride and brine, dried over anhydrous sodium sulfate, filtered, and concentrated under reduced pressure to yield a residue. The residue was purified by flash column chromatography on silica gel eluting with ethyl acetate/hexanes to afford the title compound as a mixture of cis/trans isomers. This mixture was further purified by prep-SFC to yield trans*-tert-butyl 4-[[4-[3-(2,6-dibenzyloxy-3-pyridyl)-1-methyl-indazol-6-yl]cyclohexyl]methyl]piperidine-1-carboxylate (peak 1), LCMS: 423.3, and cis*-tert-butyl 4-[[4-[3-(2,6-dibenzyloxy-3-pyridyl)-1-methyl-indazol-6-yl]cyclohexyl]methyl]piperidine-1-carboxylate (Peak 2), LCMS: 423.3. The relative stereochemistry was arbitrarily assigned.

Step 2: trans*-3-[1-methyl-6-[4-(4-piperidylmethyl)cyclohexyl]indazol-3-yl]piperidine-2,6-dione trifluoroacetic acid (INT 49-2). To a solution of trans* tert-butyl 4-[[4-[3-(2,6-dibenzyloxy-3-pyridyl)-1-methyl-indazol-6-yl]cyclohexyl]methyl]piperidine-1-carboxylate (Peak 1, 0.51 mmol) in anhydrous ethanol (10 mL) was added 10% palladium hydroxide on carbon (0.56 mmol), and the mixture was evacuated and backfilled with hydrogen three times. The mixture was stirred at room temperature for 48 hours, then filtered over Celite, and filtrate was rinsed with acetonitrile three times. The filtrate was concentrated under reduced pressure to give a residue, which was used without further purification. This residue was dissolved in dichloromethane (10 mL), followed by addition of trifluoroacetic acid (10.2 mmol), and the mixture was stirred at room temperature for 2 hours. The resulting mixture was diluted with ethyl acetate and concentrated under reduced pressure; this process was repeated three times to give a residue, which was used without further purification. LCMS: 423.3.

The following intermediates were made in a similar fashion to INT 49-1 and are shown below in Table 2Z. To prepare the below intermediates, different reagents/starting materials were used than previously described in the steps toward INT 49-1 and are noted in the last column of Table 2Z—“Changes to Procedure: Different Reagents/Starting Materials”. A person of ordinary skill in the art will readily recognize which reagents/starting materials used for the synthesis of INT 49-1 were replaced with the different reagents/starting materials noted

TABLE 2Z
Intermediates

| | | | Changes to Procedure: Different |
|---|---|---|---|
| Intermediate | Structure | LCMS | Reagents/Starting Materials |
| INT 49-2 | 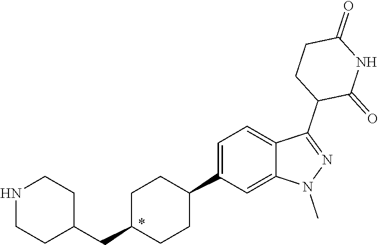 [SMILES: Cn1nc(C2CCC(=O)NC2=O)c2ccc([C@H]3CC[C@@H](CC4CCNCC4)CC3)cc21] | 423.3 | Peak 2 was isolated in step 1 and carried forward. |
| INT 49-3 | 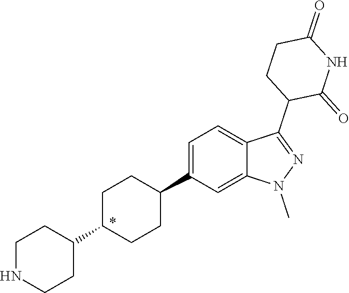 [SMILES: Cn1nc(C2CCC(=O)NC2=O)c2ccc([C@H]3CC[C@H](C4CCNCC4)CC3)cc21] | 409.3 | tert-butyl 4-(4- hydroxycyclohexyl)piperidine-1- carboxylate in step 1. Peak 1 was isolated in step 1 and carried forward. |
| INT 49-4 | 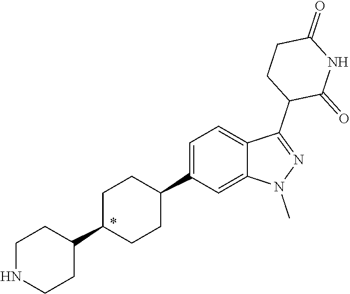 [SMILES: Cn1nc(C2CCC(=O)NC2=O)c2ccc([C@H]3CC[C@@H](C4CCNCC4)CC3)cc21] | 409.3 | tert-butyl 4-(4- hydroxycyclohexyl)piperidine-1- carboxylate in step 1. Peak 2 was isolated in step 1 and carried forward. |
| INT 49-5 | 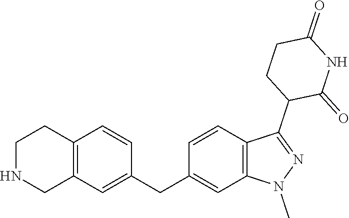 [SMILES: Cn1nc(C2CCC(=O)NC2=O)c2ccc(Cc3ccc4c(c3)CNCC4)cc21] | 389.2 | tert-butyl 7-(hydroxymethyl)-3,4- dihydro-1H-isoquinoline-2-carboxylate in step 1. |

### Intermediates 50-1-50-2.

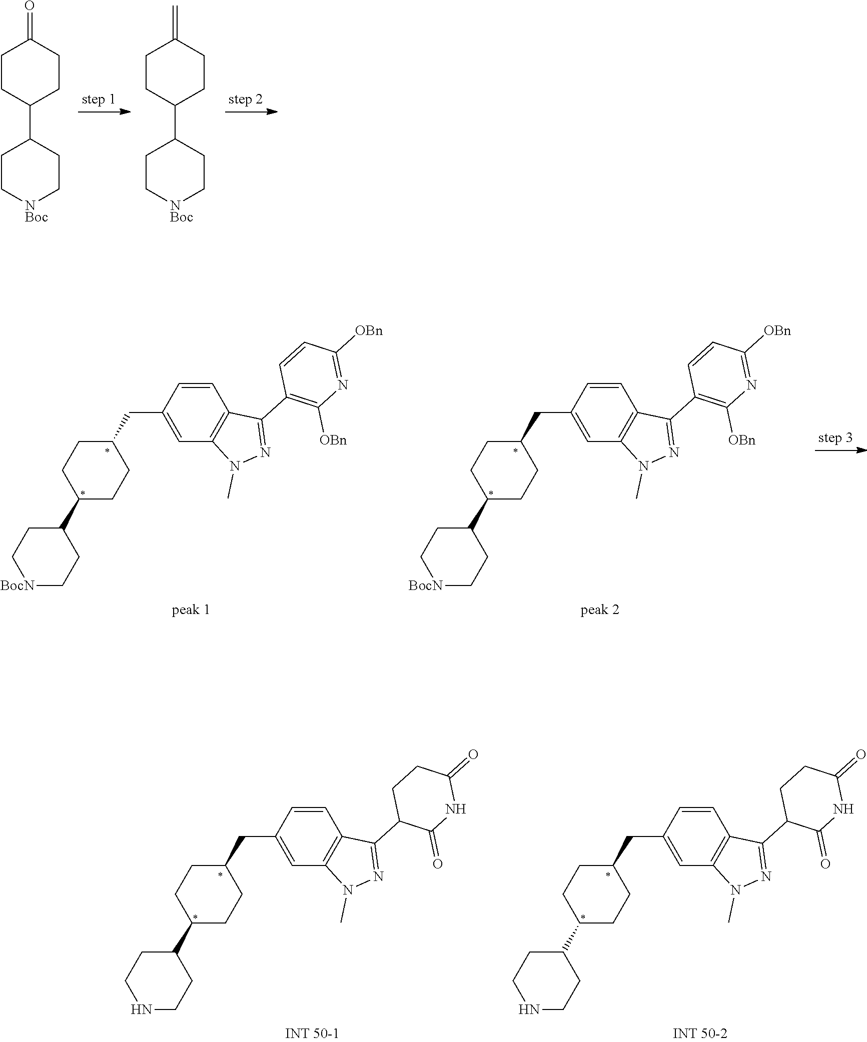 [SMILES: CC(C)(C)OC(=O)N1CCC(C2CCC(=O)CC2)CC1 ; C=C1CCC(C2CCN(C(=O)OC(C)(C)C)CC2)CC1 ; Cn1nc(-c2ccc(OCc3ccccc3)nc2OCc2ccccc2)c2ccc(C[C@H]3CC[C@H](C4CCN(C(=O)OC(C)(C)C)CC4)CC3)cc21 ; Cn1nc(-c2ccc(OCc3ccccc3)nc2OCc2ccccc2)c2ccc(C[C@H]3CC[C@@H](C4CCN(C(=O)OC(C)(C)C)CC4)CC3)cc21 ; Cn1nc(C2CCC(=O)NC2=O)c2ccc(C[C@H]3CC[C@@H](C4CCNCC4)CC3)cc21 ; Cn1nc(C2CCC(=O)NC2=O)c2ccc(C[C@H]3CC[C@H](C4CCNCC4)CC3)cc21]

Step 1: tert-butyl 4-(4-methylenecyclohexyl)piperidine-1-carboxylate. To a solution of methyltriphenylphosphonium bromide (3.0 mmol) in DMSO (5.0 mL) was added potassium tert-butoxide (3.0 mmol). The reaction mixture was stirred at room temperature for 1 hour, after which tert-butyl 4-(4-oxocyclohexyl)piperidine-1-carboxylate (1.78 mmol) was added. Stirring was continued at room temperature for 18 hours. The reaction mixture was then slowly diluted with water and extracted with ethyl acetate (3×). The combined organic layers were washed with brine, dried over anhydrous sodium sulfate, and concentrated under reduced pressure to yield a crude residue. The residue was purified by flash column chromatography on silica gel, eluting with ethyl acetate/hexanes, to afford the title compound. LCMS: 224.2.

Step 2. trans- and cis-tert-butyl 4-[4-[[3-(2,6-dibenzyloxy-3-pyridyl)-1-methyl-indazol-6-yl]methyl]cyclohexyl]piperidine-1-carboxylate. An oven-dried vial was charged with tert-butyl 4-(4-methylenecyclohexyl)piperidine-1-carboxylate (0.87 mmol), degassed, and backfilled with nitrogen. The vial was cooled to 0° C., followed by the addition of a solution of 9-borabicyclo(3.3.1)nonane in THE (0.5 M, 2.3 mL). The reaction mixture was stirred at room temperature for 3 hours. Degassed DMF (3.0 mL), degassed water (1.0 mL), potassium carbonate (2.6 mmol), 6-bromo-3-(2,6-dibenzyloxy-3-pyridyl)-1-methylindazole (1.1 mmol), and (1,1′-bis(diphenylphosphino)ferrocene)palladium(II) dichloride (0.18 mmol) were then added sequentially. The mixture was stirred under a nitrogen atmosphere at 70° C. for 18 hours. After cooling to room temperature, the reaction mixture was diluted with ethyl acetate. The organic layer was washed with saturated aqueous ammonium chloride and brine, dried over anhydrous sodium sulfate, and concentrated under reduced pressure to yield a crude residue. The residue was purified by flash column chromatography on silica gel, eluting with ethyl acetate/hexanes, to afford the title compound as a mixture of cis/trans isomers. This mixture was further purified by prep-SFC to yield trans*-tert-butyl 4-[4-[[3-(2,6-dibenzyloxy-3-pyridyl)-1-methyl-indazol-6-yl]methyl]cyclohexyl]piperidine-1-carboxylate (peak 1) and cis*-tert-butyl 4-[4-[[3-(2,6-dibenzyloxy-3-pyridyl)-1-methyl-indazol-6-yl]methyl]cyclohexyl]piperidine-1-carboxylate (peak 2). The relative stereochemistry was arbitrarily assigned.

Step 3: cis*-3-[1-methyl-6-[[4-(4-piperidyl)cyclohexyl]methyl]indazol-3-yl]piperidine-2,6-dione (INT 50-1). To a solution of cis*-tert-butyl 4-[4-[[3-(2,6-dibenzyloxy-3-pyridyl)-1-methyl-indazol-6-yl]methyl]cyclohexyl]piperidine-1-carboxylate (peak 2, 0.15 mmol) in anhydrous ethanol (5.0 mL) was added 10% palladium hydroxide on carbon (0.15 mmol), and the mixture was evacuated and backfilled with hydrogen (3×). The mixture was stirred at room temperature for 18 hours, then filtered over Celite, and rinsed with acetonitrile (3×). The filtrate was concentrated under reduced pressure to give a residue, which was used without further purification. This residue was dissolved in dichloromethane (3.0 mL), followed by addition of trifluoroacetic acid (2.8 mmol), and the mixture was stirred at room temperature for 2 hours. The resulting mixture was diluted with ethyl acetate and concentrated under reduced pressure; this process was repeated three times to give a residue, which was used without further purification. LCMS: 423.3

trans*-3-[1-methyl-6-[[4-(4-piperidyl)cyclohexyl]methyl]indazol-3-yl]piperidine-2,6-dione (INT 50-2) was prepared using trans*-tert-butyl 4-[4-[[3-(2,6-dibenzyloxy-3-pyridyl)-1-methyl-indazol-6-yl]methyl]cyclohexyl]piperidine-1-carboxylate (peak 1), isolated in step 2. LCMS: 423.3.

### Examples 1-1-1-49

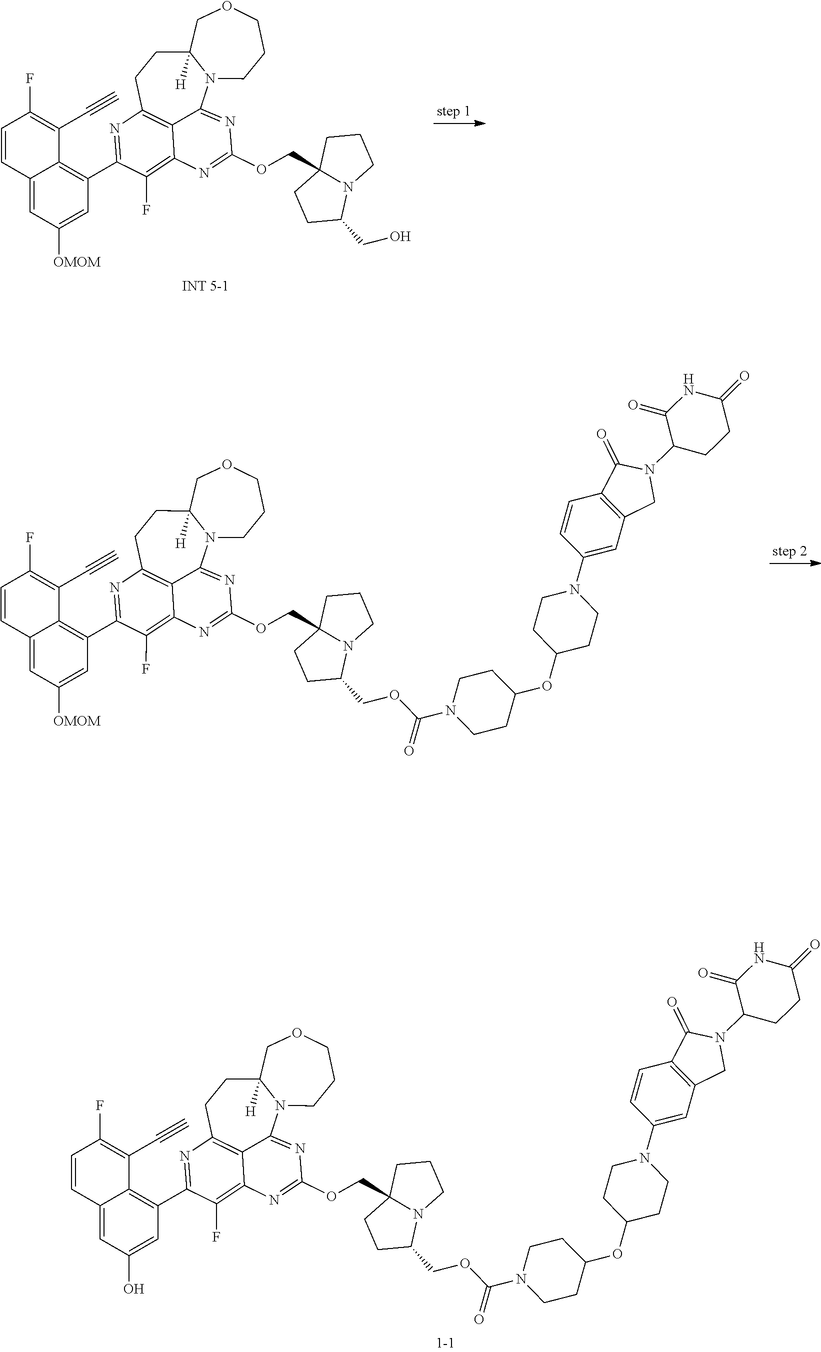

Step 1: ((3S,7aS)-7a-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(2-(2,6-dioxopiperidin-3-yl)-1-oxoisoindolin-5-yl)piperidin-4-yl)oxy)piperidine-1-carboxylate. To a solution of INT 5-1 (0.03 mmol) in tetrahydrofuran (1 mL) at 0° C. was added 4-nitrophenyl chloroformate (0.04 mmol) and diisopropylethylamine (0.1 mmol). The resulting mixture was stirred at 0° C. for 1 hour under an argon atmosphere. To the reaction mixture was added a solution of 3-(1-oxo-5-(4-(piperidin-4-yloxy)piperidin-1-yl)isoindolin-2-yl)piperidine-2,6-dione (INT 9-1) (0.08 mmol) and diisopropylethylamine (0.2 mmol) in dimethyl sulfoxide (1 mL) and the mixture was warmed to room temperature and stirred for 1 hour. The mixture was then diluted with water (4.0 mL) and extracted with 4:1 dichloromethane/isopropanol (3×10 mL). The organic layer was then washed with brine (20 mL), dried over anhydrous magnesium sulfate, filtered, and concentrated under reduced pressure. The residue was purified by flash column chromatography on silica gel eluting with 10% ammonium hydroxide in ethanol/dichloromethane. Fractions containing the product were pooled and concentrated under reduced pressure to yield the title compound. LCMS: 1124.5.

Step 2: ((3S,7aS)-7a-((((S)-2-(8-ethynyl-7-fluoro-3-hydroxynaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(2-(2,6-dioxopiperidin-3-yl)-1-oxoisoindolin-5-yl)piperidin-4-yl)oxy)piperidine-1-carboxylate (Example 1-1). To a solution of ((3S,7aS)-7a-((((S)-2-(8-ethynyl-7-fluoro-3-(methoxymethoxy)naphthalen-1-yl)-1-fluoro-4,5,5a,6,9,10-hexahydro-8H-7-oxa-3,10a,11,13-tetraazanaphtho[l1,8-ab]heptalen-12-yl)oxy)methyl)hexahydro-1H-pyrrolizin-3-yl)methyl 4-((1-(2-(2,6-dioxopiperidin-3-yl)-1-oxoisoindolin-5-yl)piperidin-4-yl)oxy)piperidine-1-carboxylate (0.03 mmol) in dichloromethane (1 mL) was added hydrochloric acid in 1,4-dioxane (4.0 M, 0.5 mL) at room temperature for 1 hour. Upon completion, the reaction was concentrated and purified by RP-HPLC (0 to 90%, 0.1% trifluoroacetic acid in acetonitrile/0.1% trifluoroacetic acid in water). Fractions containing the product were pooled and lyophilized to yield the title compound. ¹H NMR (400 MHz, Methanol-d₄) δ 7.88 (dd, J=9.2, 5.7 Hz, 1H), 7.63 (d, J=8.7 Hz, 1H), 7.39-7.31 (m, 2H), 7.26 (dd, J=14.6, 2.6 Hz, 1H), 7.09 (d, J=8.1 Hz, 2H), 5.10 (dd, J=13.3, 5.1 Hz, 1H), 4.76 (dd, J=12.5, 3.6 Hz, 2H), 4.63 (d, J=12.6 Hz, 1H), 4.60-4.46 (m, 2H), 4.45-4.30 (m, 3H), 4.26 (s, 1H), 4.14-3.89 (m, 4H), 3.89-3.54 (m, 9H), 3.48 (p, J=1.7 Hz, 1H), 3.26-3.01 (m, 6H), 2.90 (ddd, J=18.5, 13.4, 5.4 Hz, 2H), 2.82-2.72 (m, 1H), 2.55-1.88 (m, 15H), 1.81 (s, 2H), 1.71-1.41 (m, 4H). LCMS: 1080.9.

The following Examples were made in a similar fashion to Example 1-1 and are shown below in Table 3A1. To prepare the below Examples, different reagents/starting materials were used than some of those described in the steps toward Example 1-1 and are noted in the last column of Table 3A2—“Changes to Procedure: Different Reagents/Starting Materials”. A person of ordinary skill in the art will readily recognize which reagents/starting materials used for the synthesis of Example 1-1 were replaced with the different reagents/starting materials noted below.

TABLE 3A1
Compounds

| Example | Structure |
|---|---|
| 1-2 | 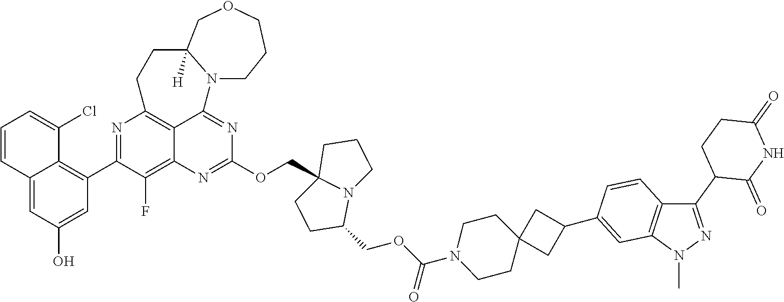 |
| 1-3 | 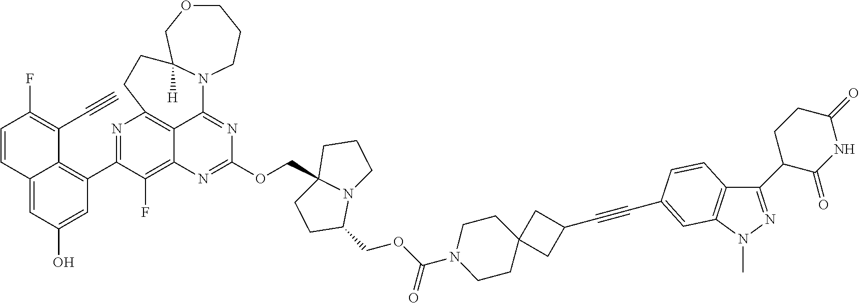 |
| 1-4 | 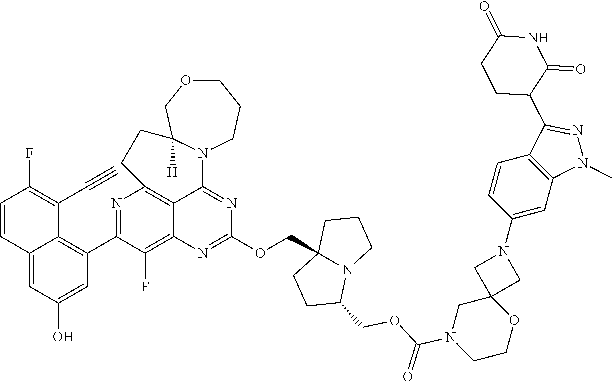 |
| 1-5 | 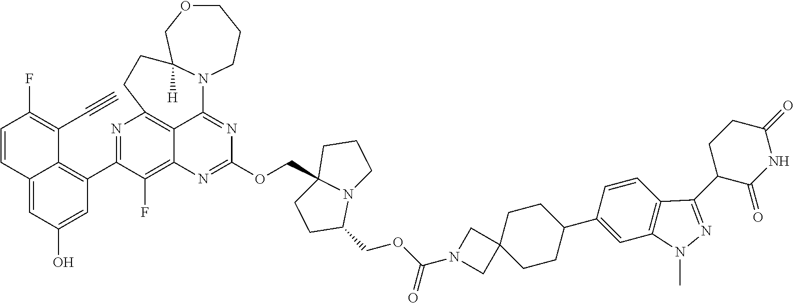 |
| 1-6 | 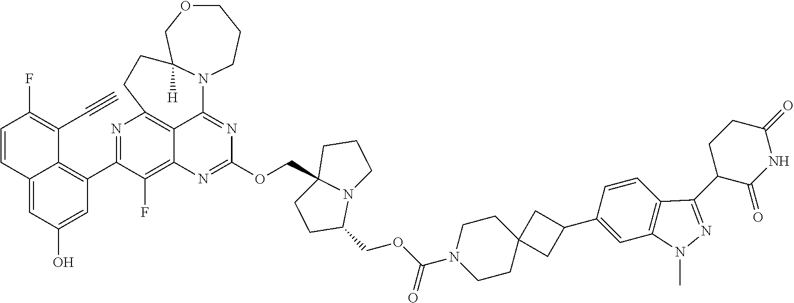 |
| 1-7 | 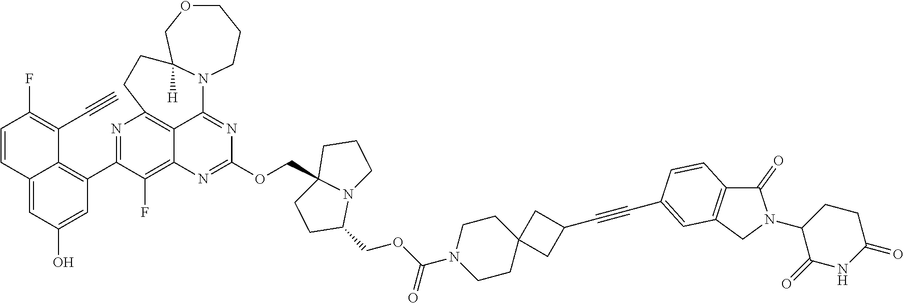 |
| 1-8 | 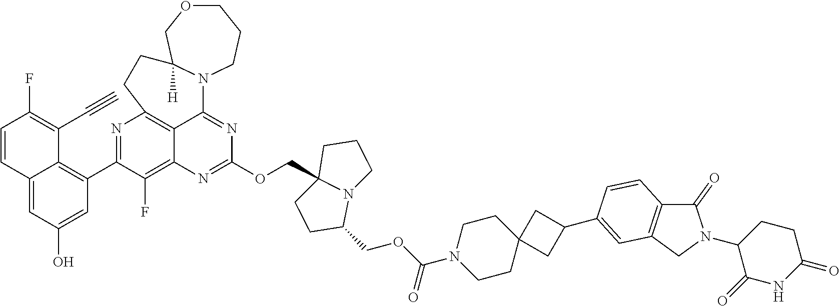 |
| 1-9 | 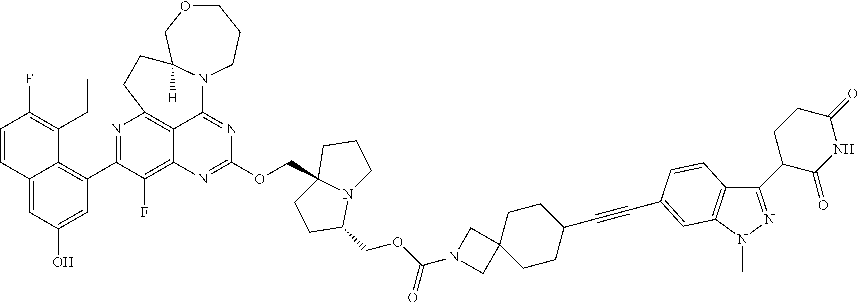 |
| 1-10 | 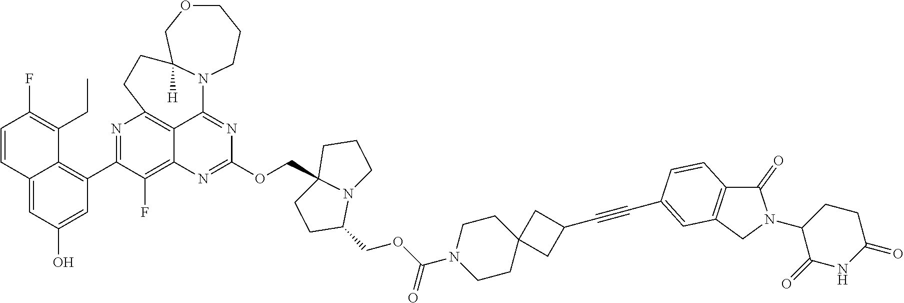 |
| 1-11 | 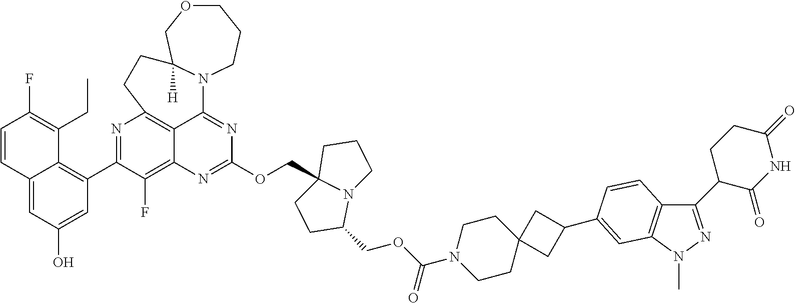 |
| 1-12 | 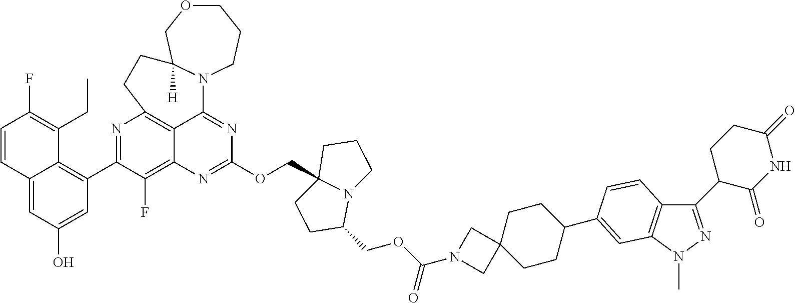 |
| 1-13 | 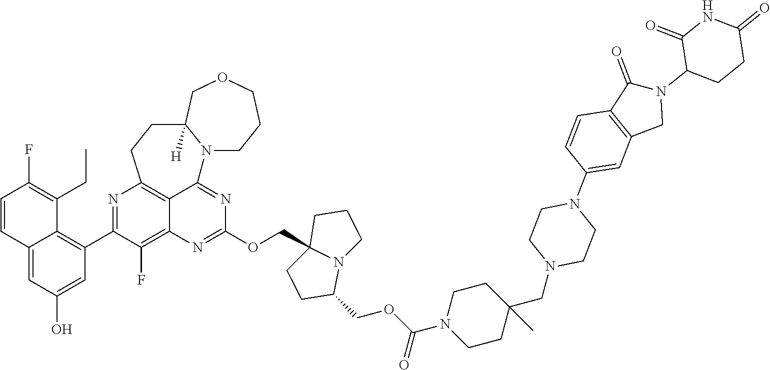 |
| 1-14 | 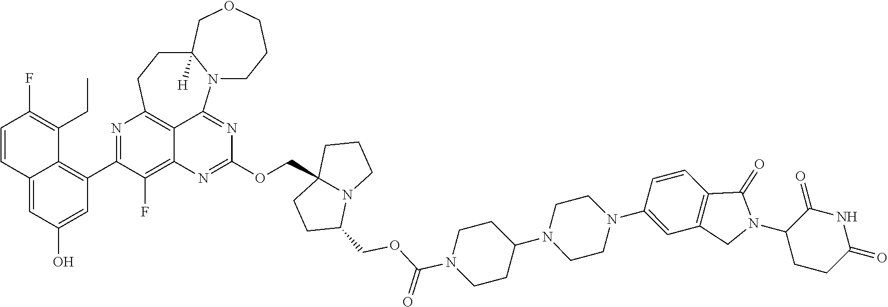 |
| 1-15 | 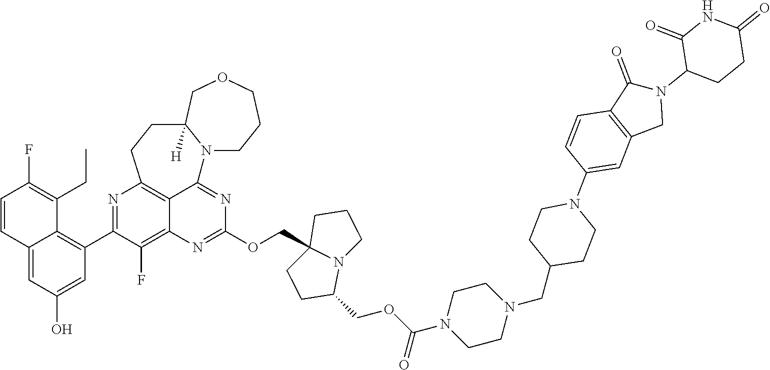 |
| 1-16 | 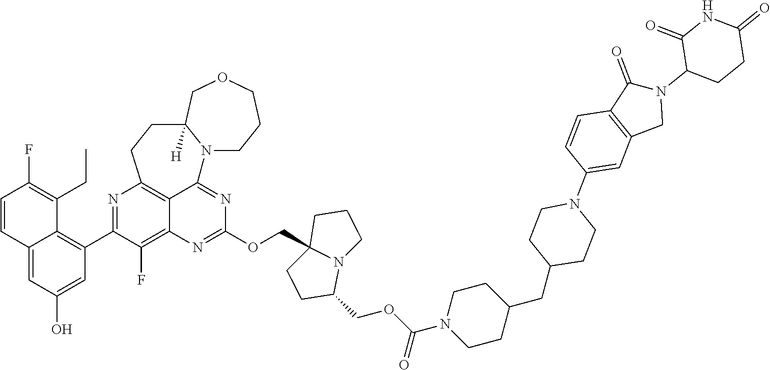 |
| 1-17 | 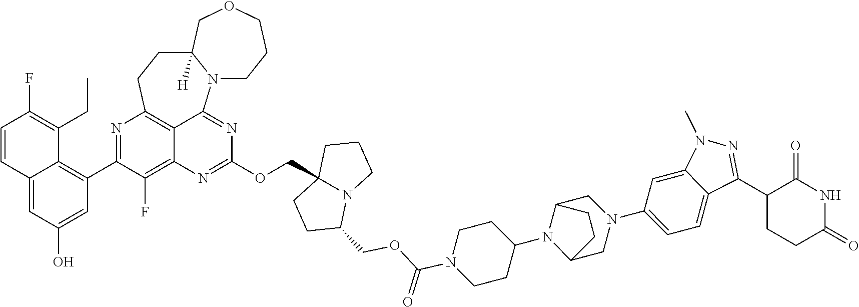 |
| 1-18 | 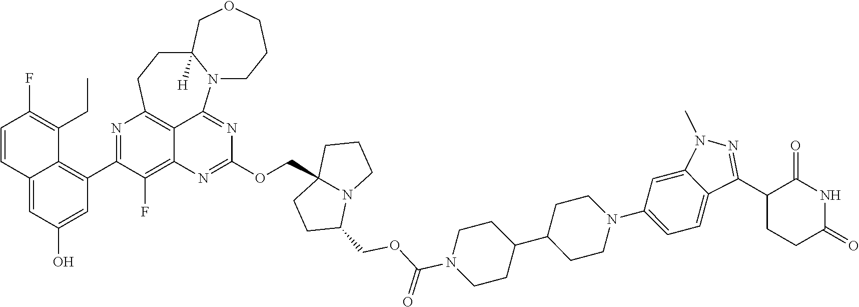 |
| 1-19 | 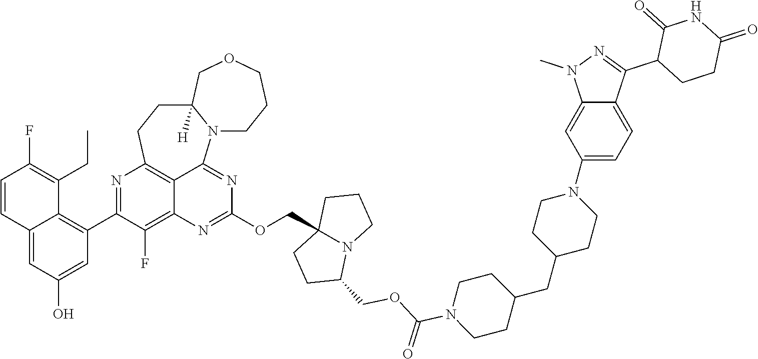 |
| 1-20 | 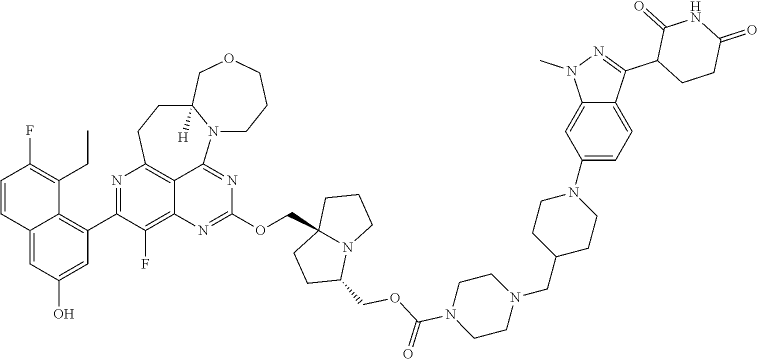 |
| 1-21 | 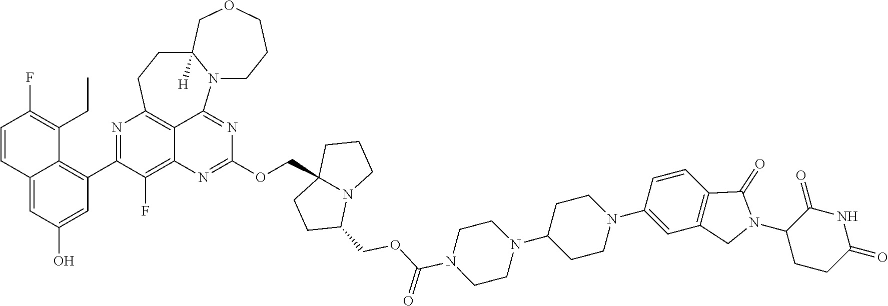 |
| 1-22 | 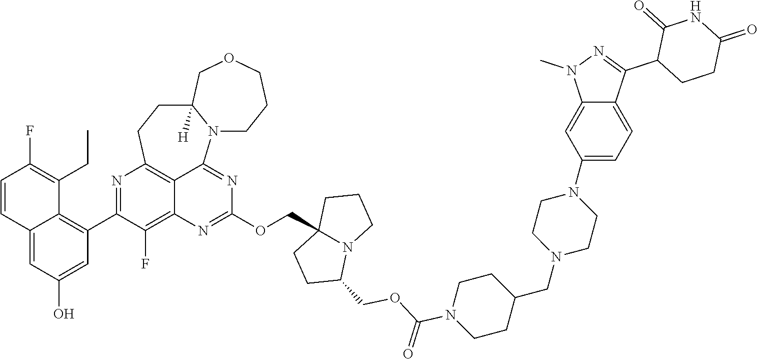 |
| 1-23 | 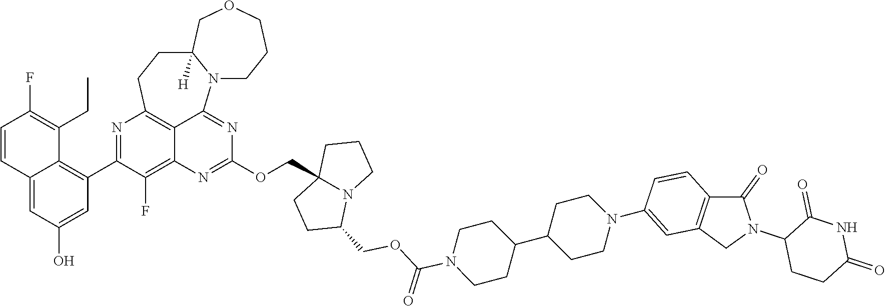 |
| 1-24 | 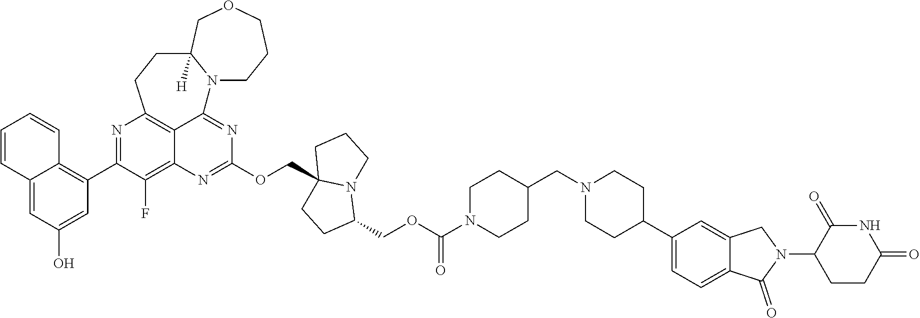 |
| 1-25 | 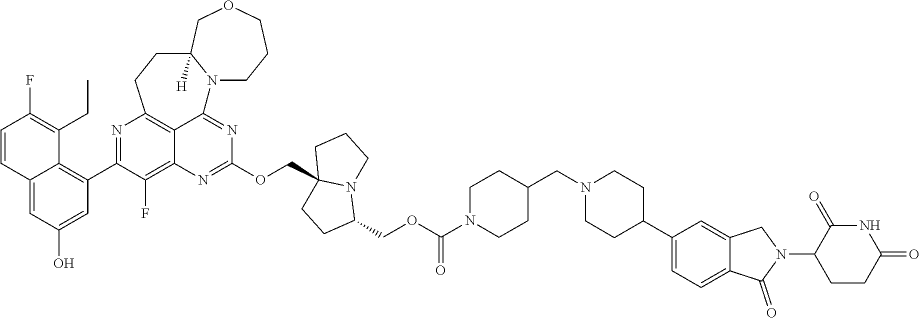 |
| 1-26 | 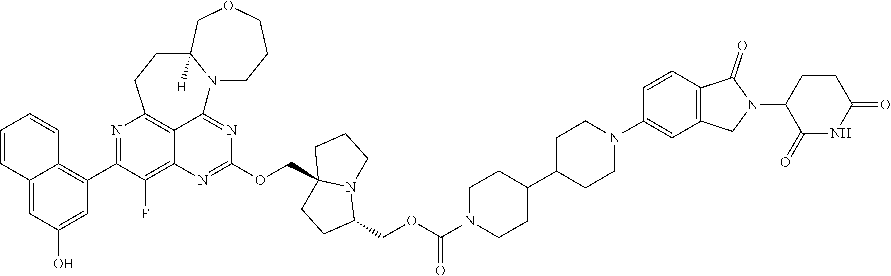 |
| 1-27 | 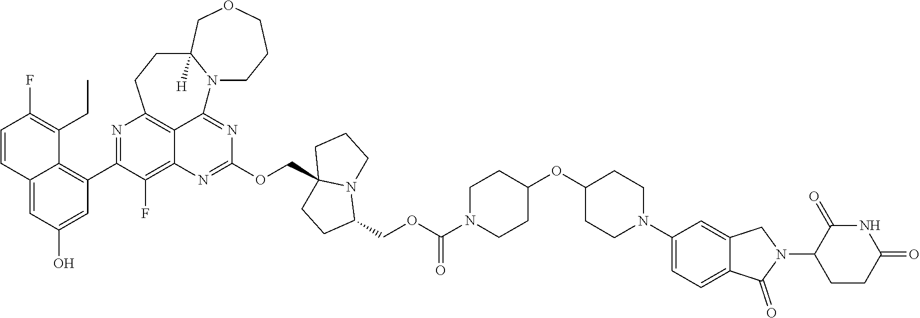 |
| 1-28 | 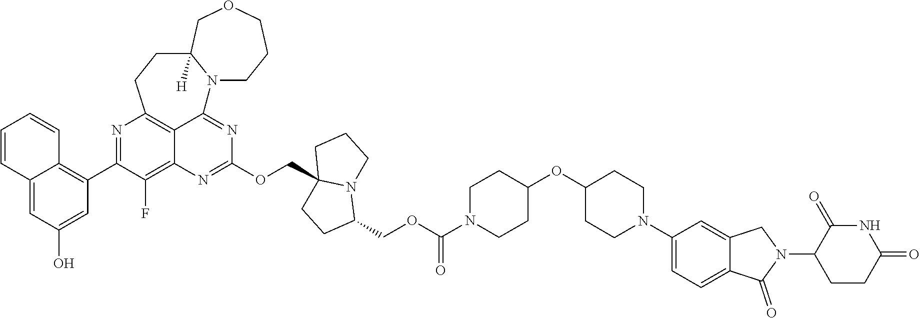 |
| 1-29 | 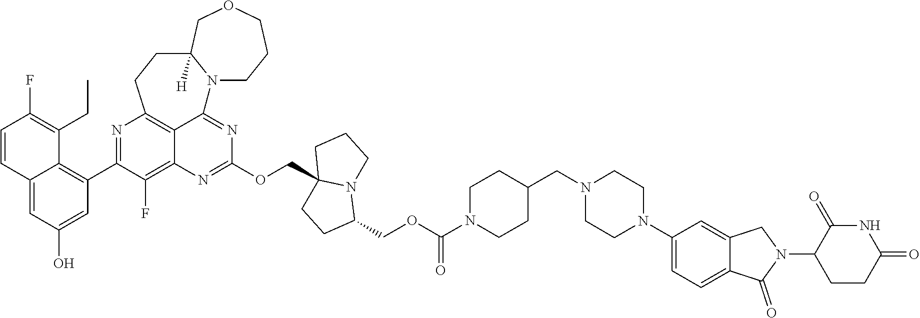 |
| 1-30 | 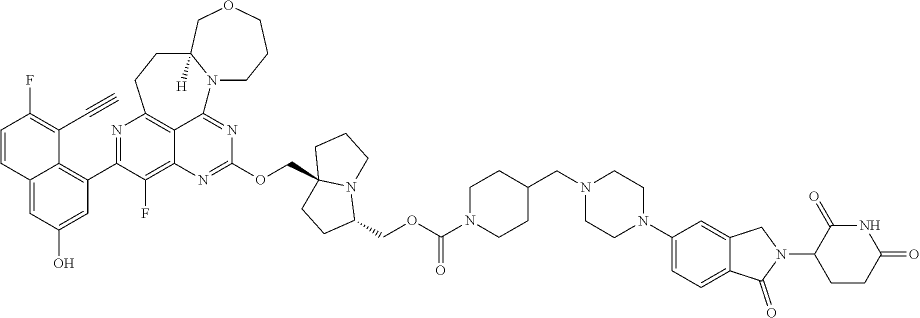 |
| 1-31 | 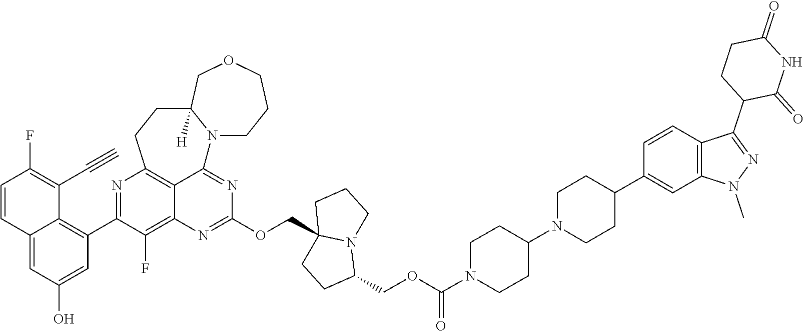 |
| 1-32 | 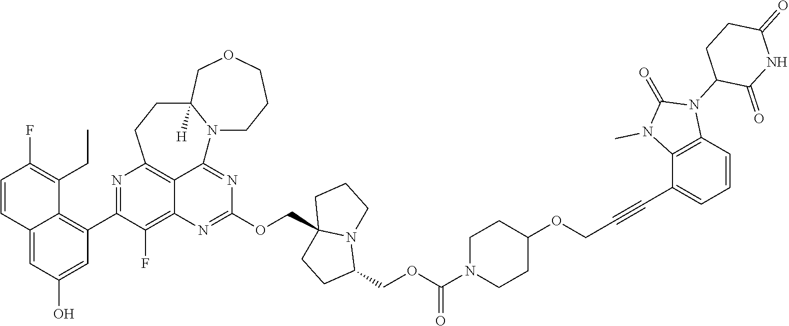 |
| 1-33 | 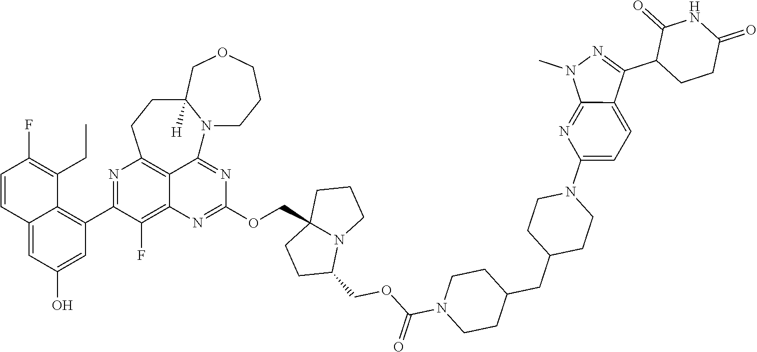 |
| 1-34 | 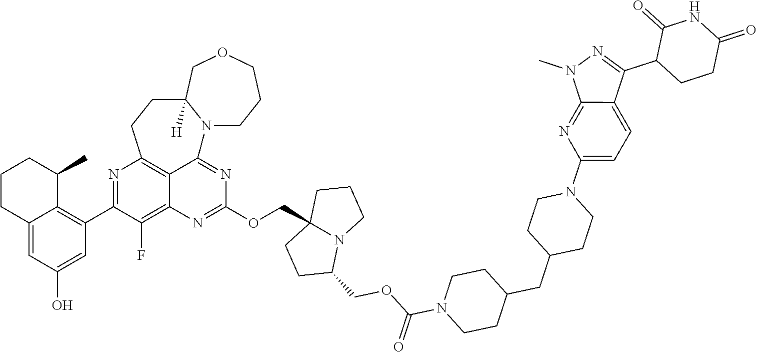 |
| 1-35 | 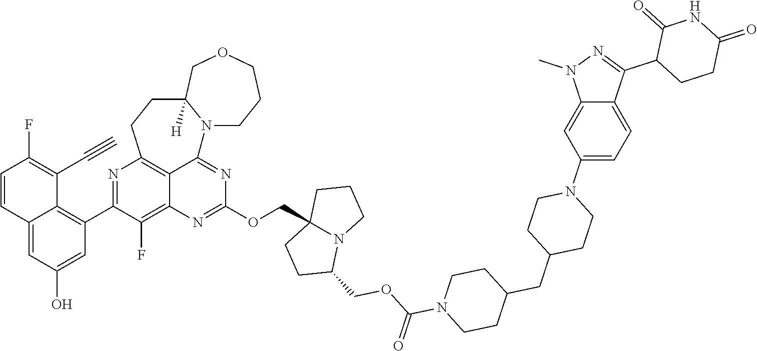 |
| 1-36 | 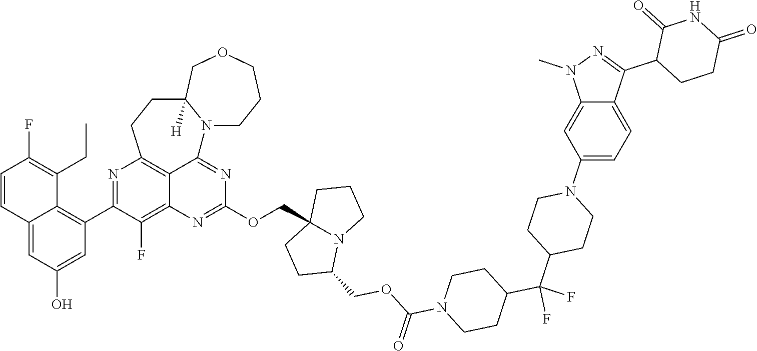 |
| 1-37 | 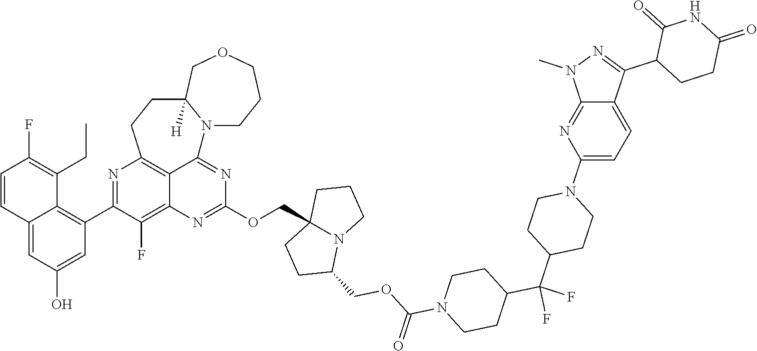 |
| 1-38 | 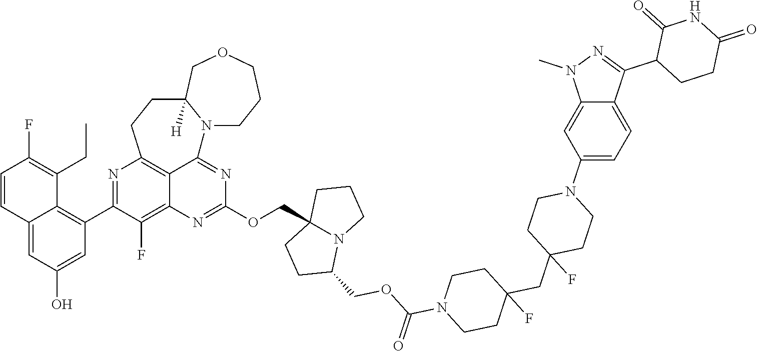 |
| 1-39 | 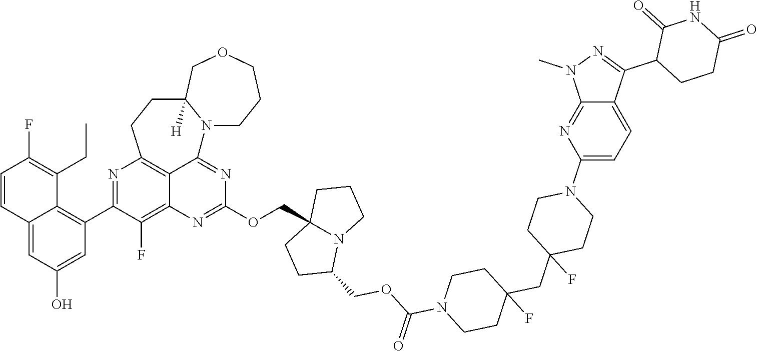 |
| 1-40 | 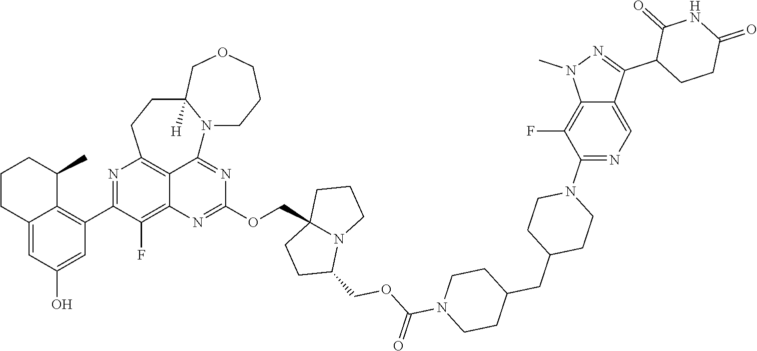 |
| 1-41 | 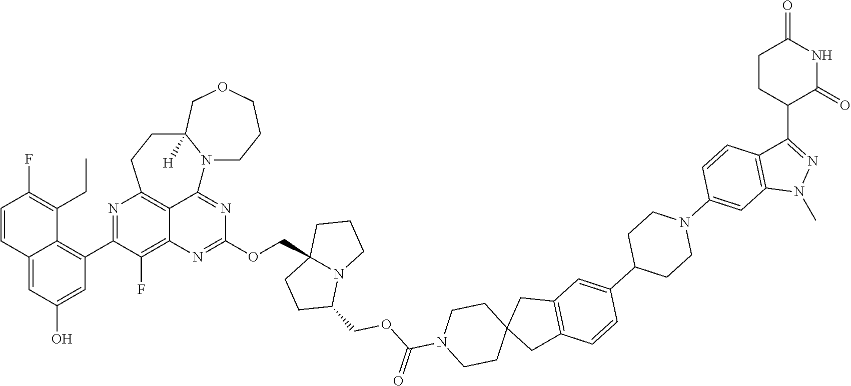 |
| 1-42 | 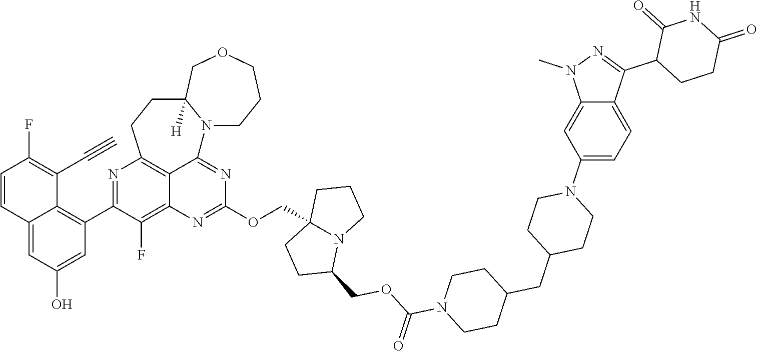 |
| 1-43 | 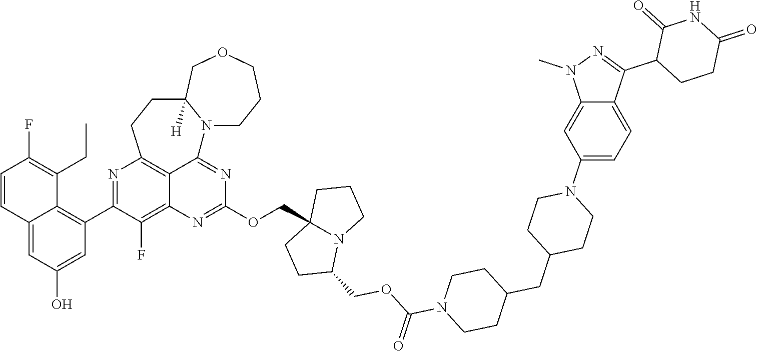 |
| 1-44 | 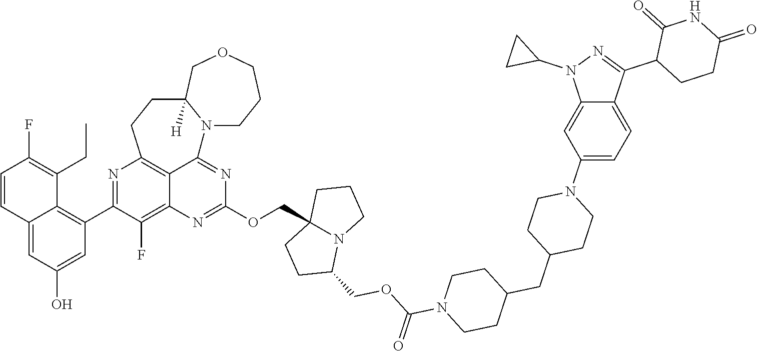 |
| 1-45 | 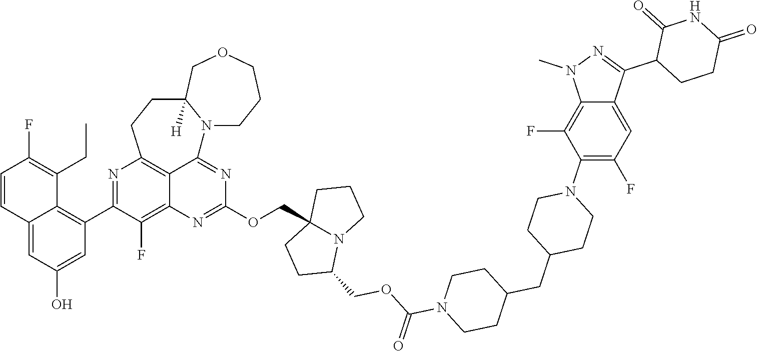 |
| 1-46 | 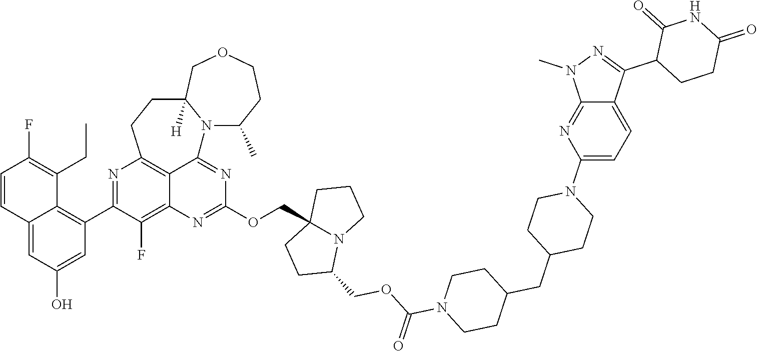 |
| 1-47 | 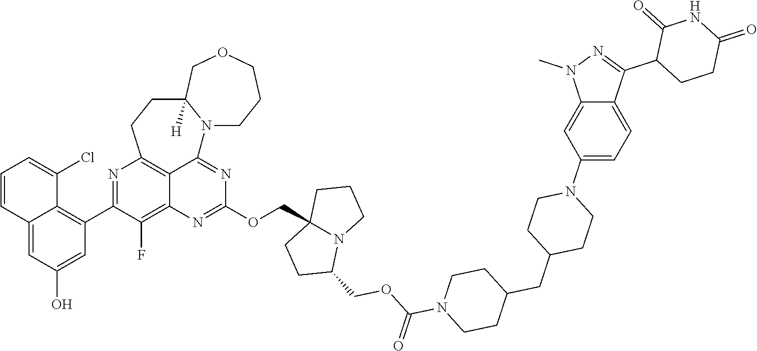 |
| 1-48 | 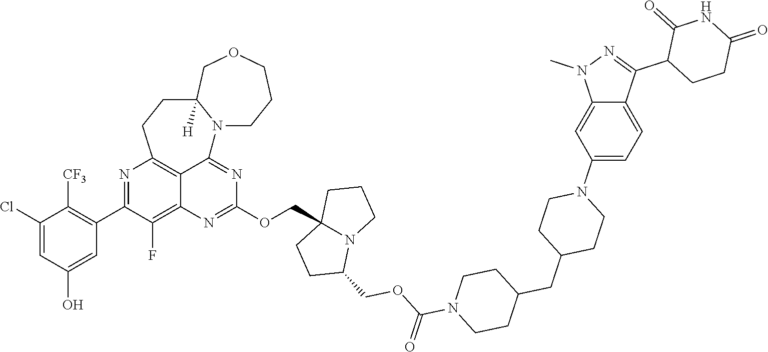 |
| 1-49 | 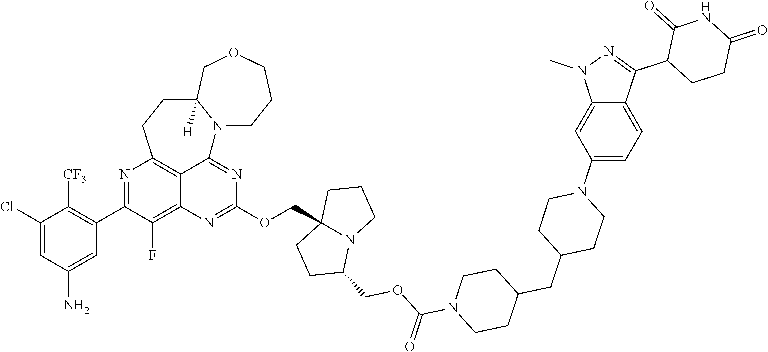 |

TABLE 3A2
Compounds

| | | | Changes to |
|---|---|---|---|
| | | | Procedure: |
| | | | Different |
| | | | Reagents/ |
| | | | Starting |
| Example | LCMS | ¹H-NMR (400 MHz, Methanol-d₄) | Materials |
| 1-2 | 1011.9 | δ 7.72 (s, 1H), 7.64 (dd, J = 8.4, 2.4 Hz, 1H), 7.41- | INT 5-3 and |
| | | 7.13 (m, 5H), 7.04 (d, J = 8.4 Hz, 1H), 4.79-4.46 (m, | INT 7-1 |
| | | 5H), 4.42-4.18 (m, 3H), 4.14-3.85 (m, 7H), 3.80- | in step 1 |
| | | 3.54 (m, 4H), 3.56-3.44 (m, 4H), 3.23-2.97 (m, 3H), | |
| | | 2.85-2.64 (m, 2H), 2.54-2.21 (m, 10H), 2.22-2.08 | |
| | | (m, 4H), 2.03-1.92 (m, 3H), 1.83-1.45 (m, 4H). | |
| 1-3 | 1043.9 | δ 7.97-7.82 (m, 1H), 7.72-7.62 (m, 1H), 7.55 (s, | INT 8-2 |
| | | 1H), 7.46-7.24 (m, 3H), 7.17-7.08 (m, 1H), 4.82- | in step 1 |
| | | 4.65 (m, 2H), 4.64-4.45 (m, 2H), 4.44-4.21 (m, 3H), | |
| | | 4.19-3.81 (m, 7H), 3.80-3.57 (m, 3H), 3.54-2.98 | |
| | | (m, 12H), 2.91-2.66 (m, 2H), 2.59-1.86 (m, 15H), | |
| | | 1.77-1.48 (m, 4H). | |
| 1-4 | 1022.9 | δ 7.98-7.80 (m, 1H), 7.59-7.20 (m, 4H), 6.52-6.30 | INT 6-3 |
| | | (m, 2H), 4.80-4.20 (m, 8H), 4.17-3.39 (m, 19H), | in step 1 |
| | | 3.23-2.98 (m, 3H), 2.91-2.64 (m, 3H), 2.55-1.81 | |
| | | (m, 14H). | |
| 1-5 | 1020.5 | δ 7.95-7.79 (m, 1H), 7.68-7.54 (m, 1H), 7.50-6.90 | INT 7-2 |
| | | (m, 5H), 4.85-4.21 (m, 8H), 4.20-3.55 (m, 13H), | in step 1 |
| | | 3.54-3.37 (m, 2H), 3.28-2.98 (m, 3H), 2.97-2.59 | |
| | | (m, 4H), 2.53-1.28 (m, 20H). | |
| 1-6 | 1020.5 | δ 7.96-7.75 (m, 1H), 7.65 (dd, J = 8.5, 4.3 Hz, 1H), | INT 7-1 |
| | | 7.47-7.23 (m, 4H), 7.05 (d, J = 8.5 Hz, 1H), 4.74 (q, | in step 1 |
| | | J = 12.4 Hz, 2H), 4.65-4.44 (m, 2H), 4.45-4.22 (m, | |
| | | 3H), 4.20-3.81 (m, 7H), 3.81-3.58 (m, 4H), 3.57- | |
| | | 3.34 (m, 6H), 3.27-3.01 (m, 3H), 2.91-2.66 (m, 2H), | |
| | | 2.59-1.84 (m, 17H), 1.84-1.66 (m, 2H), 1.64-1.39 | |
| | | (m, 2H). | |
| 1-7 | 1045.5 | δ 7.97-7.84 (m, 1H), 7.80-7.68 (m, 1H), 7.63-7.45 | INT 12-1 |
| | | (m, 2H), 7.44-7.26 (m, 3H), 5.16 (dd, J = 13.1, 5.0 | in step 1 |
| | | Hz, 1H), 4.82-4.66 (m, 2H), 4.65-4.22 (m, 6H), | |
| | | 4.19-3.81 (m, 5H), 3.78-3.67 (m, 2H), 3.65-3.55 (m, | |
| | | 1H), 3.54-3.01 (m, 9H), 3.00-2.86 (m, 1H), 2.84- | |
| | | 2.75 (m, 1H), 2.59-1.88 (m, 17H), 1.75-1.48 (m, | |
| | | 4H). | |
| 1-8 | 1020.9 | δ 7.95-7.71 (m, 2H), 7.54-7.21 (m, 5H), 5.17 (dd, | INT 13-1 |
| | | J = 13.3, 5.2 Hz, 1H), 4.83-4.20 (m, 7H), 4.19-3.57 | in step 1 |
| | | (m, 8H), 3.57-3.41 (m, 3H), 3.33 (m, 14H), 3.28- | |
| | | 2.69 (m, 3H), 2.62-1.60 (m, 11H), 1.59-1.39 (m, | |
| | | 2H). | |
| 1-9 | 1047.9 | δ 7.75-7.61 (m, 2H), 7.59-7.48 (m, 1H), 7.37-7.17 | INT 5-4 and |
| | | (m, 2H), 7.16-7.02 (m, 2H), 4.82-4.47 (m, 5H), 4.38 | INT 8-1 |
| | | (td, J = 9.0, 3.9 Hz, 2H), 4.34-4.21 (m, 1H), 4.21- | in step 1 |
| | | 3.56 (m, 13H), 3.56-3.38 (m, 2H), 3.25-2.93 (m, | |
| | | 3H), 2.93-2.58 (m, 4H), 2.56-2.05 (m, 13H), 2.05- | |
| | | 1.77 (m, 5H), 1.76-1.43 (m, 3H), 0.97-0.74 (m, | |
| | | 3H). | |
| 1-10 | 1048.9 | δ 7.81-7.66 (m, 2H), 7.58 (s, 1H), 7.51 (d, J = 7.9 Hz, | INT 5-4 and |
| | | 1H), 7.37-7.32 (m, 1H), 7.28 (t, J = 9.4 Hz, 1H), 7.17- | INT 12-1 |
| | | 7.03 (m, 1H), 5.16 (dd, J = 13.3, 5.1 Hz, 1H), 4.82- | in step 1 |
| | | 4.22 (m, 9H), 4.18-3.55 (m, 8H), 3.54-3.38 (m, 4H), | |
| | | 3.25-3.02 (m, 3H), 3.01-2.72 (m, 3H), 2.61-1.90 | |
| | | (m, 18H), 1.75-1.47 (m, 4H), 0.94-0.78 (m, 3H). | |
| 1-11 | 1023.9 | δ 7.75-7.62 (m, 2H), 7.44-6.92 (m, 5H), 4.84-4.49 | INT 5-4 and |
| | | (m, 5H), 4.46-4.24 (m, 4H), 4.22-3.37 (m, 16H), | INT 7-1 |
| | | 3.27-2.99 (m, 3H), 2.95-2.63 (m, 3H), 2.60-2.07 | in step 1 |
| | | (m, 12H), 2.06-1.91 (m, 4H), 1.87-1.68 (m, 2H), | |
| | | 1.67-1.41 (m, 2H), 1.04-0.72 (m, 3H). | |
| 1-12 | 1024.6 | δ 7.75-7.55 (m, 2H), 7.40-6.93 (m, 5H), 4.84-4.18 | INT 5-4 and |
| | | (m, 9H), 4.18-3.60 (m, 12H), 3.56-3.40 (m, 2H), | INT 7-2 |
| | | 3.18-2.96 (m, 3H), 2.93-2.58 (m, 4H), 2.57-1.81 | in step 1 |
| | | (m, 17H), 1.77-1.39 (m, 4H), 0.98-0.70 (m, 3H). | |
| 1-13 | 1097.6 | δ 7.77-7.58 (m, 2H), 7.33-7.29 (m, 1H), 7.26 (t, J = | INT 5-4 and |
| | | 9.3 Hz, 1H), 7.13-7.04 (m, 3H), 5.11 (dd, J = 13.3, | INT 17-4 |
| | | 5.2 Hz, 1H), 4.79-4.63 (m, 2H), 4.64-4.46 (m, 3H), | in step 1 |
| | | 4.46-4.26 (m, 4H), 4.14-3.36 (m, 16H), 3.25-3.01 | |
| | | (m, 6H), 2.91 (ddd, J = 18.3, 13.5, 5.4 Hz, 1H), 2.83- | |
| | | 2.71 (m, 2H), 2.54-2.02 (m, 13H), 2.00-1.93 (m, | |
| | | 3H), 1.63-1.43 (m, 4H), 1.25 (s, 3H), 0.84 (dt, J = | |
| | | 19.0, 7.4 Hz, 3H). | |
| 1-14 | 1069.5 | δ 7.78-7.60 (m, 2H), 7.32-7.24 (m, 2H), 7.14-7.03 | INT 5-4 and |
| | | (m, 3H), 5.12 (dd, J = 13.3, 5.2 Hz, 1H), 4.48-4.77 | INT 17-1 |
| | | (m, 5H), 4.43-4.29 (m, 5H), 4.11-3.80 (m, 6H), | in step 1 |
| | | 3.79-3.57 (m, 4H), 3.55-3.36 (m, 4H), 3.21-3.03 (m, | |
| | | 5H), 2.74-2.95 (m, 4H), 2.48-2.02 (m, 17H), 1.74- | |
| | | 1.62 (m, 3H), 0.88-0.80 (m, 3H). | |
| 1-15 | 1083.6 | δ 7.71-7.59 (m, 2H), 7.37-7.20 (m, 2H), 7.16-6.99 | INT 5-4 and |
| | | (m, 3H), 5.10 (dd, J = 13.3, 5.1 Hz, 1H), 4.78-4.65 | INT 17-6 |
| | | (m, 2H), 4.65-4.50 (m, 2H), 4.47-4.29 (m, 4H), | in step 1 |
| | | 4.16-3.81 (m, 7H), 3.80-3.60 (m, 4H), 3.52-3.35 (m, | |
| | | 2H), 3.21-2.99 (m, 7H), 3.00-2.71 (m, 5H), 2.55- | |
| | | 2.02 (m, 16H), 2.00-1.93 (m, 2H), 1.89-1.78 (m, | |
| | | 2H), 1.50-1.18 (m, 2H), 0.85 (dt, J = 13.7, 7.4 Hz, | |
| | | 3H). | |
| 1-16 | 1082.6 | δ 7.72-7.61 (m, 2H), 7.36-7.21 (m, 2H), 7.20-6.99 | INT 5-4 and |
| | | (m, 3H), 5.11 (dd, J = 13.3, 5.1 Hz, 1H), 4.80-4.22 | INT 17-5 |
| | | (m, 8H), 4.19-3.52 (m, 11H), 3.51-3.36 (m, 1H), | in step 1 |
| | | 3.23-3.02 (m, 3H), 3.02-2.65 (m, 6H), 2.54-1.90 | |
| | | (m, 15H), 1.88-1.50 (m, 6H), 1.37-1.25 (m, 2H), | |
| | | 1.22-1.15 (m, 2H), 1.11-1.00 (m, 2H), 0.84 (dt, J = | |
| | | 23.2, 7.4 Hz, 3H). | |
| 1-17 | 1094.6 | δ 7.71-7.56 (m, 3H), 7.33-7.22 (m, 3H), 7.13 (d, | INT 5-4 and |
| | | J = 2.7 Hz, 1H), 4.79-4.22 (m, 11H), 4.16-3.81 (m, | INT 16-1 |
| | | 8H), 3.80-3.54 (m, 4H), 3.51-3.35 (m, 2H), 3.27- | in step 1 |
| | | 3.01 (m, 4H), 3.00-2.65 (m, 4H), 2.57-2.02 (m, | |
| | | 20H), 2.01-1.92 (m, 2H), 1.68-1.52 (m, 2H), | |
| | | 0.92-0.78 (m, 3H). | |
| 1-18 | 1067.6 | δ 7.91 (dd, J = 12.2, 8.9 Hz, 1H), 7.75-7.58 (m, 2H), | INT 5-4 and |
| | | 7.36-7.18 (m, 3H), 7.08 (dd, J = 36.2, 2.6 Hz, 1H), | INT 14-2 |
| | | 4.80-4.46 (m, 4H), 4.45-4.13 (m, 5H), 4.13-3.88 | in step 1 |
| | | (m, 6H), 3.88-3.55 (m, 6H), 3.55-3.36 (m, 3H), | |
| | | 3.26-3.01 (m, 2H), 2.86-2.69 (m, 4H), 2.60-1.88 (m, | |
| | | 18H), 1.84-1.64 (m, 4H), 1.61-1.50 (m, 1H), 1.45- | |
| | | 1.33 (m, 1H), 1.25-1.14 (m, 2H), 0.84 (dt, J = 20.3, | |
| | | 7.3 Hz, 3H). | |
| 1-19 | 1081.6 | δ 7.91 (d, J = 8.9 Hz, 1H), 7.75-7.63 (m, 2H), 7.38- | INT 5-4 and |
| | | 7.20 (m, 3H), 7.08 (dd, J = 37.6, 2.6 Hz, 1H), 4.79- | INT 14-3 |
| | | 4.23 (m, 8H), 4.23-3.82 (m, 10H), 3.82-3.65 (m, | in step 1 |
| | | 4H), 3.65-3.50 (m, 3H), 3.50-3.36 (m, 1H), 3.25- | |
| | | 3.00 (m, 2H), 2.90-2.67 (m, 3H), 2.58-1.92 (m, | |
| | | 18H), 1.91-1.80 (m, 1H), 1.79-1.55 (m, 4H), 1.34- | |
| | | 1.24 (m, 2H), 1.15-1.05 (m, 2H), 0.84 (dt, J = 21.0, | |
| | | 7.4 Hz, 3H). | |
| 1-20 | 1082.6 | δ 7.71-7.60 (m, 2H), 7.30 (t, J = 3.0 Hz, 1H), 7.27- | INT 5-4 and |
| | | 7.21 (m, 1H), 7.14-7.02 (m, 3H), 4.80-4.65 (m, 2H), | INT 14-1 |
| | | 4.64-4.49 (m, 2H), 4.45-4.31 (m, 3H), 4.11-3.60 | in step 1 |
| | | (m, 16H), 3.51-3.35 (m, 3H), 3.23-2.96 (m, 7H), | |
| | | 2.86-2.63 (m, 2H), 2.54-2.06 (m, 17H), 2.05-1.89 | |
| | | (m, 3H), 1.60 (d, J = 13.2 Hz, 2H), 0.85 (dt, J = 14.0, | |
| | | 7.4 Hz, 3H). | |
| 1-21 | 1069.6 | δ 7.69-7.59 (m, 2H), 7.32-7.19 (m, 2H), 7.12 (d, J = | INT 5-4 and |
| | | 2.7 Hz, 1H), 7.09-7.01 (m, 2H), 5.10 (dd, J = 13.3, | INT 17-3 |
| | | 5.1 Hz, 1H), 4.79-4.64 (m, 2H), 4.64-4.49 (m, 2H), | in step 1 |
| | | 4.44-4.32 (m, 5H), 4.14-3.82 (m, 7H), 3.81-3.56 | |
| | | (m, 4H), 3.53-3.33 (m, 5H), 3.23-3.01 (m, 3H), | |
| | | 2.97-2.72 (m, 5H), 2.51-2.02 (m, 17H), 2.02-1.92 (m, | |
| | | 1H), 1.85-1.72 (m, 2H), 0.85 (dt, J = 14.0, 7.3 Hz, | |
| | | 3H). | |
| 1-22 | 1082.1 | δ 7.68 (dd, J = 9.0, 5.8 Hz, 1H), 7.63 (d, J = 8.7 Hz, | INT 5-4 and |
| | | 1H), 7.33-7.30 (m, 1H), 7.26 (t, J = 9.4 Hz, 1H), 7.08 | INT 14-4 |
| | | (dd, J = 35.4, 2.7 Hz, 1H), 6.99 (dd, J = 9.0, 2.0 Hz, | in step 1 |
| | | 1H), 6.93 (d, J = 2.0 Hz, 1H), 4.80-4.40 (m, 5H), | |
| | | 4.37-4.27 (m, 2H), 4.26-4.12 (m, 2H), 4.12-3.81 (m, | |
| | | 9H), 3.80-3.54 (m, 5H), 3.47-3.36 (m, 1H), 3.24- | |
| | | 3.04 (m, 6H), 2.95-2.67 (m, 5H), 2.48-2.04 (m, | |
| | | 16H), 2.02-1.91 (m, 1H), 1.88-1.72 (m, 3H), 1.32- | |
| | | 1.20 (m, 2H), 0.84 (dt, J = 18.4, 7.4 Hz, 3H). | |
| 1-23 | 1068.6 | δ 7.71-7.66 (m, 2H), 7.35-7.31 (m, 1H), 7.31-7.18 | INT 5-4 and |
| | | (m, 3H), 7.12 (dd, J = 34.7, 2.7 Hz, 1H), 5.12 (dd, J = | INT 17-2 |
| | | 13.3, 5.1 Hz, 1H), 4.78-4.67 (m, 2H), 4.65-4.47 (m, | in step 1 |
| | | 3H), 4.47-4.26 (m, 4H), 4.25-3.57 (m, 12H), 3.53- | |
| | | 3.36 (m, 1H), 3.26-3.05 (m, 2H), 3.04-2.84 (m, | |
| | | 2H), 2.83-2.64 (m, 3H), 2.54-2.04 (m, 15H), 1.86- | |
| | | 1.62 (m, 4H), 1.47-1.25 (m, 4H), 1.19-1.07 (m, | |
| | | 2H), 0.87 (dt, J = 18.6, 7.4 Hz, 3H). | |
| 1-24 | 1036.5 | δ 7.85-7.76 (m, 2H), 7.59 (d, J = 8.4 Hz, 1H), 7.55- | INT 5-2 and |
| | | 7.50 (m, 1H), 7.50-7.42 (m, 2H), 7.34-7.25 (m, 3H), | INT 19-1 |
| | | 5.18 (dd, J = 13.3, 5.1 Hz, 1H), 4.82-4.64 (m, 2H), | in step 1 |
| | | 4.63-4.43 (m, 4H), 4.40-4.25 (m, 2H), 4.23-4.13 | |
| | | (m, 2H), 4.12-3.83 (m, 5H), 3.79-3.56 (m, 4H), | |
| | | 3.26-2.96 (m, 7H), 2.96-2.72 (m, 4H), 2.58-1.98 (m, | |
| | | 19H), 1.92-1.58 (m, 2H), 1.42-1.12 (m, 3H). | |
| 1-25 | 1082.6 | δ 7.81 (d, J = 7.9 Hz, 1H), 7.71 (dd, J = 9.0, 5.8 Hz, | INT 5-4 and |
| | | 1H), 7.58-7.51 (m, 1H), 7.47 (d, J = 7.8 Hz, 1H), 7.34 | INT 19-1 |
| | | (t, J = 2.6 Hz, 1H), 7.28 (t, J = 9.4 Hz, 1H), 7.10 (dd, | in step 1 |
| | | J = 35.7, 2.7 Hz, 1H), 5.18 (dd, J = 13.3, 5.1 Hz, 1H), | |
| | | 4.82-4.71 (m, 1H), 4.70-4.42 (m, 5H), 4.42-4.15 | |
| | | (m, 4H), 4.15-3.82 (m, 5H), 3.83-3.66 (m, 3H), 3.65- | |
| | | 3.56 (m, 1H), 3.23-3.02 (m, 7H), 3.00-2.75 (m, | |
| | | 4H), 2.58-2.03 (m, 20H), 1.92-1.72 (m, 2H), 1.42- | |
| | | 1.21 (m, 4H), 0.97-0.77 (m, 3H). | |
| 1-26 | 1022.5 | δ 7.77 (d, J = 8.3 Hz, 1H), 7.66 (d, J = 8.6 Hz, 1H), | INT 5-2 and |
| | | 7.59 (d, J = 8.5 Hz, 1H), 7.49-7.41 (m, 1H), 7.35- | INT 17-2 |
| | | 7.25 (m, 3H), 7.18-7.04 (m, 2H), 5.16-5.08 (m, 1H), | in step 1 |
| | | 4.80-4.65 (m, 2H), 4.63-4.20 (m, 6H), 4.19-3.78 | |
| | | (m, 10H), 3.77-3.38 (m, 4H), 3.24-3.10 (m, 2H), | |
| | | 2.98-2.74 (m, 4H), 2.74-2.55 (m, 2H), 2.54-2.04 | |
| | | (m, 14H), 2.03-1.96 (m, 1H), 1.87-1.36 (m, 5H), | |
| | | 1.20-1.01 (m, 2H). | |
| 1-27 | 1084.6 | δ 7.75-7.61 (m, 2H), 7.39-7.22 (m, 2H), 7.21-7.03 | INT 5-4 and |
| | | (m, 3H), 5.12 (dd, J = 13.3, 5.1 Hz, 1H), 4.81-4.49 | INT 9-1 |
| | | (m, 4H), 4.47-4.25 (m, 4H), 4.15-3.55 (m, 14H), | in step 1 |
| | | 3.29-3.03 (m, 6H), 2.97-2.85 (m, 1H), 2.83-2.74 | |
| | | (m, 1H), 2.54-2.05 (m, 16H), 2.03-1.96 (m, 2H), | |
| | | 1.91-1.74 (m, 2H), 1.74-1.60 (m, 2H), 1.59-1.43 | |
| | | (m, 2H), 0.86 (m, 3H). | |
| 1-28 | 1038.5 | δ 7.79 (d, J = 8.3 Hz, 1H), 7.65 (d, J = 9.2 Hz, 1H), | INT 5-2 and |
| | | 7.58 (d, J = 8.5 Hz, 1H), 7.46 (t, J = 7.5 Hz, 1H), | INT 9-1 |
| | | 7.36-7.25 (m, 3H), 7.17-7.07 (m, 2H), 5.11 (dd, J = | in step 1 |
| | | 13.3, 5.1 Hz, 1H), 4.80-4.67 (m, 2H), 4.63-4.48 (m, 2H), | |
| | | 4.47-4.33 (m, 3H), 4.33-4.21 (m, 1H), 4.14-3.59 | |
| | | (m, 14H), 3.48-3.41 (m, 1H), 3.24-3.12 (m, 5H), | |
| | | 2.97-2.85 (m, 1H), 2.84-2.74 (m, 1H), 2.53-2.32 | |
| | | (m, 5H), 2.32-2.22 (m, 2H), 2.22-2.05 (m, 6H), 2.03- | |
| | | 1.92 (m, 3H), 1.88-1.71 (m, 2H), 1.71-1.58 (m, | |
| | | 2H), 1.57-1.38 (m, 2H). | |
| 1-29 | 1083.6 | δ 7.76-7.66 (m, 2H), 7.35-7.31 (m, 1H), 7.28 (t, | INT 5-4 and |
| | | J = 9.4 Hz, 1H), 7.23-7.16 (m, 2H), 7.15-7.03 (m, | INT 17-9 |
| | | 1H), 5.14 (dd, J = 13.3, 5.1 Hz, 1H), 4.82-4.71 (m, | in step 1 |
| | | 1H), 4.71-4.52 (m, 3H), 4.51-4.42 (m, 3H), 4.42- | |
| | | 4.28 (m, 2H), 4.28-4.14 (m, 2H), 4.14-3.83 (m, 6H), | |
| | | 3.82-3.65 (m, 3H), 3.66-3.57 (m, 2H), 3.48-3.38 | |
| | | (m, 2H), 3.22-3.03 (m, 5H), 3.00-2.74 (m, 4H), 2.56- | |
| | | 1.96 (m, 18H), 1.92-1.72 (m, 2H), 1.37-1.20 (m, | |
| | | 2H), 0.92-0.79 (m, 3H). | |
| 1-30 | 1079.4 | δ 7.90 (dd, J = 9.2, 5.6 Hz, 1H), 7.78-7.68 (m, 1H), | INT 17-9 |
| | | 7.44-7.31 (m, 2H), 7.31-7.22 (m, 1H), 7.22-7.14 | in step 1 |
| | | (m, 2H), 5.14 (dd, J = 13.3, 5.1 Hz, 1H), 4.83-3.38 | |
| | | (m, 23H), 3.28-2.70 (m, 10H), 2.64-1.67 (m, 17H), | |
| | | 1.28 (dd, J = 14.5, 7.2 Hz, 4H). | |
| 1-31 | 1063.4 | δ 8.00-7.77 (m, 1H), 7.72 (d, J = 8.4 Hz, 1H), 7.45- | INT 21-1 |
| | | 7.21 (m, 4H), 7.18-6.97 (m, 1H), 4.83-4.21 (m, | in step 1 |
| | | 10H), 4.19-3.36 (m, 13H), 3.30-2.62 (m, 10H), 2.56- | |
| | | 1.91 (m, 18H), 1.75 (s, 4H). | |
| 1-32 | 1054.5 | δ 7.71-7.63 (m, 1H), 7.33-7.28 (m, 1H), 7.28-7.20 | INT 5-4 and |
| | | (m, 1H), 7.18-7.00 (m, 4H), 5.33 (dd, J = 12.3, 5.4 | INT 28-1 |
| | | Hz, 1H), 4.78-4.70 (m, 1H), 4.65-4.45 (m, 5H), | in step 1 |
| | | 4.41-4.25 (m, 2H), 4.11-3.55 (m, 13H), 3.46-3.37 (m, | |
| | | 1H), 3.26-2.74 (m, 7H), 2.48-2.03 (m, 14H), 2.01- | |
| | | 1.84 (m, 3H), 1.65-1.52 (m, 2H), 0.89-0.77 (m, | |
| | | 3H). | |
| 1-33 | 1082.6 | δ 7.79 (dd, J = 9.1, 2.1 Hz, 1H), 7.67 (dd, J = 9.1, 5.8 | INT 5-4 and |
| | | Hz, 1H), 7.32 (t, J = 3.6 Hz, 1H), 7.25 (t, J = 9.4 Hz, | INT 37-1 |
| | | 1H), 7.11 (dd, J = 34.3, 2.6 Hz, 1H), 6.70 (dd, J = 9.2, | in step 1 |
| | | 1.9 Hz, 1H), 4.80-4.65 (m, 2H), 4.65-4.41 (m, 4H), | |
| | | 4.30 (s, 2H), 4.19 (ddd, J = 9.4, 5.1, 1.3 Hz, 1H), | |
| | | 4.16-3.91 (m, 6H), 3.83-3.65 (m, 2H), 3.59 (d, J = 6.8 | |
| | | Hz, 1H), 3.37 (dd, J = 12.2, 5.3 Hz, 1H), 3.23-3.03 | |
| | | (m, 2H), 2.95-2.60 (m, 7H), 2.54-1.89 (m, 17H), | |
| | | 1.84-1.46 (m, 7H), 1.25-0.94 (m, 6H), 0.85 (dt, J = | |
| | | 18.9, 7.4 Hz, 3H). | |
| 1-34 | 1054.6 | δ 7.80 (d, J = 9.0 Hz, 1H), 6.72 (d, J = 9.2 Hz, 1H), | INT 5-10 and |
| | | 6.69 (s, 1H), 6.64 (d, J = 2.6 Hz, 1H), 4.73 (d, J = 2.0 | INT 37-1 |
| | | Hz, 2H), 4.57 (dd, J = 14.4, 6.1 Hz, 2H), 4.50 (d, J = | in step 1 |
| | | 13.4 Hz, 2H), 4.30 (s, 2H), 4.20 (dd, J = 9.4, 5.1 Hz, | |
| | | 1H), 4.16-4.01 (m, 4H), 4.01-3.89 (m, 2H), 3.82 | |
| | | (ddd, J = 13.0, 8.6, 4.3 Hz, 1H), 3.69 (td, J = 11.7, 2.8 | |
| | | Hz, 1H), 3.61 (dd, J = 11.7, 5.1 Hz, 1H), 3.44-3.35 | |
| | | (m, 1H), 3.27-3.12 (m, 2H), 3.05 (s, 1H), 2.98-2.85 | |
| | | (m, 2H), 2.85-2.63 (m, 8H), 2.51-2.02 (m, 14H), | |
| | | 2.02-1.82 (m, 3H), 1.82-1.51 (m, 8H), 1.17 (d, J = | |
| | | 10.5 Hz, 4H), 1.11-0.97 (m, 2H), 0.84 (d, J = 7.0 Hz, | |
| | | 3H). | |
| 1-35 | 1077.6 | δ 7.98-7.82 (m, 2H), 7.70 (s, 1H), 7.47-7.15 (m, | INT 14-3 |
| | | 4H), 4.71 (q, J = 12.2 Hz, 2H), 4.57 (dd, J = 14.4, 7.0 | in step 1 |
| | | Hz, 2H), 4.41 (dd, J = 9.6, 5.1 Hz, 2H), 4.29 (s, 2H), | |
| | | 4.22-3.91 (m, 8H), 3.91-3.66 (m, 5H), 3.57 (t, J = | |
| | | 11.9 Hz, 3H), 3.44 (s, 1H), 3.26-3.03 (m, 2H), 2.90- | |
| | | 2.68 (m, 4H), 2.60-1.92 (m, 16H), 1.92-1.49 (m, | |
| | | 6H), 1.36-1.22 (m, 2H), 1.10 (dd, J = 23.8, 11.0 Hz, | |
| | | 2H). | |
| 1-36 | 1117.6 | δ 7.83 (d, J = 8.9 Hz, 1H), 7.68 (dd, J = 9.1, 5.8 Hz, | INT 5-4 and |
| | | 1H), 7.54-7.48 (m, 1H), 7.34-7.19 (m, 3H), 7.10 | INT 14-8 |
| | | (dd, J = 37.5, 2.6 Hz, 1H), 4.84-4.65 (m, 2H), 4.65- | in step 1 |
| | | 4.46 (m, 2H), 4.46-4.16 (m, 5H), 4.15-3.56 (m, | |
| | | 12H), 3.42 (t, J = 11.9 Hz, 3H), 3.22-3.04 (m, 2H), | |
| | | 2.91-2.67 (m, 4H), 2.55-2.04 (m, 19H), 2.03-1.69 | |
| | | (m, 5H), 1.45 (q, J = 10.8, 8.7 Hz, 2H), 0.85 (dt, J = | |
| | | 19.4, 7.4 Hz, 3H). | |
| 1-37 | 1118.6 | δ 7.82 (dd, J = 9.0, 1.1 Hz, 1H), 7.68 (dd, J = 9.1, 5.8 | INT 5-4 and |
| GS- | | Hz, 1H), 7.36-7.18 (m, 2H), 7.09 (dd, J = 36.0, 2.6 | INT 37-5 |
| 1666 | | Hz, 1H), 6.74 (d, J = 9.3 Hz, 1H), 4.81-4.45 (m, 6H), | in step 1 |
| 274 | | 4.45-4.14 (m, 5H), 4.14-3.82 (m, 7H), 3.82-3.55 | |
| | | (m, 3H), 3.43 (s, 1H), 3.23-3.23 (m, 2H), 2.99-2.63 | |
| | | (m, 6H), 2.53-2.02 (m, 17H), 2.02-1.67 (m, 5H), | |
| | | 1.58-1.34 (m, 4H), 0.84 (dt, J = 20.9, 7.4 Hz, 3H). | |
| 1-38 | 1117.6 | δ 7.76 (dd, J = 8.9, 1.8 Hz, 1H), 7.67 (dd, J = 9.1, 5.8 | INT 5-4 and |
| GS- | | Hz, 1H), 7.46-7.16 (m, 4H), 7.10 (dd, J = 35.1, 2.6 | INT 14-11 |
| 1666 | | Hz, 1H), 4.81-4.64 (m, 2H), 4.62-4.46 (m, 2H), 4.36 | in step 1 |
| 277 | | (ddd, J = 9.4, 5.2, 1.3 Hz, 3H), 4.17-3.82 (m, 9H), | |
| | | 3.82-3.35 (m, 8H), 3.27-3.01 (m, 4H), 2.88-2.65 | |
| | | (m, 2H), 2.56-1.88 (m, 24H), 1.74 (dt, J = 31.9, 12.9 | |
| | | Hz, 2H), 0.85 (dt, J = 19.3, 7.3 Hz, 3H). | |
| 1-39 | 1118.6 | δ 7.82 (dd, J = 9.1, 2.9 Hz, 1H), 7.67 (dd, J = 9.1, 5.8 | INT 5-4 and |
| | | Hz, 1H), 7.36-7.16 (m, 2H), 7.09 (dd, J = 32.4, 2.6 | INT 37-6 |
| | | Hz, 1H), 6.75 (dd, J = 9.1, 2.4 Hz, 1H), 4.82-4.46 (m, | in step 1 |
| | | 4H), 4.45-4.15 (m, 5H), 4.15-3.83 (m, 9H), 3.83- | |
| | | 3.54 (m, 3H), 3.42 (s, 2H), 3.25-3.02 (m, 5H), 2.85- | |
| | | 2.63 (m, 2H), 2.55-1.52 (m, 26H), 0.85 (dt, J = 21.0, | |
| | | 7.4 Hz, 3H). | |
| 1-40 | 1072.3 | δ 8.62 (s, 1H), 6.68 (d, J = 2.7 Hz, 1H), 6.63 (d, J = 2.7 | INT 5-10 and |
| | | Hz, 1H), 4.77 (d, J = 12.2 Hz, 1H), 4.66 (d, J = 12.2 | INT 31-4 |
| | | Hz, 1H), 4.62-4.52 (m, 2H), 4.50-4.36 (m, 1H), 4.34- | in step 1 |
| | | 4.29 (m, 1H), 4.26-4.18 (m, 1H), 4.17-4.13 (m, | |
| | | 3H), 4.09-3.85 (m, 6H), 3.85-3.56 (m, 5H), 3.50- | |
| | | 3.37 (m, 2H), 3.24-3.01 (m, 5H), 2.93-2.72 (m, 6H), | |
| | | 2.61-2.04 (m, 14H), 1.99-1.68 (m, 9H), 1.45-1.31 | |
| | | (m, 2H), 1.27-1.23 (m, 2H), 1.18-1.04 (m, 2H), 0.84 | |
| | | (d, J = 7.0 Hz, 3H). | |
| 1-41 | 1170.6 | δ 7.90 (d, J = 8.8 Hz, 1H), 7.70-7.64 (m, 2H), 7.36- | INT 5-4 and |
| | | 7.29 (m, 2H), 7.25 (t, J = 9.4 Hz, 1H), 7.18-7.01 (m, | INT 24-1 |
| | | 4H), 4.75 (t, J = 11.4 Hz, 1H), 4.69-4.47 (m, 3H), | in step 1 |
| | | 4.44-4.25 (m, 3H), 4.08 (s, 3H), 4.04-3.82 (m, 6H), | |
| | | 3.80-3.38 (m, 10H), 3.21-3.02 (m, 3H), 3.02-2.90 | |
| | | (m, 1H), 2.88-2.70 (m, 6H), 2.55-2.22 (m, 10H), | |
| | | 2.22-2.04 (m, 9H), 1.57 (s, 4H), 0.91-0.76 (m, 3H). | |
| 1-42 | 1077.5 | δ 7.96-7.82 (m, 2H), 7.70-7.57 (m, 1H), 7.43-7.16 | INT 5-13 and |
| | | (m, 4H), 4.84-4.08 (m, 12H), 4.08-3.38 (m, 12H), | INT 14-3 in |
| | | 3.28-2.57 (m, 8H), 2.55-1.89 (m, 18H), 1.89-0.99 | step 1 |
| | | (m, 7H). | |
| 1-43 | 1080.6 | δ 7.79-7.62 (m, 3H), 7.35 (d, J = 1.3 Hz, 1H), 7.33- | INT 5-4 and |
| | | 7.20 (m, 3H), 7.08 (dd, J = 37.6, 2.6 Hz, 1H), 4.80- | INT 38-4 |
| | | 4.24 (m, 7H), 4.23-3.88 (m, 11H), 3.80-3.65 (m, | in step 1 |
| | | 5H), 3.63-3.55 (m, 1H), 3.48-3.36 (m, 1H), 3.24- | |
| | | 2.99 (m, 2H), 2.92-2.64 (m, 3H), 2.54-1.85 (m, | |
| | | 19H), 1.82-1.52 (m, 5H), 1.31 (t, J = 7.4 Hz, 2H), | |
| | | 1.11 (d, J = 12.8 Hz, 2H), 0.84 (dt, J = 21.0, 7.4 Hz, | |
| | | 3H). | |
| 1-44 | 1107.6 | δ 7.91 (d, J = 8.8 Hz, 1H), 7.85 (d, J = 2.3 Hz, 1H), | INT 5-4 and |
| | | 7.68 (dd, J = 9.1, 5.7 Hz, 1H), 7.41-7.19 (m, 3H), | INT 38-5 |
| | | 7.08 (dd, J = 37.0, 2.6 Hz, 1H), 4.81-4.24 (m, 7H), | in step 1 |
| | | 4.24-3.82 (m, 7H), 3.82-3.52 (m, 7H), 3.43 (s, 1H), | |
| | | 3.12 (ddt, J = 29.0, 13.9, 6.2 Hz, 2H), 2.92-2.69 (m, | |
| | | 4H), 2.59-1.82 (m, 19H), 1.82-1.52 (m, 5H), 1.29 | |
| | | (t, J = 6.9 Hz, 2H), 1.24-1.16 (m, 4H), 1.16-1.04 | |
| | | (m, 2H), 0.85 (dt, J = 20.4, 7.3 Hz, 3H). | |
| 1-45 | 1117.6 | δ 7.68 (dd, J = 9.1, 5.8 Hz, 1H), 7.37-7.17 (m, 3H), | INT 5-4 and |
| | | 7.09 (dd, J = 36.0, 2.6 Hz, 1H), 4.81-4.22 (m, 7H), | INT 46-1 |
| | | 4.22-3.83 (m, 9H), 3.83-3.65 (m, 2H), 3.60 (s, 1H), | in step 1 |
| | | 3.50-3.36 (m, 1H), 3.28-3.03 (m, 6H), 2.91-2.63 | |
| | | (m, 4H), 2.56-1.89 (m, 16H), 1.83-1.44 (m, 6H), | |
| | | 1.34 (ddd, J = 21.3, 10.5, 5.9 Hz, 2H), 1.21 (t, J = 6.8 | |
| | | Hz, 2H), 1.15-0.97 (m, 2H), 0.85 (dt, J = 20.8, 7.4 | |
| | | Hz, 3H). | |
| 1-46 | 1096.5 | δ 7.70 (dd, J = 9.1, 1.7 Hz, 1H), 7.58 (dd, J = 9.1, 5.9 | INT 5-15 and |
| | | Hz, 1H), 7.32-7.06 (m, 1H), 6.98 (d, J = 10.7 Hz, | INT 37-1 |
| | | 1H), 6.62 (d, J = 9.9 Hz, 1H), 4.70-4.48 (m, 3H), 4.48- | in step 1 |
| | | 3.84 (m, 11H), 3.78 (d, J = 1.8 Hz, 3H), 3.72-3.27 | |
| | | (m, 4H), 3.15-2.93 (m, 3H), 2.93-2.55 (m, 6H), | |
| | | 2.55-1.87 (m, 15H), 1.85-1.32 (m, 9H), 1.16-0.89 (m, | |
| | | 7H), 0.74 (dt, J = 15.9, 7.4 Hz, 3H). | |
| 1-47 | 1069.2 | δ 7.91 (d, J = 8.8 Hz, 1H), 7.79-7.68 (m, 2H), 7.41- | INT 5-3 and |
| | | 7.29 (m, 4H), 7.27-7.15 (m, 1H), 4.79-4.38 (m, 6H), | INT 14-3 in |
| | | 4.34-4.22 (m, 1H), 4.20-3.83 (m, 11H), 3.81-3.52 | step 1 |
| | | (m, 8H), 3.49-3.39 (m, 1H), 3.24-3.01 (m, 2H), | |
| | | 2.87-2.68 (m, 4H), 2.57-2.46 (m, 1H), 2.44-2.21 (m, | |
| | | 7H), 2.19-2.01 (m, 7H), 1.71-1.56 (m, 4H), 1.37- | |
| | | 1.25 (m, 2H), 1.17-1.04 (m, 2H). | |
| 1-48 | 1087.2 | δ 7.93 (d, J = 8.8 Hz, 1H), 7.78 (d, J = 2.0 Hz, 1H), | INT 5-16 and |
| | | 7.34 (dd, J = 8.8, 2.0 Hz, 1H), 7.15 (d, J = 2.5 Hz, 1H), | INT 14-3 in |
| | | 6.79 (d, J = 38.8 Hz, 1H), 4.77-4.61 (m, 2H), 4.53 | step 1 |
| | | (dd, J = 14.3, 6.4 Hz, 2H), 4.42 (dd, J = 9.6, 5.1 Hz, | |
| | | 1H), 4.36-4.25 (m, 1H), 4.22-3.56 (m, 17H), 3.47- | |
| | | 3.38 (m, 1H), 3.18-3.04 (m, 2H), 2.90-2.70 (m, | |
| | | 4H), 2.58-2.02 (m, 15H), 2.00-1.61 (m, 7H), 1.32 | |
| | | (t, J = 7.0 Hz, 2H), 1.13 (qd, J = 12.3, 4.2 Hz, 2H). | |
| 1-49 | 1086 | δ 7.82 (d, J = 9.0 Hz, 1H), 7.47 (s, 1H), 7.22 (d, J = 8.8 | INT 5-17 and |
| | | Hz, 1H), 6.91 (s, 1H), 6.52 (d, J = 44.2 Hz, 1H), | INT 14-3 in |
| | | 4.78-4.57 (m, 3H), 4.57-4.26 (m, 5H), 4.21-4.10 (m, | step 1 |
| | | 2H), 4.09-3.85 (m, 6H), 3.83-3.63 (m, 4H), 3.61- | |
| | | 3.53 (m, 1H), 3.48 (p, J = 1.6 Hz, 2H), 3.15-3.04 (m, | |
| | | 3H), 2.91-2.69 (m, 4H), 2.54-2.22 (m, 7H), 2.18- | |
| | | 1.89 (m, 7H), 1.83-1.50 (m, 6H), 1.36-1.27 (m, 3H), | |
| | | 1.22-1.05 (m, 3H). | |

### Examples 2-1-2-82

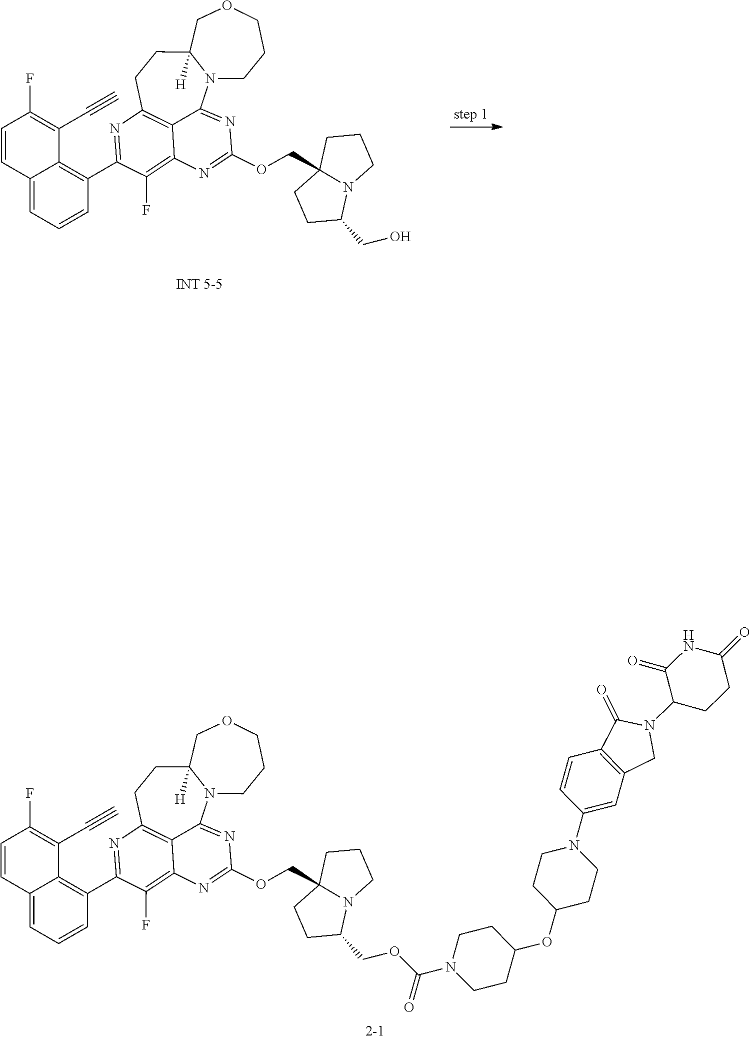

Step 1: ((3S,7aS)-7a-((((S)-2-(8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(2-(2,6-dioxopiperidin-3-yl)-1-oxoisoindolin-5-yl)piperidin-4-yl)oxy)piperidine-1-carboxylate (Example 2-1). To a solution of INT 5-5 (0.02 mmol) in tetrahydrofuran (1 mL) at 0° C. was added 4-nitrophenyl chloroformate (0.03 mmol) and diisopropylethylamine (0.1 mmol). The resulting mixture was stirred at 0° C. for 1 hour under an argon atmosphere. To the reaction mixture was added a solution of 3-(1-oxo-5-(4-(piperidin-4-yloxy)piperidin-1-yl)isoindolin-2-yl)piperidine-2,6-dione (INT 9-1) (0.06 mmol) and diisopropylethylamine (0.2 mmol) in dimethyl sulfoxide (1.0 mL). The reaction mixture was warmed to room temperature, stirred for 1 hour, then diluted with diethyl ether (10 mL) and the resulting precipitate was purified by RP-HPLC (0 to 90%, 0.1% trifluoroacetic acid in acetonitrile/0.1% trifluoroacetic acid in water). Fractions containing the product were pooled and lyophilized to yield the title compound. ¹H NMR (400 MHz, Methanol-d₄) δ 8.14 (dd, J=8.9, 6.0 Hz, 2H), 7.76-7.59 (m, 3H), 7.47 (t, J=8.9 Hz, 1H), 7.10 (d, J=7.7 Hz, 2H), 5.10 (dd, J=13.3, 5.1 Hz, 1H), 4.78-4.61 (m, 3H), 4.60-4.42 (m, 3H), 4.41-4.32 (m, 3H), 4.27 (s, 1H), 4.16-3.83 (m, 5H), 3.80-3.63 (m, 6H), 3.58 (d, J=9.4 Hz, 1H), 3.51-3.37 (m, 2H), 3.21-3.10 (m, 4H), 2.90 (ddd, J=18.3, 13.4, 5.3 Hz, 1H), 2.81-2.70 (m, 1H), 2.52-2.30 (m, 4H), 2.24 (s, 3H), 2.21-2.08 (m, 4H), 2.03 (s, 3H), 1.94 (s, 3H), 1.81 (s, 2H), 1.70-1.44 (m, 4H). LCMS: 1064.6.

The following Examples were made in a similar fashion to Example 2-1 and are shown below in Table 3B1. To prepare the below Examples, different reagents/starting materials were used than some of those described in the steps toward Example 2-1 and are noted in the last column of Table 3B2—“Changes to Procedure: Different Reagents/Starting Materials”. A person of ordinary skill in the art will readily recognize which reagents/starting materials used for the synthesis of Example 2-1 were replaced with the different reagents/starting materials noted below.

TABLE 3B1
Compounds

| Example | Structure |
|---|---|
| 2-2 | 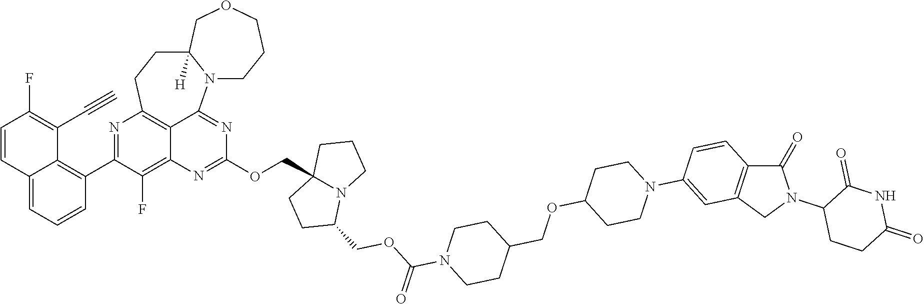 |
| 2-3 | 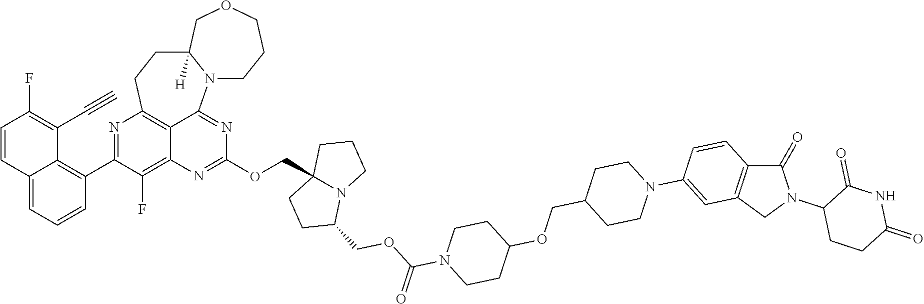 |
| 2-4 | 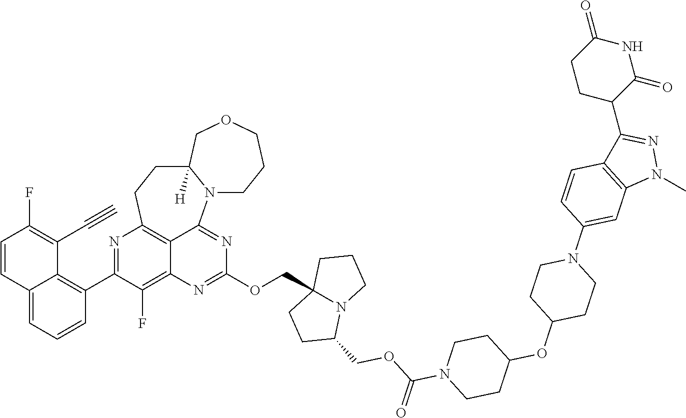 |
| 2-5 | 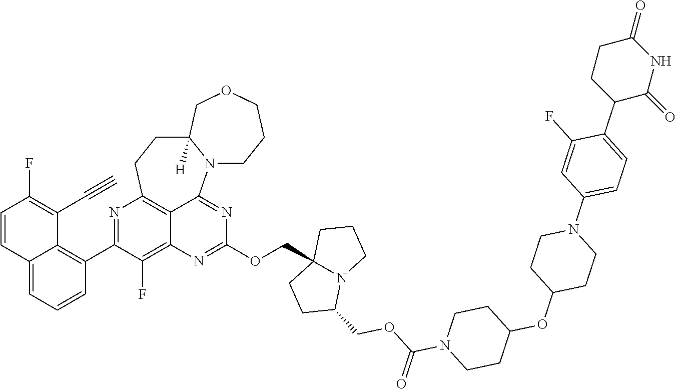 |
| 2-6 | 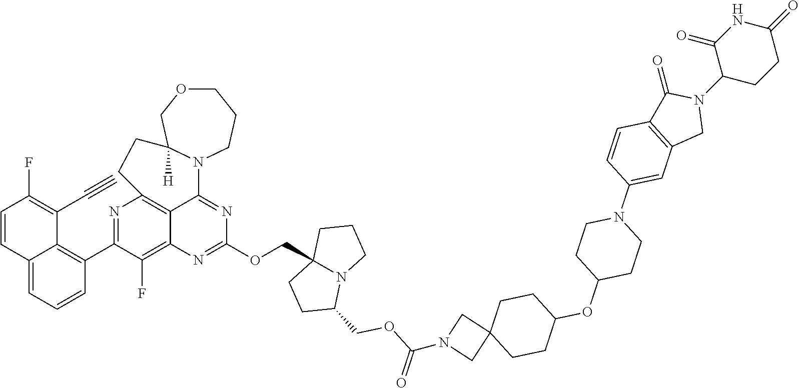 |
| 2-7 | 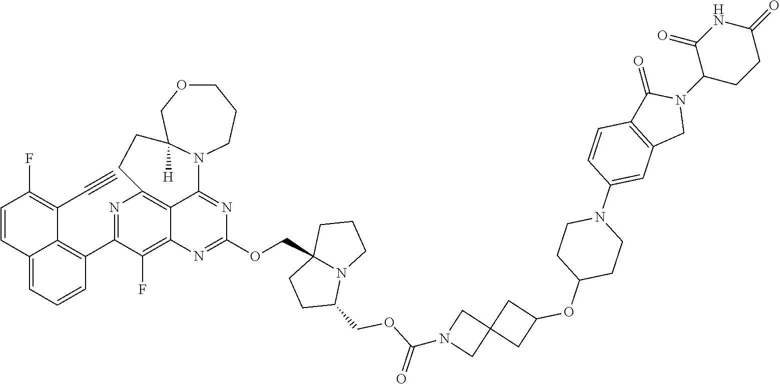 |
| 2-8 | 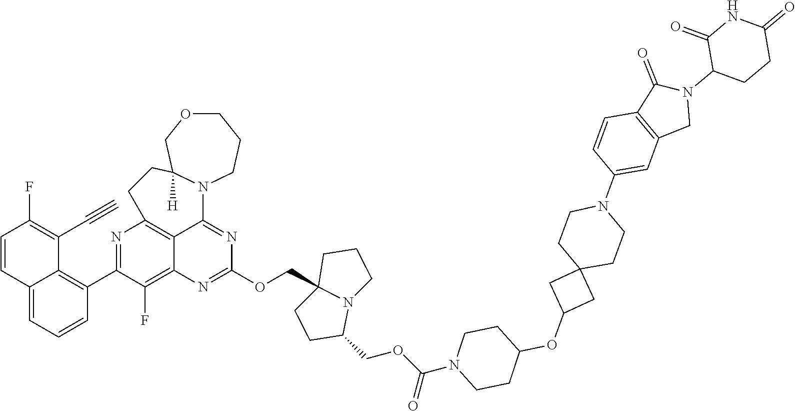 |
| 2-9 | 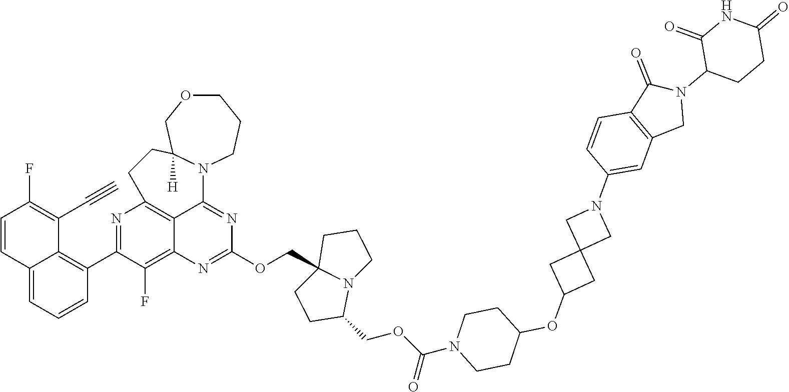 |
| 2-10 | 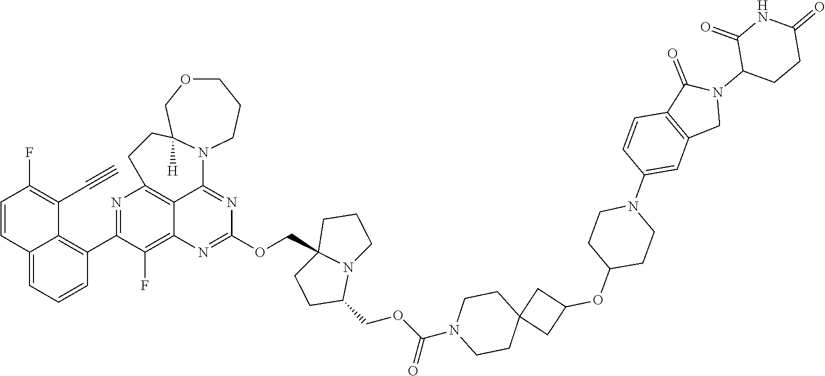 |
| 2-11 | 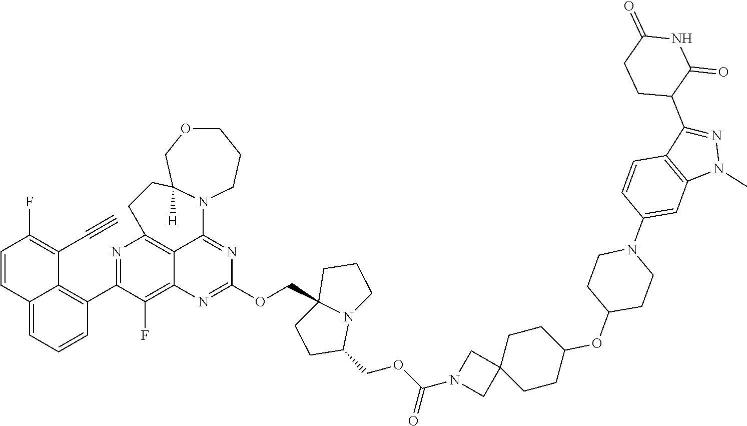 |
| 2-12 | 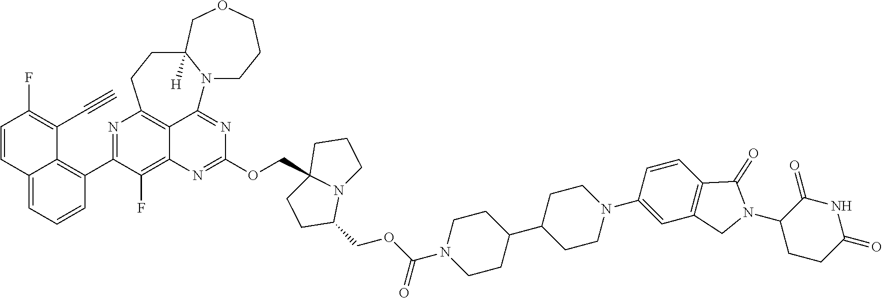 |
| 2-13 | 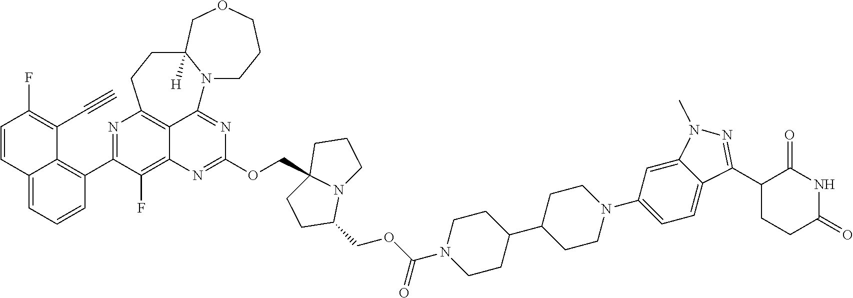 |
| 2-14 | 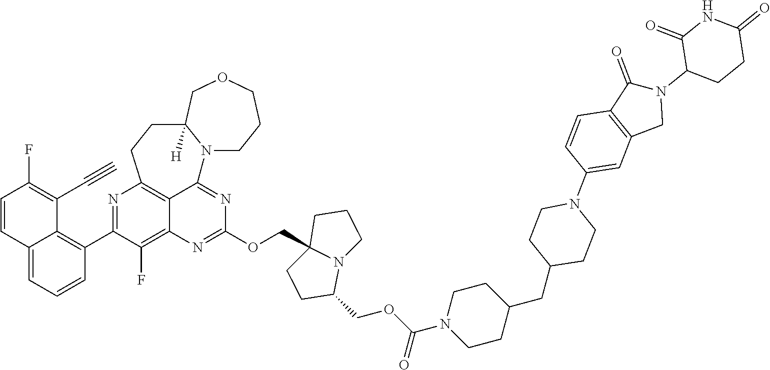 |
| 2-15 | 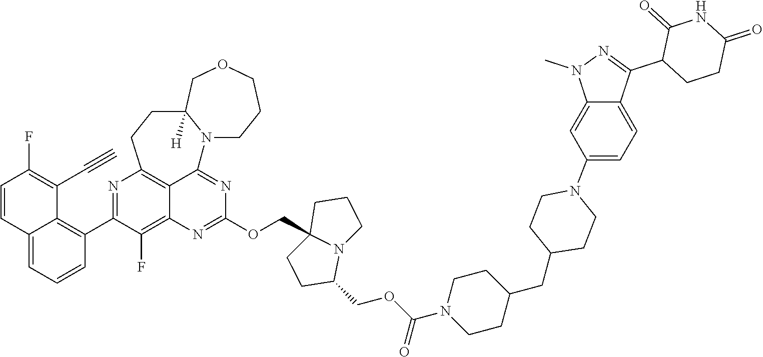 |
| 2-16 | 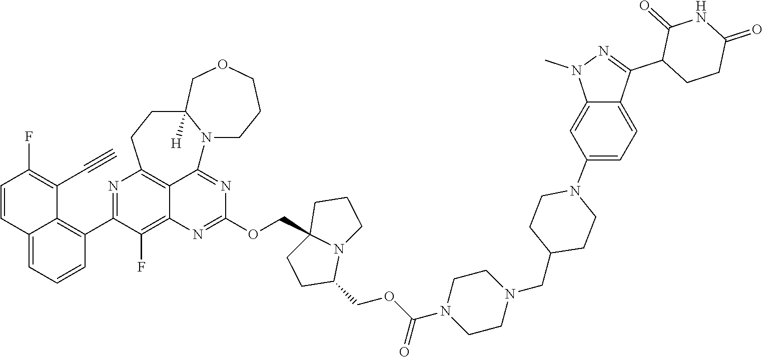 |
| 2-17 | 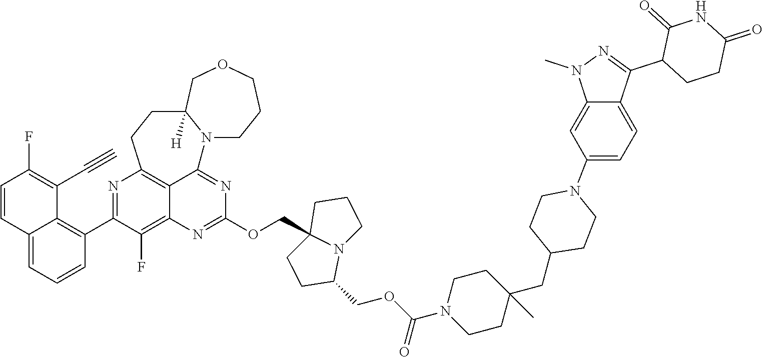 |
| 2-18 | 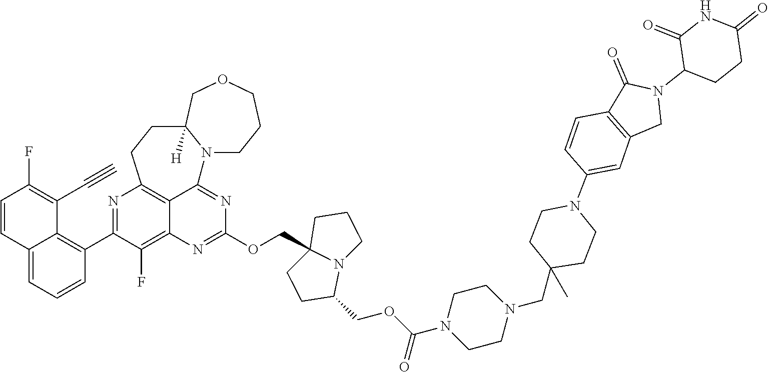 |
| 2-19 | 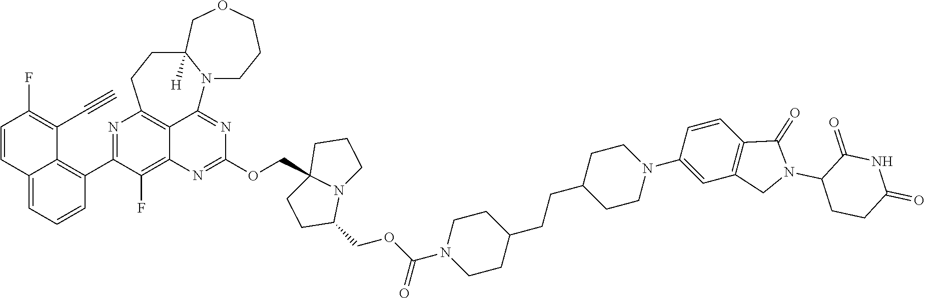 |
| 2-20 | 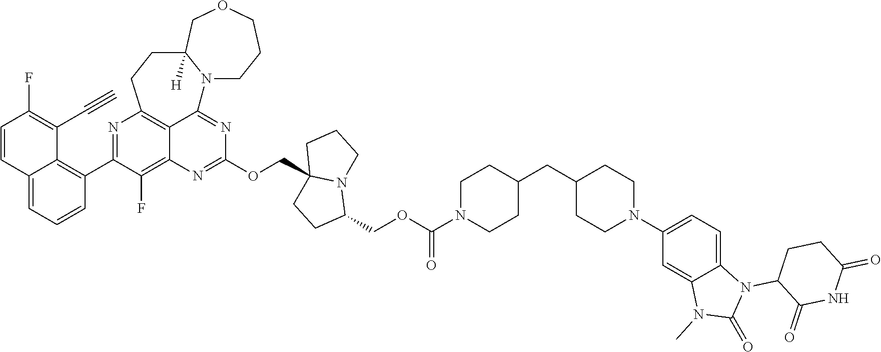 |
| 2-21 | 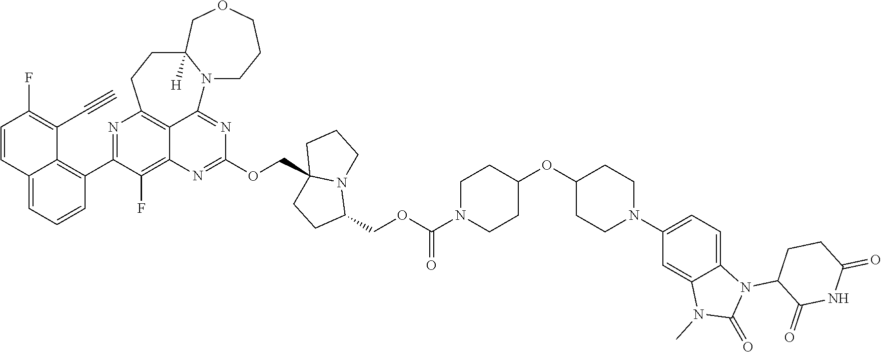 |
| 2-22 | 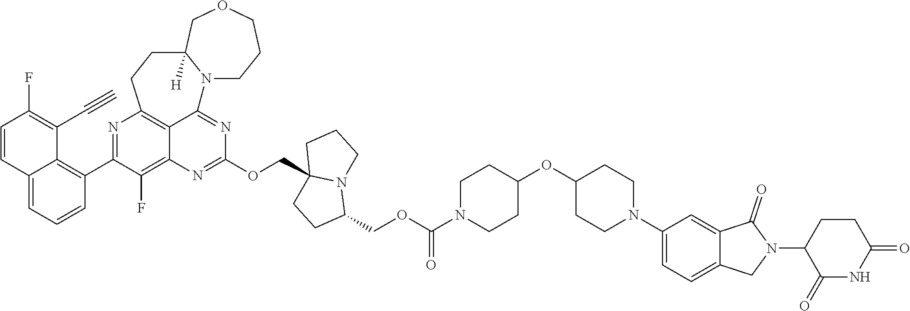 |
| 2-23 | 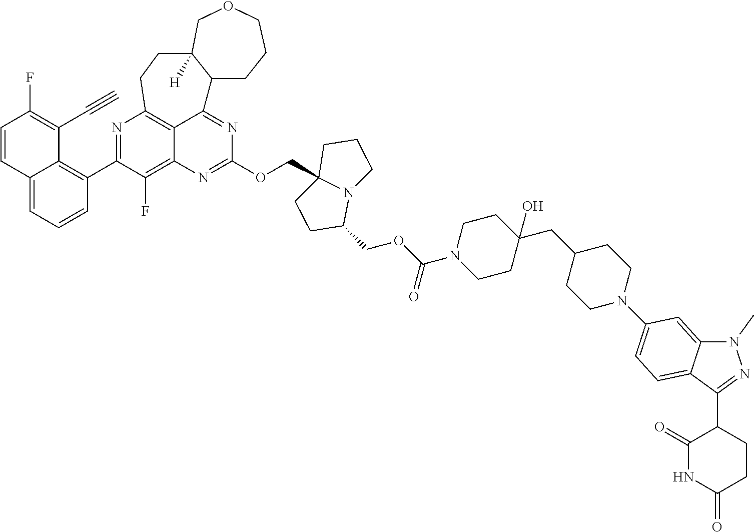 |
| 2-24 | 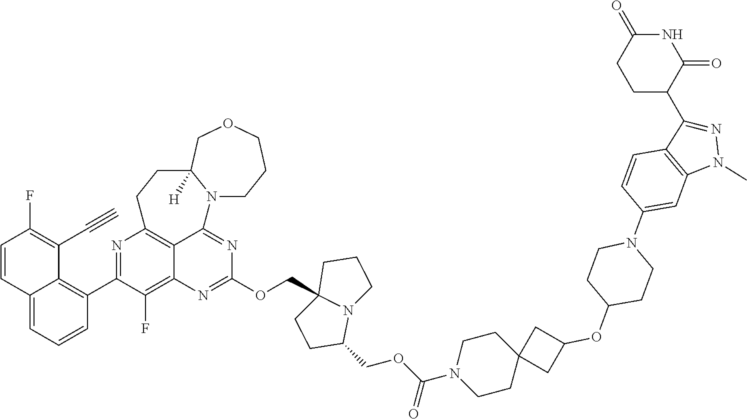 |
| 2-25 | 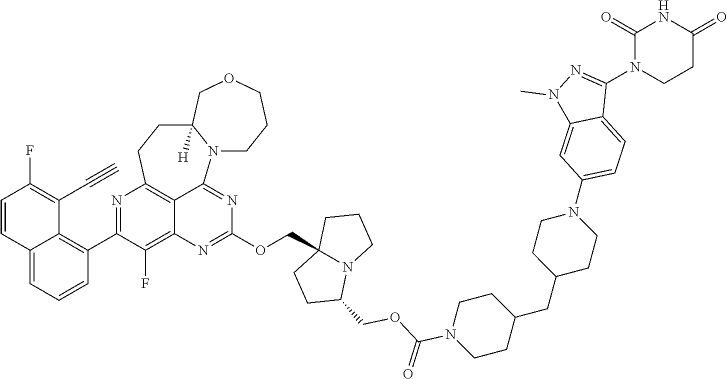 |
| 2-26 | 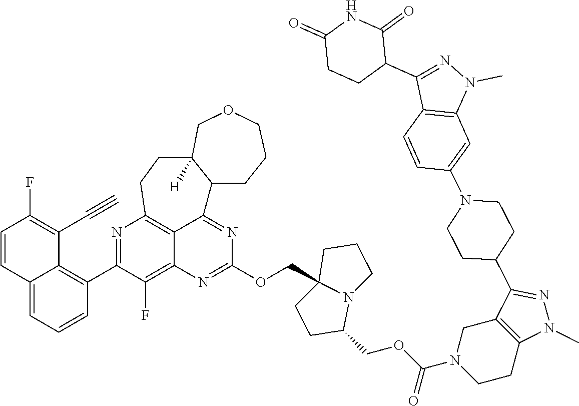 |
| 2-27 | 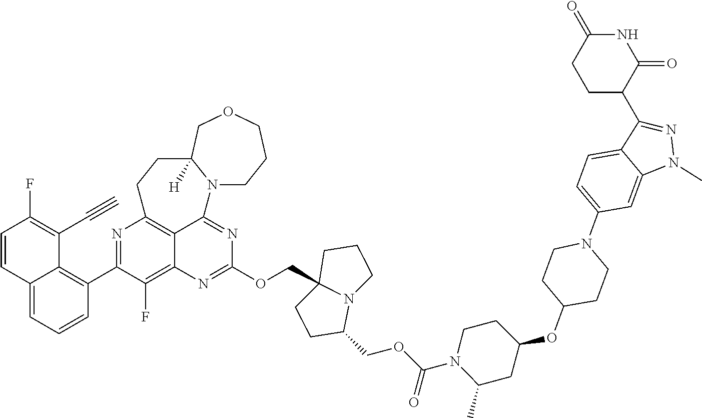 |
| 2-28 | 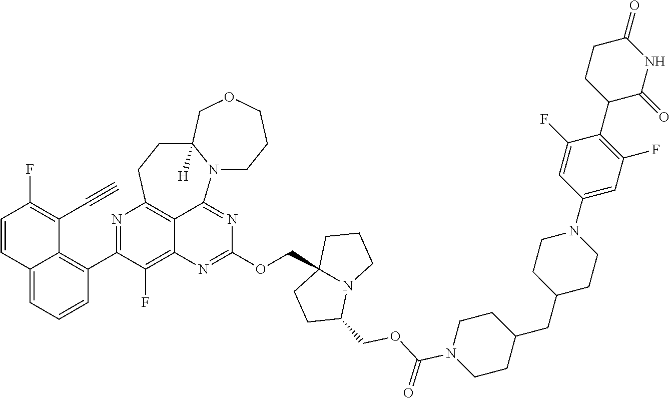 |
| 2-29 | 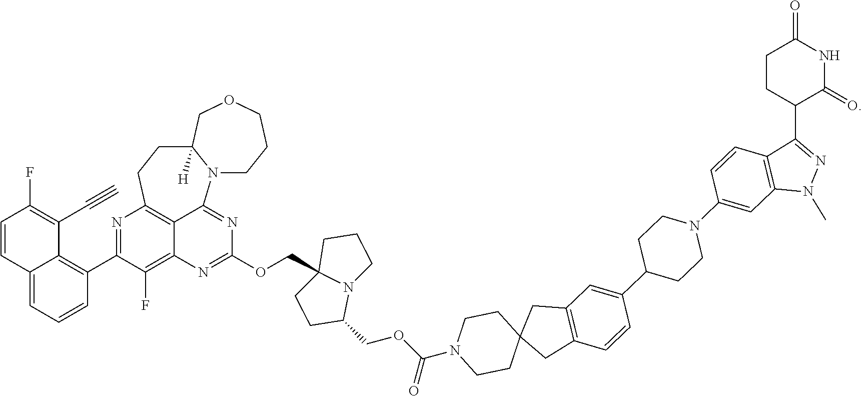 |
| 2-30 | 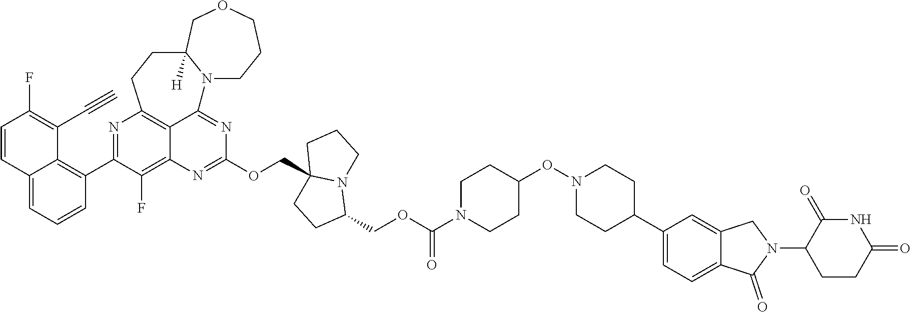 |
| 2-31 | 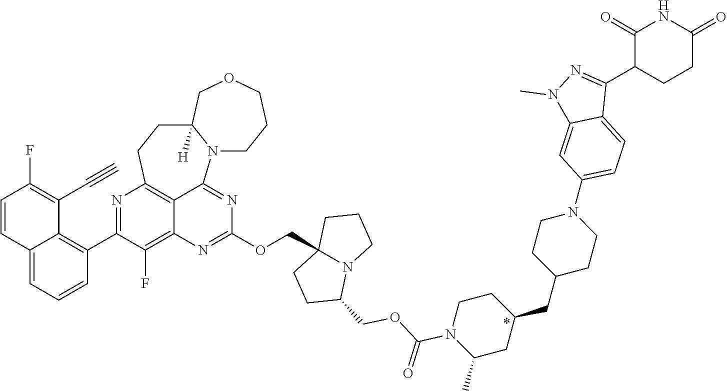 |
| 2-32 | 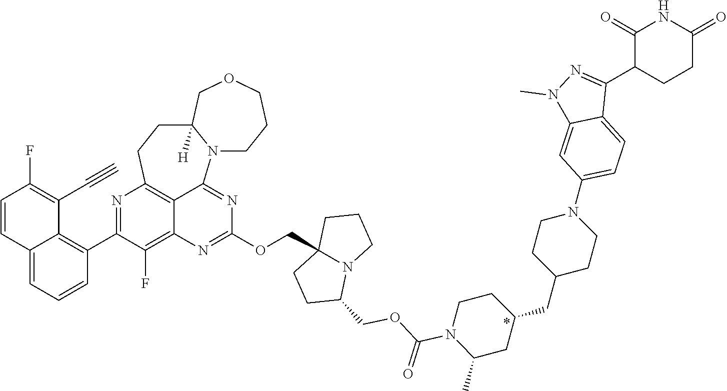 |
| 2-33 | 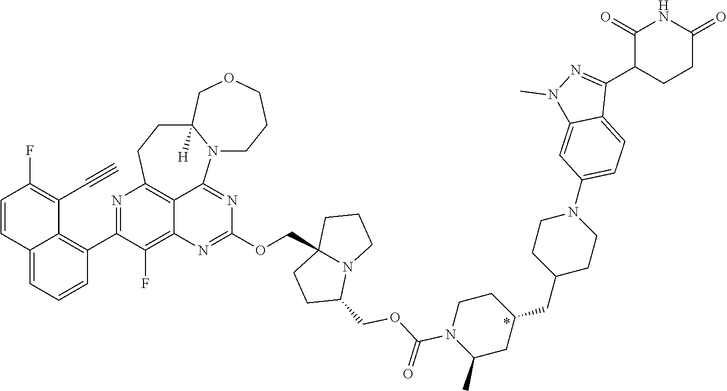 |
| 2-34 | 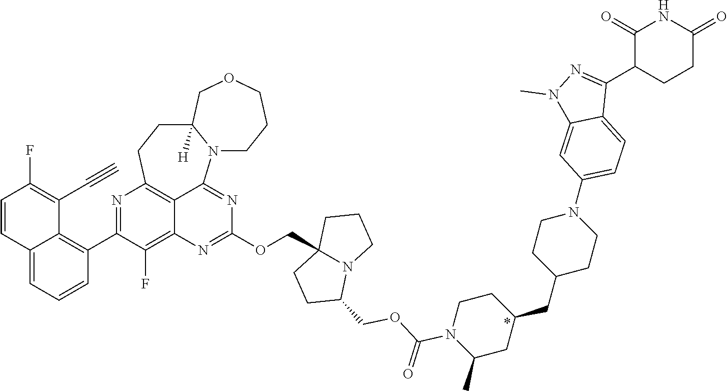 |
| 2-35 | 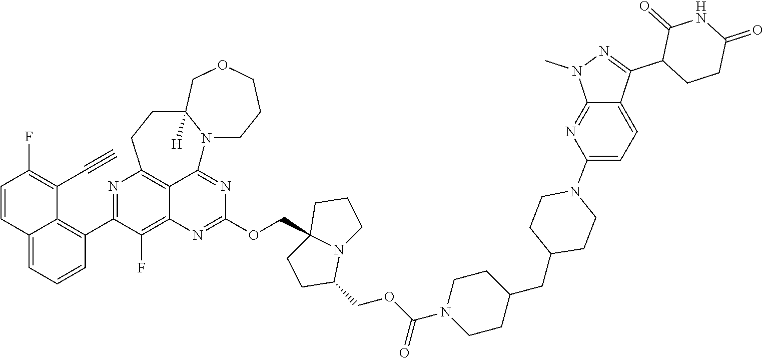 |
| 2-36 | 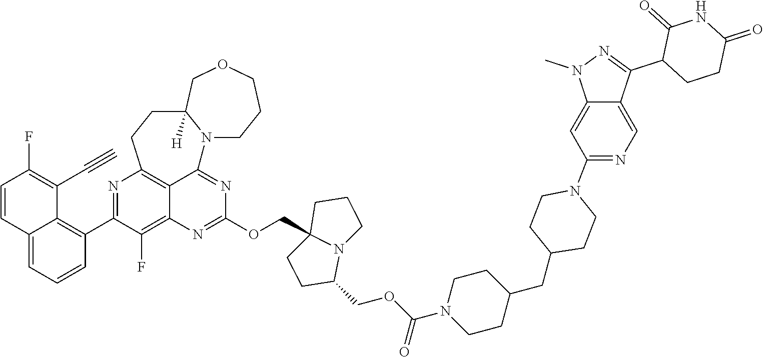 |
| 2-37 | 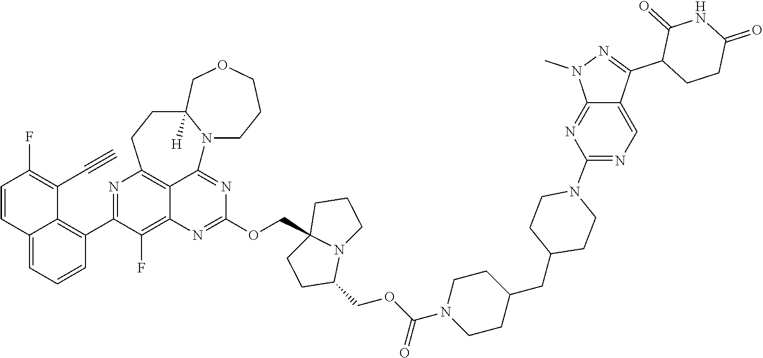 |
| 2-38 | 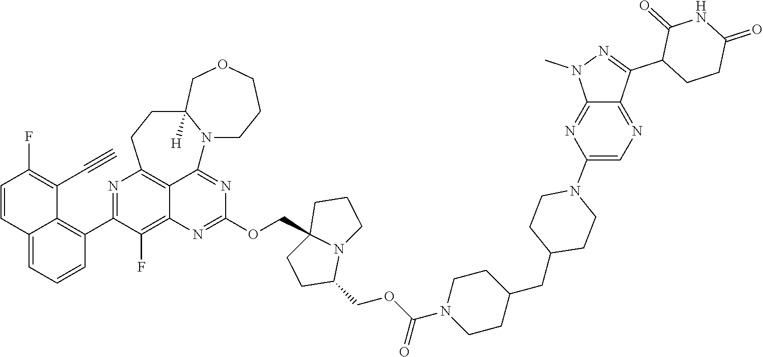 |
| 2-39 | 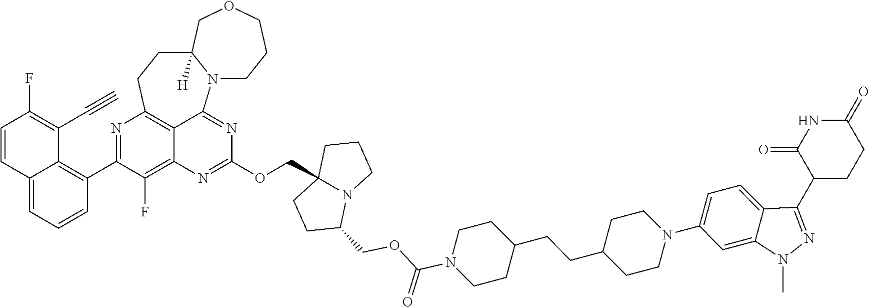 |
| 2-40 | 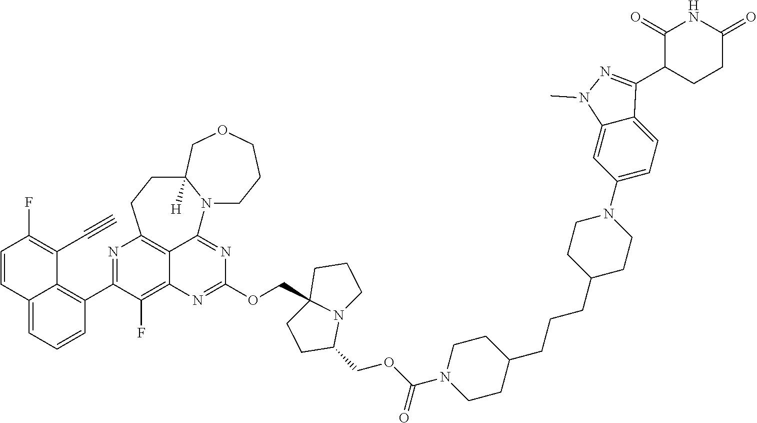 |
| 2-41 | 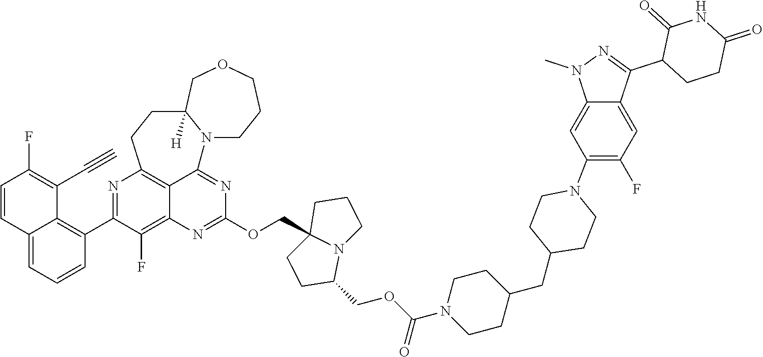 |
| 2-42 | 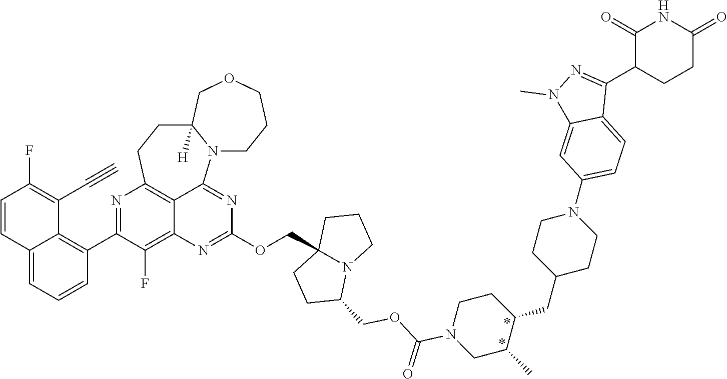 |
| 2-43 | 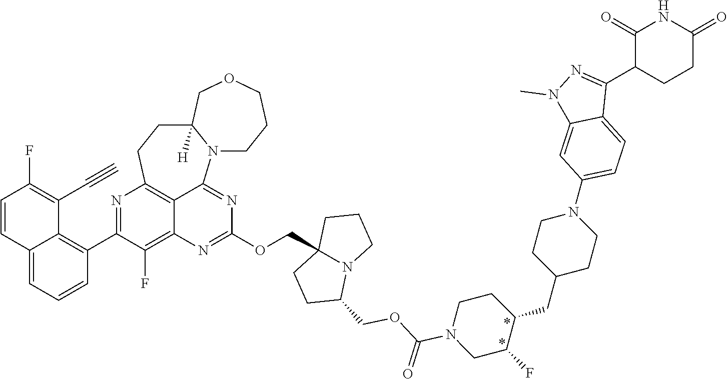 |
| 2-44 | 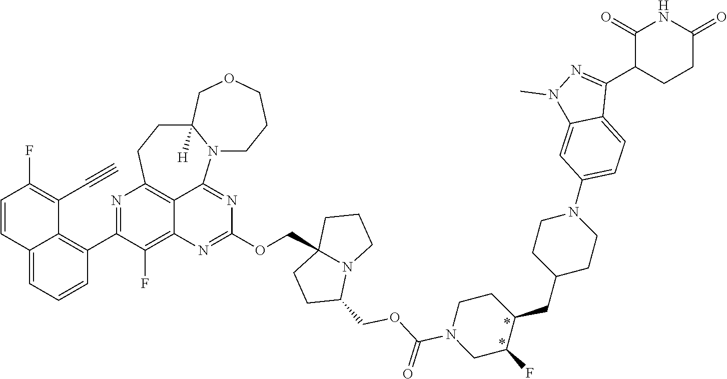 |
| 2-45 | 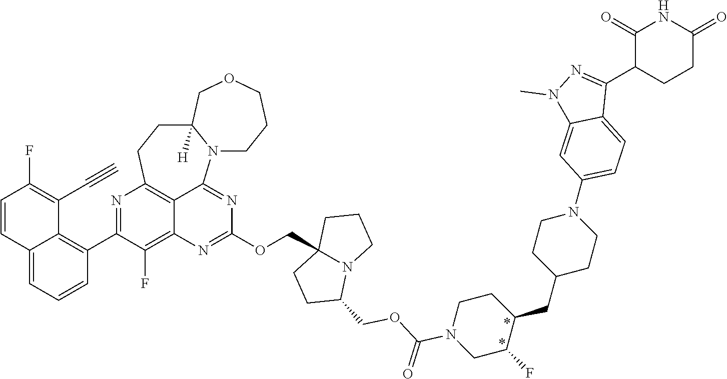 |
| 2-46 | 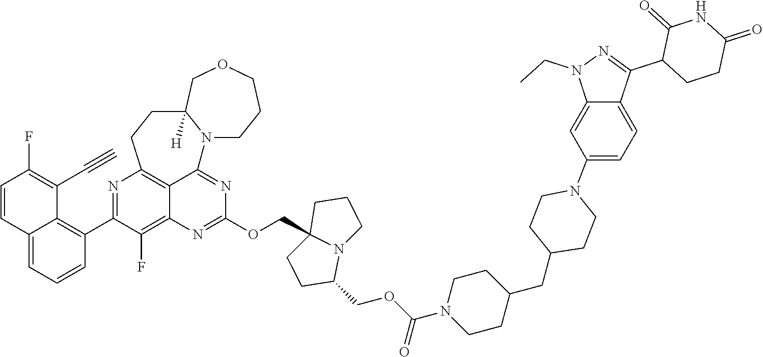 |
| 2-47 | 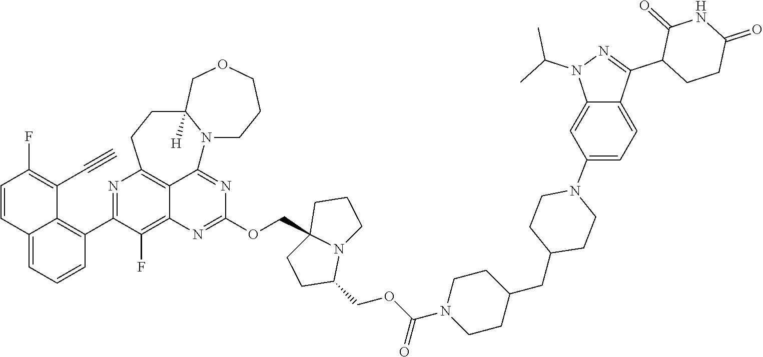 |
| 2-48 | 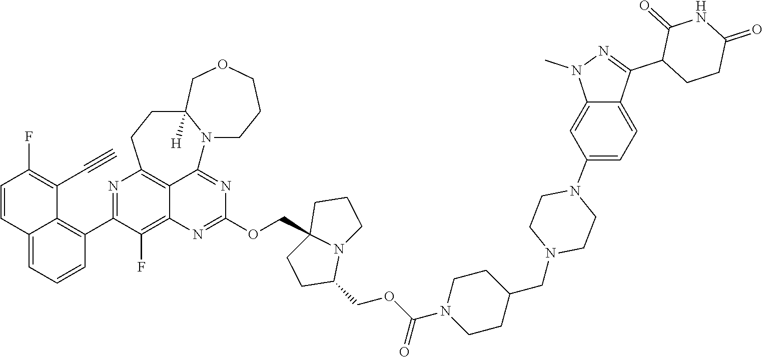 |
| 2-49 | 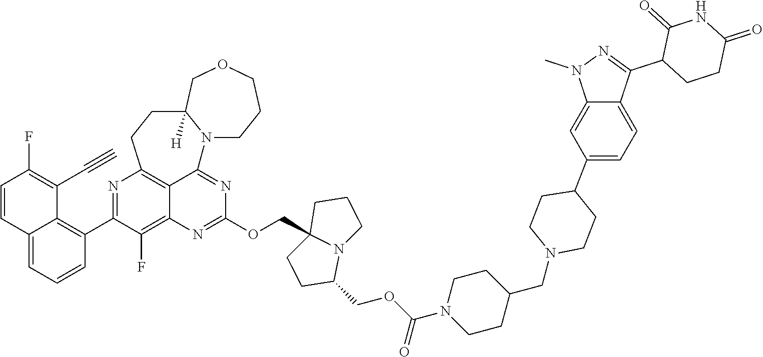 |
| 2-50 | 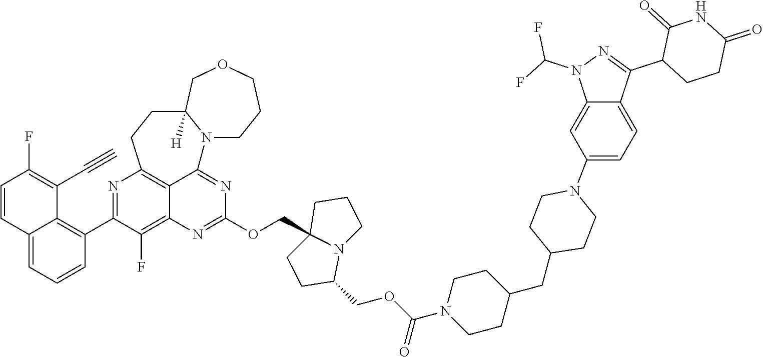 |
| 2-51 | 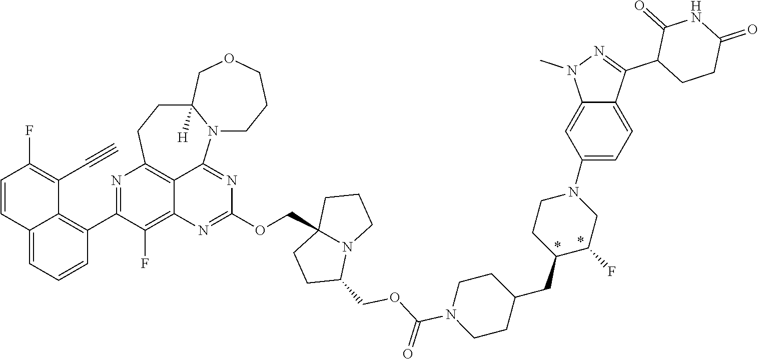 |
| 2-52 | 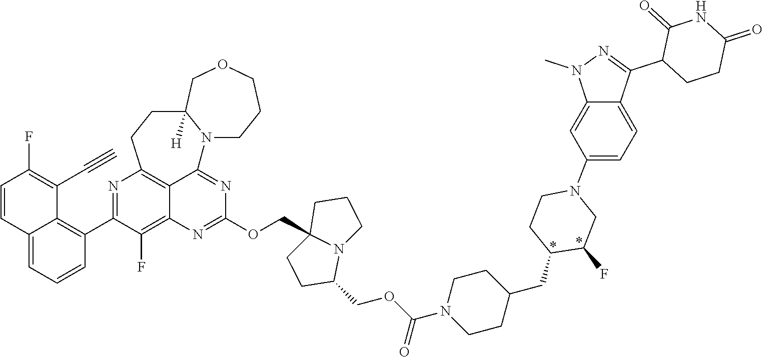 |
| 2-53 | 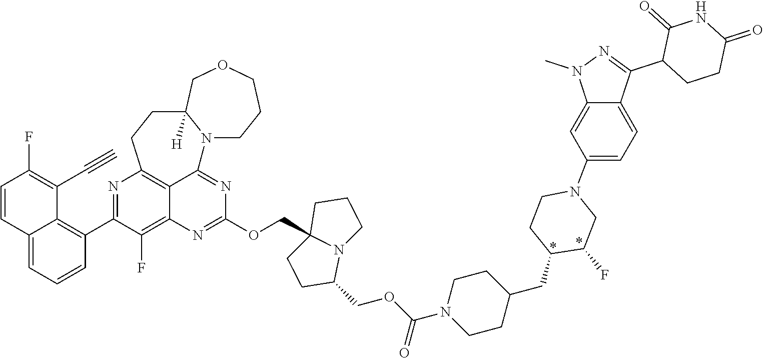 |
| 2-54 | 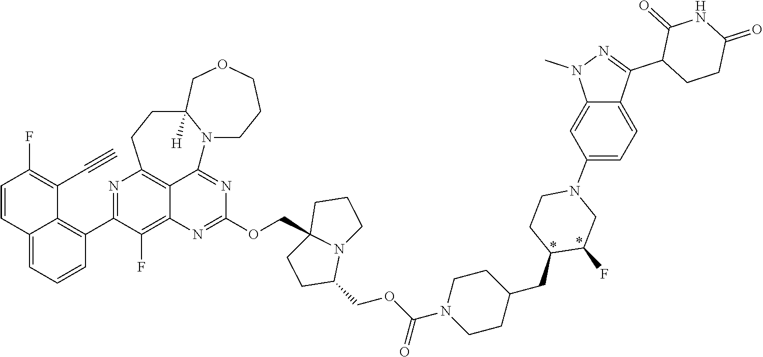 |
| 2-55 | 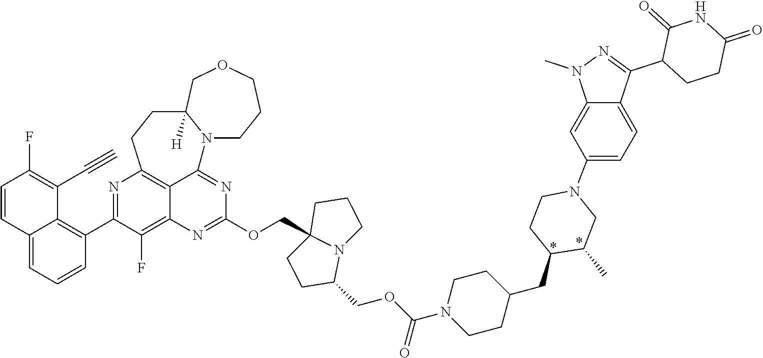 |
| 2-56 | 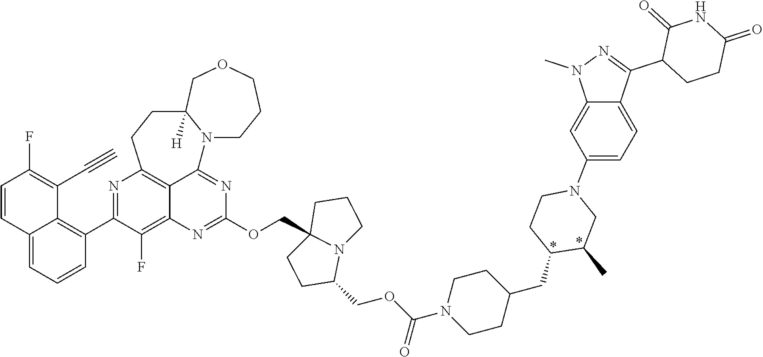 |
| 2-57 | 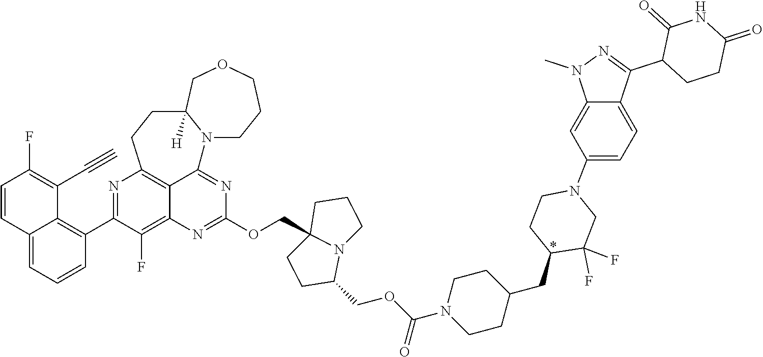 |
| 2-58 | 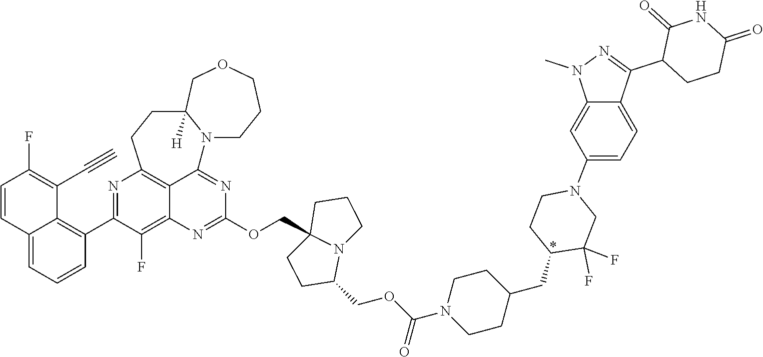 |
| 2-59 | 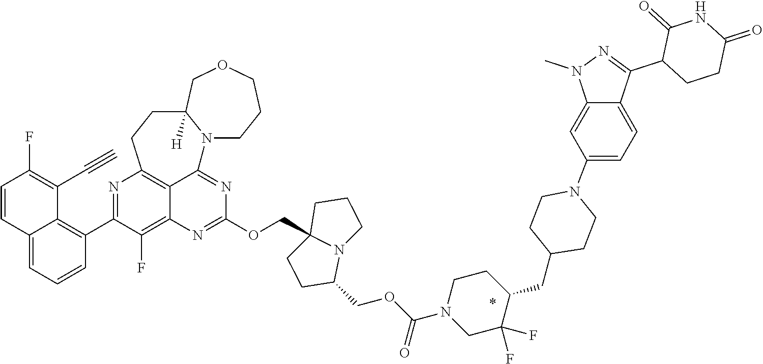 |
| 2-60 | 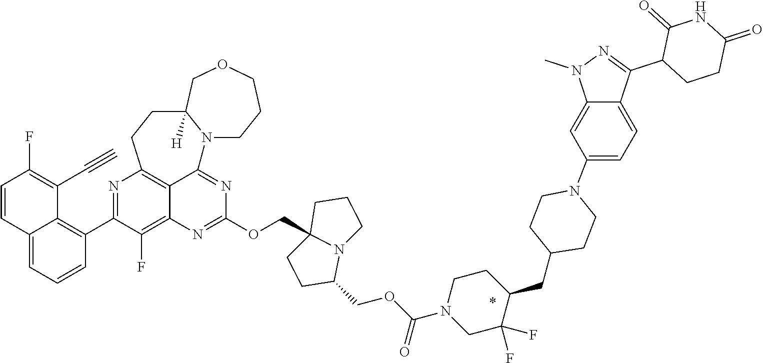 |
| 2-61 | 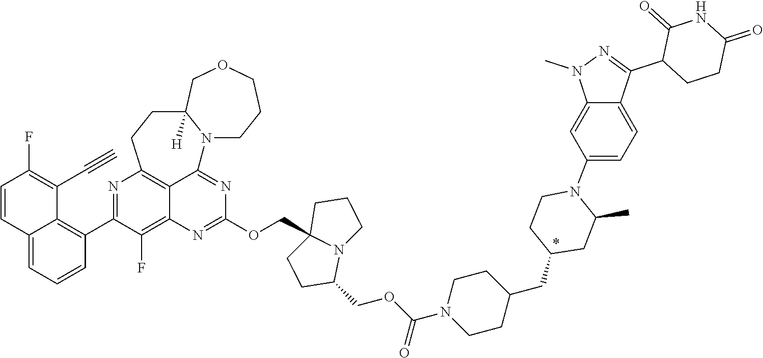 |
| 2-62 | 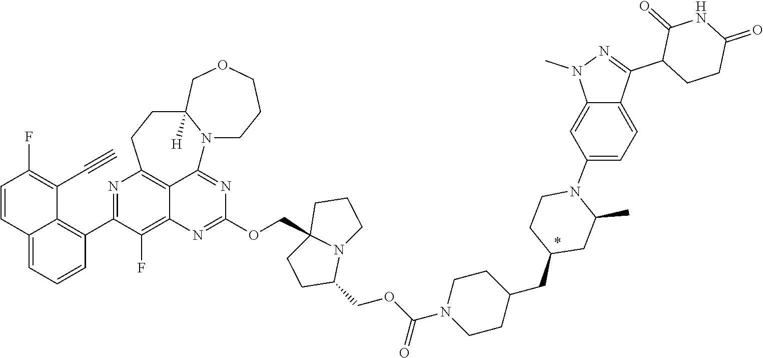 |
| 2-63 | 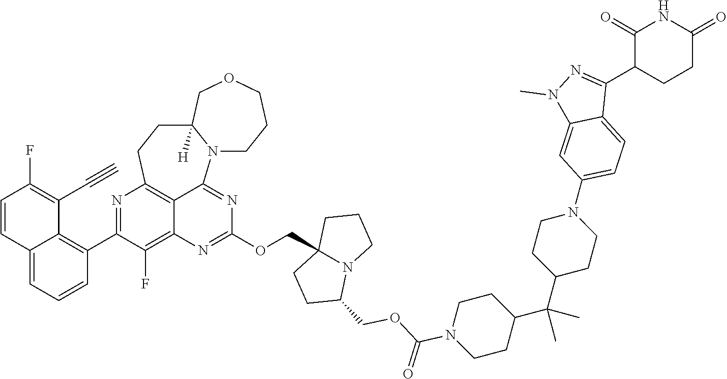 |
| 2-64 | 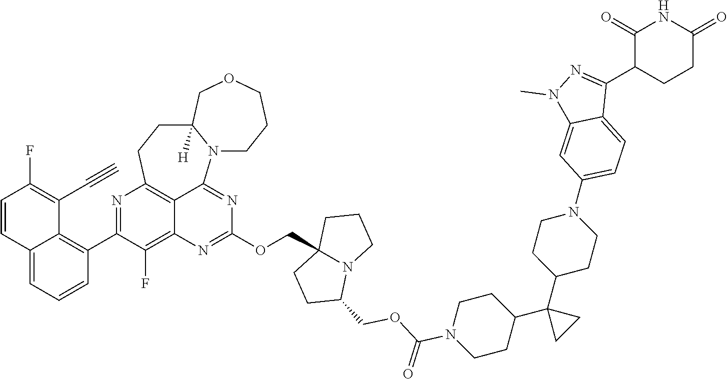 |
| 2-65 | 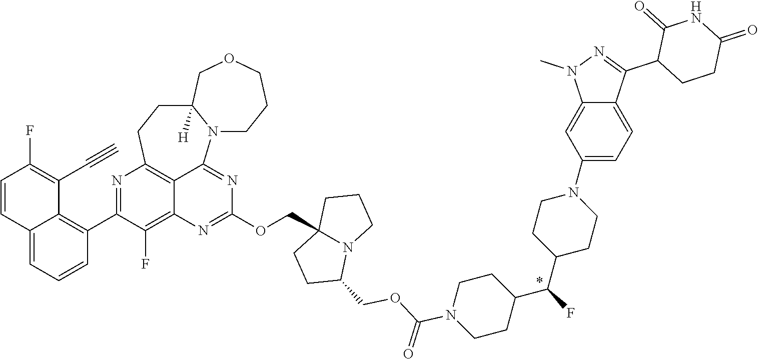 |
| 2-66 | 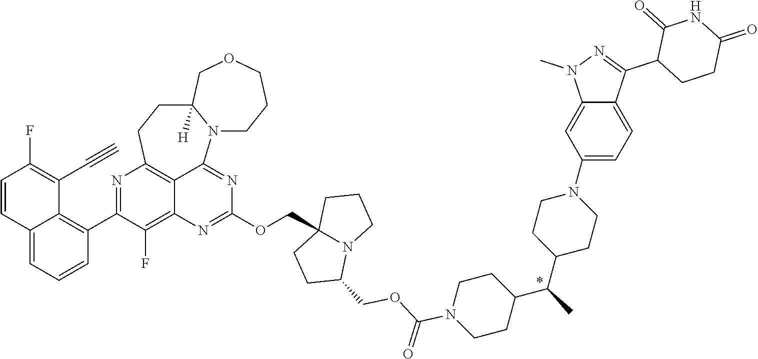 |
| 2-67 | 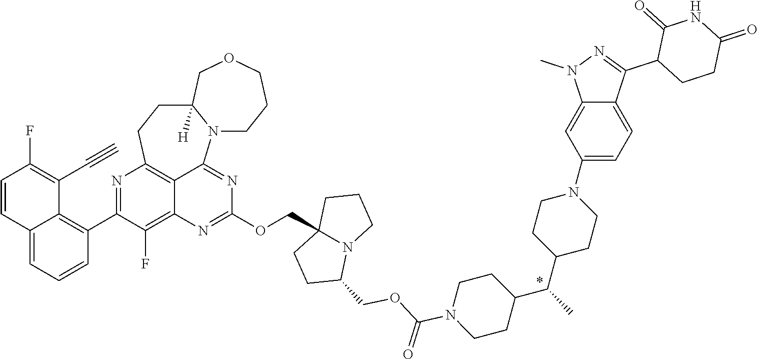 |
| 2-68 | 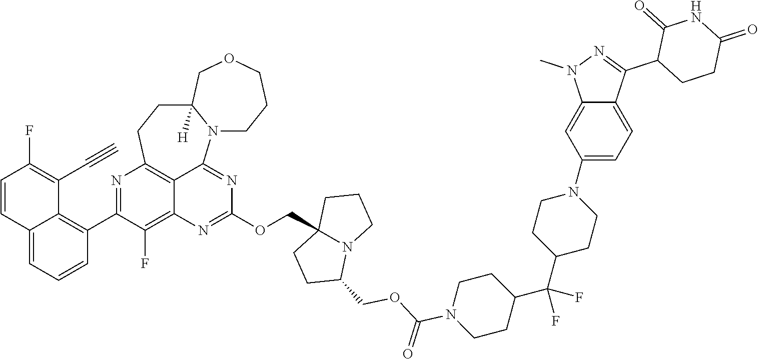 |
| 2-69 | 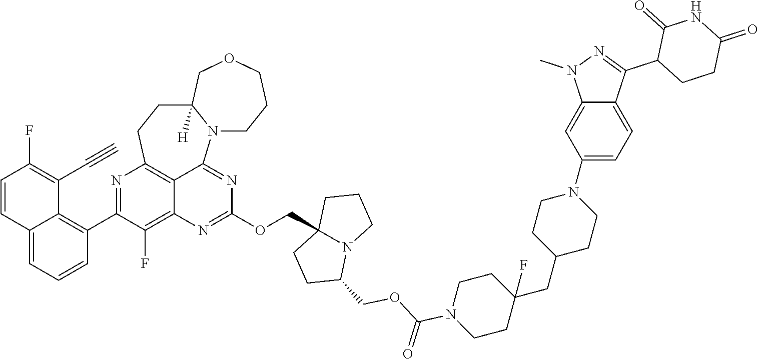 |
| 2-70 | 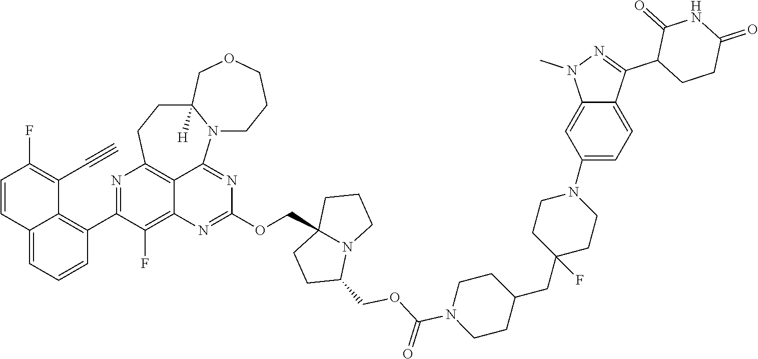 |
| 2-71 | 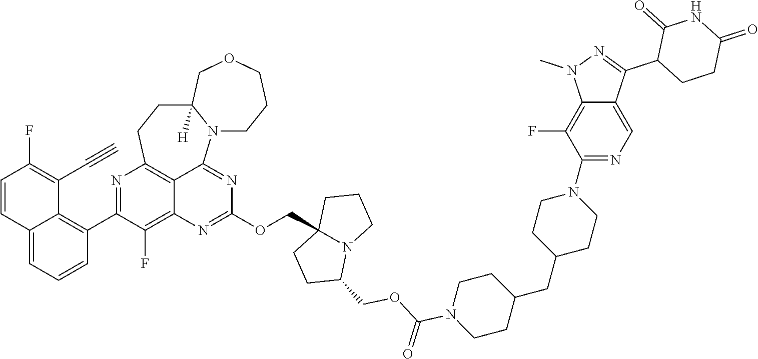 |
| 2-72 | 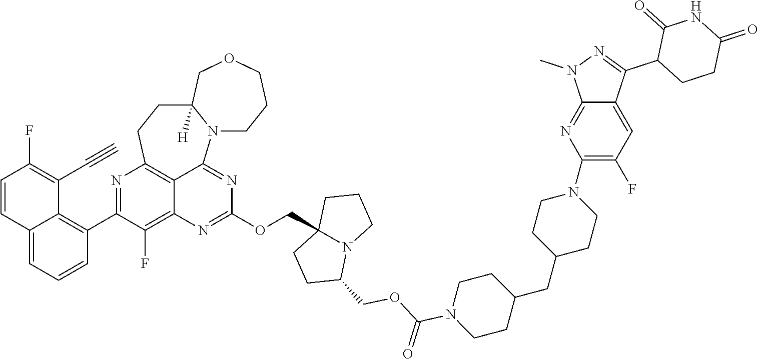 |
| 2-73 | 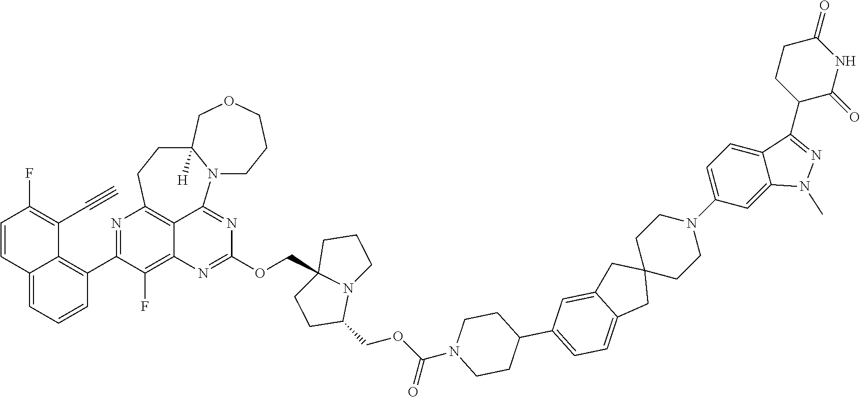 |
| 2-74 | 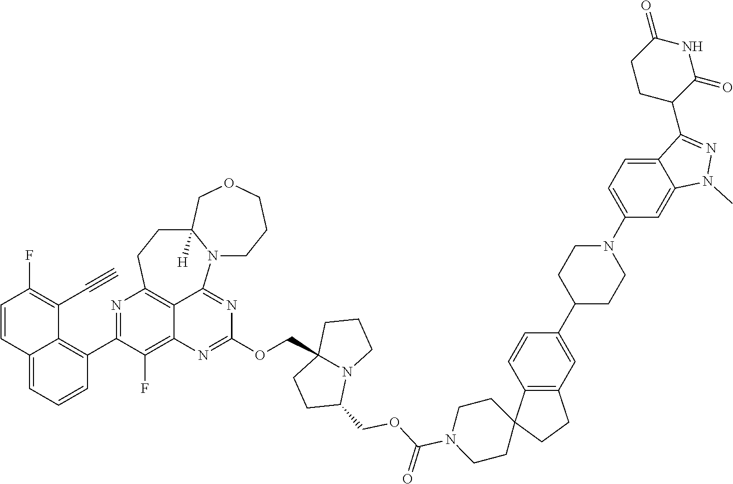 |
| 2-75 | 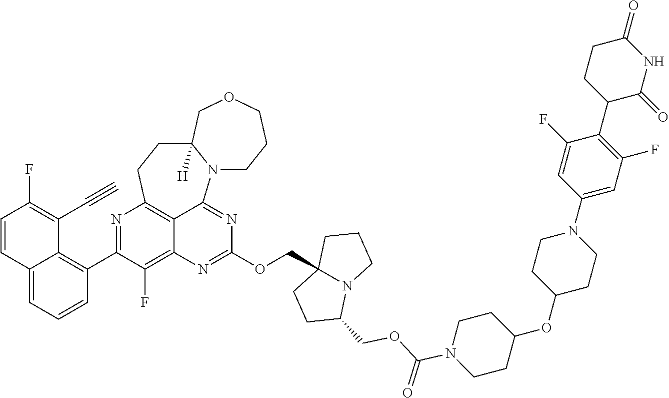 |
| 2-76 | 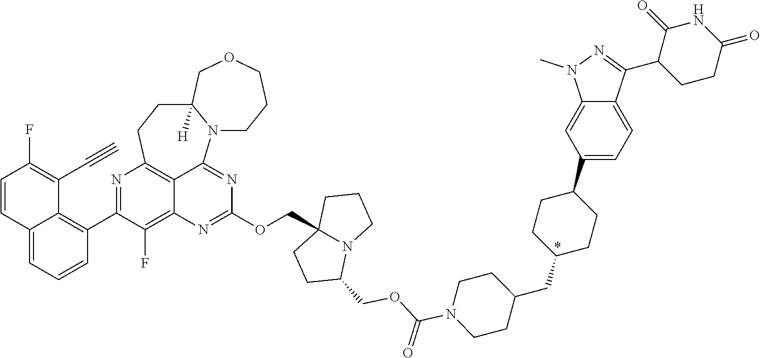 |
| 2-77 | 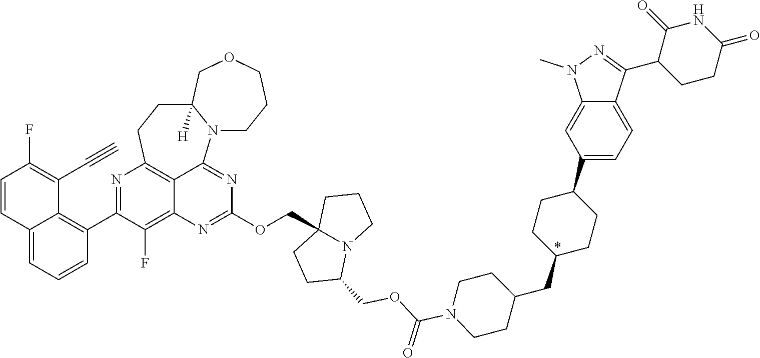 |
| 2-78 | 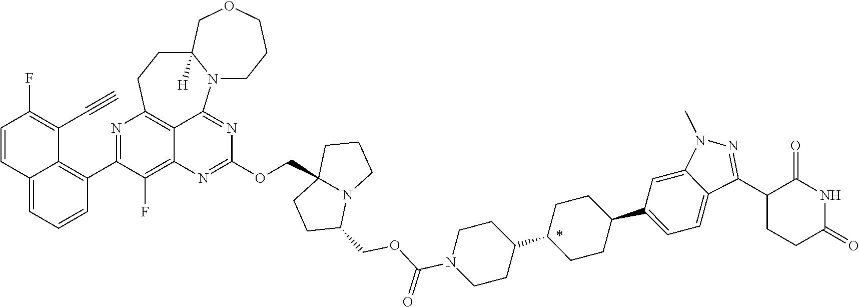 |
| 2-79 | 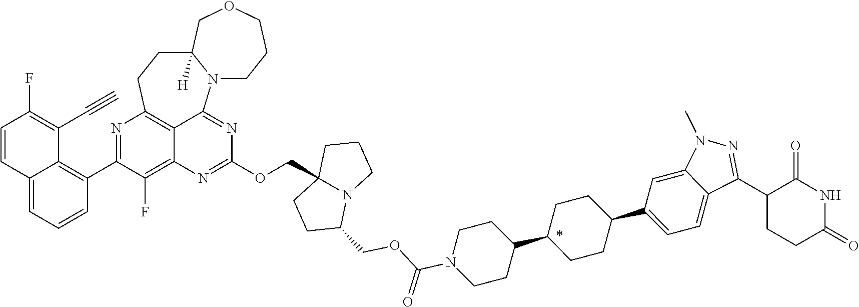 |
| 2-80 | 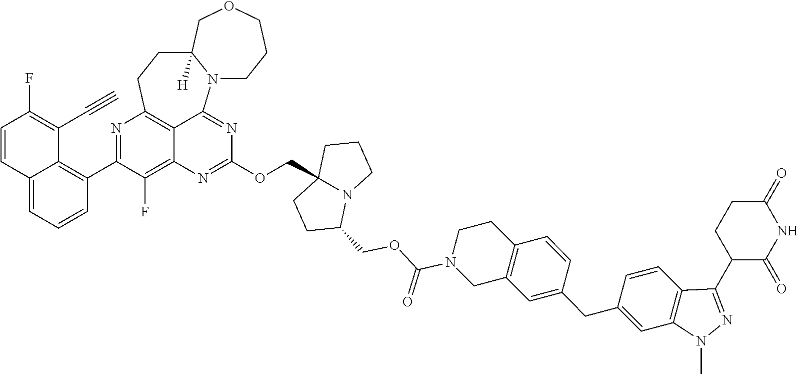 |
| 2-81 | 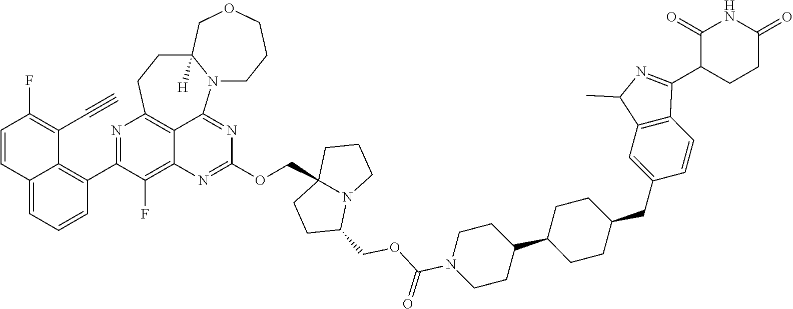 |
| 2-82 | 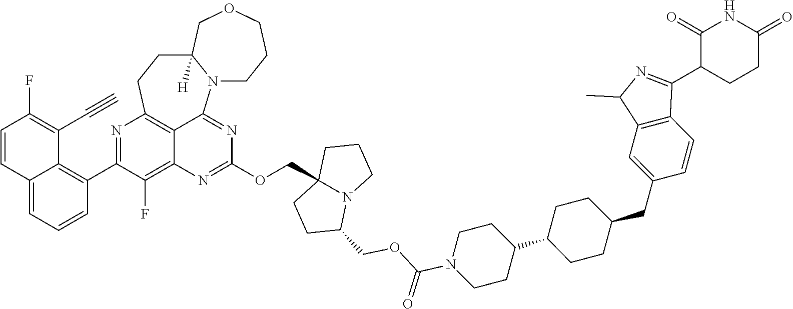 |

TABLE 3B2
Compounds

| | | | Changes to |
|---|---|---|---|
| | | | Procedure: |
| | | | Different |
| | | | Reagents/ |
| | | | Starting |
| Example | LCMS | ¹H-NMR (400 MHz, Methanol-d₄) | Materials |
| 2-2 | 1077.9 | δ 8.22-8.11 (m, 2H), 7.73-7.65 (m, 2H), 7.62 (d, J = | INT 9-2 |
| | | 8.4 Hz, 1H), 7.47 (t, J = 8.9 Hz, 1H), 7.08 (s, 2H), 5.09 | in step 1 |
| | | (dd, J = 13.3, 5.1 Hz, 1H), 4.77-4.62 (m, 3H), 4.61- | |
| | | 4.42 (m, 2H), 4.35 (d, J = 14.7 Hz, 4H), 4.18-3.81 (m, | |
| | | 7H), 3.81-3.45 (m, 6H), 3.42 (s, 2H), 3.22-3.04 (m, | |
| | | 5H), 2.94-2.71 (m, 4H), 2.50-2.20 (m, 7H), 2.19- | |
| | | 2.06 (m, 5H), 1.94 (s, 3H), 1.78-1.62 (m, 5H), 1.29- | |
| | | 1.12 (m, 2H) | |
| 2-3 | 1077.9 | δ 8.18-8.12 (m, 2H), 7.78-7.54 (m, 3H), 7.46 (s, 1H), | INT 10-1 |
| | | 7.07 (s, 2H), 4.82-4.73 (m, 2H), 4.78-4.57 (m, 4H), | in step 1 |
| | | 4.38 (d, J = 5.7 Hz, 5H), 4.12-3.80 (m, 7H), 3.76-3.56 | |
| | | (m, 5H), 3.50 (p, J = 1.7 Hz, 3H), 3.13 (p, J = 1.6 Hz, | |
| | | 3H), 2.97-2.70 (m, 4H), 2.57-2.01 (m, 11H), 1.94 (s, | |
| | | 2H), 1.88-1.70 (m, 4H), 1.58-1.30 (m, 6H) | |
| 2-4 | 1063.6 | δ 8.13 (d, J = 8.8 Hz, 2H), 7.76 (d, J = 8.9 Hz, 1H), | INT 6-1 |
| | | 7.73-7.62 (m, 2H), 7.52-7.44 (m 1H), 7.35 (s, 1H), 7.16 | in step 1 |
| | | (d, J = 8.9 Hz, 1H), 4.80-4.60 (m, 3H), 4.60-4.45 (m, | |
| | | 2H), 4.43-4.32 (m, 2H), 4.28 (s, 1H), 4.14-3.90 (m, | |
| | | 7H), 3.65-3.57 (m, 7H), 3.50 (p, J = 1.7, 2H), 3.51- | |
| | | 3.36 (m, 5H), 3.19-3.00 (m, 3H), 2.93-2.58 (m, 2H), | |
| | | 2.55-2.20 (m, 8H), 2.21-2.08 (m, 6H), 2.03-1.83 | |
| | | (m, 5H), 1.61-1.51 (m, 2H) | |
| 2-5 | 1026.9 | δ 8.19-8.13 (m, 2H), 7.75-7.63 (m, 2H), 7.47 (t, J = | INT 6-4 |
| | | 8.9 Hz, 1H), 7.16 (t, J = 8.5 Hz, 1H), 6.91-6.76 (m, | in step 1 |
| | | 2H), 4.81-4.72 (m, 1H), 4.64 (d, J = 12.3 Hz, 1H), | |
| | | 4.60-4.45 (m, 2H), 4.43-4.34 (m, 1H), 4.27 (s, 1H), 4.17- | |
| | | 3.90 (m, 5H), 3.88-3.63 (m, 6H), 3.63-3.53 (m, 3H), | |
| | | 3.50-3.37 (m, 3H), 3.26-3.12 (m, 3H), 3.13-3.04 | |
| | | (m, 3H), 2.81-2.60 (m, 2H), 2.44-2.03 (m, 12H), | |
| | | 2.03-1.89 (m, 3H), 1.83 (s, 2H), 1.50 (m, 4H) | |
| 2-6 | 1103.9 | δ 8.24-8.10 (m, 2H), 7.80-7.58 (m, 3H), 7.49 (t, J = | INT 11-1 |
| | | 8.9 Hz, 1H), 7.19-7.06 (m, 2H), 5.12 (dd, J = 13.3, 5.2 | in step 1 |
| | | Hz, 1H), 4.82-4.71 (m, 1H), 4.72-4.32 (m, 6H), | |
| | | 4.32-4.22 (m, 1H), 4.18-3.82 (m, 5H), 3.79-3.41 (m, | |
| | | 11H), 3.26-3.00 (m, 4H), 2.92 (ddd, J = 18.3, 13.4, 5.3 | |
| | | Hz, 1H), 2.85-2.72 (m, 1H), 2.57-1.25 (m, 27H). | |
| 2-7 | 1075.9 | δ 8.23-8.12 (m, 2H), 7.82-7.60 (m, 3H), 7.49 (t, J = | INT 11-2 |
| | | 8.9 Hz, 1H), 7.14-7.07 (m, 2H), 5.12 (dd, J = 13.3, 5.1 | in step 1 |
| | | Hz, 1H), 4.81-7.71 (m, 1H), 4.71-4.54 (m, 2H), 4.52- | |
| | | 4.32 (m, 4H), 4.31-4.19 (m, 1H), 4.18-3.82 (m, 9H), | |
| | | 3.81-3.67 (m, 3H), 3.65-3.40 (m, 5H), 3.25-3.06 | |
| | | (m, 4H), 2.92 (ddd, J = 18.4, 13.5, 5.4 Hz, 2H), 2.85- | |
| | | 2.74 (m, 1H), 2.62-1.88 (m, 19H), 1.72-1.54 (m, 2H). | |
| 2-8 | 1103.9 | δ 8.23-8.11 (m, 2H), 7.81-7.61 (m, 3H), 7.58-7.39 | INT 10-2 |
| | | (m, 1H), 7.18-7.08 (m, 2H), 5.12 (dd, J = 13.3, 5.2 Hz, | in step 1 |
| | | 1H), 4.77 (d, J = 12.3 Hz, 1H), 4.68 (dd, J = 12.3, 5.8 | |
| | | Hz, 1H), 4.63-4.46 (m, 2H), 4.45-4.24 (m, 3H), 4.23- | |
| | | 3.68 (m, 7H), 3.65-3.04 (m, 21H), 2.92 (ddd, J = | |
| | | 18.4, 13.5, 5.4 Hz, 1H), 2.84-2.74 (m, 1H), 2.56-1.93 | |
| | | (m, 12H), 1.90-1.62 (m, 6H), 1.59-1.35 (m, 2H). | |
| 2-9 | 1075.9 | δ 8.21-8.08 (m, 2H), 7.79-7.65 (m, 2H), 7.59 (d, J = | INT 10-3 |
| | | 8.9 Hz, 1H), 7.53-7.41 (m, 1H), 6.56-6.44 (m, 2H), | in step 1 |
| | | 5.10 (dd, J = 13.3, 5.2 Hz, 1H), 4.81-4.71 (m, 1H), | |
| | | 4.69-4.45 (m, 3H), 4.44-4.21 (m, 3H), 4.19-3.66 (m, | |
| | | 13H), 3.65-3.01 (m, 9H), 2.91 (ddd, J = 18.4, 13.5, | |
| | | 5.4 Hz, 1H), 2.84-2.71 (m, 1H), 2.67-1.91 (m, 18H), | |
| | | 1.90-1.69 (m, 2H), 1.57-1.37 (m, 2H). | |
| 2-10 | 1103.9 | δ 8.28-8.08 (m, 2H), 7.81-7.59 (m, 3H), 7.50 (t, J = | INT 11-3 |
| | | 8.9 Hz, 1H), 7.18-7.06 (m, 2H), 5.12 (dd, J = 13.3, 5.2 | in step 1 |
| | | Hz, 1H), 4.81-3.84 (m, 12H), 3.81-3.37 (m, 12H), | |
| | | 3.28-3.02 (m, 4H), 2.98-2.73 (m, 2H), 2.56-1.89 | |
| | | (m, 19H), 1.81-1.42 (m, 8H). | |
| 2-11 | 1102.9 | δ 8.25-8.05 (m, 2H), 7.92-7.80 (m, 1H), 7.78-7.38 | INT 6-2 |
| | | (m, 4H), 7.33-7.19 (m, 1H), 4.82-4.64 (m, 2H), | in step 1 |
| | | 4.64-4.46 (m, 2H), 4.45-4.19 (m, 3H), 4.18-3.00 (m, | |
| | | 28H), 2.95-2.66 (m, 2H), 2.60-1.72 (m, 18H), 1.70- | |
| | | 1.30 (m, 4H). | |
| 2-12 | 1048.5 | δ 8.25-8.04 (m, 2H), 7.80-7.60 (m, 3H), 7.46 (t, J = | INT 17-2 |
| | | 8.9 Hz, 1H), 7.17-7.09 (s, 2H), 5.11 (dd, J = 13.3, 5.1 | in step 1 |
| | | Hz, 1H), 4.77-4.65 (m, 2H), 4.59-4.30 (m, 6H), 4.16- | |
| | | 3.81 (m, 8H), 3.75-3.66 (m, 2H), 3.63-3.55 (m, 1H), | |
| | | 3.46-3.36 (m, 1H), 3.25-3.05 (m, 3H), 2.95-2.66 | |
| | | (m, 6H), 2.52-1.95 (m, 13H), 1.84-1.59 (m, 5H), | |
| | | 1.39-1.12 (m, 7H). | |
| 2-13 | 1047.6 | δ 8.20-8.06 (m, 2H), 7.92 (d, J = 8.8 Hz, 1H), 7.79- | INT 14-2 |
| | | 7.63 (m, 3H), 7.45 (t, J = 8.8 Hz, 1H), 7.33 (dd, J = 8.9, | in step 1 |
| | | 1.8 Hz, 1H), 4.82-4.64 (m, 2H), 4.60-4.49 (m, 2H), | |
| | | 4.42 (dd, J = 9.6, 5.1 Hz, 1H), 4.37-4.27 (m, 2H), | |
| | | 4.27-4.12 (m, 2H), 4.12-3.81 (m, 8H), 3.81-3.67 (m, 3H), | |
| | | 3.66-3.49 (m, 3H), 3.45-3.37 (m, 2H), 3.25-3.05 | |
| | | (m, 2H), 2.86-2.71 (m, 4H), 2.55-1.95 (m, 16H), 1.83- | |
| | | 1.65 (m, 4H), 1.52-1.61 (m, 1H), 1.44-1.34 (m, 1H), | |
| | | 1.29-1.14 (m, 2H). | |
| 2-14 | 1062.6 | δ 8.26-8.08 (m, 2H), 7.75-7.67 (m, 3H), 7.50-7.46 | INT 17-5 |
| | | (m, 1H), 7.25-7.20 (m, 2H), 5.11 (dd, J = 13.3, 5.2 Hz, | in step 1 |
| | | 1H), 4.81-4.64 (m, 2H), 4.62-4.21 (m, 6H), 4.20- | |
| | | 3.66 (m, 10H), 3.57-3.63 (m, 1H), 3.45-3.38 (m, 1H), | |
| | | 3.26-2.99 (m, 4H), 2.95-2.71 (m, 4H), 2.52-2.32 | |
| | | (m, 5H), 2.29-2.04 (m, 8H), 2.01-1.94 (m, 1H), 1.87- | |
| | | 1.79 (m, 2H), 1.74-1.52 (m, 4H), 1.41-1.27 (m, 2H), | |
| | | 1.24-1.13 (m, 2H), 1.12-0.99 (m, 2H). | |
| 2-15 | 1061.6 | δ 8.24-8.08 (m, 2H), 8.01-7.90 (m, 1H), 7.84 (d, J = | INT 14-3 |
| | | 2.4 Hz, 1H), 7.77-7.65 (m, 2H), 7.47 (td, J = 8.9, 2.2 | in step 1 |
| | | Hz, 1H), 7.36 (dt, J = 9.3, 2.5 Hz, 1H), 4.80-4.68 (m, | |
| | | 2H), 4.61-4.25 (m, 5H), 4.18-3.81 (m, 10H), 3.80- | |
| | | 3.59 (m, 7H), 3.47-3.27 (m, 1H), 3.25-3.06 (m, 2H), | |
| | | 2.87-2.71 (m, 4H), 2.56-2.30 (m, 6H), 2.27-2.05 | |
| | | (m, 9H), 2.02-1.83 (m, 2H), 1.80-1.55 (m, 5H), 1.30 | |
| | | (d, J = 6.9 Hz, 2H), 1.10 (q, J = 12.2, 11.8 Hz, 2H). | |
| 2-16 | 1062.6 | δ 8.11 (dd, J = 9.2, 5.6 Hz, 2H), 7.67 (ddd, J = 12.8, 8.4, | INT 14-1 |
| | | 5.9 Hz, 3H), 7.44 (q, J = 8.8 Hz, 1H), 7.10-6.97 (m, | in step 1 |
| | | 2H), 4.80-4.63 (m, 3H), 4.62-4.50 (m, 2H), 4.49- | |
| | | 4.25 (m, 3H), 4.14-3.91 (m, 8H), 3.90-3.58 (m, 8H), | |
| | | 3.45-3.34 (m, 2H), 3.26-2.93 (m, 8H), 2.84-2.67 | |
| | | (m, 3H), 2.51-2.36 (m, 4H), 2.35-2.05 (m, 10H), | |
| | | 2.02-1.89 (m, 3H), 1.63-1.42 (m, 3H). | |
| 2-17 | 1076.6 | δ 8.17-8.01 (m, 2H), 8.73-8.57 (m, 3H), 7.47-7.40 | INT 15-1 |
| | | (m, 1H), 6.99-6.84 (m, 2H), 4.77-4.65 (m, 2H), | in step 1 |
| | | 4.60-4.45 (m, 2H), 4.37-4.25 (m, 3H), 4.14-3.79 (m, | |
| | | 10H), 3.78-3.54 (m, 6H), 3.54-3.35 (m, 5H), 3.26- | |
| | | 3.01 (m, 5H), 2.84-2.67 (m, 3H), 2.49-1.94 (m, 13H), | |
| | | 1.65-1.45 (m, 4H), 1.24 (s, 3H). | |
| 2-18 | 1077.6 | δ 8.23-8.08 (m, 2H), 7.76-7.57 (m, 3H), 7.53-7.40 | INT 18-1 |
| | | (m, 1H), 7.15-7.02 (m, 2H), 5.12 (dd, J = 13.0, 4.8 Hz, | in step 1 |
| | | 1H), 4.82-4.65 (m, 3H), 4.61-4.53 (m, 2H), 4.49- | |
| | | 4.30 (m, 4H), 4.17-3.80 (m, 7H), 3.78-3.57 (m, 5H), | |
| | | 3.50-3.36 (m, 4H), 3.26-3.05 (m, 6H), 2.96-2.75 | |
| | | (m, 3H), 2.52-2.07 (m, 12H), 2.02-1.96 (m, 2H), | |
| | | 1.78-1.61 (m, 4H), 1.32-1.21 (m, 4H). | |
| 2-19 | 1076.6 | δ 8.20-8.12 (m, 2H), 7.82-7.62 (m, 3H), 7.48 (t, J = | INT 17-7 |
| | | 8.9 Hz, 1H), 7.23 (d, J = 8.7 Hz, 2H), 5.11 (dd, J = 13.3, | in step 1 |
| | | 5.1 Hz, 1H), 4.80-4.63 (m, 2H), 4.63-4.19 (m, 6H), | |
| | | 4.19-3.65 (m, 10H), 3.64-3.56 (m, 1H), 3.46-3.35 | |
| | | (m, 1H), 3.26-2.97 (m, 4H), 2.95-2.67 (m, 4H), 2.52- | |
| | | 2.31 (m, 5H), 2.30-2.02 (m, 8H), 2.01-1.93 (m, 2H), | |
| | | 1.89-1.88 (m, 2H), 1.76-1.57 (m, 2H), 1.53-1.45 | |
| | | (m, 1H), 1.42-1.22 (m, 7H), 1.09-0.98 (m, 2H). | |
| 2-20 | 1077.6 | δ 8.25-8.08 (m, 2H), 7.78-7.64 (m, 2H), 7.59-7.41 | INT 20-1 |
| | | (m, 2H), 7.43-7.24 (m, 2H), 5.49-5.33 (m, 1H), 4.81- | in step 1 |
| | | 4.63 (m, 2H), 4.63-4.26 (m, 4H), 4.23-3.92 (m, 6H), | |
| | | 3.92-3.82 (m, 1H), 3.77-3.74 (m, 1H), 3.74-3.65 | |
| | | (m, 4H), 3.65-3.57 (m, 1H), 3.50-3.48 (m, 3H), 3.47- | |
| | | 3.38 (m, 2H), 3.27-3.03 (m, 2H), 3.01-2.75 (m, 5H), | |
| | | 2.51-2.04 (m, 14H), 2.04-1.96 (m, 1H), 1.94-1.84 | |
| | | (m, 1H), 1.84-1.53 (m, 5H), 1.37-1.25 (m, 2H), | |
| | | 1.20-1.05 (m, 2H). | |
| 2-21 | 1079.6 | δ 8.14 (t, J = 7.4 Hz, 2H), 7.76-7.62 (m, 2H), 7.53- | INT 20-2 |
| | | 7.36 (m, 2H), 7.35-7.21 (m, 2H), 5.46-5.35 (m, 1H), | in step 1 |
| | | 4.81-4.63 (m, 2H), 4.63-4.48 (m, 2H), 4.46-4.25 | |
| | | (m, 2H), 4.17-3.67 (m, 14H), 3.65-3.51 (m, 4H), 3.47 | |
| | | (s, 3H), 3.12-3.04 (m, 1H), 3.01-2.89 (m, 1H), 2.89- | |
| | | 2.75 (m, 2H), 2.56-1.98 (m, 19H), 1.94-1.79 (m, 2H), | |
| | | 1.65-1.50 (m, 2H). | |
| 2-22 | 1064.5 | δ 8.22-8.11 (m, 2H), 7.77-7.67 (m, 2H), 7.60-7.43 | INT 17-8 |
| | | (m, 4H), 5.16 (dd, J = 13.3, 5.2 Hz, 1H), 4.77 (d, J = | in step 1 |
| | | 12.3 Hz, 1H), 4.73-4.64 (m, 1H), 4.62-4.43 (m, 4H), | |
| | | 4.43-4.25 (m, 2H), 4.17-3.91 (m, 4H), 3.91-3.56 | |
| | | (m, 10H), 3.48-3.40 (m, 1H), 3.30-3.16 (m, 5H), 3.13- | |
| | | 3.04 (m, 1H), 3.01-2.86 (m, 1H), 2.86-2.75 (m, 1H), | |
| | | 2.60-1.97 (m, 16H), 1.93-1.74 (m, 4H), 1.62-1.46 | |
| | | (m, 2H). | |
| 2-23 | 1077.6 | δ 8.13 (s, 2H), 7.94 (dd, J = 8.8, 2.6 Hz, 1H), 7.80 (s, | INT 22-1 |
| | | 1H), 7.78-7.63 (m, 2H), 7.45 (t, J = 9.0 Hz, 1H), 7.34 | in step 1 |
| | | (d, J = 8.8 Hz, 1H), 4.81-4.64 (m, 2H), 4.60-4.46 (m, | |
| | | 2H), 4.43 (dd, J = 9.7, 5.1 Hz, 1H), 4.38-4.24 (m, 2H), | |
| | | 4.12 (dd, J = 14.1, 3.5 Hz, 1H), 4.07 (s, 3H), 4.04-3.92 | |
| | | (m, 2H), 3.92-3.80 (m, 5H), 3.79-3.58 (m, 7H), 3.55- | |
| | | 3.36 (m, 2H), 3.29-3.03 (m, 1H), 2.87-2.68 (m, 2H), | |
| | | 2.58-2.29 (m, 6H), 2.29-1.95 (m, 12H), 1.79 (t, J = | |
| | | 12.3 Hz, 2H), 1.71-1.44 (m, 6H). | |
| 2-24 | 1102.9 | δ 8.30-8.04 (m, 2H), 7.91-7.80 (m, 1H), 7.79-7.64 | INT 6-5 |
| | | (m, 2H), 7.63-7.41 (m, 2H), 7.37-7.15 (m, 1H), | in step 1 |
| | | 4.81-4.46 (m, 5H), 4.44-3.67 (m, 16H), 3.65-3.56 (m, | |
| | | 2H), 3.54-3.36 (m, 5H), 3.25-3.03 (m, 3H), 2.91- | |
| | | 2.67 (m, 3H), 2.61-1.90 (m, 18H), 1.86-1.68 (m, 3H), | |
| | | 1.67-1.43 (m, 4H). | |
| 2-25 | 1062.6 | δ 8.20-8.10 (m, 2H), 7.89 (d, J = 8.9 Hz, 1H), 7.76- | INT 23-1 |
| | | 7.64 (m, 3H), 7.47 (td, J = 9.0, 2.7 Hz, 1H), 7.31 (dd, | in step 1 |
| | | J = 8.9, 2.1 Hz, 1H), 4.80-4.65 (m, 2H), 4.65-4.41 (m, | |
| | | 2H), 4.40-4.23 (m, 2H), 4.22-3.81 (m, 12H), 3.81- | |
| | | 3.67 (m, 4H), 3.67-3.50 (m, 3H), 3.48-3.36 (m, 1H), | |
| | | 3.27-3.03 (m, 2H), 2.87 (t, J = 6.7 Hz, 2H), 2.83-2.70 | |
| | | (m, 2H), 2.52-2.31 (m, 4H), 2.31-1.92 (m, 10H), 1.92- | |
| | | 1.81 (m, 1H), 1.72-1.79 (m, 1H), 1.65 (q, J = 12.4 | |
| | | Hz, 4H), 1.35-1.23 (m, 2H), 1.10 (q, J = 11.9 Hz, 2H). | |
| 2-26 | 1098.9 | δ 8.16-7.82 (m, 3H), 7.73-7.51 (m, 3H), 7.47-7.17 | INT 25-1 |
| | | (m, 3H), 7.13-6.97 (m, 1H), 4.82-4.20 (m, 11H), | in step 1 |
| | | 4.16-3.35 (m, 18H), 3.26-2.96 (m, 4H), 2.94-2.59 (m, | |
| | | 5H), 2.59-1.87 (m, 15H) | |
| 2-27 | 1077.6 | δ 8.19-8.09 (m, 2H), 7.79-7.63 (m, 3H), 7.52-7.38 | INT 26-1 |
| | | (m, 1H), 7.33-7.21 (m, 1H), 7.19-7.10 (m, 1H), 4.82- | in step 1. |
| | | 4.75 (m, 1H), 4.68-4.62 (m, 1H), 4.62-4.52 (m, 2H), | After the |
| | | 4.52-4.41 (m, 2H), 4.40-4.25 (m, 2H), 4.18-3.92 | addition of INT |
| | | (m, 9H), 3.91-3.80 (m, 3H), 3.79-3.67 (m, 4H), 3.63- | 26-1, the |
| | | 3.56 (m, 1H), 3.12-3.00 (m, 2H), 2.87-2.69 (m, 2H), | reaction mixture |
| | | 2.56-1.80 (m, 23H), 1.60-1.49 (m, 1H), 1.42-1.29 | was stirred at |
| | | (m, 1H), 1.26-1.16 (m, 3H). | 55° C. |
| | | | overnight. |
| 2-28 | 1043.5 | δ 8.20-8.10 (m, 2H), 7.76-7.64 (m, 2H), 7.47 (t, J = | INT 29-1 |
| | | 8.9 Hz, 1H), 6.59-6.49 (m, 2H), 4.79-4.64 (m, 2H), | in step 1 |
| | | 4.60-4.46 (m, 2H), 4.16-3.81 (m, 9H), 3.77-3.66 | |
| | | (m, 4H), 3.63-3.54 (m, 1H), 3.45-3.40 (m, 1H), | |
| | | 3.23-3.07 (m, 2H), 2.84-2.66 (m, 6H), 2.46-2.05 (m, | |
| | | 14H), 1.78-1.51 (m, 6H), 1.29-1.00 (m, 7H). | |
| 2-29 | 1149.0 | δ 8.24-8.07 (m, 2H), 7.99-7.88 (m, 1H), 7.83-7.62 | INT 24-1 |
| | | (m, 3H), 7.53-7.41 (m, 1H), 7.40-7.33 (m, 1H), 7.22- | in step 1 |
| | | 7.07 (m, 3H), 4.81-4.65 (m, 2H), 4.64-4.49 (m, 2H), | |
| | | 4.48-4.23 (m, 4H), 4.18-3.82 (m, 10H), 3.79-3.68 | |
| | | (m, 3H), 3.67-3.42 (m, 7H), 3.25-2.95 (m, 3H), | |
| | | 2.91-2.65 (m, 6H), 2.59-1.92 (m, 17H), 1.69-1.47 (m, | |
| | | 4H). | |
| 2-30 | 1062.5 | δ 8.24-8.12 (m, 2H), 7.86-7.65 (m, 3H), 7.62-7.41 | INT 19-1 |
| | | (m, 3H), 5.18 (dd, J = 13.3, 5.1 Hz, 1H), 4.83-4.63 (m, | in step 1 |
| | | 2H), 4.63-4.43 (m, 4H), 4.41-4.17 (m, 4H), 4.17- | |
| | | 3.82 (m, 6H), 3.80-3.67 (m, 4H), 3.65-3.56 (m, 1H), | |
| | | 3.48-3.40 (m, 2H), 3.24-3.02 (m, 7H), 2.99-2.76 | |
| | | (m, 4H), 2.60-1.93 (m, 19H), 1.92-1.73 (m, 2H). | |
| 2-31 | 1075.6 | δ 8.21-8.08 (m, 2H), 7.94 (d, J = 8.8 Hz, 1H), 7.82- | INT 36-1 |
| | | 7.61 (m, 3H), 7.53-7.43 (m, 1H), 7.38-7.31 (m, 1H), | in step 1 |
| | | 4.82-4.74 (m, 1H), 4.74-4.65 (m, 1H), 4.63-4.54 | |
| | | (m, 1H), 4.52-4.39 (m, 3H), 4.38-4.24 (m, 1H), 4.18- | |
| | | 3.92 (m, 8H), 3.92-3.83 (m, 1H), 3.82-3.68 (m, 4H), | |
| | | 3.68-3.55 (m, 3H), 3.49-3.39 (m, 2H), 3.13-3.06 | |
| | | (m, 1H), 3.06-2.96 (m, 1H), 2.89-2.72 (m, 2H), | |
| | | 2.59-1.96 (m, 17H), 1.95-1.75 (m, 3H), 1.75-1.58 | |
| | | (m, 3H), 1.40-1.00 (m, 7H). | |
| 2-32 | 1075.6 | δ 8.19-8.05 (m, 2H), 7.95-7.86 (m, 1H), 7.75-7.58 | INT 36-2 |
| | | (m, 3H), 7.48-7.38 (m, 1H), 7.31-7.21 (m, 1H), 4.80- | in step 1 |
| | | 4.74 (m, 1H), 4.73-4.65 (m, 1H), 4.63-4.53 (m, 1H), | |
| | | 4.49-4.40 (m, 3H), 4.38-4.27 (m, 1H), 4.17-3.92 | |
| | | (m, 8H), 3.92-3.83 (m, 1H), 3.82-3.67 (m, 5H), 3.65- | |
| | | 3.55 (m, 2H), 3.49-3.39 (m, 2H), 3.13-3.05 (m, 1H), | |
| | | 2.90-2.72 (m, 2H), 2.58-1.90 (m, 19H), 1.88-1.75 | |
| | | (m, 2H), 1.75-1.53 (m, 3H), 1.37 (t, J = 7.1 Hz, | |
| | | 2H), 1.32-1.13 (m, 5H). | |
| 2-33 | 1075.6 | δ 8.21-8.09 (m, 2H), 7.93-7.85 (m, 1H), 7.76-7.55 | INT 36-3 |
| | | (m, 3H), 7.52-7.43 (m, 1H), 7.33-7.25 (m, 1H), 4.81- | in step 1 |
| | | 4.75 (m, 1H), 4.71-4.63 (m, 1H), 4.62-4.45 (m, 3H), | |
| | | 4.45-4.39 (m, 1H), 4.38-4.23 (m, 2H), 4.17-3.67 | |
| | | (m, 13H), 3.65-3.57 (m, 1H), 3.12-2.95 (m, 2H), | |
| | | 2.89-2.70 (m, 2H), 2.57-1.96 (m, 18H), 1.94-1.76 (m, | |
| | | 3H), 1.73-1.54 (m, 4H), 1.40-1.13 (m, 7H), 1.11- | |
| | | 0.99 (m, 1H). | |
| 2-34 | 1075.6 | δ 8.18-8.05 (m, 2H), 7.88 (d, J = 8.8 Hz, 1H), 7.73- | INT 36-4 |
| | | 7.62 (m, 2H), 7.61-7.55 (m, 1H), 7.49-7.37 (m, 1H), | in step 1 |
| | | 7.30-7.23 (m, 1H), 4.78 (d, J = 12.3 Hz, 1H), 4.68 (d, | |
| | | J = 12.8 Hz, 1H), 4.62-4.53 (m, 2H), 4.43 (dd, J = 9.6, | |
| | | 5.1 Hz, 1H), 4.39-4.23 (m, 2H), 4.17-3.82 (m, 10H), | |
| | | 3.81-3.68 (m, 5H), 3.67-3.59 (m, 1H), 3.49-3.42 | |
| | | (m, 2H), 3.12-3.04 (m, 1H), 2.90-2.72 (m, 2H), | |
| | | 2.58-1.88 (m, 19H), 1.87-1.52 (m, 5H), 1.43-1.32 (m, | |
| | | 2H), 1.30-1.14 (m, 5H). | |
| 2-35 | 1062.6 | δ 8.30-8.03 (m, 2H), 7.85-7.63 (m, 3H), 7.48 (t, J = | INT 37-1 |
| | | 8.9 Hz, 1H), 6.72 (dd, J = 9.2, 3.6 Hz, 1H), 4.82-4.67 | in step 1 |
| | | (m, 2H), 4.64-4.42 (m, 4H), 4.30 (s, 2H), 4.20 (dd, J = | |
| | | 9.4, 5.1 Hz, 1H), 4.17-3.90 (m, 6H), 3.86-3.79 (m, | |
| | | 2H), 3.72 (q, J = 13.1, 11.9 Hz, 1H), 3.59 (d, J = 1.0 Hz, | |
| | | 2H), 3.41 (s, 1H), 3.28-3.06 (m, 2H), 2.99-2.64 (m, | |
| | | 7H), 2.43-2.34 (m, 5H), 2.33-1.89 (m, 10H), 1.85- | |
| | | 1.48 (m, 6H), 1.26-0.95 (m, 6H). | |
| 2-36 | 1062.6 | δ 8.85 (d, J = 3.0 Hz, 1H), 8.14 (dd, J = 9.6, 6.3 Hz, 2H), | INT 37-2 |
| | | 7.79-7.62 (m, 2H), 7.47 (t, J = 8.9 Hz, 1H), 7.08 (d, | in step 1 |
| | | J = 3.9 Hz, 1H), 4.80-4.63 (m, 2H), 4.64-4.22 (m, 5H), | |
| | | 4.22-3.81 (m, 12H), 3.81-3.65 (m, 2H), 3.59 (d, J = | |
| | | 4.2 Hz, 1H), 3.42 (s, 1H), 3.27-3.02 (m, 4H), 2.94- | |
| | | 2.69 (m, 4H), 2.64-2.50 (m, 1H), 2.50-2.30 (m, 5H), | |
| | | 2.30-2.02 (m, 7H), 2.02-1.83 (m, 3H), 1.80-1.53 | |
| | | (m, 4H), 1.34 (q, J = 14.0, 13.2 Hz, 2H), 1.22 (d, J = 6.5 | |
| | | Hz, 2H), 1.07 (d, J = 12.5 Hz, 2H). | |
| 2-37 | 1063.6 | δ 8.85 (d, J = 0.8 Hz, 1H), 8.25-8.07 (m, 2H), 7.80- | INT 37-3 |
| | | 7.65 (m, 2H), 7.49 (t, J = 8.9 Hz, 1H), 4.86-4.65 (m, | in step 1 |
| | | 4H), 4.64-4.43 (m, 2H), 4.29 (dd, J = 9.8, 5.1 Hz, 3H), | |
| | | 4.20-3.80 (m, 10H), 3.73 (qd, J = 11.7, 2.8 Hz, 1H), | |
| | | 3.67-3.54 (m, 2H), 3.41 (t, J = 9.0 Hz, 1H), 3.28-3.09 | |
| | | (m, 2H), 3.02 (t, J = 12.6 Hz, 2H), 2.89-2.66 (m, 4H), | |
| | | 2.59-2.30 (m, 6H), 2.30-1.95 (m, 8H), 1.89-1.50 | |
| | | (m, 6H), 1.26-0.96 (m, 6H). | |
| 2-38 | 1063.6 | δ 8.22 (d, J = 4.3 Hz, 1H), 8.20-8.06 (m, 2H), 7.78- | INT 37-4 |
| | | 7.64 (m, 2H), 7.47 (t, J = 8.9 Hz, 1H), 4.82-4.66 (m, | in step 1 |
| | | 2H), 4.65-4.43 (m, 4H), 4.42-4.21 (m, 3H), 4.22- | |
| | | 3.78 (m, 11H), 3.78-3.66 (m, 1H), 3.66-3.58 (m, 1H), | |
| | | 3.42 (s, 1H), 3.28-3.06 (m, 2H), 2.98 (t, J = 12.7 Hz, | |
| | | 2H), 2.88-2.65 (m, 4H), 2.62-1.96 (m, 14H), 1.89- | |
| | | 1.47 (m, 6H), 1.25-0.97 (m, 6H). | |
| 2-39 | 1075.6 | δ 8.15 (t, J = 8.7 Hz, 2H), 7.95 (d, J = 8.8 Hz, 1H), 7.82 | INT 14-5 |
| | | (s, 1H), 7.71 (dt, J = 12.5, 7.2 Hz, 2H), 7.47 (t, J = 8.9 | in step 1 |
| | | Hz, 1H), 7.36 (d, J = 8.9 Hz, 1H), 4.80-4.63 (m, 2H), | |
| | | 4.63-4.22 (m, 6H), 4.22-3.82 (m, 10H), 3.81-3.54 | |
| | | (m, 6H), 3.43 (s, 1H), 3.27-3.03 (m, 2H), 2.92-2.68 | |
| | | (m, 4H), 2.60-1.96 (m, 16H), 1.84-1.58 (m, 5H), | |
| | | 1.51-1.27 (m, 5H), 1.09 (q, J = 11.9, 11.2 Hz, 2H). | |
| 2-40 | 1089.6 | δ 8.23-8.10 (m, 2H), 7.96 (dd, J = 8.8, 3.0 Hz, 1H), | INT 14-6 |
| | | 7.88 (d, J = 1.9 Hz, 1H), 7.72 (tt, J = 9.9, 6.9 Hz, 2H), | in step 1 |
| | | 7.48 (dd, J = 9.9, 7.8 Hz, 1H), 7.42-7.32 (m, 1H), | |
| | | 4.83-4.65 (m, 2H), 4.66-4.22 (m, 5H), 4.20-3.83 (m, | |
| | | 10H), 3.80-3.57 (m, 6H), 3.44 (q, J = 7.2 Hz, 1H), | |
| | | 3.28-3.06 (m, 2H), 2.89-2.69 (m, 4H), 2.58-1.95 (m, | |
| | | 16H), 1.73 (d, J = 16.7 Hz, 5H), 1.53-1.19 (m, 8H), | |
| | | 1.09 (dd, J = 19.4, 9.6 Hz, 2H). | |
| 2-41 | 1079.6 | δ 8.16 (q, J = 10.8, 9.3 Hz, 2H), 7.72 (p, J = 8.0, 7.5 Hz, | INT 31-3 |
| | | 2H), 7.54-7.41 (m, 2H), 7.20 (d, J = 6.6 Hz, 1H), 4.73 | in step 1 |
| | | (d, J = 13.6 Hz, 2H), 4.64-4.21 (m, 5H), 4.21-3.66 | |
| | | (m, 12H), 3.57 (d, J = 14.9 Hz, 3H), 3.45 (d, J = 21.9 | |
| | | Hz, 1H), 3.27-3.05 (m, 2H), 2.97-2.67 (m, 6H), 2.53- | |
| | | 1.95 (m, 14H), 1.87 (d, J = 12.7 Hz, 2H), 1.78-1.54 | |
| | | (m, 4H), 1.47 (q, J = 10.6, 9.9 Hz, 2H), 1.24 (s, 2H), 1.08 | |
| | | (q, J = 12.0, 11.3 Hz, 2H). | |
| 2-42 | 1075.6 | δ 8.21-8.08 (m, 2H), 7.94 (dd, J = 8.9, 1.8 Hz, 1H), | INT 35-1B |
| | | 7.81 (d, J = 2.0 Hz, 1H), 7.77-7.65 (m, 2H), 7.47 (td, | in step 1 |
| | | J = 9.0, 3.6 Hz, 1H), 7.35 (dt, J = 9.1, 2.6 Hz, 1H), 4.80- | |
| | | 4.65 (m, 2H), 4.65-4.20 (m, 5H), 4.18-3.81 (m, 10H), | |
| | | 3.81-3.55 (m, 6H), 3.48-3.37 (m, 2H), 3.27-2.94 | |
| | | (m, 3H), 2.94-2.68 (m, 3H), 2.59-2.05 (m, 15H), 2.02- | |
| | | 1.95 (m, 1H), 1.90-1.60 (m, 5H), 1.53-1.17 (m, 4H), | |
| | | 0.87 (d, J = 6.9 Hz, 3H). | |
| 2-43 | 1079.6 | δ 8.15 (dtd, J = 9.5, 6.6, 5.6, 3.9 Hz, 2H), 7.93 (d, J = | INT 34-1B |
| | | 8.8 Hz, 1H), 7.80 (d, J = 2.1 Hz, 1H), 7.76-7.65 (m, | in step 1 |
| | | 2H), 7.47 (td, J = 8.9, 2.2 Hz, 1H), 7.35 (dt, J = 8.9, 2.2 | |
| | | Hz, 1H), 4.74-4.62 (m, 3H), 4.58 (dd, J = 14.4, 6.2 Hz, | |
| | | 2H), 4.51-4.09 (m, 7H), 4.06-3.81 (m, 4H), 3.81- | |
| | | 3.69 (m, 4H), 3.69-3.55 (m, 3H), 3.55-3.36 (m, 2H), | |
| | | 3.28-2.98 (m, 3H), 2.98-2.68 (m, 3H), 2.59-2.29 | |
| | | (m, 6H), 2.29-2.05 (m, 9H), 2.02-1.81 (m, 3H), 1.70 | |
| | | (d, J = 13.5 Hz, 3H), 1.61-1.31 (m, 4H). | |
| 2-44 | 1079.6 | δ 8.14 (dd, J = 10.6, 6.8 Hz, 2H), 7.93 (d, J = 8.8 Hz, | INT 34-1A |
| | | 1H), 7.83-7.65 (m, 3H), 7.47 (t, J = 8.9 Hz, 1H), 7.35 | in step 1 |
| | | (dd, J = 8.9, 2.0 Hz, 1H), 4.80-4.66 (m, 2H), 4.67- | |
| | | 4.12 (m, 8H), 4.12-3.81 (m, 8H), 3.81-3.70 (m, 3H), | |
| | | 3.64 (t, J = 12.1 Hz, 3H), 3.50-3.35 (m, 1H), 3.27- | |
| | | 2.66 (m, 6H), 2.58-2.29 (m, 6H), 2.29-2.04 (m, 9H), | |
| | | 1.99 (t, J = 7.5 Hz, 1H), 1.92-1.80 (m, 2H), 1.79-1.61 | |
| | | (m, 3H), 1.62-1.45 (m, 3H), 1.44-1.32 (m, 1H). | |
| 2-45 | 1079.6 | δ 8.13 (dd, J = 9.5, 6.1 Hz, 2H), 7.92 (d, J = 8.8 Hz, 1H), | INT 34-1C |
| | | 7.80-7.65 (m, 3H), 7.47 (t, J = 8.9 Hz, 1H), 7.33 (dd, | in step 1 |
| | | J = 8.8, 2.1 Hz, 1H), 4.80-4.63 (m, 2H), 4.62-4.22 (m, | |
| | | 6H), 4.21-3.69 (m, 13H), 3.61 (s, 3H), 3.54-3.35 (m, | |
| | | 2H), 3.27-2.88 (m, 4H), 2.88-2.68 (m, 2H), 2.57- | |
| | | 2.05 (m, 14H), 2.02-1.55 (m, 7H), 1.40-1.18 (m, 3H). | |
| 2-46 | 1075.6 | δ 8.26-8.08 (m, 2H), 7.95 (dd, J = 8.8, 1.8 Hz, 1H), | INT 38-1 |
| | | 7.88 (d, J = 2.4 Hz, 1H), 7.80-7.63 (m, 2H), 7.48 (td, | in step 1 |
| | | J = 9.0, 1.9 Hz, 1H), 7.37 (dt, J = 8.5, 2.4 Hz, 1H), 4.83- | |
| | | 4.65 (m, 2H), 4.66-4.24 (m, 7H), 4.24-3.82 (m, 7H), | |
| | | 3.82-3.53 (m, 7H), 3.42 (s, 1H), 3.28-3.04 (m, 2H), | |
| | | 2.91-2.68 (m, 4H), 2.62-1.83 (m, 17H), 1.81-1.53 | |
| | | (m, 5H), 1.48 (td, J = 7.2, 1.9 Hz, 3H), 1.30 (d, J = 7.0 | |
| | | Hz, 2H), 1.10 (q, J = 11.7 Hz, 2H). | |
| 2-47 | 1089.6 | δ 8.23-8.10 (m, 2H), 8.00-7.86 (m, 2H), 7.77-7.65 | INT 38-2 |
| | | (m, 2H), 7.47 (td, J = 9.0, 2.3 Hz, 1H), 7.42-7.28 (m, | in step 1 |
| | | 1H), 5.01-4.91 (m, 1H), 4.83-4.65 (m, 2H), 4.65- | |
| | | 4.24 (m, 5H), 4.22-3.57 (m, 14H), 3.49-3.36 (m, 2H), | |
| | | 3.27-3.04 (m, 2H), 2.94-2.68 (m, 4H), 2.60-1.83 | |
| | | (m, 17H), 1.83-1.49 (m, 10H), 1.30 (d, J = 7.2 Hz, 2H), | |
| | | 1.10 (q, J = 11.3 Hz, 2H). | |
| 2-48 | 1062.6 | δ 8.19-8.13 (m, 2H), 7.75-7.65 (m, 2H), 7.63 (d, J = | INT 14-7 |
| | | 8.9 Hz, 1H), 7.47 (t, J = 8.9 Hz, 1H), 6.99 (dd, J = 8.9, | in step 1 |
| | | 2.0 Hz, 1H), 6.93 (d, J = 2.0 Hz, 1H), 4.80-4.62 (m, | |
| | | 2H), 4.62-3.53 (m, 21H), 3.43 (s, 2H), 3.27-3.01 (m, | |
| | | 7H), 2.99-2.65 (m, 4H), 2.52-1.91 (m, 16H), 1.90- | |
| | | 1.72 (m, 2H), 1.25 (dd, J = 14.0, 10.2 Hz, 2H). | |
| 2-49 | 1061.5 | δ 8.23-8.09 (m, 2H), 7.79-7.63 (m, 3H), 7.47 (t, J = | INT 21-2 |
| | | 8.9 Hz, 1H), 7.37 (s, 1H), 7.10 (d, J = 8.5 Hz, 1H), | in step 1 |
| | | 4.81-4.64 (m, 2H), 4.41-3.80 (m, 2H), 4.43-3.81 (m, | |
| | | 13H), 3.81-3.66 (m, 3H), 3.60 (s, 1H), 3.49-3.37 (m, | |
| | | 2H), 3.27-3.00 (m, 7H), 3.00-2.68 (m, 4H), 2.55- | |
| | | 1.95 (m, 18H), 1.82 (s, 2H), 1.38-1.18 (m, 3H). | |
| 2-50 | 1097.6 | δ 8.25-8.06 (m, 2H), 8.01-7.85 (m, 1H), 7.80-7.64 | INT 38-3 |
| | | (m, 3H), 7.53-7.37 (m, 2H), 4.83-4.67 (m, 2H), | in step 1 |
| | | 4.66-4.23 (m, 5H), 4.23-3.66 (m, 11H), 3.48-3.33 (m, | |
| | | 4H), 3.27-3.06 (m, 2H), 2.95-2.67 (m, 4H), 2.63- | |
| | | 2.32 (m, 6H), 2.29-2.04 (m, 8H), 2.05-1.89 (m, 3H), | |
| | | 1.89-1.43 (m, 6H), 1.25 (s, 2H), 1.09 (q, J = 11.7 Hz, | |
| | | J = 11.3 Hz, 2H). | |
| 2-51 | 1079.6 | δ 8.28-8.04 (m, 2H), 7.80-7.64 (m, 2H), 7.57 (d, J = | INT 39-1A |
| | | 9.0 Hz, 1H), 7.47 (t, J = 8.9 Hz, 1H), 6.98 (dd, J = 9.1, | in step 1 |
| | | 2.0 Hz, 1H), 6.90 (s, 1H), 4.80-4.65 (m, 2H), 4.64- | |
| | | 4.21 (m, 6H), 4.20-3.84 (m, 11H), 3.78-3.52 (m, 4H), | |
| | | 3.48-3.36 (m, 1H), 3.27-3.05 (m, 2H), 2.94-2.61 | |
| | | (m, 6H), 2.58-1.90 (m, 15H), 1.88-1.53 (m, 5H), 1.40 | |
| | | (q, J = 12.0, 11.5 Hz, 1H), 1.27-0.98 (m, 3H). | |
| 2-52 | 1079.6 | δ 8.15 (dd, J = 13.0, 7.3 Hz, 2H), 7.79-7.65 (m, 2H), | INT 39-1B |
| | | 7.57 (d, J = 8.9 Hz, 1H), 7.47 (t, J = 8.9 Hz, 1H), 6.98 | in step 1 |
| | | (dt, J = 9.1, 1.9 Hz, 1H), 6.90 (d, J = 2.3 Hz, 1H), | |
| | | 4.81-4.64 (m, 2H), 4.64-4.22 (m, 6H), 4.22-3.53 (m, | |
| | | 15H), 3.42 (s, 1H), 3.28-3.05 (m, 2H), 2.92-2.63 (m, | |
| | | 6H), 2.56-1.90 (m, 15H), 1.83-1.54 (m, 5H), 1.41 (q, | |
| | | J = 11.3 Hz, 1H), 1.28-0.95 (m, 3H). | |
| 2-53 | 1079.6 | δ 8.23-8.09 (m, 2H), 7.81-7.63 (m, 3H), 7.48 (t, J = | INT 40-1A |
| | | 8.9 Hz, 1H), 7.21-7.03 (m, 2H), 4.81-4.65 (m, 2H), | in step 1 |
| | | 4.61-4.22 (m, 5H), 4.22-3.67 (m, 14H), 3.59 (dd, J = | |
| | | 8.9, 2.7 Hz, 1H), 3.41 (s, 1H), 3.28-3.06 (m, 4H), | |
| | | 2.92-2.65 (m, 4H), 2.56-1.53 (m, 21H), 1.46 (dd, J = 13.3, | |
| | | 6.5 Hz, 1H), 1.32 (dd, J = 13.8, 7.0 Hz, 1H), 1.10 (q, J = | |
| | | 12.8, 12.3 Hz, 2H). | |
| 2-54 | 1079.6 | δ 8.24-8.08 (m, 2H), 7.80-7.63 (m, 3H), 7.48 (t, J = | INT 40-1B |
| | | 8.9 Hz, 1H), 7.18-7.03 (m, 2H), 4.74 (qd, J = 12.3, 5.4 | in step 1 |
| | | Hz, 3H), 4.64-4.23 (m, 5H), 4.23-3.66 (m, 14H), 3.60 | |
| | | (d, J = 4.6 Hz, 1H), 3.42 (s, 1H), 3.29-3.07 (m, 4H), | |
| | | 2.87-2.65 (m, 4H), 2.53-1.95 (m, 14H), 1.92-1.54 | |
| | | (m, 6H), 1.45 (d, J = 6.7 Hz, 1H), 1.39-1.25 (m, 1H), | |
| | | 1.09 (d, J = 11.4 Hz, 2H). | |
| 2-55 | 1075.6 | δ 8.22-8.10 (m, 2H), 7.94 (dd, J = 8.9, 1.5 Hz, 1H), | INT 41-1A |
| | | 7.81 (t, J = 2.5 Hz, 1H), 7.71 (pd, J = 7.3, 1.9 Hz, 2H), | in step 1 |
| | | 7.47 (td, J = 9.0, 2.0 Hz, 1H), 7.36 (dt, J = 9.0, 2.3 Hz, | |
| | | 1H), 4.75 (dt, J = 22.9, 12.2 Hz, 2H), 4.66-4.25 (m, | |
| | | 5H), 4.25-3.81 (m, 10H), 3.81-3.53 (m, 6H), 3.51- | |
| | | 3.33 (m, 2H), 3.28-3.04 (m, 2H), 2.94-2.68 (m, 4H), | |
| | | 2.59-1.91 (m, 15H), 1.91-1.39 (m, 7H), 1.26-0.92 | |
| | | (m, 6H). | |
| 2-56 | 1076.6 | δ 8.15 (t, J = 9.5 Hz, 2H), 7.94 (dd, J = 9.0, 1.8 Hz, 1H), | INT 41-1B |
| | | 7.81 (s, 1H), 7.77-7.64 (m, 2H), 7.48 (t, J = 8.9 Hz, | in step 1 |
| | | 1H), 7.36 (d, J = 8.9 Hz, 1H), 4.75 (q, J = 12.1 Hz, 2H), | |
| | | 4.66-4.24 (m, 5H), 4.24-3.82 (m, 10H), 3.82-3.51 | |
| | | (m, 6H), 3.51-3.33 (m, 2H), 3.28-3.04 (m, 2H), 2.92- | |
| | | 2.68 (m, 4H), 2.59-2.05 (m, 14H), 1.99 (d, J = 11.3 | |
| | | Hz, 1H), 1.83 (s, 2H), 1.75-1.39 (m, 5H), 1.27-0.94 | |
| | | (m, 6H). | |
| 2-57 | 1097.6 | δ 8.25-8.06 (m, 2H), 7.80-7.63 (m, 2H), 7.60-7.39 | INT 42-1A |
| | | (m, 2H), 6.94 (dd, J = 8.7, 2.0 Hz, 1H), 6.83 (d, J = 2.0 | in step 1 |
| | | Hz, 1H), 4.74 (qd, J = 12.4, 5.4 Hz, 2H), 4.56 (dt, J = | |
| | | 22.7, 11.3 Hz, 2H), 4.29 (dd, J = 9.2, 5.1 Hz, 3H), | |
| | | 4.22-3.68 (m, 13H), 3.67-3.55 (m, 1H), 3.42 (s, 1H), | |
| | | 3.28-2.94 (m, 3H), 2.94-2.63 (m, 5H), 2.58-1.87 (m, | |
| | | 16H), 1.84-1.50 (m, 5H), 1.35-0.96 (m, 4H). | |
| 2-58 | 1097.6 | δ 8.25-8.06 (m, 2H), 7.80-7.64 (m, 2H), 7.56 (d, J = | INT 42-1B |
| | | 8.9 Hz, 1H), 7.47 (t, J = 8.9 Hz, 1H), 6.94 (dt, J = 9.1, | in step 1 |
| | | 2.1 Hz, 1H), 6.83 (d, J = 2.4 Hz, 1H), 4.81-4.66 (m, | |
| | | 2H), 4.65-4.22 (m, 5H), 4.21-3.67 (m, 13H), 3.59 (d, | |
| | | J = 4.8 Hz, 1H), 3.42 (s, 1H), 3.27-2.95 (m, 3H), | |
| | | 2.93-2.65 (m, 6H), 2.54-1.88 (m, 16H), 1.81-1.54 (m, | |
| | | 5H), 1.36-0.94 (m, 3H). | |
| 2-59 | 1097.6 | δ 8.24-8.06 (m, 2H), 7.93 (d, J = 8.9 Hz, 1H), 7.84- | INT 42-1C |
| | | 7.63 (m, 3H), 7.48 (t, J = 8.9 Hz, 1H), 7.34 (dd, J = 8.9, | in step 1 |
| | | 1.9 Hz, 1H), 4.82-4.66 (m, 2H), 4.66-4.24 (m, 6H), | |
| | | 4.24-3.57 (m, 16H), 3.43 (s, 1H), 3.26-3.05 (m, 3H), | |
| | | 2.96 (d, J = 13.1 Hz, 1H), 2.88-2.68 (m, 2H), 2.58- | |
| | | 2.04 (m, 16H), 2.02-1.82 (m, 3H), 1.80-1.55 (m, 3H), | |
| | | 1.55-1.24 (m, 2H). | |
| 2-60 | 1097.6 | δ 8.16 (tdd, J = 8.9, 7.0, 3.7 Hz, 2H), 7.94 (d, J = 8.8 Hz, | INT 42-1D |
| | | 1H), 7.80 (d, J = 1.9 Hz, 1H), 7.71 (dq, J = 9.8, 7.5, 6.6 | in step 1 |
| | | Hz, 2H), 7.48 (td, J = 9.0, 1.4 Hz, 1H), 7.35 (dd, J = 8.8, | |
| | | 1.9 Hz, 1H), 4.74 (d, J = 12.6 Hz, 2H), 4.64-4.22 (m, | |
| | | 6H), 4.22-3.52 (m, 16H), 3.51-3.35 (m, 1H), 3.26- | |
| | | 3.04 (m, 3H), 2.97 (t, J = 13.0 Hz, 1H), 2.89-2.67 (m, | |
| | | 2H), 2.60-2.05 (m, 16H), 2.02-1.83 (m, 3H), 1.81- | |
| | | 1.56 (m, 3H), 1.51-1.25 (m, 2H). | |
| 2-61 | 1075.6 | δ 8.16 (dddd, J = 10.7, 9.1, 6.2, 2.2 Hz, 2H), 7.97 (dd, | INT 36-5 |
| | | J = 8.8, 3.7 Hz, 1H), 7.87 (s, 1H), 7.72 (pd, J = 7.3, 2.4 | in step 1 |
| | | Hz, 2H), 7.47 (td, J = 8.9, 4.3 Hz, 1H), 7.39-7.31 (m, | |
| | | 1H), 4.84-4.65 (m, 2H), 4.66-4.24 (m, 5H), 4.24- | |
| | | 3.81 (m, 11H), 3.77-3.55 (m, 5H), 3.49-3.36 (m, 1H), | |
| | | 3.28-3.05 (m, 2H), 2.91-2.68 (m, 4H), 2.58-1.96 | |
| | | (m, 18H), 1.87-1.67 (m, 3H), 1.63-1.46 (m, 3H), | |
| | | 1.23-1.01 (m, 5H). | |
| 2-62 | 1075.6 | δ 8.16 (tdd, J = 10.2, 5.9, 3.3 Hz, 1H), 8.01 (dd, J = 8.8, | INT 36-6 |
| | | 2.6 Hz, 1H), 7.89 (s, 1H), 7.79-7.65 (m, 1H), 7.48 (t, | in step 1 |
| | | J = 8.9 Hz, 1H), 7.37 (s, 1H), 8 4.74 (qd, J = 12.2, 5.7 Hz, | |
| | | 2H), 4.64-4.23 (m, 5H), 4.23-3.57 (m, 17H), 3.42 (s, | |
| | | 1H), 3.29-3.04 (m, 2H), 2.94-2.69 (m, 4H), 2.62- | |
| | | 1.92 (m, 18H), 1.82-1.47 (m, 5H), 1.30 (d, J = 6.6 Hz, | |
| | | 2H), 1.09 (dd, J = 6.4, 2.7 Hz, 5H). | |
| 2-63 | 1089.6 | δ 8.25-8.05 (m, 2H), 7.94 (dd, J = 8.8, 5.3 Hz, 1H), | INT 42-1 |
| | | 7.90-7.77 (m, 1H), 7.77-7.63 (m, 2H), 7.46 (q, J = | in step 1 |
| | | 9.3 Hz, 1H), 7.40-7.28 (m, 1H), 4.80-4.64 (m, 2H), | |
| | | 4.64-4.17 (m, 7H), 4.15-3.57 (m, 14H), 3.50-3.37 | |
| | | (m, 1H), 3.27-3.03 (m, 2H), 2.89-2.63 (m, 4H), 2.58- | |
| | | 1.96 (m, 15H), 1.90-1.59 (m, 6H), 1.51 (d, J = 11.3 | |
| | | Hz, 1H), 1.32-1.16 (m, 3H), 0.84 (d, J = 3.3 Hz, 6H). | |
| 2-64 | 1087.0 | δ 8.27-7.99 (m, 2H), 7.98-7.82 (m, 1H), 7.80-7.53 | INT 42-2 |
| | | (m, 3H), 7.52-7.39 (m, 1H), 7.34-7.17 (m, 1H), | in step 1 |
| | | 4.82-4.63 (m, 1H), 4.63-4.50 (m, 1H), 4.48-3.66 (m, | |
| | | 18H), 3.65-3.30 (m, 5H), 3.18-3.13 (m, 1H), 3.13- | |
| | | 3.04 (m, 1H), 2.93-2.60 (m, 3H), 2.59-1.80 (m, 14H), | |
| | | 1.80-1.49 (m, 5H), 1.49-1.08 (m, 5H), 0.51-0.30 | |
| | | (m, 5H). | |
| 2-65 | 1079.6 | δ 8.22-8.07 (m, 2H), 7.91 (dd, J = 8.9, 1.7 Hz, 1H), | INT 44-1 |
| | | 7.80-7.64 (m, 3H), 7.47 (td, J = 8.9, 2.3 Hz, 1H), 7.32 | in step 1 |
| | | (dd, J = 8.8, 2.0 Hz, 1H), 4.82-4.65 (m, 2H), 4.64- | |
| | | 4.16 (m, 8H), 4.16-3.67 (m, 12H), 3.67-3.52 (m, 3H), | |
| | | 3.52-3.36 (m, 1H), 3.29-3.06 (m, 2H), 2.96-2.69 | |
| | | (m, 4H), 2.57-1.77 (m, 21H), 1.63 (s, 1H), 1.50-1.22 | |
| | | (m, 2H). | |
| 2-66 | 1075.3 | δ 8.15 (dd, J = 8.8, 6.1 Hz, 2H), 7.91 (d, J = 8.8 Hz, 1H), | INT 45-1 |
| | | 7.77-7.59 (m, 3H), 7.47 (q, J = 8.8 Hz, 1H), 7.30 (d, | in step 1 |
| | | J = 8.8 Hz, 1H), 4.81-3.68 (m, 20H), 3.65-3.40 (m, | |
| | | 1H), 3.24-3.01 (m, 3H), 2.91-2.67 (m, 5H), 2.58- | |
| | | 1.48 (m, 22H), 1.41-1.12 (m, 5H), 0.90 (d, J = 6.9 Hz, | |
| | | 3H). | |
| 2-67 | 1075.3 | δ 8.12 (t, J = 7.5 Hz, 2H), 7.86 (d, J = 8.8 Hz, 1H), 7.72- | INT 45-2 |
| | | 7.63 (m, 2H), 7.57 (s, 1H), 7.46 (t, J = 8.9 Hz, 1H), | in step 1 |
| | | 7.26 (d, J = 9.0 Hz, 1H), 4.79-4.62 (m, 3H), 4.60-4.50 | |
| | | (m, 1H), 4.47-4.27 (m, 2H), 4.23-3.65 (m, 14H), 3.63- | |
| | | 3.55 (m, 1H), 3.51-3.39 (m, 3H), 3.20-3.01 (m, 2H), | |
| | | 2.87-2.68 (m, 4H), 2.56-2.06 (m, 13H), 2.05-1.92 | |
| | | (m, 3H), 1.86-1.54 (m, 6H), 1.39-1.10 (m, 4H), | |
| | | 0.87 (d, J = 6.8 Hz, 3H). | |
| 2-68 | 1096.9 | δ 8.17-8.08 (m, 2H), 7.72-7.61 (m, 3H), 7.46 (t, J = | INT 14-8 |
| | | 8.9 Hz, 1H), 7.07-7.00 (m, 2H), 4.68-4.47 (m, 4H), | in step 1 |
| | | 4.32 (dd, J = 9.3, 5.1 Hz, 2H), 4.12 (dd, J = 14.0, 3.7 Hz, | |
| | | 1H), 4.06-3.93 (m, 5H), 3.93-3.81 (m, 3H), 3.76- | |
| | | 3.66 (m, 3H), 3.62-3.55 (m, 1H), 3.48 (p, J = 1.7 Hz, | |
| | | 1H), 3.13 (p, J = 1.7 Hz, 1H), 2.99-2.91 (m, 2H), | |
| | | 2.84-2.71 (m, 3H), 2.50-2.22 (m, 8H), 2.18-2.03 (m, 7H), | |
| | | 2.00-1.90 (m, 3H), 1.85-1.72 (m, 4H), 1.51-1.39 | |
| | | (m, 2H), 0.96 (d, J = 6.8 Hz, 3H), 0.16 (d, J = 9.1 | |
| | | Hz, 2H). | |
| 2-69 | 1079.6 | δ 8.15 (d, J = 9.7 Hz, 2H), 7.94 (dd, J = 8.9, 2.2 Hz, 1H), | INT 14-9 |
| | | 7.86-7.62 (m, 3H), 7.46 (t, J = 8.8 Hz, 1H), 7.34 (d, | in step 1 |
| | | J = 8.7 Hz, 1H), 4.83-4.67 (m, 2H), 4.65-4.22 (m, 5H), | |
| | | 4.18-3.81 (m, 10H), 3.81-3.51 (m, 7H), 3.51-3.36 | |
| | | (m, 1H), 3.28-3.02 (m, 4H), 2.90-2.68 (m, 2H), | |
| | | 2.60-2.05 (m, 16H), 2.05-1.53 (m, 9H). | |
| 2-70 | 1079.6 | δ 8.23-8.07 (m, 2H), 7.82 (d, J = 8.9 Hz, 1H), 7.78- | INT 14-10 |
| | | 7.65 (m, 2H), 7.56 (d, J = 2.0 Hz, 1H), 7.48 (t, J = 8.9 | in step 1 |
| | | Hz, 1H), 7.25 (dd, J = 8.9, 2.1 Hz, 1H), 4.82-4.66 (m, | |
| | | 2H), 4.66-4.21 (m, 5H), 4.21-3.81 (m, 10H), 3.80- | |
| | | 3.55 (m, 7H), 3.47-3.35 (m, 1H), 3.28-3.06 (m, 2H), | |
| | | 2.93-2.67 (m, 4H), 2.57-1.95 (m, 18H), 1.91-1.56 | |
| | | (m, 5H), 1.18 (q, J = 12.8 Hz, 2H). | |
| 2-71 | 1080.3 | δ 8.62 (s, 1H), 8.23-8.12 (m, 2H), 7.78-7.65 (m, 2H), | INT 31-4 |
| | | 7.49 (t, J = 8.9 Hz, 1H), 4.81-4.66 (m, 2H), 4.60-4.23 | in step 1 |
| | | (m, 5H), 4.16-3.83 (m, 12H), 3.78-3.68 (m, 2H), 3.66- | |
| | | 3.57 (m, 1H), 3.52-3.39 (m, 1H), 3.30-3.16 (m, 1H), | |
| | | 3.15-3.03 (m, 3H), 2.91-2.71 (m, 4H), 2.61-1.93 | |
| | | (m, 14H), 1.88-1.52 (m, 6H), 1.43-1.30 (m, 2H), | |
| | | 1.25-1.19 (m, 2H), 1.15-1.02 (m, 2H). | |
| 2-72 | 1080.3 | δ 8.22-8.11 (m, 2H), 7.79-7.61 (m, 3H), 7.49 (t, J = | INT 31-5 |
| | | 8.9 Hz, 1H), 4.81-4.68 (m, 2H), 4.63-4.44 (m, 2H), | in step 1 |
| | | 4.39-3.85 (m, 15H), 3.80-3.68 (m, 2H), 3.65-3.59 | |
| | | (m, 1H), 3.51-3.36 (m, 1H), 3.28-3.05 (m, 2H), 2.94 | |
| | | (t, J = 12.3 Hz, 2H), 2.87-2.67 (m, 4H), 2.48-1.93 (m, | |
| | | 14H), 1.82-1.59 (m, 6H), 1.41-0.98 (m, 6H). | |
| 2-73 | 1148.9 | δ 8.25-8.04 (m, 2H), 7.99-7.87 (m, 1H), 7.84-7.77 | INT 24-3 |
| | | (m, 1H), 7.76-7.63 (m, 2H), 7.54-7.33 (m, 2H), 7.32- | in step 1 |
| | | 6.95 (m, 3H), 4.82-4.65 (m, 2H), 4.64-4.21 (m, 7H), | |
| | | 4.20-3.58 (m, 15H), 3.56-3.41 (m, 2H), 3.27-3.00 | |
| | | (m, 6H), 2.96-2.60 (m, 5H), 2.58-1.95 (m, 17H), | |
| | | 1.91-1.69 (m, 2H), 1.67-1.51 (m, 2H). | |
| 2-74 | 1148.9 | ) δ 8.19-8.08 (m, 2H), 7.86 (d, J = 8.8 Hz, 1H), 7.76- | INT 24-2 |
| | | 7.60 (m, 2H), 7.59-7.51 (m, 1H), 7.50-7.41 (m, 1H), | in step 1 |
| | | 7.35-7.25 (m, 1H), 7.22-7.16 (m, 1H), 7.15-7.04 | |
| | | (m, 2H), 4.79-4.64 (m, 2H), 4.60-4.26 (m, 5H), | |
| | | 4.21-3.40 (m, 16H), 3.23-2.63 (m, 11H), 2.58-1.90 (m, | |
| | | 17H), 1.88-1.68 (m, 3H), 1.64-1.34 (m, 4H). | |
| 2-75 | 1048.5 | δ 8.21-8.11 (m, 2H), 7.75-7.66 (m, 2H), 7.48 (t, J = | INT 29-2 |
| | | 8.9 Hz, 1H), 6.54 (d, J = 12.3 Hz, 2H), 4.79-4.63 (m, | in step 1 |
| | | 2H), 4.61-4.46 (m, 2H), 4.41-4.32 (m, 1H), 4.27 (s, | |
| | | 1H), 4.16-3.92 (m, 5H), 3.91-3.63 (m, 7H), 3.63- | |
| | | 3.37 (m, 4H), 3.23-3.06 (m, 2H), 3.06-2.95 (m, 2H), | |
| | | 2.49-2.02 (m, 12H), 2.02-1.72 (m, 6H), 1.67-1.39 | |
| | | (m, 5H). | |
| 2-76 | 1060.6 | δ 8.24-8.11 (m, 2H), 7.78-7.68 (m, 2H), 7.62 (dd, J = | INT 49-1 |
| | | 8.4, 3.3 Hz, 1H), 7.50 (t, J = 8.9 Hz, 1H), 7.34-7.26 | in step 1 |
| | | (m, 1H), 7.11-7.02 (m, 1H), 4.80-4.68 (m, 2H), | |
| | | 4.65-4.44 (m, 2H), 4.40-4.25 (m, 3H), 4.20-3.69 (m, | |
| | | 11H), 3.67-3.58 (m, 1H), 3.57-3.38 (m, 2H), 3.28- | |
| | | 3.08 (m, 2H), 2.88-2.58 (m, 5H), 2.56-2.07 (m, 13H), | |
| | | 2.04-1.82 (m, 5H), 1.78-1.42 (m, 6H), 1.26-0.95 | |
| | | (m, 6H). | |
| 2-77 | 1060.6 | δ 8.18-8.08 (m, 2H), 7.75-7.61 (m, 2H), 7.49-7.42 | INT 49-2 |
| | | (m, 1H), 7.36-7.29 (m, 1H), 7.28-7.22 (m, 1H), 7.10 | in step 1 |
| | | (d, J = 8.4 Hz, 1H), 4.78-4.23 (m, 10H), 4.20-3.53 | |
| | | (m, 13H), 3.11-3.02 (m, 1H), 2.90-2.61 (m, 6H), | |
| | | 2.54-2.0 (m, 13H), 1.92-1.50 (m, 11H), 1.21-0.99 (m, | |
| | | 4H) | |
| 2-78 | 1046.6 | δ 8.15 (s, 2H), 7.77-7.67 (m, 2H), 7.63 (d, J = 8.4 Hz, | INT 49-3 |
| | | 1H), 7.48 (t, J = 8.9 Hz, 1H), 7.30 (s, 1H), 7.11-7.02 | in step 1 |
| | | (m, 1H), 4.81-4.65 (m, 2H), 4.64-4.46 (m, 2H), | |
| | | 4.44-4.24 (m, 3H), 4.21-3.82 (m, 10H), 3.81-3.68 (m, | |
| | | 2H), 3.67-3.57 (m, 1H), 3.53-3.39 (m, 2H), 3.24- | |
| | | 3.04 (m, 2H), 2.88-2.55 (m, 5H), 2.53-2.06 (m, 13H), | |
| | | 2.05-1.83 (m, 5H), 1.81-1.65 (m, 2H), 1.60-1.47 | |
| | | (m, 2H), 1.29-1.10 (m, 5H) | |
| 2-79 | 1046.6 | δ 8.19-8.05 (m, 2H), 7.77-7.60 (m, 3H), 7.49-7.39 | INT 49-4 |
| | | (m, 1H), 7.38-7.33 (m, 1H), 7.15-7.08 (m, 1H), 4.80- | in step 1 |
| | | 4.64 (m, 2H), 4.62-4.44 (m, 2H), 4.40-4.22 (m, 3H), | |
| | | 4.21-3.80 (m, 9H), 3.78-3.55 (m, 3H), 3.51-3.39 | |
| | | (m, 2H), 3.25-3.01 (m, 3H), 2.93-2.64 (m, 5H), | |
| | | 2.56-1.94 (m, 13H), 1.92-1.67 (m, 7H), 1.65-1.49 (m, | |
| | | 3H), 1.40-1.27 (m, 2H), 1.12-0.95 (m, 2H). | |
| 2-80 | 1026.5 | δ 8.19-8.00 (m, 2H), 7.74-7.48 (m, 3H), 7.46-7.16 | INT 49-5 |
| | | (m, 2H), 7.13-6.82 (m, 4H), 4.80-4.20 (m, 10H), 4.16- | in step 1 |
| | | 3.79 (m, 8H), 3.77-3.53 (m, 4H), 3.48-3.38 (m, 1H), | |
| | | 3.26-2.99 (m, 3H), 2.86-2.63 (m, 5H), 2.52-1.87 | |
| | | (m, 14H). LCMS: 1026.5. | |
| 2-81 | 1060.6 | δ 8.19-8.04 (m, 2H), 7.74-7.65 (m, 2H 7.63 (d, J = | INT 50-1 |
| | | 8.3 Hz, 1H), 7.47 (t, J = 8.9 Hz, 1H), 7.26 (s, 1H), 7.00 | in step 1 |
| | | (d, J = 8.4 Hz, 1H), 4.79-4.62 (m, 2H), 4.61-4.42 (m, | |
| | | 2H), 4.41-4.20 (m, 3H), 4.18-3.80 (m, 9H), 3.79- | |
| | | 3.38 (m, 5H), 3.26-3.00 (m, 3H), 2.88-2.57 (m, 6H), | |
| | | 2.53-2.04 (m, 12H), 2.02-1.93 (m, 1H), 1.81-1.45 | |
| | | (m, 7H), 1.27-0.79 (m, 8H). | |
| 2-82 | 1060.6 | δ 8.19-8.05 (m, 2H), 7.76-7.58 (m, 3H), 7.47 (t, J = | INT 50-2 |
| | | 8.9 Hz, 1H), 7.26 (s, 1H), 7.02 (d, J = 8.4 Hz, 1H), 4.80- | in step 1 |
| | | 4.64 (m, 2H), 4.62-4.44 (m, 2H), 4.42-4.24 (m, 3H), | |
| | | 4.23-3.81 (m, 9H), 3.80-3.66 (m, 2H), 3.65-3.55 | |
| | | (m, 2H), 3.48-3.38 (m, 2H), 3.27-3.01 (m, 3H), 2.87- | |
| | | 2.64 (m, 5H), 2.54-2.04 (m, 11H), 2.03-1.87 (m, | |
| | | 3H), 1.86-1.65 (m, 2H), 1.62-1.28 (m, 8H), 1.26- | |
| | | 1.01 (m, 3H). | |

### Examples 3-1-3-2

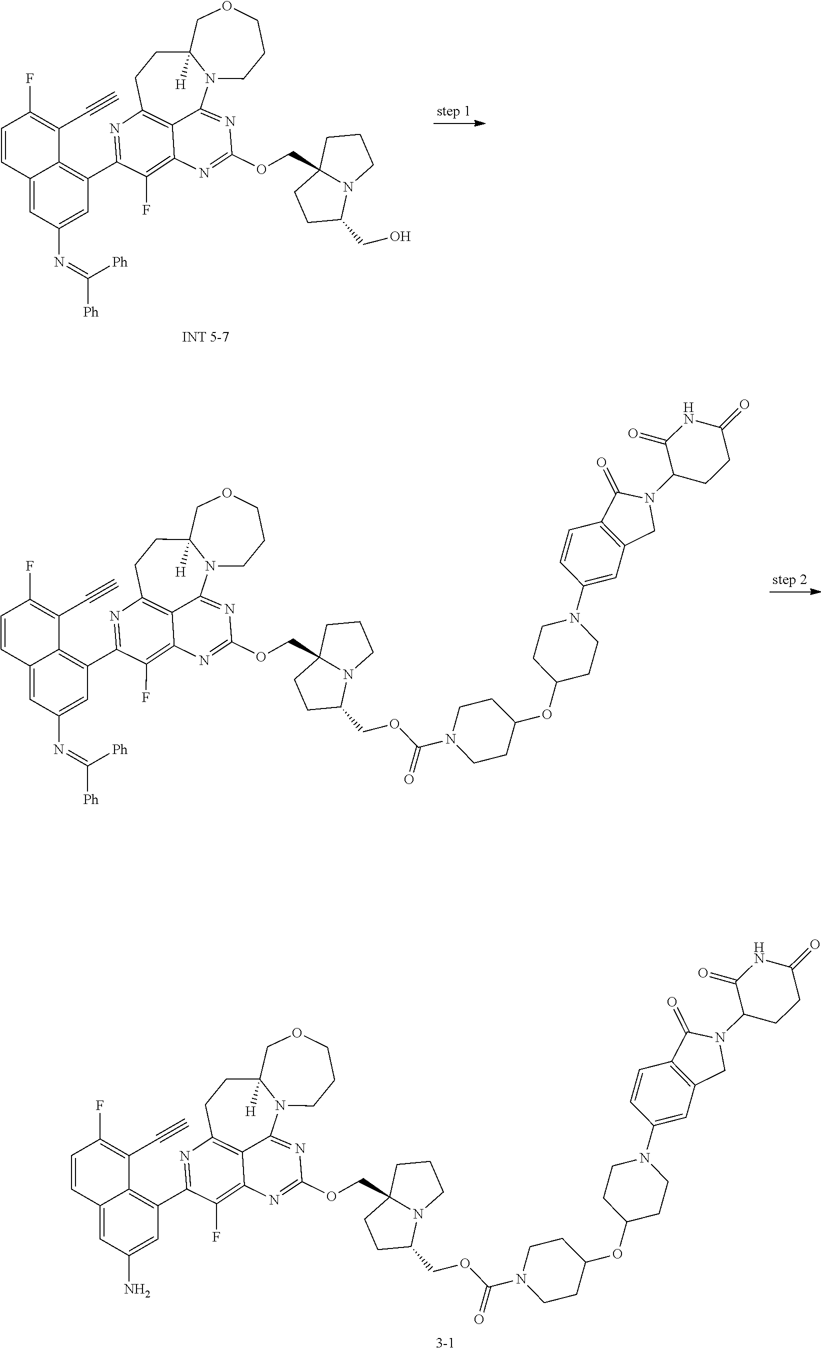

Step 1: ((3S,7aS)-7a-((((S)-2-(3-((diphenylmethylene)amino)-8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(2-(2,6-dioxopiperidin-3-yl)-1-oxoisoindolin-5-yl)piperidin-4-yl)oxy)piperidine-1-carboxylate. To a solution of INT 5-7 (0.04 mmol) in tetrahydrofuran (2.0 mL) at 0° C. was added 4-nitrophenyl chloroformate (0.04 mmol) and diisopropylethylamine (0.11 mmol). The resulting mixture was stirred at 0° C. for 1 hour under an argon atmosphere. To the reaction mixture was added a solution of 3-(1-oxo-5-(4-(piperidin-4-yloxy)piperidin-1-yl)isoindolin-2-yl)piperidine-2,6-dione (INT 9-1) (0.12 mmol) in 0.40 mL 3:1 tetrahydrofuran/dimethyl sulfoxide and diisopropylethylamine (0.04 mmol). The reaction mixture was warmed to room temperature, stirred for 1 hour, then diluted with diethyl ether (10 mL) and the resulting precipitate was used in the following step without further purification.

Step 2: ((3S,7aS)-7a-((((S)-2-(3-amino-8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(2-(2,6-dioxopiperidin-3-yl)-1-oxoisoindolin-5-yl)piperidin-4-yl)oxy)piperidine-1-carboxylate (Example 3-1). To a solution of ((3S,7aS)-7a-((((S)-2-(3-((diphenylmethylene)amino)-8-ethynyl-7-fluoronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(2-(2,6-dioxopiperidin-3-yl)-1-oxoisoindolin-5-yl)piperidin-4-yl)oxy)piperidine-1-carboxylate (0.03 mmol) in acetonitrile (0.8 mL) and dimethyl sulfoxide (0.2 mL) was added hydrochloric acid in 1,4-dioxane (4.0 M, 0.2 mL) and 2 drops of distilled water at room temperature. Upon completion the reaction was concentrated and purified by RP-HPLC (10% to 60% 0.1% TFA in MeCN/0.1% TFA in H₂O). Fractions containing the product were pooled and lyophilized to yield the title compound. ¹H NMR (400 MHz, Methanol-d4) δ 7.91 (dd, J=9.2, 5.7 Hz, 1H), 7.65 (d, J=9.3 Hz, 1H), 7.49 (dd, J=12.8, 2.4 Hz, 1H), 7.43-7.23 (m, 2H), 7.12 (d, J=6.9 Hz, 2H), 5.12 (dd, J=13.3, 5.2 Hz, 1H), 4.77 (d, J=12.3 Hz, 1H), 4.69 (dd, J=12.3, 4.7 Hz, 1H), 4.64-4.47 (m, 2H), 4.45-4.20 (m, 4H), 4.17-3.56 (m, 14H), 3.53-3.38 (m, 2H), 3.29-3.02 (m, 6H), 2.92 (ddd, J=18.4, 13.4, 5.4 Hz, 1H), 2.83-2.74 (m, 1H), 2.56-2.33 (m, 5H), 2.32-2.06 (m, 7H), 2.05-1.92 (m, 3H), 1.83 (s, 2H), 1.72-1.60 (m, 2H), 1.59-1.43 (m, 2H). LCMS: 1079.5.

The following Examples were made in a similar fashion to Example 3-1 and are shown below in Table 3C1. To prepare the below Examples, different reagents/starting materials were used than some of those described in the steps toward Example 3-1 and are noted in the last column of Table 3C2—“Changes to Procedure: Different Reagents/Starting Materials”. A person of ordinary skill in the art will readily recognize which reagents/starting materials used for the synthesis of Example 3-1 were replaced with the different reagents/starting materials noted below.

TABLE 3C1
Compounds

| Example | Structure |
|---|---|
| 3-2 | 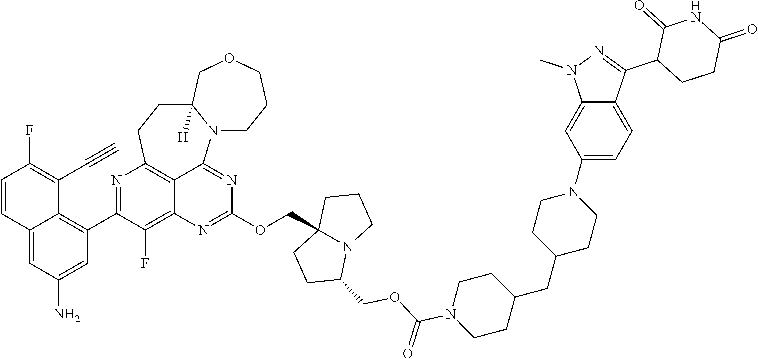 |

TABLE 3C2
Compounds

| | | | Changes |
|---|---|---|---|
| | | | to |
| | | | Procedure: |
| | | | Different |
| | | | Reagents/ |
| | | | Starting |
| Example | LCMS | ¹H-NMR (400 MHz, Methanol-d₄) | Materials |
| 3-2 | 1076.6 | δ 7.92 (d, J = 8.8 Hz, 1H), 7.87-7.80 | INT 14-3 |
| | | (m, 1H), 7.69 (s, 1H), 7.43-7.20 | in step 1 |
| | | (m, 4H), 4.80-4.65 (m, 2H), 4.62-4.24 | |
| | | (m, 6H), 4.21-3.40 (m, 18H), 3.27-3.03 | |
| | | (m, 2H), 2.93-2.71 (m, 4H), 2.58-1.94 | |
| | | (m, 15H), 1.91-1.50 (m, 6H), 1.39-1.24 | |
| | | (m, 2H), 1.20-1.03 (m, 2H). | |

### Examples 4-1-4-9

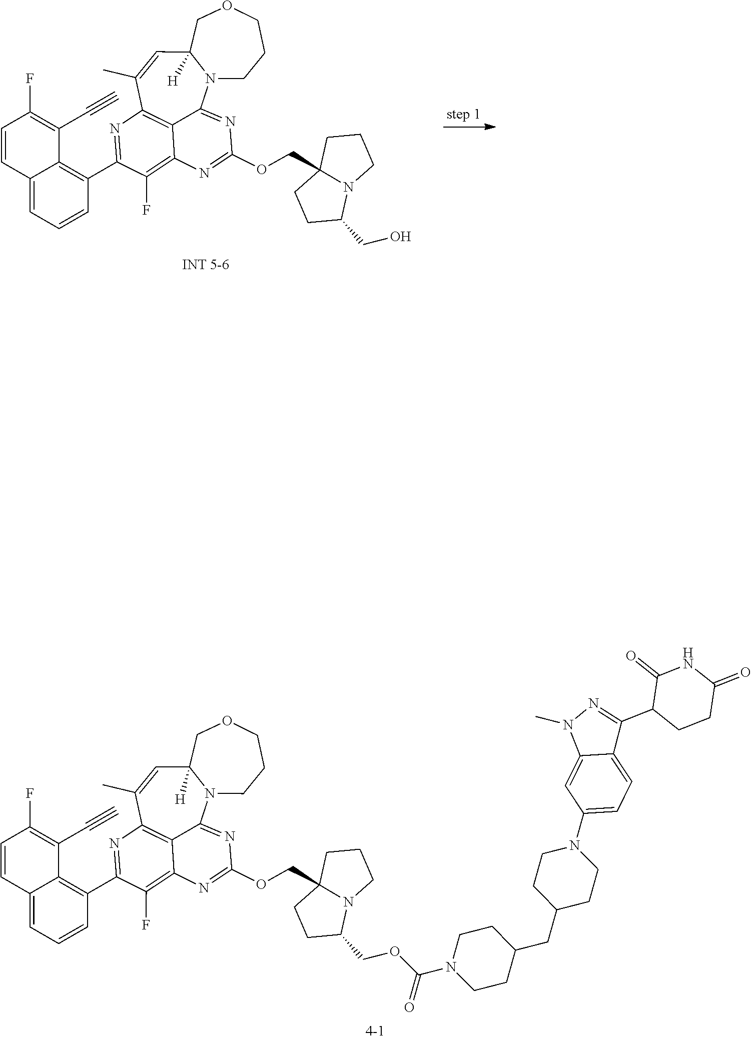

Step 1: ((3S,7aS)-7a-((((S)-2-(8-ethynyl-7-fluoronaphthalen-1-yl)-1-fluoro-4-methyl-5a,6,9,10-tetrahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxopiperidin-3-yl)-1-methyl-1H-indazol-6-yl)piperidin-4-yl)methyl)piperidine-1-carboxylate (Example 4-1). To a solution of INT 5-6 (0.04 mmol) in acetonitrile (1.0 mL) was added carbonyldiimidazole (0.12 mmol). The resulting mixture was stirred at 60° C. for 1 hour under an argon atmosphere. The reaction mixture was diluted with water (10 mL) and dichloromethane (10 mL). The aqueous layer was extracted with dichloromethane (3×10 mL). The organic layers were combined, washed with brine (10 mL), dried over anhydrous sodium sulfate, filtered, and concentrated under reduced pressure. The residue was dissolved in acetonitrile (1 mL) and to the reaction mixture was added 4-dimethylaminopyridine (0.04 mmol). To the reaction mixture was added a solution of INT 14-3 (0.09 mmol) in acetonitrile (1.0 mL), dimethyl sulfoxide (0.25 mL), and diisopropylethylamine (0.2 mL). The reaction mixture was stirred at 70° C. overnight. The reaction mixture was then cooled to room temperature, concentrated, and the resulting residue was purified by RP-HPLC (0 to 50% 0.1% trifluoroacetic acid in acetonitrile/0.1% trifluoroacetic acid in water). Fractions containing the product were pooled and lyophilized to yield the title compound. ¹H NMR (400 MHz, Methanol-d₄) δ 8.20-8.07 (m, 2H), 7.93 (dd, J=8.9, 2.3 Hz, 1H), 7.82-7.60 (m, 3H), 7.46 (td, J=9.0, 2.5 Hz, 1H), 7.39-7.26 (m, 1H), 6.30-6.11 (m, 1H), 4.80-4.60 (m, 3H), 4.59-4.38 (m, 3H), 4.37-4.26 (m, 2H), 4.22-4.10 (m, 5H), 4.09 (d, J=2.6 Hz, 3H), 4.05-3.97 (m, 1H), 3.87-3.73 (m, 3H), 3.67-3.54 (m, 3H), 3.47-3.43 (m, 1H), 2.91-2.70 (m, 4H), 2.59-1.94 (m, 18H), 1.92-1.82 (m, 1H), 1.81-1.53 (m, 5H), 1.36-1.24 (m, 2H), 1.19-1.02 (m, 2H). LCMS: 1073.6.

The following Examples were made in a similar fashion to Example 4-1 and are shown below in Table 3D1. To prepare the below Examples, different reagents/starting materials were used than some of those described in the steps toward Example 4-1 and are noted in the last column of Table 3D2—“Changes to Procedure: Different Reagents/Starting Materials”. A person of ordinary skill in the art will readily recognize which reagents/starting materials used for the synthesis of Example 4-1 were replaced with the different reagents/starting materials noted below.

TABLE 3D1
Compounds

| Example | Structure |
|---|---|
| 4-2 | 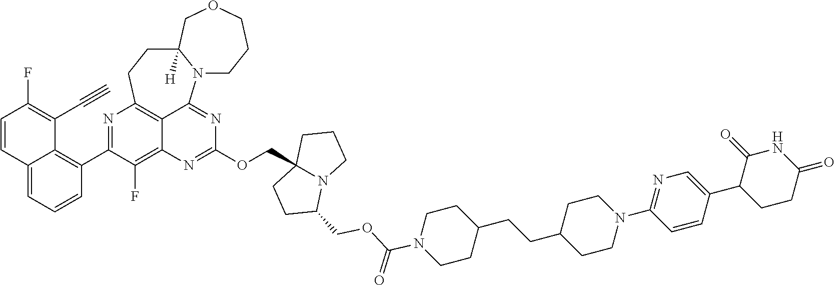 |
| 4-3 | 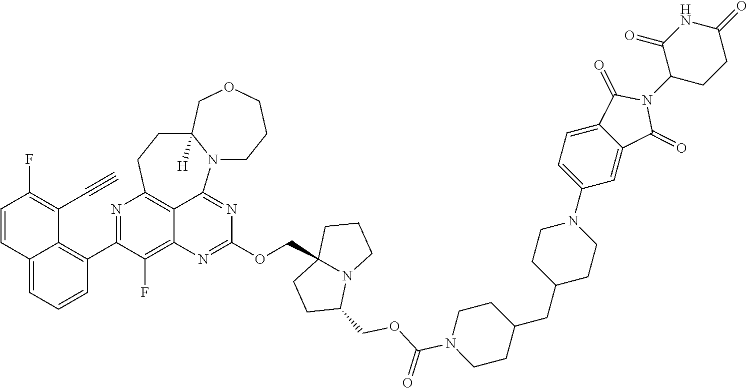 |
| 4-4 | 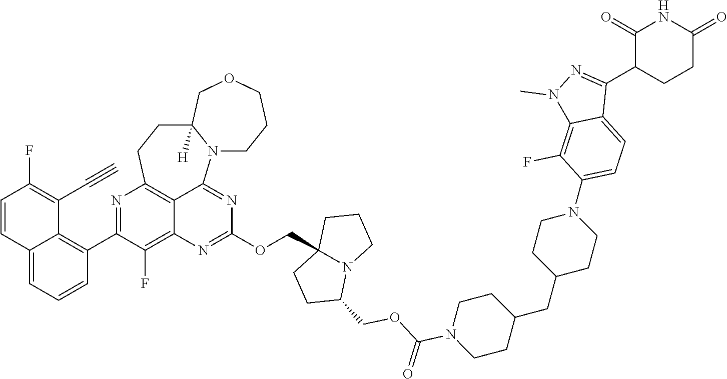 |
| 4-5 | 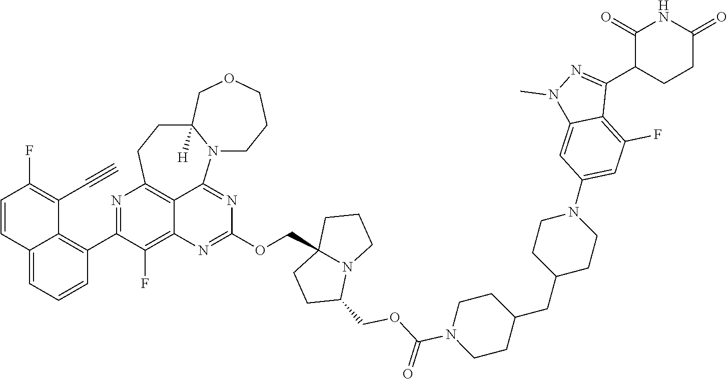 |
| 4-6 | 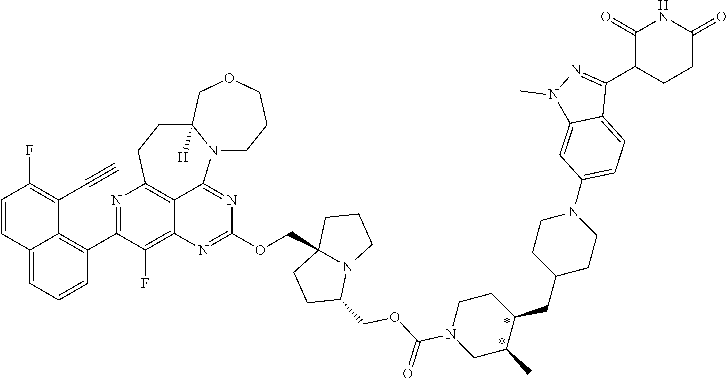 |
| 4-7 | 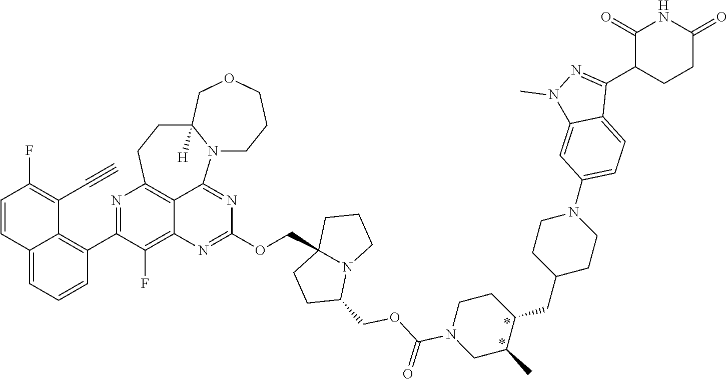 |
| 4-8 | 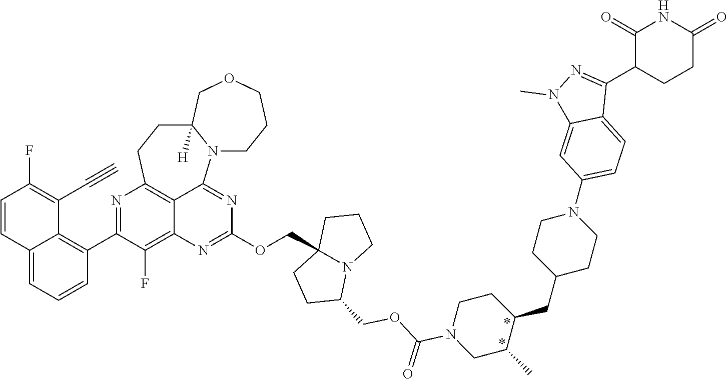 |
| 4-9 | 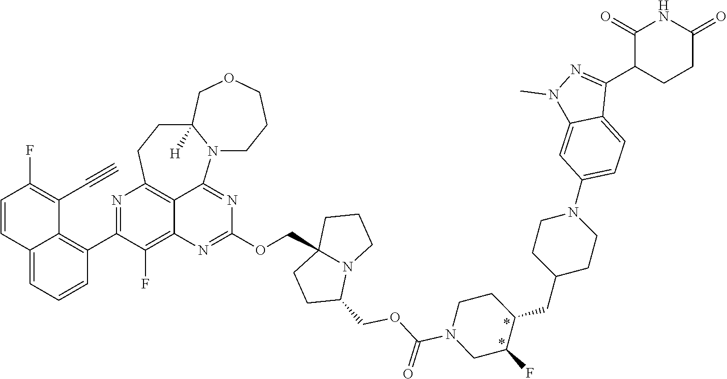 |

TABLE 3D2
Compounds

| | | | Changes to |
|---|---|---|---|
| | | | Procedure: |
| | | | Different |
| | | | Reagents/ |
| | | | Starting |
| Example | LCMS | ¹H-NMR (400 MHz, Methanol-d₄) | Materials |
| 4-2 | 1022.5 | δ 8.20-8.10 (m, 2H), 7.95 (dt, J = 9.6, 1.8 Hz, 1H), | INT 5-5 and |
| | | 7.84-7.79 (m, 1H), 7.75-7.64 (m, 2H), 7.52-7.44 | INT 27-1 |
| | | (m, 1H), 7.42-7.35 (m, 1H), 4.79-4.63 (m, 2H), | in step 1 |
| | | 4.62-4.23 (m, 4H), 4.22-3.82 (m, 10H), 3.80-3.67 | |
| | | (m, 2H), 3.65-3.55 (m, 1H), 3.47-3.37 (m, 1H), | |
| | | 3.28-3.20 (m, 2H), 3.14-3.04 (m, 1H), 2.86-2.69 | |
| | | (m, 4H), 2.50-2.04 (m, 14H), 2.03-1.88 (m, 3H), | |
| | | 1.79-1.55 (m, 3H), 1.38-1.23 (m, 7H), | |
| | | 1.13-1.00 (m, 2H). | |
| 4-3 | 1076.5 | δ 8.23-8.10 (m, 2H), 7.77-7.63 (m, 3H), 7.49 (t, J = | INT 5-5 and |
| | | 8.9 Hz, 1H), 7.34 (d, J = 2.4 Hz, 1H), 7.21 (dd, J = | INT 30-1 |
| | | 8.5, 2.6 Hz, 1H), 5.08 (dd, J = 12.5, 5.5 Hz, 1H), 4.82- | in step 1 |
| | | 4.73 (m, 1H), 4.72-4.64 (m, 1H), 4.63-4.44 (m, | |
| | | 2H), 4.41-4.23 (m, 2H), 4.19-3.95 (m, 8H), 3.80- | |
| | | 3.67 (m, 2H), 3.65-3.57 (m, 1H), 3.49-3.40 (m, | |
| | | 1H), 3.26-3.17 (m, 1H), 3.03-2.67 (m, 8H), 2.51- | |
| | | 2.32 (m, 4H), 2.32-2.05 (m, 9H), 2.04-1.95 (m, | |
| | | 1H), 1.89-1.49 (m, 6H), 1.34-0.99 (m, 6H). | |
| 4-4 | 1079.6 | δ 8.22-8.12 (m, 2H), 7.77-7.66 (m, 2H), 7.55- | INT 5-5 and |
| | | 7.44 (m, 2H), 7.13-7.03 (m, 1H), 4.82-4.73 (m, | INT 31-1 |
| | | 1H), 4.71-4.64 (m, 1H), 4.62-4.54 (m, 1H), 4.40- | in step 1 |
| | | 4.23 (m, 3H), 4.22-3.95 (m, 9H), 3.95-3.82 (m, | |
| | | 2H), 3.80-3.68 (m, 2H), 3.66-3.58 (m, 1H), 3.58- | |
| | | 3.51 (m, 2H), 3.48-3.41 (m, 1H), 3.13-3.00 (m, | |
| | | 3H), 2.91-2.69 (m, 4H), 2.54-2.05 (m, 14H), 2.05- | |
| | | 1.96 (m, 1H), 1.93-1.81 (m, 2H), 1.81-1.55 (m, | |
| | | 4H), 1.55-1.42 (m, 2H), 1.30-1.18 (m, 2H), 1.18- | |
| | | 1.02 (m, 2H). | |
| 4-5 | 1079.6 | δ 8.22-8.08 (m, 2H), 7.77-7.64 (m, 2H), 7.54- | INT 5-5 and |
| | | 7.41 (m, 1H), 6.95-6.65 (m, 2H), 4.81-4.73 (m, | INT 31-2 |
| | | 1H), 4.71-4.63 (m, 1H), 4.62-4.53 (m, 1H), 4.41- | in step 1 |
| | | 4.23 (m, 3H), 4.20-3.92 (m, 9H), 3.92-3.68 (m, | |
| | | 5H), 3.65-3.56 (m, 1H), 3.49-3.38 (m, 2H), 3.12- | |
| | | 3.05 (m, 1H), 3.05-2.92 (m, 2H), 2.88-2.66 (m, | |
| | | 4H), 2.50-2.04 (m, 14H), 2.03-1.96 (m, 1H), 1.93- | |
| | | 1.82 (m, 2H), 1.81-1.52 (m, 4H), 1.48-1.29 (m, | |
| | | 2H), 1.29-1.17 (m, 2H), 1.17-1.01 (m, 2H). | |
| 4-6 | 1075.6 | δ 8.22-8.12 (m, 2H), 7.95 (dd, J = 8.9, 3.1 Hz, 1H), | INT 5-5 and |
| | | 7.82-7.67 (m, 3H), 7.49 (t, J = 8.9 Hz, 1H), 7.40- | INT 35-1A in |
| | | 7.30 (m, 1H), 4.82-4.67 (m, 2H), 4.65-4.51 (m, | step 1 |
| | | 2H), 4.51-4.39 (m, 2H), 4.33-4.25 (m, 1H), 4.18- | |
| | | 3.99 (m, 7H), 3.99-3.85 (m, 3H), 3.83-3.71 (m, | |
| | | 4H), 3.71-3.56 (m, 3H), 3.49-3.43 (m, 1H), 3.26- | |
| | | 3.16 (m, 1H), 3.14-2.92 (m, 2H), 2.90-2.71 (m, | |
| | | 3H), 2.59-2.06 (m, 15H), 2.04-1.96 (m, 1H), 1.94- | |
| | | 1.60 (m, 5H), 1.56-1.20 (m, 4H), 0.92-0.84 | |
| | | (m, 3H). | |
| 4-7 | 1075.6 | δ 8.20-8.11 (m, 2H), 7.90 (d, J = 8.9 Hz, 1H), 7.76- | INT 5-5 and |
| | | 7.59 (m, 3H), 7.53-7.44 (m, 1H), 7.30 (d, J = 8.9 | INT 35-1C in |
| | | Hz, 1H), 4.81-4.74 (m, 1H), 4.71-4.63 (m, 1H), | step 1 |
| | | 4.63-4.52 (m, 2H), 4.42 (dd, J = 9.6, 5.1 Hz, 2H), | |
| | | 4.39-4.21 (m, 2H), 4.19-3.92 (m, 9H), 3.91-3.84 | |
| | | (m, 1H), 3.84-3.68 (m, 4H), 3.66-3.52 (m, 2H), | |
| | | 3.13-3.05 (m, 1H), 2.89-2.72 (m, 3H), 2.59-2.06 | |
| | | (m, 16H), 2.04-1.94 (m, 2H), 1.93-1.77 (m, 2H), | |
| | | 1.75-1.48 (m, 3H), 1.39-1.03 (m, 5H), 1.01-0.87 | |
| | | (m, 3H). | |
| 4-8 | 1075.6 | δ 8.21-8.08 (m, 2H), 7.86 (d, J = 8.9 Hz, 1H), 7.76- | INT 5-5 and |
| | | 7.64 (m, 2H), 7.62-7.38 (m, 2H), 7.31-7.19 (m, | INT 35-1D in |
| | | 1H), 4.82-4.73 (m, 1H), 4.71-4.52 (m, 3H), 4.51- | step 1 |
| | | 4.38 (m, 2H), 4.37-4.23 (m, 2H), 4.19-3.91 (m, | |
| | | 9H), 3.91-3.68 (m, 5H), 3.65-3.57 (m, 1H), 3.42 | |
| | | (d, J = 27.7 Hz, 3H), 3.25-3.18 (m, 1H), 3.12-3.04 | |
| | | (m, 1H), 2.90-2.72 (m, 3H), 2.57-2.04 (m, 16H), | |
| | | 1.91-1.74 (m, 2H), 1.72-1.44 (m, 3H), 1.34-1.06 | |
| | | (m, 4H), 1.00-0.81 (m, 3H). | |
| 4-9 | 1079.6 | δ 8.20-8.11 (m, 2H), 7.90 (d, J = 8.8 Hz, 1H), 7.76- | INT 5-5 and |
| | | 7.60 (m, 3H), 7.52-7.43 (m, 1H), 7.30 (dd, J = 9.1, | INT 34-1D in |
| | | 1.9 Hz, 1H), 4.83-4.64 (m, 2H), 4.63-4.49 (m, 2H), | step 1 |
| | | 4.42 (dd, J = 9.5, 5.1 Hz, 2H), 4.37-4.11 (m, 4H), | |
| | | 4.11-4.03 (m, 5H), 4.03-3.67 (m, 8H), 3.65-3.58 | |
| | | (m, 1H), 3.24-3.17 (m, 1H), 3.13-3.04 (m, 1H), | |
| | | 3.04-2.92 (m, 2H), 2.89-2.71 (m, 2H), 2.58-2.23 | |
| | | (m, 9H), 2.23-2.04 (m, 7H), 2.04-1.79 (m, 4H), | |
| | | 1.78-1.20 (m, 6H). | |

### Examples 5-1-5-12

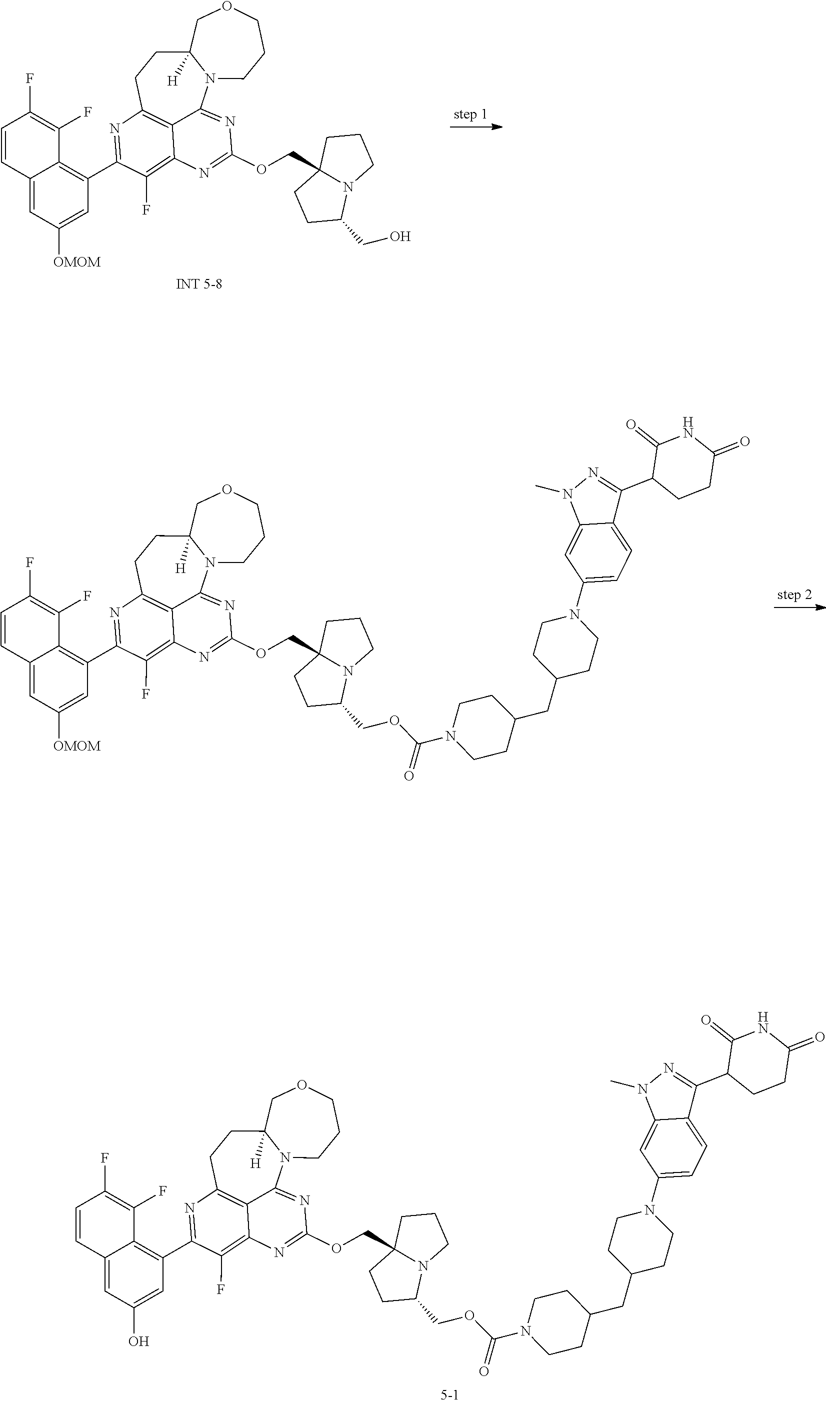

Step 1: ((3S,7aS)-7a-((((S)-2-(7,8-difluoro-3-(methoxymethoxy)naphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxopiperidin-3-yl)-1-methyl-1H-indazol-6-yl)piperidin-4-yl)methyl)piperidine-1-carboxylate. To a solution of INT 5-8 (0.04 mmol) in acetonitrile (1.0 mL) was added carbonyldiimidazole (0.12 mmol). The resulting mixture was stirred at 60° C. for 1 hour under an argon atmosphere. The reaction mixture was diluted with water (10 mL) and dichloromethane (10 mL). The aqueous layer was extracted with dichloromethane (3×10 mL). The organic layers were combined, washed with brine (10 mL), dried over anhydrous sodium sulfate, filtered, and concentrated under reduced pressure. The residue was dissolved in acetonitrile (1 mL) and to the reaction mixture was added 4-dimethylaminopyridine (0.04 mmol) followed by a solution of INT 14-3 (0.09 mmol) in acetonitrile (1.0 mL), dimethyl sulfoxide (0.25 mL), and diisopropylethylamine (0.2 mL). The reaction mixture was stirred at 70° C. overnight. The reaction mixture was then cooled to room temperature, concentrated, The reaction mixture was stirred at 70° C. overnight. The reaction mixture was then cooled to room temperature, concentrated, and the resulting residue was purified by silica gel chromatography eluting with 0% to 30% 10% ammonium hydroxide in ethanol/dichloromethane to yield the title compound. LCMS: 1115.6

Step 2: ((3S,7aS)-7a-((((S)-2-(7,8-difluoro-3-hydroxynaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxopiperidin-3-yl)-1-methyl-1H-indazol-6-yl)piperidin-4-yl)methyl)piperidine-1-carboxylate (Example 5-1). To a solution of ((3S,7aS)-7a-((((S)-2-(7,8-difluoro-3-(methoxymethoxy)naphthalen-1-yl)-1-fluoro-4,5,5a,6,9,10-hexahydro-8H-7-oxa-3,10a,11,13-tetraazanaphtho[l1,8-ab]heptalen-12-yl)oxy)methyl)hexahydro-1H-pyrrolizin-3-yl)methyl 4-((1-(3-(2,6-dioxopiperidin-3-yl)-1-methyl-1H-indazol-6-yl)piperidin-4-yl)methyl)piperidine-1-carboxylate INT 5-8 (0.04 mmol) in dichloromethane (3 mL) was added HCl (4 M in dioxane, 0.3 mL) dropwise at room temperature. The reaction mixture was stirred at room temperature for 20 minutes, then concentrated under reduced pressure. The residue was purified by RP-HPLC (0 to 50% 0.1% trifluoroacetic acid in acetonitrile/0.1% trifluoroacetic acid in water). LCMS: 1071.5 ¹H NMR (400 MHz, MeOD) δ 7.94 (dd, J=8.9, 2.3 Hz, 1H), 7.79-7.72 (m, 1H), 7.69-7.60 (m, 1H), 7.48-7.38 (m, 1H), 7.38-7.20 (m, 3H), 4.81-4.64 (m, 2H), 4.62-4.54 (m, 1H), 4.50-4.40 (m, 2H), 4.37-4.26 (m, 1H), 4.23-3.88 (m, 1OH), 3.88-3.68 (m, 4H), 3.68-3.56 (m, 3H), 3.26-3.09 (m, 2H), 2.91-2.70 (m, 4H), 2.58-2.04 (m, 16H), 2.03-1.94 (m, 1H), 1.94-1.83 (m, 1H), 1.83-1.55 (m, 5H), 1.38-1.26 (m, 2H), 1.20-1.05 (m, 2H).

The following Examples were made in a similar fashion to Example 5-1 and are shown below in Table 3E1. To prepare the below Examples, different reagents/starting materials were used than some of those described in the steps toward Example 5-1 and are noted in the last column of Table 3E2—“Changes to Procedure: Different Reagents/Starting Materials”. A person of ordinary skill in the art will readily recognize which reagents/starting materials used for the synthesis of Example 5-1 were replaced with the different reagents/starting materials noted below.

TABLE 3E1
Compounds

| Example | Structure |
|---|---|
| 5-2 | 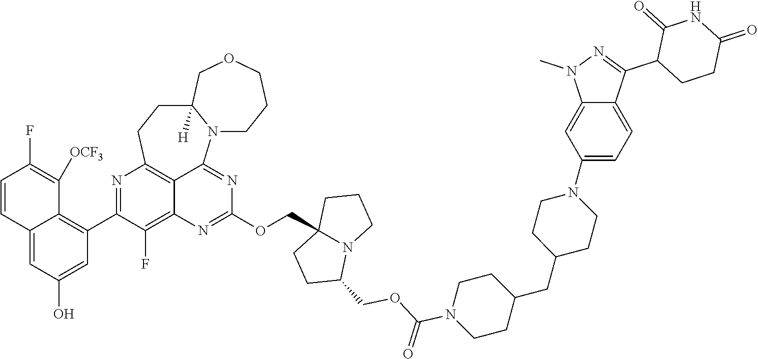 |
| 5-3 | 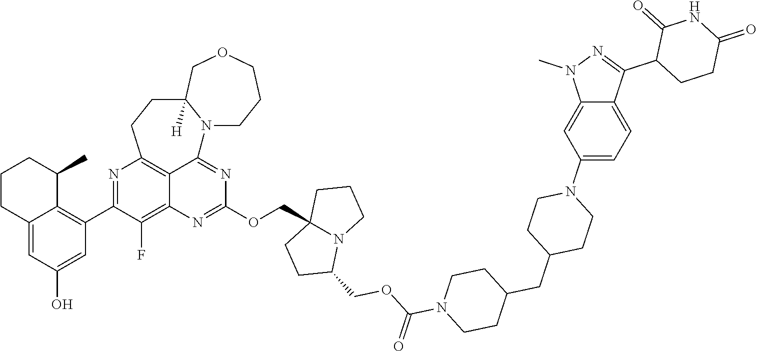 |
| 5-4 | 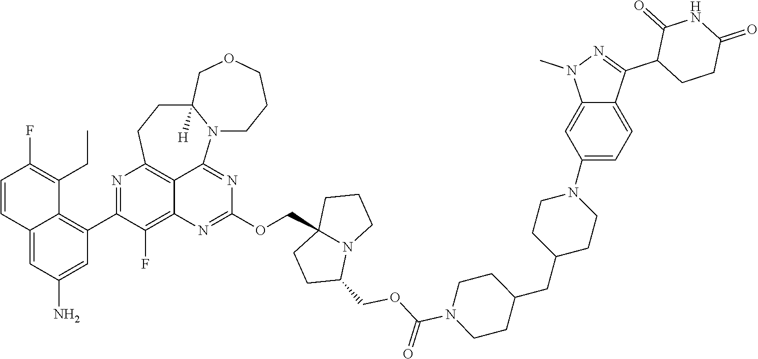 |
| 5-5 | 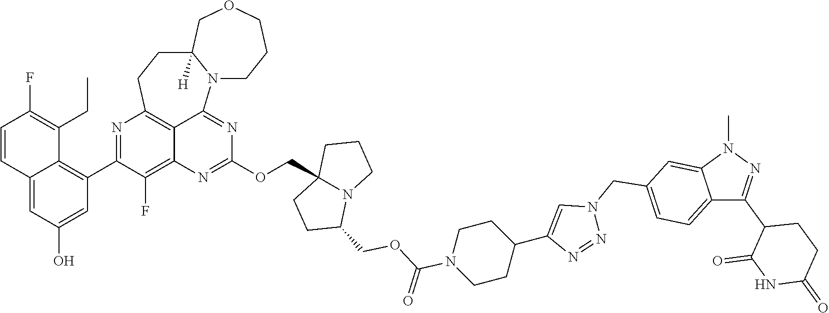 |
| 5-6 | 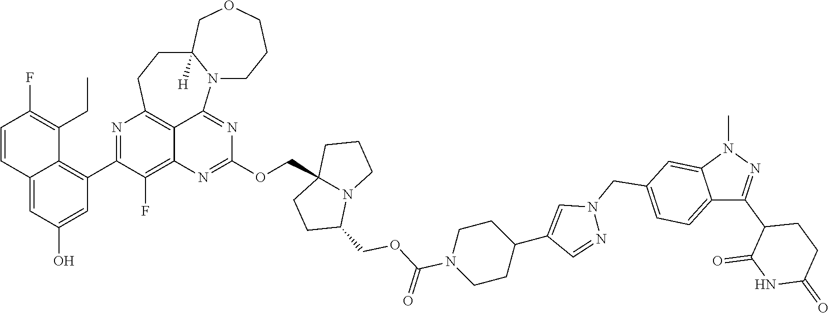 |
| 5-7 | 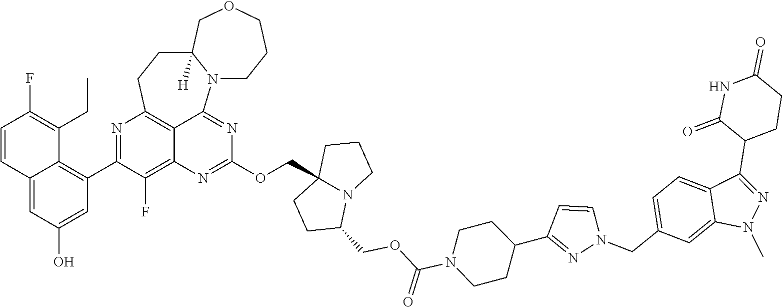 |
| 5-8 | 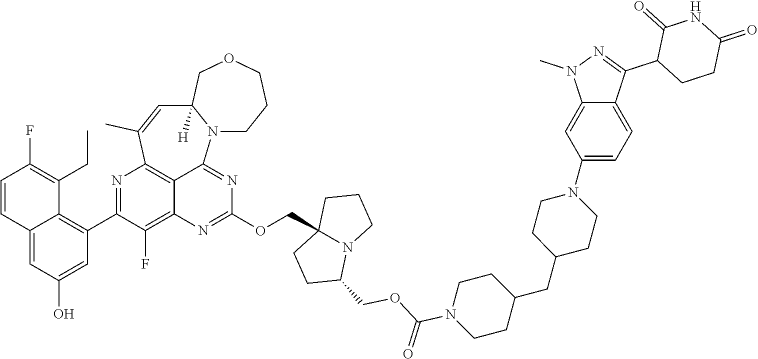 |
| 5-9 | 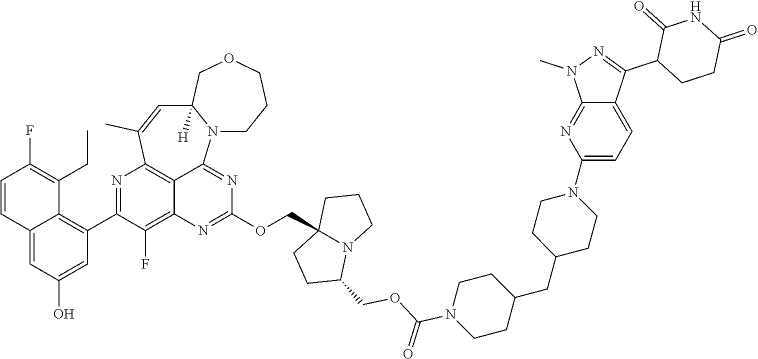 |
| 5-10 | 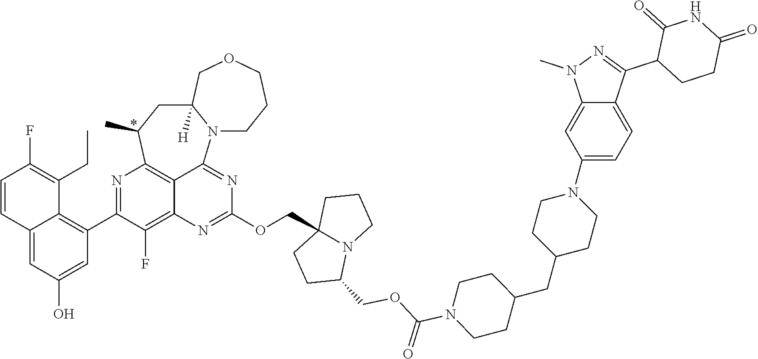 |
| 5-11 | 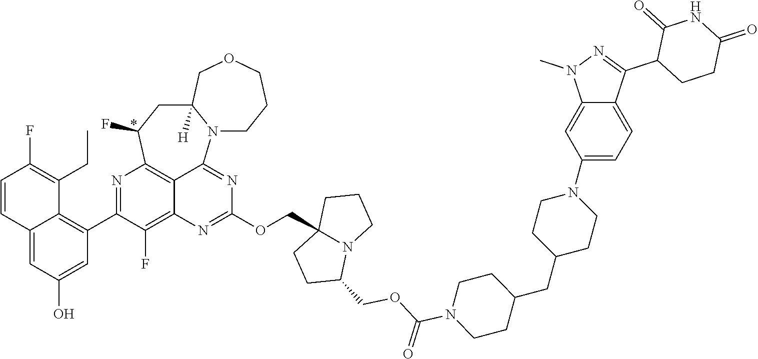 |
| 5-12 | 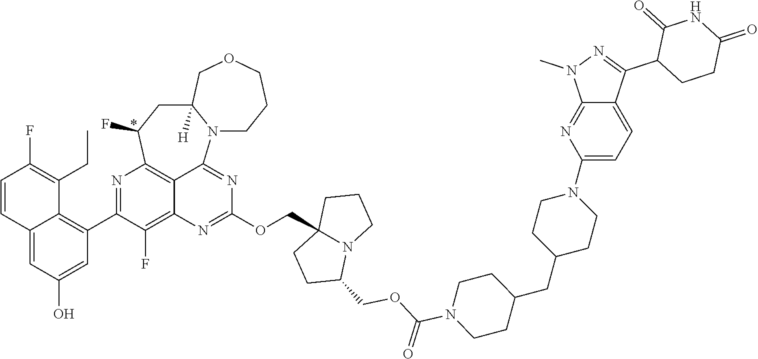 |

TABLE 3E2
Compounds

| | | | Changes to |
|---|---|---|---|
| | | | Procedure: |
| | | | Different |
| | | | Reagents/ |
| | | | Starting |
| Example | LCMS | ¹H-NMR (400 MHz, Methanol-d₄) | Materials |
| 5-2 | 1137.6 | δ 7.95-7.84 (m, 2H), 7.73-7.61 (m, 1H), 7.53- | INT 5-9 |
| | | 7.37 (m, 3H), 7.34-7.27 (m, 1H), 4.81-4.73 (m, | in step 1 |
| | | 1H), 4.68-4.61 (m, 1H), 4.61-4.47 (m, 2H), 4.47- | |
| | | 4.27 (m, 3H), 4.21-4.11 (m, 2H), 4.11-3.96 (m, | |
| | | 6H), 3.96-3.84 (m, 1H), 3.82-3.67 (m, 4H), 3.64- | |
| | | 3.52 (m, 3H), 3.23-3.08 (m, 2H), 2.92-2.71 (m, | |
| | | 4H), 2.57-2.22 (m, 9H), 2.22-2.04 (m, 7H), 2.04- | |
| | | 1.93 (m, 1H), 1.93-1.56 (m, 6H), 1.38-1.27 (m, | |
| | | 2H), 1.22-1.06 (m, 2H). | |
| 5-3 | 1053.6 | δ 7.91 (d, J = 8.7 Hz, 1H), 7.73-7.62 (m, 1H), 7.31 | INT 5-10 |
| | | (d, J = 8.9 Hz, 1H), 6.67 (d, J = 2.6 Hz, 1H), 6.62 (d, | in step 1 |
| | | J = 2.7 Hz, 1H), 4.78 (d, J = 12.4 Hz, 1H), 4.65 (d, J = | |
| | | 12.2 Hz, 1H), 4.60-4.53 (m, 1H), 4.50-4.38 (m, | |
| | | 2H), 4.37-4.27 (m, 1H), 4.24-4.11 (m, 2H), 4.08 | |
| | | (s, 3H), 4.06-3.87 (m, 4H), 3.84-3.67 (m, 4H), 3.64- | |
| | | 3.51 (m, 3H), 3.49-3.39 (m, 1H), 3.18-3.06 (m, | |
| | | 3H), 2.94-2.71 (m, 6H), 2.57-2.24 (m, 8H), 2.19- | |
| | | 2.04 (m, 7H), 2.01-1.57 (m, 12H), 1.33 (t, J = 7.0 | |
| | | Hz, 2H), 1.21-1.07 (m, 2H), 0.83 (d, J = 7.0 Hz, 3H). | |
| 5-4 | 1080.6 | δ 7.90 (d, J = 8.6 Hz, 1H), 7.84-7.73 (m, 1H), 7.69- | INT 5-12 |
| | | 7.60 (m, 1H), 7.60-7.53 (m, 1H), 7.38-7.15 (m, | in step 1 |
| | | 3H), 4.83-4.72 (m, 1H), 4.72-4.53 (m, 3H), 4.53- | |
| | | 4.26 (m, 4H), 4.24-4.11 (m, 2H), 4.10-3.89 (m, | |
| | | 7H), 3.84-3.67 (m, 4H), 3.65-3.51 (m, 2H), 2.91- | |
| | | 2.72 (m, 4H), 2.58-1.93 (m, 20H), 1.90-1.56 (m, | |
| | | 7H), 1.35-1.26 (m, 2H), 1.20-1.06 (m, 2H), 0.93- | |
| | | 0.79 (m, 3H). | |
| 5-5 | 1065.5 | δ 7.81-7.63 (m, 3H), 7.59-7.48 (m, 1H), 7.35- | INT 5-4 and |
| | | 7.30 (m, 1H), 7.26 (td, J = 9.4, 4.3 Hz, 1H), 7.15- | INT 32-1 |
| | | 7.02 (m, 2H), 5.75-5.59 (m, 2H), 4.81-4.70 (m, | in step 1 |
| | | 1H), 4.70-4.47 (m, 3H), 4.43-4.23 (m, 3H), 4.23- | |
| | | 4.09 (m, 2H), 4.09-3.82 (m, 7H), 3.81-3.54 (m, | |
| | | 3H), 3.49-3.39 (m, 1H), 3.26-3.04 (m, 2H), 3.02- | |
| | | 2.84 (m, 3H), 2.84-2.66 (m, 2H), 2.52-2.04 (m, | |
| | | 15H), 2.03-1.84 (m, 3H), 1.67-1.50 (m, 2H), 0.88 | |
| | | (t, J = 7.4 Hz, 2H), 0.84-0.74 (m, 1H). | |
| 5-6 | 1064.6 | δ 7.72-7.63 (m, 2H), 7.56-7.48 (m, 1H), 7.43- | INT 5-4 and |
| | | 7.35 (m, 2H), 7.33 (t, J = 2.5 Hz, 1H), 7.26 (td, J = | INT 33-1 |
| | | 9.4, 4.0 Hz, 1H), 7.17-7.05 (m, 1H), 7.02 (d, J = | in step 1 |
| | | 8.5 Hz, 1H), 5.47-5.32 (m, 2H), 4.81-4.71 (m, | |
| | | 1H), 4.71-4.46 (m, 3H), 4.44-4.23 (m, 3H), 4.22- | |
| | | 4.10 (m, 2H), 4.09-3.82 (m, 7H), 3.81-3.72 (m, | |
| | | 1H), 3.72-3.55 (m, 2H), 3.49-3.38 (m, 1H), 3.25- | |
| | | 3.04 (m, 2H), 3.00-2.61 (m, 5H), 2.52-2.04 (m, | |
| | | 15H), 2.02-1.78 (m, 3H), 1.57-1.38 (m, 2H), 0.88 | |
| | | (t, J = 7.4 Hz, 2H), 0.84-0.77 (m, 1H). | |
| 5-7 | 1064.6 | δ 7.73-7.55 (m, 3H), 7.37-7.30 (m, 2H), 7.29- | INT 5-4 and |
| | | 7.22 (m, 1H), 7.16-6.96 (m, 2H), 6.20-6.12 (m, | INT 33-2 |
| | | 1H), 5.47-5.38 (m, 2H), 4.81-4.71 (m, 1H), 4.71- | in step 1 |
| | | 4.46 (m, 3H), 4.44-4.25 (m, 3H), 4.25-4.13 (m, | |
| | | 2H), 4.09-3.91 (m, 6H), 3.91-3.82 (m, 1H), 3.82- | |
| | | 3.72 (m, 1H), 3.72-3.55 (m, 2H), 3.49-3.39 (m, | |
| | | 1H), 3.25-3.04 (m, 2H), 3.02-2.62 (m, 5H), 2.54- | |
| | | 2.06 (m, 15H), 2.03-1.78 (m, 3H), 1.70-1.52 (m, | |
| | | 2H), 0.88 (t, J = 7.4 Hz, 2H), 0.84-0.76 (m, 1H). | |
| 5-8 | 1093.6 | δ 7.91 (d, J = 8.8 Hz, 1H), 7.73-7.61 (m, 2H), 7.35- | INT 5-14 |
| | | 7.21 (m, 3H), 7.21-7.00 (m, 1H), 6.26-6.17 (m, | in step 1 |
| | | 1H), 4.80-4.59 (m, 3H), 4.50-4.39 (m, 2H), 4.38- | |
| | | 4.25 (m, 2H), 4.22-3.99 (m, 10H), 3.85-3.71 (m, | |
| | | 3H), 3.68-3.49 (m, 3H), 2.91-2.70 (m, 4H), 2.63- | |
| | | 2.48 (m, 2H), 2.48-2.31 (m, 4H), 2.31-2.21 (m, | |
| | | 3H), 2.19-1.98 (m, 11H), 1.91-1.57 (m, 6H), 1.38- | |
| | | 1.23 (m, 2H), 1.18-1.04 (m, 2H), 0.88-0.77 (m, | |
| | | 3H). | |
| 5-9 | 1094.6 | δ 7.70 (d, J = 9.1 Hz, 1H), 7.62-7.50 (m, 1H), 7.24- | INT 5-14 and |
| | | 7.09 (m, 2H), 7.09-6.89 (m, 1H), 6.66-6.57 (m, | INT 37-1 |
| | | 1H), 6.14-6.04 (m, 1H), 4.67-4.46 (m, 3H), 4.46- | in step 1 |
| | | 4.32 (m, 3H), 4.24-3.86 (m, 10H), 3.78 (s, 3H), 3.71- | |
| | | 3.63 (m, 1H), 3.52-3.42 (m, 1H), 2.87-2.73 (m, | |
| | | 2H), 2.72-2.57 (m, 4H), 2.51-2.41 (m, 1H), 2.38- | |
| | | 2.24 (m, 4H), 2.24-2.09 (m, 4H), 2.07-1.86 (m, | |
| | | 9H), 1.72-1.41 (m, 6H), 1.15-1.00 (m, 4H), 1.00- | |
| | | 0.87 (m, 2H), 0.75-0.64 (m, 3H). | |
| 5-10 | 1094.8 | δ 7.82 (d, J = 8.8 Hz, 1H), 7.63-7.53 (m, 2H), 7.26- | INT 5-18 |
| | | 7.10 (m, 3H), 7.05 (m, 1H), 4.70-4.44 (m, 4H), | in step 1 |
| | | 4.40-4.14 (m, 4H), 4.12-3.75 (m, 10H), 3.72-3.55 | |
| | | (m, 4H), 3.50 (m, 3H), 3.34 (m, 1H), 2.78-2.62 (m, | |
| | | 3H), 2.54-2.21 (m, 7H), 2.21-2.10 (m, 2H), 2.10- | |
| | | 1.93 (m, 7H), 1.91-1.82 (m, 2H), 1.71 (m, 2H), 1.56 | |
| | | (m, 4H), 1.32-1.21 (m, 6H), 1.00 (m, 2H), 0.84- | |
| | | 0.79 (m, 1H), 0.71 (dt, J = 25.7, 7.4 Hz, 3H). | |
| 5-11 | 1099.6 | δ 7.81 (d, J = 8.7 Hz, 1H), 7.66-7.50 (m, 2H), 7.25- | INT 5-19 |
| | | 7.18 (m, 2H), 7.18-7.09 (m, 1H), 7.07-6.93 (m, | in step 1 |
| | | 1H), 5.75 (dt, J = 46.5, 7.8 Hz, 1H), 4.69-4.60 (m, | |
| | | 1H), 4.60-4.52 (m, 1H), 4.47-4.35 (m, 2H), 4.32 | |
| | | (dd, J = 9.6, 5.1 Hz, 1H), 4.29-4.15 (m, 2H), 4.09- | |
| | | 3.95 (m, 6H), 3.94-3.82 (m, 3H), 3.81-3.72 (m, | |
| | | 1H), 3.70-3.59 (m, 3H), 3.54-3.41 (m, 3H), 2.89- | |
| | | 2.60 (m, 5H), 2.50-2.20 (m, 7H), 2.20-2.09 (m, | |
| | | 3H), 2.09-1.88 (m, 8H), 1.81-1.71 (m, 1H), 1.71- | |
| | | 1.44 (m, 5H), 1.20 (t, J = 6.8 Hz, 2H), 1.07-0.95 (m, | |
| | | 2H), 0.79-0.68 (m, 3H). | |
| 5-12 | | δ 7.70 (dd, J = 9.1, 1.7 Hz, 1H), 7.62-7.50 (m, 1H), | INT 5-19 and |
| | | 7.24-7.18 (m, 1H), 7.18-7.09 (m, 1H), 7.07-6.92 | INT 37-1 |
| | | (m, 1H), 6.62 (d, J = 9.2 Hz, 1H), 5.75 (dt, J = 46.5, | in step 1 |
| | | 7.8 Hz, 1H), 4.59-4.50 (m, 1H), 4.47-4.33 (m, 4H), | |
| | | 4.29-4.14 (m, 3H), 4.10 (dd, J = 9.4, 5.1 Hz, 1H), | |
| | | 4.07-3.95 (m, 3H), 3.95-3.82 (m, 3H), 3.81-3.71 | |
| | | (m, 4H), 3.67-3.59 (m, 1H), 3.53-3.43 (m, 1H), | |
| | | 2.89-2.56 (m, 8H), 2.40-2.24 (m, 5H), 2.23-2.08 | |
| | | (m, 4H), 2.08-1.91 (m, 6H), 1.70-1.44 (m, 6H), | |
| | | 1.14-1.02 (m, 4H), 1.02-0.90 (m, 2H), 0.78-0.66 | |
| | | (m, 3H). | |

### Examples 6-1-6-4

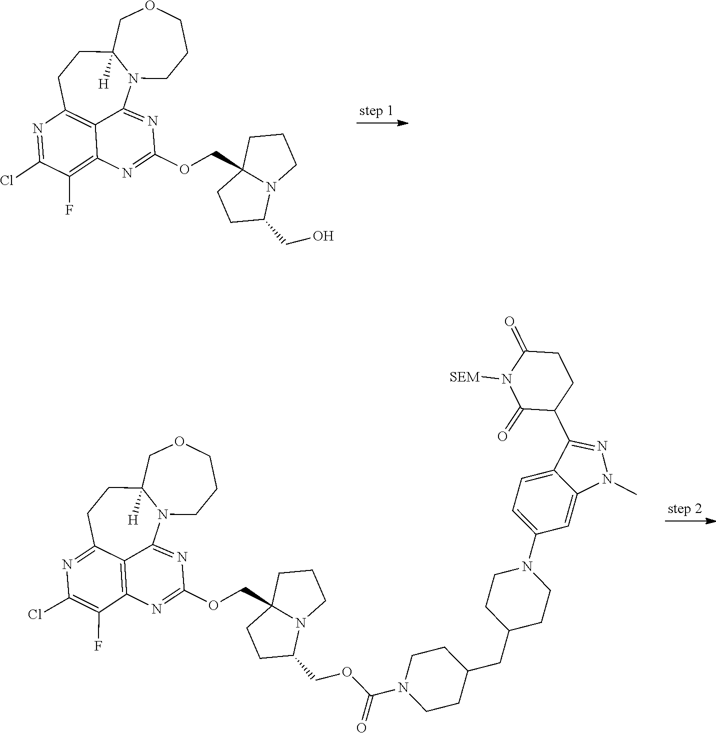

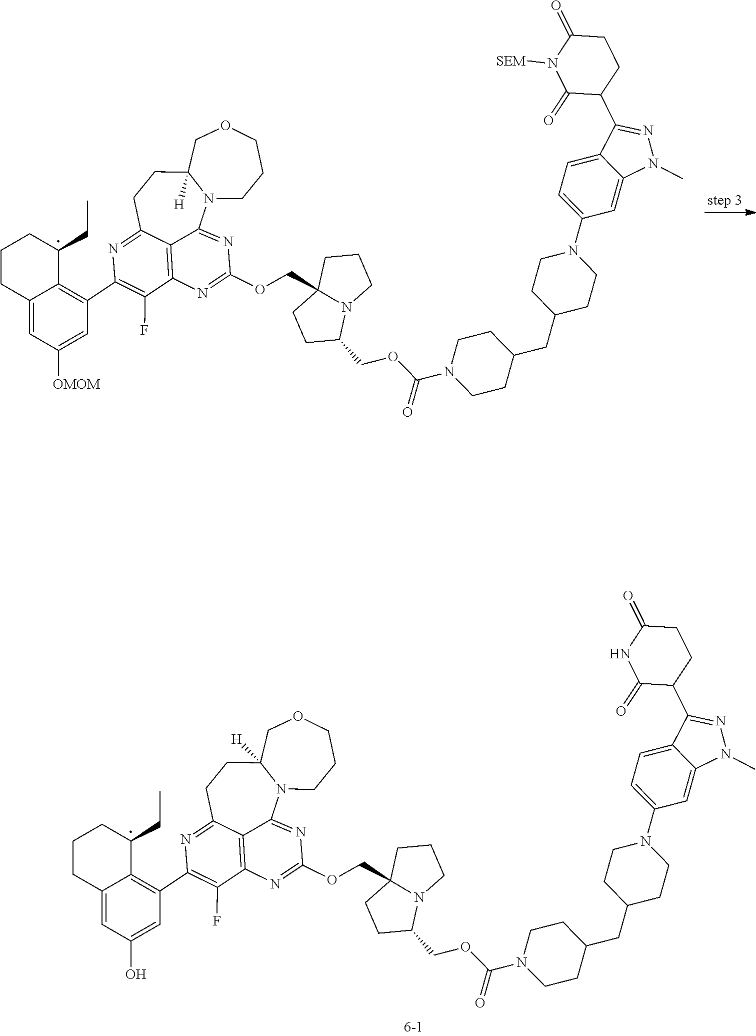

Step 1: ((3S,7aS)-7a-((((S)-2-chloro-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxo-1-((2-(trimethylsilyl)ethoxy)methyl)piperidin-3-yl)-1-methyl-1H-indazol-6-yl)piperidin-4-yl)methyl)piperidine-1-carboxylate-1-methane (1/1). To a solution of ((3S,7aS)-7a-((((S)-2-chloro-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methanol-methane (1/1) (854 mg, 1.49 mmol) in acetonitrile (14 mL) was added carbonyldiimidazole (4.5 mmol). The resulting mixture was stirred at 60° C. for 1 hour under an argon atmosphere. The reaction mixture was diluted with water (20 mL) and dichloromethane (20 mL). The aqueous layer was extracted with dichloromethane (3×20 mL). The organic layers were combined, washed with brine (20 mL), dried over anhydrous sodium sulfate, filtered, and concentrated under reduced pressure. The residue was dissolved in acetonitrile (14 mL) and to the reaction mixture was added 4-dimethylaminopyridine (1.5 mmol). To the reaction mixture was added a solution of 3-(1-methyl-6-(4-(piperidin-4-ylmethyl)piperidin-1-yl)-1H-indazol-3-yl)-1-((2-(trimethylsilyl)ethoxy)methyl)piperidine-2,6-dione INT 47-1 (3.0 mmol) and N,N-diisopropylethylamine (0.65 mL). The reaction mixture was stirred at 75° C. overnight. The reaction mixture was then cooled to room temperature, concentrated, and the resulting residue was purified by reverse phase column chromatography eluting with water/MeCN to yield the title compound. LCMS: 1057.9.

Step 2: ((3S,7aS)-7a-((((S)-2-((R*)-8-ethyl-3-(methoxymethoxy)-5,6,7,8-tetrahydronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxo-1-((2-(trimethylsilyl)ethoxy)methyl)piperidin-3-yl)-1-methyl-1H-indazol-6-yl)piperidin-4-yl)methyl)piperidine-1-carboxylate-1-methane (1/1). A solution of ((3S,7aS)-7a-((((S)-2-chloro-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxo-1-((2-(trimethylsilyl)ethoxy)methyl)piperidin-3-yl)-1-methyl-1H-indazol-6-yl)piperidin-4-yl)methyl)piperidine-1-carboxylate-1-methane (1/1) (0.028 mmol) and (R*)-2-(8-ethyl-3-(methoxymethoxy)-5,6,7,8-tetrahydronaphthalen-1-yl)-4,4,5,5-tetramethyl-1,3,2-dioxaborolane (INT 1-4) (0.085 mmol) in 1,4-dioxane (0.8 mL) was sparged with Ar for 5 min. Cs₂CO₃ (0.085 mmol) and Pd(PPh₃)₄(0.00425 mmol) were added, and the mixture was sparged for an additional 5 minutes. The reaction vial was sealed and stirred at 80° C. for 16 h. The mixture was then filtered through celite, rinsed with EtOAc, and the filtrate was concentrated to yield the title compound which was used in the next step without further purification. LCMS: 1241.7

Step 3: ((3S,7aS)-7a-((((S)-2-((R*)-8-ethyl-3-hydroxy-5,6,7,8-tetrahydronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxopiperidin-3-yl)-1-methyl-1H-indazol-6-yl)piperidin-4-yl)methyl)piperidine-1-carboxylate-1-methane (1/1). To a solution of ((3S,7aS)-7a-((((S)-2-((R*)-8-ethyl-3-(methoxymethoxy)-5,6,7, 8-tetrahydronaphthalen-1-yl)-1-fluoro-4,5,5a,6,9,10-hexahydro-8H-7-oxa-3,10a,11,13-tetraazanaphtho[1,8-ab]heptalen-12-yl)oxy)methyl)hexahydro-1H-pyrrolizin-3-yl)methyl 4-((1-(3-(2,6-dioxo-1-((2-(trimethylsilyl)ethoxy)methyl)piperidin-3-yl)-1-methyl-1H-indazol-6-yl)piperidin-4-yl)methyl)piperidine-1-carboxylate-1-methane (1/1) in DCM (1 mL) was added TFA (0.5 mL) at room temperature. The mixture was stirred at room temperature for 30 minutes, then concentrated and co-evaporated three timed with MeCN (5 mL). The residue was dissolved in MeOH (1 mL) and aqueous ammonium hydroxide solution (28-30%, 0.3 mL) was added. The reaction mixture was stirred at room temperature for 20 min, then concentrated under reduced pressure. The residue was purified by RP-HPLC (0 to 50%, 0.1% trifluoroacetic acid in acetonitrile/0.1% trifluoroacetic acid in water). Fractions containing the product were pooled and lyophilized to yield the title compound. LCMS: 1067.6 NMR: ¹H NMR (400 MHz, MeOD) δ 7.81 (d, J=8.8 Hz, 1H), 7.60 (s, 1H), 7.21 (d, J=9.3 Hz, 1H), 6.59-6.49 (m, 2H), 4.66 (d, J=12.3 Hz, 1H), 4.53 (d, J=12.3 Hz, 1H), 4.49-4.41 (m, 1H), 4.39-4.28 (m, 2H), 4.27-4.16 (m, 1H), 4.11-3.99 (m, 2H), 3.97 (s, 3H), 3.96-3.84 (m, 3H), 3.84-3.75 (m, 1H), 3.71-3.54 (m, 4H), 3.53-3.42 (m, 3H), 3.37-3.28 (m, 11H), 3.11-2.94 (m, 2H), 2.82-2.59 (m, 7H), 2.46-2.34 (m, 11H), 2.33-2.10 (m, 7H), 2.09-1.92 (m, 7H), 1.90-1.46 (m, 12H), 1.30-1.14 (m, 3H), 1.11-0.96 (m, 3H), 0.41 (t, J=7.4 Hz, 3H).

The following Examples were made in a similar fashion to Example 6-1 and are shown below in Table 3F1. To prepare the below Examples, different reagents/starting materials were used than some of those described in the steps toward Example 6-1 and are noted in the last column of Table 3F2—“Changes to Procedure: Different Reagents/Starting Materials”. A person of ordinary skill in the art will readily recognize which reagents/starting materials used for the synthesis of Example 6-1 were replaced with the different reagents/starting materials noted below.

TABLE 3F1
Compounds

| Example | Structure |
|---|---|
| 6-2 | 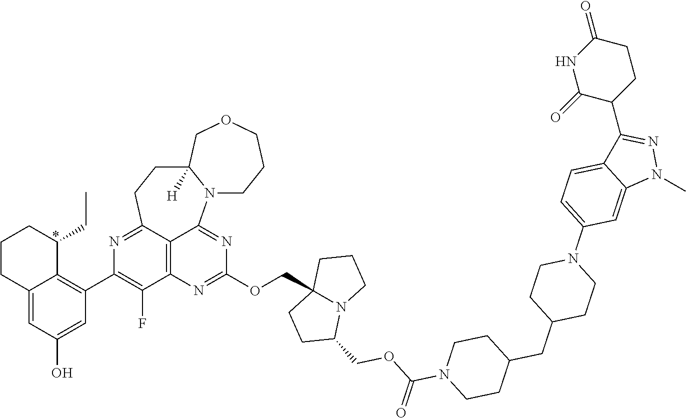 |
| 6-3 | 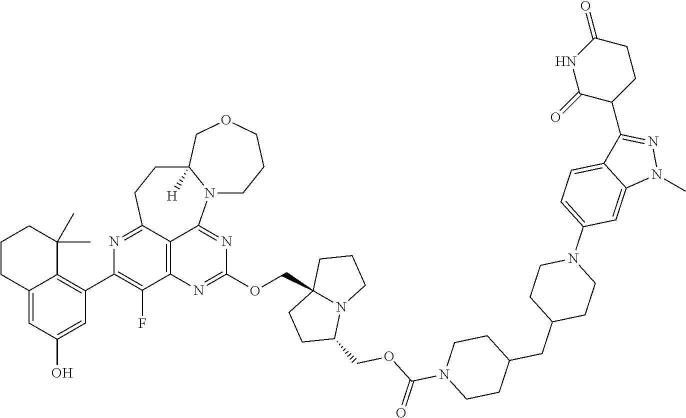 |
| 6-4 | 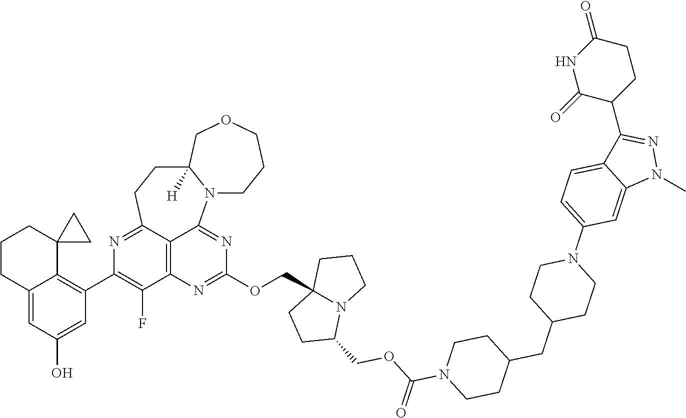 |

TABLE 3F2
Compounds

| | | | Changes to |
|---|---|---|---|
| | | | Procedure: |
| | | | Different |
| | | | Reagents/ |
| | | | Starting |
| Example | LCMS | ¹H-NMR (400 MHz, Methanol-d₄) | Materials |
| 6-2 | 1067.6 | δ 7.80 (d, J = 8.9 Hz, 1H), 7.57 (s, 1H), 7.20 (d, J = | INT 1-5 |
| | | 8.8 Hz, 1H), 6.59-6.47 (m, 2H), 4.69-4.51 (m, 2H), | in step 2 |
| | | 4.49-4.39 (m, 2H), 4.39-4.27 (m, 2H), 4.27-4.13 | |
| | | (m, 2H), 4.11-3.99 (m, 2H), 3.99-3.92 (m, 4H), | |
| | | 3.92-3.84 (m, 2H), 3.84-3.75 (m, 1H), 3.72-3.63 | |
| | | (m, 3H), 3.63-3.55 (m, 1H), 3.54-3.40 (m, 3H), | |
| | | 2.78-2.61 (m, 6H), 2.61-2.52 (m, 1H), 2.46-2.12 | |
| | | (m, 8H), 2.12-1.93 (m, 7H), 1.90-1.45 (m, 12H), | |
| | | 1.26-1.18 (m, 3H), 1.18-0.94 (m, 4H), 0.43 (t, J = | |
| | | 7.4 Hz, 3H). | |
| 6-3 | 1067.6 | δ 7.75 (d, J = 9.0 Hz, 1H), 7.49-7.38 (m, 1H), 7.15 | INT 1-6 |
| | | (d, J = 9.0 Hz, 1H), 6.60-6.48 (m, 1H), 6.36-6.20 | in step 2 |
| | | (m, 1H), 4.70-4.60 (m, 1H), 4.56-4.40 (m, 3H), | |
| | | 4.40-4.32 (m, 1H), 4.29 (dd, J = 9.5, 5.1 Hz, 1H), | |
| | | 4.24-4.15 (m, 1H), 4.13-4.00 (m, 2H), 3.95 (s, 3H), | |
| | | 3.93-3.83 (m, 3H), 3.83-3.76 (m, 1H), 3.75-3.54 | |
| | | (m, 5H), 3.53-3.43 (m, 1H), 2.83-2.58 (m, 7H), | |
| | | 2.46-2.10 (m, 9H), 2.10-1.90 (m, 8H), 1.80-1.42 | |
| | | (m, 11H), 1.27-1.14 (m, 3H), 1.11-0.97 (m, 3H), | |
| | | 0.95-0.80 (m, 5H). | |
| 6-4 | 1065.6 | δ 7.74 (d, J = 8.8 Hz, 1H), 7.51-7.34 (m, 1H), 7.14 | INT 1-7 |
| | | (d, J = 8.9 Hz, 1H), 6.73-6.32 (m, 2H), 4.69-4.32 | in step 2 |
| | | (m, 6H), 4.32-4.26 (m, 1H), 4.10-3.91 (m, 7H), | |
| | | 3.91-3.84 (m, 2H), 3.84-3.76 (m, 1H), 3.74-3.63 | |
| | | (m, 3H), 3.63-3.55 (m, 1H), 3.54-3.44 (m, 1H), | |
| | | 3.37-3.27 (m, 3H), 2.84-2.53 (m, 7H), 2.44-2.09 | |
| | | (m, 9H), 2.09-1.78 (m, 11H), 1.78-1.41 (m, 9H), | |
| | | 1.23-1.16 (m, 3H), 1.09-0.91 (m, 2H). | |

### XI. Biological Examples

### AsPC-1 KRAS G12D HiBit Assay

AsPC-1 KRAS(G12D)-HiBit cells (Promega product #CS3023190) were maintained in growth medium containing RPMI 1640 medium (Gibco product #32404-014) supplemented with GlutaMAX (Gibco product #35050-061), penicillin/streptomycin (Gibco product #15140-122) and 10% fetal bovine serum (HyClone product #SH30396.03). Cells were passaged 2 times per week to maintain sub-confluent densities. Passages 5 to 20 were used for assays.

Test compounds were prepared in 100% DMSO in 384-well polypropylene plates (Greiner Bio-One, product #784201) with 8 compounds per plate in grouped replicates of 4 at 10 serially diluted concentrations (1:3). The serially diluted compounds were transferred to low dead volume Echo plates (Beckman Coulter, product #001-12782).

Serially diluted test compounds were transferred to black 384-well cell culture-treated plates (Greiner Bio-One, product #781086) at 200 nL per well using an Echo acoustic dispenser (Beckman Coulter). AsPC-1 KRAS-HiBit cells were harvested and suspended in growth medium (described above). The cells were seeded in the assay plates containing test compounds at 4,000 cells per well in 40 μL. The plates containing cells and test compounds were incubated at 37° C., 5% CO₂ for 24 hours. The cells were then equilibrated to room temperature for 30 minutes and Nano-Glo HiBiT Lytic reagent (Promega, product #N3050) was prepared according to manufacturer protocol and equilibrated to room temperature. Medium (20 μL per well) was aspirated from assay plates using a BioTek EL406 washer dispenser (Agilent), and 20 μL of Nano-Glo HiBiT Lytic reagent was added per well. The plates were placed on an orbital shaker at 1000 rpm for 3 minutes, and incubated at room temperature for an additional 7 minutes before reading luminescence on an EnVision multimode microplate reader (Revvity).

The luminescence signal was normalized to negative (DMSO, 100%) and positive (no cells, 0%) controls and plotted against log 10 concentration of test compounds. The data was fitted to a 4 variables non-linear regression analysis to determine DC₅₀ and Dₘₐₓ (Table 4). DC₅₀ is defined as the concentration at which the mid-point between the top and bottom plateaus is reached. Dₘₐₓ is defined as 100% minus the lowest normalized data observed across the dose response (maximal signal loss). Unless otherwise noted, Dₘₐₓ standard deviation was less than 5. (*Dₘₐₓ standard deviation of 5-10)

TABLE 4
Biological data

| | DC₅₀ | Dₘₐₓ |
|---|---|---|
| Example | (nM) | (%) |
| 1-1 | 5.5 | 62 |
| 1-2 | 1.6 | 51 |
| 1-3 | 2.2 | 70 |
| 1-4 | 25.1 | 52 |
| 1-5 | 2.2 | 58 |
| 1-6* | 1.4 | 53 |
| 1-7 | 3.1 | 52 |
| 1-8 | 11.8 | 45 |
| 1-9 | 3.5 | 69 |
| 1-10 | 5.8 | 66 |
| 1-11 | 1.7 | 55 |
| 1-12 | 1.7 | 67 |
| 1-13 | 12.4 | 60 |
| 1-14 | 4.4 | 62 |
| 1-15 | 0.9 | 67 |
| 1-16 | 2.1 | 75 |
| 1-17 | 4.8 | 59 |
| 1-18 | 0.8 | 69 |
| 1-19 | 1.7 | 79 |
| 1-20 | 1.0 | 78 |
| 1-21 | 4.7 | 62 |
| 1-22 | 2.0 | 77 |
| 1-23 | 2.8 | 76 |
| 1-24 | 15.9 | 61 |
| 1-25 | 3.0 | 74 |
| 1-26 | 9.0 | 71 |
| 1-27 | 2.4 | 74 |
| 1-28 | 6.5 | 73 |
| 1-29 | 10.2 | 65 |
| 1-30 | 9.8 | 60 |
| 1-31 | 3.2 | 44 |
| 1-32 | 10.5 | 70 |
| 2-1* | 42.9 | 68 |
| 2-2 | 23.7 | 68 |
| 2-3 | 45.0 | 64 |
| 2-4* | 10.0 | 61 |
| 2-5* | 51.2 | 51 |
| 2-6* | 31.0 | 70 |
| 2-7* | 44.2 | 75 |
| 2-8 | 20.7 | 71 |
| 2-9 | 39.8 | 45 |
| 2-10 | 17.7 | 77 |
| 2-11 | 5.6 | 74 |
| 2-12* | 32.0 | 61 |
| 2-13* | 12.1 | 50 |
| 2-14* | 22.4 | 75 |
| 2-15* | 9.8 | 73 |
| 2-16 | 7.9 | 72 |
| 2-17 | 45.5 | 44 |
| 2-18 | 83.4 | 72 |
| 2-19 | 20.1 | 47 |
| 2-20* | 7.6 | 69 |
| 2-21 | 21.4 | 63 |
| 2-22 | 149.1 | 48 |
| 2-23 | 21.9 | 66 |
| 2-24 | 6.9 | 67 |
| 2-25* | 69.4 | 59 |
| 2-26 | 94.1 | 64 |
| 2-27* | 6.6 | 60 |
| 2-28 | 85.8 | 55 |
| 2-29 | 28.8 | 71 |
| 3-1 | 13.5 | 77 |
| 4-1 | 21.4 | 74 |
| 4-2 | 90.3 | 52 |

### AsPC-1 KRAS G12D HiBit Assay

AsPC-1 KRAS(G12D)-HiBit cells (Promega product #CS3023190) were maintained in growth medium containing RPMI 1640 medium, no phenol red (Gibco product #11835030) supplemented with 1X penicillin/streptomycin/Glutamine (Corning product #30-009-CI) and 10% fetal bovine serum (HyClone product #SH30396.03). Cells were passaged 2 times per week to maintain sub-confluent densities. Passages 5 to 12 were used for assays.

Test compounds were prepared in 100% DMSO in 384-well polypropylene plates (Greiner Bio-One, product #784201) with 8 compounds per plate in grouped replicates of 4 at 10 serially diluted concentrations (1:3).

AsPC-1 KRAS-HiBit cells were harvested and suspended in growth medium (described above). The cells were seeded in black 384-well cell culture-treated plates (Greiner Bio-One, product #781086) at 5,000 cells per well in 80 μL. The plates containing cells were incubated at 37° C., 5% CO₂ for 24 hours prior to dosing 0.4 μL of the test compounds using a Biomek 17 (Beckman Coulter). The cells treated with compounds were incubated at 37° C., 5% CO₂ for 24 hours. The cells were then equilibrated to room temperature for 30 minutes and Nano-Glo HiBiT Lytic reagent (Promega, product #N3050) was prepared according to manufacturer protocol and equilibrated to room temperature. Medium was aspirated from assay plates until 20 μL remained using a BioTek EL406 washer dispenser (Agilent), and 20 μL of Nano-Glo HiBiT Lytic reagent was added per well. The plates were placed on an orbital shaker at 1000 rpm for 3 minutes, and incubated at room temperature for an additional 7 minutes before reading luminescence on an EnVision multimode microplate reader (Revvity).

The luminescence signal was normalized to negative (DMSO, 100%) and positive (no cells, 0%) controls and plotted against log 10 concentration of test compounds. The data was fitted to a 4 variables non-linear regression analysis to determine DC₅₀ and Dₘₐₓ (Table 5). DC₅₀ is defined as the concentration at which the mid-point between the top and bottom plateaus is reached. Dₘₐₓ is defined as 100% minus the lowest normalized data observed across the dose response (maximal signal loss). Unless otherwise noted, Dₘₐₓ standard deviation was less than 5. (*Dmax standard deviation of 5-10)

TABLE 5
Biological data

| | | Dₘₐₓ |
|---|---|---|
| Example | DC₅₀ (nM) | (%) |
| 1-33 | 1.4 | 81 |
| 1-34 | 0.7 | 82 |
| 1-35 | 1.1 | 72 |
| 1-36 | 0.9 | 72 |
| 1-37 | 1.0 | 76 |
| 1-38 | 0.4 | 74 |
| 1-39 | 0.5 | 73 |
| 1-40 | 7.2 | 76 |
| 1-41 | 9.6 | 77 |
| 1-42* | 3.1 | 57 |
| 1-43 | 2.5 | 68 |
| 1-44 | 1.5 | 71 |
| 1-45 | 11.8 | 36 |
| 1-46 | 1.4 | 82 |
| 1-47 | 3.1 | 67 |
| 1-48 | 1.6 | 77 |
| 1-49 | 19.5 | 53 |
| 2-30 | 21.2 | 71 |
| 2-31 | 10.4 | 70 |
| 2-32 | 12.8 | 69 |
| 2-33* | 10.0 | 72 |
| 2-34 | 14.1 | 67 |
| 2-35 | 9.1 | 75 |
| 2-36 | 16.3 | 74 |
| 2-37 | 12.9 | 68 |
| 2-38 | 61.1 | 59 |
| 2-39* | 21.0 | 62 |
| 2-40 | 45.8 | 67 |
| 2-41 | 13.3 | 52 |
| 2-42 | 14.6 | 69 |
| 2-43* | 7.4 | 75 |
| 2-44* | 12.0 | 73 |
| 2-45 | 11.1 | 73 |
| 2-46 | 17.7 | 68 |
| 2-47 | 34.0 | 68 |
| 2-48 | 14.9 | 53 |
| 2-49 | 14.4 | 63 |
| 2-50 | 25.0 | 63 |
| 2-51 | 7.9 | 73 |
| 2-52 | 10.1 | 57 |
| 2-53 | 6.6 | 68 |
| 2-54 | 7.8 | 60 |
| 2-55 | 33.5 | 53 |
| 2-56 | 9.4 | 72 |
| 2-57* | 11.3 | 68 |
| 2-58* | 22.5 | 55 |
| 2-59 | 9.7 | 64 |
| 2-60* | 28.1 | 55 |
| 2-61 | 13.4 | 58 |
| 2-62 | 44.9 | 41 |
| 2-63 | 15.9 | 64 |
| 2-64 | 19.7 | 43 |
| 2-65 | 11.0 | 68 |
| 2-66* | 16.4 | 62 |
| 2-67* | 15.4 | 48 |
| 2-68* | 10.8 | 72 |
| 2-69 | 13.3 | 64 |
| 2-70 | 21.2 | 61 |
| 2-71 | 29.8 | 73 |
| 2-72* | 22.0 | 65 |
| 2-73 | 56.4 | 56 |
| 2-74 | 20.3 | 69 |
| 2-75* | 14.3 | 65 |
| 2-76 | 126 | 21 |
| 2-77 | 161 | 63 |
| 2-78 | 41.1 | 27 |
| 2-79 | 30.6 | 52 |
| 2-80 | 30.8 | 55 |
| 2-81* | 27.3 | 69 |
| 2-82 | 34.5 | 55 |
| 3-2 | 2.1 | 81 |
| 4-3 | 44.0 | 69 |
| 4-4 | 20.0 | 65 |
| 4-5 | 32.6 | 71 |
| 4-6* | 14.7 | 68 |
| 4-7 | 19.4 | 67 |
| 4-8 | 11.0 | 75 |
| 4-9 | 5.3 | 73 |
| 5-1 | 2.8 | 70 |
| 5-2 | 2.9 | 75 |
| 5-3 | 3.8 | 75 |
| 5-4* | 21.4 | 59 |
| 5-5 | 56.9 | 60 |
| 5-6 | 30.1 | 67 |
| 5-7 | 19.1 | 74 |
| 5-8 | 2.9 | 67 |
| 5-9 | 3.5 | 69 |
| 5-10 | 3.0 | 67 |
| 5-11 | 1.5 | 78 |
| 5-12 | 1.4 | 83 |
| 6-1 | 0.9 | 76 |
| 6-2 | 44.2 | 20 |
| 6-3 | 1.8 | 71 |
| 6-4 | 2.0 | 59 |

### AsPC-1 KRAS G12D Cell Viability Assay

AsPC-1 KRAS(G12D) cells (ACH-000222) were maintained in growth medium containing RPMI 1640 medium with GlutaMax (Gibco, product #61870-036) supplemented with 1× penicillin/streptomycin (Gibco, product 15140-122) and 10% fetal bovine serum (HyClone, product #SH30396.03). The cell culture was passaged 2 times per week to maintain sub-confluent densities and used for assays within 7 weeks of initial thaw. Cells were harvested and suspended in growth medium and seeded at 1000 cells per well in 40 μL in 384-well, ultra-low binding, U-shaped microplates (Corning, product #4516). The plates were incubated at 37° C., 5% CO2 for 48 hours to form spheroids prior to dosing with test compounds. Test compounds were prepared in 100% DMSO and applied directly to cells in plates using a Hewlitt Packard Digital Dispenser D300e in a 15 point logarithmically distributed dose range from 10 μM to 50 pM in grouped triplicates. Cells treated with compound were incubated at 37° C., 5% CO2 for another 96 hours. The plates were then equilibrated to room temperature for 15 minutes and CellTiter-Glo 3D reagent (Promega, product #G9683) was added at 40 μL per well using a Biotek MultiFLo FX dispenser (Agilent) for a final volume of 80 μL. Plates were placed on an orbital shaker at 600 rpm for 5 minutes and incubated at room temperature for an additional 20 minutes before reading luminescence on a Biotek Neo2 plate reader (Agilent). The luminescence signal was normalized to negative (DMSO, 100%) and positive (4 μM staurosporine, 0%) controls and plotted against log 10 concentration of test compounds. The data was fitted to a 4 variables non-linear regression analysis to determine EC₅₀ (Table 6). EC₅₀ is defined as the concentration at which the mid-point between the top and bottom plateaus is reached.

TABLE 6
Biological data

| Examples | EC₅₀ (nM) |
|---|---|
| 1-1 | 3.2 |
| 1-2 | 1.3 |
| 1-3 | 1.5 |
| 1-4 | 7.3 |
| 1-5 | 1.0 |
| 1-6 | 0.7 |
| 1-7 | 3.1 |
| 1-8 | 7.7 |
| 1-9 | 5.4 |
| 1-10 | 4.4 |
| 1-11 | 1.1 |
| 1-12 | 1.4 |
| 1-15 | 1.0 |
| 1-16 | 1.6 |
| 1-18 | 0.8 |
| 1-19 | 1.2 |
| 1-20 | 0.5 |
| 1-22 | 0.5 |
| 1-23 | 0.9 |
| 1-25 | 1.0 |
| 1-26 | 2.7 |
| 1-27 | 0.7 |
| 1-28 | 2.9 |
| 1-29 | 2.1 |
| 1-30 | 6.1 |
| 1-31 | 1.5 |
| 1-32 | 2.7 |
| 1-41 | 11.3 |
| 2-1 | 17.2 |
| 2-2 | 10.8 |
| 2-3 | 17.5 |
| 2-4 | 1.4 |
| 2-5 | 34.1 |
| 2-6 | 7.1 |
| 2-7 | 13.0 |
| 2-8 | 14.2 |
| 2-9 | 102 |
| 2-10 | 4.5 |
| 2-11 | 6.2 |
| 2-12 | 21.8 |
| 2-13 | 5.9 |
| 2-14 | 3.4 |
| 2-15 | 2.4 |
| 2-16 | 2.8 |
| 2-17 | 45.5 |
| 2-18 | 42.9 |
| 2-19 | 14.3 |
| 2-20 | 6.2 |
| 2-21 | 27.6 |
| 2-22 | 305 |
| 2-24 | 4.2 |
| 2-25 | 4.6 |
| 2-26 | 87.9 |
| 2-27 | 2.8 |
| 2-28 | 22.2 |
| 2-29 | 18.6 |
| 2-39 | 5.0 |
| 2-40 | 18.9 |
| 2-41 | 13.1 |
| 2-46 | 2.2 |
| 2-47 | 5.8 |
| 2-48 | 20.7 |
| 2-57 | 2.4 |
| 2-58 | 9.1 |
| 2-59 | 1.3 |
| 2-60 | 3.1 |
| 2-69 | 3.9 |
| 2-75 | 8.0 |
| 2-78 | 164 |
| 2-79 | 25.0 |
| 2-81 | 8.1 |
| 3-2 | 0.9 |
| 4-3 | 14.0 |
| 4-1 | 6.0 |
| 4-2 | 23.8 |
| 4-3 | 14.0 |
| 4-4 | 8.4 |
| 4-5 | 9.1 |

### AsPC-1 KRAS G12D Cell Viability Assay

AsPC-1 KRAS(G12D)-HiBit cells (Promega product #CS3023190) were maintained in growth medium containing RPMI 1640 medium, no phenol red (Gibco product #11835030) supplemented with 1X penicillin/streptomycin/Glutamine (Corning product #30-009-CI) and 10% fetal bovine serum (HyClone product #SH30396.03). Cells were passaged 2 times per week to maintain sub-confluent densities. Passages 5 to 12 were used for assays. Test compounds were prepared in 100% DMSO in 384-well polypropylene plates (Greiner Bio-One, product #784201) with 8 compounds per plate in grouped replicates of 4 at 10 serially diluted concentrations (1:3). AsPC-1 KRAS-HiBit cells were harvested and suspended in growth medium (described above). The cells were seeded in 384-well black clear round bottom ultra-low attachment spheroid microplate (Corning, product #3830) at 1,000 cells per well in 80 μL. The plates containing cells were incubated at 37° C. with 5% CO₂ for 72 hours to allow spheroid formation, prior to dosing with 0.4 μL of test compounds using a Biomek 17. The cells treated with compounds were incubated at 37° C., 5% CO₂ for another 96 hours. The cells were then equilibrated to room temperature for 30 minutes and CellTiter-Glo® 3D Cell Viability reagent (Promega, product #G9683) was prepared according to manufacturer protocol and equilibrated to room temperature. Medium was aspirated from assay plates until 40 μL remained using a BioTek EL406 washer dispenser (Agilent), and 40 μL of CellTiter-Glo® 3D Cell Viability reagent was added per well. The plates were placed on a Biomek 17, where mixing was performed by aspirating and dispensing 50 μL 15 times across the entire plate to ensure thorough homogenization. The plates were then incubated at room temperature for an additional 5 minutes before luminescence was measured using an EnVision multimode microplate reader (Revvity). The luminescence signal was normalized to negative (DMSO, 100%) and positive (10 μM puromycin, 0%) controls and plotted against log 10 concentration of test compounds. The data was fitted to a 4 variables non-linear regression analysis to determine EC₅₀. EC₅₀ is defined as the concentration at which the mid-point between the top and bottom plateaus is reached.

TABLE 7
Biological data

| Example | EC₅₀ (nM) |
|---|---|
| 1-33 | 1.6 |
| 1-34 | 1.5 |
| 1-35 | 0.6 |
| 1-36 | 1.5 |
| 1-37 | 1.5 |
| 1-38 | 1.1 |
| 1-39 | 1.8 |
| 1-40 | 4.3 |
| 1-42 | 1.0 |
| 1-43 | 12.1 |
| 1-44 | 5.5 |
| 1-45 | 21.8 |
| 1-46 | 3.8 |
| 1-47 | 1.3 |
| 1-48 | 3.2 |
| 2-30 | 24.9 |
| 2-33 | 2.9 |
| 2-34 | 4.5 |
| 2-35 | 2.2 |
| 2-37 | 2.4 |
| 2-38 | 9.2 |
| 2-43 | 1.4 |
| 2-44 | 2.0 |
| 2-45 | 1.9 |
| 2-49 | 2.7 |
| 2-50 | 4.4 |
| 2-68 | 2.3 |
| 2-70 | 2.3 |
| 2-71 | 5.0 |
| 2-72 | 4.6 |
| 2-76 | 25.2 |
| 2-77 | 17.7 |
| 4-6 | 3.2 |
| 5-1 | 1.8 |
| 5-2 | 3.5 |
| 5-3 | 1.0 |
| 5-4 | 7.0 |
| 5-8 | 15.5 |
| 5-9 | 13.0 |
| 5-10 | 5.0 |
| 6-1 | 1.2 |
| 6-2 | 120 |
| 6-3 | 2.8 |
| 6-4 | 2.6 |

The present disclosure provides reference to various embodiments and techniques. However, it should be understood that many variations and modifications can be made while remaining within the spirit and scope of the present disclosure. The description is made with the understanding that it is to be considered an exemplification of the claimed subject matter, and is not intended to limit the appended claims to the specific embodiments illustrated.

1. A compound of Formula (I): 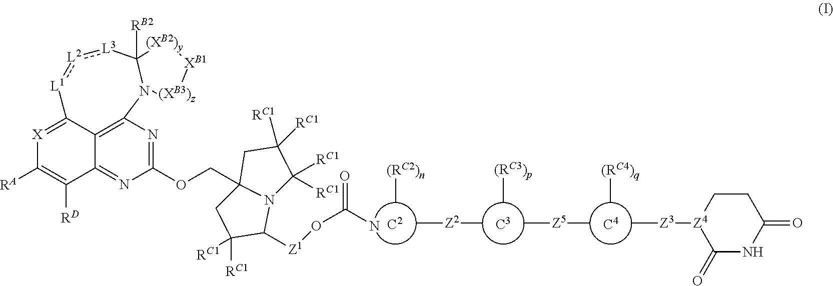
or a pharmaceutically acceptable salt thereof,
wherein
each R^{C1} is independently H, C₁-C₆ alkyl, C₂-C₆ alkoxyalkyl, C₁-C₆ hydroxyalkyl, halo, C₁-C₆ haloalkyl, C₁-C₆ haloalkoxy, —OH, —CN, C₁-C₆ cyanoalkyl, C₃-C₆ cycloalkyl, ═CH₂, ═CHF, or ═CF₂, or
alternatively, two R^{C1} attached to the same carbon form a C₃-C₆-membered cycloalkyl or 4- to 6-membered heterocyclyl, wherein the cycloalkyl and heterocyclyl is substituted by 0, 1, or 2 halo;
Z¹ is —(CR^{2f}R^{2g})ₘ—;
R^{2f} and R^{2g} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2f} f and R^{2g} can combine with the carbon to which they are attached to form a C₃-C₆ cycloalkyl;
m is 1, 2, or 3;
ring C2 is 4- to 16-membered heterocyclyl having at least one ring nitrogen;
each R^{C2} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, —CN, C₁-C₆ cyanoalkyl, or C₃-C₆-cycloalkyl;
n is 0, 1, 2, 3, or 4;
Z² is C₁-C₃ alkyl, C₂-C₃ alkenyl, C₂-C₃ alkynyl, C₃-C4 cycloalkyl, —O—C₁-C₃ alkyl, —O—C₂-C₃ alkenyl, —O—C₂-C₃ alkynyl, —O—, —S—, —NR^{Z2b}—, —C(O)—, —S(O)—, S(O)₂—, S(O)(═NR^{Z2})—, oxetanyl, or a bond; wherein the alkyl and cycloalkyl is substituted with 0, 1, 2, 3, or 4 Z^{2a}; wherein the alkenyl is substituted with 0, 1, or 2 Z^{2c};
each Z^{2a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆thiohaloalkyl, —OH, —CN, C₁-C₆ cyanoalkyl, C₃-C₆ cycloalkyl, or 4- to 10-membered heterocyclyl;
each Z^{2c} is independently C₁-C₆ alkyl, halo, C₁-C₆ haloalkyl, or C₃-C₆ cycloalkyl;
R^{Z2} is H, C₁-C₆ alkyl, C₁-C₆ haloalkyl, or C₃-C₆ cycloalkyl;
R^{Z2b} is H, C₁-C₆ alkyl, C₁-C₆ haloalkyl, or C₃-C₆ cycloalkyl;
ring C3 is C₃-C₈ cycloalkyl, 4- to 16-membered heterocyclyl, C₆-C₁₀ aryl, 5- to 10-membered heteroaryl, or absent;
each R^{C3} is independently C₁-C₆ alkyl, C₂-C₆ alkenyl, C₂-C₆ alkynyl, halo, C₁-C₆ haloalkyl, C₁-C₆ haloalkoxy, C₁-C₆ thioalkyl, C₁-C₆ thiohaloalkyl, —OH, oxo, —CN, C₁-C₆ alkoxy, C₂-C₆ alkoxyalkyl, C₁-C₆ hydroxyalkyl, C₁-C₆ cyanoalkyl, or C₃-C₆-cycloalkyl;
p is 0, 1, 2, 3, or 4;
Z⁵ is C₁-C₃ alkyl, C₂-C₃ alkenyl, C₂-C₃ alkynyl, C₃-C₄ cycloalkyl, —O—C₁-C₃ alkyl, —O—C₂-C₃ alkenyl, —O—C₂-C₃ alkynyl, —O—, —S—, —NR^{Z5b}—, —C(O)—, —S(O)—, S(O)₂—, S(O)(═NR^{Z5})—, or a bond; wherein the alkyl and cycloalkyl is substituted with 0, 1, 2, 3, or 4 Z^{5a};
each Z^{5a} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆thiohaloalkyl, —OH, —CN, C₁-C₆ cyanoalkyl, C₃-C₆ cycloalkyl, or 4- to 10-membered heterocyclyl;
R^{Z5} is H, C₁-C₆ alkyl, C₁-C₆ haloalkyl or C₃-C₆ cycloalkyl;
R^{Z5b} is H, C₁-C₆ alkyl, C₁-C₆ haloalkyl, or C₃-C₆ cycloalkyl;
ring C4 is 5 to 14-membered heterocyclyl, C₆-C₁₀ aryl, or 5- to 10-membered heteroaryl;
each R^{C4} is independently C₁-C₆ alkyl, C₂-C₆ alkenyl, C₂-C₆ alkynyl, C₁-C₆ alkoxy, C₂-C₆ alkoxyalkyl, C₁-C₆ hydroxyalkyl, halo, C₁-C₆ haloalkyl, C₁-C₆ haloalkoxy, C₁-C₆ thioalkyl, C₁-C₆ thiohaloalkyl, —OH, oxo, —CN, C₁-C₃ cyanoalkyl, C₃-C₆ cycloalkyl, or 4- to 10-membered heterocyclyl;
q is 0, 1, 2, 3, or 4;
Z³ is —NH—, —C(O)NH—, or a bond, and Z⁴ is —CH—;
alternatively, Z³ is a bond and Z⁴ is N;
X is N, CH, or CR^{x};
R^{x} is halo, C₁-C₃ haloalkyl, or C₁-C₃ cyanoalkyl;
L¹ is O, CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6-membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, or C₁-C₆ haloalkyl;
R^{1e} is H, C₁-C₃ alkyl, halo, C₁-C₆ haloalkyl, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is a bond or CR^{2a}R^{2b}, C(═CR^{2c}R^{2d}), C(═O), ═C(R^{2e})—, O, or S, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, or C₁-C₆ haloalkyl;
R^{2e} is H, C₁-C₃ alkyl, halo, C₁-C₆ haloalkyl, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
L³ is a bond, CR^{3a}R^{3b}, C(═CR^{3c}R^{3d}), C(═O), or ═C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, or C₁-C₆ haloalkyl;
R^{3e} is H, C₁-C₃ alkyl, halo, C₁-C₆ haloalkyl, C₁-C₃ cyanoalkyl, or C₃-C₆ cycloalkyl;
alternatively, R^{2e} and R^{3e} can combine with the atoms to which they are attached to form a C5-C₆ cycloalkyl, 5- to 10-membered heterocyclyl, C₆-C₁₀ aryl, or 5- to 14-membered heteroaryl;
such that when L² is ═C(R^{2e})— then L¹ is —C(R^{1e})═ or L³ is ═C(R^{3e})—, and when L³ is ═C(R^{3e})— then L² is ═C(R^{2e})—;
R^{A} is phenyl, naphthyl, or 5- to 14-membered heteroaryl, wherein R^{A} is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C1o alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆haloalkyl, —CN, —C(O)R^{A2a}, —C(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —SRA2a, —S(O)₂R^{A2a}, —S(O)₂OR^{A2a}, —N(R^{A2a})(R^{A2b}), —(C₀-C₃ alkyl)-SF₅, C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, —OR^{A3a}, —SR^{A3a}, —N(R^{A3}a)(R^{A3}b), C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ thiohaloalkyl, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C1o aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently H, C₁-C₁₀ alkyl, C₂-C₁₀ alkenyl, C₂-C₁₀ alkynyl, halo, C₁-C₆ haloalkyl, —CN, or C₃-C₈ cycloalkyl;
X^{B1} is C(R^{B1})(R^{B1}), O, S or Si(R^{B1})(R^{B1});
each X^{B2} and X^{B3} is independently C(R^{B1})(R^{B});
each R^{B1} is independently hydrogen, halo, —CN, —OH, C₁-C₆ alkyl, C₁-C₆ haloalkyl, C₁-C₆ hydroxyalkyl, C₁-C₆ cyanoalkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, 2, or 3 R^{B3};
alternatively, two R^{B1} attached to two different atoms can combine to form a C₃-C₁₀ cycloalkyl or 4- to 10-membered heterocyclyl, wherein the cycloalkyl and heterocyclyl are substituted with 0, 1, 2, or 3 R^{B3};
alternatively, two R^{B1} attached to the same atom can combine to form a C₃-C₁₀ cycloalkyl or 4- to 10-membered heterocyclyl, wherein the cycloalkyl and heterocyclyl are substituted with 0, 1, 2, or 3 R^{B3};
each y and z is independently 1, 2, 3, or 4;
R^{B2} is H or C₁-C₆ alkyl;
alternatively, R^{B2} can combine with R^{B1} on an adjacent atom to form a C₃-C₁₀ cycloalkyl or 4- to 10-membered heterocyclyl, wherein the cycloalkyl and heterocyclyl are substituted with 0, 1, 2, or 3 R^{B3};
each R^{B3} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
R^{D} is halo;
wherein, unless otherwise indicated, each heterocyclyl has 1, 2, 3, or 4 heteroatoms selected from N, O, S, and Si; and
each heteroaryl has 1, 2, 3, or 4 heteroatoms selected from N, O, and S.

2. The compound of claim 1, or pharmaceutically acceptable salt thereof, having the structure of Formula (Ia): 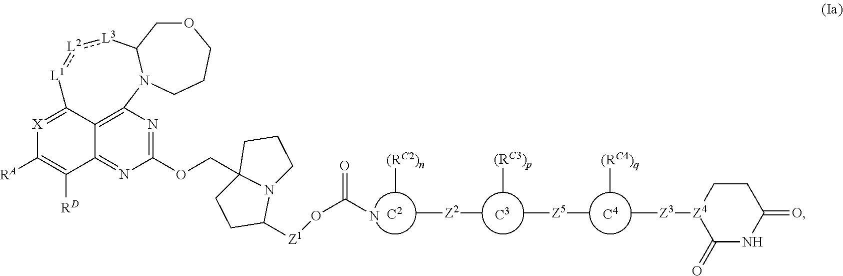
wherein:
X is N;
L³ is a bond; and
R^{D} is F.

3.-4. (canceled)

5. The compound of claim 1, or pharmaceutically acceptable salt thereof, wherein:
L¹ is CR^{1a}R^{1b}; and
L² is CHR^{2b}, wherein R^{2b} is H.

6.-9. (canceled)

10. The compound of claim 1, or pharmaceutically acceptable salt thereof, wherein
L¹ is —C(R^{1e})═; and
L² is ═C(R^{2e})—.

11.-13. (canceled)

14. The compound of claim 1, or a pharmaceutically acceptable salt thereof, wherein
R^{A} is phenyl substituted with 3 R^{A2}, or
R^{A} is napthyl substituted with 0, 1, 2, 3, 4, or 5 R^{A2}.

15.-23. (canceled)

24. The compound of claim 1, or a pharmaceutically acceptable salt thereof, wherein R^{A} is

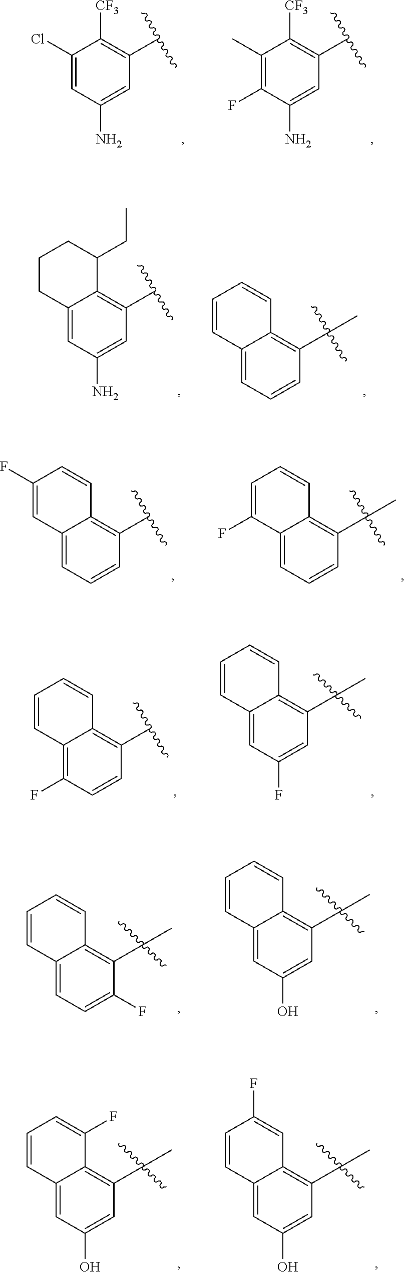

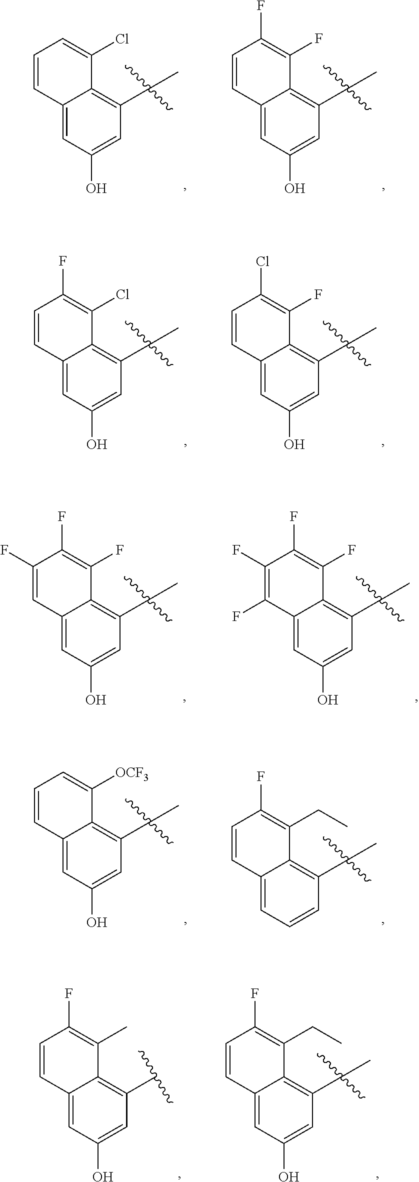

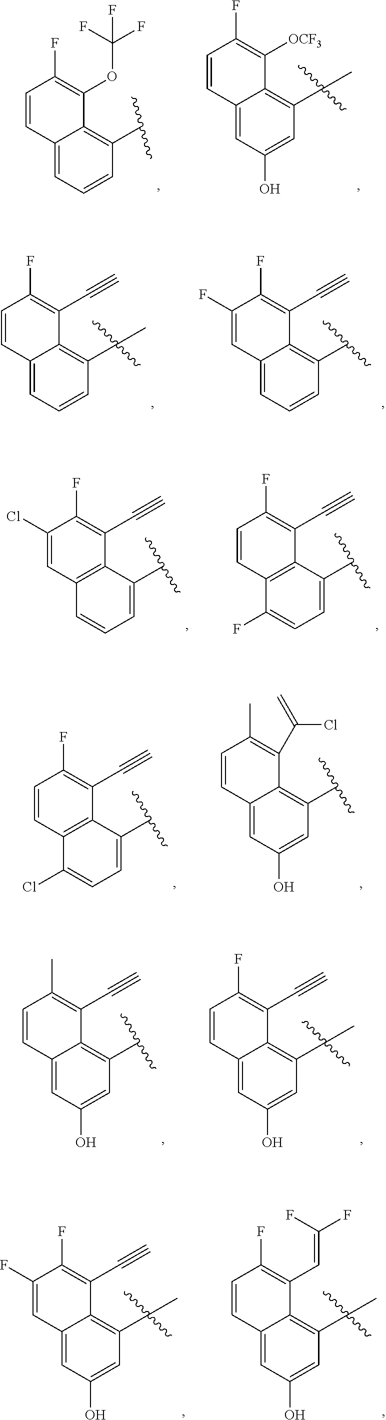

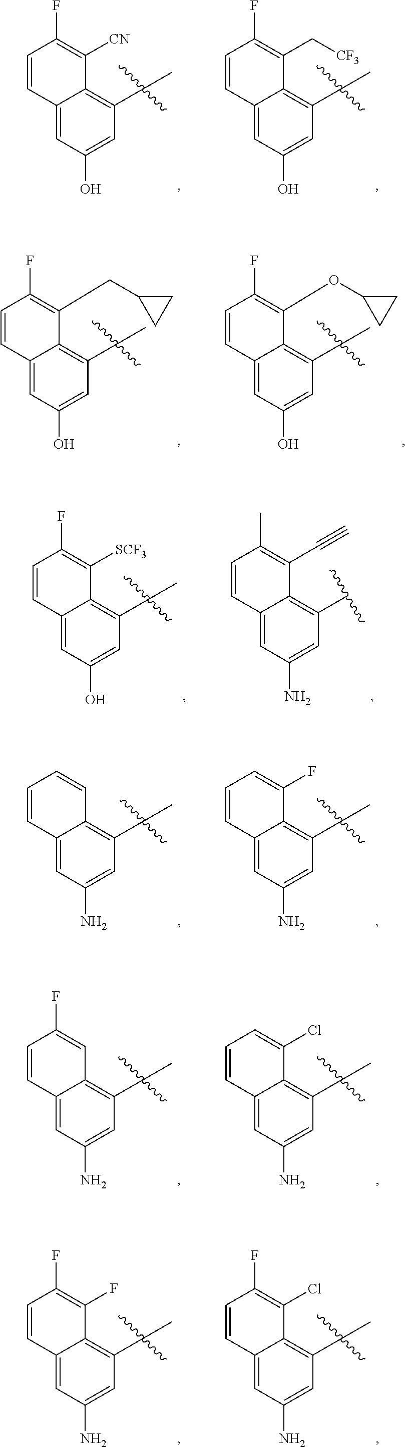

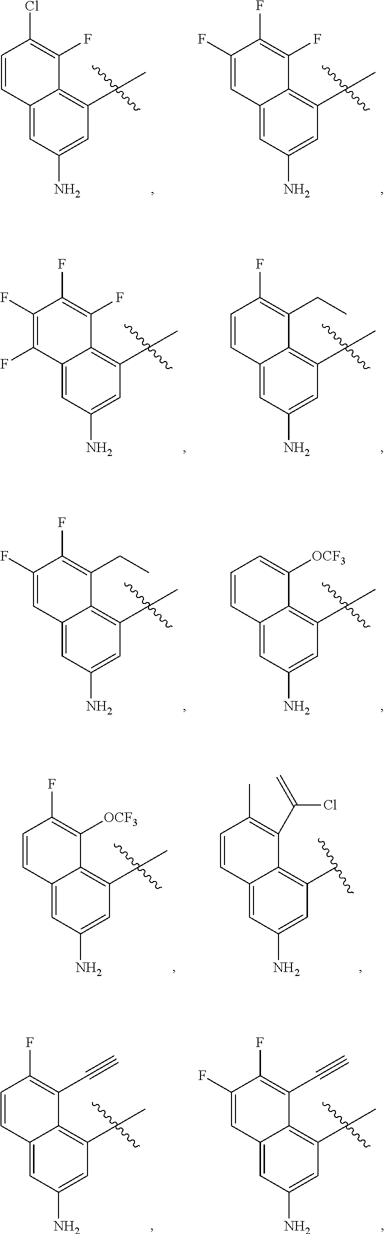

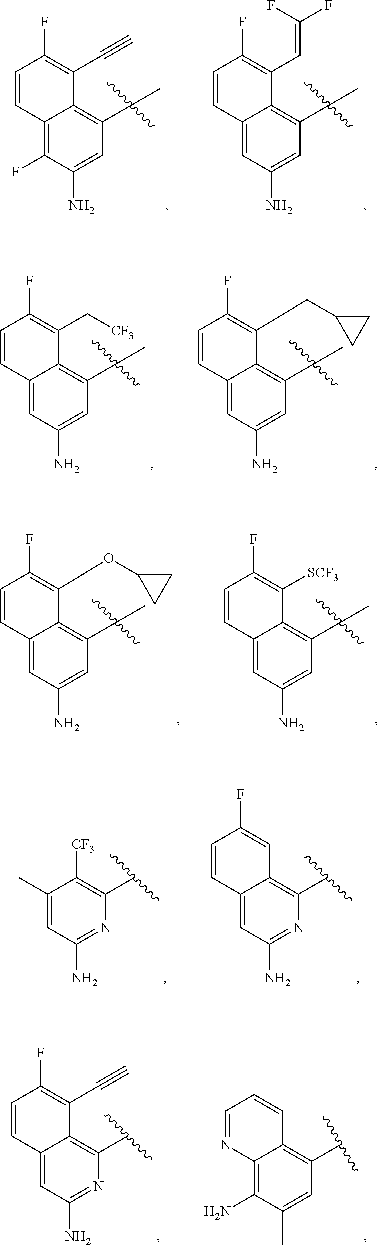

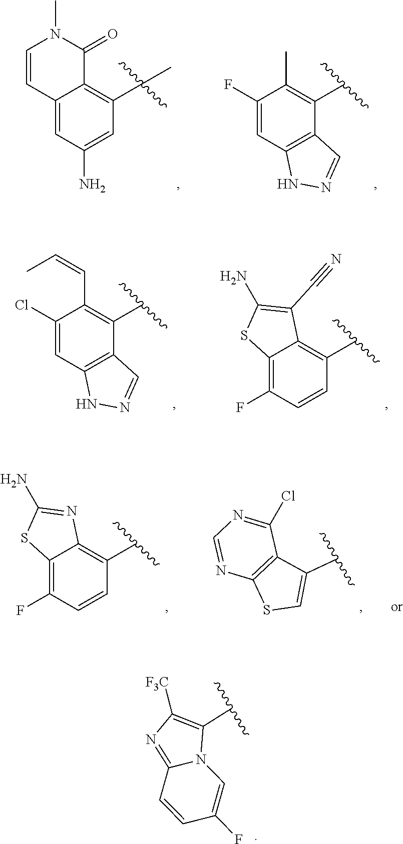

25. (canceled)

26. The compound of claim 1, or pharmaceutically acceptable salt thereof, wherein Z¹ is —CH₂—.

27. The compound of claim 1, or pharmaceutically acceptable salt thereof, wherein
ring C2 is 6- to 14-membered heterocyclyl having at least one ring nitrogen, wherein the heterocyclyl has 1, 2, or 3 additional heteroatoms selected from N, O, and S;
each R^{C2} is independently C₁-C₆ alkyl, halo, or —OH; and
n is 0, 1, or 2.

28.-30. (canceled)

31. The compound of claim 1, or pharmaceutically acceptable salt thereof, wherein Z² is C₁-C₃ alkyl, C₁-C₃ alkyl substituted with 1 or 2 halo, C₂-C₃ alkynyl, —O—C₁-C₃ alkyl, —O—C₂-C₃ alkynyl, —O—, or a bond.

32.-34. (canceled)

35. The compound of claim 1, or pharmaceutically acceptable salt thereof, wherein
ring C3 is C₃-C₈ cycloalkyl, 4- to 16-membered heterocyclyl, 5- to 10-membered heteroaryl, or absent, wherein the heterocyclyl and heteroaryl have 1, 2, 3, or 4 heteroatoms selected from N, O, and S;
each R^{C3} is independently C₁-C₆ alkyl or halo; and
p is 0, 1, or 2.

36.-39. (canceled)

40. The compound of claim 1, or pharmaceutically acceptable salt thereof, wherein Z⁵ is a bond.

41. The compound of claim 1, or pharmaceutically acceptable salt thereof, wherein
ring C4 is 5 to 14-membered heterocyclyl, C₆-C₁₀ aryl or 5- to 10-membered heteroaryl, wherein the heterocyclyl and the heteroaryl each has 1, 2, 3, or 4 heteroatoms selected from N, O, and S;
each R^{C4} is independently C₁-C₆ alkyl, halo, oxo, or C₃-C₆ cycloalkyl; and
q is 0, 1, 2, or 3.

42.-44. (canceled)

45. The compound of claim 1, or pharmaceutically acceptable salt thereof, wherein Z³ is a bond.

46.-47. (canceled)

48. The compound of claim 1, or pharmaceutically acceptable salt thereof, wherein:
X is N;
L¹ is CR^{1a}R^{1b} or —C(R^{1e})═;
R^{1a} and R^{1b} are each independently H, C₁-C₃ alkyl, or halo;
R^{1e} is H or C₁-C₃ alkyl;
L² is CHR^{2b} or ═C(R^{2e})—;
R^{2b} is H, C₁-C₃ alkyl, or halo;
R^{2e} is H or C₁-C₃ alkyl;
L³ is a bond;
R^{A} is phenyl or naphthyl, wherein R^{A} is substituted with 0, 1, 2, or 3 R^{A2};
each R^{A2} is independently —OH, C₁-C₁₀ alkyl, C₂-C₆ alkynyl, C₁-C₁₀ alkoxy, halo, —N(R^{A2a})(R^{A2b}), wherein each alkyl, alkynyl, and alkoxy is substituted with 0, 1, 2, or 3 R^{A3};
each R^{A2a} and R^{A2b} is independently H or C₁-C₁₀ alkyl;
each R^{A3} is independently halo;
alternatively, two R^{A2} can combine to form a C₃-C₁₀ cycloalkyl or C₆-C₁₀ aryl on two adjacent atoms on R^{A}, wherein each cycloalkyl and aryl is substituted with 0, 1, 2, or 3 R^{A5};
each R^{A5} is independently H, C₁-C₁₀ alkyl, C₂-C₁₀ alkynyl, halo, C₁-C₆ haloalkyl, or C₃-C₈ cycloalkyl;
R^{D} is F;
Z¹ is —CH₂—;
ring C2 is 6- to 14-membered heterocyclyl having at least one ring nitrogen, wherein the heterocyclyl has 1, 2, or 3 additional heteroatoms selected from N, O, and S;
each R^{C2} is independently C₁-C₆ alkyl, halo, or —OH;
n is 0, 1, or 2;
Z² is C₁-C₃ alkyl, C₁-C₃ alkyl substituted with 1 or 2 halo, C₂-C₃ alkynyl, —O—C₁-C₃ alkyl, —O—C₂-C₃ alkynyl, —O—, or a bond;
ring C3 is C₃-C₈ cycloalkyl, 4- to 16-membered heterocyclyl, 5- to 10-membered heteroaryl, or absent, wherein the heterocyclyl and heteroaryl have 1, 2, 3, or 4 heteroatoms selected from N, O, and S;
each R^{C3} is independently C₁-C₆ alkyl or halo;
p is 0, 1, or 2;
Z⁵ is CH₂ or a bond;
ring C4 is 5 to 14-membered heterocyclyl, C₆-C₁₀ aryl or 5- to 10-membered heteroaryl, wherein the heterocyclyl and the heteroaryl each has 1, 2, 3, or 4 heteroatoms selected from N, O, and S;
each R^{C4} is independently C₁-C₆ alkyl, halo, oxo, or C₃-C₆ cycloalkyl; and
q is 0, 1, 2, or 3;
Z³ is a bond; and
Z⁴ is —CH— or N.

49. The compound of claim 1, or pharmaceutically acceptable salt thereof, wherein 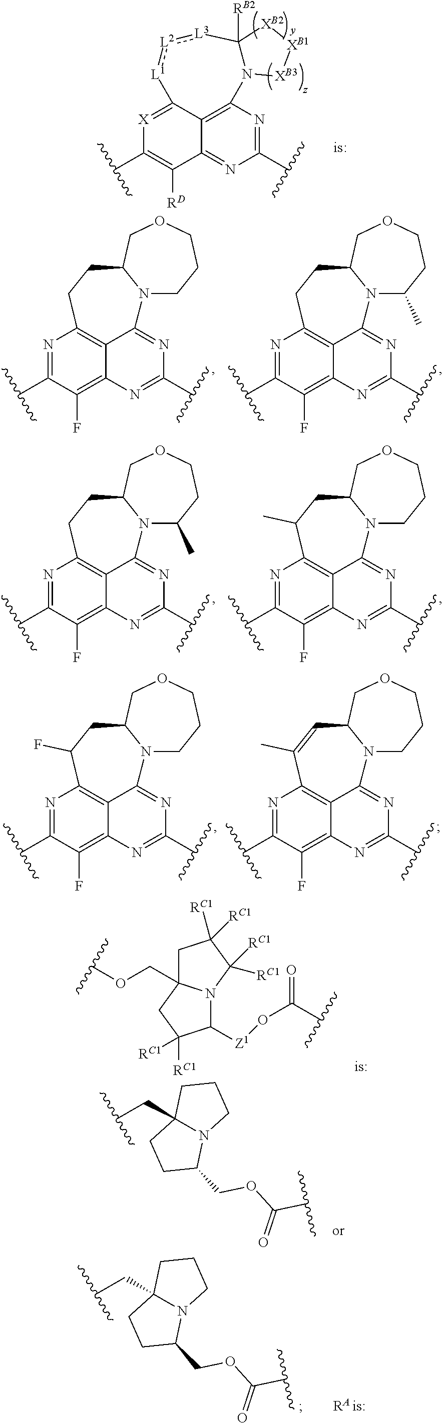 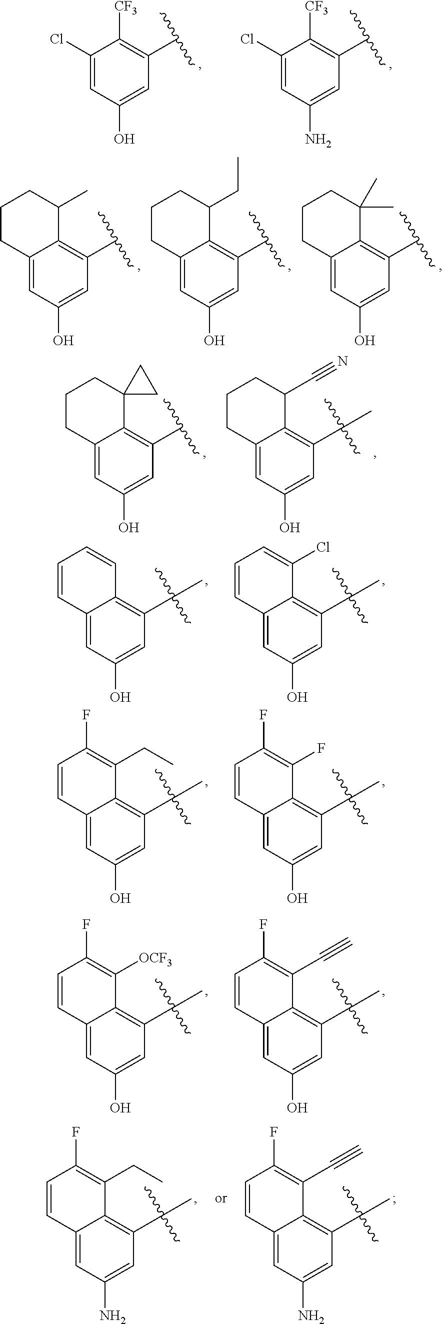 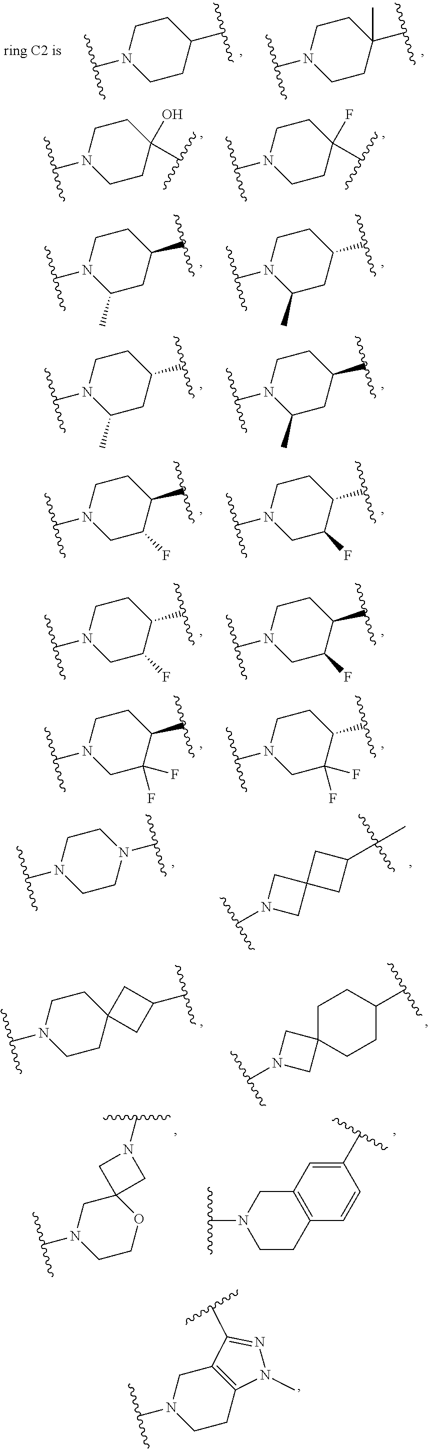 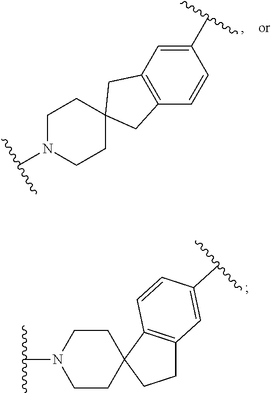 [SMILES: *c1ccc2c(c1)CC1(CCN(*)CC1)C2 ; *c1ccc2c(c1)CCC21CCN(*)CC1]
Z² is CH₂, CH(CH₃), C(CH₃)₂, —CH₂CH₂—, —CH₂CH₂CH₂—, CHF, CF₂, 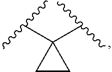 [SMILES: *C1(*)CC1]
—C≡C—, —OCH₂—, —CH₂O—, —OCH₂—C≡C—, —O—, or a bond;
ring C3 is absent or is: 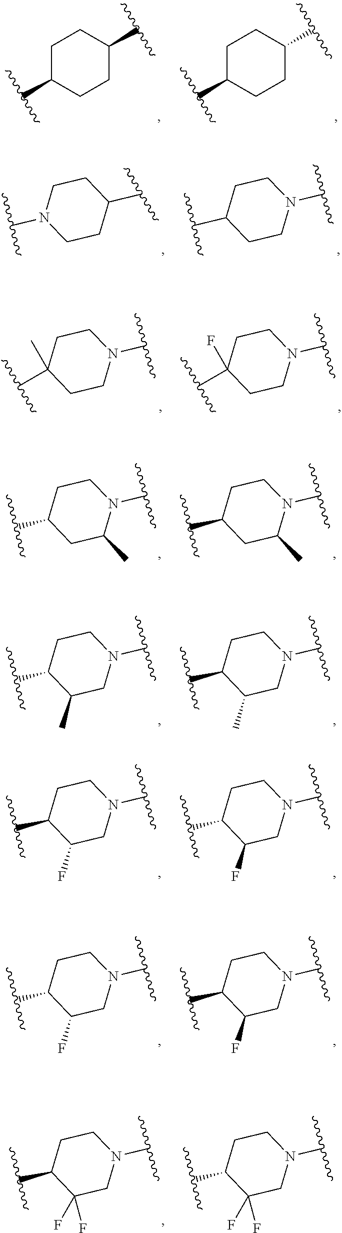 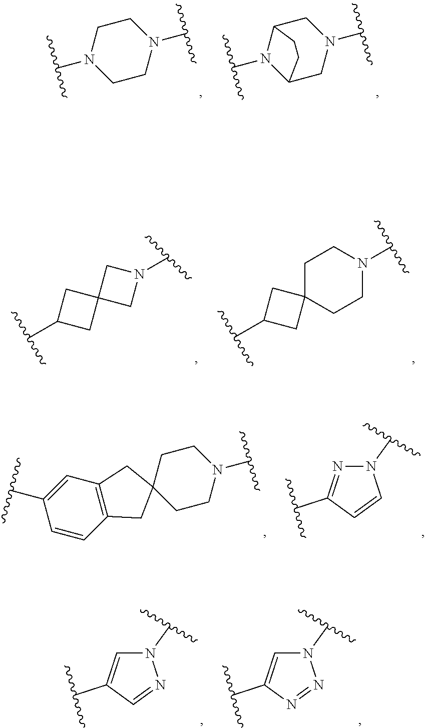
or absent;
Z⁵ is absent or CH₂;
ring C4 is 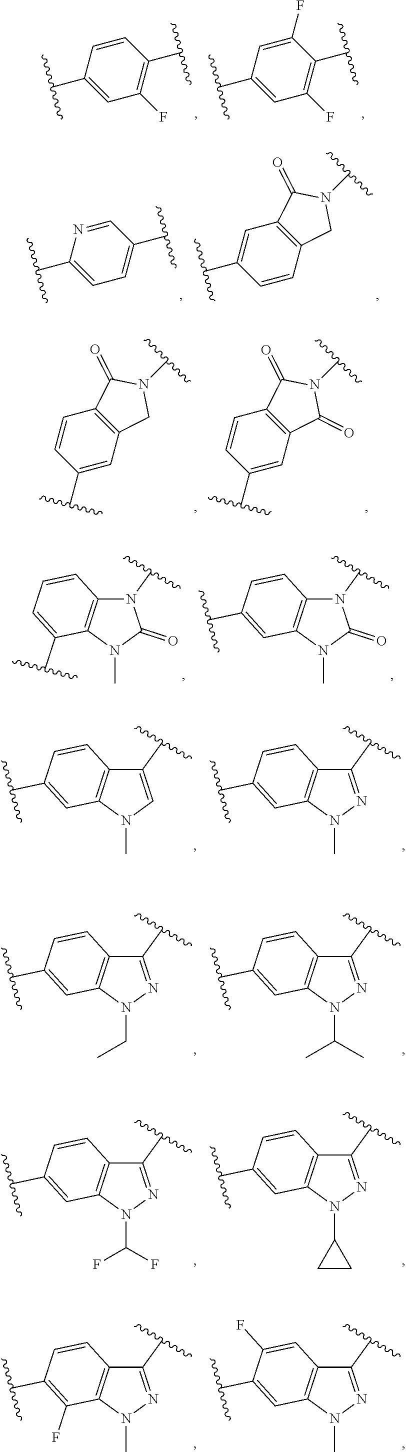 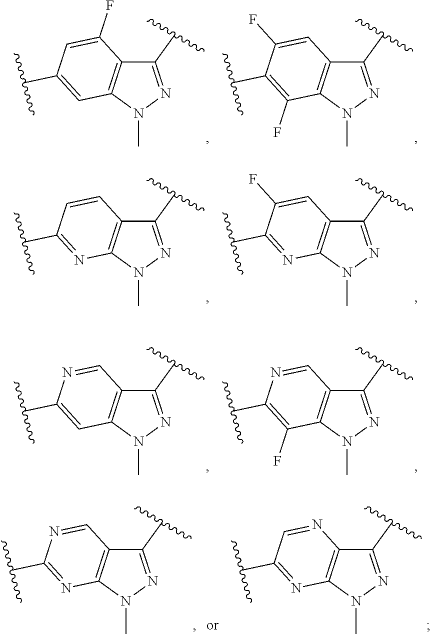
and
Z⁴ is —CH— or N.

50. The compound of claim 1, or pharmaceutically acceptable salt thereof, wherein the moiety 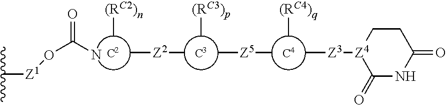
is

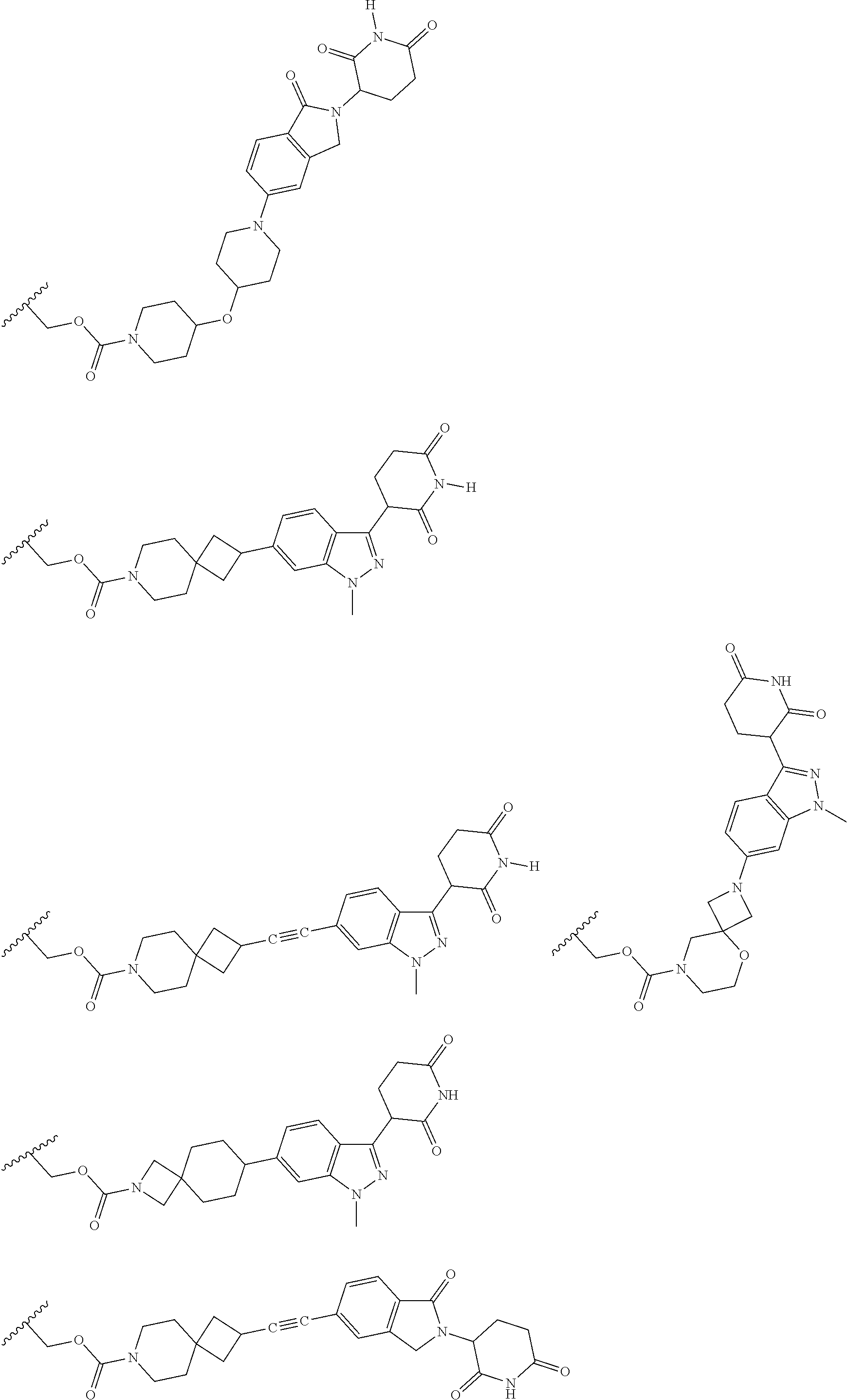 [SMILES: *COC(=O)N1CCC(OC2CCN(c3ccc4c(c3)CN(C3CCC(=O)NC3=O)C4=O)CC2)CC1 ; *COC(=O)N1CCC2(CC1)CC(c1ccc3c(C4CCC(=O)NC4=O)nn(C)c3c1)C2 ; *COC(=O)N1CCC2(CC1)CC(C#Cc1ccc3c(C4CCC(=O)NC4=O)nn(C)c3c1)C2 ; *COC(=O)N1CCOC2(C1)CN(c1ccc3c(C4CCC(=O)NC4=O)nn(C)c3c1)C2 ; *COC(=O)N1CC2(CCC(c3ccc4c(C5CCC(=O)NC5=O)nn(C)c4c3)CC2)C1 ; *COC(=O)N1CCC2(CC1)CC(C#Cc1ccc3c(c1)CN(C1CCC(=O)NC1=O)C3=O)C2]

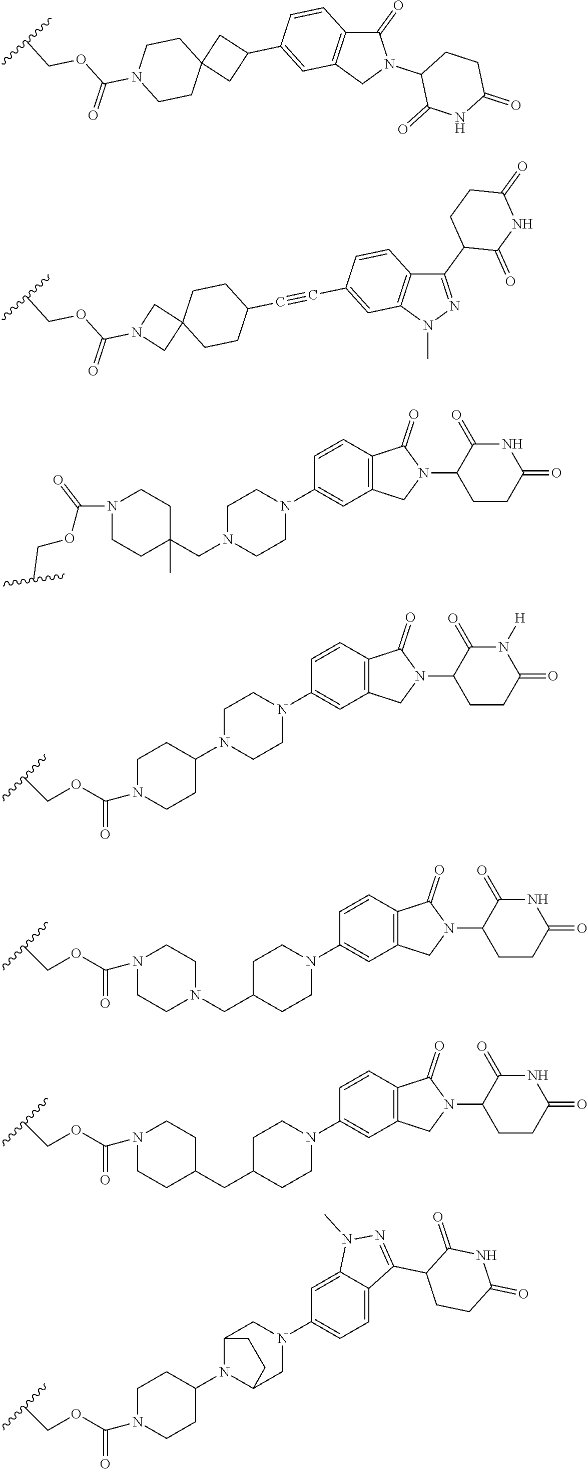

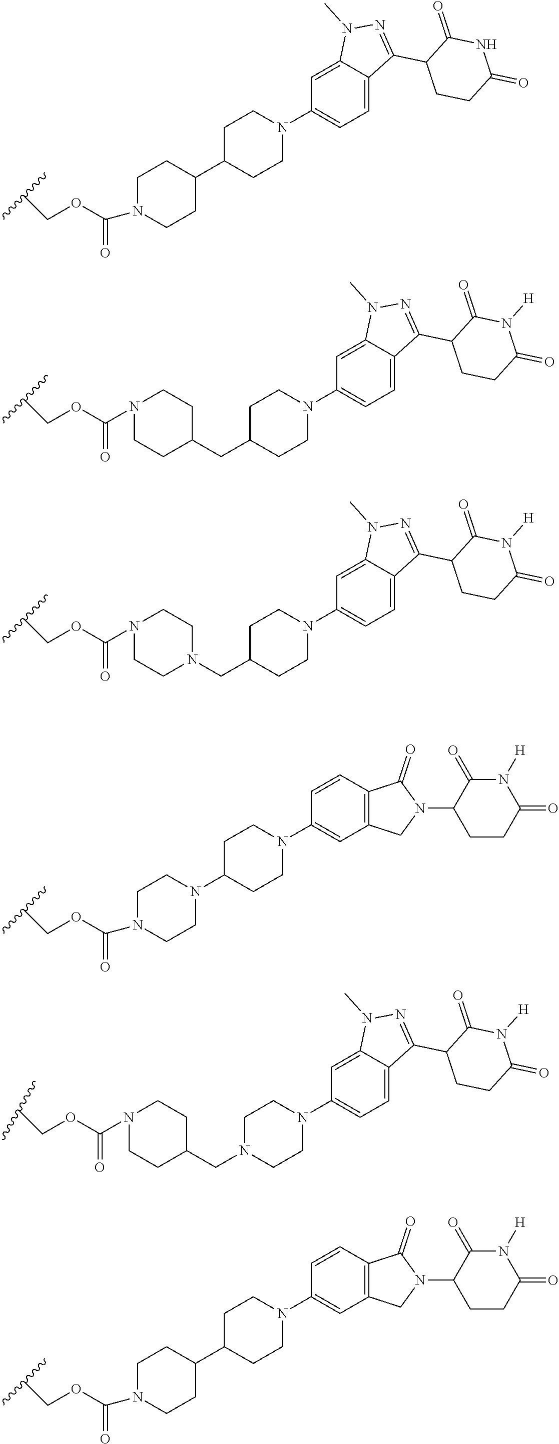 [SMILES: *COC(=O)N1CCC(C2CCN(c3ccc4c(C5CCC(=O)NC5=O)nn(C)c4c3)CC2)CC1 ; *COC(=O)N1CCC(CC2CCN(c3ccc4c(C5CCC(=O)NC5=O)nn(C)c4c3)CC2)CC1 ; *COC(=O)N1CCN(CC2CCN(c3ccc4c(C5CCC(=O)NC5=O)nn(C)c4c3)CC2)CC1 ; *COC(=O)N1CCN(C2CCN(c3ccc4c(c3)CN(C3CCC(=O)NC3=O)C4=O)CC2)CC1 ; *COC(=O)N1CCC(CN2CCN(c3ccc4c(C5CCC(=O)NC5=O)nn(C)c4c3)CC2)CC1 ; *COC(=O)N1CCC(C2CCN(c3ccc4c(c3)CN(C3CCC(=O)NC3=O)C4=O)CC2)CC1]

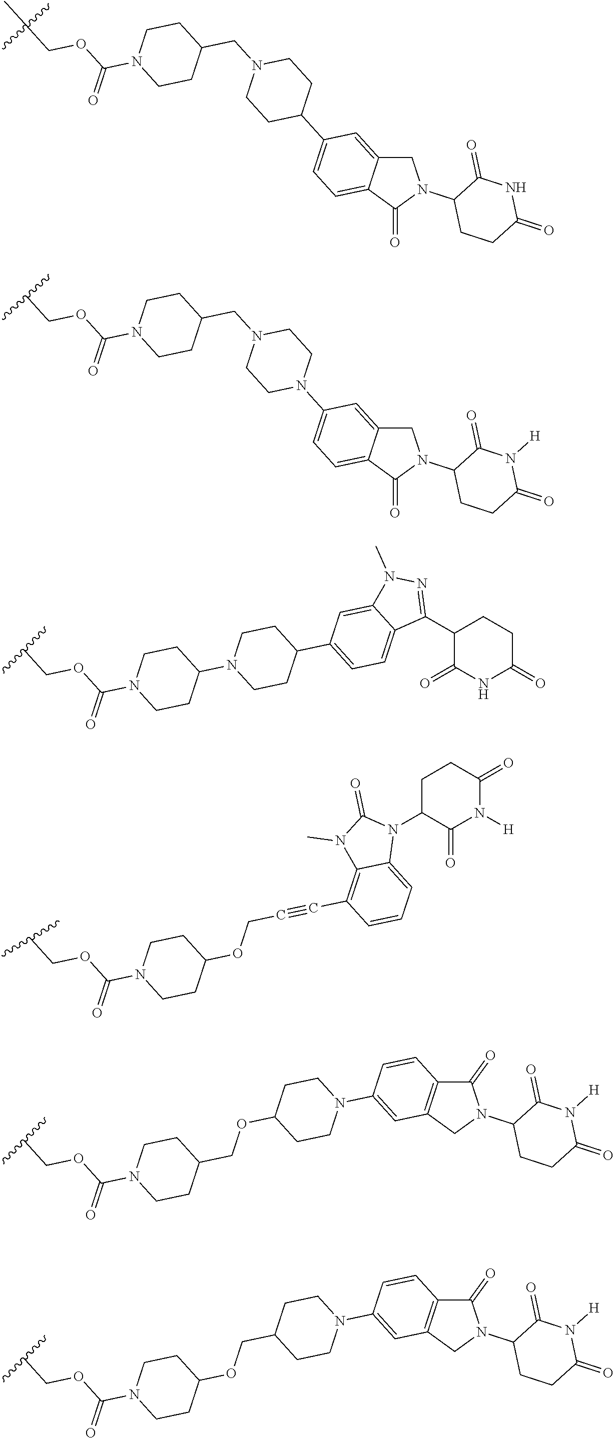

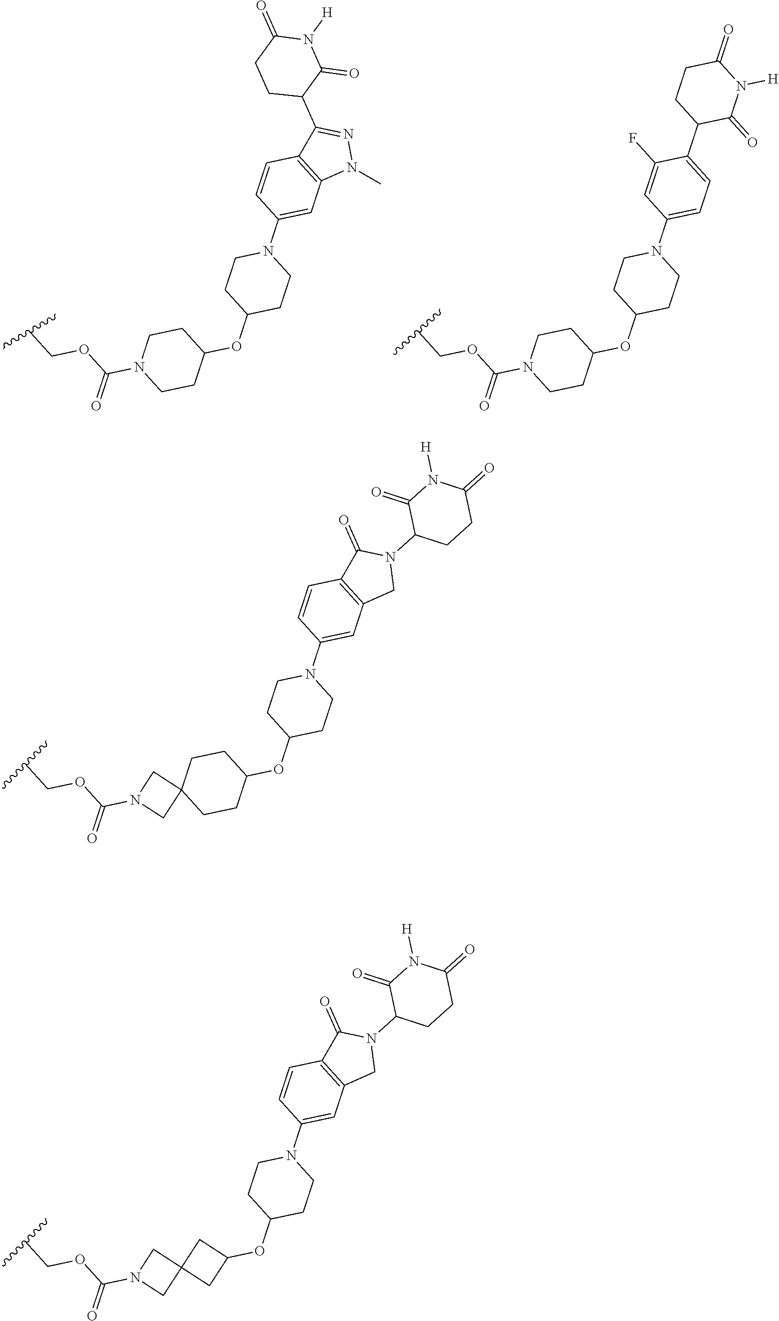 [SMILES: *COC(=O)N1CCC(OC2CCN(c3ccc4c(C5CCC(=O)NC5=O)nn(C)c4c3)CC2)CC1 ; *COC(=O)N1CCC(OC2CCN(c3ccc(C4CCC(=O)NC4=O)c(F)c3)CC2)CC1 ; *COC(=O)N1CC2(CCC(OC3CCN(c4ccc5c(c4)CN(C4CCC(=O)NC4=O)C5=O)CC3)CC2)C1 ; *COC(=O)N1CC2(CC(OC3CCN(c4ccc5c(c4)CN(C4CCC(=O)NC4=O)C5=O)CC3)C2)C1]

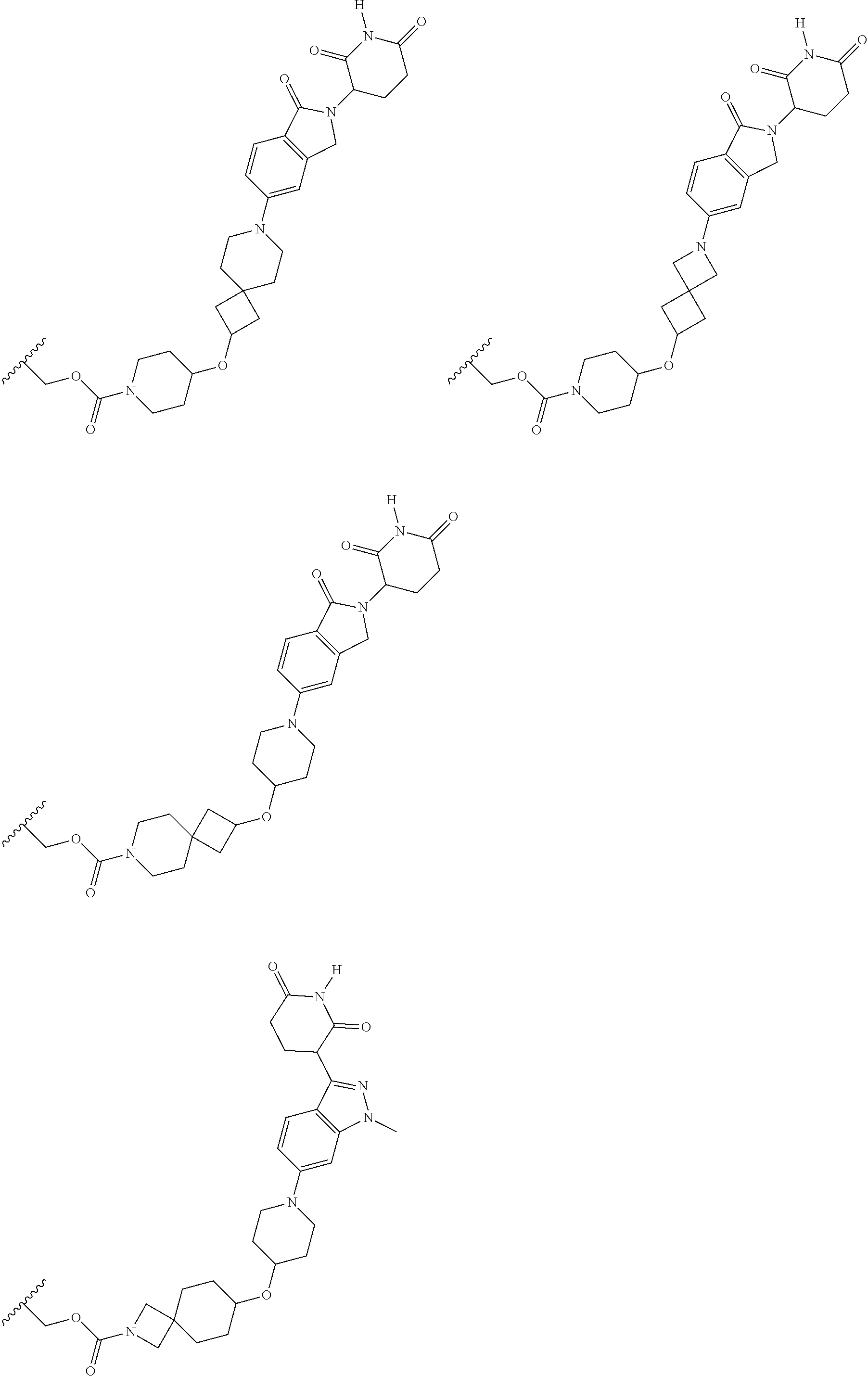 [SMILES: *COC(=O)N1CCC(OC2CC3(CCN(c4ccc5c(c4)CN(C4CCC(=O)NC4=O)C5=O)CC3)C2)CC1 ; *COC(=O)N1CCC(OC2CC3(C2)CN(c2ccc4c(c2)CN(C2CCC(=O)NC2=O)C4=O)C3)CC1 ; *COC(=O)N1CCC2(CC1)CC(OC1CCN(c3ccc4c(c3)CN(C3CCC(=O)NC3=O)C4=O)CC1)C2 ; *COC(=O)N1CC2(CCC(OC3CCN(c4ccc5c(C6CCC(=O)NC6=O)nn(C)c5c4)CC3)CC2)C1]

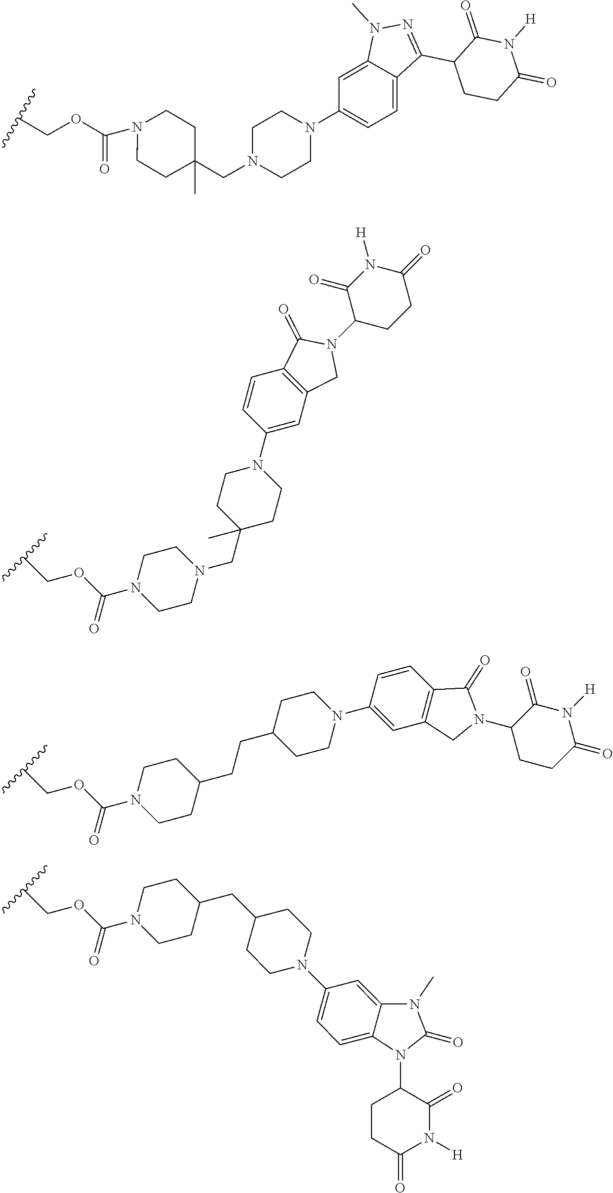 [SMILES: *COC(=O)N1CCC(C)(CN2CCN(c3ccc4c(C5CCC(=O)NC5=O)nn(C)c4c3)CC2)CC1 ; *COC(=O)N1CCN(CC2(C)CCN(c3ccc4c(c3)CN(C3CCC(=O)NC3=O)C4=O)CC2)CC1 ; *COC(=O)N1CCC(CCC2CCN(c3ccc4c(c3)CN(C3CCC(=O)NC3=O)C4=O)CC2)CC1 ; *COC(=O)N1CCC(CC2CCN(c3ccc4c(c3)n(C)c(=O)n4C3CCC(=O)NC3=O)CC2)CC1]

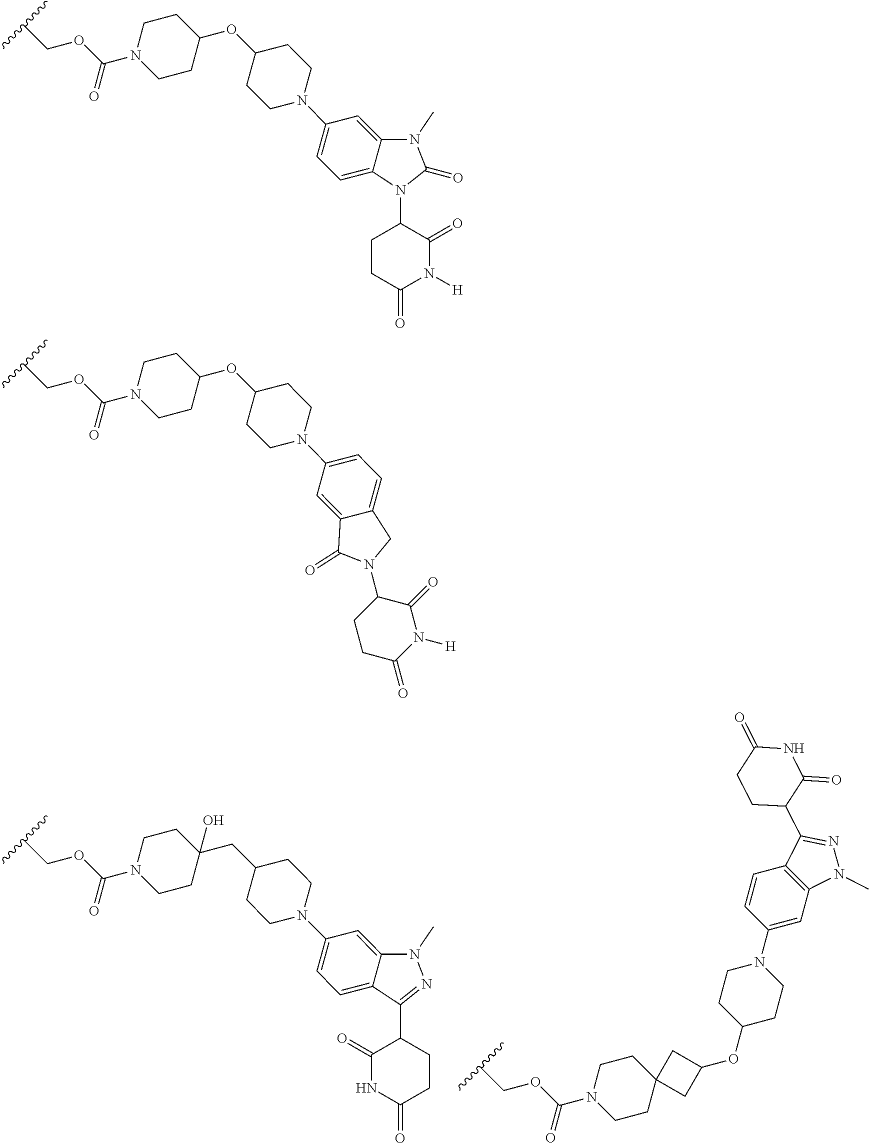 [SMILES: *COC(=O)N1CCC(OC2CCN(c3ccc4c(c3)n(C)c(=O)n4C3CCC(=O)NC3=O)CC2)CC1 ; *COC(=O)N1CCC(OC2CCN(c3ccc4c(c3)C(=O)N(C3CCC(=O)NC3=O)C4)CC2)CC1 ; *COC(=O)N1CCC(O)(CC2CCN(c3ccc4c(C5CCC(=O)NC5=O)nn(C)c4c3)CC2)CC1 ; *COC(=O)N1CCC2(CC1)CC(OC1CCN(c3ccc4c(C5CCC(=O)NC5=O)nn(C)c4c3)CC1)C2]

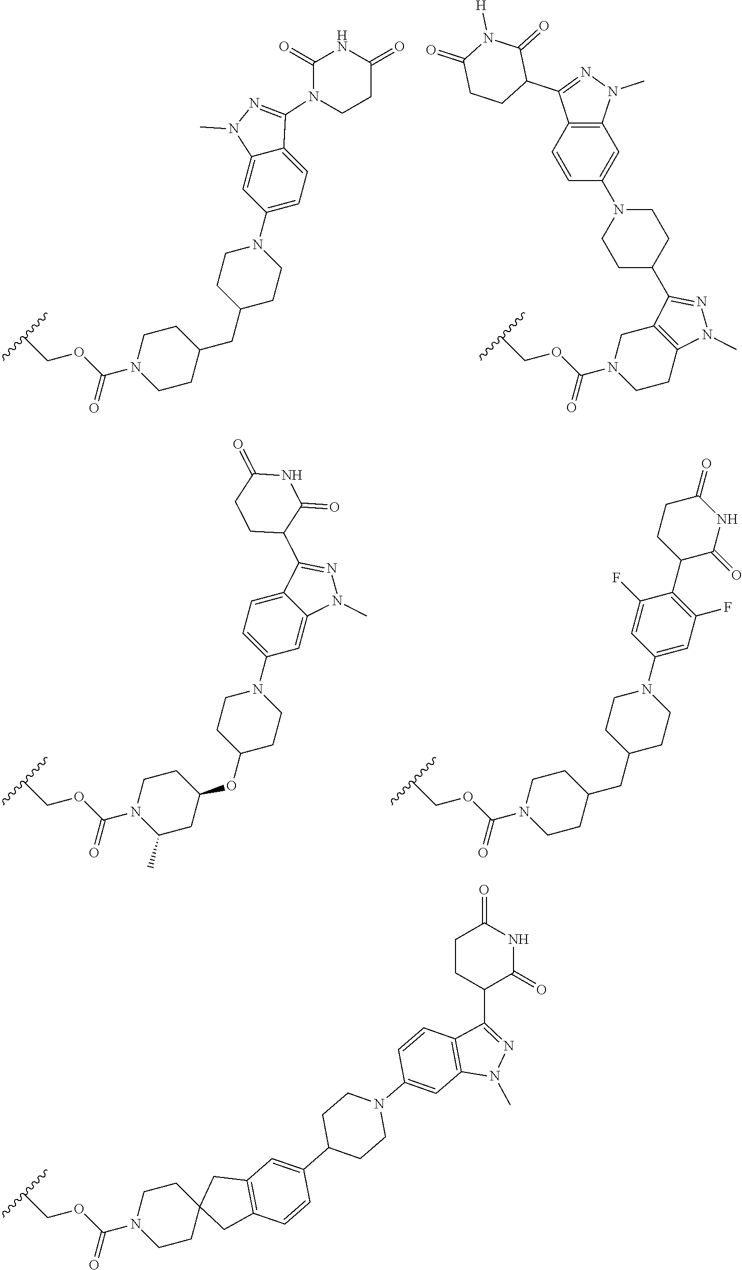

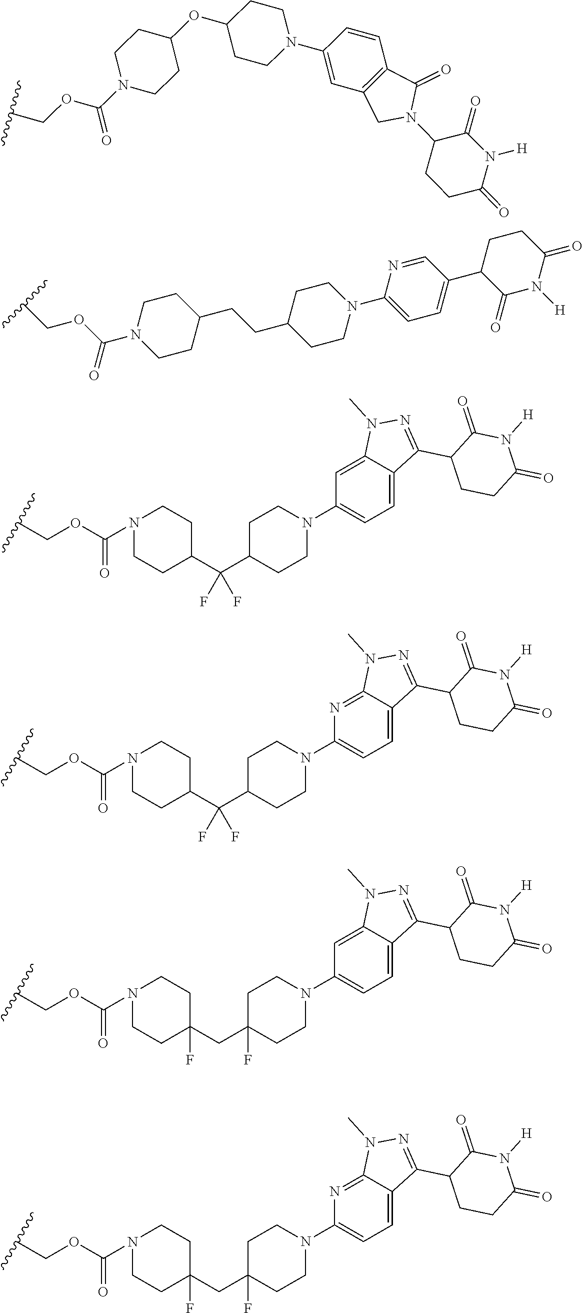

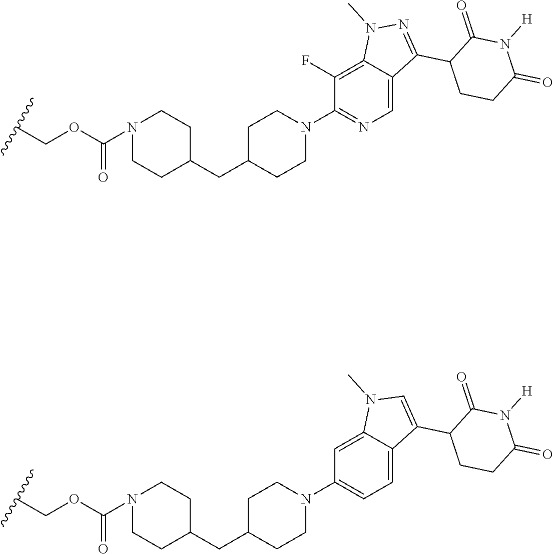 [SMILES: *COC(=O)N1CCC(CC2CCN(c3ncc4c(C5CCC(=O)NC5=O)nn(C)c4c3F)CC2)CC1 ; *COC(=O)N1CCC(CC2CCN(c3ccc4c(C5CCC(=O)NC5=O)cn(C)c4c3)CC2)CC1]

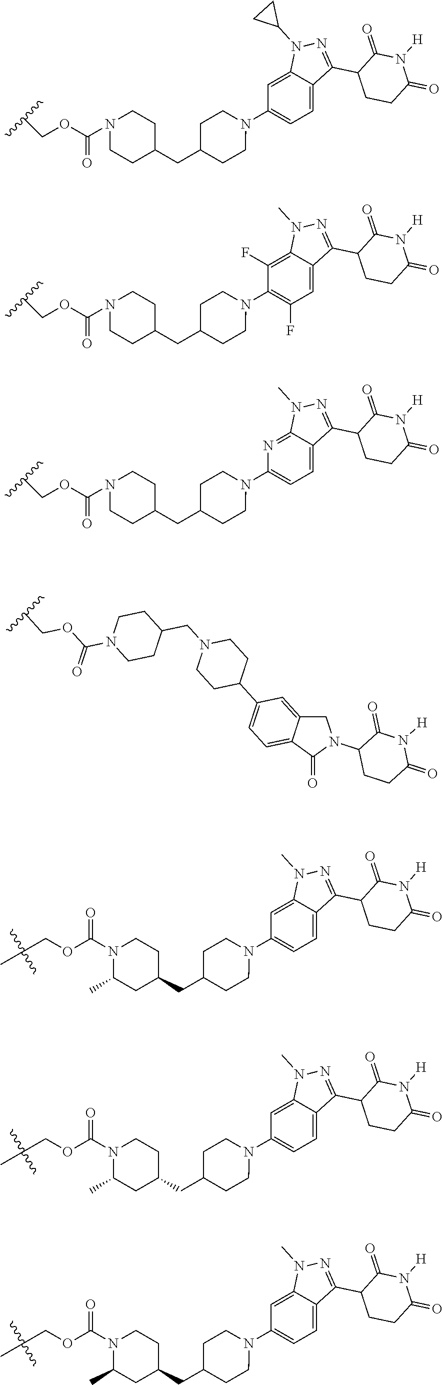

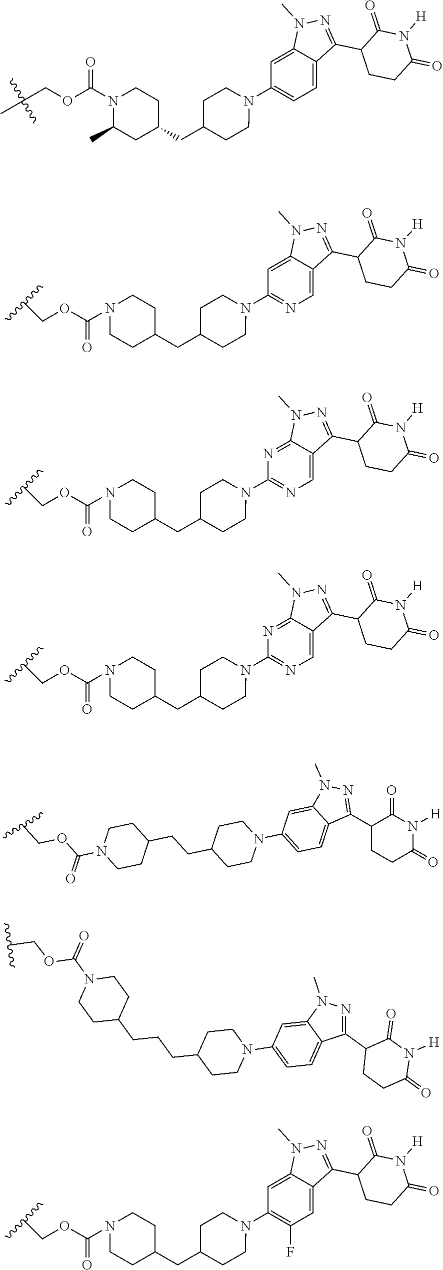

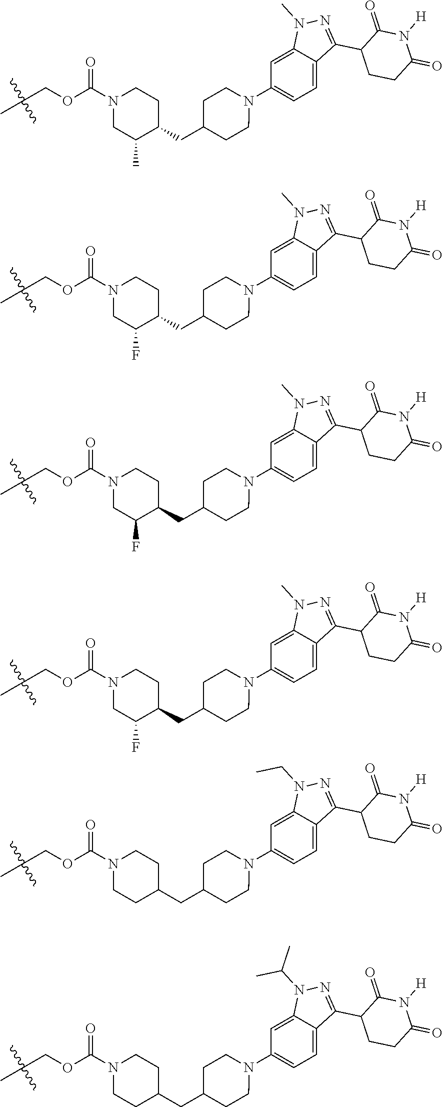 [SMILES: *COC(=O)N1CC[C@H](CC2CCN(c3ccc4c(C5CCC(=O)NC5=O)nn(C)c4c3)CC2)[C@H](C)C1 ; *COC(=O)N1CC[C@H](CC2CCN(c3ccc4c(C5CCC(=O)NC5=O)nn(C)c4c3)CC2)[C@H](F)C1 ; *COC(=O)N1CC[C@@H](CC2CCN(c3ccc4c(C5CCC(=O)NC5=O)nn(C)c4c3)CC2)[C@@H](F)C1 ; *COC(=O)N1CC[C@@H](CC2CCN(c3ccc4c(C5CCC(=O)NC5=O)nn(C)c4c3)CC2)[C@H](F)C1 ; *COC(=O)N1CCC(CC2CCN(c3ccc4c(C5CCC(=O)NC5=O)nn(CC)c4c3)CC2)CC1 ; *COC(=O)N1CCC(CC2CCN(c3ccc4c(C5CCC(=O)NC5=O)nn(C(C)C)c4c3)CC2)CC1]

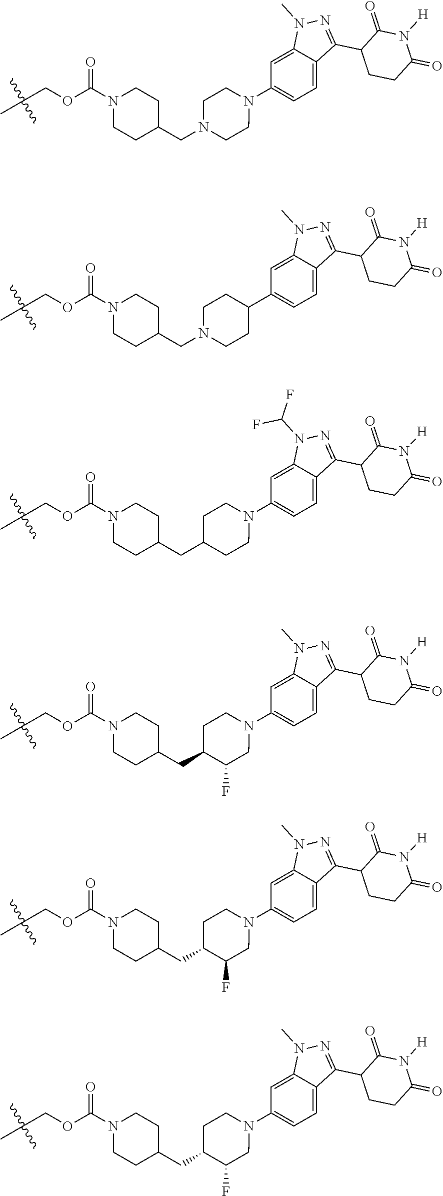 [SMILES: *COC(=O)N1CCC(CN2CCN(c3ccc4c(C5CCC(=O)NC5=O)nn(C)c4c3)CC2)CC1 ; *COC(=O)N1CCC(CN2CCC(c3ccc4c(C5CCC(=O)NC5=O)nn(C)c4c3)CC2)CC1 ; *COC(=O)N1CCC(CC2CCN(c3ccc4c(C5CCC(=O)NC5=O)nn(C(F)F)c4c3)CC2)CC1 ; *COC(=O)N1CCC(C[C@H]2CCN(c3ccc4c(C5CCC(=O)NC5=O)nn(C)c4c3)C[C@@H]2F)CC1 ; *COC(=O)N1CCC(C[C@@H]2CCN(c3ccc4c(C5CCC(=O)NC5=O)nn(C)c4c3)C[C@H]2F)CC1 ; *COC(=O)N1CCC(C[C@@H]2CCN(c3ccc4c(C5CCC(=O)NC5=O)nn(C)c4c3)C[C@@H]2F)CC1]

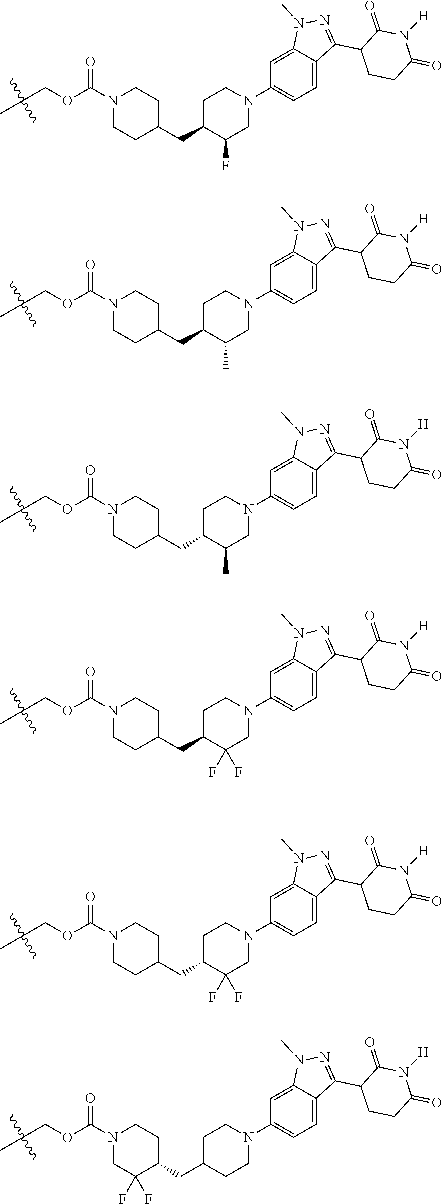 [SMILES: *COC(=O)N1CCC(C[C@H]2CCN(c3ccc4c(C5CCC(=O)NC5=O)nn(C)c4c3)C[C@H]2F)CC1 ; *COC(=O)N1CCC(C[C@H]2CCN(c3ccc4c(C5CCC(=O)NC5=O)nn(C)c4c3)C[C@@H]2C)CC1 ; *COC(=O)N1CCC(C[C@@H]2CCN(c3ccc4c(C5CCC(=O)NC5=O)nn(C)c4c3)C[C@H]2C)CC1 ; *COC(=O)N1CCC(C[C@H]2CCN(c3ccc4c(C5CCC(=O)NC5=O)nn(C)c4c3)CC2(F)F)CC1 ; *COC(=O)N1CCC(C[C@@H]2CCN(c3ccc4c(C5CCC(=O)NC5=O)nn(C)c4c3)CC2(F)F)CC1 ; *COC(=O)N1CC[C@H](CC2CCN(c3ccc4c(C5CCC(=O)NC5=O)nn(C)c4c3)CC2)C(F)(F)C1]

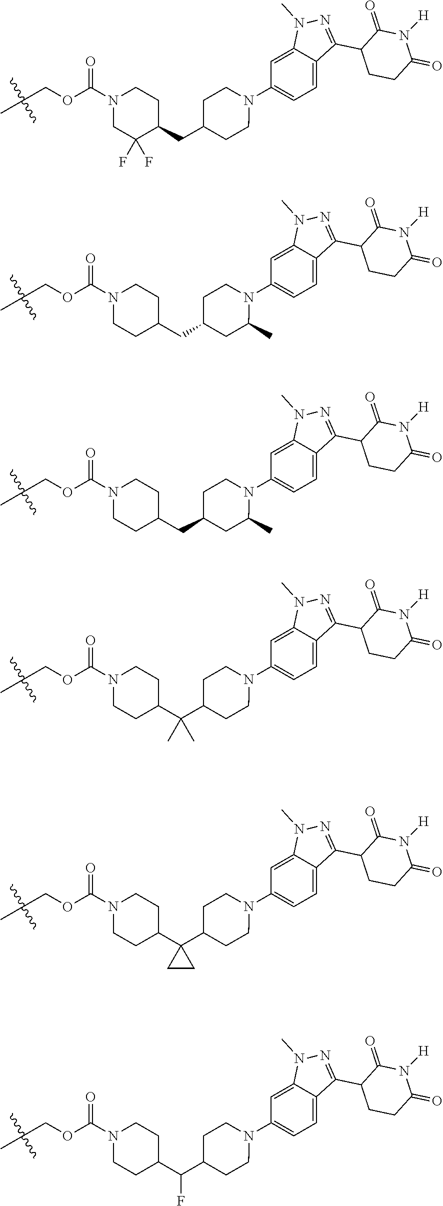 [SMILES: *COC(=O)N1CC[C@@H](CC2CCN(c3ccc4c(C5CCC(=O)NC5=O)nn(C)c4c3)CC2)C(F)(F)C1 ; *COC(=O)N1CCC(C[C@@H]2CCN(c3ccc4c(C5CCC(=O)NC5=O)nn(C)c4c3)[C@@H](C)C2)CC1 ; *COC(=O)N1CCC(C[C@H]2CCN(c3ccc4c(C5CCC(=O)NC5=O)nn(C)c4c3)[C@@H](C)C2)CC1 ; *COC(=O)N1CCC(C(C)(C)C2CCN(c3ccc4c(C5CCC(=O)NC5=O)nn(C)c4c3)CC2)CC1 ; *COC(=O)N1CCC(C2(C3CCN(c4ccc5c(C6CCC(=O)NC6=O)nn(C)c5c4)CC3)CC2)CC1 ; *COC(=O)N1CCC(C(F)C2CCN(c3ccc4c(C5CCC(=O)NC5=O)nn(C)c4c3)CC2)CC1]

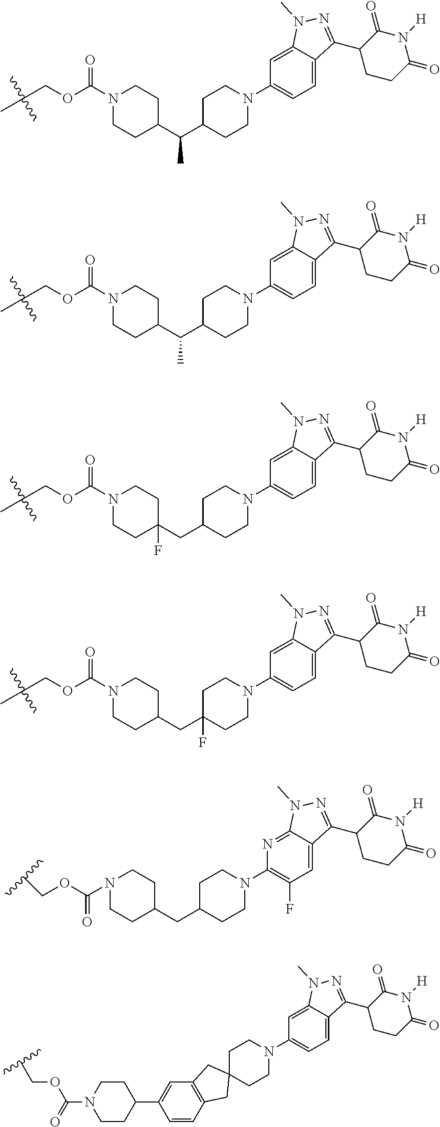 [SMILES: *COC(=O)N1CCC([C@H](C)C2CCN(c3ccc4c(C5CCC(=O)NC5=O)nn(C)c4c3)CC2)CC1 ; *COC(=O)N1CCC([C@@H](C)C2CCN(c3ccc4c(C5CCC(=O)NC5=O)nn(C)c4c3)CC2)CC1 ; *COC(=O)N1CCC(F)(CC2CCN(c3ccc4c(C5CCC(=O)NC5=O)nn(C)c4c3)CC2)CC1 ; *COC(=O)N1CCC(CC2(F)CCN(c3ccc4c(C5CCC(=O)NC5=O)nn(C)c4c3)CC2)CC1 ; *COC(=O)N1CCC(CC2CCN(c3nc4c(cc3F)c(C3CCC(=O)NC3=O)nn4C)CC2)CC1 ; *COC(=O)N1CCC(c2ccc3c(c2)CC2(CCN(c4ccc5c(C6CCC(=O)NC6=O)nn(C)c5c4)CC2)C3)CC1]

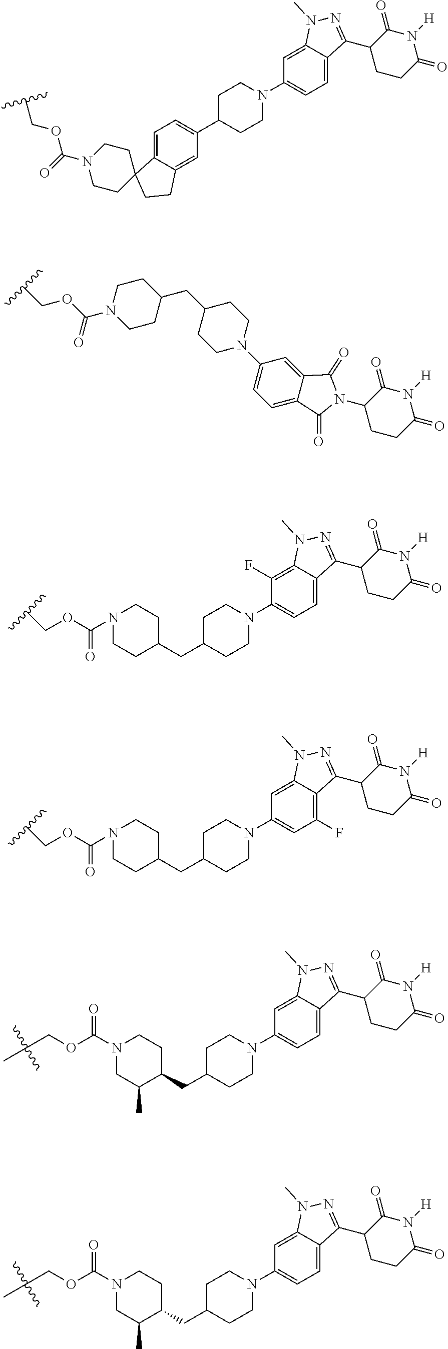 [SMILES: *COC(=O)N1CCC2(CCc3cc(C4CCN(c5ccc6c(C7CCC(=O)NC7=O)nn(C)c6c5)CC4)ccc32)CC1 ; *COC(=O)N1CCC(CC2CCN(c3ccc4c(c3)C(=O)N(C3CCC(=O)NC3=O)C4=O)CC2)CC1 ; *COC(=O)N1CCC(CC2CCN(c3ccc4c(C5CCC(=O)NC5=O)nn(C)c4c3F)CC2)CC1 ; *COC(=O)N1CCC(CC2CCN(c3cc(F)c4c(C5CCC(=O)NC5=O)nn(C)c4c3)CC2)CC1 ; *COC(=O)N1CC[C@@H](CC2CCN(c3ccc4c(C5CCC(=O)NC5=O)nn(C)c4c3)CC2)[C@@H](C)C1 ; *COC(=O)N1CC[C@H](CC2CCN(c3ccc4c(C5CCC(=O)NC5=O)nn(C)c4c3)CC2)[C@@H](C)C1]

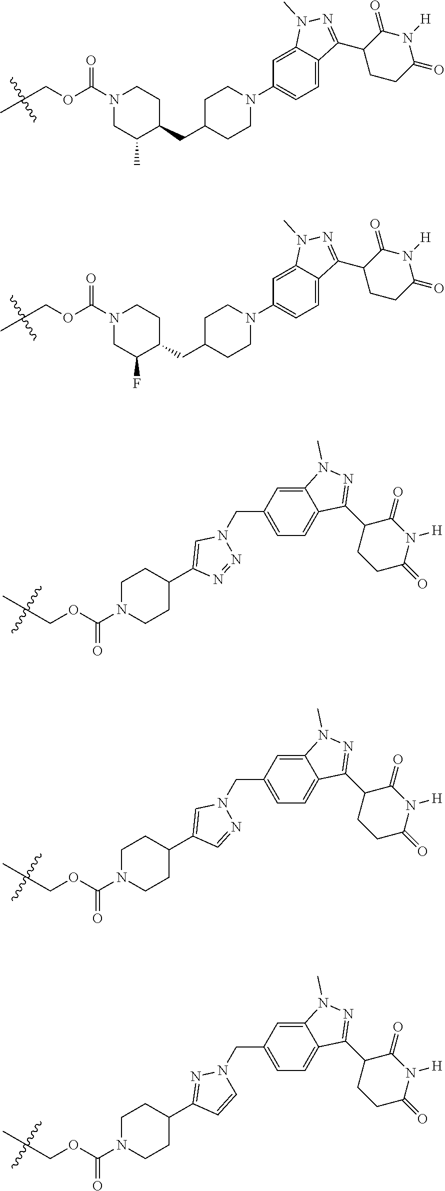 [SMILES: *COC(=O)N1CC[C@@H](CC2CCN(c3ccc4c(C5CCC(=O)NC5=O)nn(C)c4c3)CC2)[C@H](C)C1 ; *COC(=O)N1CC[C@H](CC2CCN(c3ccc4c(C5CCC(=O)NC5=O)nn(C)c4c3)CC2)[C@@H](F)C1 ; *COC(=O)N1CCC(c2cn(Cc3ccc4c(C5CCC(=O)NC5=O)nn(C)c4c3)nn2)CC1 ; *COC(=O)N1CCC(c2cnn(Cc3ccc4c(C5CCC(=O)NC5=O)nn(C)c4c3)c2)CC1 ; *COC(=O)N1CCC(c2ccn(Cc3ccc4c(C5CCC(=O)NC5=O)nn(C)c4c3)n2)CC1]

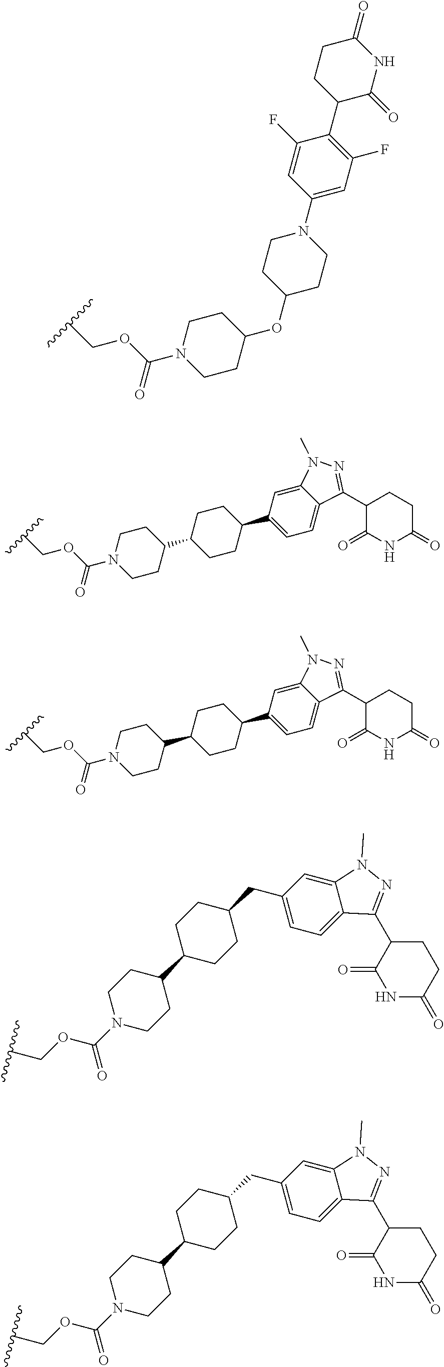 [SMILES: *COC(=O)N1CCC(OC2CCN(c3cc(F)c(C4CCC(=O)NC4=O)c(F)c3)CC2)CC1 ; *COC(=O)N1CCC([C@H]2CC[C@H](c3ccc4c(C5CCC(=O)NC5=O)nn(C)c4c3)CC2)CC1 ; *COC(=O)N1CCC([C@H]2CC[C@@H](c3ccc4c(C5CCC(=O)NC5=O)nn(C)c4c3)CC2)CC1 ; *COC(=O)N1CCC([C@H]2CC[C@@H](Cc3ccc4c(C5CCC(=O)NC5=O)nn(C)c4c3)CC2)CC1 ; *COC(=O)N1CCC([C@H]2CC[C@H](Cc3ccc4c(C5CCC(=O)NC5=O)nn(C)c4c3)CC2)CC1]

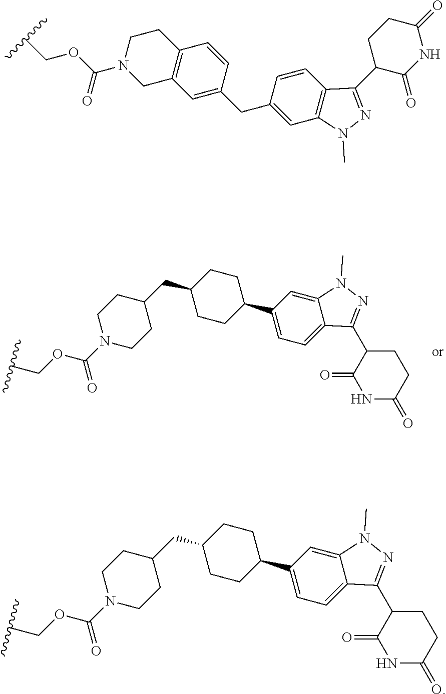 [SMILES: *COC(=O)N1CCc2ccc(Cc3ccc4c(C5CCC(=O)NC5=O)nn(C)c4c3)cc2C1 ; *COC(=O)N1CCC(C[C@H]2CC[C@@H](c3ccc4c(C5CCC(=O)NC5=O)nn(C)c4c3)CC2)CC1 ; *COC(=O)N1CCC(C[C@H]2CC[C@H](c3ccc4c(C5CCC(=O)NC5=O)nn(C)c4c3)CC2)CC1]

51. The compound of claim 1, or pharmaceutically acceptable salt thereof, wherein the compound has the structure:

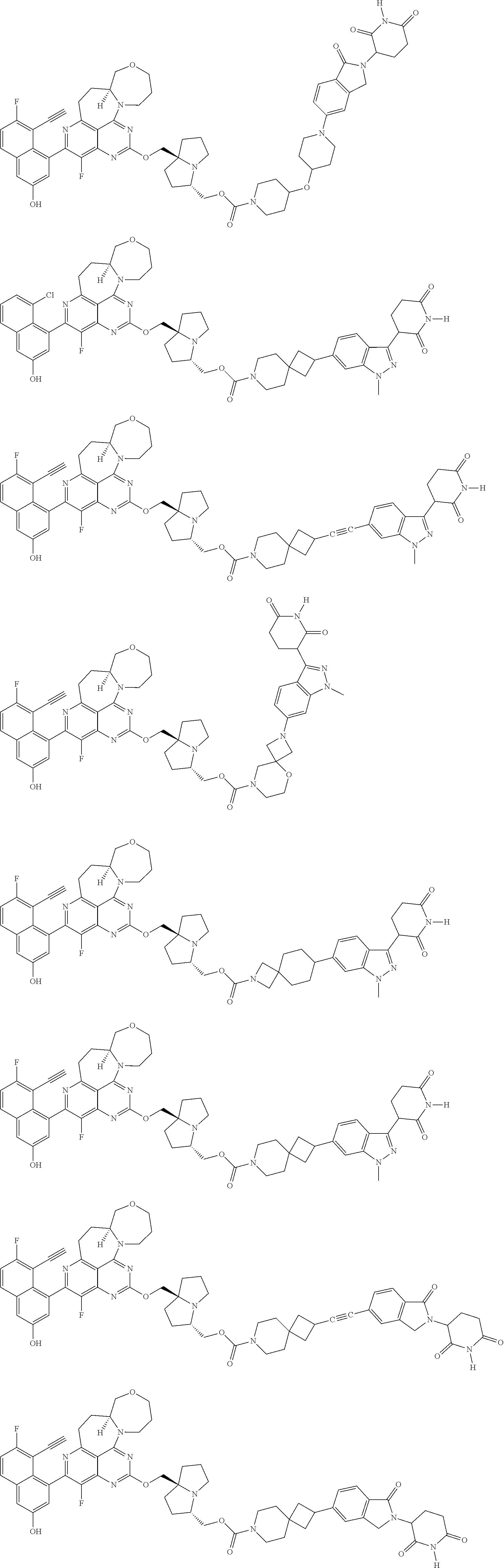

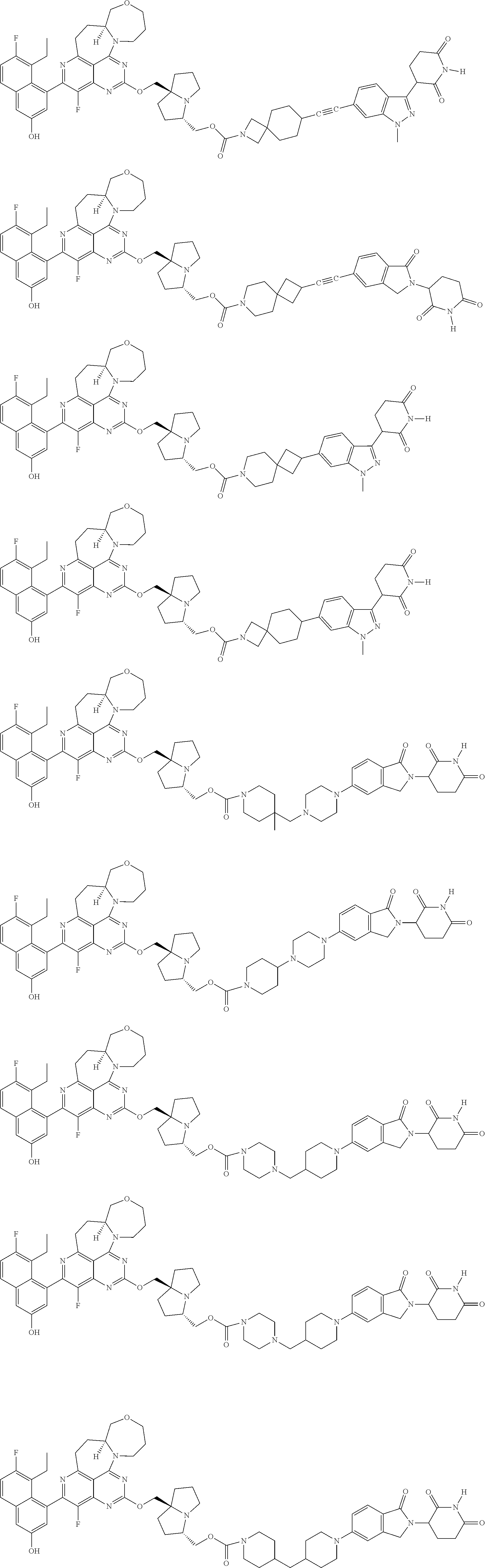

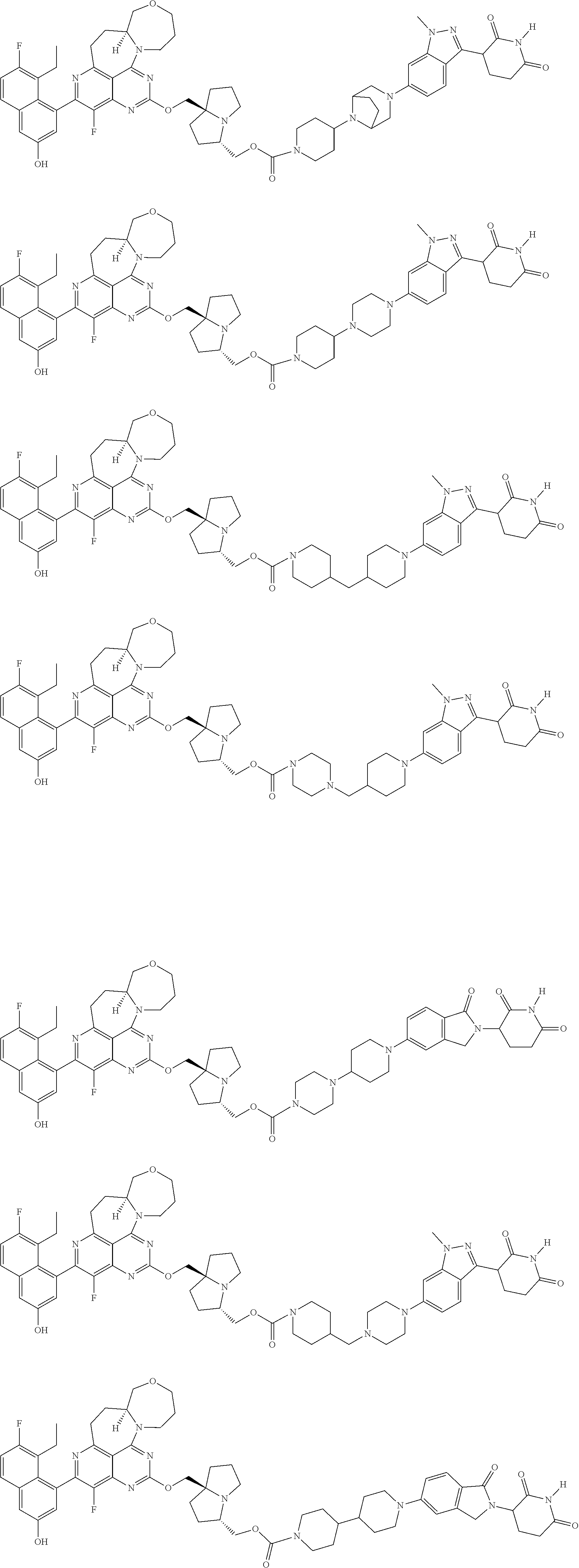

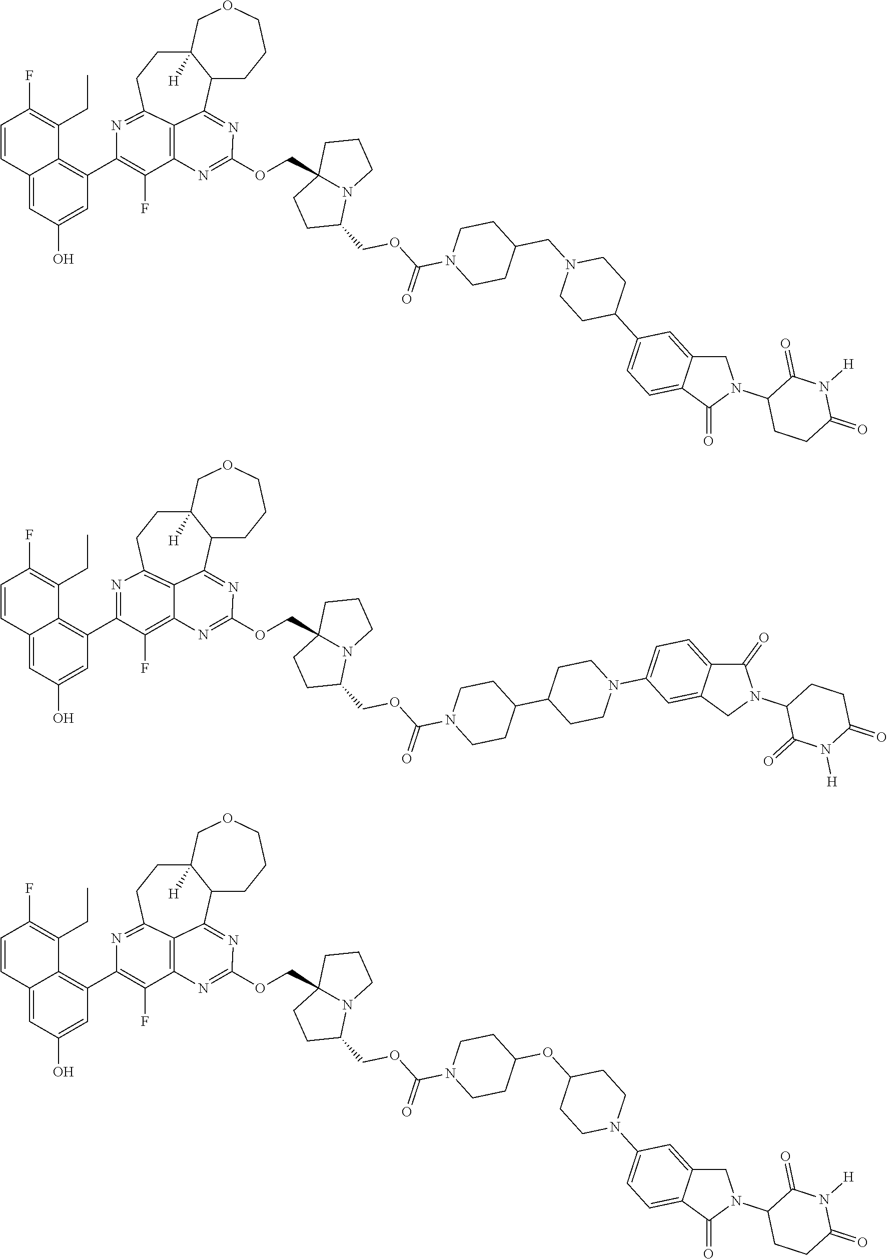

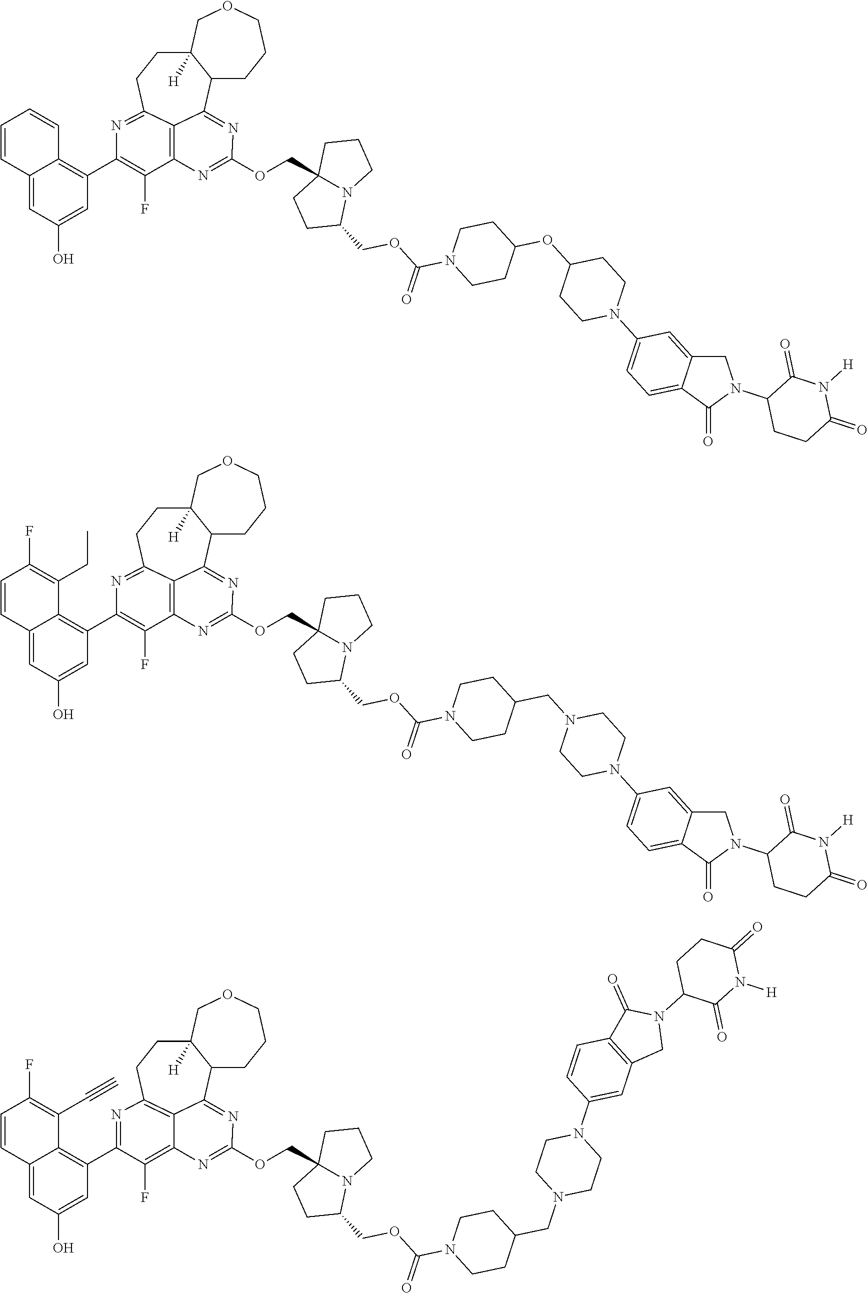

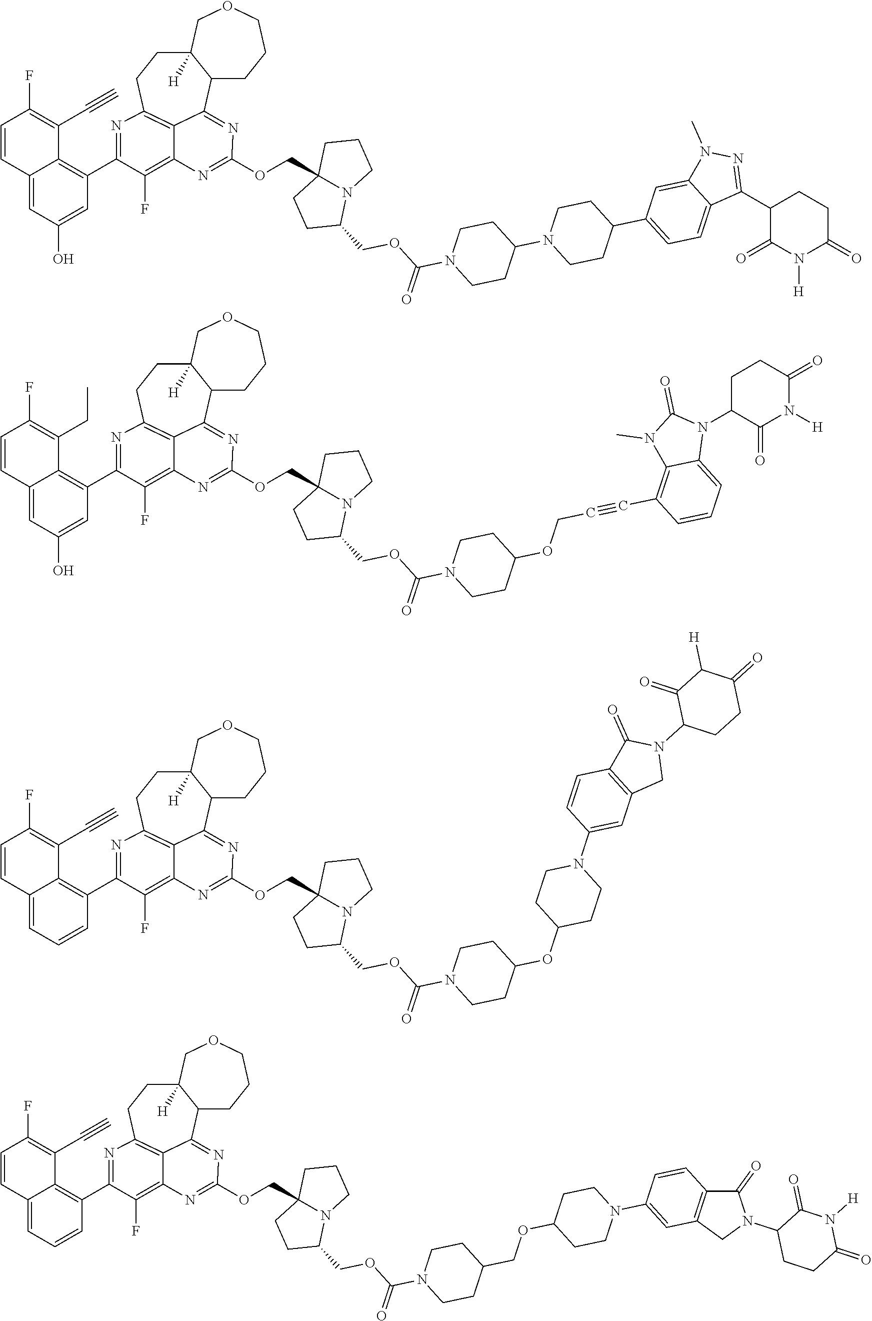

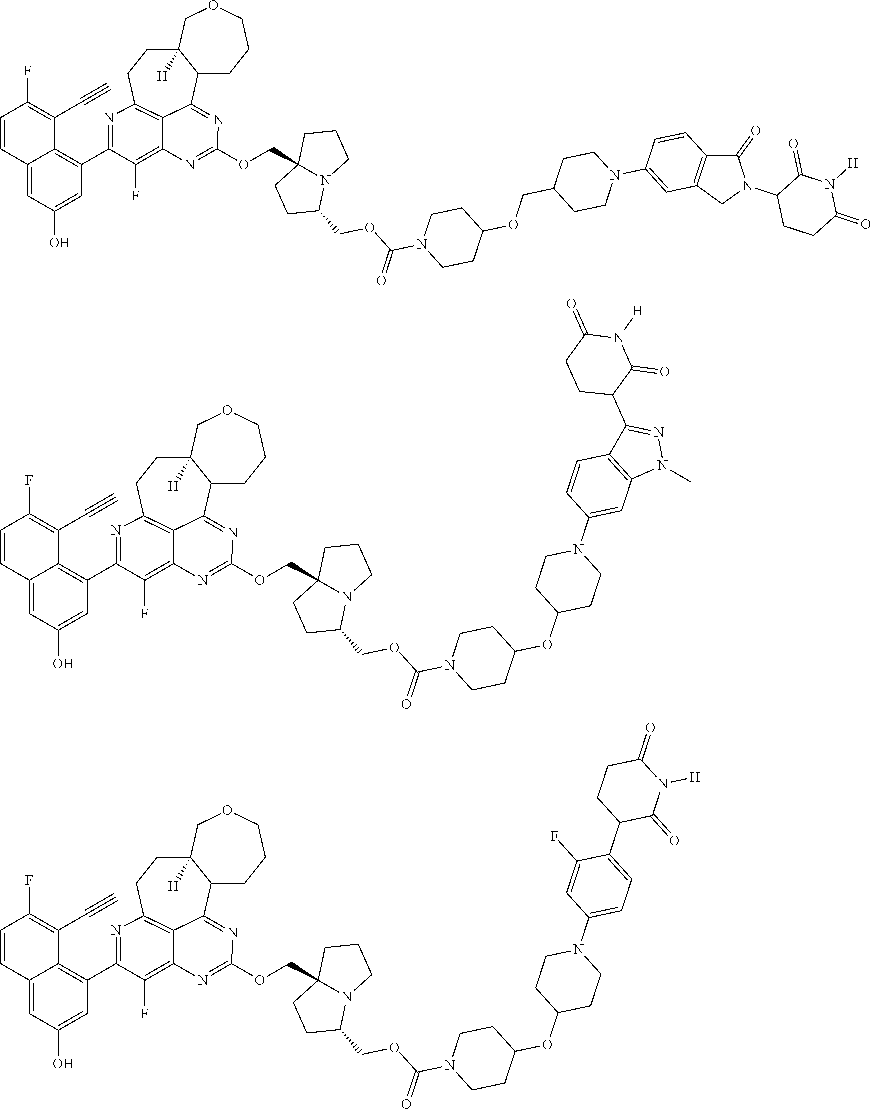

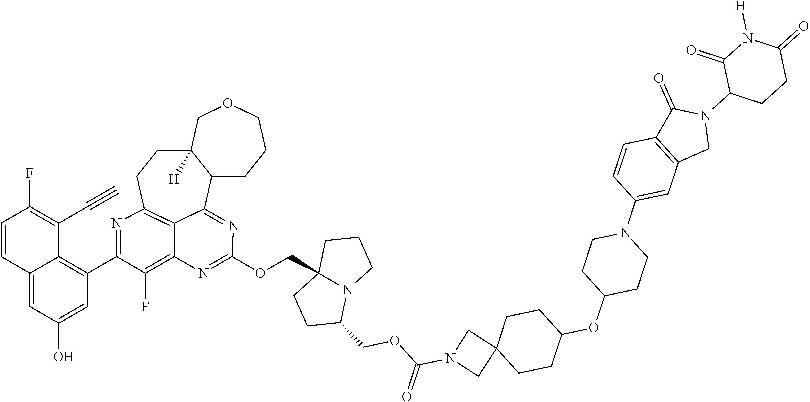

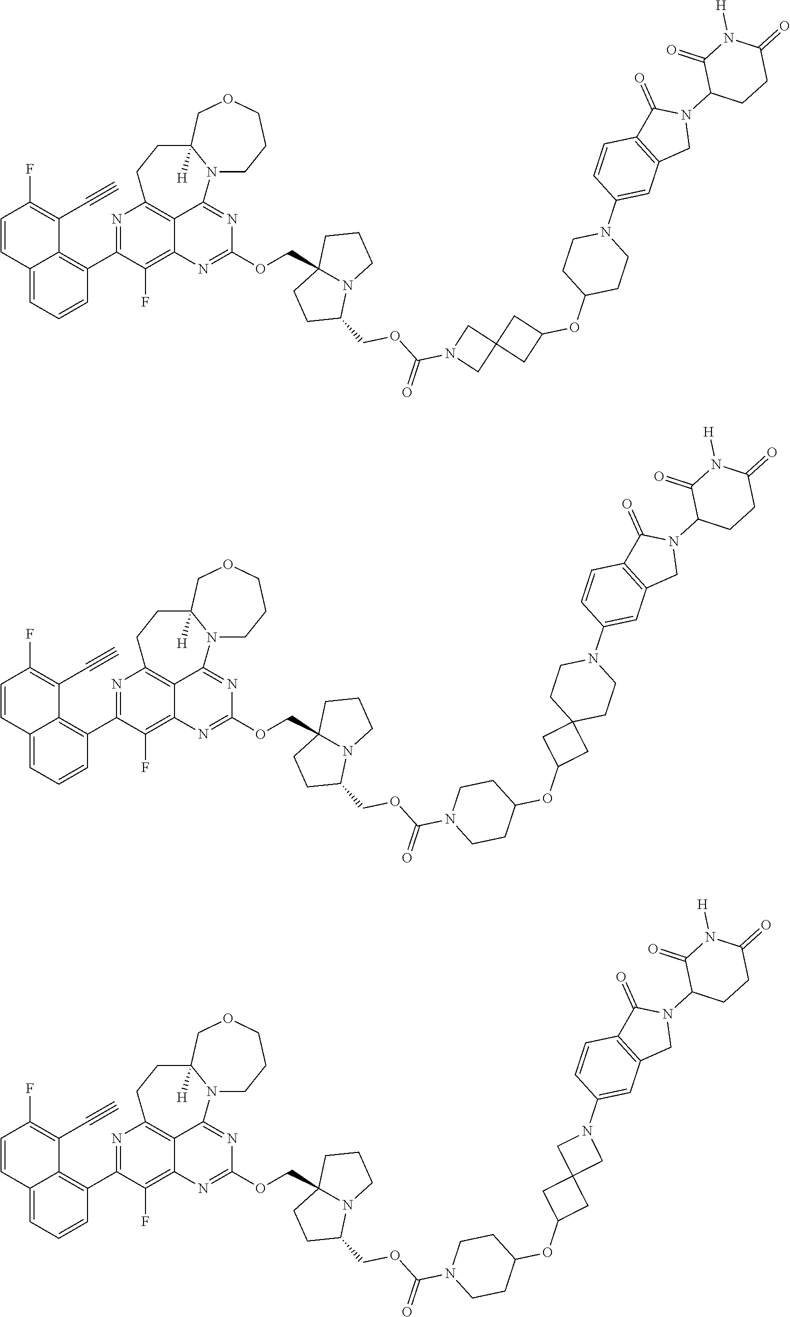

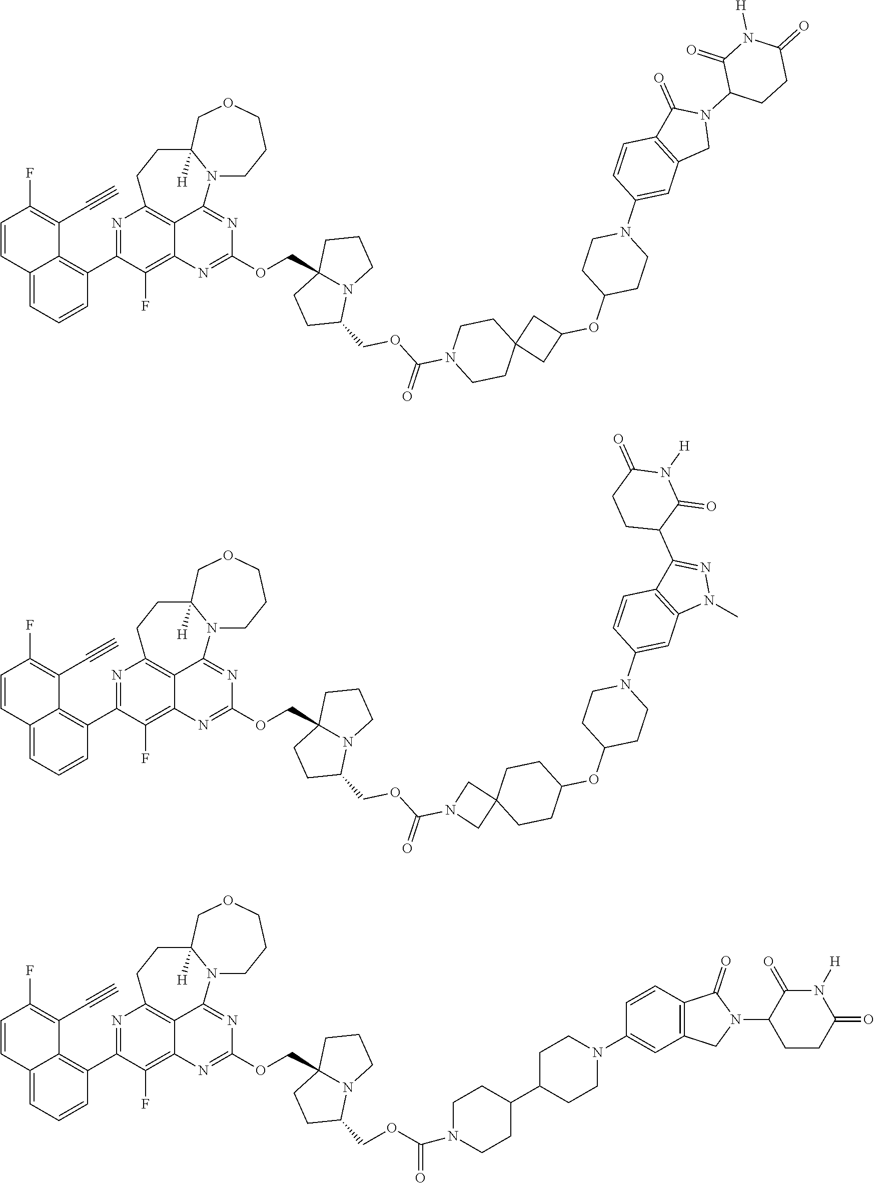

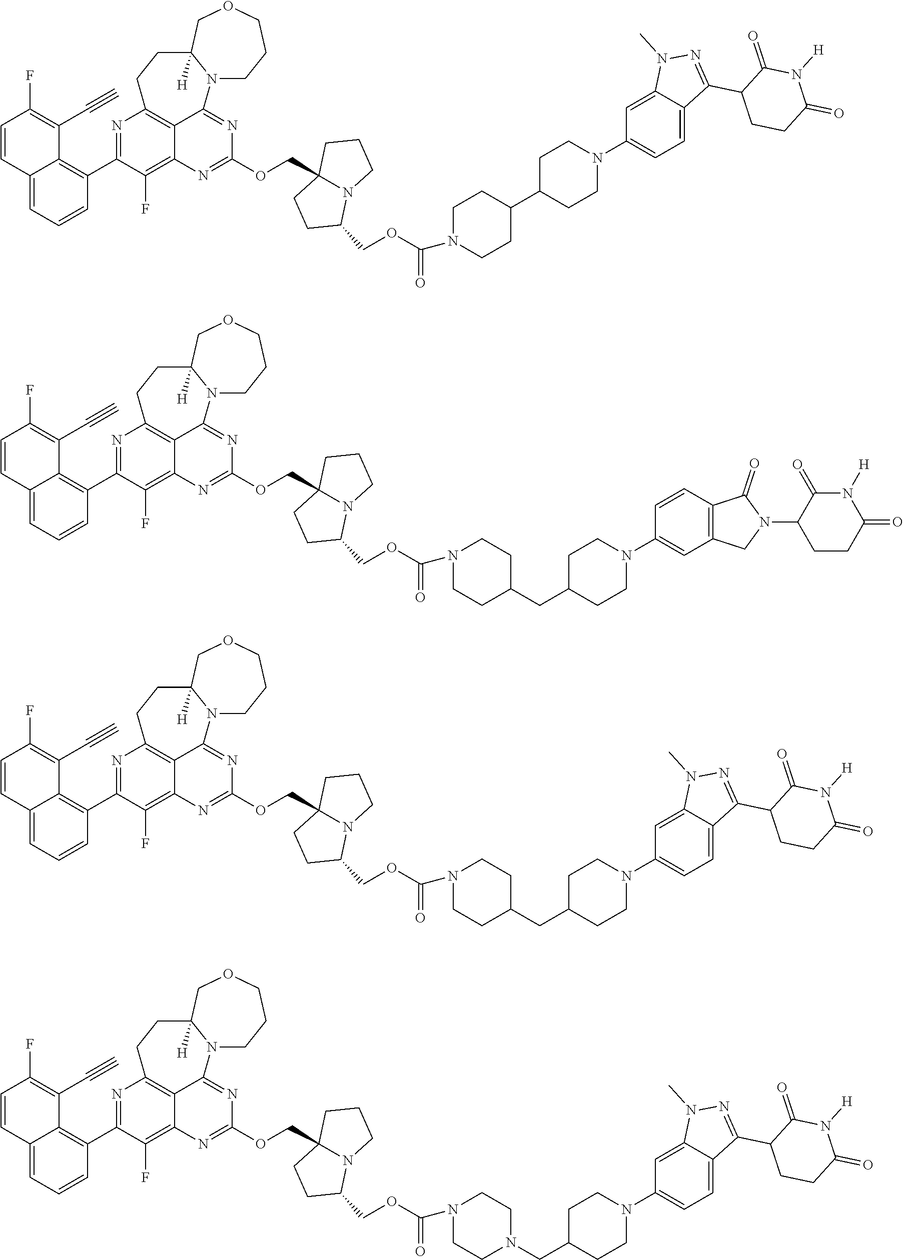

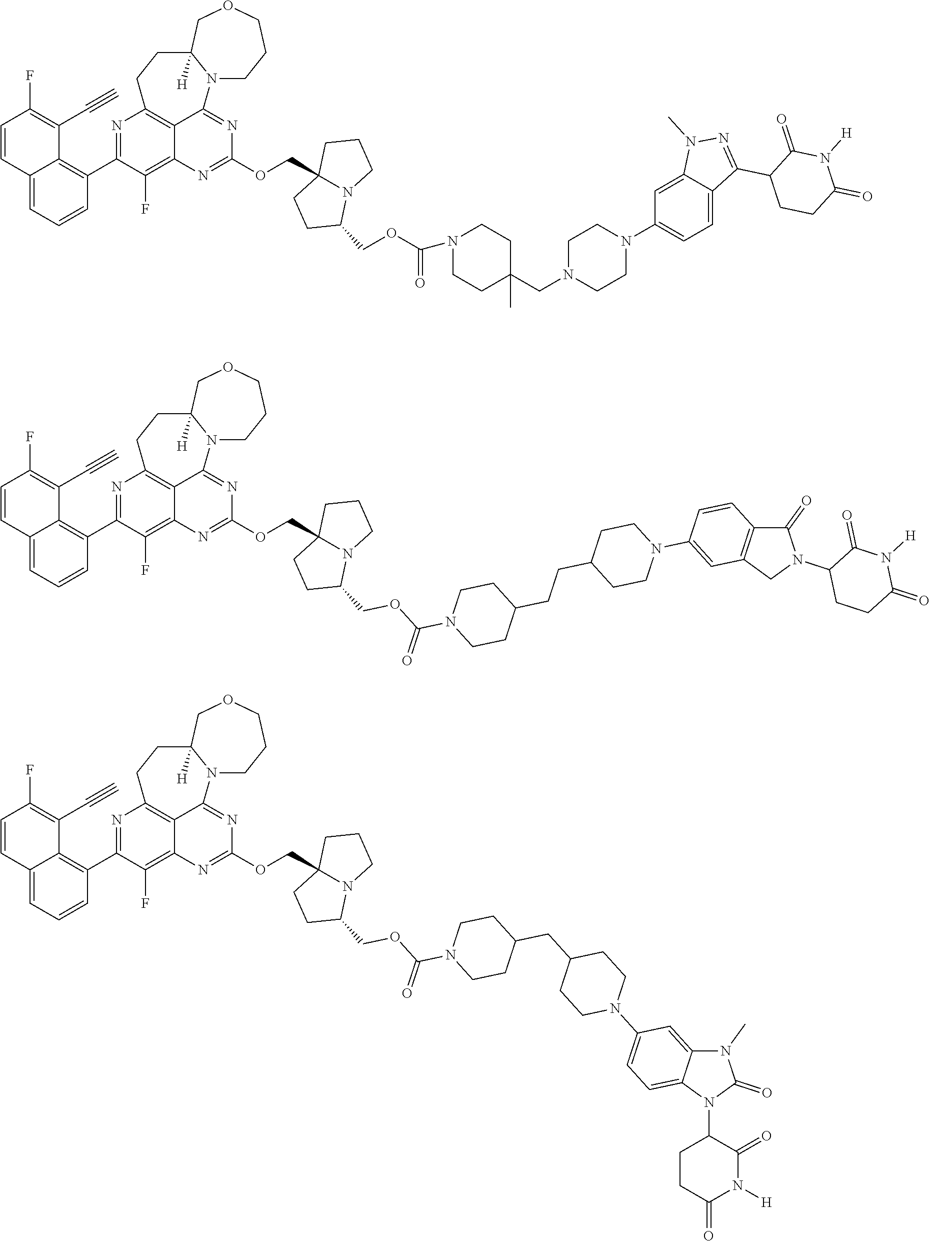

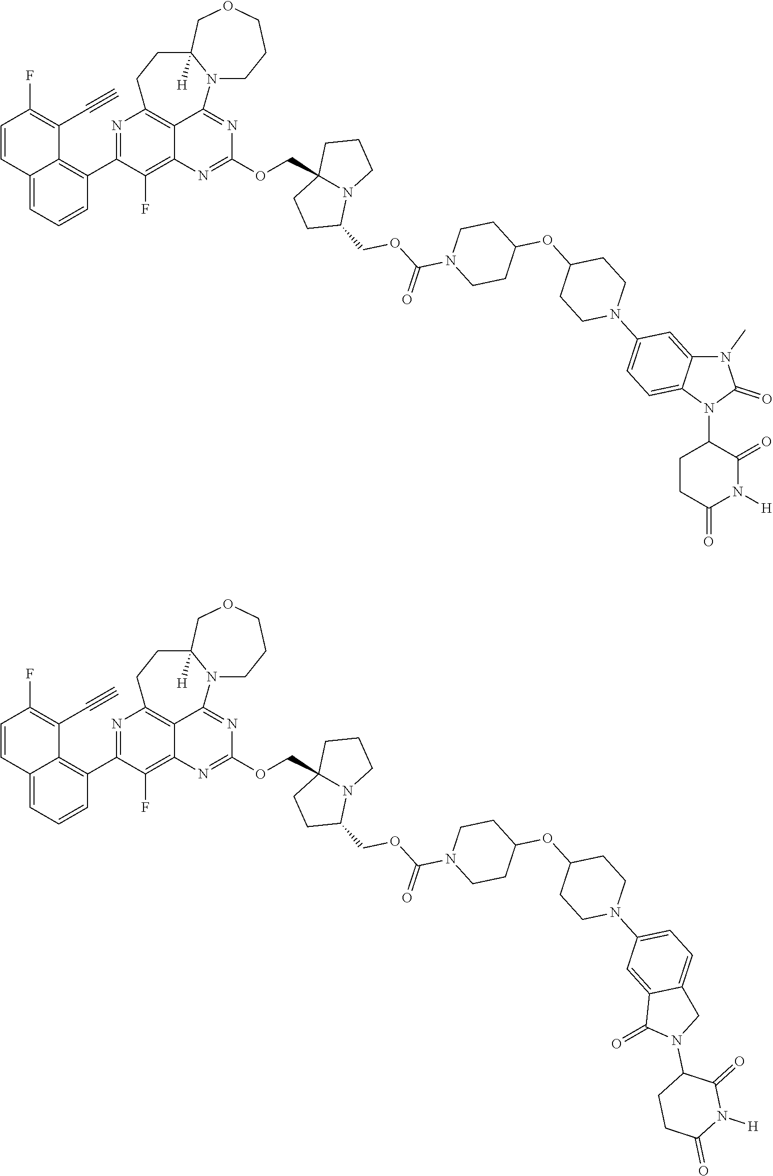

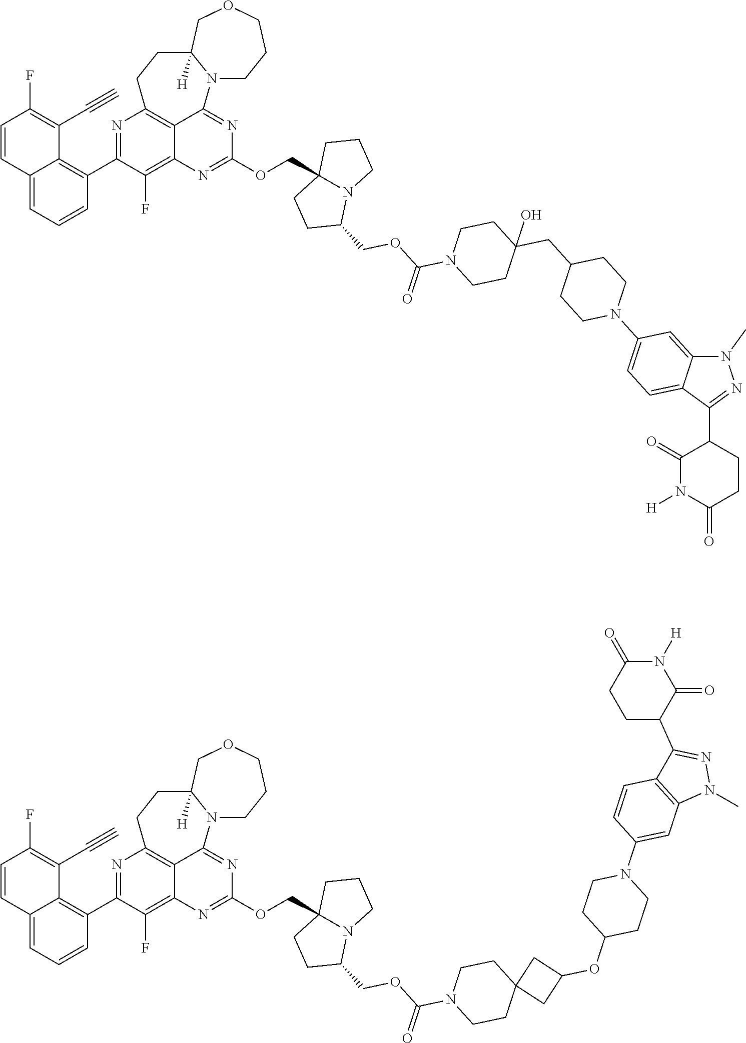

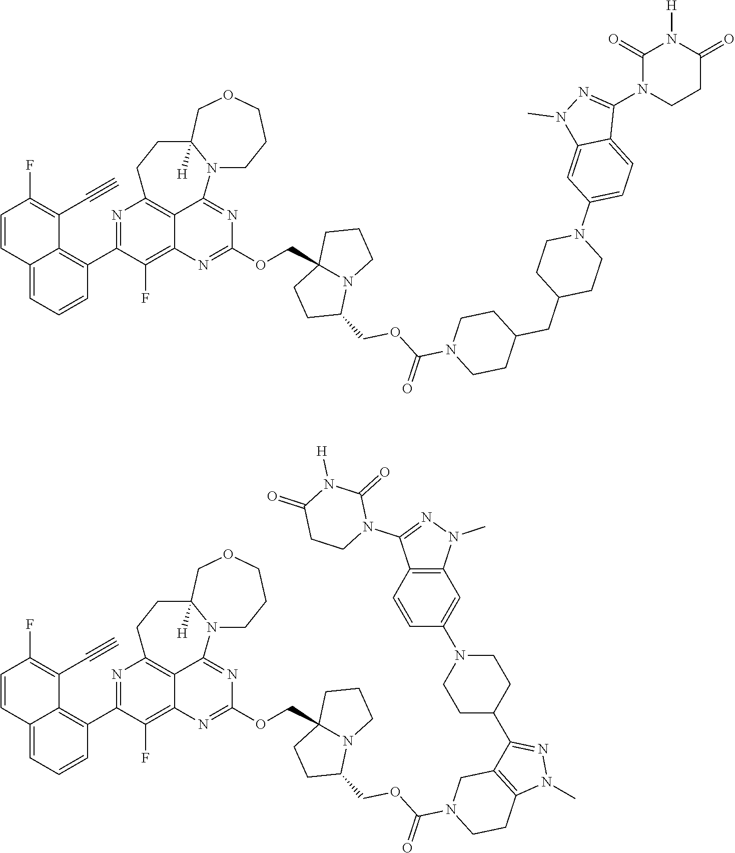

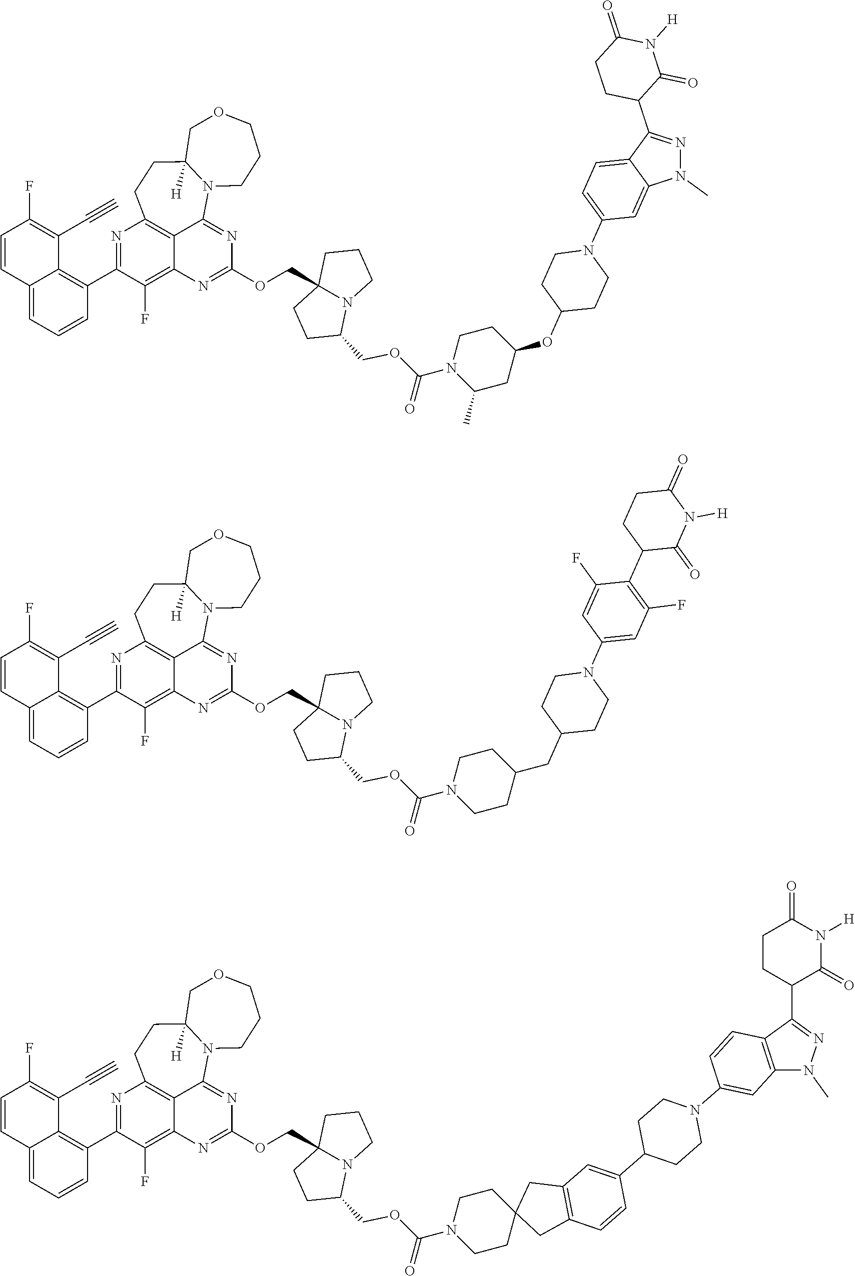

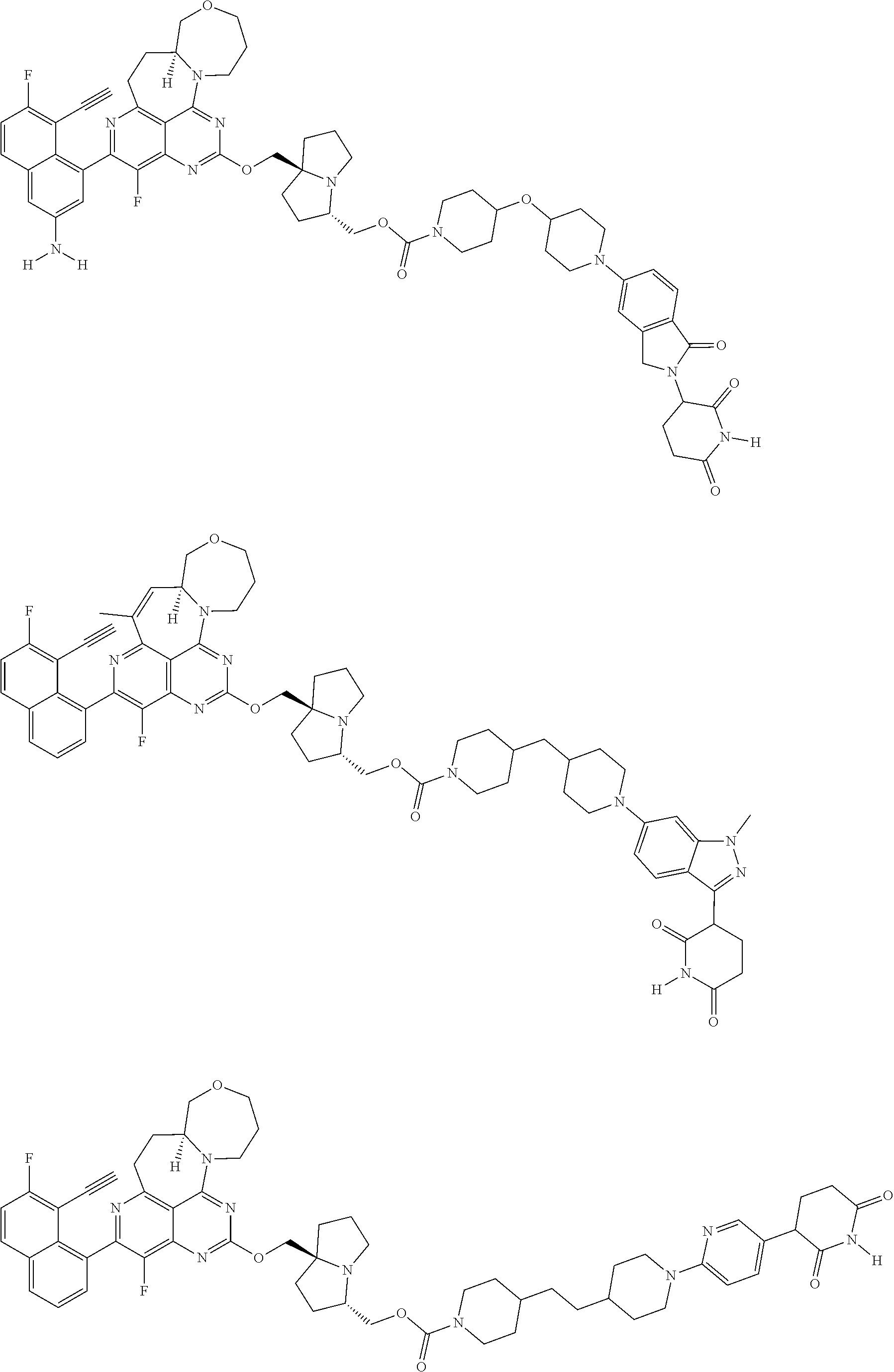

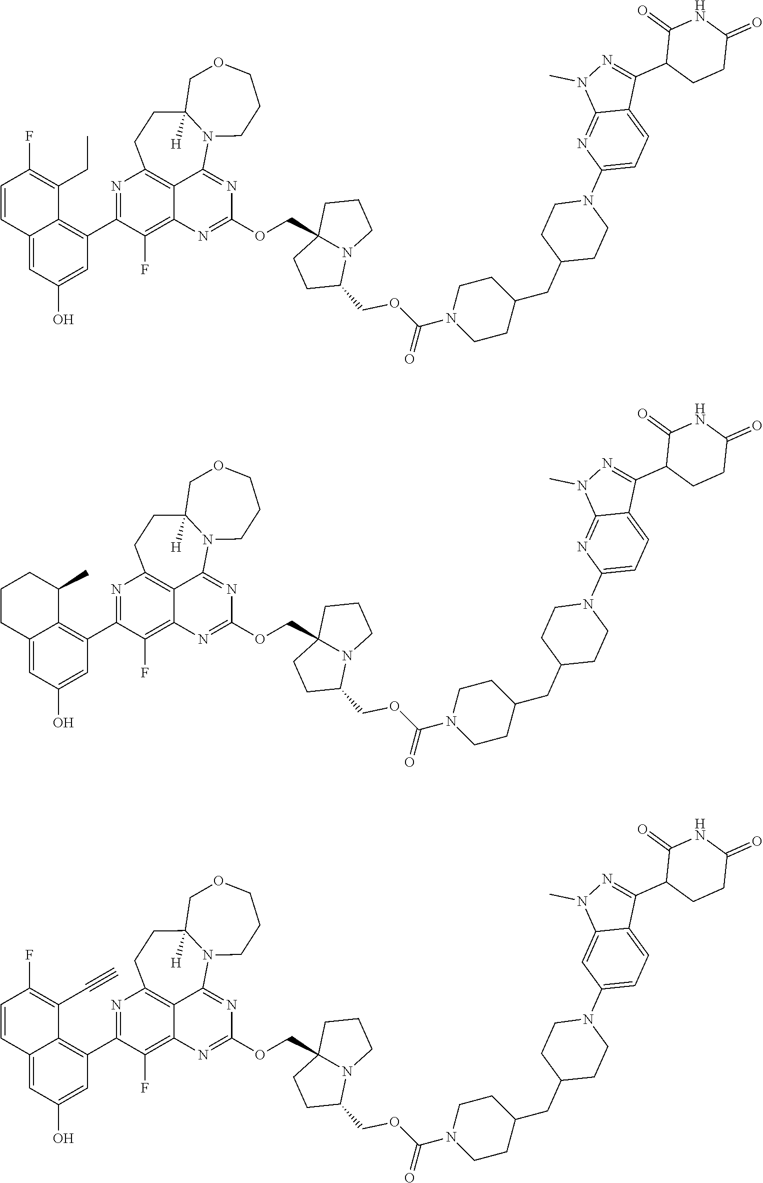

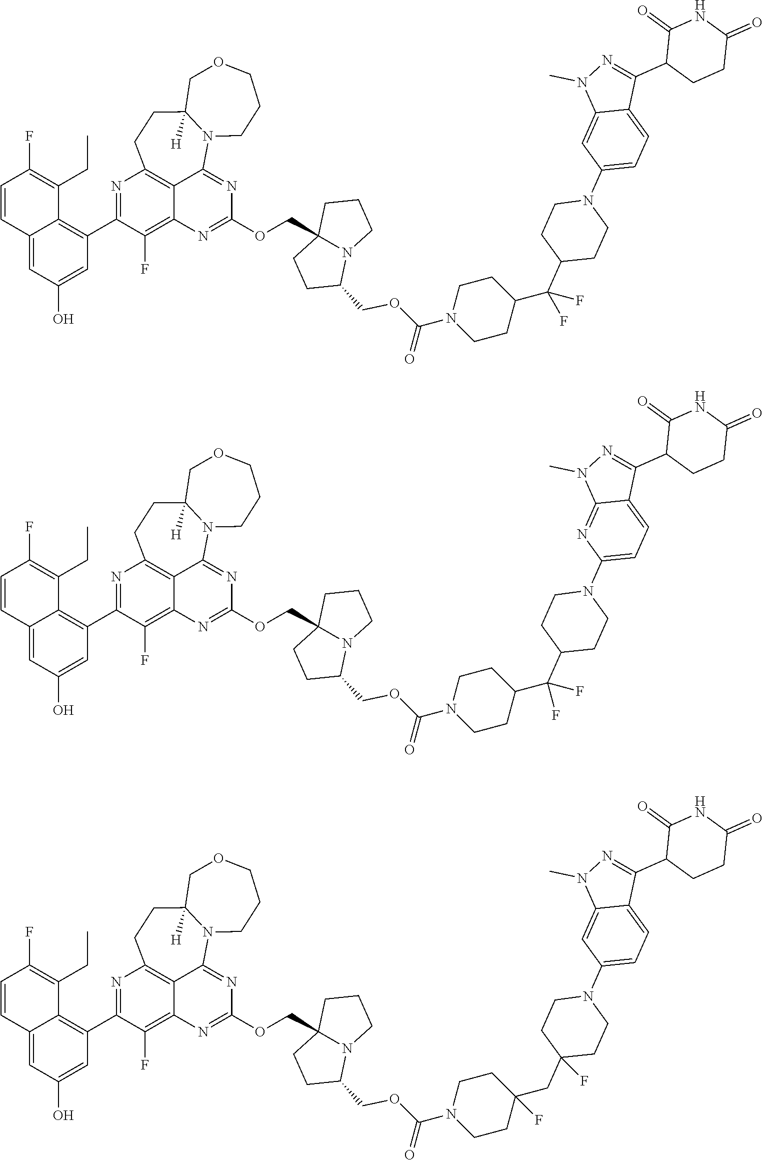

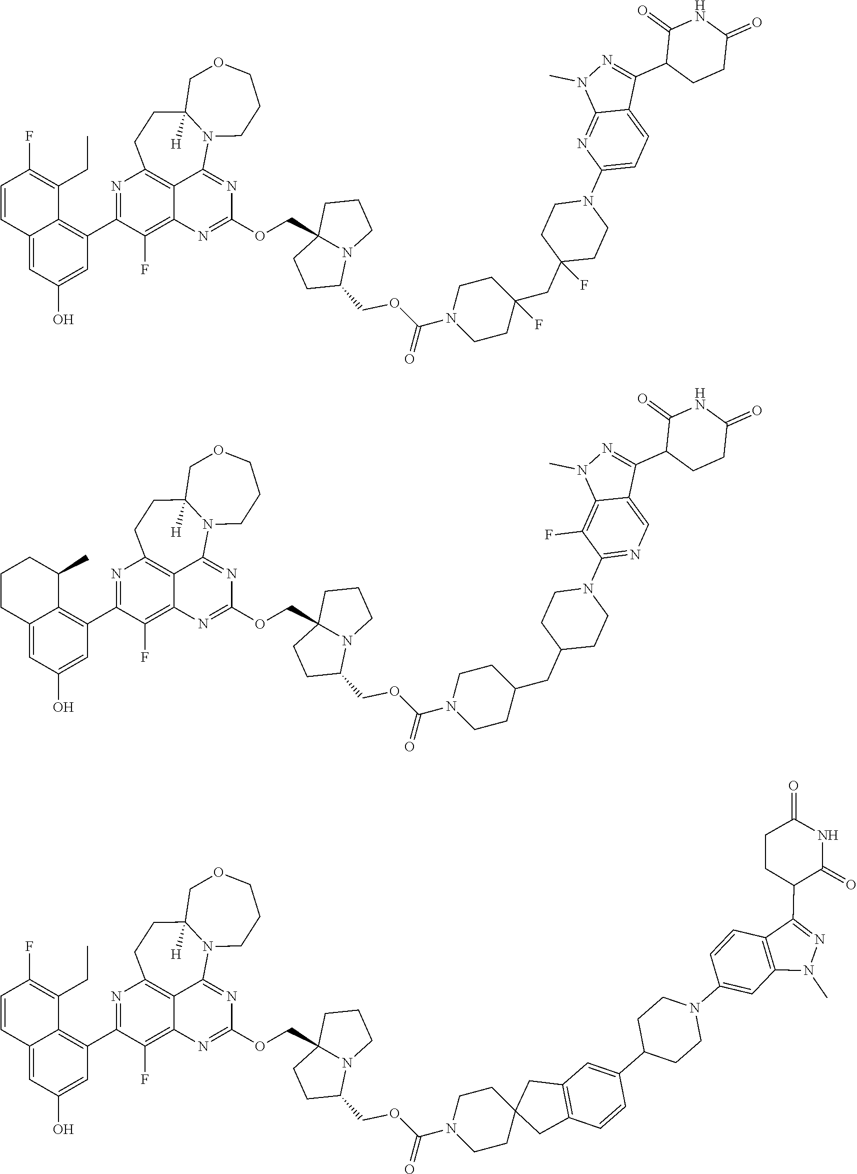

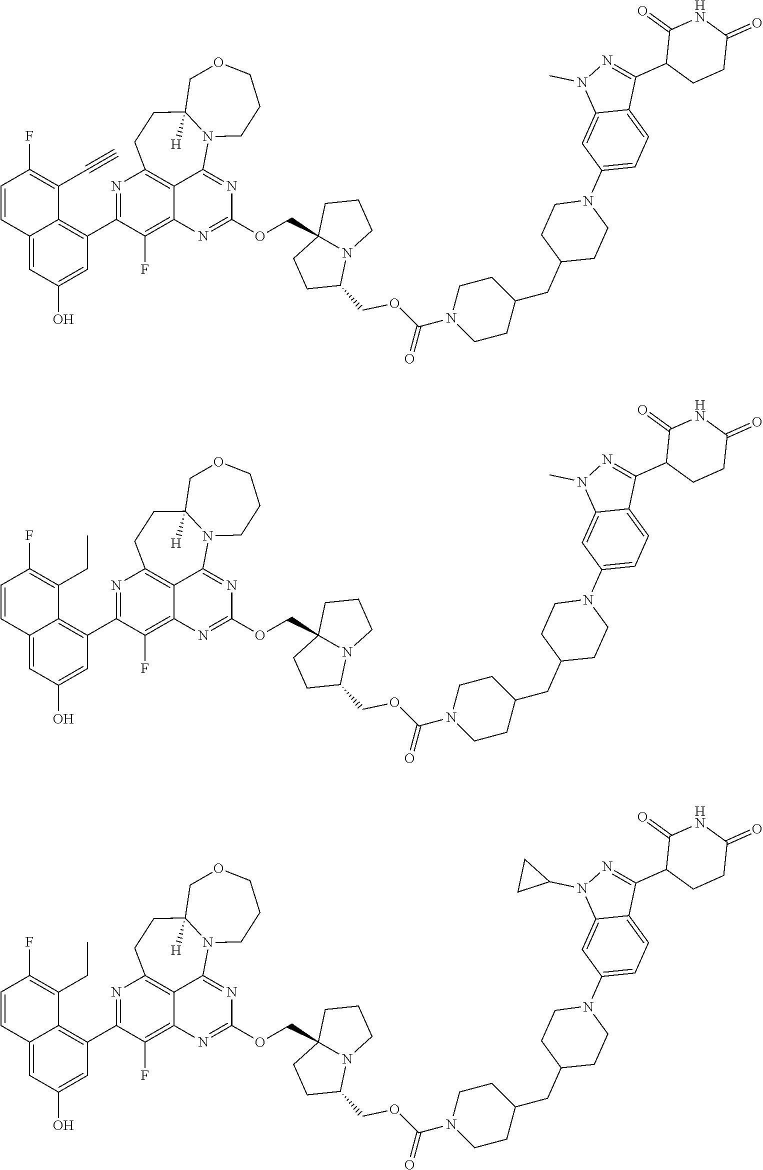

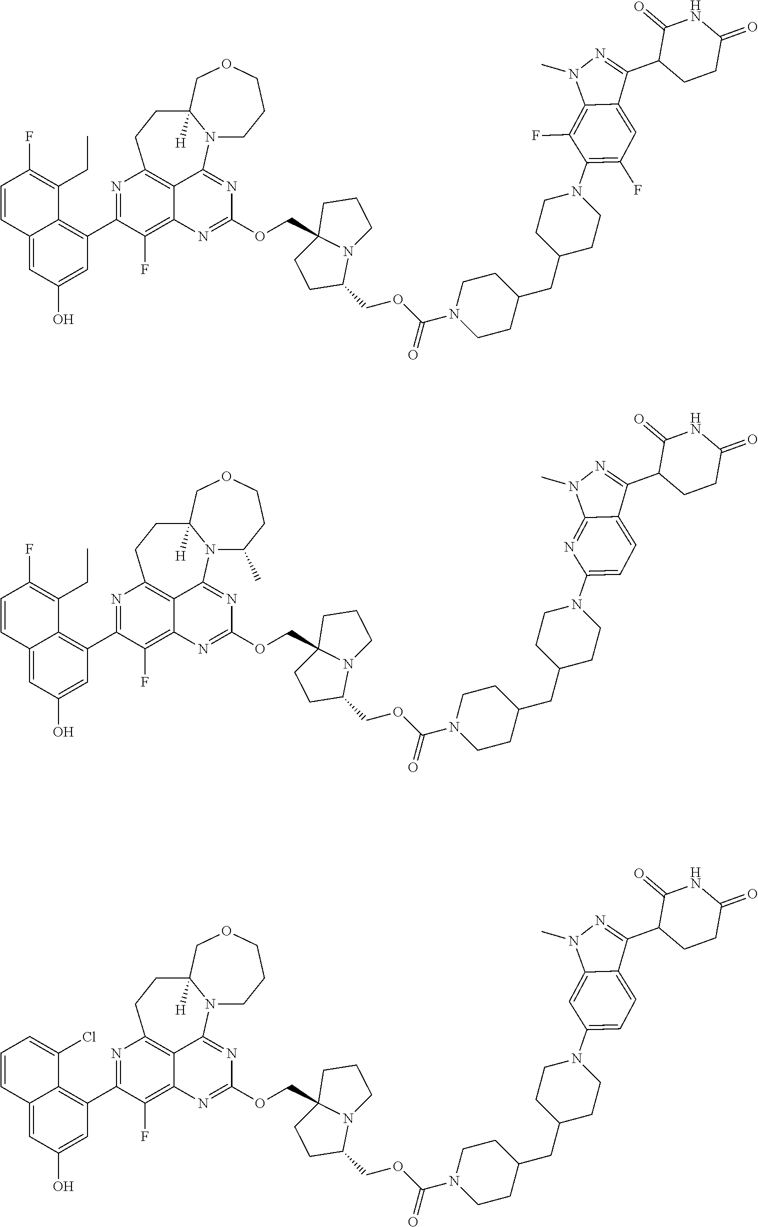

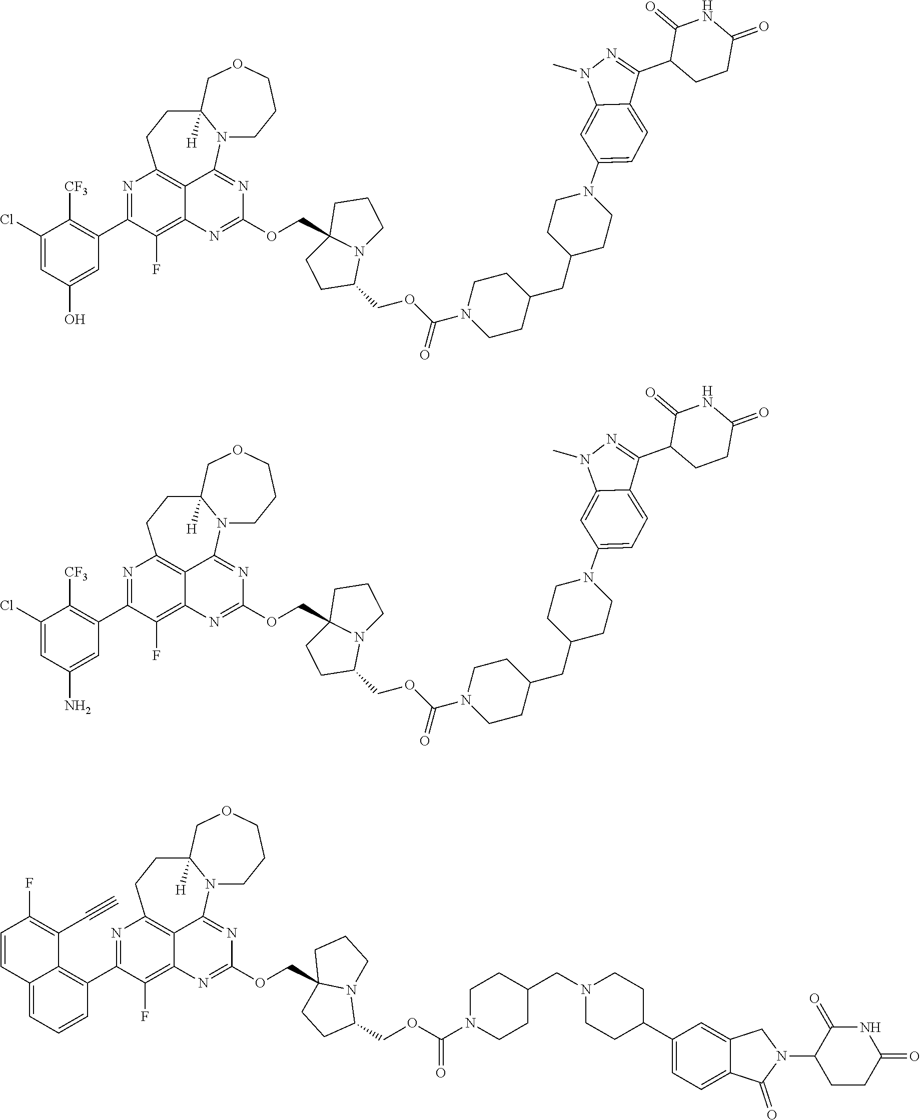

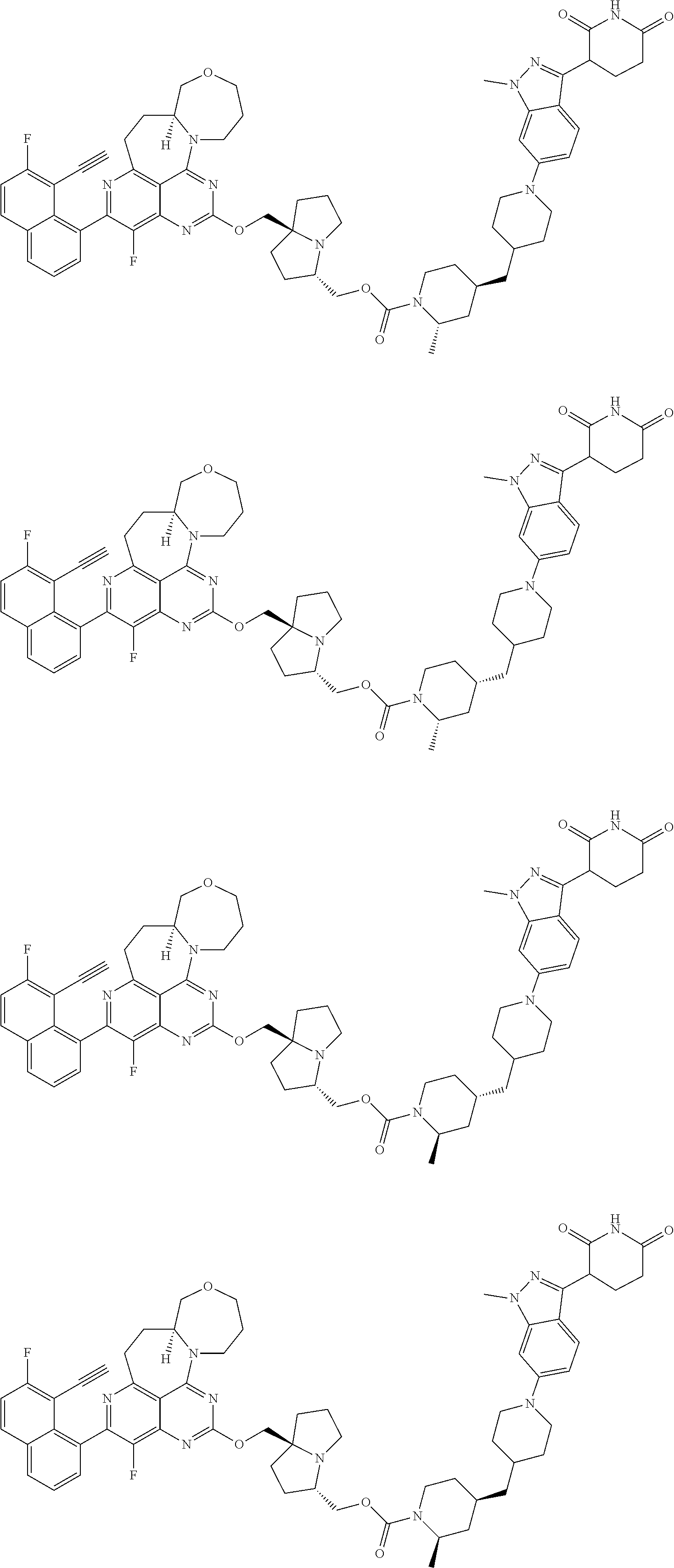

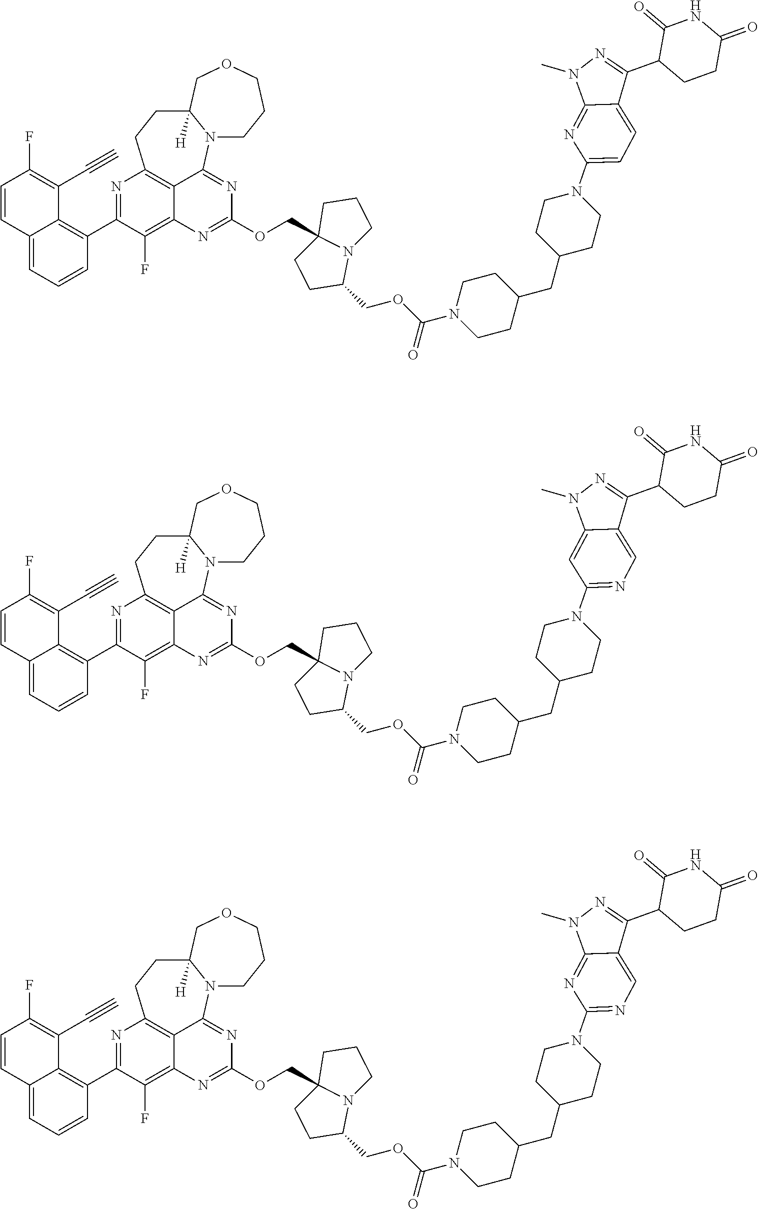

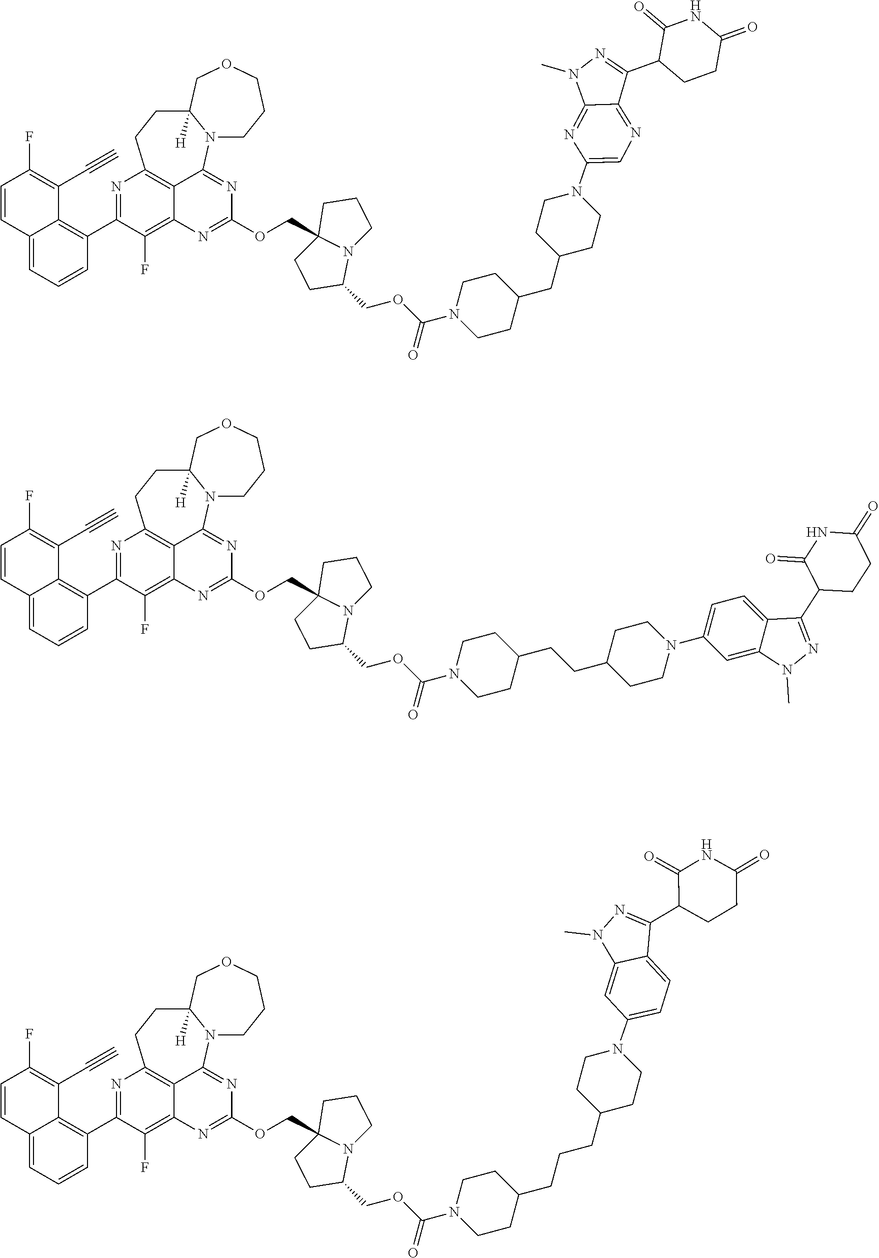

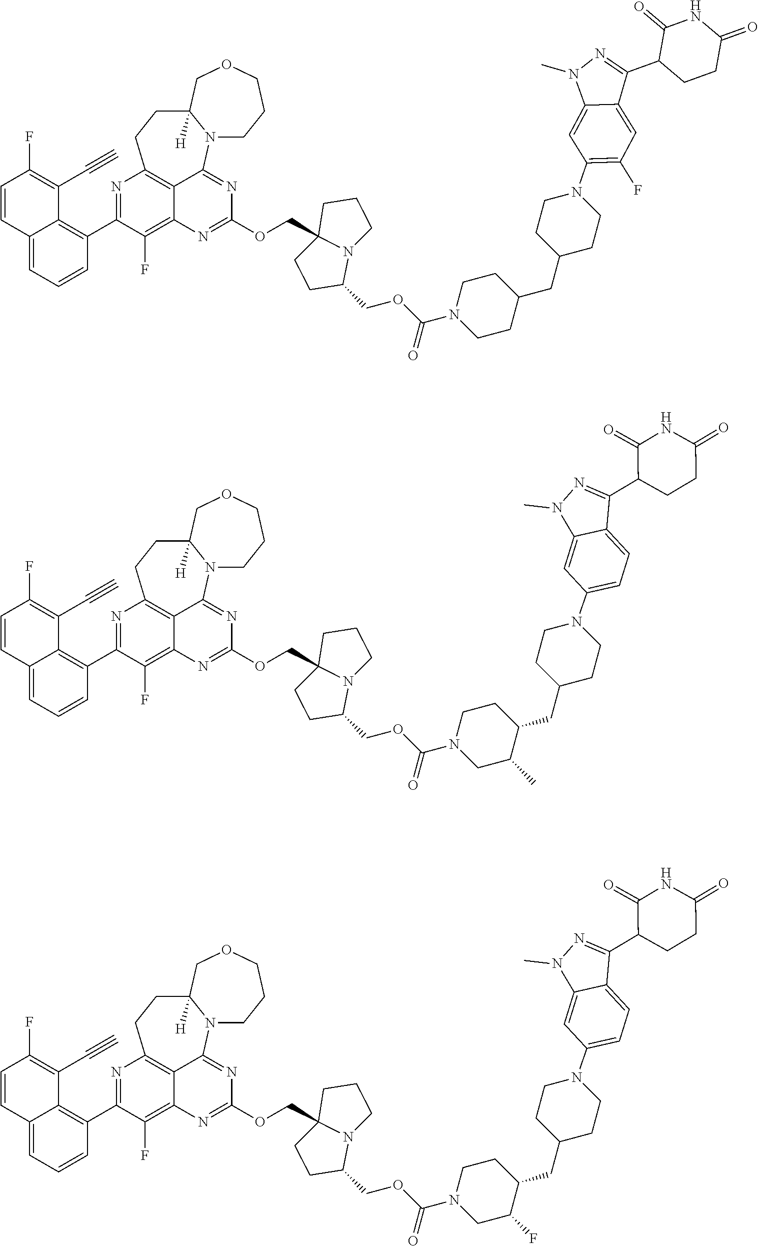

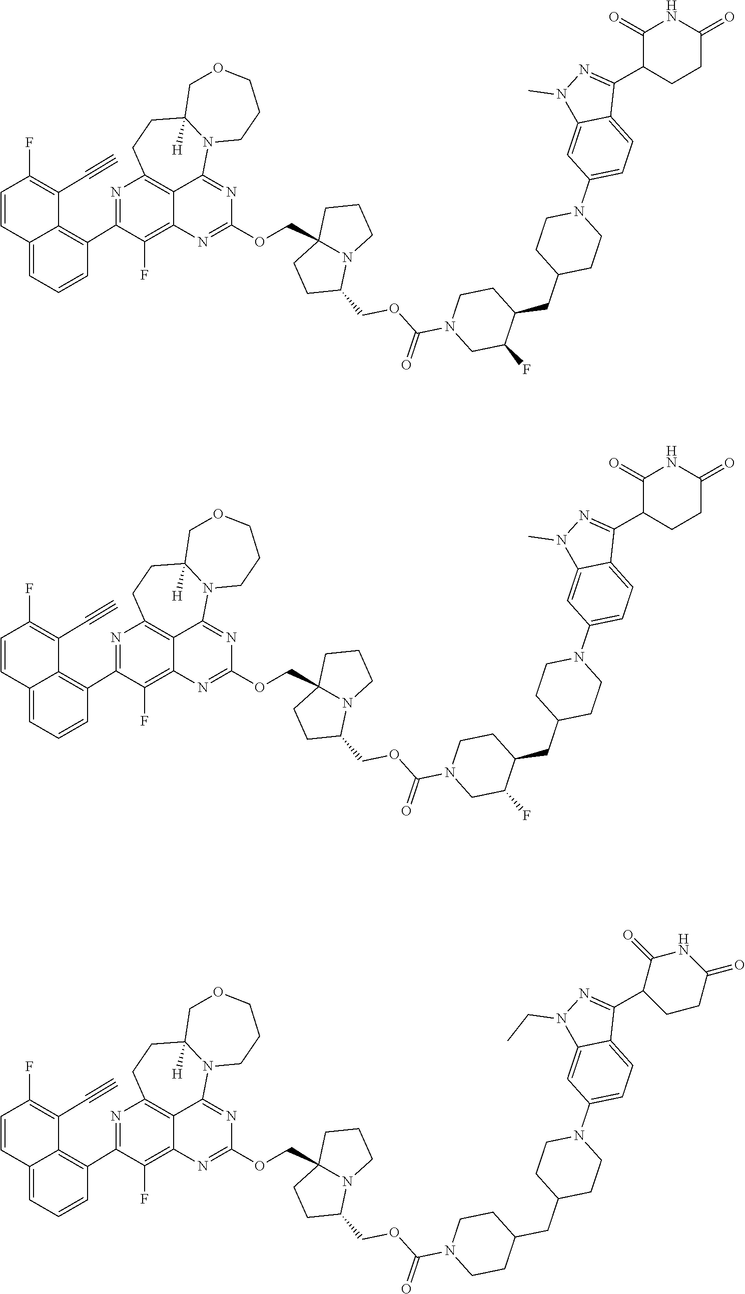

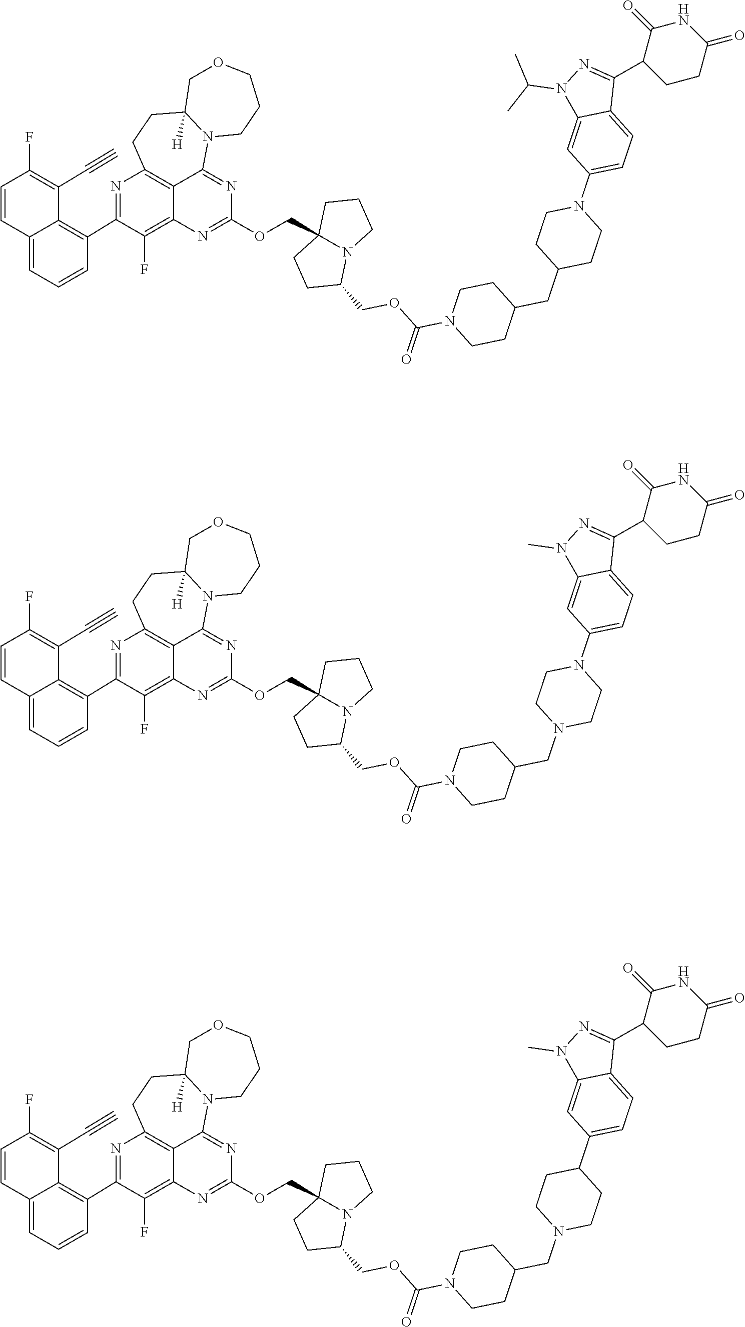

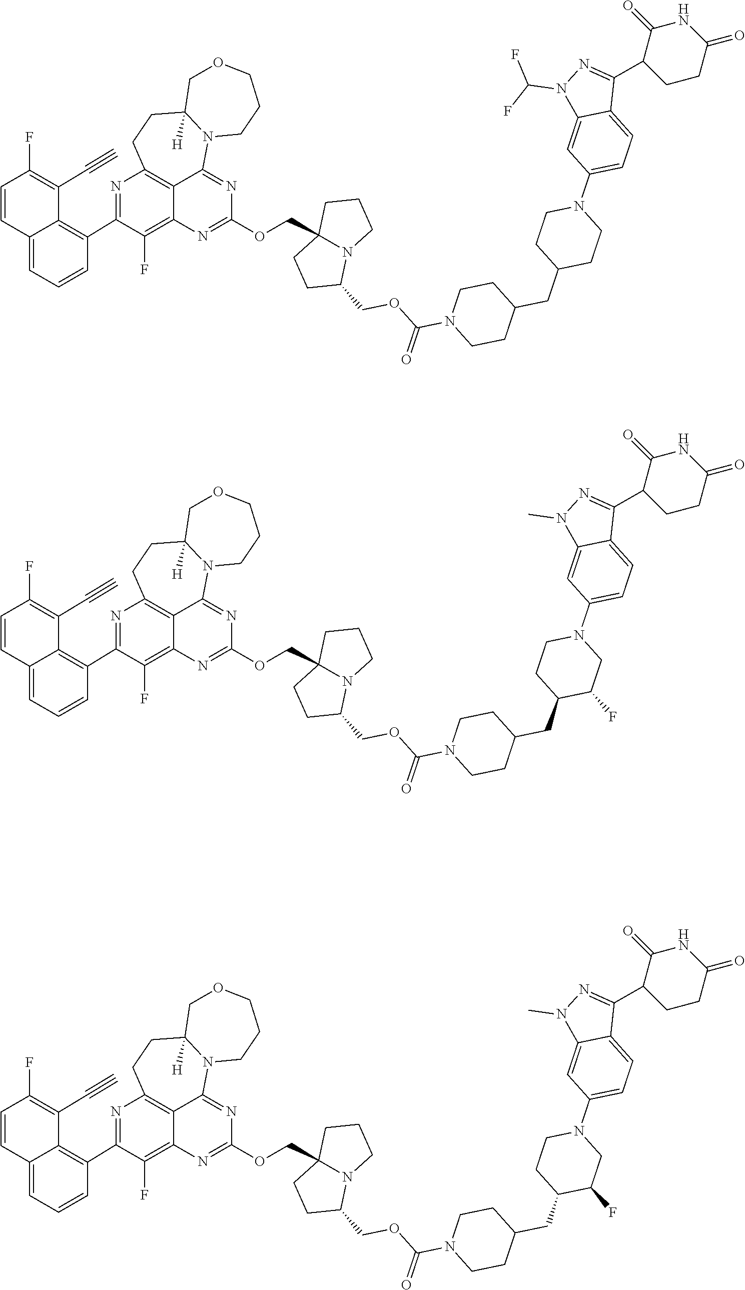

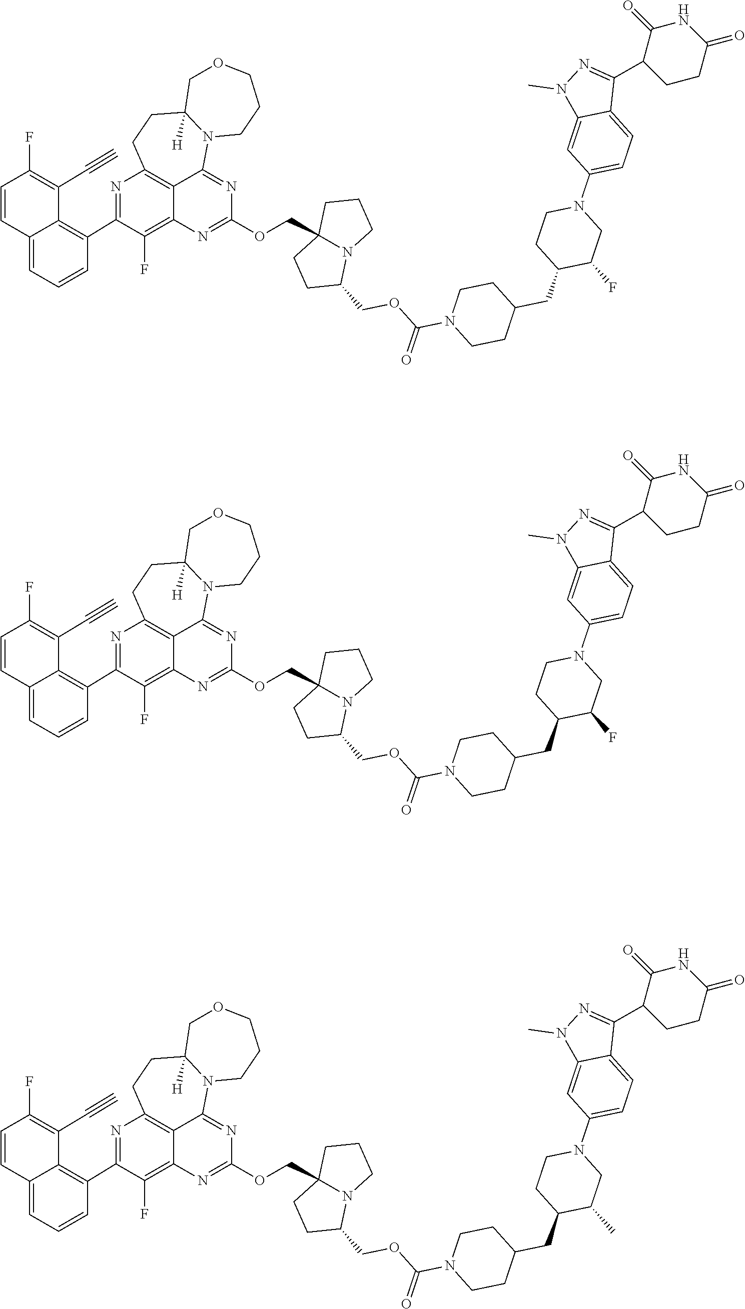

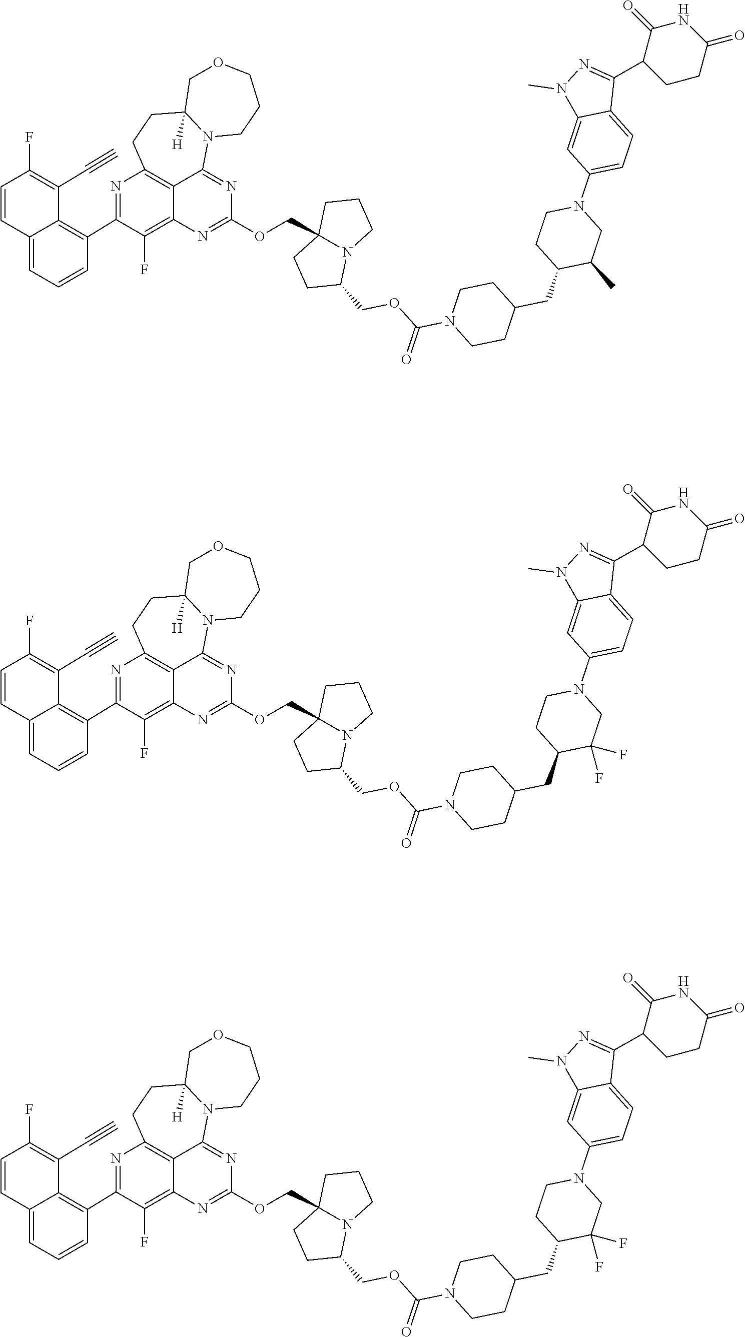

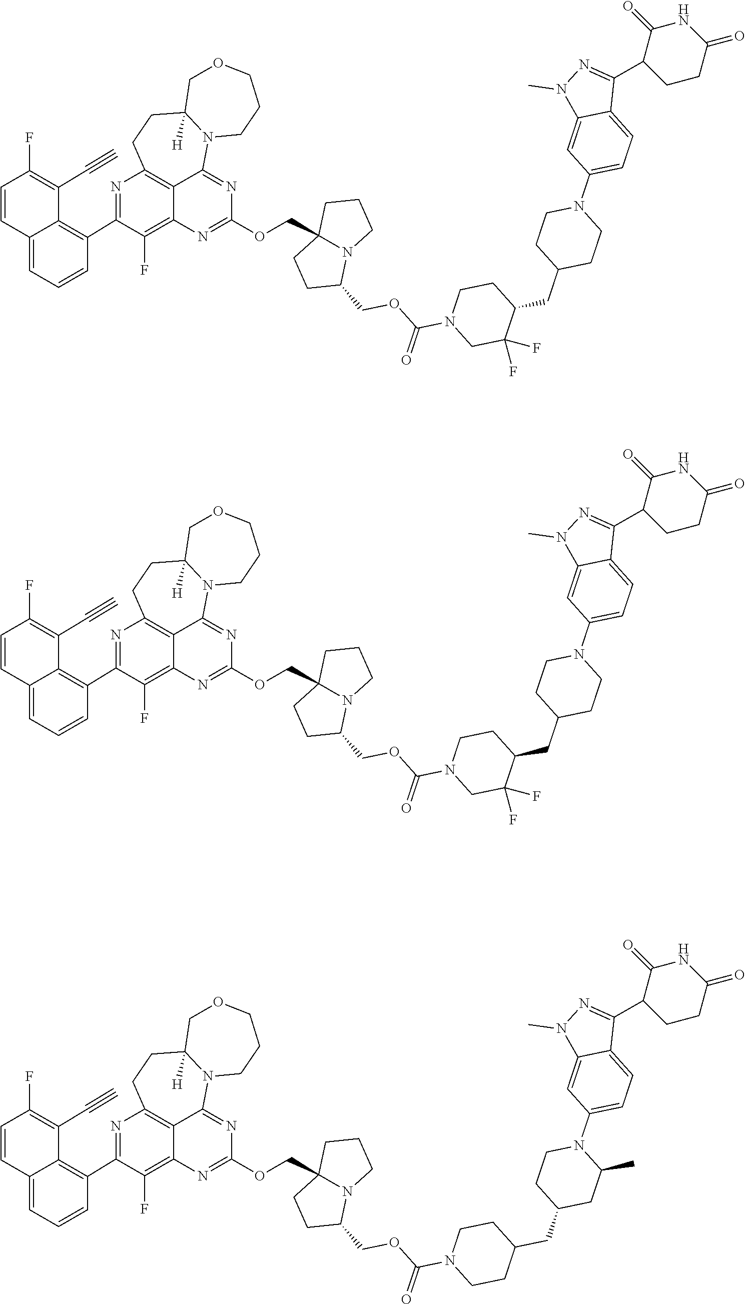

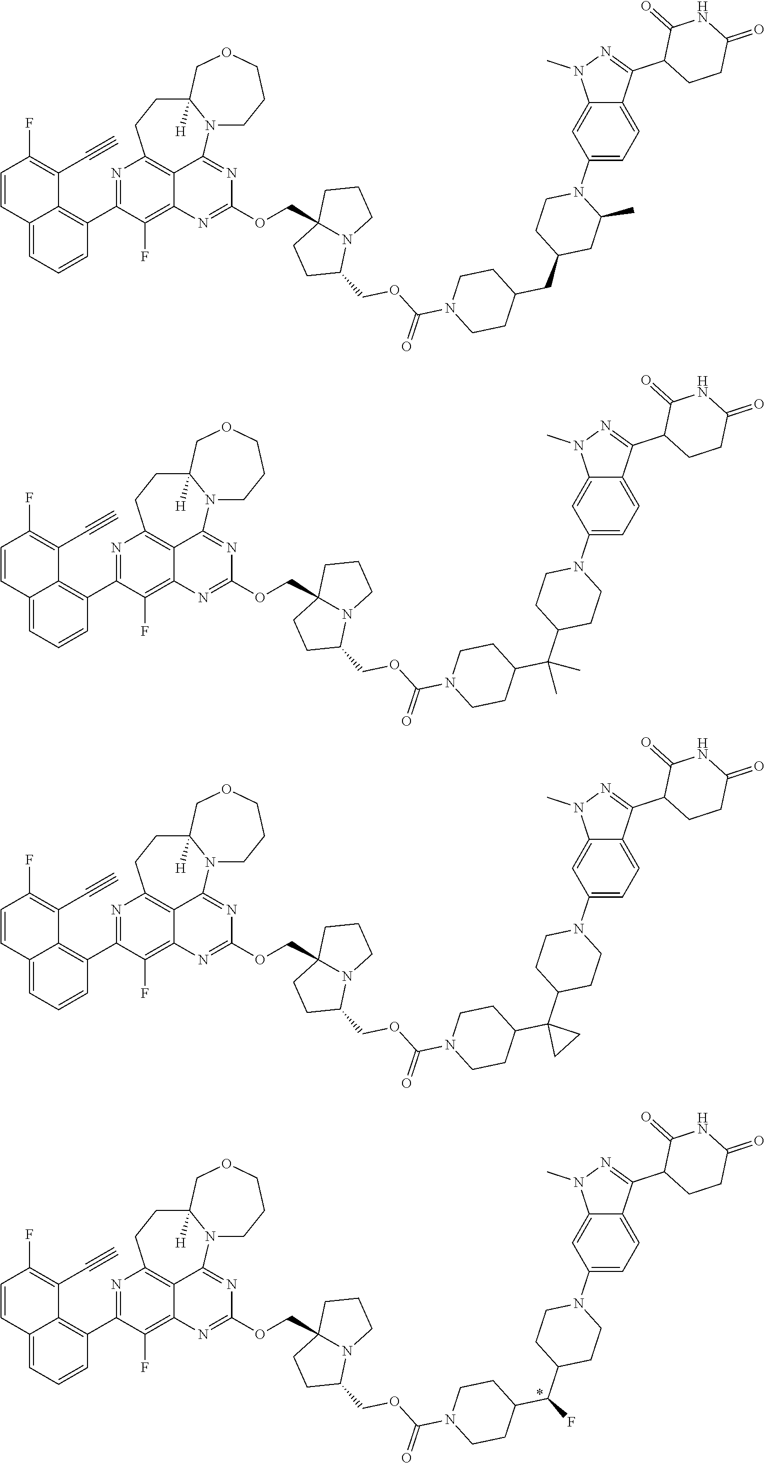

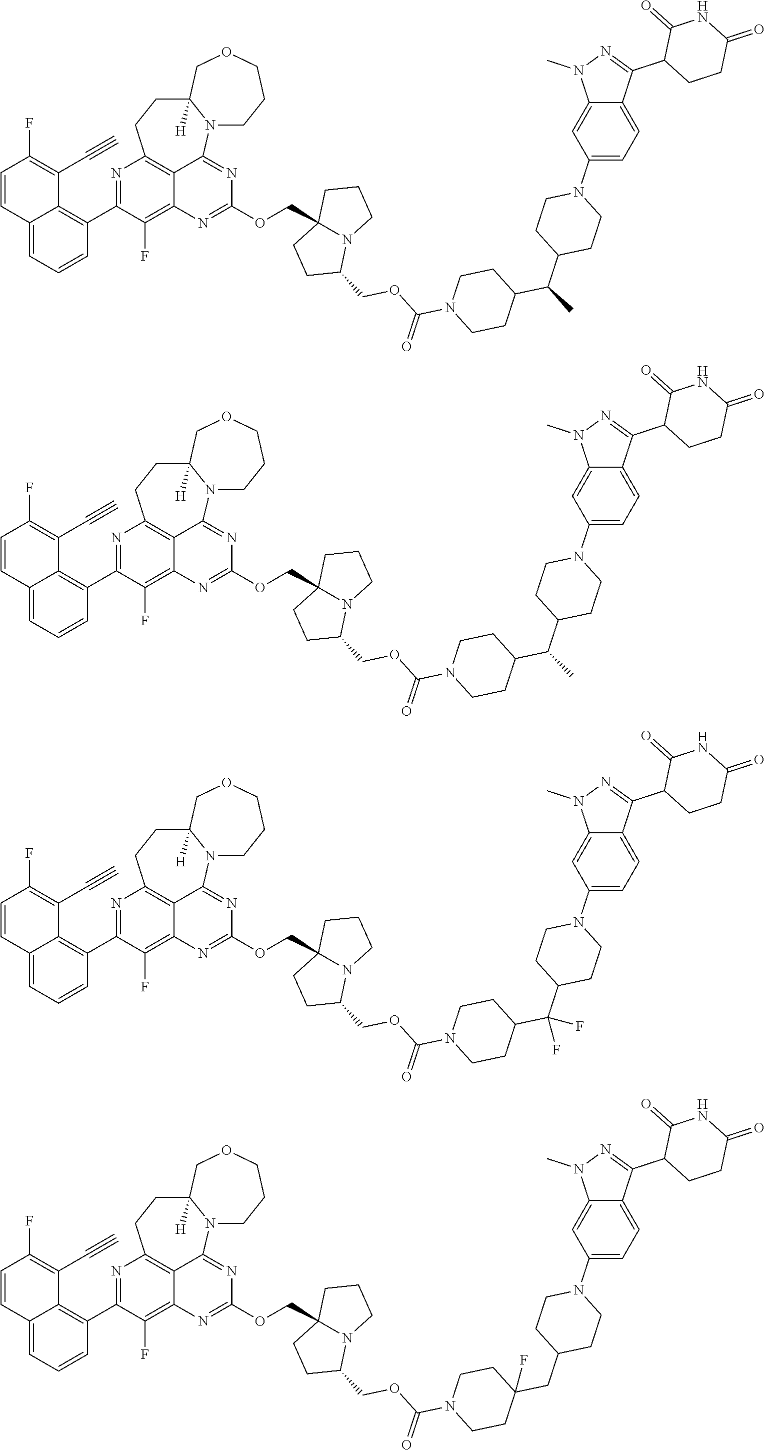

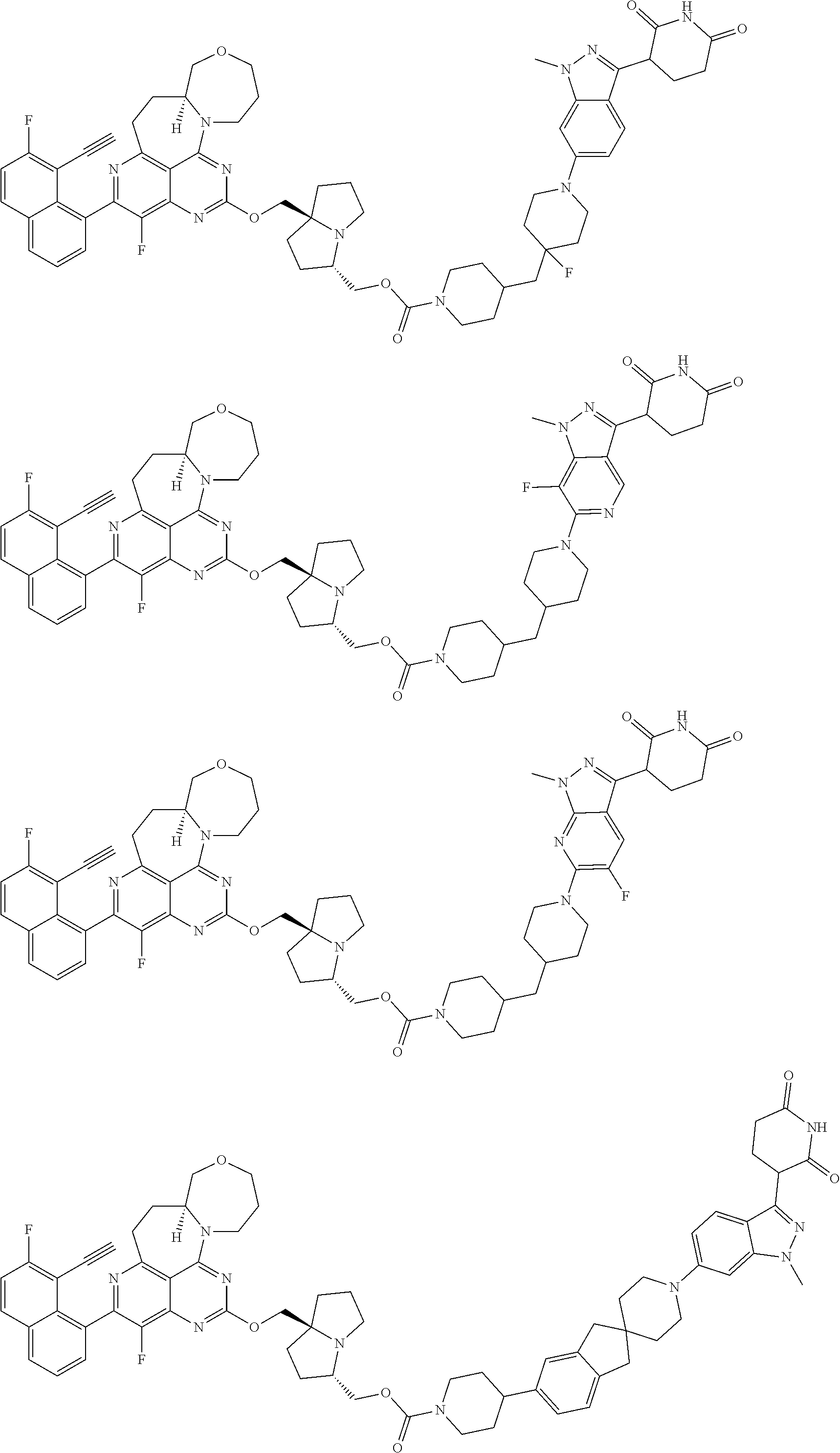

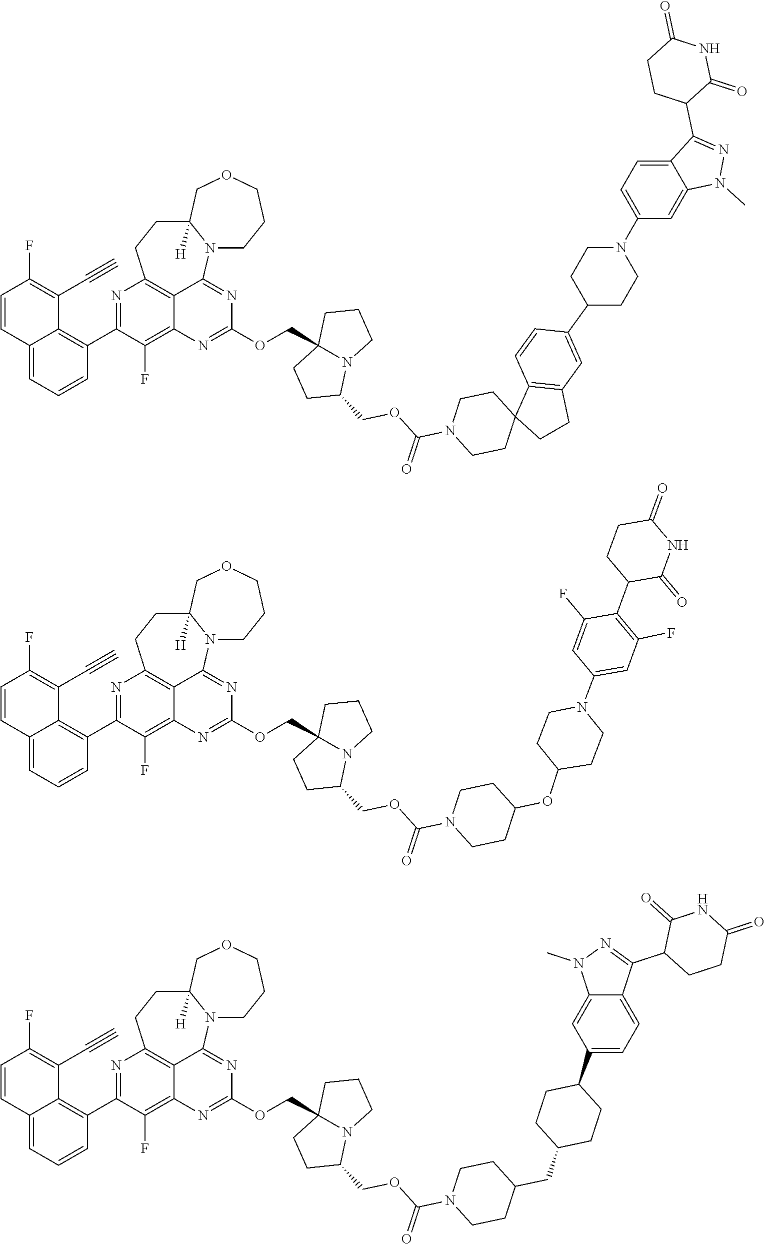

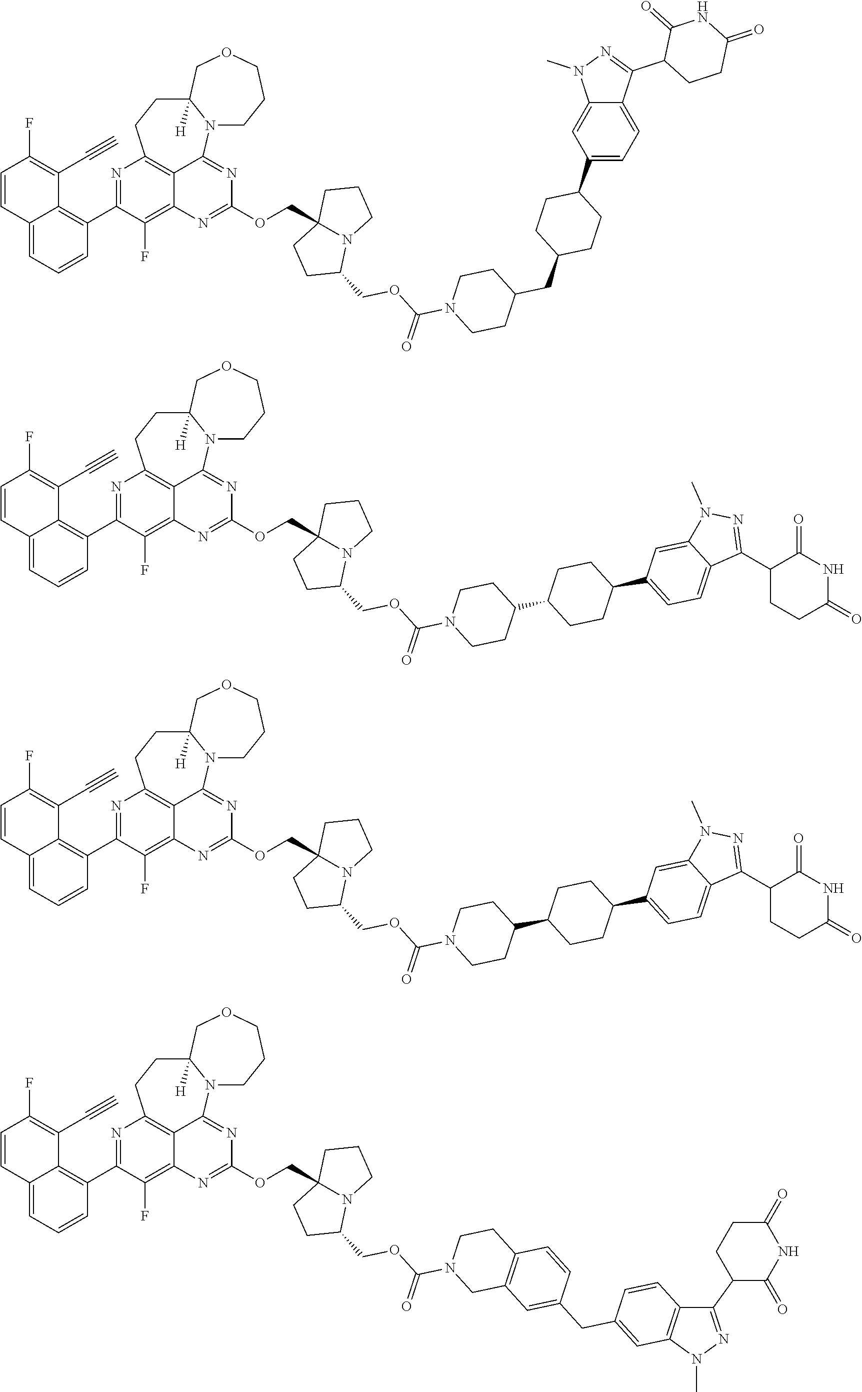

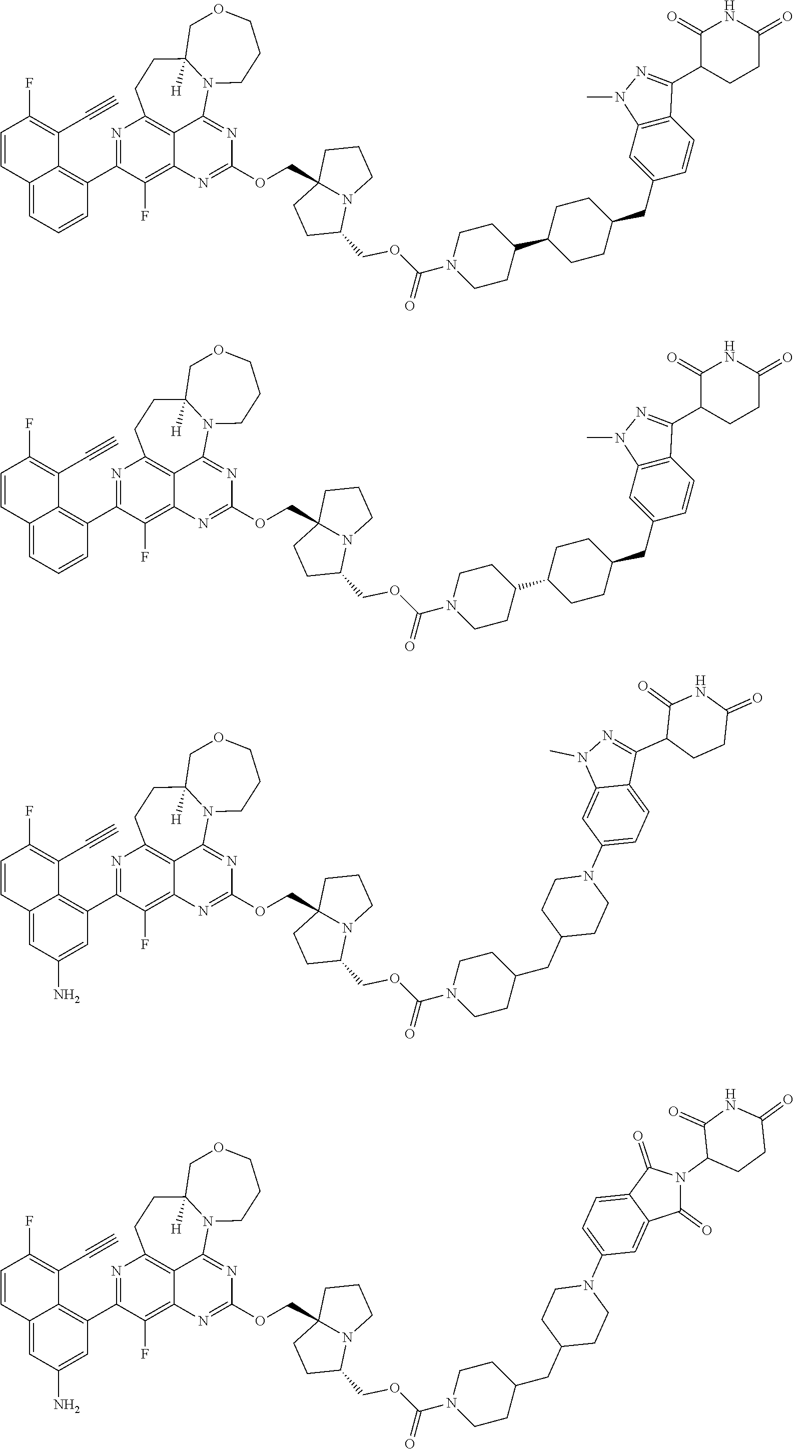

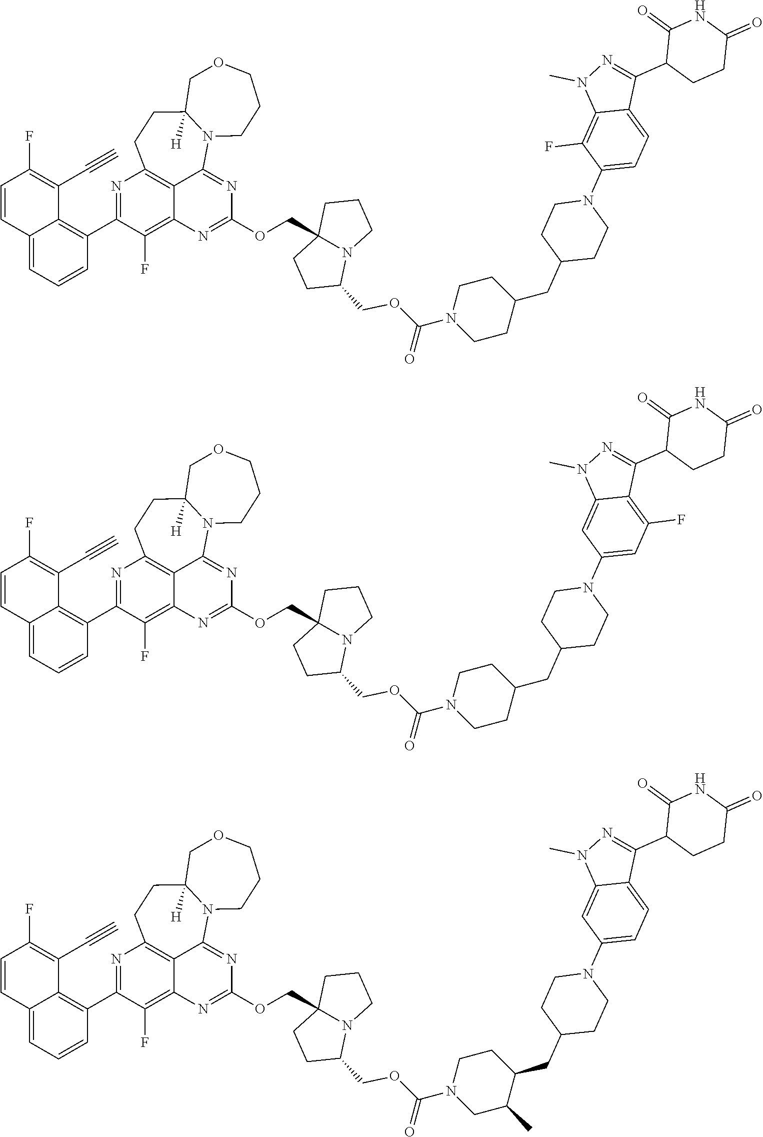

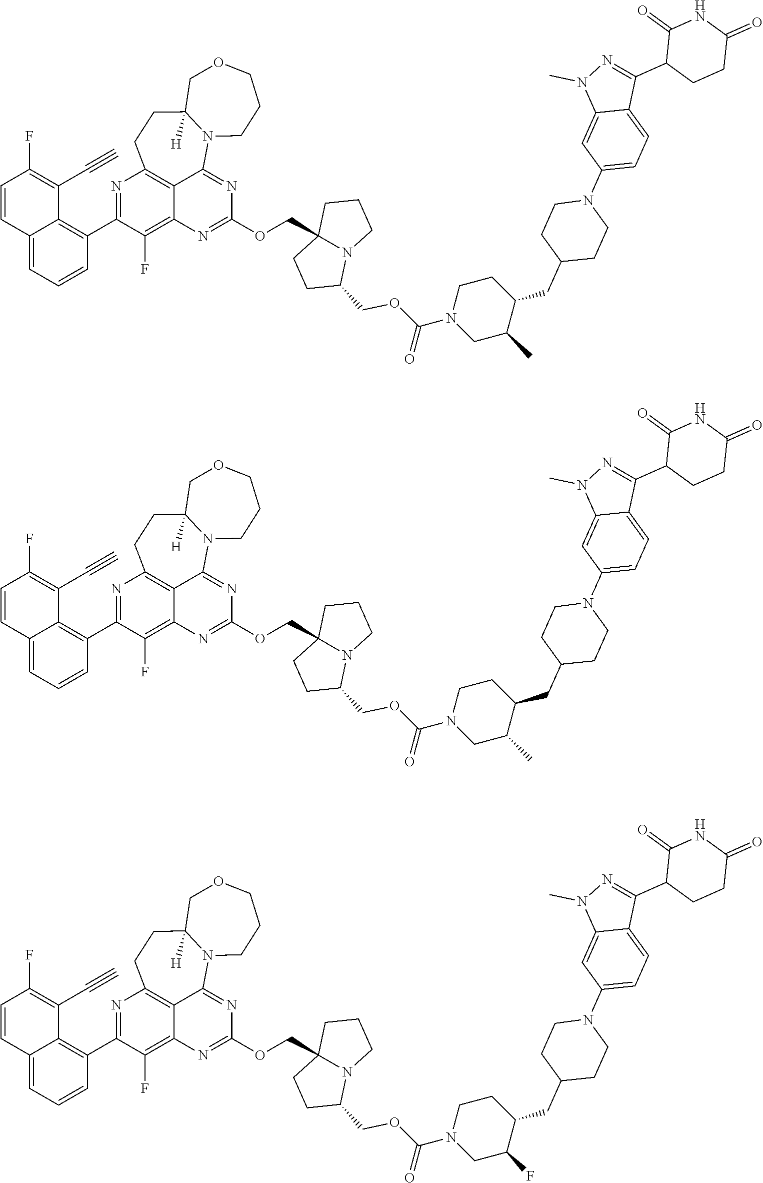

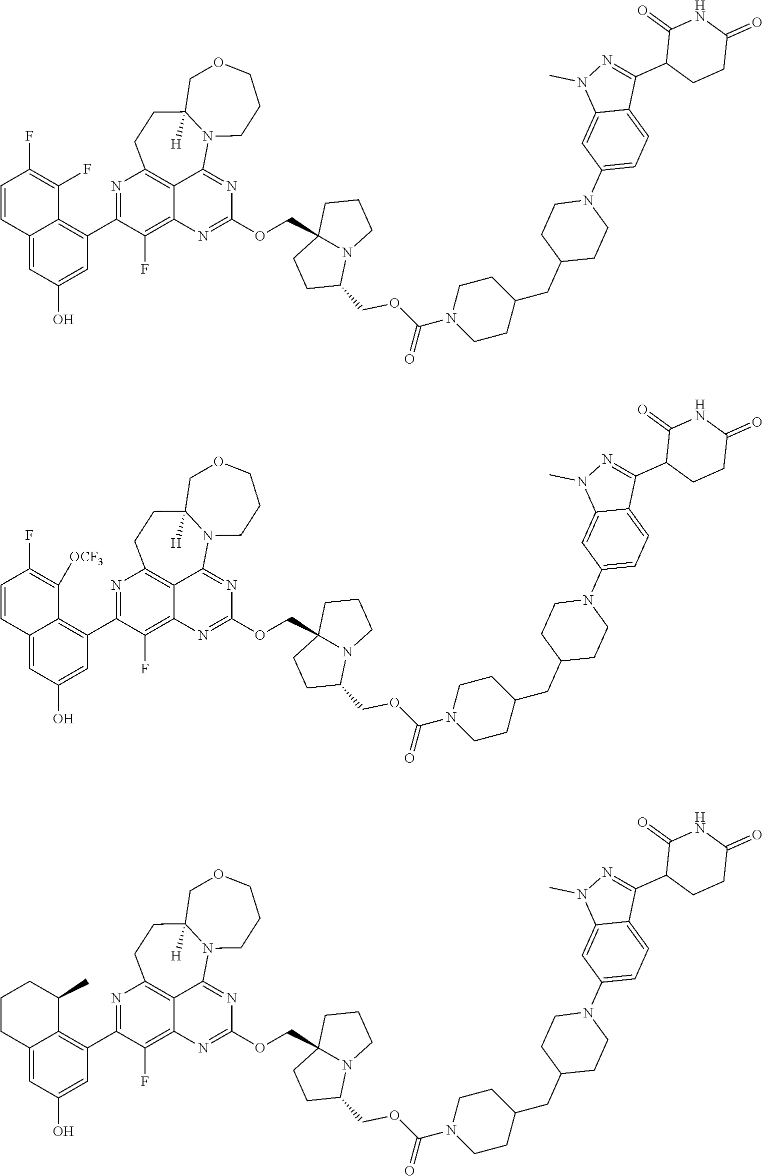

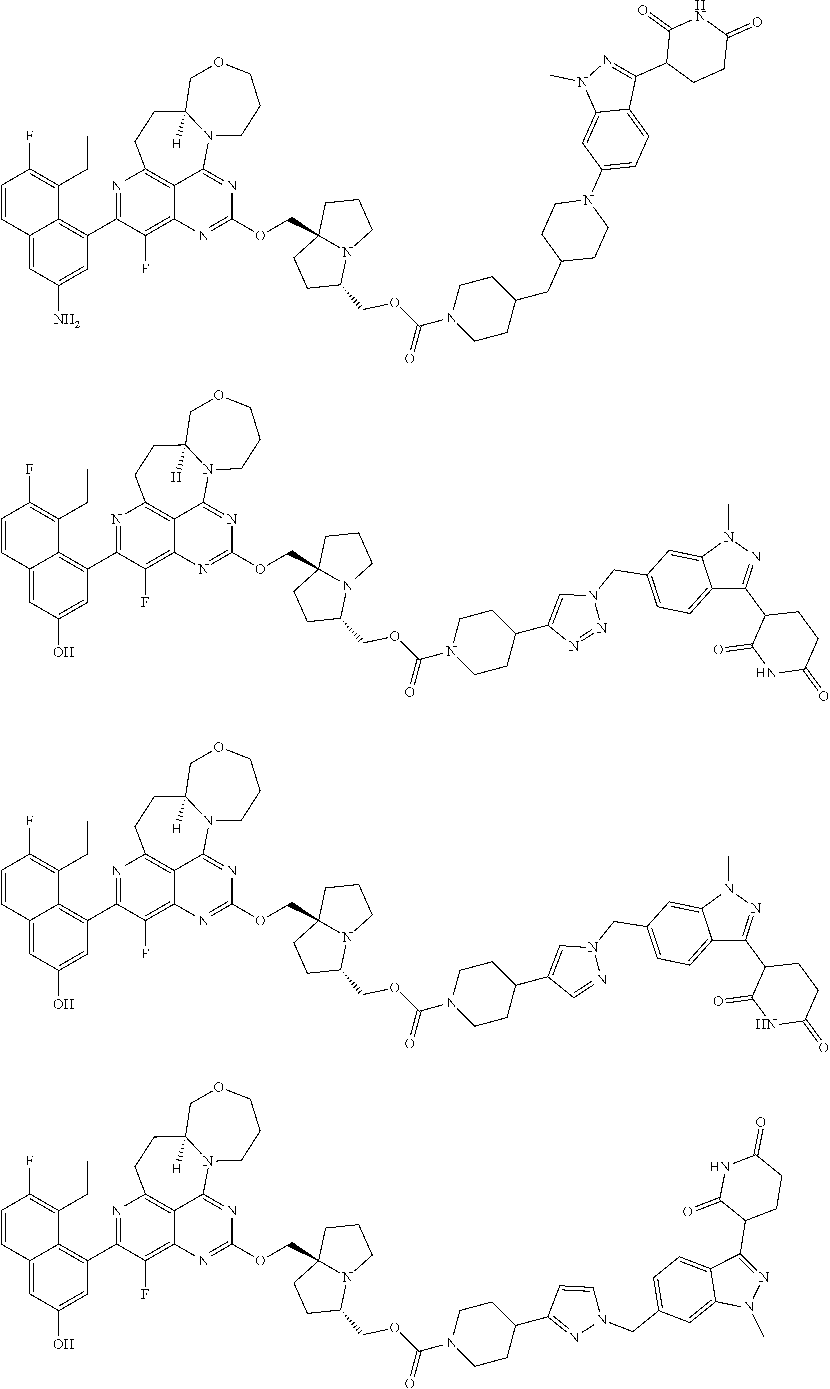

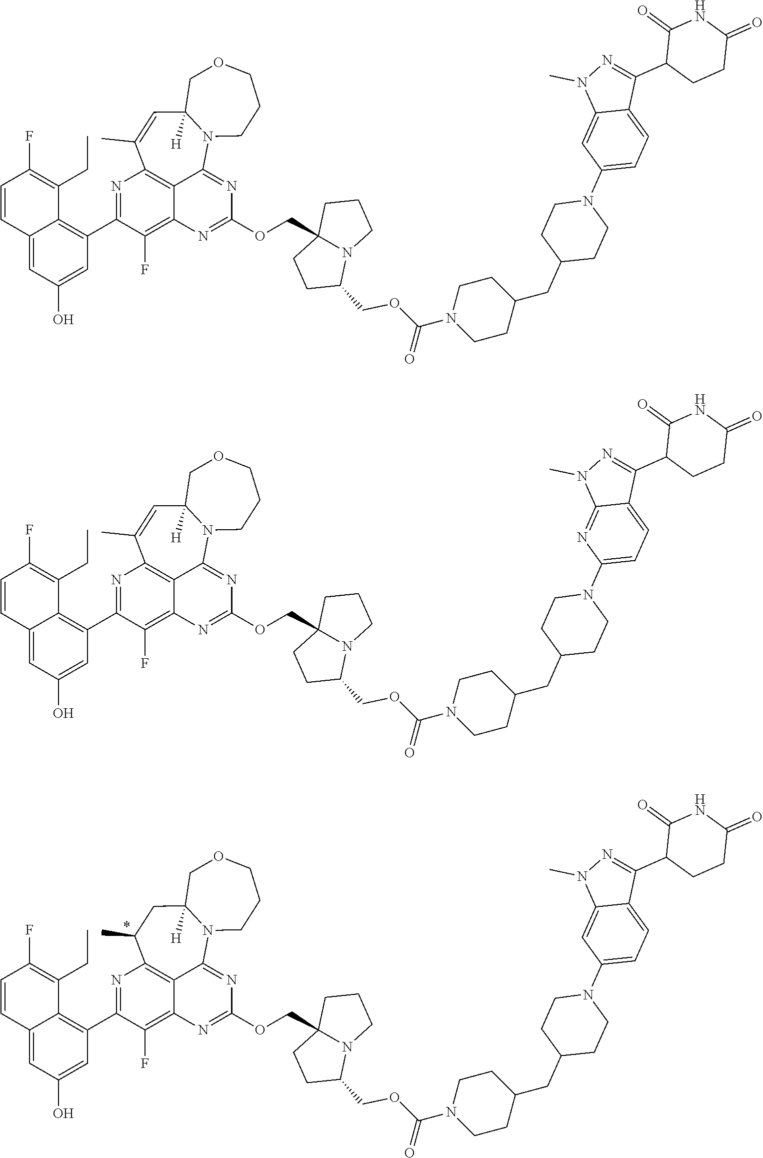

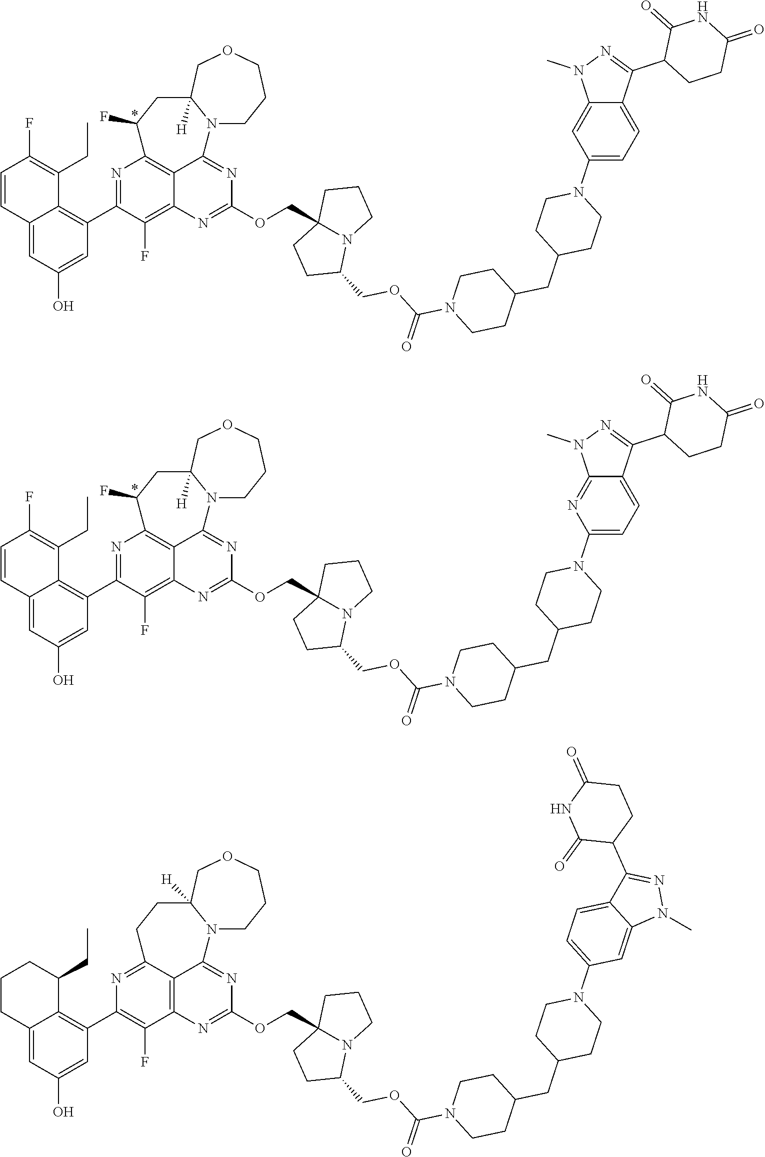

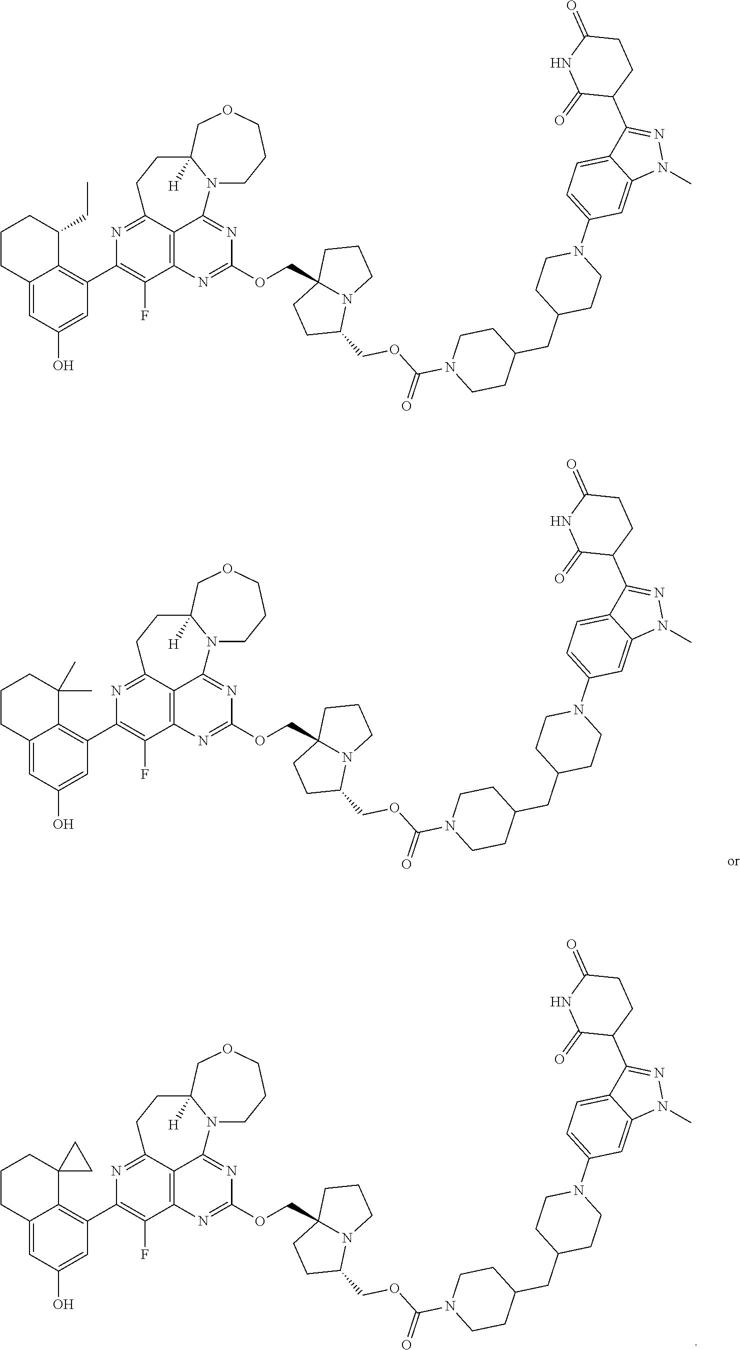

52. The compound of claim 1, or pharmaceutically acceptable salt thereof, wherein the compound has the structure:

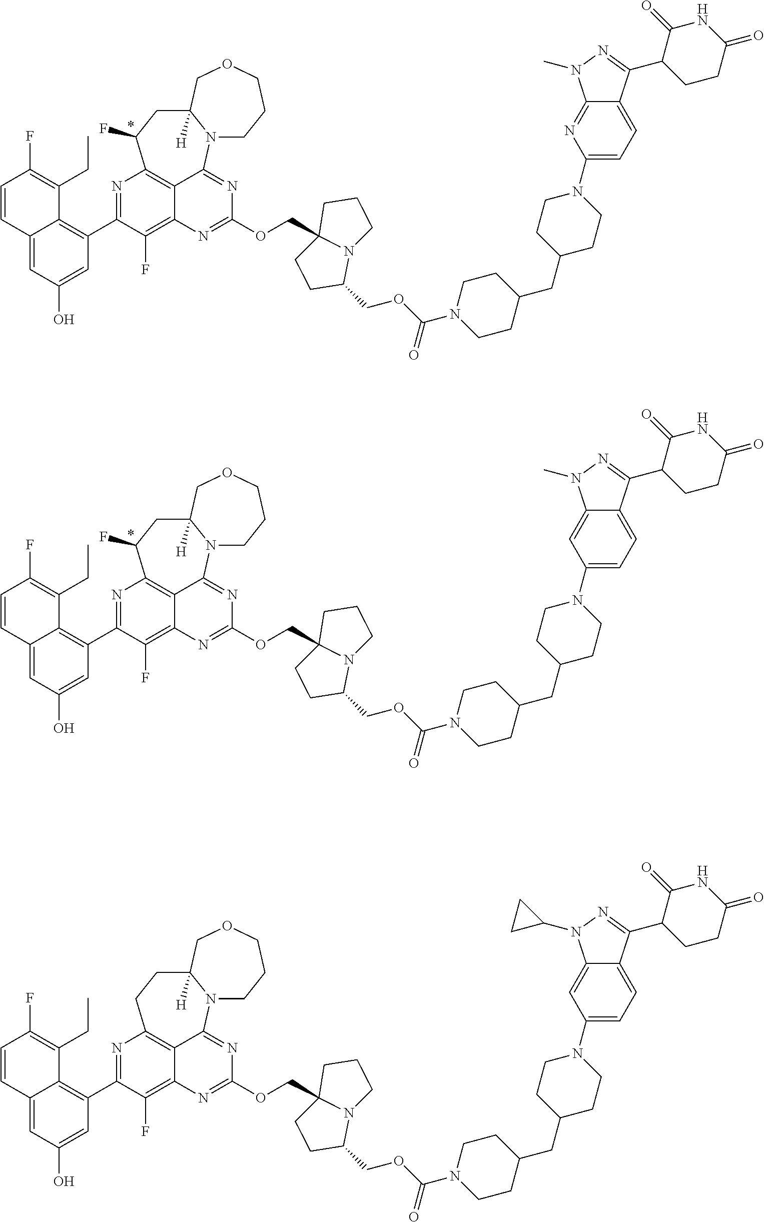

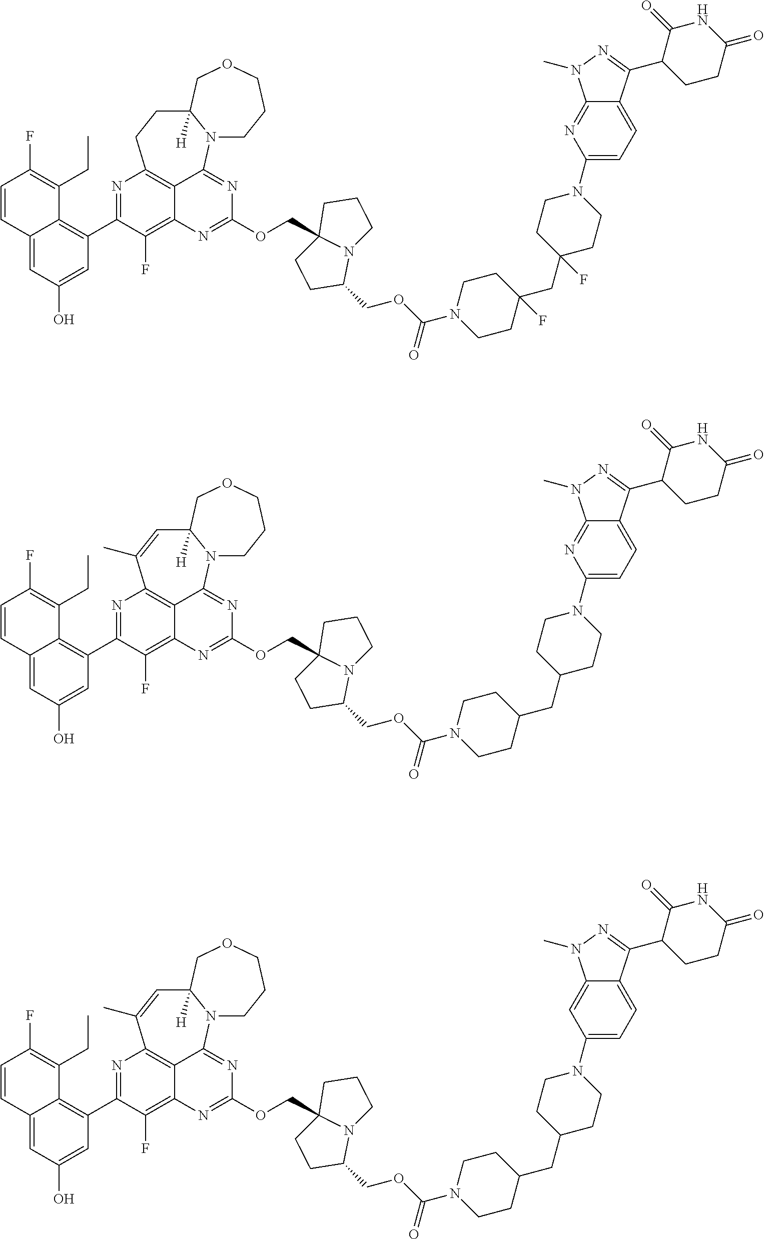

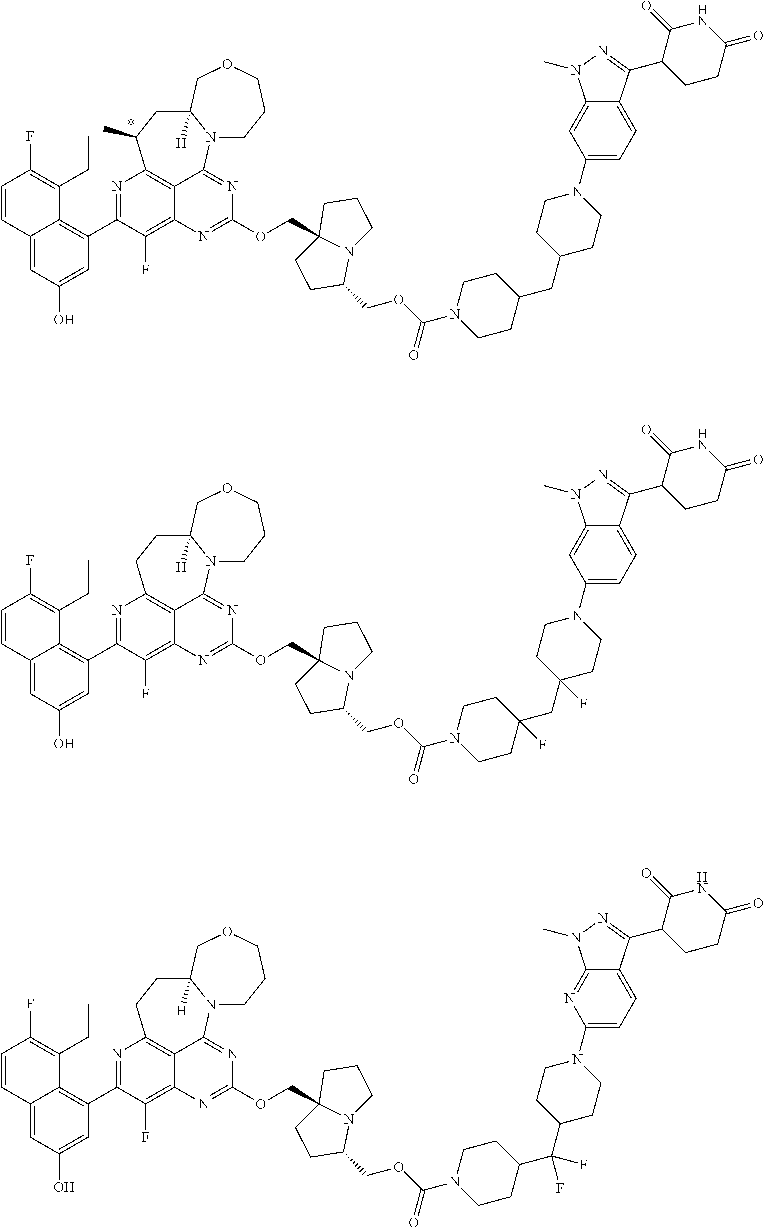

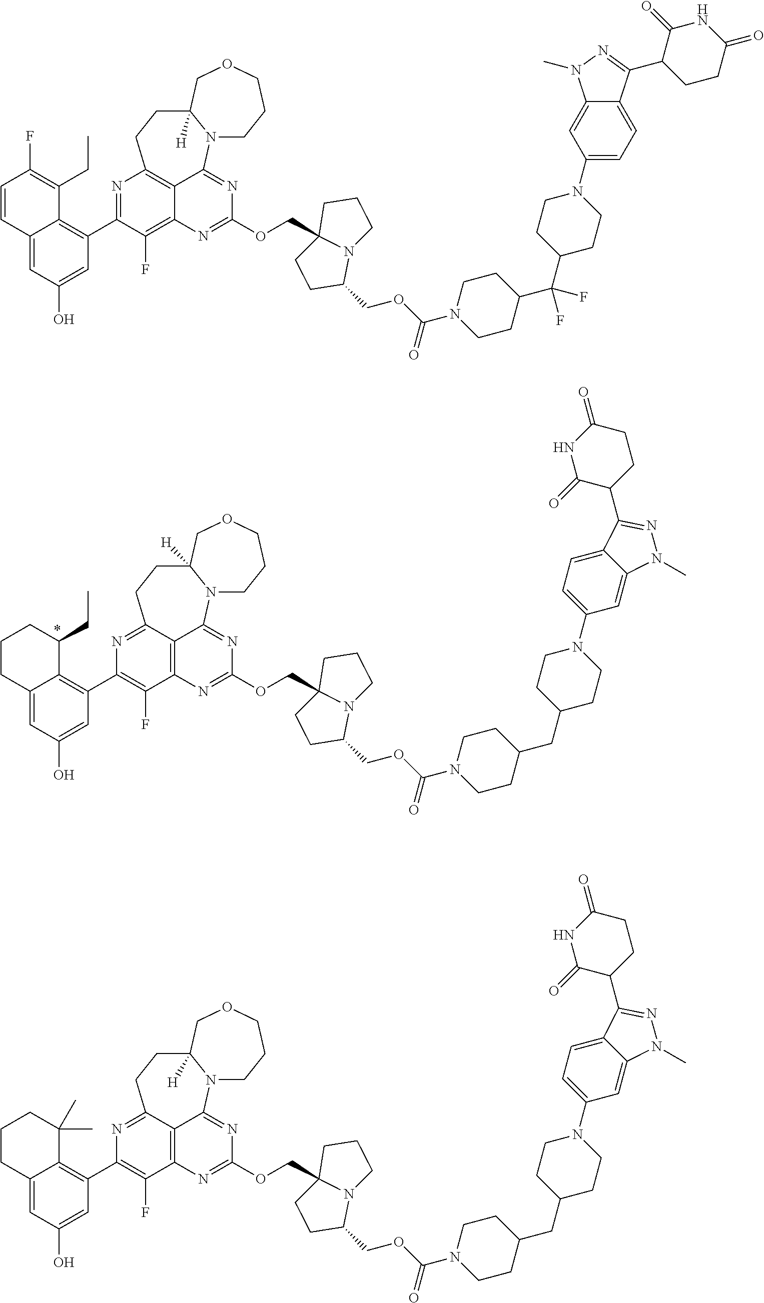

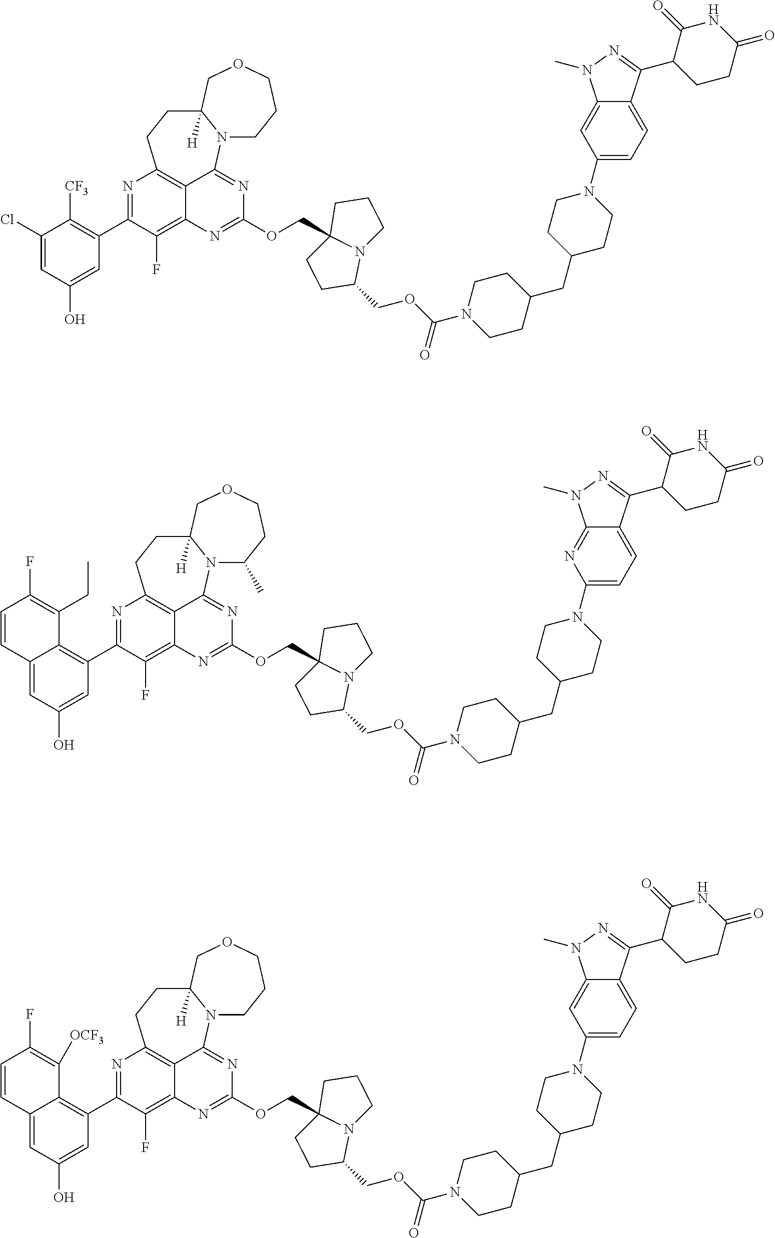

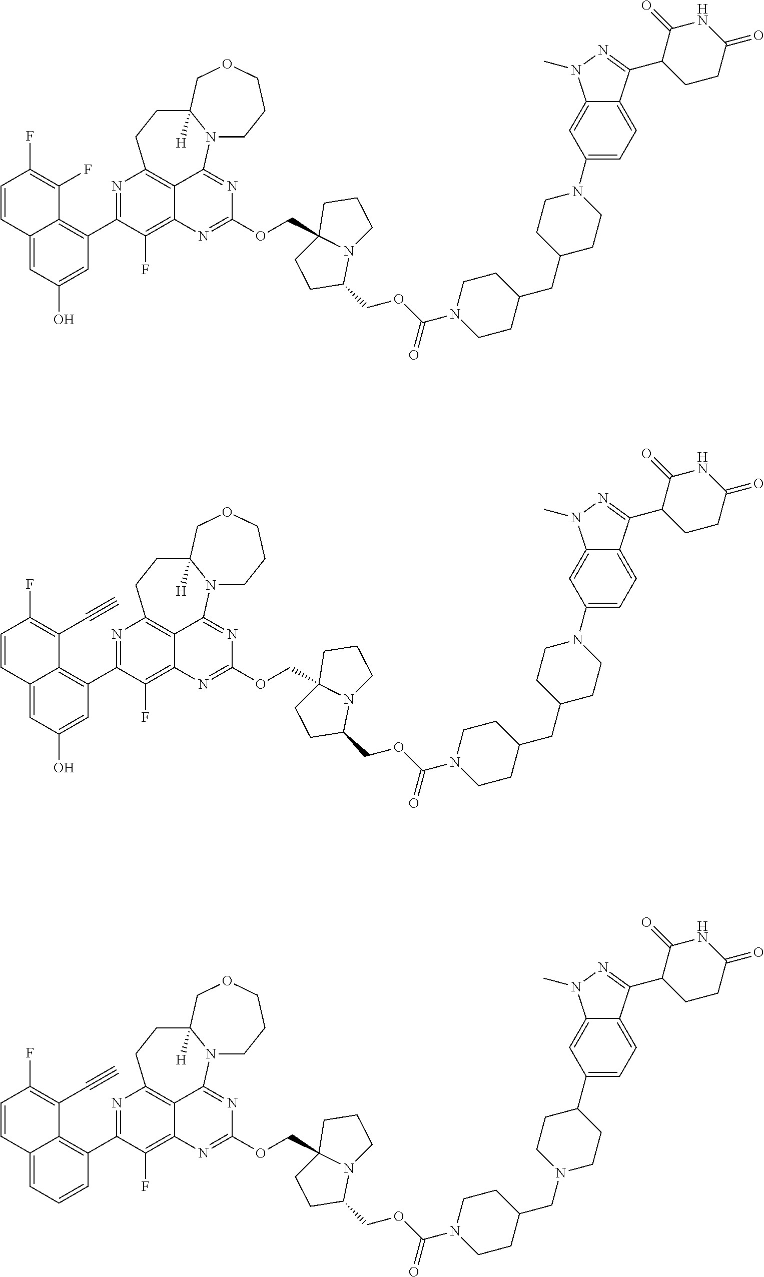

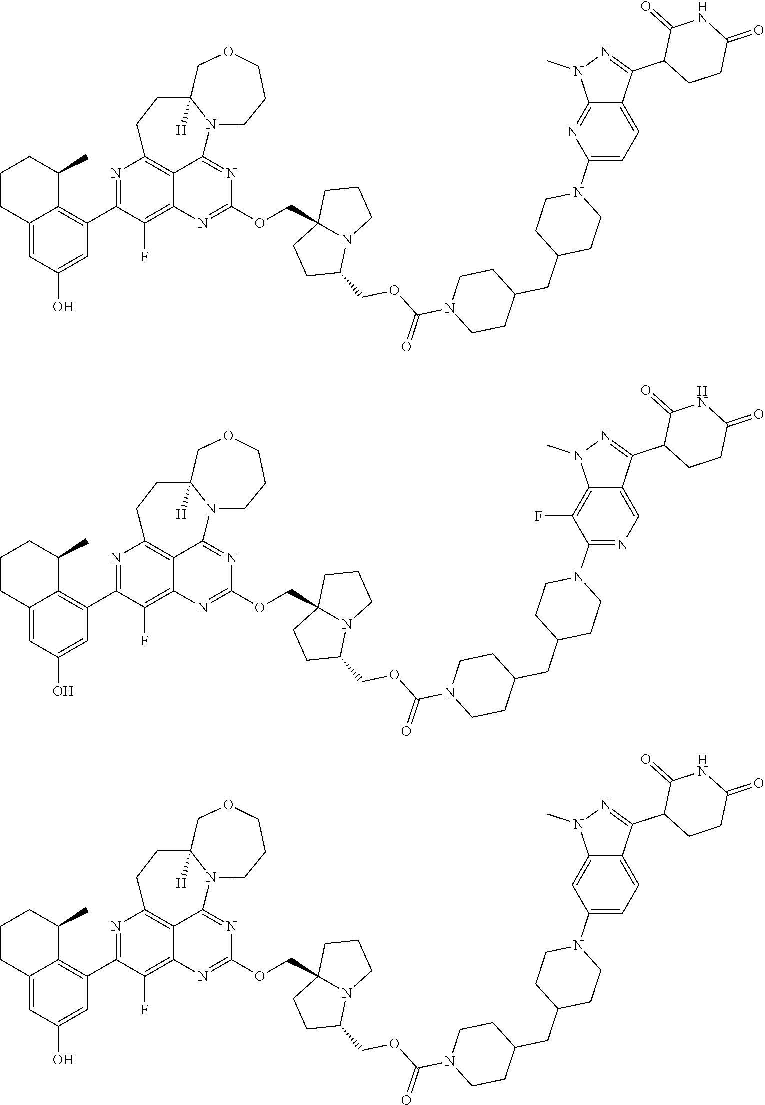

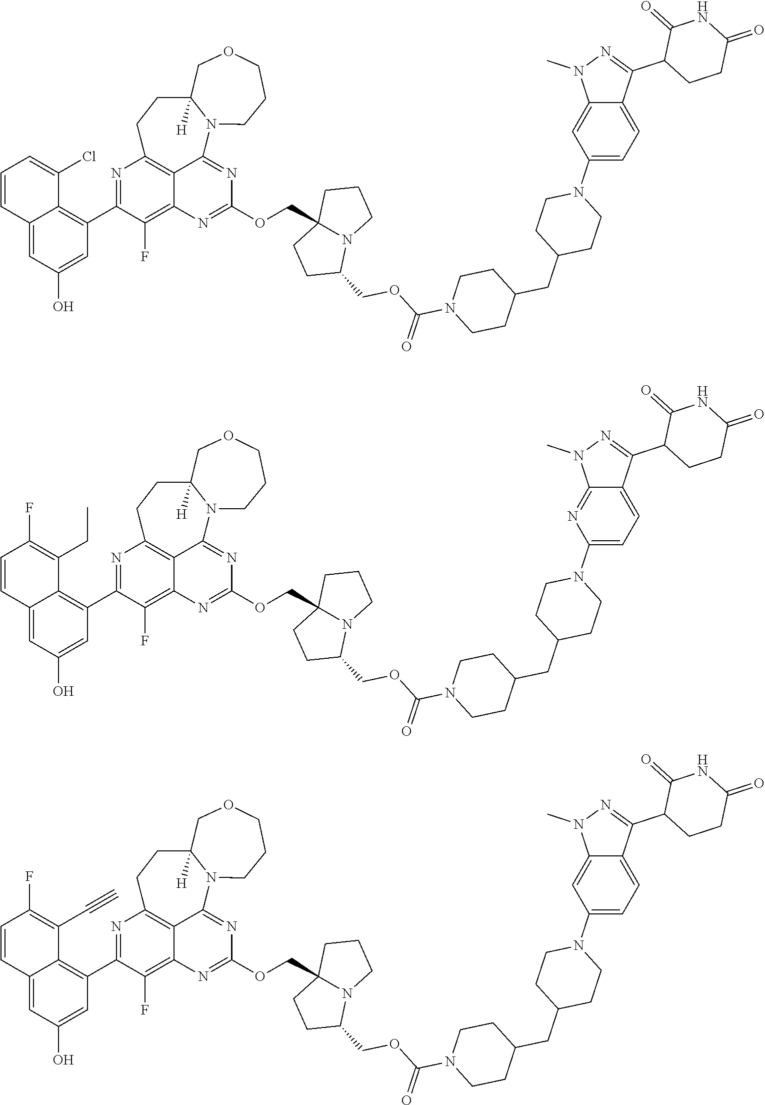

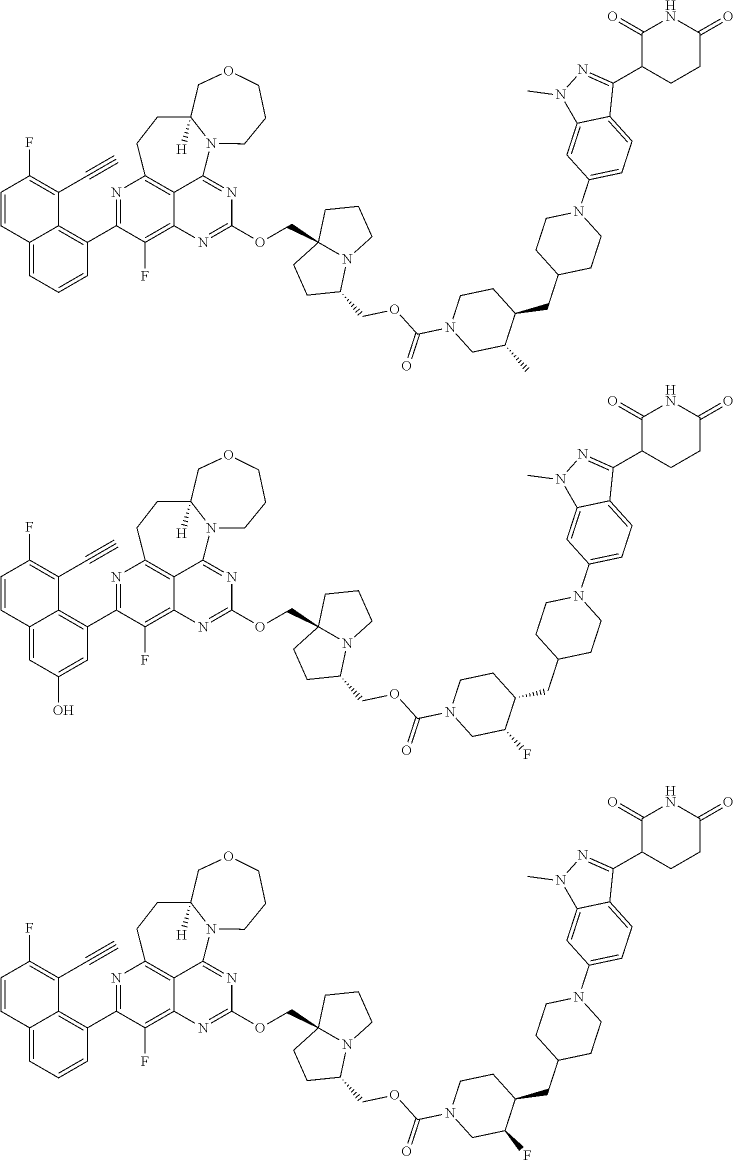

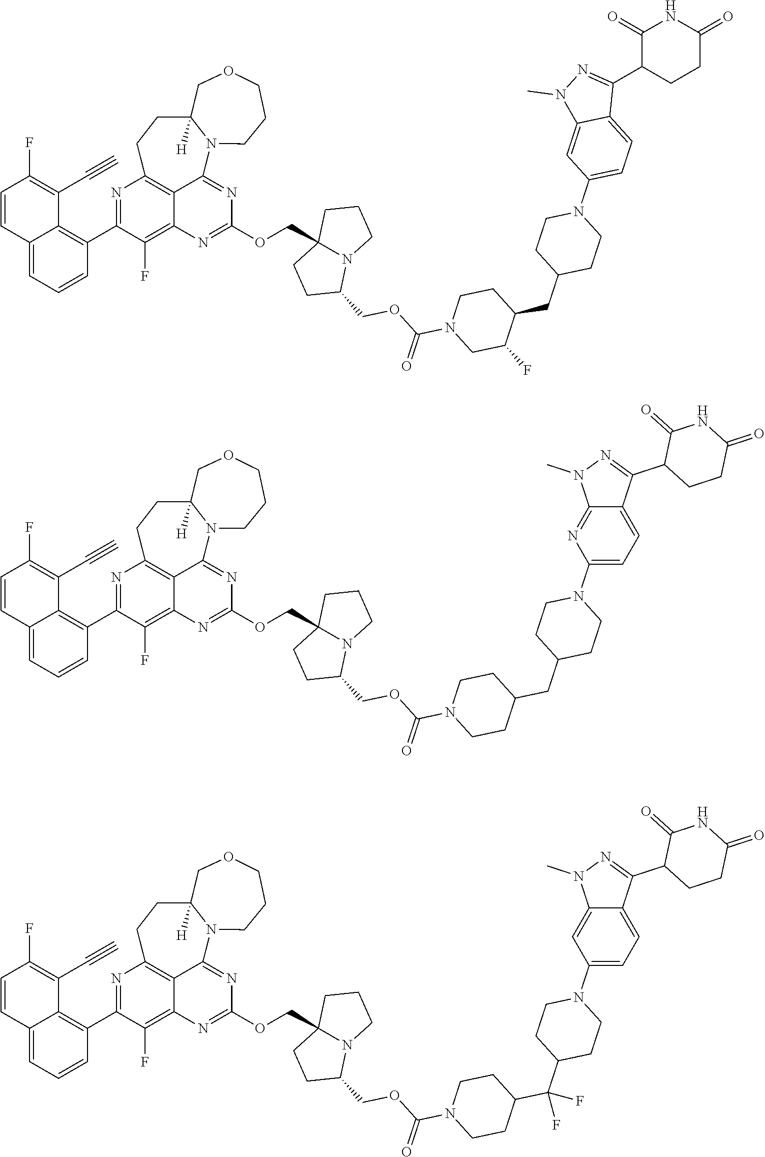

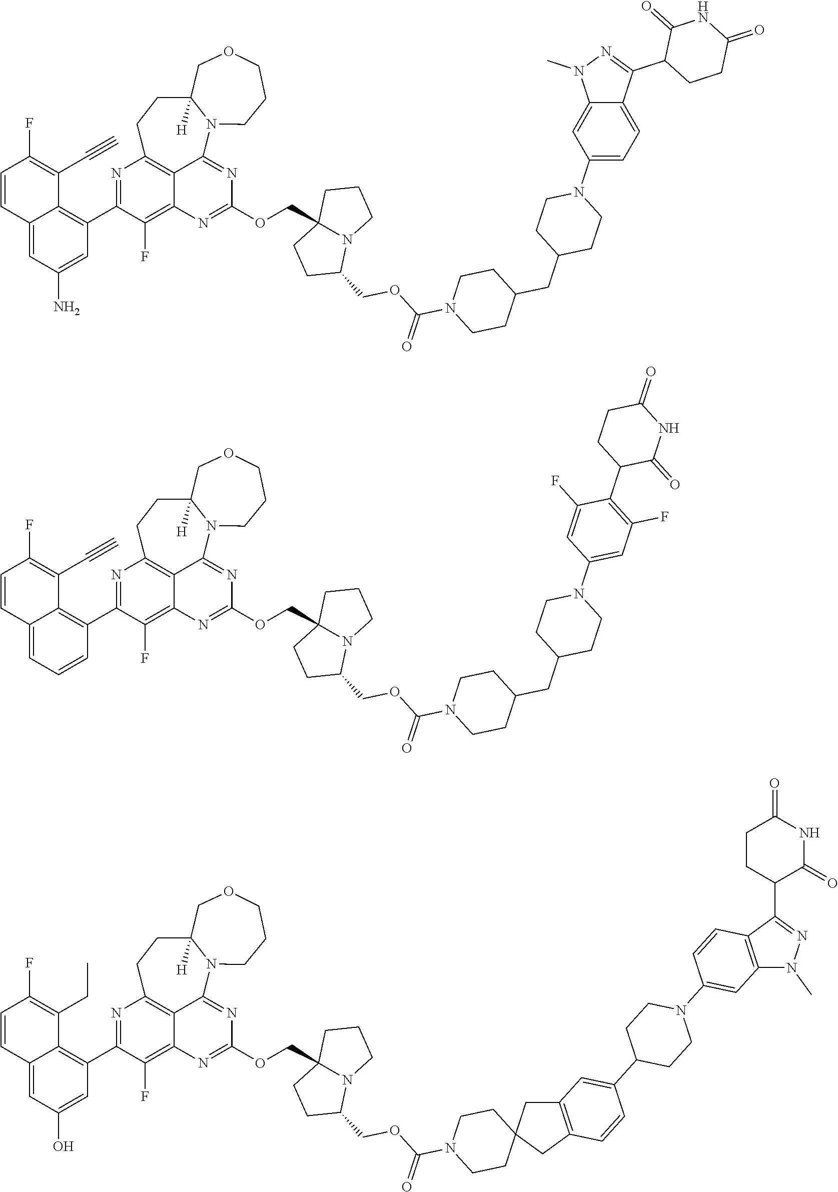

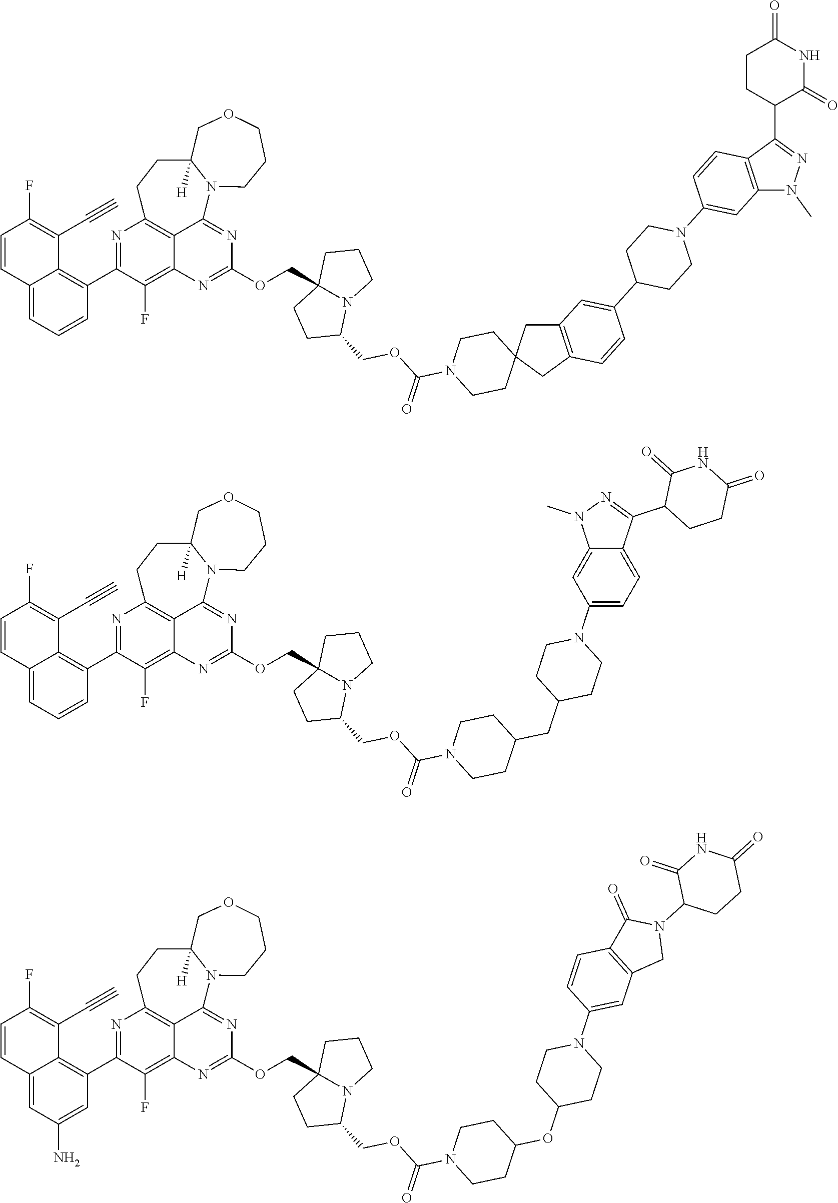

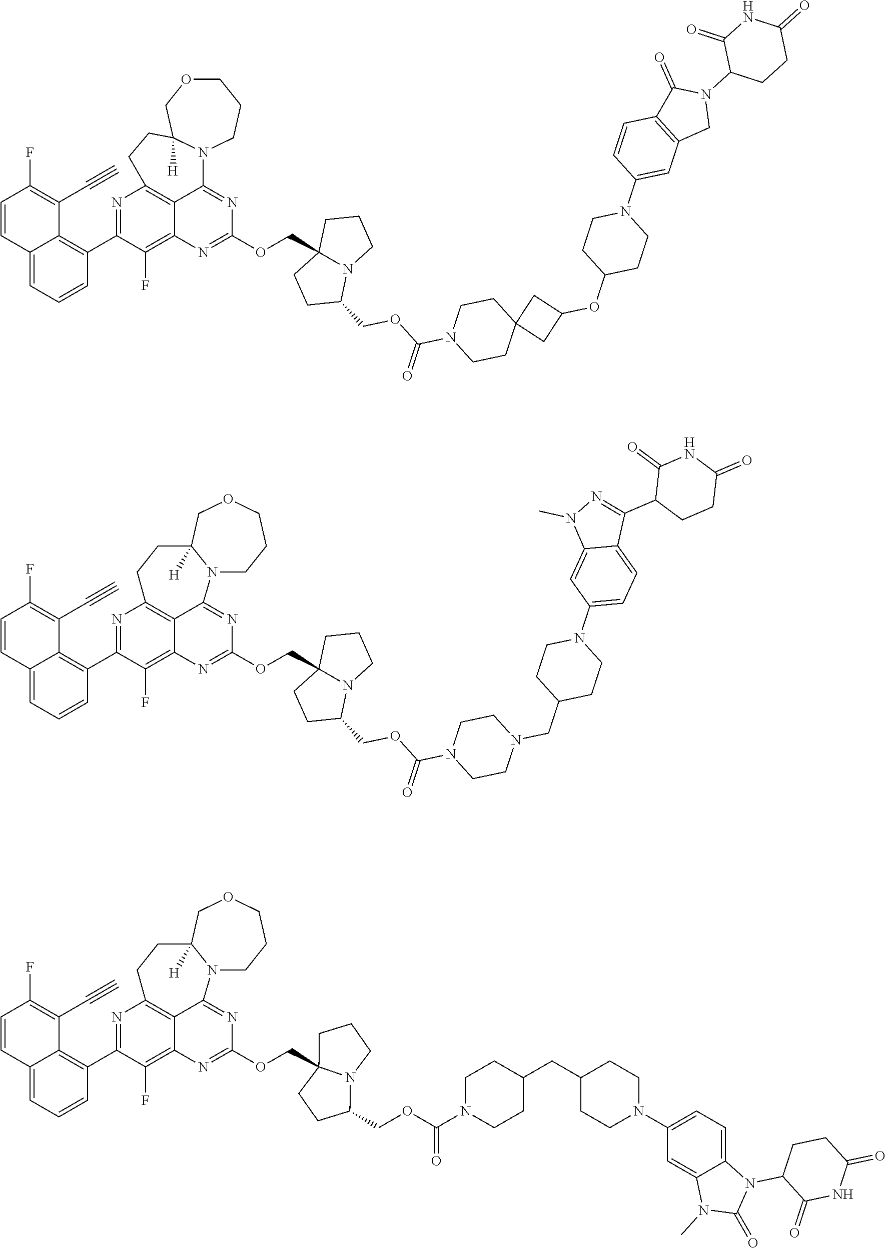

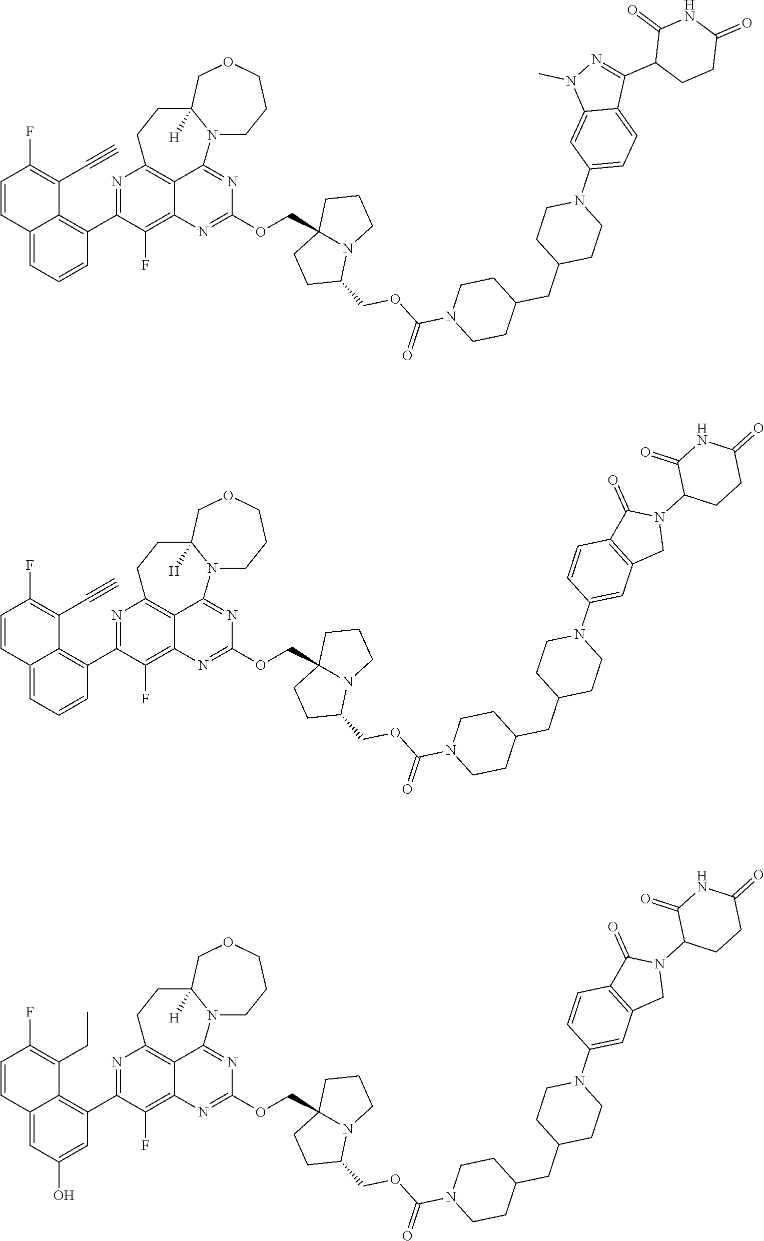

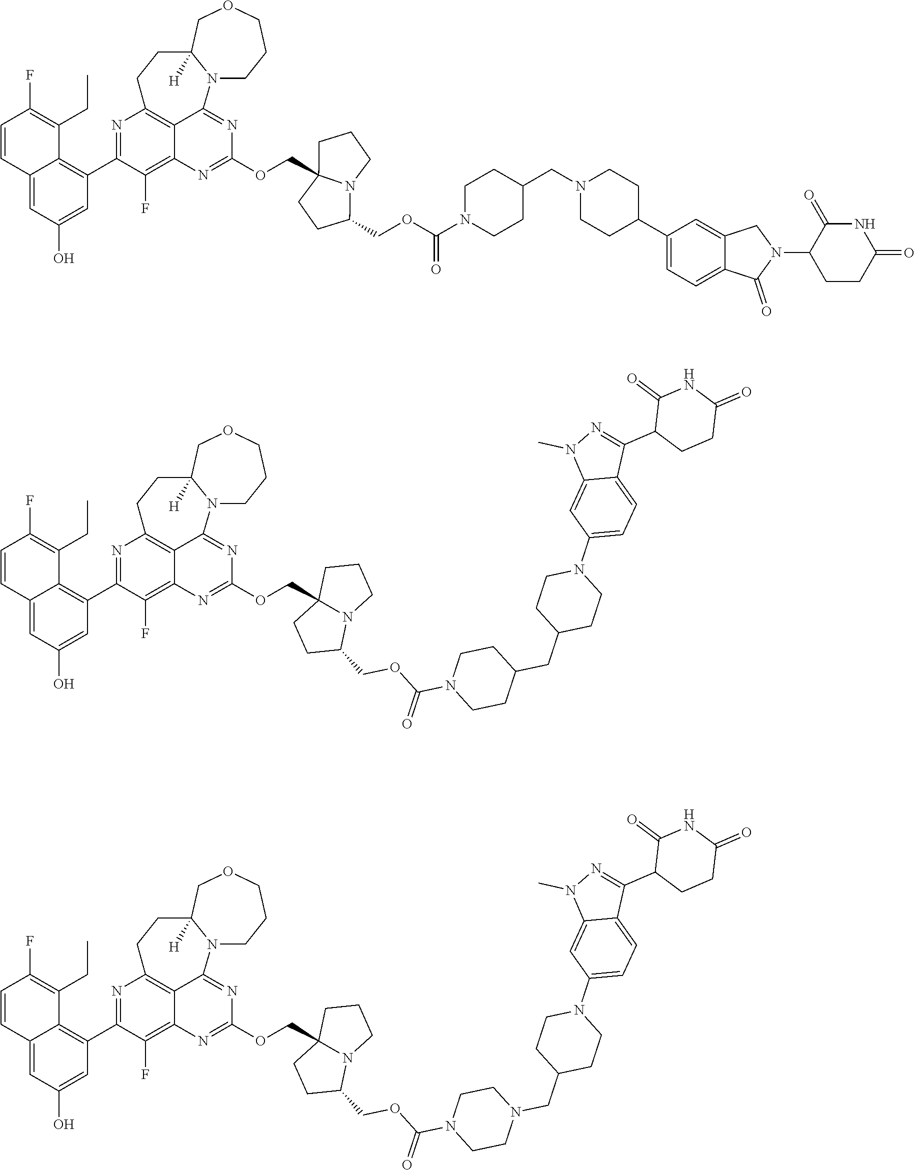

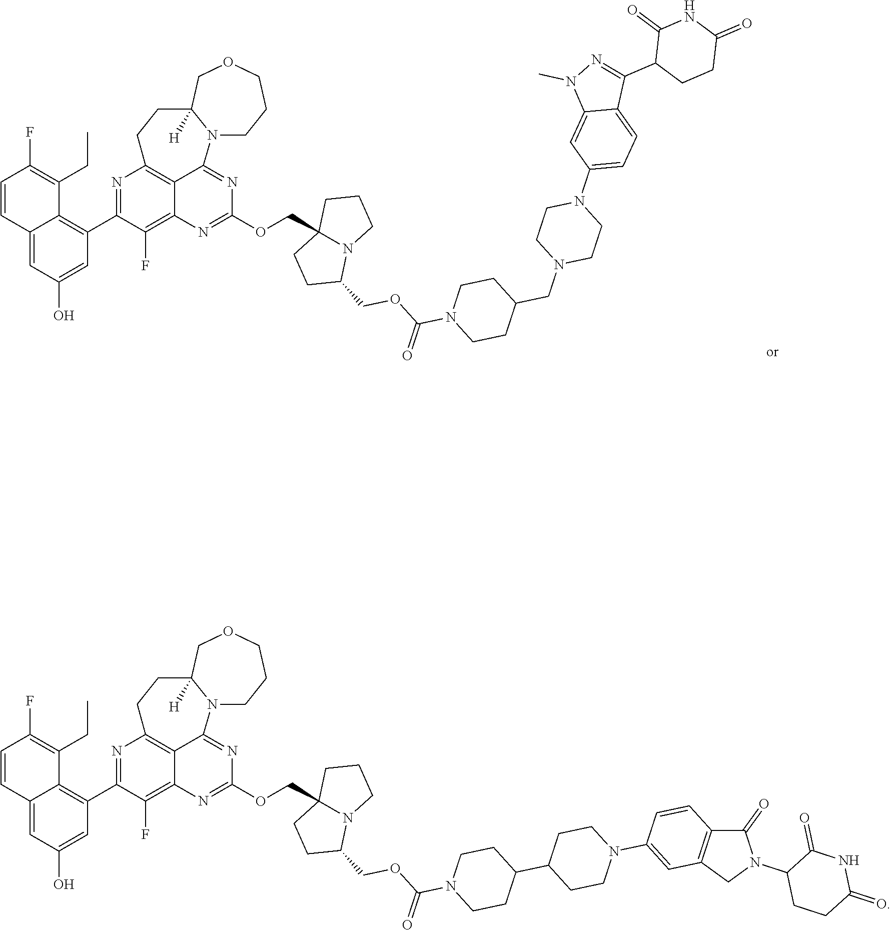

53. The compound of claim 1, or pharmaceutically acceptable salt thereof, wherein the compound has the structure:

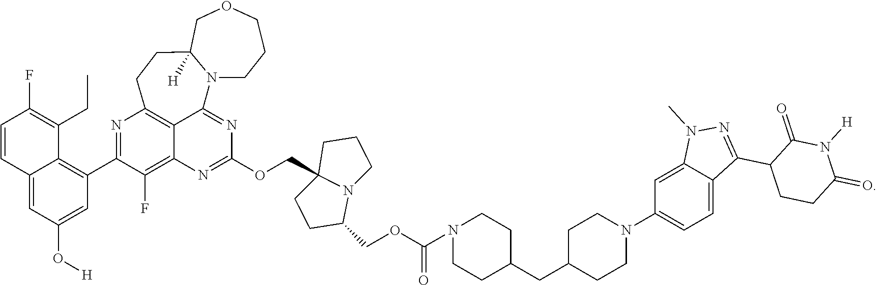

54.-58. (canceled)

59. A pharmaceutical composition comprising a compound of claim 1, or pharmaceutically acceptable salt thereof, and a pharmaceutically acceptable excipient.

60-102. (canceled)
